(19) United States
(12) Patent Application Publication
(10) Pub. No.: US 2025/0320184 A1
(43) Pub. Date: Oct. 16, 2025

(54) SARM1 MODULATORS, PREPARATIONS, AND USES THEREOF

(71) Applicant: SIRONAX LTD., Grand Cayman (KY)

(72) Inventors: Zhaolan ZHANG, Beijing (CN); Lianzhu LIU, Shanghai (CN); Weidong SUN, Beijing (CN); Jianguang HAN, Beijing (CN); Zhiyuan ZHANG, San Diego, CA (US); Xingyu XU, Beijing (CN); Yanping XU, Beijing (CN)

(21) Appl. No.: 18/867,961

(22) PCT Filed: Jun. 6, 2023
(86) PCT No.: PCT/CN2023/098450

§ 371 Date: Nov. 21, 2024

(30) Foreign Application Priority Data
Jun. 7, 2022 (WO) PCT/CN2022/097337
Mar. 23, 2023 (WO) PCT/CN2023/083360

(51) Int. Cl. C07D 221/04; A61K 31/337; A61K 31/366; A61K 31/4015; A61K 31/404; A61K 31/416; A61K 31/4166; A61K 31/421; A61K 31/426; A61K 31/473; A61K 31/517; A61K 31/536; A61K 31/5377; A61K 31/553; C07D 207/38; C07D 209/08; C07D 217/24; C07D 223/16; C07D 231/56; C07D 233/34; C07D 237/32; C07D 239/88; C07D 263/32; C07D 265/18; C07D 267/14; C07D 277/64; C07D 311/20; C07D 401/04; C07D 401/12; C07D 405/12; C07D 413/12; C07D 417/12; C07D 419/14; C07D 471/04; C07D 513/04

(52) U.S. Cl. CPC C07D 221/04; A61K 31/337; A61K 31/366; A61K 31/4015; A61K 31/404; A61K 31/416; A61K 31/4166; A61K 31/421; A61K 31/426; A61K 31/473; A61K 31/517; A61K 31/536; A61K 31/5377; A61K 31/553; C07D 207/38; C07D 209/08; C07D 217/24; C07D 223/16; C07D 231/56; C07D 233/34; C07D 237/32; C07D 239/88; C07D 263/32; C07D 265/18; C07D 267/14; C07D 277/64; C07D 311/20; C07D 401/04; C07D 401/12; C07D 405/12; C07D 413/12; C07D 417/12; C07D 419/14; C07D 471/04; C07D 513/04

(57) ABSTRACT

This disclosure provides compounds of Formula (1), compositions comprising the same, and methods of using the same, including uses in modulating SARM1 and treating various diseases and conditions, e.g., those caused by axonal degeneration.

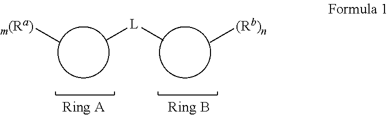

### RELATED APPLICATIONS

This application claims priority to International Application No. PCT/CN2022/097337, filed on Jun. 7, 2022, and International Application No. PCT/CN2023/083360, filed on Mar. 23, 2023. The contents of both applications are incorporated by reference in their entireties.

### FIELD OF THE DISCLOSURE

The present disclosure relates to compounds that modulate SARM1, compositions comprising the compounds, methods of preparing the compounds, and methods of using the compounds to treat various diseases or conditions, e.g., those caused by axonal degeneration.

### BACKGROUND OF THE DISCLOSURE

Axonal degeneration causes disease progression and accumulation of disability in many degenerative diseases of the peripheral nervous system (PNS) and central nervous systems (CNS), such as multiple sclerosis, Parkinson's disease, and amyotrophic lateral sclerosis (ALS), or acute conditions such as traumatic brain injury. (Hughes 2021 (R. Hughes et al., Small Molecule SARM1 Inhibitors Recapitulate the SARM1−/− Phenotype and Allow Recovery of a Metastable Pool of Axons Fated to Degenerate, Cell Rep. 2021 Jan. 5; 34(1):108588.); Bosanac 2021 (T. Bosanac et al., Pharmacological SARM1 inhibition protects axon structure and function in paclitaxel-induced peripheral neuropathy, Brain, Vol. 144, Issue 10, 2021, pages 3226-3238)). Therefore, axonal protection is an important neuroprotective approach to treatment of chronic and acute CNS and PNS neurodegenerative disorders. (Hughes 2021; Bosanac 2021).

SARM1 (Sterile Alpha and TIR Motif-containing 1) is a unique member of the Myd88 family of adaptor proteins and is considered a major driver of an evolutionarily conserved program of axonal degeneration downstream of chemical, inflammatory, mechanical, or metabolic insults to the axon. (Hughes 2021; Bosanac 2021). SARM1 has been recognized as a central mediator of axonal degeneration in a number of diseases or conditions, including ALS, Parkinson's disease, multiple sclerosis, traumatic brain injury, and diabetic neuropathy, as well as chemotherapy induced peripheral neuropathy (CIPN), which is a major cause of morbidity and the main cause of dose reductions and discontinuations in cancer treatment. (Hughes 2021; Bosanac 2021). SARM1 is a compelling target to treat neurodegeneration characterized by axonopathies of the peripheral and central nervous systems

SARM1 contains a mitochondrial targeting sequence, an N-terminal domain with armadillo repeats (ARM), two sterile α-motif (SAM) domains, and a Toll/interleukin-1 receptor (TIR) domain (Gerdts 2013 (J. Gerdts et al., Sarm1-mediated axon degeneration requires both SAM and TIR interactions. J Neurosci. 2013 Aug. 14; 33(33):13569-80.)) The SARM1 TIR domain is a NAD⁺ hydrolase (NADase), which converts the NAD⁺ to ADPR or cADPR and NAM (Sporny 2019 (M. Sporny et al., Structural Evidence for an Octameric Ring Arrangement of SARM1. J Mol Biol. 2019 Sep. 6; 431(19):3591-3605.)). This NADase activity is essential for its axonal degenerative function. (Bosanac 2021). The activity of SARM1 also depends on the oligomerization formed through the SAM domains (Sporny 2019) and is autoinhibited by the ARM domain (Chen (2021) (C. Shen et al., Multiple domain interfaces mediate SARM1 autoinhibition. Proc Natl Acad Sci USA. 2021 Jan. 26; 118(4).)).

Certain SARM1 inhibitors are disclosed by Bosanac 2021, Hughes 2021, Sporny 2020 (M. Sporny et al, Structural basis for SARM1 inhibition and activation under energetic stress. Elife. 2020 Nov. 13; 9:e62021. doi: 10.7554/eLife.62021. PMID: 33185189; PMCID: PMC7688312.), WO 2018/057989 A1, WO 2020/081923 A1, WO 2021/142006 A1, WO2021207302A1, and WO2021207308A1. Certain dipeptidyl peptidase inhibitors (e.g., biphenyl or phenyl benzo imidazole derivatives) are disclosed in US 2005/0272765 A1. Certain benzyl benzoxazol derivatives as Met-kinase inhibitors are disclosed in WO 2008/148449 A1. Certain dihydroisoquinolinone derivatives and combinatorial libraries thereof are described in WO 01/14879. Certain compositions for promoting readthrough of premature termination codons, and methods of using the same are described in WO 2017/049409. Certain nitrogen-containing heterocyclic compounds having nematicidal properties, preparations and uses thereof are described in CN 108276352.

The present disclosure describes SARM1 inhibitors that can be used to prevent axonal degeneration in peripheral and central axonopathies and to provide a transformational disease-modifying treatment for the related diseases or conditions.

### SUMMARY OF THE DISCLOSURE

One aspect of this disclosure provides a compound selected from compounds of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) disclosed herein, a tautomer thereof, a solvate or stereoisomer of the compound or the tautomer, or a pharmaceutically acceptable salt of the foregoing, which can be employed in the treatment of various diseases or conditions, such as diseases or conditions caused by axonal degeneration. For example, disclosed herein is a compound of the following structural Formula 1:

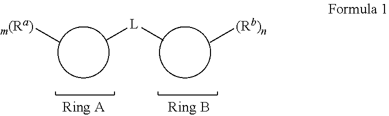

a tautomer thereof, a solvate or stereoisomer of the compound or the tautomer, or a pharmaceutically acceptable salt of the foregoing, wherein:
Ring A is phenyl, a 5- to 10-membered heteroaryl ring, a 5 to 10-membered carbocyclic ring or a 5 to 10-membered heterocyclic ring;
Ring B is phenyl, a 9- to 11-membered aryl ring, a 9- to 11-membered heteroaryl ring, or a 9- to 11-membered heterocyclic ring;
R^{a} is selected from ═O, C₃-C₆ cycloalkyl, —O(C₁-C₄ alkyl), COOH, CN, CONH₂, —C(═O)NH(C₁-C₄ alkyl), —C(═O)N(C₁-C₄ alkyl)₂, —C(═O)O(C₁-C₄ alkyl), halogen, and C₁-C₄ alkyl optionally substituted with 1 to 3 groups selected from halogen, —OH, cyclopropyl, oxetanyl, and azetidinyl;
R^{b} is selected from ═O, OH, halogen, CN, —C(═O)NH₂, —C(═O)NH(C₁-C₄ alkyl), —C(═O)O(C₁-C₄ alkyl), optionally substituted C₃-C₆ cycloalkyl, optionally substituted C₃-C₆ heterocyclyl, optionally substituted C₅-C₆ heteroaryl, —O(C₁-C₄ alkyl), —C(═O)(C₁-C₄ alkyl), —C(═O)(optionally substituted C₃-C₆ cycloalkyl), —C(═O)(optionally substituted 5- to 6-membered heterocyclyl), —C(═O)(optionally substituted 5- to 6-membered heteroaryl), —C(═O)(CH₂)pC(═O)NH₂, —NHC(═O)(C₁-C₄ alkyl), —C(═O)N(C₁-C₄ alkyl)₂, —C(═O)NH(CH₂)pOH, —S(═O)₂NH₂,

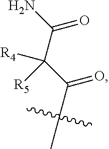

C₁-C₄ alkyl optionally substituted with 1 to 3 groups selected from halogen, NH₂, OH, OCH₃, —C(═O)NH(C₁-C₄ alkyl), —C(═O)O(C₁-C₄ alkyl), —C(═O)N(C₁-C₄ alkyl)₂, —COOH, —C(═O)NH₂, CN, C₃-C₅ cycloalkyl, phenyl, —C(═O)(C₁-C₄ alkyl), —C(═O)(C₃-C₆ cycloalkyl), —C(═O)(5- to 6-membered heterocyclyl), —C(═O)(optionally substituted 5- to 6-membered heteroaryl), C(═O)NH(CH₂)pOH, and 5- to 6-membered heteroaryl,
5- to 6-membered heteroaryl optionally substituted with 1 to 2 groups selected from C₁-C₃ alkyl, ═O, —NH₂, —CN, —CONH₂, halogen, and
4- to 6-membered heterocyclyl optionally substituted with 1 to 2 groups selected from ═O, —OH, —NH₂, —CN, —CONH₂, halogen, and C₁-C₃ alkyl, or
two R^{b} attached to the same position on Ring B join to form a C₃-C₆ cycloalkyl,
wherein R₄ and R₅ are each independently selected from H and C₁ to C₃ alkyl or
R₄ and R₅ join to form a 3- to 5-membered cycloalkyl;
L is selected from

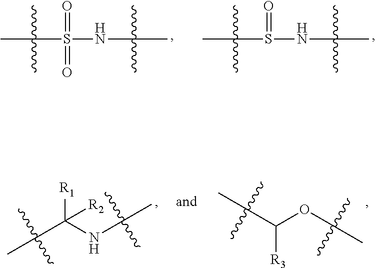

wherein
   R₁ is H, D, or C₁-C₆ alkyl, and R₂ is selected from CN, C₁-C₆ alkenyl, C₃-C₆ cycloalkyl, C₃-C₆ heterocyclyl, 5- or 6-membered heteroaryl, phenyl optionally substituted with 1 to 3 groups selected from C₁-C₄ alkyl and halogen, and
      C₁-C₆ alkyl optionally substituted with 1 to 3 groups selected from halogen, OH, CN, —SO₂CH₃, —NHSO₂CH₃, —CONH₂, OCH₃, and phenyl, or
   R₁ and R₂ join to form a 3- to 6-membered carbocyclyl or 3- to 6-membered heterocyclyl;
   R₃ is C₁-C₄ alkyl optionally substituted with 1 to 3 groups selected from halogen, O(C₁-C₃ alkyl), OH and NH₂;
m is an integer selected from 0, 1, 2, 3, and 4;
n is an integer selected from 0, 1, 2, 3, and 4, and
p is an integer selected from 1, 2, 3, and 4.
provided that the compound is not any of Compound 1A to Compound 121A.

In one aspect of the disclosure, the compounds of the Formulae disclosed herein are selected from Compounds 1 to 391 shown in Table 1, a tautomer thereof, a solvate or stereoisomer of the compound or the tautomer, or a pharmaceutically acceptable salt of the foregoing.

In some embodiments, the disclosure provides pharmaceutical compositions comprising a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 391 and Compounds 1A to 121A shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition is selected amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis a metabolic disease or disorder such as diabetic neuropathy, nonalcoholic fatty liver disease (NAFLD) and nonalcoholic steatohepatitis (NASH) and a peripheral neuropathy like CIPN induced by various drugs.

A further aspect of the disclosure provides methods of treating a disease or condition caused by axonal degeneration, or neuronal damage mediated by SARM1 comprising administering to a subject in need thereof, a therapeutically effective amount of a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administering to a subject in need thereof, a compound selected from Compounds 1 to 391 and Compounds 1A to 121A shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administration of an additional active pharmaceutical agent to the subject in need thereof, either in the same pharmaceutical composition as a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 391 and Compounds 1A to 121A shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., inhibiting, SARM1 in a subject in need thereof, comprising contacting the subject with a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4,44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of modulating, e.g., inhibiting, SARM1 in a subject in need thereof comprise contacting the subject with a compound selected from Compounds 1 to 391 and Compounds 1A to 121A shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

Also disclosed herein are methods of inhibiting or preventing axonal degeneration in a subject in need thereof, comprising contacting the subject with a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of inhibiting or preventing axonal degeneration or neuronal damage mediated by SARM1 in a subject in need thereof comprise contacting the subject with a compound selected from Compounds 1 to 391 and Compounds 1A to 121A shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3-10, 3-8, 3-6, 3-4, or 5-6 carbon atoms. Even further for example, the cycloalkyl group may be a monocyclic group of 3-12, 3-8, 3-6, 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6], and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not an aromatic ring, as “aromatic ring” is defined herein.

The term “heterocyclic” or “heterocycle” or “heterocyclyl” refers to a ring selected from 3- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6], and [6,6] ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds. A double bond may be depicted as [symbol] (two solid lines). The depiction of [symbol] (a solid line and a dashed line), as used herein denotes a bond that may be a double bond or a single bond.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

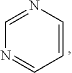

or as a cycle with an inside circle, such as

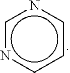

The term “aryl” herein refers to a group selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, I-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R′, —C(═O)NR′R″, or —C(═O)OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, pi-hydroxybutyrate, glycolate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I), or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position.

Combinations of chemical components, e.g., substituents, ring structures, linkers, and/or heteroatoms, envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, ═NR′, ═N—OR′, —NR′R″, —SR′, —SiR′R″R′″, —OC(═O)R′, —C(═O)R′, —CO₂R′, —C(═O)NR′R″, —OC(═O)NR′R″, —NR″C(═O)R′, —NR′—C(═O)NR″R′″, —NR′—SO₂NR″R′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN, —NO₂, —N₃, —CH(Ph)₂, perfluoro(C₁-C₄)alkoxy, and perfluoro(C₁-C₄)alkyl, in a number ranging from zero to three, with those groups having zero, one, or two substituents being particularly preferred. R′, R″, and R′″ each independently refer to hydrogen, unsubstituted C₁-C₈ alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR′R″, —SR′, —SiR′R″R′″, —OC(═O)R′, —C(═O)R′, —CO₂R′, —C(═O)NR′R″, —OC(═O)NR′R″, —NR″C(═O)R′, —NR″CO₂R′, —NR′—SO₂NR″R′″, —S(═O)R′, —SO₂R′, —SO₂NR′R″, —NR″SO₂R, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₆-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, isocyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium, and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., ALS, Parkinson's disease, multiple sclerosis, traumatic brain injury, diabetic neuropathy, and CIPN. The disease or condition may be caused by axonal degeneration. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., ALS, Parkinson's disease, multiple sclerosis, traumatic brain injury, diabetic neuropathy, and CIPN. The disease or condition may be caused by axonal degeneration. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of ±10% with respect to a specific point value) that is recognized by one of ordinary skill in the art.

### II. Compounds and Compositions

In a 1^{st} embodiment, a compound of this disclosure is a compound of the following structural Formula 1:

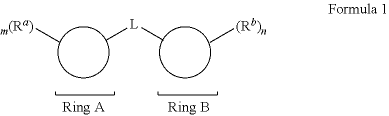

a tautomer thereof, a solvate or stereoisomer of the compound or the tautomer, or a pharmaceutically acceptable salt of the foregoing, wherein:
Ring A is phenyl, a 5- to 10-membered heteroaryl ring, a 5 to 10-membered carbocyclic ring or a 5 to 10-membered heterocyclic ring;
Ring B is phenyl, a 9- to 11-membered aryl ring, a 9- to 11-membered heteroaryl ring, or a 9- to 11-membered heterocyclic ring;
R^{a} is selected from ═O, C₃-C₆ cycloalkyl, —O(C₁-C₄ alkyl), COOH, CN, CONH₂, —C(═O)NH(C₁-C₄ alkyl), —C(═O)N(C₁-C₄ alkyl)₂, —C(═O)O(C₁-C₄ alkyl), halogen, and C₁-C₄ alkyl optionally substituted with 1 to 3 groups selected from halogen, —OH, cyclopropyl, oxetanyl, and azetidinyl;
R^{b} is selected from ═O, OH, halogen, CN, —C(═O)NH₂, —C(═O)NH(C₁-C₄ alkyl), —C(═O)O(C₁-C₄ alkyl), optionally substituted C₃-C₆ cycloalkyl, optionally substituted C₃-C₆ heterocyclyl, optionally substituted C₅-C₆ heteroaryl, —O(C₁-C₄ alkyl), —C(═O)(C₁-C₄ alkyl), —C(═O)(optionally substituted C₃-C₆ cycloalkyl), —C(═O)(optionally substituted 5- to 6-membered heterocyclyl), —C(═O)(optionally substituted 5- to 6-membered heteroaryl), —C(═O)(CH₂)pC(═O)NH₂, —NHC(═O)(C₁-C₄ alkyl), —C(═O)N(C₁-C₄ alkyl)₂, —C(═O)NH(CH₂)pOH, —S(═O)₂NH₂,

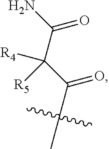

C₁-C₄ alkyl optionally substituted with 1 to 3 groups selected from halogen, NH₂, OH, OCH₃, —C(═O)NH(C₁-C₄ alkyl), —C(═O)O(C₁-C₄ alkyl), —C(═O)N(C₁-C₄ alkyl)₂, —COOH, —C(═O)NH₂, CN, C₃-C₅ cycloalkyl, phenyl, —C(═O)(C₁-C₄ alkyl), —C(═O)(C₃-C₆ cycloalkyl), —C(═O)(5- to 6-membered heterocyclyl), —C(═O)(optionally substituted 5- to 6-membered heteroaryl), C(═O)NH(CH₂)pOH, and 5- to 6-membered heteroaryl,
5- to 6-membered heteroaryl optionally substituted with 1 to 2 groups selected from C₁-C₃ alkyl, ═O, —NH₂, —CN, —CONH₂, halogen, and
4- to 6-membered heterocyclyl optionally substituted with 1 to 2 groups selected from ═O, —OH, —NH₂, —CN, —CONH₂, halogen, and C₁-C₃ alkyl, or
two R^{b} attached to the same position on Ring B join to form a C₃-C₆ cycloalkyl,
wherein R⁴ and R₅ are each independently selected from H and C₁ to C₃ alkyl or R₄ and R₅ join to form a 3 to 5-membered cycloalkyl;
L is selected from

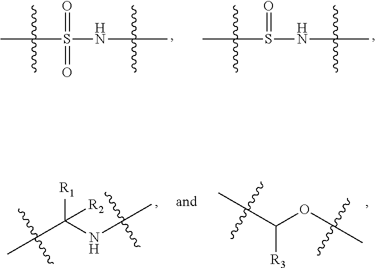

wherein
R₁ is H, D, or C₁-C₆ alkyl, and R₂ is selected from CN, C₁-C₆ alkenyl, C₃-C₆ cycloalkyl, C₃-C₆ heterocyclyl, 5- or 6-membered heteroaryl,
   phenyl optionally substituted with 1 to 3 groups selected from C₁-C₄ alkyl and halogen, and
   C₁-C₆ alkyl optionally substituted with 1 to 3 groups selected from halogen, OH, and phenyl, or
R₁ and R₂ join to form a 3- to 6-membered carbocyclyl or 3- to 6-membered heterocyclyl;
R₃ is C₁-C₄ alkyl optionally substituted with 1 to 3 groups selected from halogen, O(C₁-C₃ alkyl), OH and NH₂;
m is an integer selected from 0, 1, 2, 3, and 4;
n is an integer selected from 0, 1, 2, 3, and 4, and
p is an integer selected from 1, 2, 3, and 4.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In some embodiments, the compound, tautomer, a solvate or stereoisomer of the compound or the tautomer, or pharmaceutically acceptable salt of this disclosure is not any of Compounds 1A to 121A.

In one embodiment, in a compound, tautomer, a solvate or stereoisomer of the compound or the tautomer, or pharmaceutically acceptable salt of this disclosure, Ring A is phenyl, pyridinyl, a 5-membered heteroaryl ring comprising one or two heteroatoms selected for O, S and N, or a 6 to 10-membered heterocyclic ring comprising one or two heteroatoms selected from N and O; and all other variables not specifically defined herein are as defined in the preceding embodiments.

In one embodiment, in a compound, tautomer, a solvate or stereoisomer of the compound or the tautomer, or pharmaceutically acceptable salt of this disclosure, Ring B is phenyl, 9- to 11-membered aryl, 9- to 11-membered heteroaryl ring comprising one or two heteroatoms selected from N, S, and O, or a 9- to 11-membered heterocyclyl ring comprising one or two heteroatoms selected from N, S, and O; and all other variables not specifically defined herein are as defined in any one of the preceding embodiments.

In one embodiment, a compound of the disclosure is a compound of the following structural Formula 2:

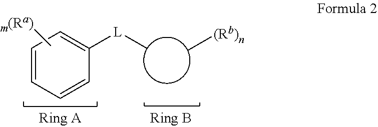

a tautomer thereof, a solvate or stereoisomer of the compound or the tautomer, or a pharmaceutically acceptable salt of the foregoing; and all other variables not specifically defined herein are as defined in any one of the preceding embodiments.

In one embodiment, a compound of the disclosure is a compound of the following structural Formula 2a:

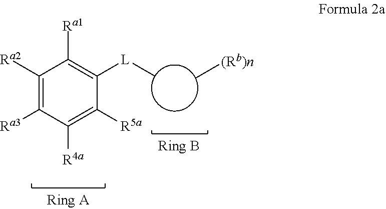

a tautomer thereof, a solvate or stereoisomer of the compound or the tautomer, or a pharmaceutically acceptable salt of the foregoing, wherein zero, one, two, three, or four of R^{a1}, R^{a2}, R^{a3}, R^{a4}, and R^{a5} are independently selected from F, Cl, Br, methyl, CF₃, CF₂H, ethyl, cyclopropyl, and OCH₃, and the rest of R^{a1}, R^{a2}, R^{a3}, R^{a4}, and R^{a5} are H; and all other variables not specifically defined herein are as defined in any one of the preceding embodiments.

In one embodiment, a compound of the disclosure is a compound of the following structural Formula 2b:

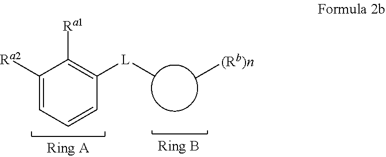

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2c:

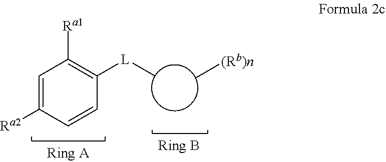

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2d:

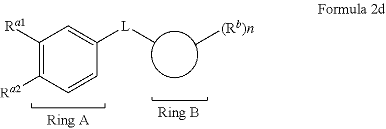

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2e:

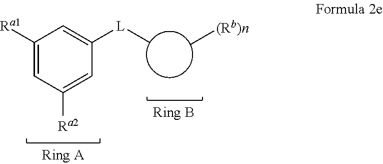

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2f:

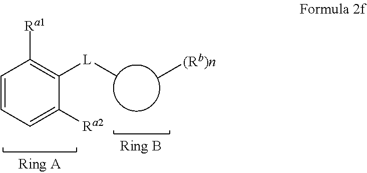

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2g:

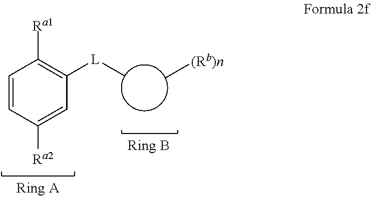

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2h:

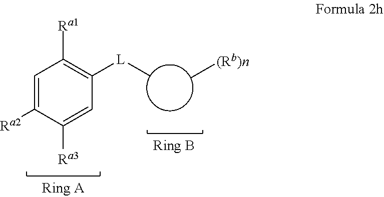

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2i:

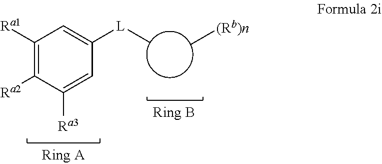

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2j:

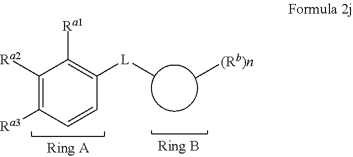

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2k:

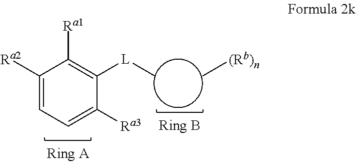

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2L:

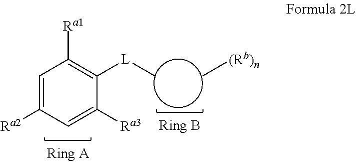

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 2m:

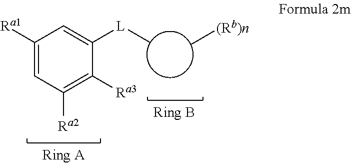

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3:

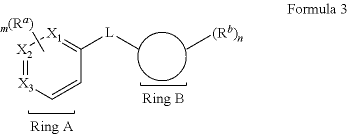

a tautomer thereof, a solvate or stereoisomer of the compound or the tautomer, or a pharmaceutically acceptable salt of the foregoing, wherein one or two of X₁, X₂, and X₃ are N and the rest of X₁, X₂, and X₃ are C; and all other variables not specifically defined herein are as defined in any one of the suitable preceding embodiments.

In one embodiment, a compound of the disclosure is a compound of the following structural Formula 3a:

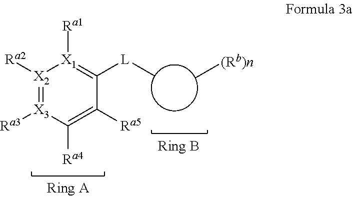

a tautomer thereof, a solvate or stereoisomer of the compound or the tautomer, or a pharmaceutically acceptable salt of the foregoing, wherein one or two of X₁, X₂, and X₃ are N and the rest of X₁, X₂, and X₃ are C, zero, one, two, or three of R^{a1}, R^{a2}, R^{a3}, R^{a4}, and R^{a5} are independently selected from F, Cl, Br, methyl, CF₃, CF₂H, ethyl, cyclopropyl, and OCH₃, provided that the point of attachment on Ring A is not a N, and the rest of R^{a1}, R^{a2}, R^{a3}, R^{a4}, and R^{a5} are H; and all other variables not specifically defined herein are as defined in any one of the suitable preceding embodiments.

In one embodiment, a compound of the disclosure is a compound of the following structural Formula 3b:

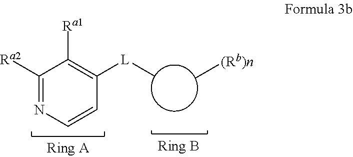

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3c:

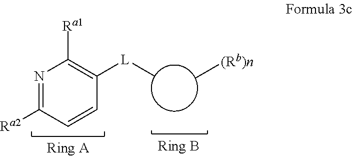

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3d:

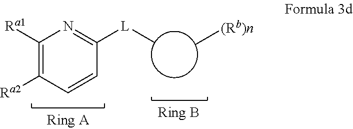

a tautomer thereof, a solvate or stereoisomer of the compound or the tautomer, or a pharmaceutically acceptable salt of the foregoing, wherein R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3e:

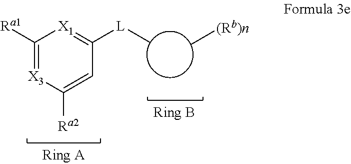

a tautomer thereof, a solvate or stereoisomer of the compound or the tautomer, or a pharmaceutically acceptable salt of the foregoing, wherein X₁ and X₃ are independently selected from C and N, R^{a}I and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3f:

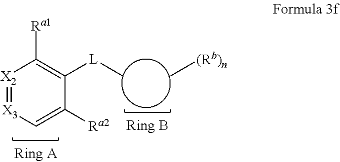

a tautomer thereof, a solvate or stereoisomer of the compound or the tautomer, or a pharmaceutically acceptable salt of the foregoing, wherein X₂ and X₃ are independently selected from C and N, R^{a1} and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3g:

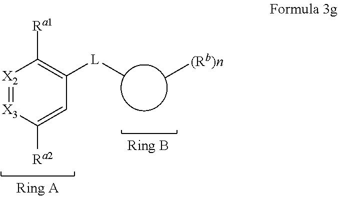

a tautomer thereof, a solvate or stereoisomer of the compound or the tautomer, or a pharmaceutically acceptable salt of the foregoing, wherein X₂ and X₃ are independently selected from C and N, R^{a}I and R^{a2} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3h:

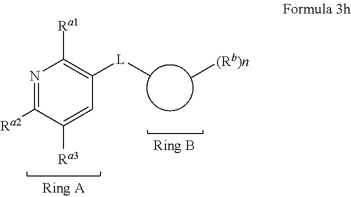

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3i:

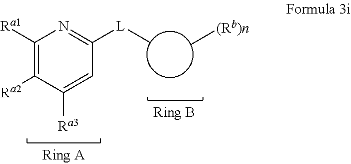

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 3j:

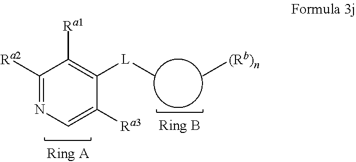

a tautomer thereof, a solvate or stereoisomer of the compound or the tautomer, or a pharmaceutically acceptable salt of the foregoing, wherein one of R^{a1}, R^{a2}, and R^{a3} is selected from F, Cl, Br, methyl, CF₃, CF₂H, ethyl, and OCH₃, the other of them is H; and all other variables not specifically defined herein are as defined in any one of the suitable preceding embodiments.

In one embodiment, a compound of the disclosure is a compound of the following structural Formula 3k:

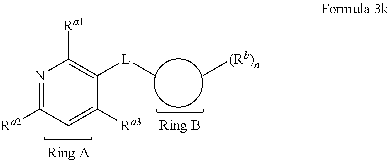

a tautomer thereof, a solvate or stereoisomer of the compound or the tautomer, or a pharmaceutically acceptable salt of the foregoing, wherein one of R^{a1}, R^{a2}, and R^{a3} is selected from F, Cl, Br, methyl, CF₃, CF₂H, ethyl, and OCH₃, the other of them is H; and all other variables not specifically defined herein are as defined in any one of the suitable preceding embodiments.

In one embodiment, a compound of the disclosure is a compound of the following structural Formula 3L:

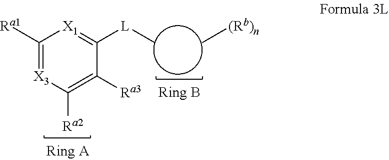

a tautomer thereof, a solvate or stereoisomer of the compound or the tautomer, or a pharmaceutically acceptable salt of the foregoing, wherein X₁ and X₃ are independently selected from C and N, R^{a1}, R^{a2}, and R^{a3} are independently selected from F, Cl, Br, methyl, CF₃, CF₂H, ethyl, and OCH₃; and all other variables not specifically defined herein are as defined in any one of the suitable preceding embodiments.

In one embodiment, a compound of the disclosure is a compound of the following structural Formula 4:

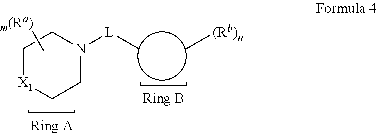

a tautomer thereof, a solvate or stereoisomer of the compound or the tautomer, or a pharmaceutically acceptable salt of the foregoing, wherein X₁ is selected from C, N, and O; and all other variables not specifically defined herein are as defined in any one of the suitable preceding embodiments.

In one embodiment, a compound of the disclosure is a compound of the following structural Formula 5:

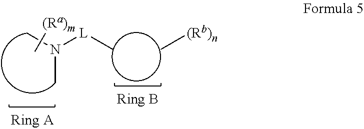

a tautomer thereof, a solvate or stereoisomer of the compound or the tautomer, or a pharmaceutically acceptable salt of the foregoing, wherein Ring A is an 8-membered, 9-membered, or 10-membered heterocyclic ring, wherein the heterocyclic ring is a fused or bridged ring system; and all other variables not specifically defined herein are as defined in any one of the suitable preceding embodiments.

In one embodiment, a compound of the disclosure is a compound of the following structural Formula 6:

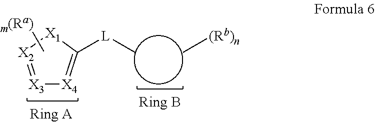

a tautomer thereof, a solvate or stereoisomer of the compound or the tautomer, or a pharmaceutically acceptable salt of the foregoing, wherein one or two of X₁, X₂, X₃, and X₄ are selected from N, O, and S, and the rest of X₁, X₂, X₃, and X₄ are C; and all other variables not specifically defined herein are as defined in any one of the suitable preceding embodiments.

In one embodiment, a compound of the disclosure is a compound of the following structural Formula 7:

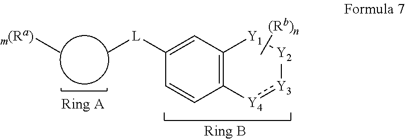

a tautomer thereof, a solvate or stereoisomer of the compound or the tautomer, or a pharmaceutically acceptable salt of the foregoing, wherein zero, one, or two of Y₁, Y₂, Y₃, and Y₄ are N and the rest of Y₁, Y₂, Y₃, and Y₄ are C, R^{b} is selected from ═O, OH, halogen, CN, —C(═O)NH(C₁-C₄ alkyl), —C(═O)O(C₁-C₄ alkyl), and C₁-C₄ alkyl optionally substituted with 1 to 2 groups selected from —C(═O)NH(C₁-C₄ alkyl), —C(═O)O(C₁-C₄ alkyl), —C(═O)N(C₁-C₄ alkyl)₂, —C(═O)NH₂, —COOH, and phenyl, and n is 0, 1, and 2; and all other variables not specifically defined herein are as defined in any one of the suitable preceding embodiments.

In one embodiment, a compound of the disclosure is a compound of the following structural Formula 7a:

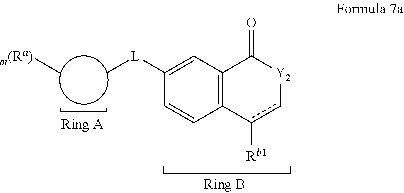

a tautomer thereof, a solvate or stereoisomer of the compound or the tautomer, or a pharmaceutically acceptable salt of the foregoing, wherein Y₂ is C, N, or O, and R^{b1} is selected from H, halogen, OCH₃, CN, C₁ to C₃ alkyl, and C₃ to C₅ cycloalkyl; and all other variables not specifically defined herein are as defined in any one of the suitable preceding embodiments.

In one embodiment, a compound of the disclosure is a compound of the following structural Formula 7b:

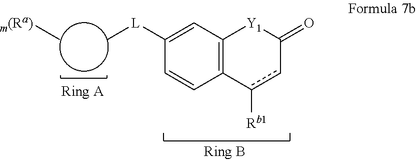

a tautomer thereof, a solvate or stereoisomer of the compound or the tautomer, or a pharmaceutically acceptable salt of the foregoing, wherein Y₁ is C, N, or O, and R^{b1} is selected from H, halogen, and C₁ to C₃ alkyl; and all other variables not specifically defined herein are as defined in any one of the suitable preceding embodiments.

In one embodiment, a compound of the disclosure is a compound of the following structural Formula 8:

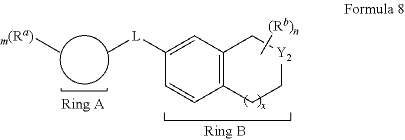

a tautomer thereof, a solvate or stereoisomer of the compound or the tautomer, or a pharmaceutically acceptable salt of the foregoing, wherein Y₂ is selected from N, O, and C, x is 0, 1, and 2, R^{b} is selected from ═O, OH, halogen, CN, —C(═O)NH(C₁-C₄ alkyl), —C(═O)O(C₁-C₄ alkyl), —C(═O)N(C₁-C₄ alkyl)₂, COOH, and C₁-C₄ alkyl optionally substituted with 1 to 2 groups selected from —C(═O)NH(C₁-C₄ alkyl), —C(═O)O(C₁-C₄ alkyl), —C(═O)NH₂, COOH, and phenyl, and n is 0, 1, and 2; and all other variables not specifically defined herein are as defined in any one of the suitable preceding embodiments.

In one embodiment, a compound of the disclosure is a compound of the following structural Formula 8a:

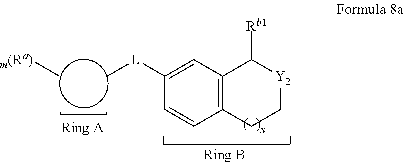

a tautomer thereof, a solvate or stereoisomer of the compound or the tautomer, or a pharmaceutically acceptable salt of the foregoing, wherein Y₂ is selected from N, O, and C, x is 0, 1, and 2, and R^{b1} is selected from H, OH, and ═O; and all other variables not specifically defined herein are as defined in any one of the suitable preceding embodiments.

In one embodiment, a compound of the disclosure is a compound of the following structural Formula 9:

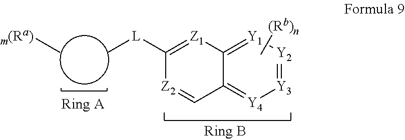

a tautomer thereof, a solvate or stereoisomer of the compound or the tautomer, or a pharmaceutically acceptable salt of the foregoing, wherein zero or one of Y₁, Y₂, Y₃, and Y₄ is N and the rest of Y₁, Y₂, Y₃, and Y₄ are C, zero or one of Z₁ and Z₂ is N and the other is C, R^{b} is selected from ═O, OH, halogen, CN, —C(═O)NH(C₁-C₄ alkyl), —C(═O)O(C₁-C₄ alkyl), —C(═O)N(C₁-C₄ alkyl)₂, OCH₃, and C₁-C₄ alkyl optionally substituted with 1 to 2 groups selected from halogen, —C(═O)NH(C₁-C₄ alkyl), —C(═O)O(C₁-C₄ alkyl), —C(═O)NH₂, and phenyl, and n is 0, 1, and 2; and all other variables not specifically defined herein are as defined in any one of the suitable preceding embodiments.

In one embodiment, a compound of the disclosure is a compound of the following structural Formula 9a:

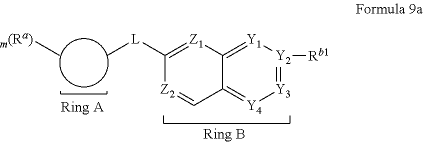

a tautomer thereof, a solvate or stereoisomer of the compound or the tautomer, or a pharmaceutically acceptable salt of the foregoing, wherein zero or one of Y₁, Y₂, Y₃, and Y₄ is N and the rest of Y₁, Y₂, Y₃, and Y₄ are C, zero or one of Z₁ and Z₂ is N and the other is C, R^{b1} is selected from H, halogen, and C₁ to C₃ alkyl, provided that when R^{b1} is not H, Y₂ is C; and all other variables not specifically defined herein are as defined in any one of the suitable preceding embodiments.

In one embodiment, a compound of the disclosure is a compound of the following structural Formula 10:

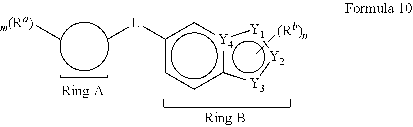

a tautomer thereof, a solvate or stereoisomer of the compound or the tautomer, or a pharmaceutically acceptable salt of the foregoing, wherein zero, one, or two of Y₁, Y₂, Y₃, and Y₄ are N and the rest of Y₁, Y₂, Y₃, and Y₄ are C, R^{b} is selected from ═O, OH, halogen, CN, —C(═O)NH(C₁-C₄ alkyl), —C(═O)O(C₁-C₄ alkyl), —C(═O)N(C₁-C₄ alkyl)₂, COOH, C₃-C₅ cycloalkyl, and C₁-C₄ alkyl optionally substituted with 1 to 2 groups selected from —C(═O)NH(C₁-C₄ alkyl), —C(═O)O(C₁-C₄ alkyl), —C(═O)NH₂, and phenyl, and n is 0, 1, and 2; and all other variables not specifically defined herein are as defined in any one of the suitable preceding embodiments.

In one embodiment, a compound of the disclosure is a compound of the following structural Formula 11:

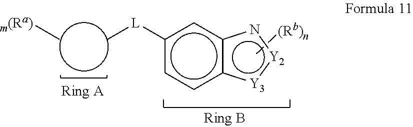

a tautomer thereof, a solvate or stereoisomer of the compound or the tautomer, or a pharmaceutically acceptable salt of the foregoing, wherein one of Y₂ and Y₃ is S or O, and the other is C, R^{b} is selected from ═O, OH, halogen, CN, —C(═O)NH(C₁-C₄ alkyl), —C(═O)O(C₁-C₄ alkyl), and C₁-C₄ alkyl optionally substituted with 1 to 2 groups selected from —C(═O)NH(C₁-C₄ alkyl), —C(═O)O(C₁-C₄ alkyl), —C(═O)NH₂, and phenyl, and n is 0, 1, and 2; and all other variables not specifically defined herein are as defined in any one of the suitable preceding embodiments.

In one embodiment, a compound of the disclosure is a compound of the following structural Formula 12:

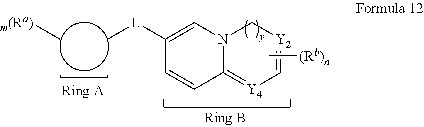

a tautomer thereof, a solvate or stereoisomer of the compound or the tautomer, or a pharmaceutically acceptable salt of the foregoing, wherein one of Y₂ and Y₄ is N and the other is C, y is 0 or 1, R^{b} is selected from ═O, OH, halogen, CN, —C(═O)NH(C₁-C₄ alkyl), —C(═O)O(C₁-C₄ alkyl), and C₁-C₄ alkyl optionally substituted with 1 to 2 groups selected from —C(═O)NH(C₁-C₄ alkyl), —C(═O)O(C₁-C₄ alkyl), —C(═O)NH₂, and phenyl, and n is 0, 1, and 2; and all other variables not specifically defined herein are as defined in any one of the suitable preceding embodiments.

In one embodiment, a compound of the disclosure is a compound of the following structural Formula 13:

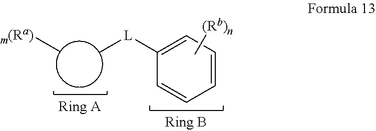

a tautomer thereof, a solvate or stereoisomer of the compound or the tautomer, or a pharmaceutically acceptable salt of the foregoing, wherein R^{b} is selected from:
C₁-C₄ alkyl optionally substituted with 1 to 2 groups selected from —C(═O)NH(C₁-C₄ alkyl), —C(═O)O(C₁-C₄ alkyl), —C(═O)NH₂, and phenyl,
5- to 6-membered heteroaryl optionally substituted with 1 to 2 groups selected from C₁-C₃ alkyl, and
5- to 6-membered heterocyclyl optionally substituted with 1 to 2 groups selected from ═O, C₁-C₃ alkyl, and O(C₁-C₃ alkyl),
   and wherein n is 1, and 2;
   and all other variables not specifically defined herein are as defined in any one of the suitable preceding embodiments.

In one embodiment, a compound of the disclosure is a compound of the following structural Formula 13a:

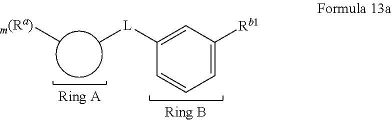

a tautomer thereof, a solvate or stereoisomer of the compound or the tautomer, or a pharmaceutically acceptable salt of the foregoing, wherein R^{b1} is selected from:
5- to 6-membered heteroaryl optionally substituted with 1 to 2 groups selected from C₁-C₃ alkyl and
5- to 6-membered heterocyclyl optionally substituted with 1 to 2 groups selected from ═O and C₁-C₃ alkyl;
   and all other variables not specifically defined herein are as defined in any one of the suitable preceding embodiments.

In one embodiment, a compound of the disclosure is a compound of the following structural Formula 13b:

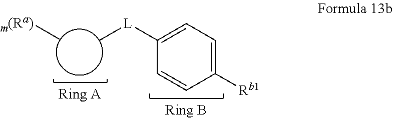

a tautomer thereof, a solvate or stereoisomer of the compound or the tautomer, or a pharmaceutically acceptable salt of the foregoing, wherein R^{b1} is selected from C₁-C₄ alkyl optionally substituted with 1 to 2 groups selected from NH₂, —C(═O)NH(C₁-C₄ alkyl), —C(═O)O(C₁-C₄ alkyl), and —C(═O)NH₂; and all other variables not specifically defined herein are as defined in any one of the suitable preceding embodiments.

In one embodiment, a compound of the disclosure is a compound of the following structural Formula 14:

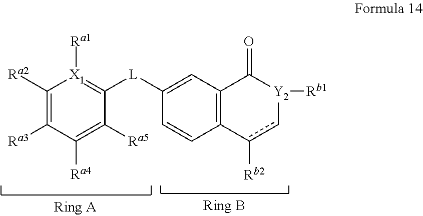

a tautomer thereof, a solvate or stereoisomer of the compound or the tautomer, or a pharmaceutically acceptable salt of the foregoing, wherein X₁ is selected from N and C, Y₂ is selected from N and O, wherein zero, one, two, or three of R^{a1}, R^{a2}, R^{a3}, R^{a4}, and R^{a5} are independently selected from F, Cl, Br, methyl, cyclopropyl, and OCH₃, and the rest of R^{a1}, R^{a2}, R^{a3}, R^{a4}, and R^{a5} are H, provided that when X₁ is N, R^{a1} is absent, R^{b1} is selected from H, CH₂CN, and C₁ to C₂ alkyl optionally substituted with C₃-C₄ cycloalkyl provided that when Y₂ is O, R^{b1} is absent, and R^{b2} is selected from H, halogen, CN, OCH₃, C₁ to C₃ alkyl, and C₃ to C₅ cycloalkyl; and all other variables not specifically defined herein are as defined in any one of the suitable preceding embodiments.

In a 2^{nd} embodiment, a compound of the disclosure is a compound of the following structural Formula 14a:

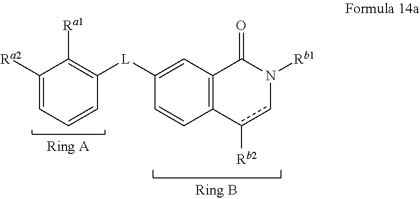

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H, CH₂CN, and C₁ to C₂ alkyl, R^{b2} is selected from H, halogen, CN, OCH₃, C₁ to C₃ alkyl, and C₃ to C₅ cycloalkyl, and L is selected from

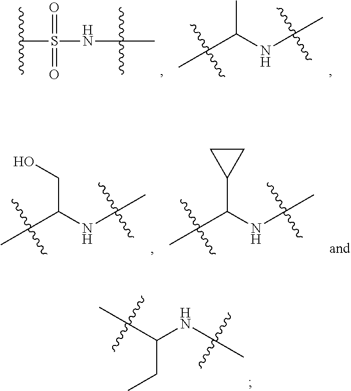

and all other variables not specifically defined herein are as defined in any one of the suitable preceding embodiments.

In a 3^{rd} embodiment, a compound of the disclosure is a compound of the following structural Formula 14b:

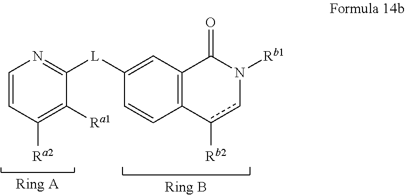

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H and C₁ to C₂ alkyl, R^{b2} is selected from H, halogen, CN, OCH₃, C₁ to C₃ alkyl, and C₃ to C₅ cycloalkyl, and L is selected from

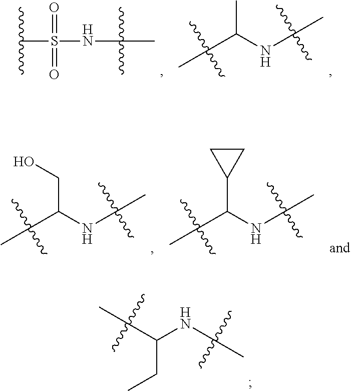

and all other variables not specifically defined herein are as defined in any one of the suitable preceding embodiments.

In a 4^{th} embodiment, a compound of the disclosure is a compound of the following structural Formula 14c:

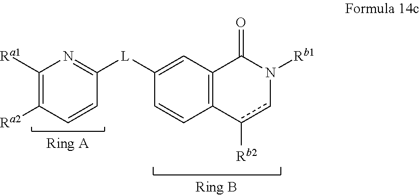

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H and C₁ to C₂ alkyl optionally substituted with C₃-C₄ cycloalkyl, R^{b2} is selected from H, halogen, and C₁ to C₃ alkyl, and L is selected from

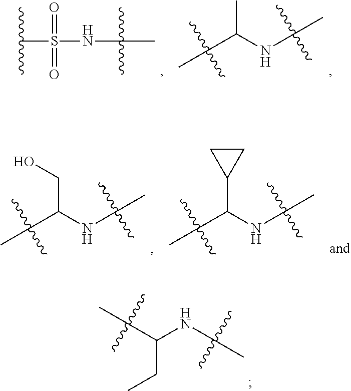

and all other variables not specifically defined herein are as defined in any one of the suitable preceding embodiments.

In a 5^{th} embodiment, a compound of the disclosure is a compound of the following structural Formula 14d:

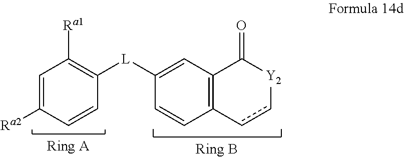

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

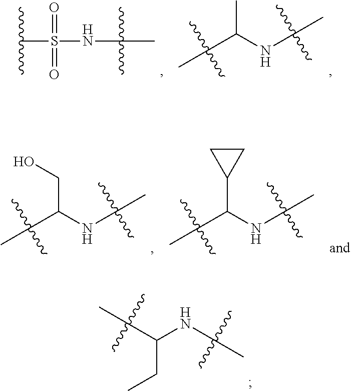

all other variables not specifically defined herein are as defined in any one of the suitable preceding embodiments.

In a 6^{th} embodiment, a compound of the disclosure is a compound of the following structural Formula 14e:

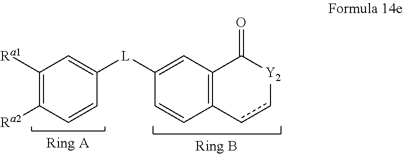

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

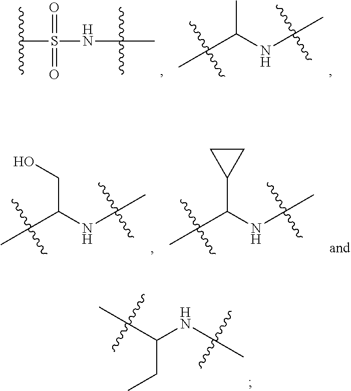

and all other variables not specifically defined herein are as defined in any one of the suitable preceding embodiments.

In a 7^{th} embodiment, a compound of the disclosure is a compound of the following structural Formula 14f:

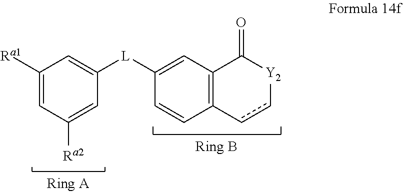

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

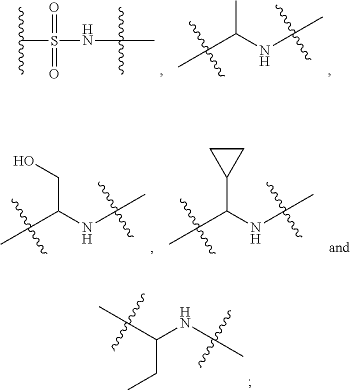

and all other variables not specifically defined herein are as defined in any one of the suitable preceding embodiments.

In a 8^{th} embodiment, a compound of the disclosure is a compound of the following structural Formula 14g:

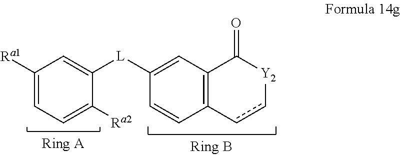

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

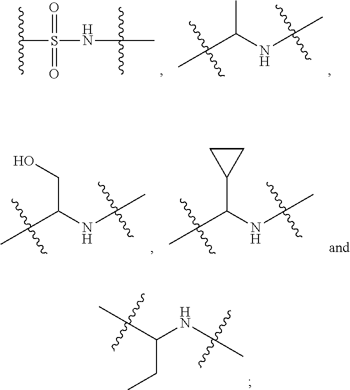

and all other variables not specifically defined herein are as defined in any one of the suitable preceding embodiments.

In a 9^{th} embodiment, a compound of the disclosure is a compound of the following structural Formula 14h-1 or 14h-2:

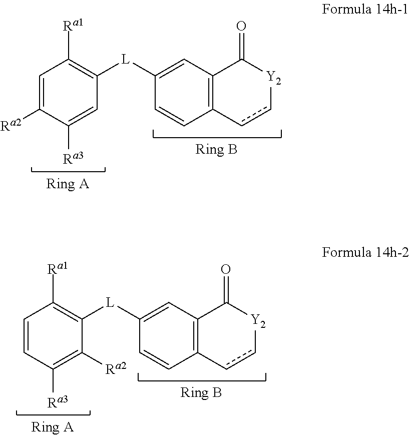

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, and OCH₃, Y₂ is selected from N and O, and L is selected from

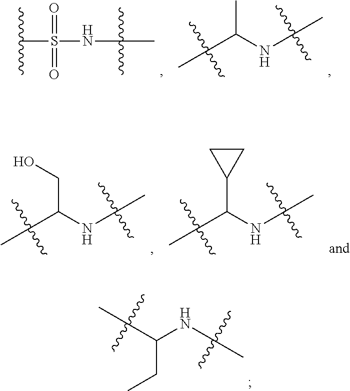

and all other variables not specifically defined herein are as defined in any one of the suitable preceding embodiments.

In a 10^{th} embodiment, a compound of the disclosure is a compound of the following structural Formula 14i:

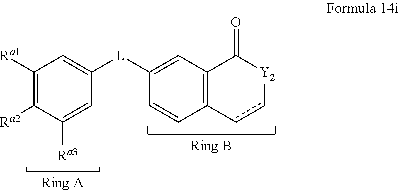

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, and OCH₃, Y₂ is selected from N and O, and L is selected from

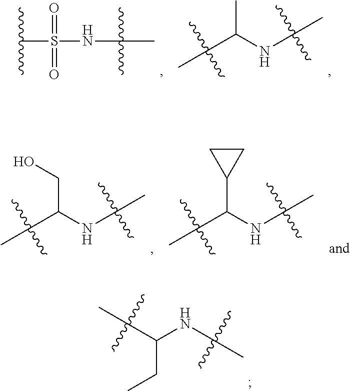

and all other variables not specifically defined herein are as defined in any one of the suitable preceding embodiments.

In a 11^{th} embodiment, a compound of the disclosure is a compound of the following structural Formula 14j:

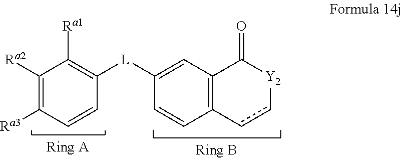

a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, and OCH₃, Y₂ is selected from N and O, and L is selected from

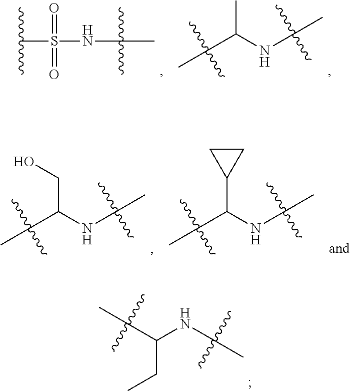

and all other variables not specifically defined herein are as defined in any one of the suitable preceding embodiments.

In one embodiment, a compound of the disclosure is a compound of the following structural Formula 15:

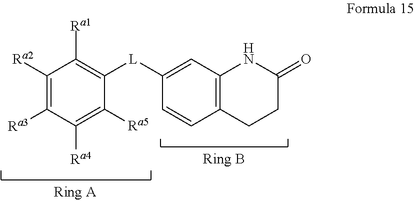

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H; and all other variables not specifically defined herein are as defined in any one of the suitable preceding embodiments.

In a 12^{th} embodiment, a compound of the disclosure is a compound of the following structural Formula 15a:

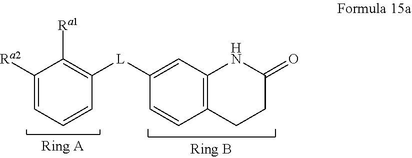

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, and L is selected from

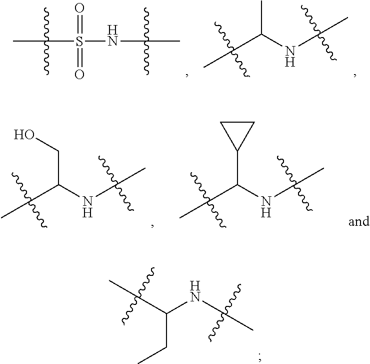

and all other variables not specifically defined herein are as defined in any one of the suitable preceding embodiments.

In a 13^{th} embodiment, a compound of the disclosure is a compound of the following structural Formula 16-1 or 16-2:

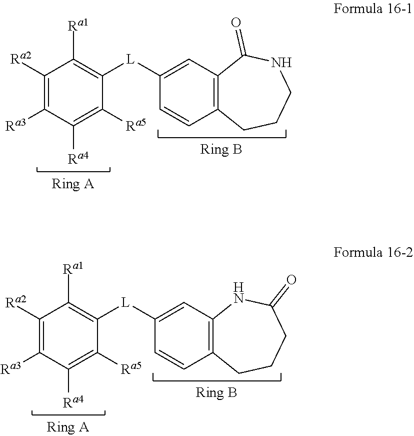

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H; and all other variables not specifically defined herein are as defined in any one of the suitable preceding embodiments.

In a 14^{th} embodiment, a compound of the disclosure is a compound of the following structural Formula 16-1a or 16-2a:

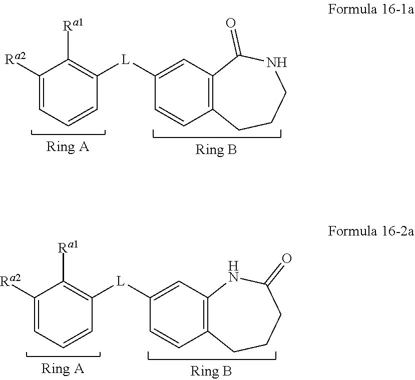

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, and L is selected from

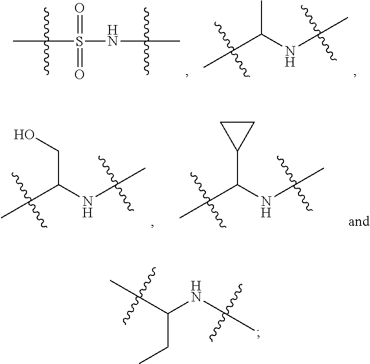

and all other variables not specifically defined herein as defined in any one of the suitable preceding embodiments.

In a 15^{th} embodiment, a compound of the disclosure is a compound of the following structural Formula 17:

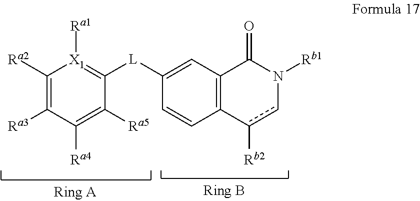

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, R^{b1} is selected from H and C₁ to C₂ alkyl, and R^{b2} is selected from H and C₁ to C₄ alkyl; and all other variables not specifically defined herein are as defined in any one of the suitable preceding embodiments.

In a 16^{th} embodiment, a compound of the disclosure is a compound of the following structural Formula 17a:

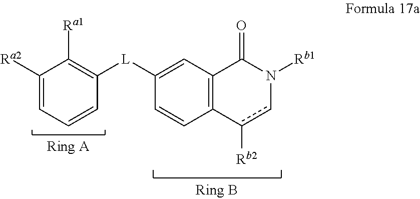

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H and C₁ to C₂ alkyl, R^{b2} is selected from H and C₁ to C₄ alkyl, and L is selected from

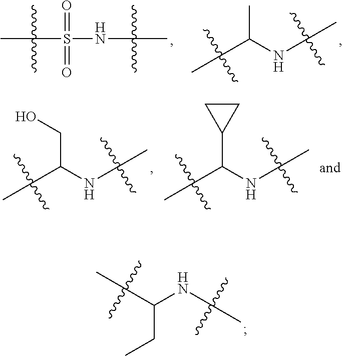

and all other variables not specifically defined herein are as defined in any one of the suitable preceding embodiments.

In a 17^{th} embodiment, a compound of the disclosure is a compound of the following structural Formula 18:

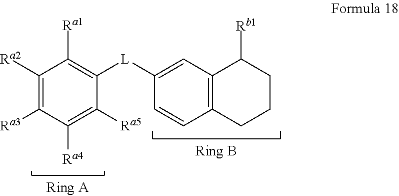

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, R^{b1} is selected from ═O, —NHCOCH₃, and OH; and all other variables not specifically defined herein are as defined in any one of the suitable preceding embodiments.

In a 18^{th} embodiment, a compound of the disclosure is a compound of the following structural Formula 18a:

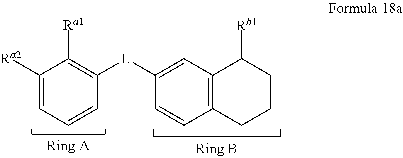

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from ═O, OH, and —NHCOCH₃, and L is selected from

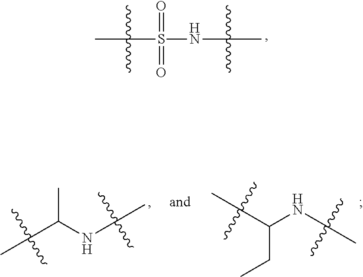

and all other variables not specifically defined herein are as defined in any one of the suitable preceding embodiments.

In one embodiment, a compound of the disclosure is a compound of the following structural Formula 19:

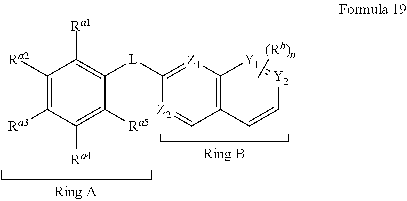

a tautomer thereof, a solvate or stereoisomer of the compound or the tautomer, or a pharmaceutically acceptable salt of the foregoing, wherein none or one of Y₁ and Y₂ is N and the other is C, Z₁, and Z₂ are independently selected from N and C, zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H; and all other variables not specifically defined herein are as defined in any one of the suitable preceding embodiments.

In a 19^{th} embodiment, a compound of the disclosure is a compound of the following structural Formula 20:

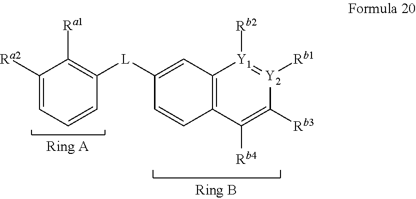

a tautomer thereof, a solvate or stereoisomer of the compound or the tautomer, or a pharmaceutically acceptable salt of the foregoing, wherein none or one of Y₁ and Y₂ is N and the other is C, R^{a1} and R^{a2} are independently selected from F, Cl, Br, methyl, and OCH₃, R^{b1} is selected from H and C₁ to C₂ alkyl provided that when Y₂ is N, R^{b1} is absent, R^{b2} is selected from H, CN, C₁ to C₂ alkyl, and —C(═O)NH₂, provided that when Y₁ is N, R^{b2} is absent, R^{b3} is selected from H and C₁ to C₂ alkyl, R^{b4} is selected from H and C₁ to C₂ alkyl, and L is selected from

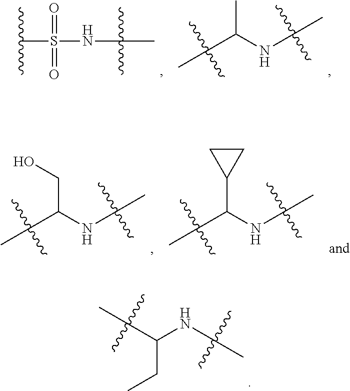

In one embodiment, a compound of the disclosure is a compound of the following structural Formula 21:

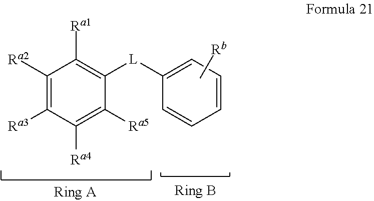

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, R^{b} is selected from —C(═O)NH(C₁-C₄ alkyl), —C(═O)O(C₁-C₄ alkyl), 5- to 6-membered heteroaryl, and 5- to 6-membered heterocyclyl optionally substituted with 1 to 2 groups selected from ═O and C₁-C₃ alkyl; and all other variables not specifically defined herein are as defined in any one of the suitable preceding embodiments.

In a 20^{th} embodiment, a compound of the disclosure is a compound of the following structural Formula 21a:

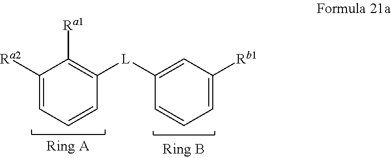

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from 5- to 6-membered heteroaryl, and 5- to 6-membered heterocyclyl optionally substituted with 1 to 2 groups selected from ═O and C₁-C₃ alkyl, and L is selected from

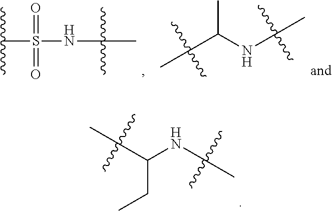

In a 21^{st} embodiment, a compound of the disclosure is a compound of the following structural Formula 21b:

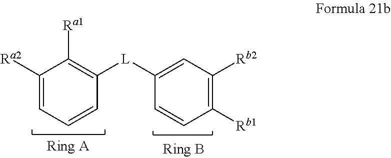

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from —C(═O)NH(C₁-C₄ alkyl), OCH₃, and —C(═O)O(C₁-C₄ alkyl), R^{b2} is selected from H and —C(═O)NH₂, and L is selected from

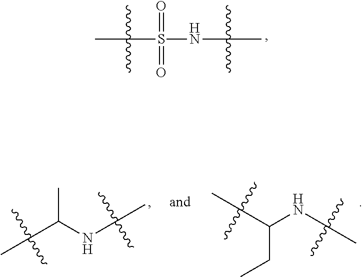

In one embodiment, a compound of the disclosure is a compound of the following structural Formula 22:

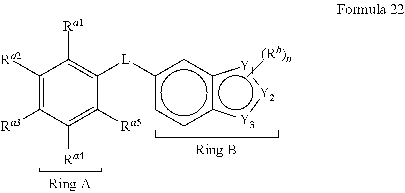

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, zero, one, or two of Y₁, Y₂, and Y₃ are N and the rest of Y₁, Y₂, and Y₃ are C, R^{b} is selected from C₁ to C₂ alkyl, C₃ to C₄ cycloalkyl, and halogen, and n is 0, 1, or 2; and all other variables not specifically defined herein are as defined in any one of the suitable preceding embodiments.

In a 22^{nd} embodiment, a compound of the disclosure is a compound of the following structural Formula 22a:

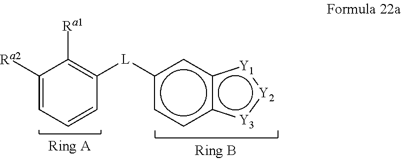

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, zero, one, or two of Y₁, Y₂, and Y₃ are N and the rest of Y₁, Y₂, and Y₃ are C, R^{b} is selected from C₁ to C₂ alkyl, C₃ to C₅ cycloalkyl, CN, C(O)NH₂, and halogen, n is 0, 1, or 2, and L is selected from

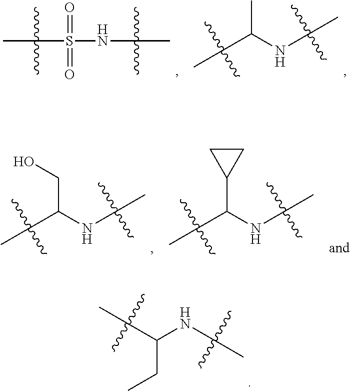

In one embodiment, a compound of the disclosure is a compound of the following structural Formula 23:

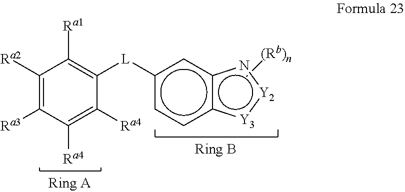

a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, one of Y₂ and Y₃ is S or O, and the other is C; and all other variables not specifically defined herein are as defined in any one of the suitable preceding embodiments.

In a 23^{rd} embodiment, a compound of the disclosure is a compound of the following structural Formula 23a:

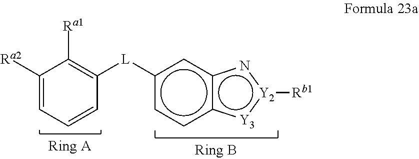

a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H, C₁ to C₃ alkyl, halogen, C₃-C₅ cycloalkyl, CN, and CONH₂, provided that when Y₂ is S or O, R^{b1} is absent, one of Y₂ and Y₃ is S or O, and the other is C, and L is selected from

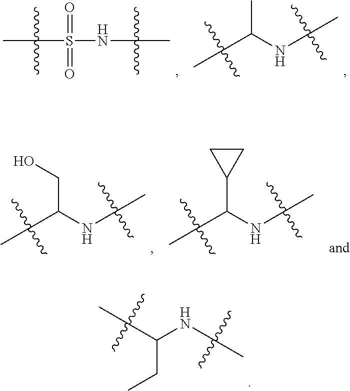

In a 24^{th} embodiment, a compound of the disclosure is a compound of the following structural Formula 24:

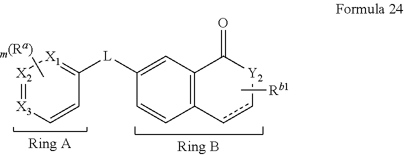

a tautomer thereof, a solvate or stereoisomer of the compound or the tautomer, or a pharmaceutically acceptable salt of the foregoing, wherein one of X₁, X₂, and X₃ is N and the other two of them are C. Y₂ is selected from C, N, and O, and R^{b1} is selected from C₁ to C₂ alkyl, C₃ to C₅ cycloalkyl, O(C₁-C₃ alkyl) and halogen; and all other variables not specifically defined herein are as defined in any one of the suitable preceding embodiments. In some embodiments, L is

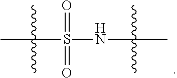 [SMILES: *NS(*)(=O)=O]

In one embodiment, a compound of the disclosure is a compound of the following structural Formula 25:

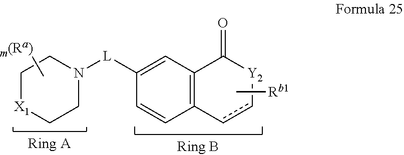

a tautomer thereof, a solvate or stereoisomer of the compound or the tautomer, or a pharmaceutically acceptable salt of the foregoing, wherein X₁ is selected from C, N, and O, Y₂ is selected from C, N and O, and R^{b1} is selected from C₁ to C₂ alkyl, C₃ to C₅ cycloalkyl, O(C₁-C₃ alkyl), and halogen; and all other variables not specifically defined herein are as defined in any one of the suitable preceding embodiments.

In one embodiment, a compound of the disclosure is a compound of the following structural Formula 26:

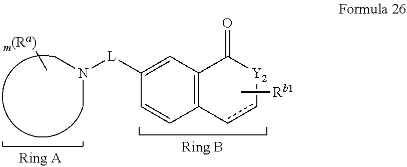

a tautomer thereof, a solvate or stereoisomer of the compound or the tautomer, or a pharmaceutically acceptable salt of the foregoing wherein Ring A is an 8-membered, 9-membered, or 10-membered heterocyclic ring, wherein the heterocyclic ring is a fused or bridged ring system, Y₂ is selected from C, N, and O, and R^{b1} is selected from C₁ to C₂ alkyl, C₃ to C₅ cycloalkyl, O(C₁-C₃ alkyl), and halogen; and all other variables not specifically defined herein are as defined in any one of the suitable preceding embodiments.

In one embodiment, a compound of the disclosure is a compound of the following structural Formula 27:

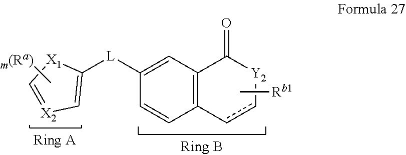

a tautomer thereof, a solvate or stereoisomer of the compound or the tautomer, or a pharmaceutically acceptable salt of the foregoing wherein X₁ is selected from C, O and S, X₂ is selected from N and C, Y₂ is selected from C, N, and O, and R^{b1} is selected from C₁ to C₂ alkyl and halogen; and all other variables not specifically defined herein are as defined in any one of the suitable preceding embodiments.

In a 25^{th} embodiment, a compound of the disclosure is a compound of the following structural Formula 28-1 or 28-2:

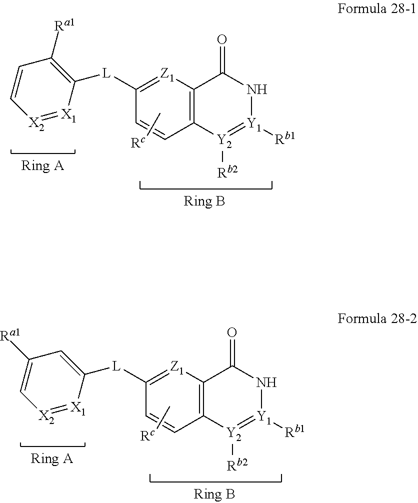

a tautomer thereof, a solvate or stereoisomer of the compound or the tautomer, or a pharmaceutically acceptable salt of the foregoing, wherein X₁, X₂, Y₁, Y₂, and Z₁ are independently selected from C and N, R^{a1} is selected from F, Cl, Br, methyl, CF₃, CF₂H, CN, CONH₂, CH₂NH₂, ethyl, and cyclopropyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{c} is H, halogen, CN, or methyl; and all other variables not specifically defined herein are as defined in any one of the suitable preceding embodiments.

In a 26^{th} embodiment, a compound of the disclosure is a compound of the following structural Formula 29-1 or 29-2:

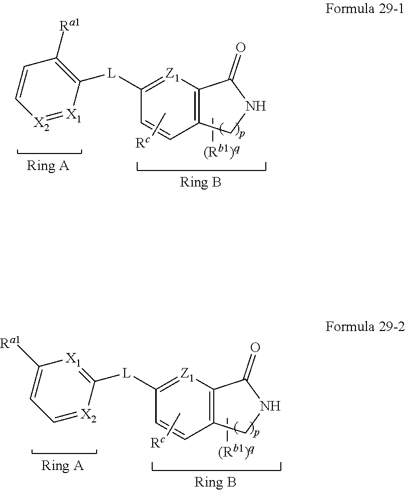

a tautomer thereof, a solvate or stereoisomer of the compound or the tautomer, or a pharmaceutically acceptable salt of the foregoing, wherein X₁, X₂, and Z₁ are independently selected from C and N, R^{a1} is selected from F, Cl, Br, methyl, CF₃, CF₂H, CN, CONH₂, CH₂NH₂, ethyl, and cyclopropyl, R^{b1} is selected from F, Cl, Br, methyl, CN, CF₃, CF₂H, ethyl, CH₂OH, cyclopropyl, and OCH₃, wherein R^{b1} is absent or C₁ to C₄ alkyl when connected to N in Ring B, R^{c} is H, halogen, CN, or methyl, p is 1, 2, or 3, and q is 0, 1, or 2; and all other variables not specifically defined herein are as defined in any one of the suitable preceding embodiments.

In a 27^{th} embodiment, a compound of the disclosure is a compound of the following structural Formula 30-1, 30-2, 30-3, 30-4, or 30-5:

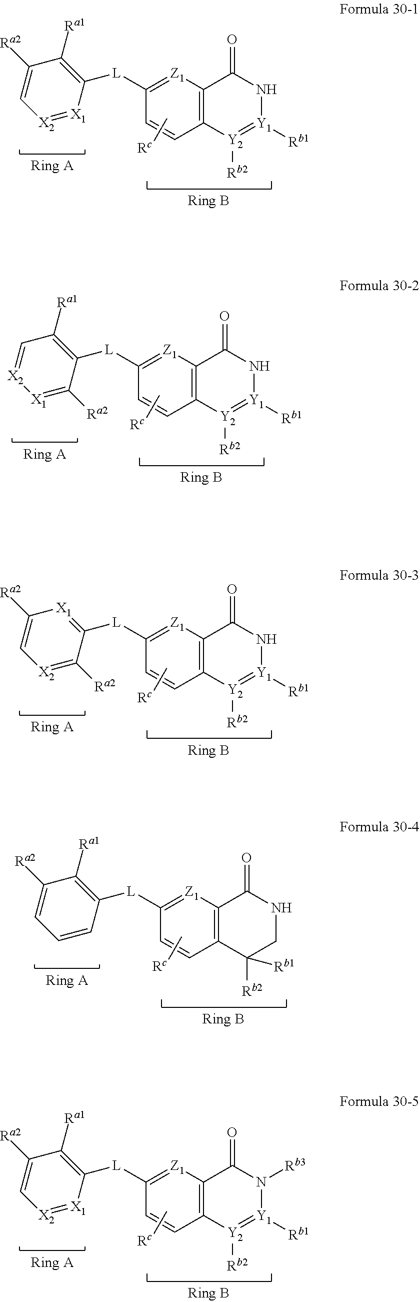

a tautomer thereof, a solvate or stereoisomer of the compound or the tautomer, or a pharmaceutically acceptable salt of the foregoing, wherein X₁, X₂, Y₁, Y₂, and Z₁ are independently selected from C and N, R^{a1} and R^{a2} are each independently selected from F, Cl, Br, methyl, CF₃, CF₂H, ethyl, CN, CONH₂, CH₂NH₂, and cyclopropyl, R^{b}I and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from C₁ to C₄ alkyl optionally substituted with —C(═O)NH₂, 3 to 5-membered cycloalkyl optionally substituted with OH, and 5-6 membered heteroaryl optionally substituted with 1-2 groups selected from F, Cl, Br, Me, CF₃, CF₂H, CN, CONH₂, and NH₂, and R^{c} is H, halogen, CN, or methyl; and all other variables not specifically defined herein are as defined in any one of the suitable preceding embodiments.

In a 28^{th} embodiment, a compound of the disclosure is a compound of the following structural Formula 31-1 or 31-2:

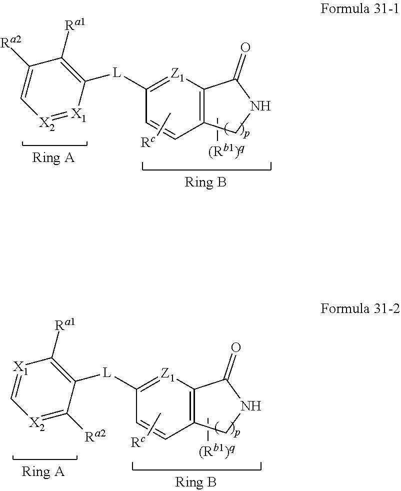

a tautomer thereof, a solvate or stereoisomer of the compound or the tautomer, or a pharmaceutically acceptable salt of the foregoing, wherein X₁, X₂, and Z₁ are independently selected from C and N, R^{a1} and R^{a2} are each independently selected from F, Cl, Br, methyl, CF₃, CF₂H, CN, CONH₂, CH₂NH₂, ethyl, and cyclopropyl, R^{b1} is selected from F, Cl, Br, methyl, CN, CF₃, CF₂H, ethyl, cyclopropyl, and OCH₃, wherein R^{b1} is absent or C₁ to C₄ alkyl when connected to N in Ring B, R^{c} is H, halogen, CN, or methyl, p is 1, 2, or 3, and q is 0, 1, or 2; and all other variables not specifically defined herein are as defined in any one of the suitable preceding embodiments.

In a 29^{th} embodiment, a compound of the disclosure is a compound of the following structural Formula 32-1, 32-2, 32-3, 32-4, or 32-5:

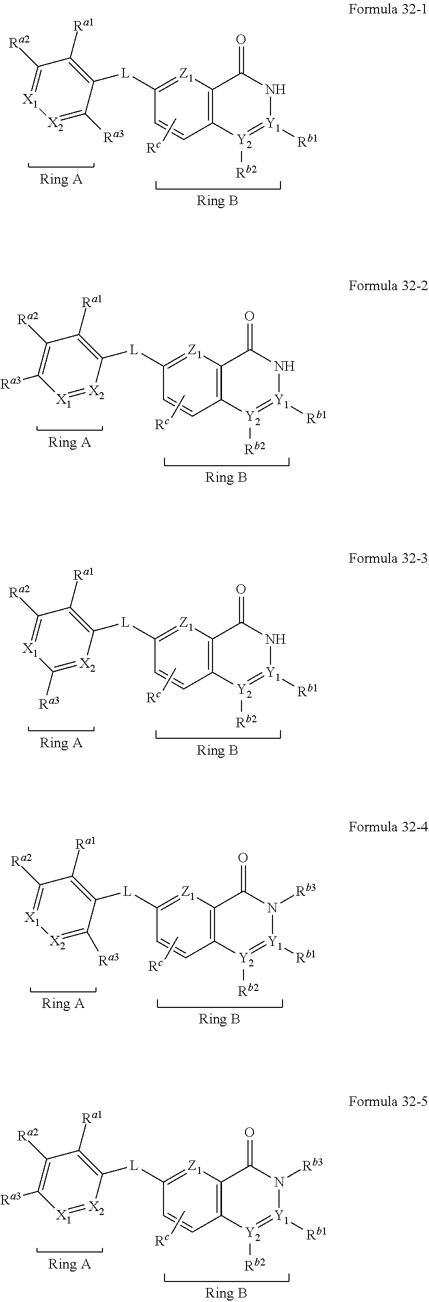

a tautomer thereof, a solvate or stereoisomer of the compound or the tautomer, or a pharmaceutically acceptable salt of the foregoing, wherein X₁, X₂, Y₁, Y₂, and Z₁ are independently selected from C and N, R^{a1}, R^{a2}, and R^{a3} are each independently selected from F, Cl, Br, methyl, CF₃, CF₂H, CN, CONH₂, CH₂NH₂, ethyl, cyclopropyl, and OCH₃, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from H, C₁ to C₄ alkyl optionally substituted with —C(═O)NH₂, 3 to 5-membered cycloalkyl optionally substituted with OH, and 5-6 membered heteroaryl optionally substituted with 1-2 groups selected from F, Cl, Br, Me, CF₃, CF₂H, CN, CONH₂, and NH₂, and R^{c} is H, halogen, CN, or methyl; and all other variables not specifically defined herein are as defined in any one of the suitable preceding embodiments.

In a 30^{th} embodiment, a compound of the disclosure is a compound of the following structural Formula 33-1, 33-2, or 33-3:

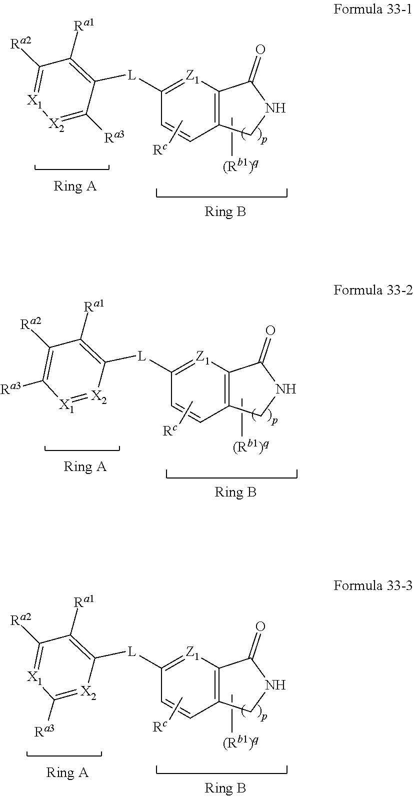

a tautomer thereof, a solvate or stereoisomer of the compound or the tautomer, or a pharmaceutically acceptable salt of the foregoing, wherein X₁, X₂, and Z₁ are independently selected from C and N, R^{a1}, R^{a2}, and R^{a3} are each independently selected from F, Cl, Br, methyl, CF₃, CF₂H, CN, CONH₂, CH₂NH₂, ethyl, cyclopropyl, and OCH₃, R^{b1} is selected from F, Cl, Br, methyl, CN, CF₃, CF₂H, ethyl, cyclopropyl, and OCH₃, wherein R^{b1} is absent or C₁ to C₄ alkyl when connected to N in Ring B, and R^{c} is H, halogen, CN, or methyl, p is 1, 2, or 3, and q is 0, 1, or 2; and all other variables not specifically defined herein are as defined in any one of the suitable preceding embodiments.

In a 31^{st} embodiment, a compound of the disclosure is a compound of the following structural Formula 34-1, 34-2, 34-3, 34-4, 34-5, or 34-6:

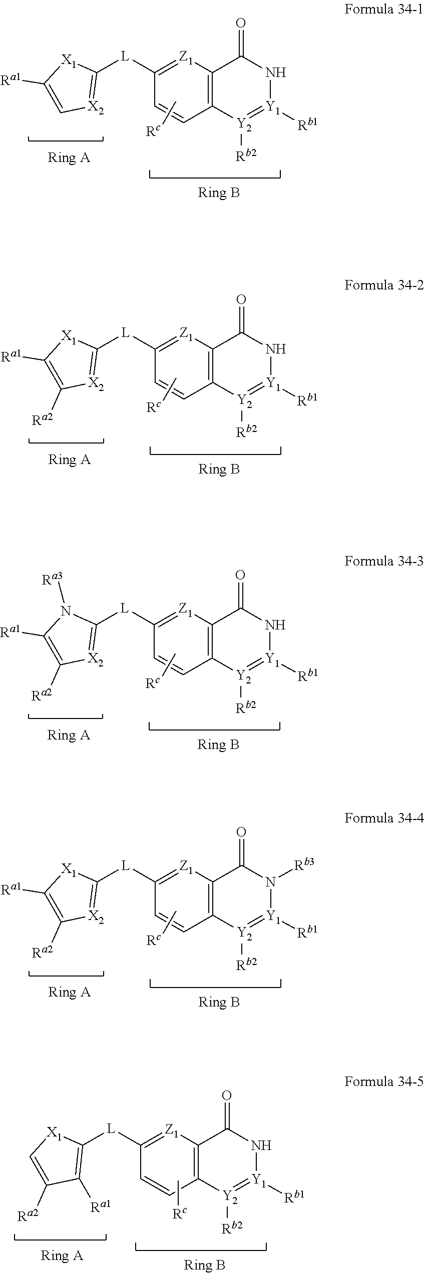

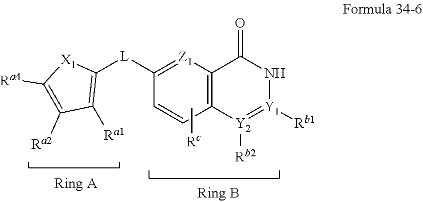

a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2}, and R^{a4} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from H, 5- to 6-membered heteroaryl optionally substituted with 1-2 groups selected from F, Cl, Br, Me, CF₃, CF₂H, CN, CONH₂, and NH₂, and C₁ to C₄ alkyl optionally substituted with —C(═O)NH₂, and R^{c} is H, halogen, CN, or methyl; and all other variables not specifically defined herein are as defined in any one of the suitable preceding embodiments.

In a 32^{nd} embodiment, a compound of the disclosure is a compound of the following structural Formula 35-1, 35-2, 35-3, or 35-4:

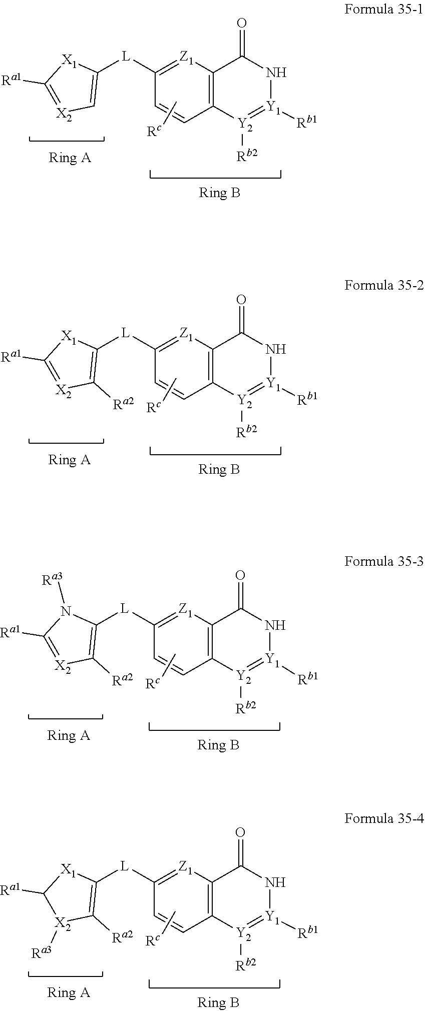

a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R² are each independently selected from F, Cl, Br, C₁ to C₄ alkyl, CF₃, CF₂H, and OCH₃, R^{a3} is C₁ to C₄ alkyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{c} is H, halogen, CN, or methyl; and all other variables not specifically defined herein are as defined in any one of the suitable preceding embodiments.

In a 33^{rd} embodiment, a compound of the disclosure is a compound of the following structural Formula 36-1, 36-2, or and 36-3:

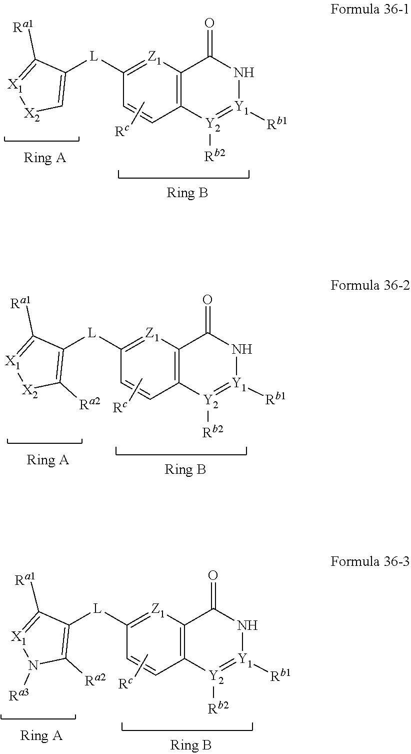

a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{c} is H, halogen, CN, or methyl; and all other variables not specifically defined herein are as defined in any one of the suitable preceding embodiments.

In a 34^{th} embodiment, a compound of the disclosure is a compound of the following structural Formula 37-1, 37-2, 37-3, 37-4, 37-5, or 37-6:

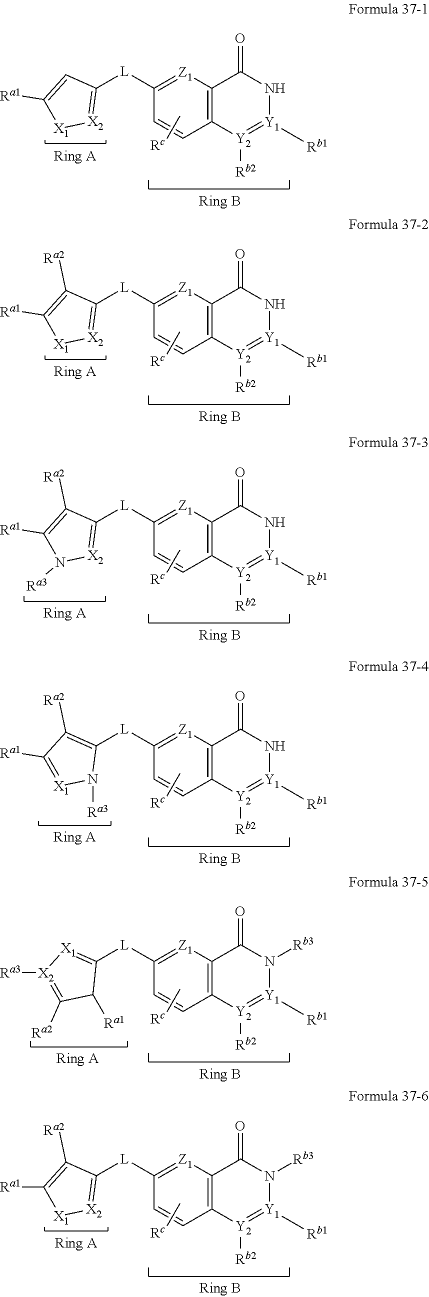

a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2} are each independently selected from F, Cl, Br, C₁ to C₄ alkyl optionally substituted with 1-3 groups of halogen, C₃-C₄ cycloalkyl, CF₃, CF₂H, CN, CONH₂, CH₂NH₂, and OCH₃, R^{a3} is selected from C₃-C₄ cycloalkyl and C₁ to C₄ alkyl optionally substituted by 1 to 3 groups selected from halogen, cyclopropyl, and 4- to 5-membered heterocyclyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from 5- to 6-membered heteroaryl substituted with 1-2 groups selected from F, Cl, Br, Me, CF₃, CF₂H, CN, CONH₂, and NH₂, and C₁ to C₄ alkyl optionally substituted with OH or —C(═O)NH₂, and R^{c} is H, halogen. CN, or methyl; and all other variables not specifically defined herein are as defined in any one of the suitable preceding embodiments.

In a 35^{th} embodiment, a compound of the disclosure is a compound of the following structural Formula 38-1, 38-2, or 38-3:

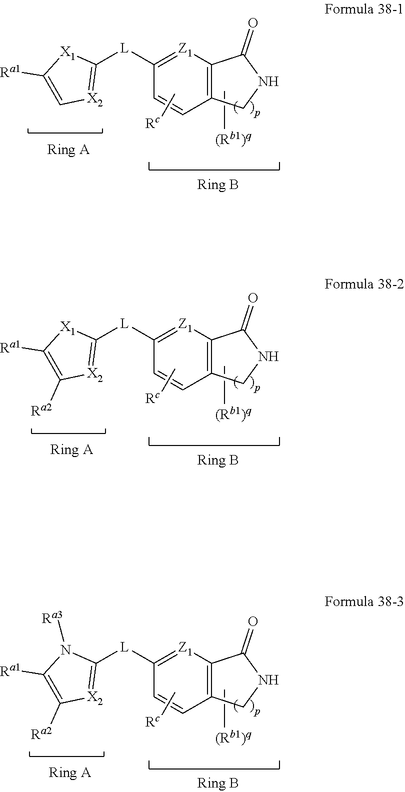

a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, and R^{c} is H, halogen, CN, or methyl, p is 1, 2, or 3, and q is 0, 1, or 2; and all other variables not specifically defined herein are as defined in any one of the suitable preceding embodiments.

In a 36^{th} embodiment, a compound of the disclosure is a compound of the following structural Formula 39-1, 39-2, 39-3, or 39-4:

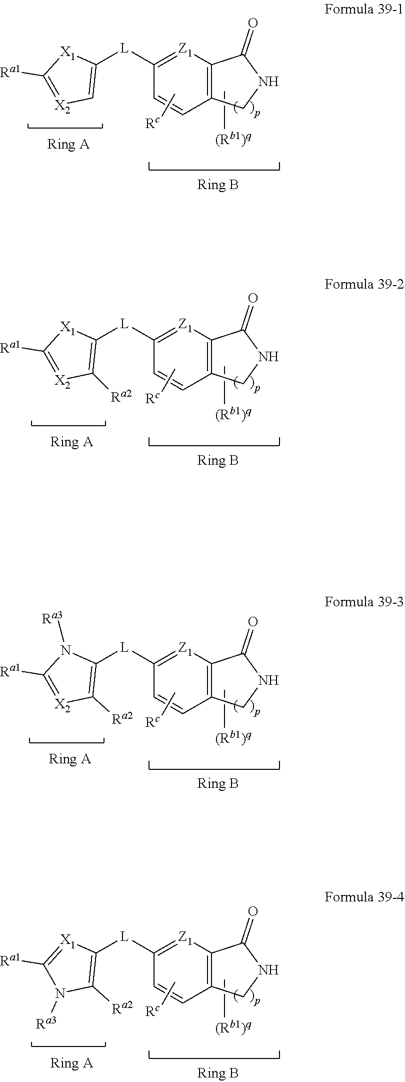

a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, R^{c} is H, halogen, CN, or methyl, p is 1, 2, or 3, and q is 0, 1, or 2; and all other variables not specifically defined herein are as defined in any one of the suitable preceding embodiments.

In a 37^{th} embodiment, a compound of the disclosure is a compound of the following structural Formula 40-1, 40-2, or 40-3:

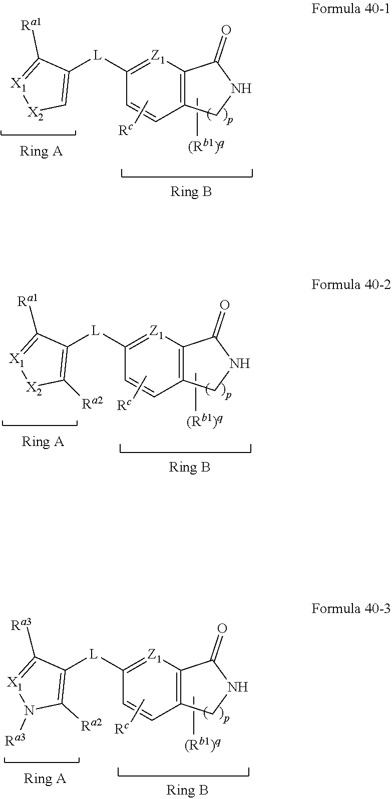

a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, R^{c} is H, halogen, CN or methyl, p is 1, 2, or 3, and q is 0, 1, or 2; and all other variables not specifically defined herein are as defined in any one of the suitable preceding embodiments.

In a 38^{th} embodiment, a compound of the disclosure is a compound of the following structural Formula 41-1, 41-2, 41-3, 41-4, or 41-5:

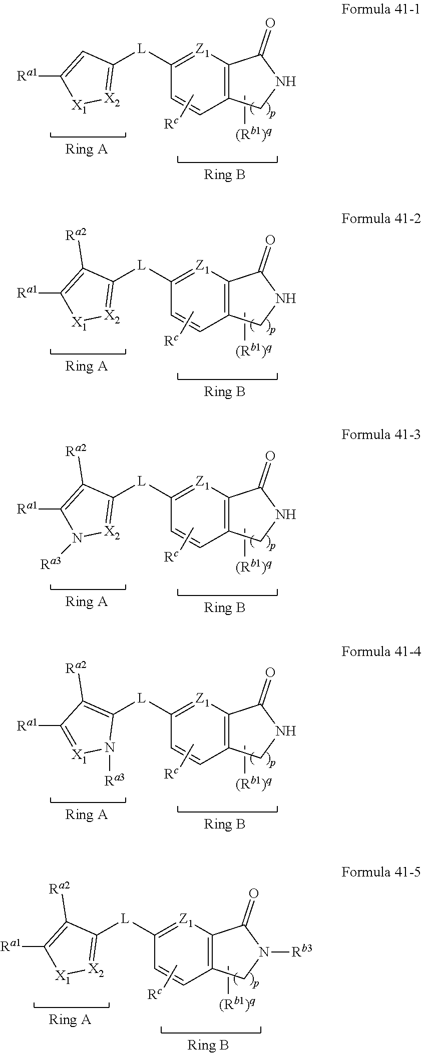

a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, R^{b3} is selected from C₁ to C₄ alkyl optionally substituted with OH or —C(═O)NH₂, R^{c} is H, halogen, CN, or methyl, p is 1, 2, or 3, and q is 0, 1, or 2; and all other variables not specifically defined herein are as defined in any one of the suitable preceding embodiments.

In a 39^{th} embodiment, a compound of the disclosure is a compound of the following structural Formula 42-1, 42-2, or 42-3:

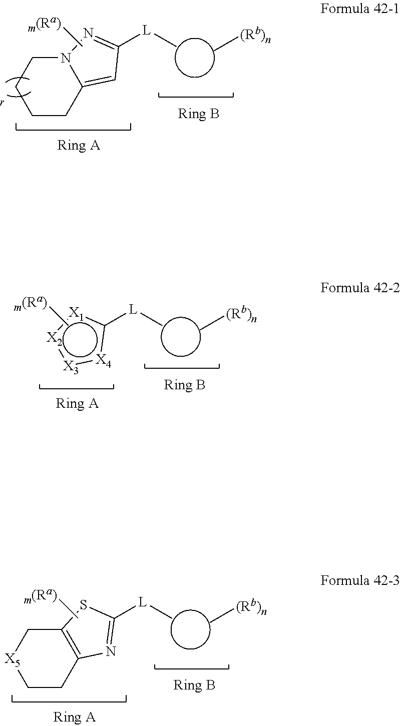

a tautomer thereof, a solvate or stereoisomer of the compound or the tautomer, or a pharmaceutically acceptable salt of the foregoing, wherein X₁, X₂, X₃, and X₄ are independently selected from C, N, and S, and wherein X₅ is selected from C and N, and r is an integer selected from 0, 1, and 2; and all other variables not specifically defined herein are as defined in any one of the suitable preceding embodiments.

In a 40^{th} embodiment, a compound of the disclosure is a compound of the following structural Formula 43-1, 43-2, 43-3, or 43-4:

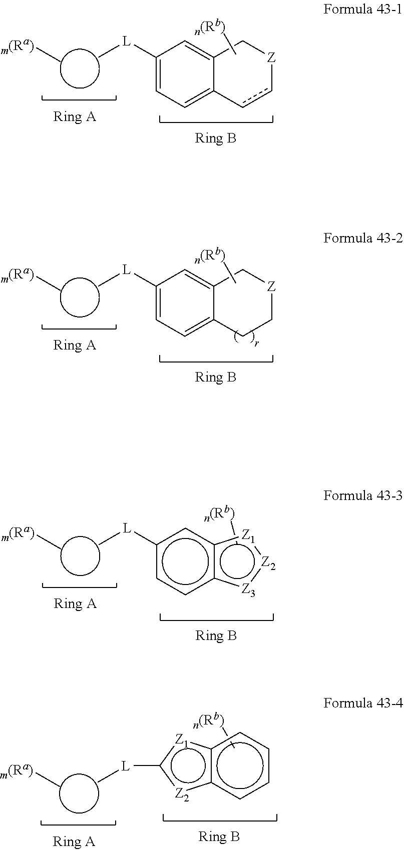

a tautomer thereof, a solvate or stereoisomer of the compound or the tautomer, or a pharmaceutically acceptable salt of the foregoing, wherein Z, Z₁, Z₂, and Z₃ are independently selected from C, S, and N, and r is an integer selected from 0, 1, and 2; and all other variables not specifically defined herein are as defined in any one of the suitable preceding embodiments.

In a 41^{st} embodiment, a compound of the disclosure is a compound of the following structural Formula 44-1, 44-2, 44-3, 44-4, 44-5, 44-6, 44-7, 44-8, 44-9, 44-10, 44-11, 44-12, 44-13, or 44-14:

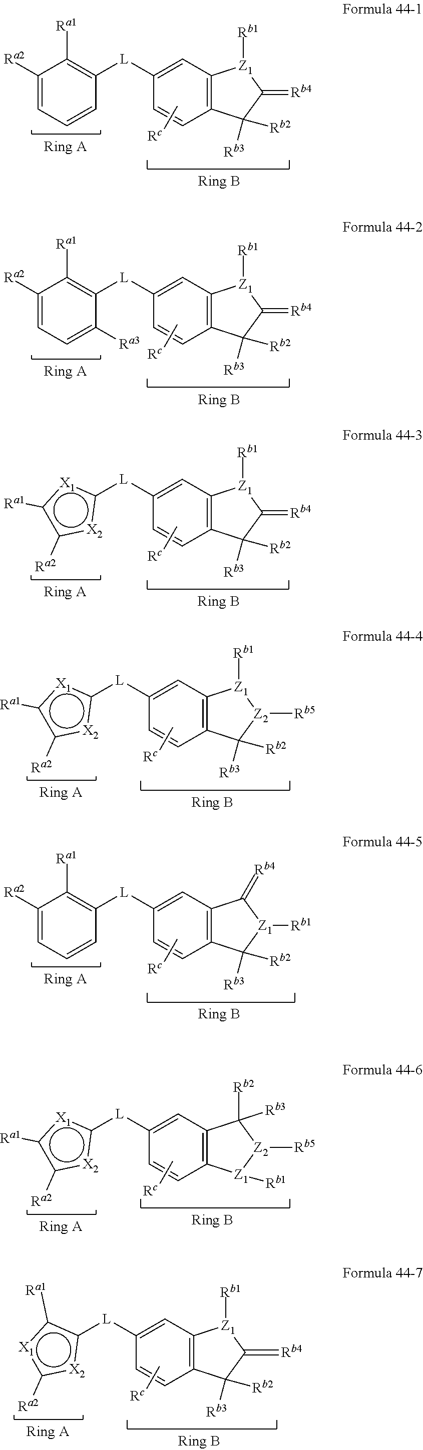

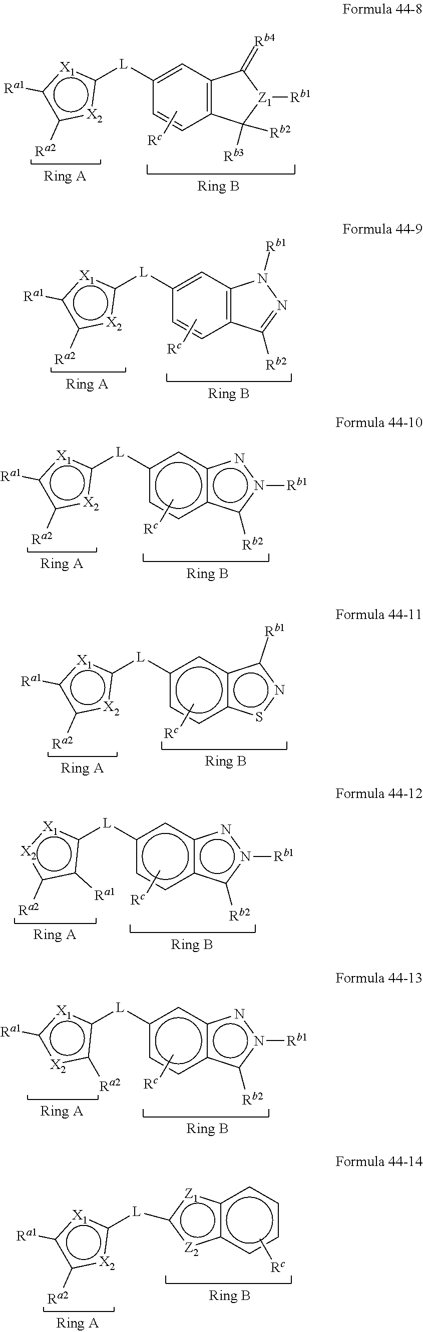

a tautomer thereof, a solvate or stereoisomer of the compound or the tautomer, or a pharmaceutically acceptable salt of the foregoing, wherein:
X₁ is selected from C, S, O and N, X₂ is selected from C, N, S, and O, Z₁ and Z₂ are independently selected from C, N, and S,
R^{a1}, R^{a2}, and R^{a3} are each independently selected from F, Cl, Br, C₁ to C₄ alkyl, CF₃, CF₂H, CN, CONH₂, CH₂NH₂, and OCH₃,
R^{b1} is selected from H, F, Cl, Br, methyl, CN, CF₃, CF₂H, ethyl, OCH₃, —C(═O)CH₂C(═O)NH₂, —C(═O)(C₁ to C₄ alkyl), —C(═O)(C₃ to C₅ cycloalkyl), —C(═O)(5- to 6-membered heteroaryl), —C(═O)(4- to 6-membered heterocyclyl optionally substituted with ═O or OH), —C(═O)OH, OH,

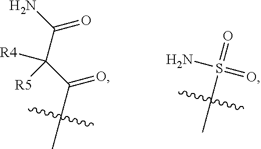

—C(═O)NH₂, 3- to 5-membered cycloalkyl optionally substituted by OH, and C₁ to C₄ alkyl optionally substituted by OH or —C(═O)NH₂,
R^{b2} and R^{b3} are independently selected from H, F, Cl, Br, methyl, CN, CF₃, CF₂H, ethyl, —C(═O)NH₂, and OCH₃, or R^{b2} and R^{b3} join to form a C₃ to C₅ cycloalkyl,
R^{b4} is ═O or absent,
R^{b5} is selected from H, F, Cl, Br, methyl, CN, CF₃, CF₂H, ethyl, OCH₃, —C(═O)CH₂C(═O)NH₂, —C(═O)(C₁ to C₄ alkyl), —C(═O)(C₃ to C₅ cycloalkyl), —C(═O)(5- to 6-membered heteroaryl), —C(═O)(5- to 6-membered heterocyclyl optionally substituted with ═O), —C(═O)OH, OH, C₁ to C₄ alkyl optionally substituted by OH or —C(═O)NH₂, and C₃ to C₅ cycloalkyl optionally substituted with OH, and
R^{c} is H, halogen, CN, or methyl, and
wherein R₄ and R₅ join to form a 3 to 4-membered cycloalkyl; and all other variables not specifically defined herein are as defined in any one of the suitable preceding embodiments.

In a 42^{nd} embodiment, a compound of the disclosure is a compound of the following structural Formula 45-1 or 45-2:

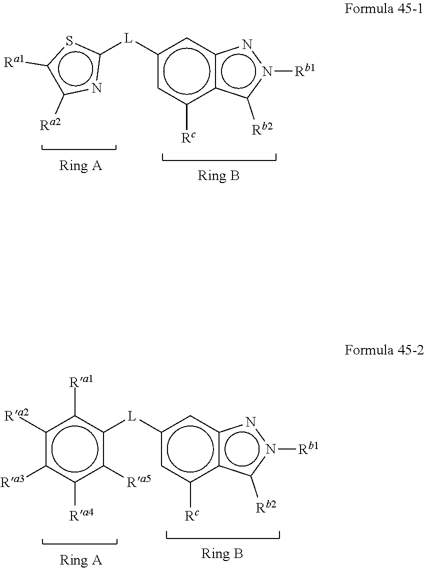

a tautomer thereof, a solvate or stereoisomer of the compound or the tautomer, or a pharmaceutically acceptable salt of the foregoing, wherein:
R^{a1} and R^{a2} are each independently selected from halogen and C₁ to C₂ alkyl optionally substituted with 1 to 3 groups of halogen,
R′^{a1}, R′^{a2}, R′^{a3}, R′^{a4}, and R′^{a5} are each independently selected from H and halogen, wherein 2, 3, or 4 of R′^{a1}, R′^{a2}, R′^{a3}, R′^{a4}, and R′^{a5} are halogen and the rest is H,
R^{b1} is selected from H and C₁ to C₄ alkyl optionally substituted by —C(═O)NH₂,
R^{b2} is selected from H and C₁ to C₄ alkyl optionally substituted by 1-3 groups of halogen, and
R^{c} is selected from H, halogen, and C₁ to C₂ alkyl; and all other variables not specifically defined herein are as defined in any one of the suitable preceding embodiments.

In a 43^{rd} embodiment, a compound of the disclosure is a compound of the following structural Formula 46-1, 46-2, or 46-3:

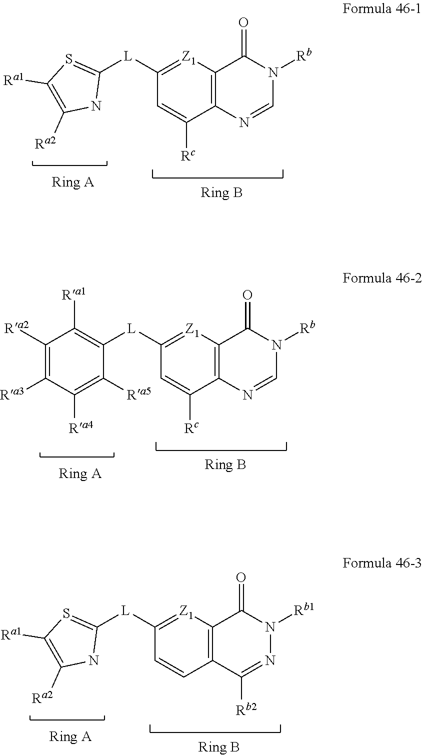

a tautomer thereof, a solvate or stereoisomer of the compound or the tautomer, or a pharmaceutically acceptable salt of the foregoing, wherein:
Z₁ is selected from C and N,
R^{a1} and R^{a2} are each independently selected from halogen and C₁ to C₂ alkyl optionally substituted with 1 to 3 groups of halogen,
R′^{a1}, R′^{a2}, R′^{a3}, R′^{a4}, and R′^{a5} are each independently selected from H and halogen, wherein 2, 3, or 4 of R′^{a1}, R′^{a2}, R′^{a3}, R′^{a4}, and R′^{a5} are halogen and the rest is H,
R^{b} is selected from H and 5- to 6-membered heteroaryl containing 2 to 3 heteroatoms selected from N and S, wherein the 5- to 6-membered heteroaryl of R^{b} is optionally substituted by 1 to 3 groups selected from halogen and C₁ to C₄ alkyl,
R^{b1} is selected from H and C₁ to C₄ alkyl optionally substituted by —C(═O)NH₂,
R^{b2} is selected from H, halogen, —O(C₁ to C₄ alkyl), and C₁ to C₄ alkyl optionally substituted with 1-3 groups of halogen, and
R^{c} is selected from H, halogen, and C₁ to C₂ alkyl; and all other variables not specifically defined herein are as defined in any one of the suitable preceding embodiments.

In a 44^{th} embodiment, in a compound, tautomer, a solvate or stereoisomer of the compound or the tautomer, or pharmaceutically acceptable salt of this disclosure, Ring A substituted with m groups of R^{a} is selected from:

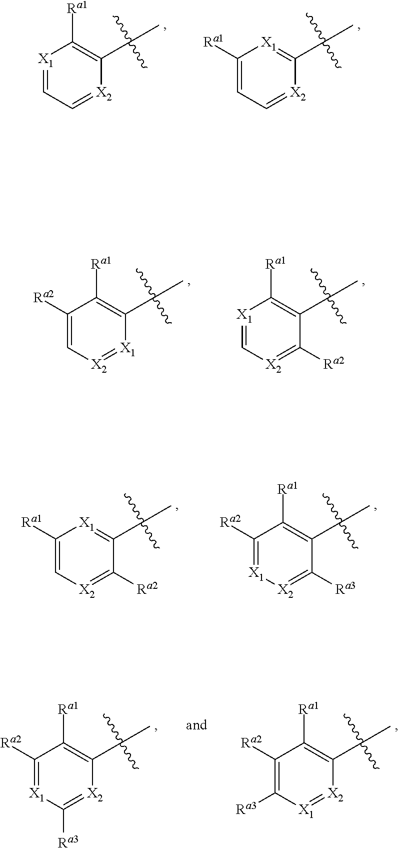

wherein R^{a1}, R^{a2}, and R^{a3} are independently selected from F, Cl, Br, methyl, CF₃, CF₂H, and cyclopropyl, and X₁ and X₂ are independently selected from C and N; and all other variables not specifically defined herein are as defined in any one of the suitable preceding embodiments.

In a 45^{th} embodiment, in a compound, tautomer, a solvate or stereoisomer of the compound or the tautomer, or pharmaceutically acceptable salt of this disclosure, Ring A substituted with m groups of R^{a} is selected from:

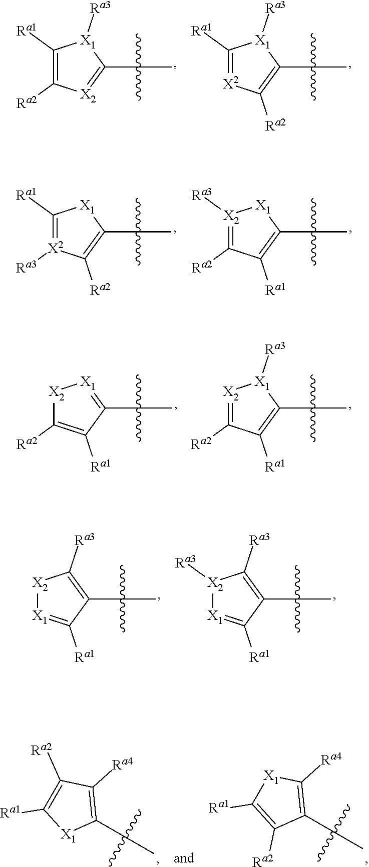

wherein R^{a1} and R^{a2} are independently selected from H, CN, F, Cl, Br, C₁ to C₄ alkyl, CF₃, CF₂H, and OCH₃, R^{a3} is H, or C₁ to C₄ alkyl, R^{a4} is selected from H, F, Cl, Br, and C₁ to C₄ alkyl, and X₁ and X₂ are independently selected from N, O, and S; and all other variables not specifically defined herein are as defined in any one of the suitable preceding embodiments.

In a 46^{th} embodiment, in a compound, tautomer, a solvate or stereoisomer of the compound or the tautomer, or pharmaceutically acceptable salt of this disclosure, Ring A is selected from:

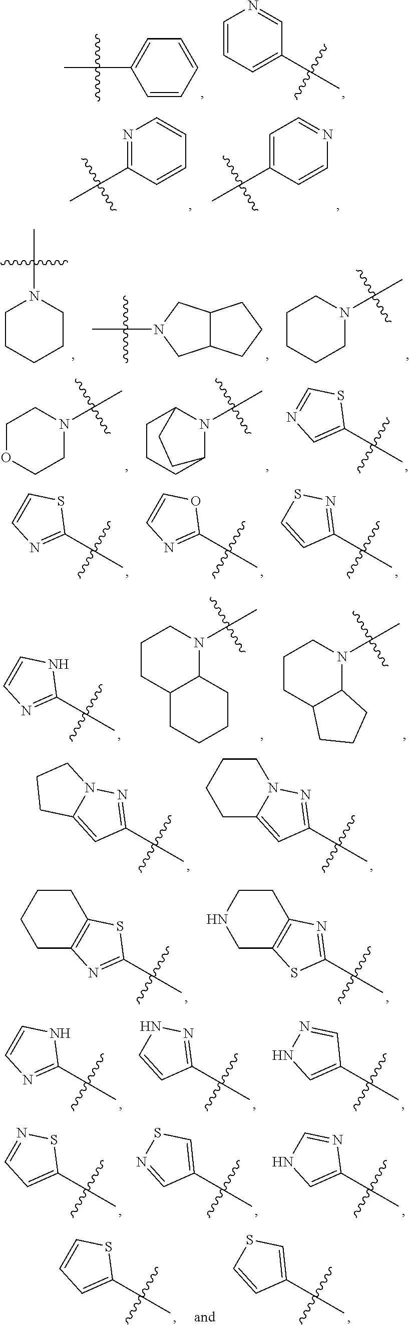

wherein Ring A is substituted with m groups of R^{a}; and all other variables not specifically defined herein are as defined in any one of the suitable preceding embodiments.

In a 47^{th} embodiment, in a compound, tautomer, a solvate or stereoisomer of the compound or the tautomer, or pharmaceutically acceptable salt of this disclosure, Ring A substituted with m groups of R^{a} is selected from:

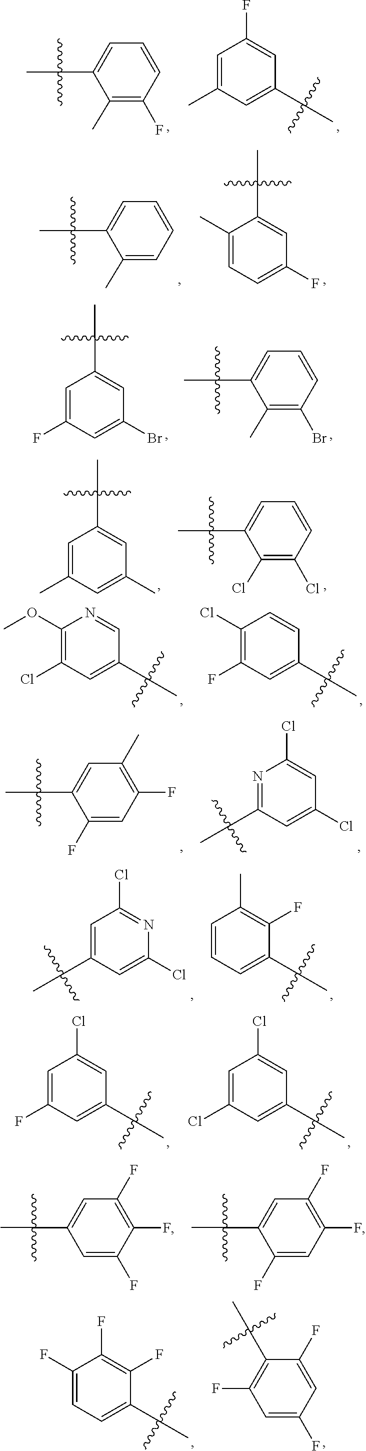

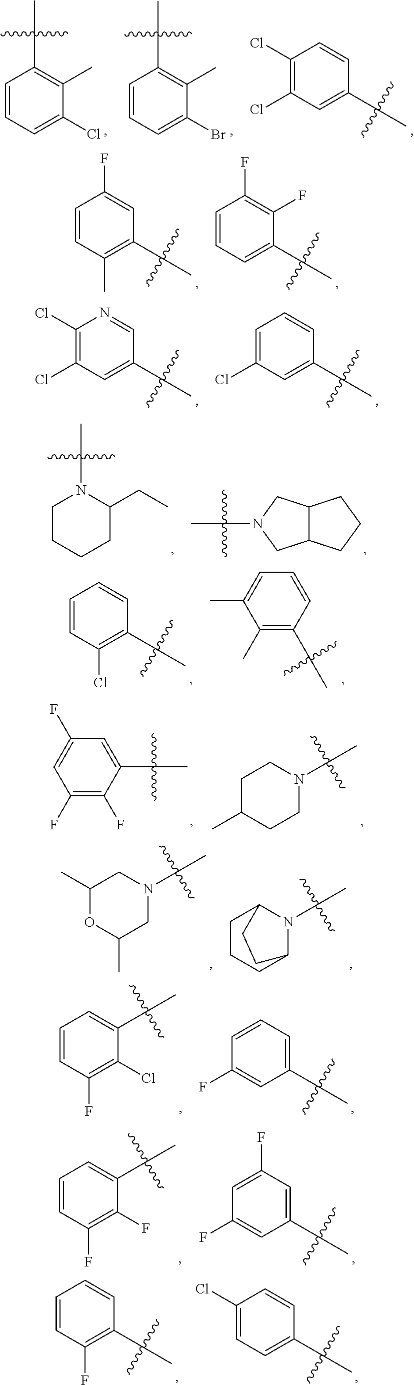

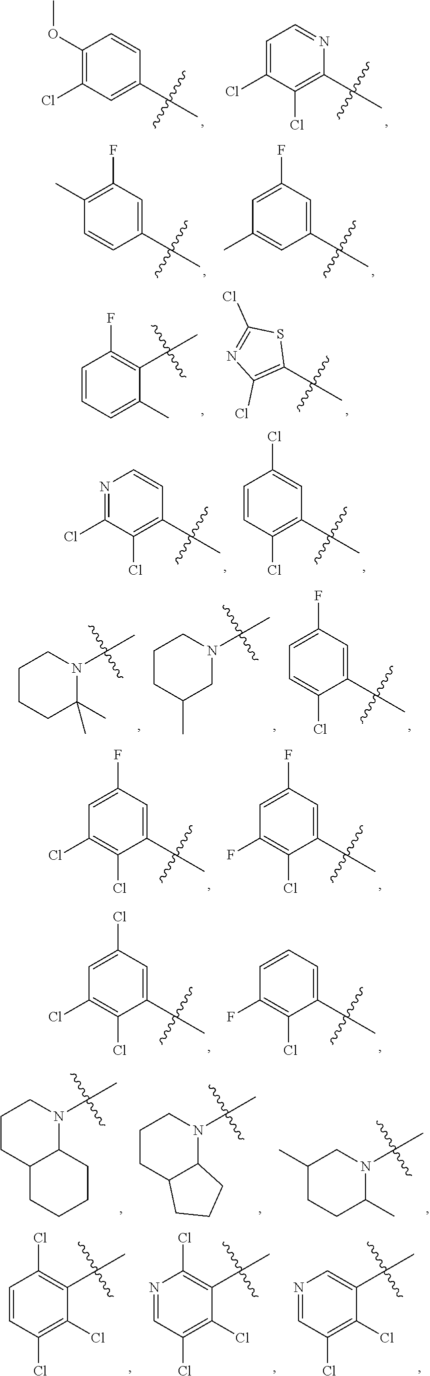

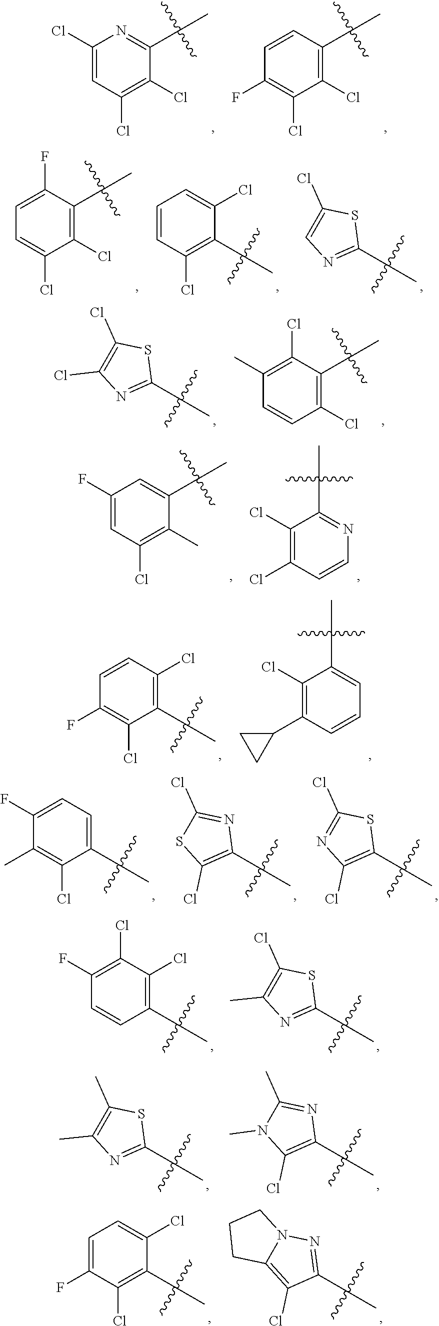

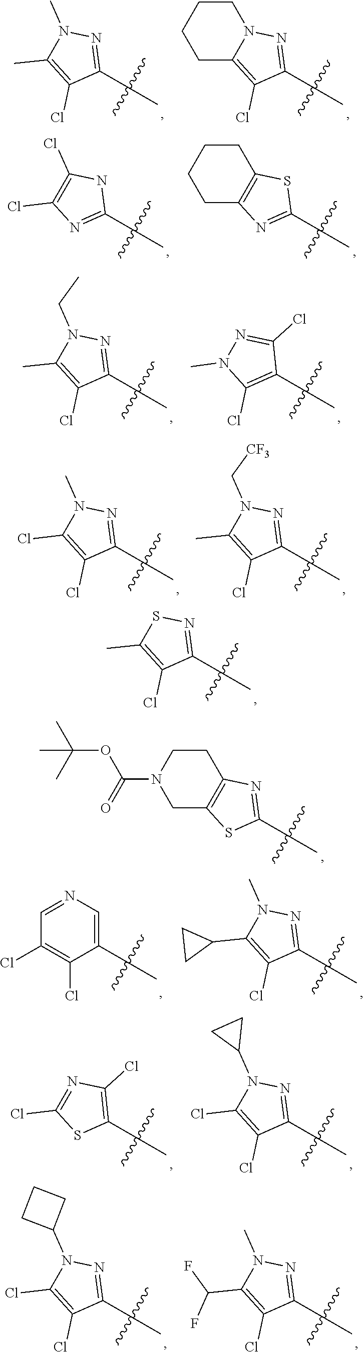

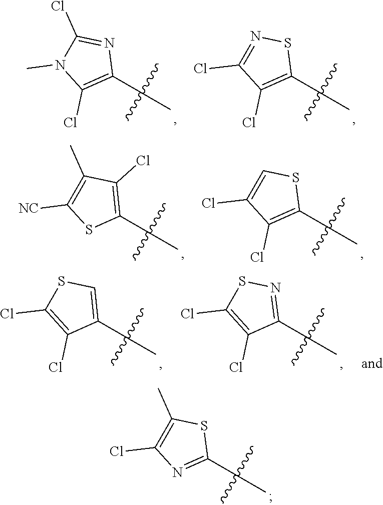

and all other variables not specifically defined herein are as defined in any one of the suitable preceding embodiments.

In a 48^{th} embodiment, in a compound, tautomer, a solvate or stereoisomer of the compound or the tautomer, or pharmaceutically acceptable salt of this disclosure, Ring A substituted with m groups of R^{a} is selected from:

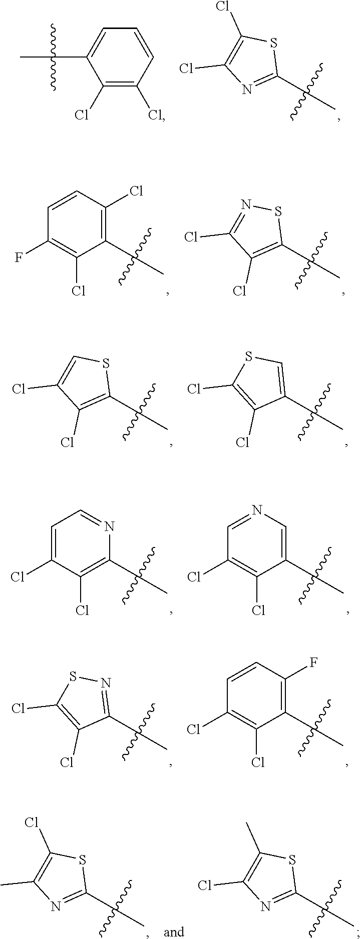

all other variables not specifically defined herein are as defined in any one of the suitable preceding embodiments.

In a 49^{th} embodiment, in a compound, tautomer, a solvate or stereoisomer of the compound or the tautomer, or pharmaceutically acceptable salt of this disclosure, Ring B substituted with n groups of R^{b} is:

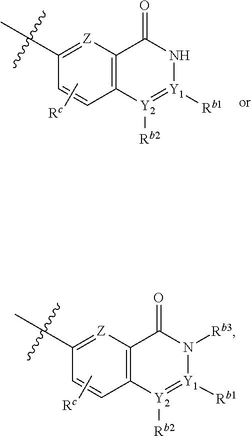

wherein Z is selected from C and N, Y₁ and Y₂ are independently selected from C and N, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{b3} is selected from H, methyl, ethyl,

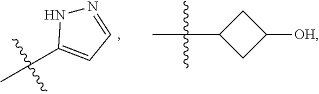 [SMILES: *c1ccn[nH]1 ; *C1CC(O)C1]

and 5-membered heteroaryl, wherein the 5-membered heteroaryl of R^{b3} contains 2 to 3 heteroatoms selected from N and S and the 5-membered heteroaryl is optionally substituted with 1-2 groups selected from halogen and C₁ to C₄ alkyl, and R^{c} is selected from H, halogen, CN, and methyl; and all other variables not specifically defined herein are as defined in any one of the suitable preceding embodiments.

In a 50^{th} embodiment, in a compound, tautomer, a solvate or stereoisomer of the compound or the tautomer, or pharmaceutically acceptable salt of this disclosure, Ring B substituted with n groups of R^{b} is selected from:

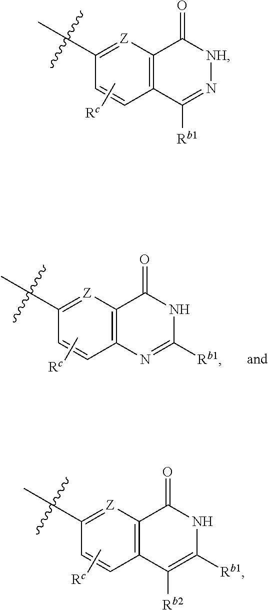

wherein Z is selected from C and N, R^{b1} is selected from H, F, Cl, Br, methyl, CN, CF₃, CF₂H, ethyl, cyclopropyl, and OCH₃, R^{b2} is selected from H, F, Cl, Br, methyl, CN, CF₃, CF₂H, ethyl, cyclopropyl, and OCH₃, and R^{c} is selected from H, halogen, CN, and methyl; and all other variables not specifically defined herein are as defined in any one of the suitable preceding embodiments.

In a 51^{st} embodiment, in a compound, tautomer, a solvate or stereoisomer of the compound or the tautomer, or pharmaceutically acceptable salt of this disclosure, Ring B substituted with n groups of R^{b} is:

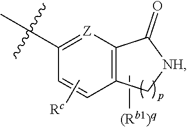

wherein Z is selected from C and N, R^{b1} is selected from F, Cl, Br, methyl, CN, CF₃, CF₂H, ethyl, cyclopropyl, and OCH₃, R^{c} is selected from H, halogen, CN, and methyl, p is 1, 2, or 3, and q is 0, 1, or 2; and all other variables not specifically defined herein are as defined in any one of the suitable preceding embodiments.

In a 52^{nd} embodiment, in a compound, tautomer, a solvate or stereoisomer of the compound or the tautomer, or pharmaceutically acceptable salt of this disclosure, Ring B substituted with n groups of R^{b} is selected from:

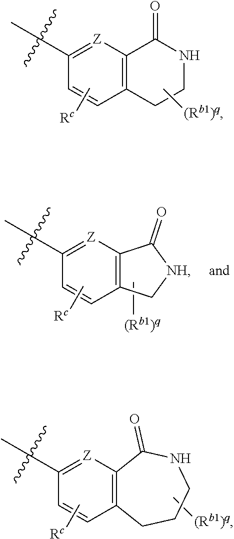

wherein Z is selected from C and N, R^{b1} is selected from F, Cl, Br, methyl, CN, CF₃, CF₂H, ethyl, cyclopropyl, and OCH₃, R^{c} is selected from H, halogen, CN, and methyl, and q is 0, 1, or 2; and all other variables not specifically defined herein are as defined in any one of the suitable preceding embodiments.

In a 53^{rd} embodiment, in a compound, tautomer, a solvate or stereoisomer of the compound or the tautomer, or pharmaceutically acceptable salt of this disclosure, Ring B substituted with n groups of R^{b} is:

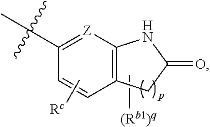

wherein Z is selected from C and N, R^{b1} selected from F, Cl, Br, methyl, CN, CF₃, CF₂H, ethyl, cyclopropyl, and OCH3, R^{c} is H, halogen, CN, or methyl, p is 1, 2 or 3, q is 0, 1, or 2; and all other variables not specifically defined herein are as defined in any one of the suitable preceding embodiments.

In a 54^{th} embodiment, in a compound, tautomer, a solvate or stereoisomer of the compound or the tautomer, or pharmaceutically acceptable salt of this disclosure, Ring B substituted with n groups of R^{b} is selected from:

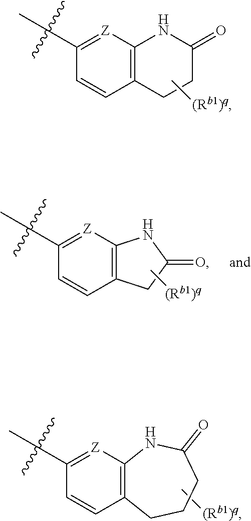

wherein Z is selected from C and N, R^{b1} selected from F, Cl, Br, methyl, CN, CF₃, CF₂H, ethyl, cyclopropyl, and OCH₃, and q is 1 or 2; and all other variables not specifically defined herein are as defined in any one of the suitable preceding embodiments.

In a 55^{th} embodiment, in a compound, tautomer, a solvate or stereoisomer of the compound or the tautomer, or pharmaceutically acceptable salt of this disclosure, Ring B substituted with n groups of R^{b} is

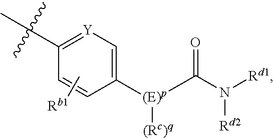

wherein Y is selected from C and N, E is selected from C, N, and O, R^{d1} and R^{d2} are independently selected from H, methyl, ethyl, and cyclopropyl, R^{c} is selected from F, Cl, Br, methyl, CN, CF₃, CF₂H, ethyl, cyclopropyl, and OCH₃, R^{b1} is selected from H, halogen, CN, and methyl, p is 1 or 2, and q is 0, 1, or 2; and all other variables not specifically defined herein are as defined in any one of the suitable preceding embodiments.

In a 56^{th} embodiment, in a compound, tautomer, a solvate or stereoisomer of the compound or the tautomer, or pharmaceutically acceptable salt of this disclosure, Ring B substituted with n groups of R^{b} is

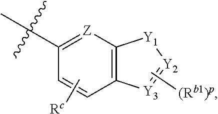

wherein Z is selected from C and N, Y₁, Y₂, and Y₃ are independently selected from C, N, S, and O, R^{b1} is selected from F, Cl, Br, methyl, CN, CF₃, CF₂H, ethyl, cyclopropyl, CONH₂, OCH₃, CH₂CONH₂,

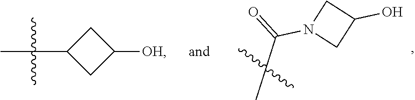 [SMILES: *C1CC(O)C1 ; "and" ; *C(=O)N1CC(O)C1 ; ","]

R^{c} is selected from H, halogen, CN, and methyl, and p is 0, 1, or 2; and all other variables not specifically defined herein are as defined in any one of the suitable preceding embodiments.

In a 57^{th} embodiment, in a compound, tautomer, a solvate or stereoisomer of the compound or the tautomer, or pharmaceutically acceptable salt of this disclosure, Ring B substituted with n groups of R^{b} is selected from

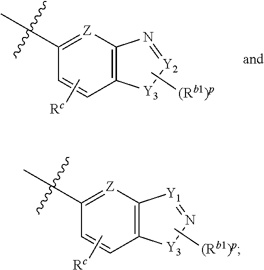

and all other variables not specifically defined herein are as defined in any one of the suitable preceding embodiments.

In a 58^{th} embodiment, in a compound, tautomer, a solvate or stereoisomer of the compound or the tautomer, or pharmaceutically acceptable salt of this disclosure, Ring B substituted with n groups of R^{b} is

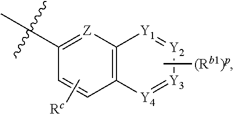

Z is selected from C and N, Y₁, Y₂, Y₃, and Y₄ are independently selected from C and N, R^{b1} is selected from F, Cl, Br, methyl, CN, CF₃, CF₂H, ethyl, cyclopropyl, CONH₂, and OCH₃, R^{c} is H, halogen, CN, or methyl, and p is 0, 1, or 2; and all other variables not specifically defined herein are as defined in any one of the suitable preceding embodiments.

In a 59^{th} embodiment, in a compound, tautomer, a solvate or stereoisomer of the compound or the tautomer, or pharmaceutically acceptable salt of this disclosure, Ring B is selected from:

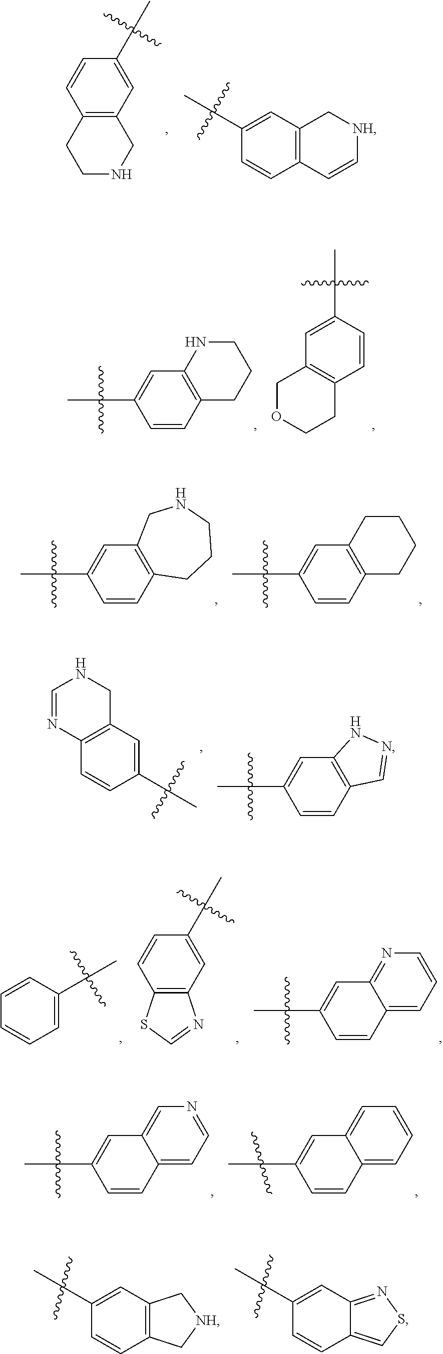

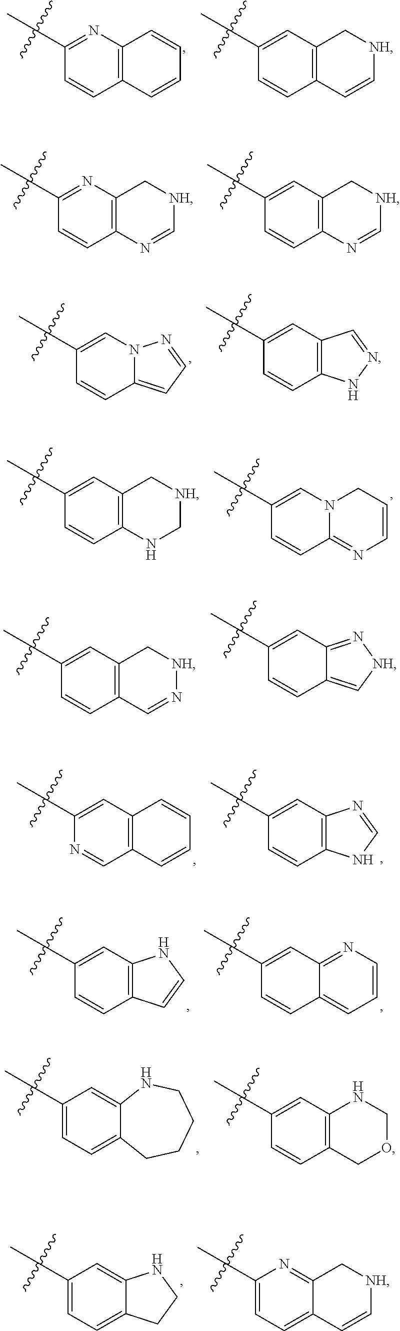

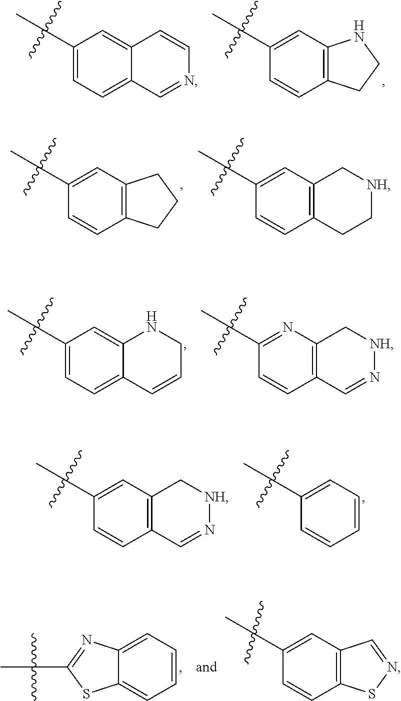

wherein Ring B is substituted with n groups of R^{b}; and all other variables not specifically defined herein are as defined in any one of the suitable preceding embodiments.

In a 60^{th} embodiment, in a compound, tautomer, a solvate or stereoisomer of the compound or the tautomer, or pharmaceutically acceptable salt of this disclosure, Ring B substituted with n groups of R^{b} is selected from:

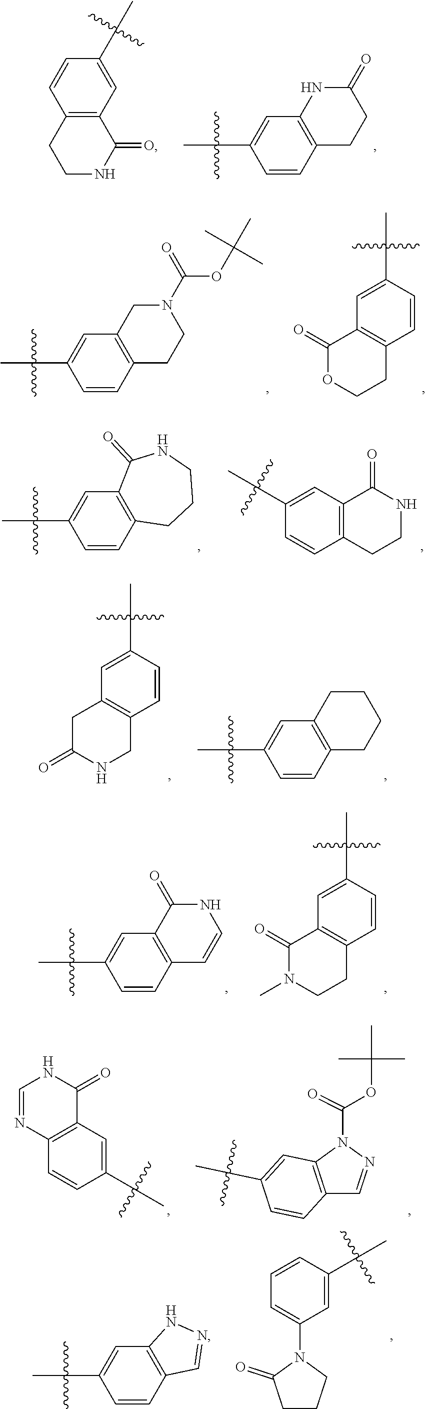

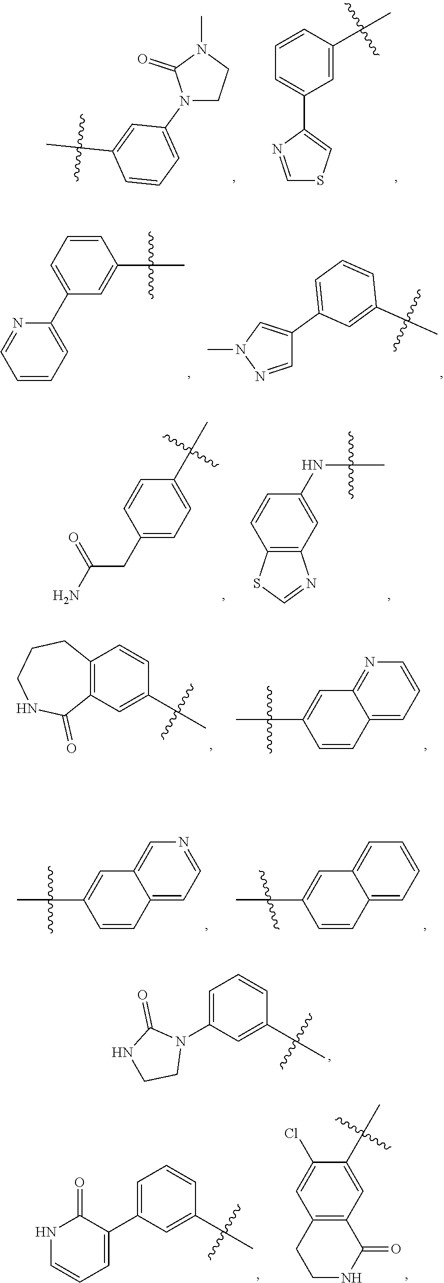

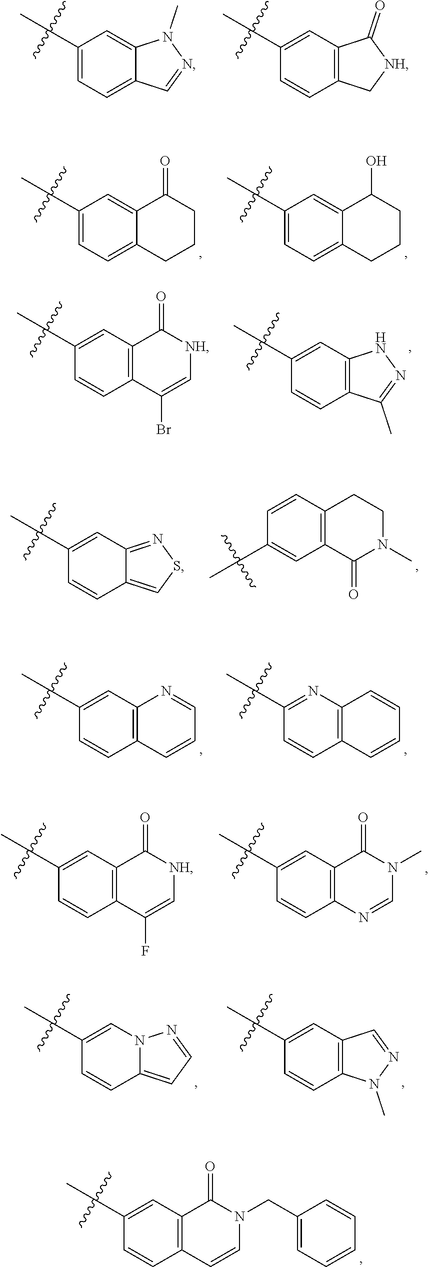

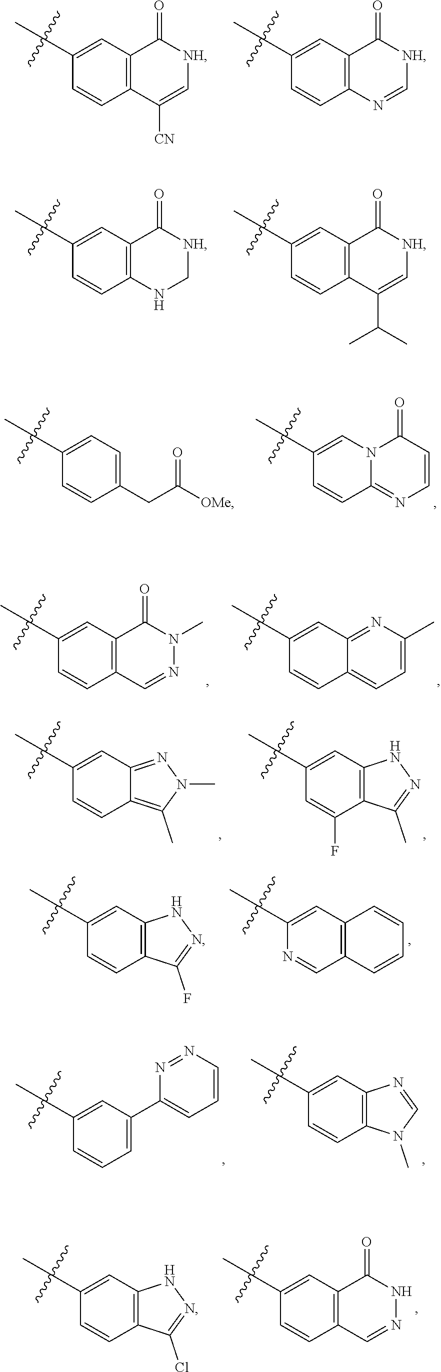

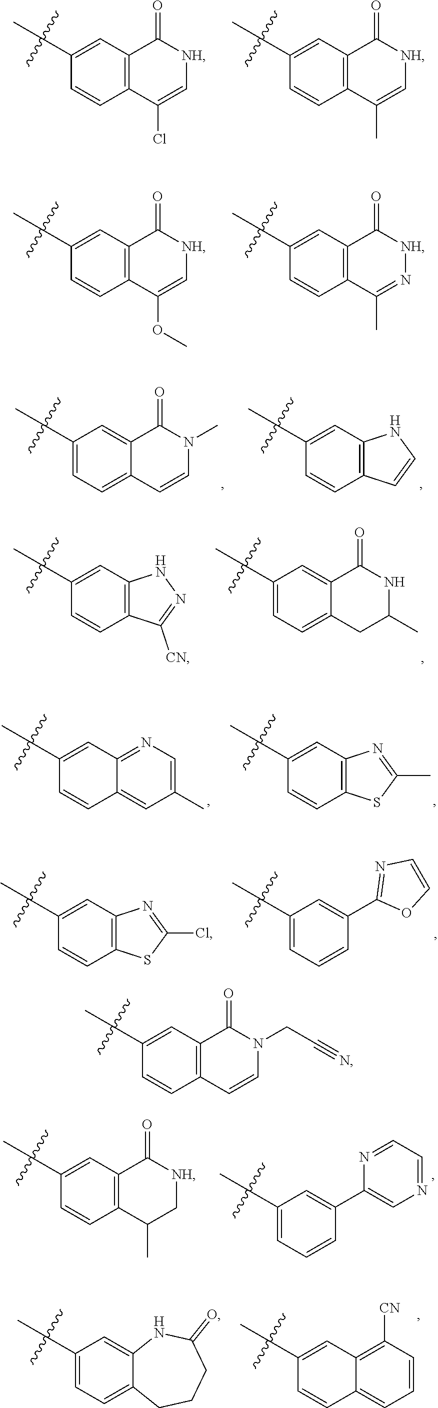

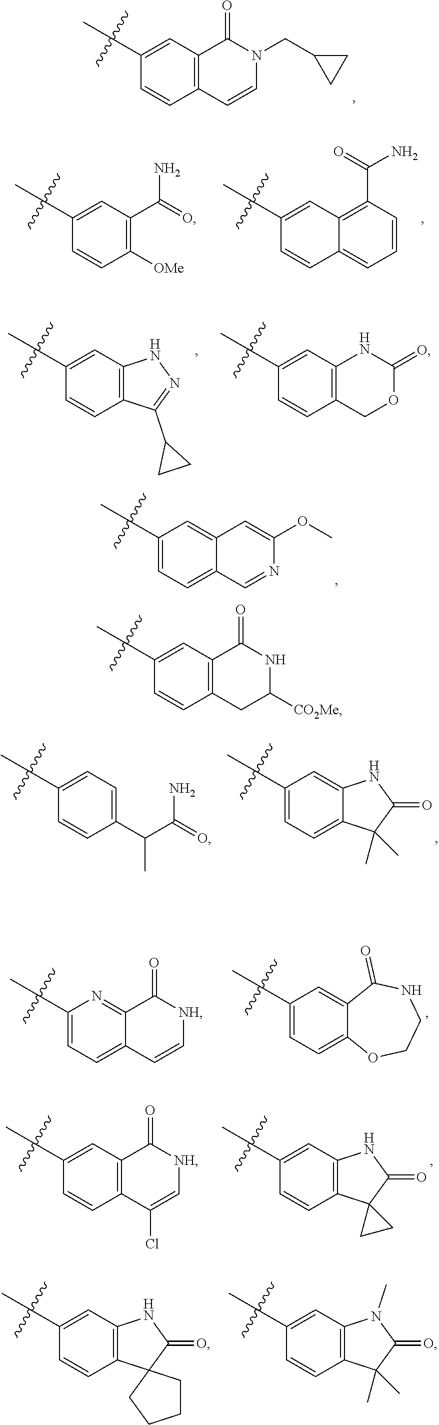

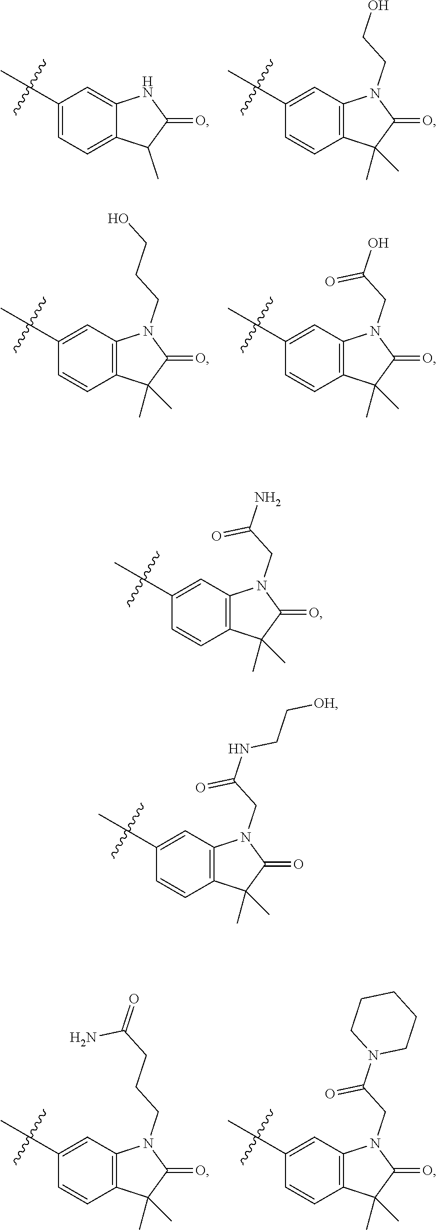

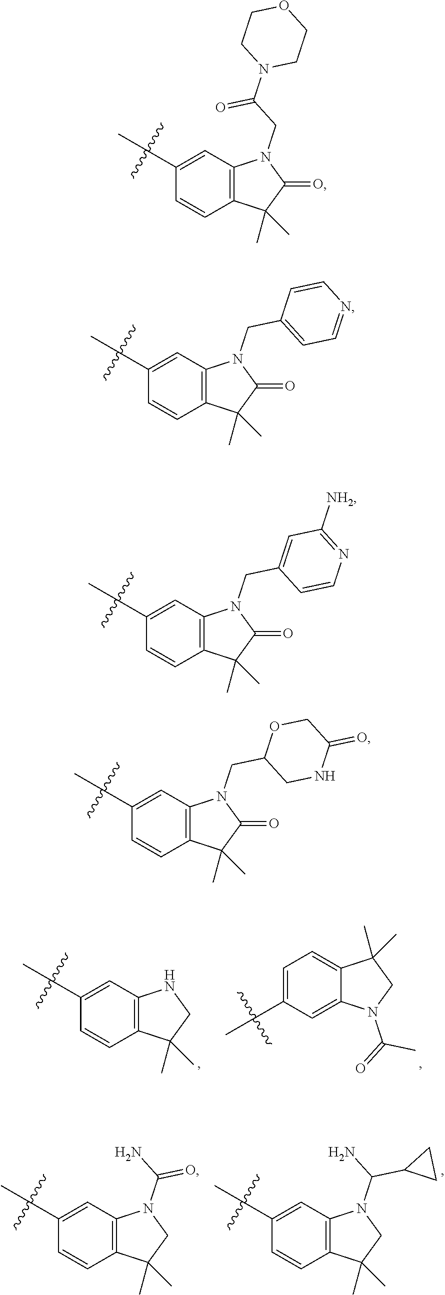

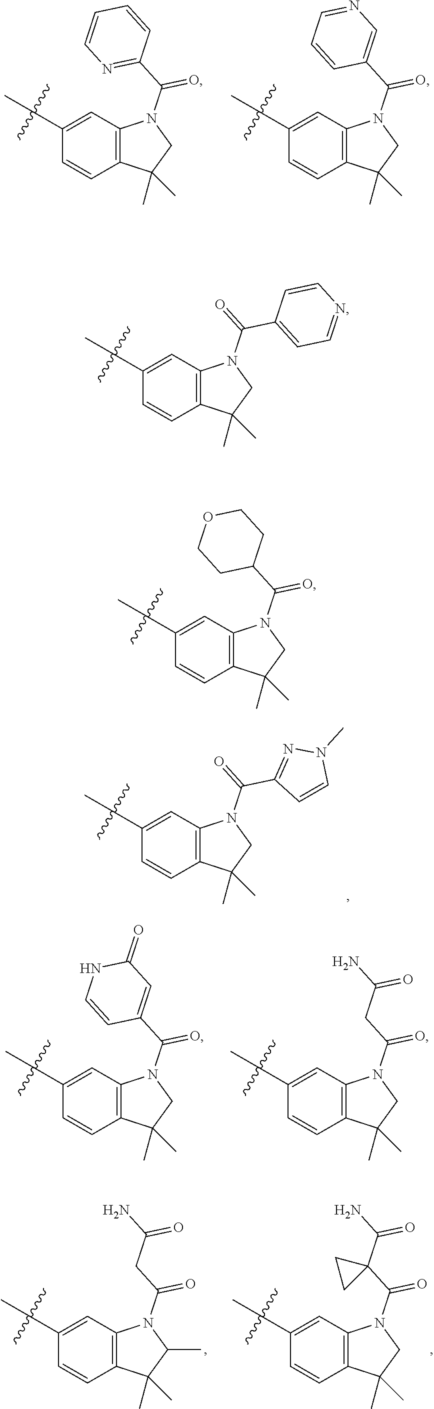

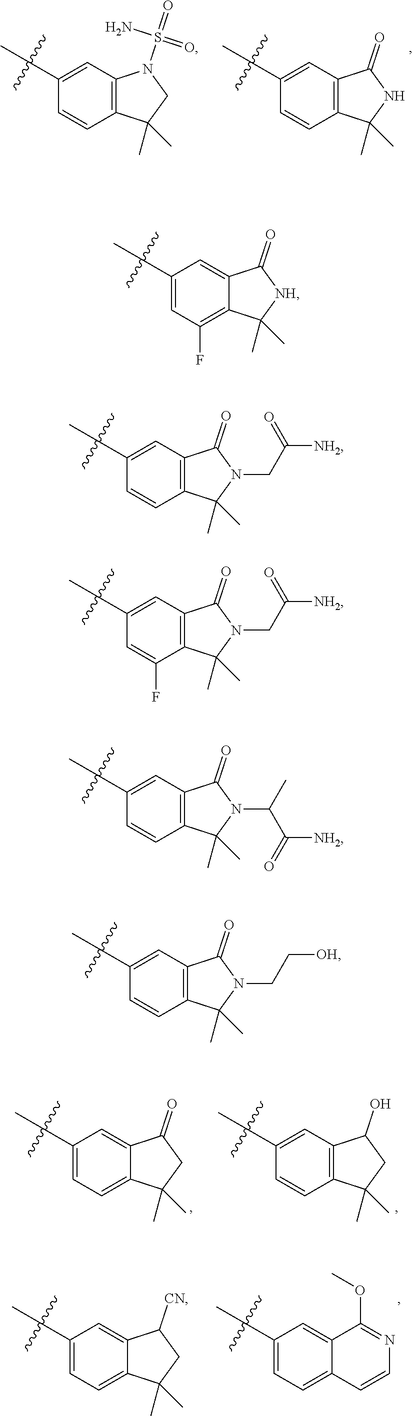

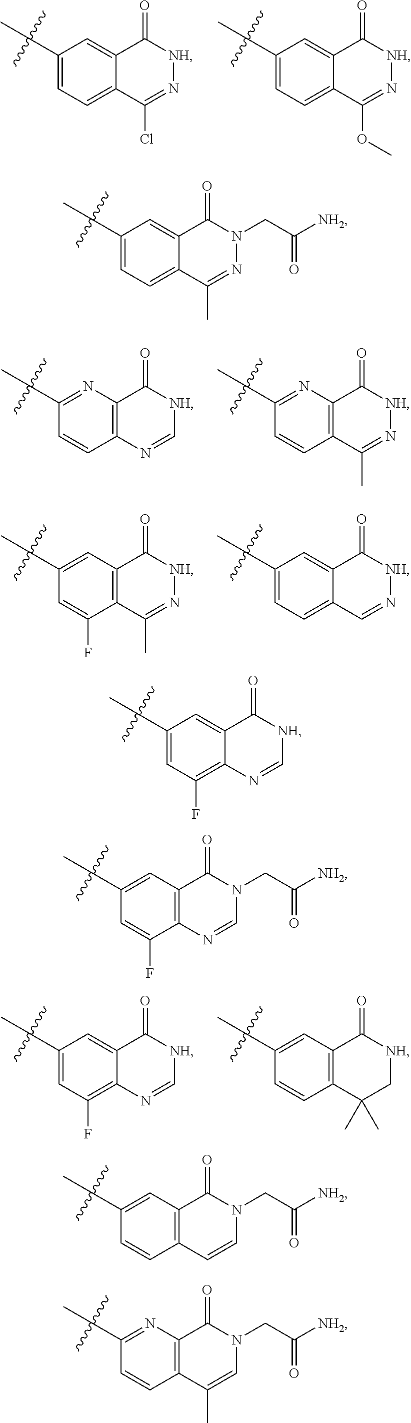

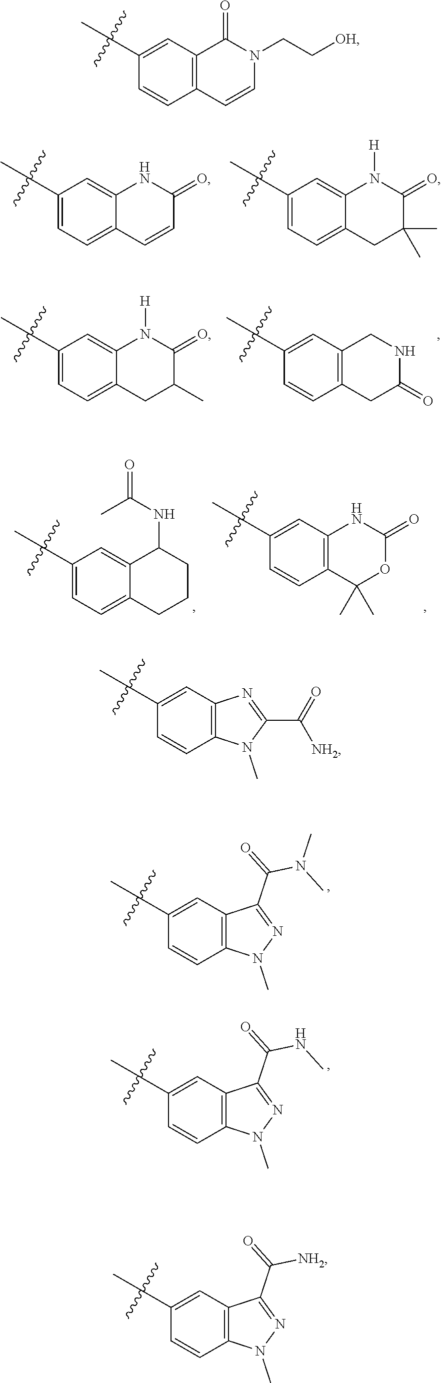

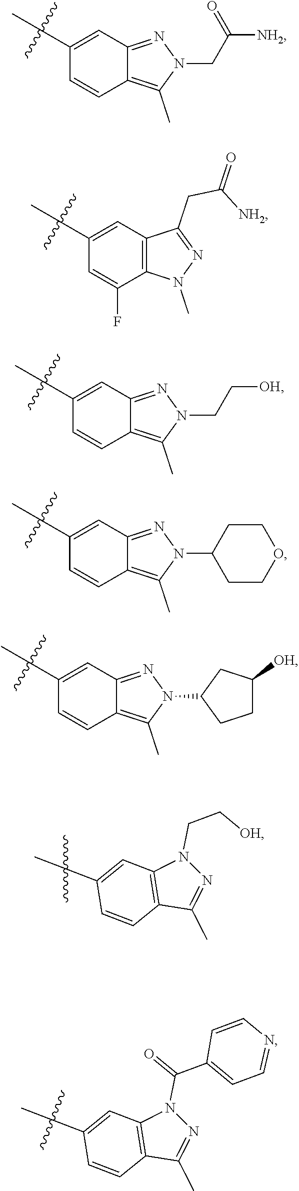

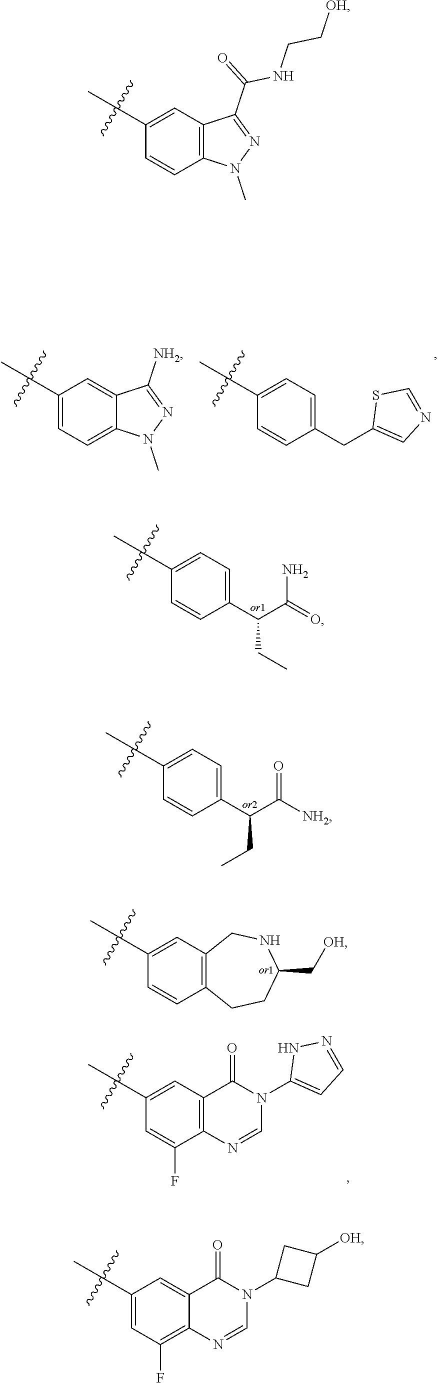

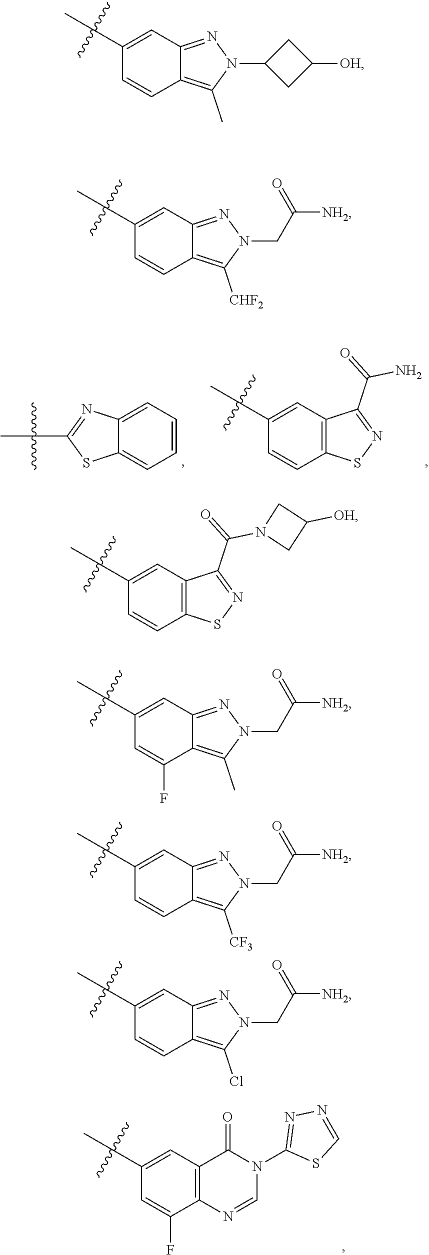

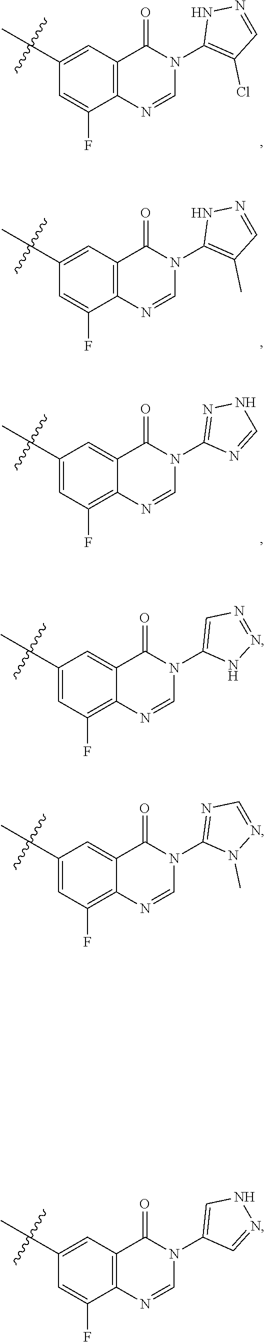 [SMILES: *c1cc(F)c2ncn(-c3[nH]ncc3Cl)c(=O)c2c1 ; *c1cc(F)c2ncn(-c3[nH]ncc3C)c(=O)c2c1 ; *c1cc(F)c2ncn(-c3nc[nH]n3)c(=O)c2c1 ; *c1cc(F)c2ncn(-c3cnn[nH]3)c(=O)c2c1 ; *c1cc(F)c2ncn(-c3ncnn3C)c(=O)c2c1 ; *c1cc(F)c2ncn(-c3cn[nH]c3)c(=O)c2c1]

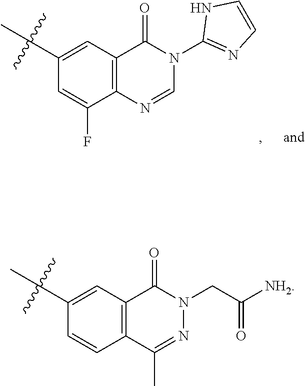 [SMILES: *c1cc(F)c2ncn(-c3ncc[nH]3)c(=O)c2c1 ; *c1ccc2c(C)nn(CC(N)=O)c(=O)c2c1]

and all other variables not specifically defined herein are as defined in any one of the suitable preceding embodiments.

In a 61^{st} embodiment, in a compound, tautomer, a solvate or stereoisomer of the compound or the tautomer, or pharmaceutically acceptable salt of this disclosure, Ring B substituted with n groups of R^{b} is selected from:

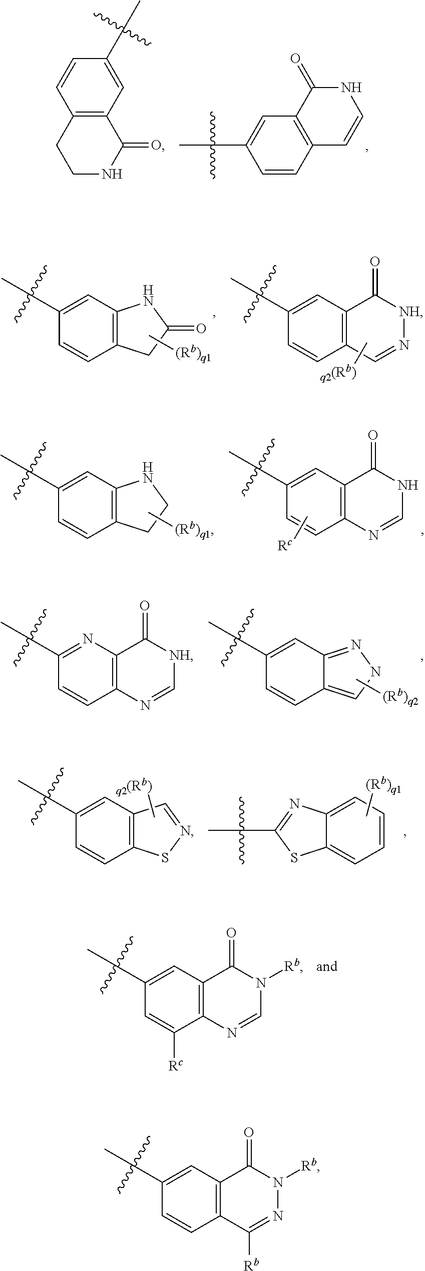

wherein q1 is an integer selected from 0, 1, 2, and 3, q2 is an integer selected from 0, 1, and 2, and R^{c} is selected from F, Cl, Brand Me; and all other variables not specifically defined herein are as defined in any one of the suitable preceding embodiments.

In a 62^{nd} embodiment, in a compound, tautomer, a solvate or stereoisomer of the compound or the tautomer, or pharmaceutically acceptable salt of this disclosure, wherein R^{a} is selected from C₃-C₅ cycloalkyl, —O(C₁-C₂ alkyl), COOH, CN, CONH₂, —C(═O)NH(C₁-C₂ alkyl), —C(═O)N(C₁-C₂ alkyl)₂, —C(═O)O(C₁-C₂ alkyl), halogen, and C₁-C₂ alkyl optionally substituted with 1 to 3 groups selected from halogen; and all other variables not specifically defined herein are as defined in any one of the suitable preceding embodiments.

In a 63^{rd} embodiment, in a compound, tautomer, a solvate or stereoisomer of the compound or the tautomer, or pharmaceutically acceptable salt of this disclosure, R is selected from CH₃, CH₂CH₃, CH₂CF₃, F, Cl, Br, C₃-C₄ cycloalkyl, COOH, CN, CONH₂, —C(═O)NHCH₃, —C(═O)N(CH₃)₂, —NC(═O)N(CH₃)₃, and OCH₃; and all other variables not specifically defined herein are as defined in any one of the suitable preceding embodiments.

In a 64^{th} embodiment, in a compound, tautomer, a solvate or stereoisomer of the compound or the tautomer, or pharmaceutically acceptable salt of this disclosure, R^{b} is selected from ═O, OH, halogen, CN, —C(═O)NH(C₁-C₄ alkyl), —C(═O)O(C₁-C₄ alkyl), —C(═O)NH₂,
C₃-C₄ cycloalkyl optionally substituted with OH,
O(C₁-C₃ alkyl), —C(═O)(C₁-C₂ alkyl), —C(═O)(C₃-C₆ cycloalkyl), —C(═O)(4- to 6-membered heterocyclyl optionally substituted with ═O), —C(═O)(5- to 6-membered heteroaryl optionally substituted with C₁-C₂ alkyl or NH₂), —C(═O)(CH₂)pC(═O)NH₂, —NHC(═O)(C₁-C₂ alkyl), —C(═O)N(C₁-C₂ alkyl)₂, —C(═O)NH(CH₂)pOH, —S(═O)₂NH₂,

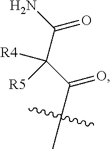

C₁-C₃ alkyl optionally substituted with 1 to 3 groups selected from halogen, NH₂, OH, OCH₃, —C(═O)NH(C₁-C₄ alkyl), —C(═O)O(C₁-C₄ alkyl), —C(═O)N(C₁-C₄ alkyl)₂, —COOH, —C(═O)NH₂, CN, C₃-C₄ cycloalkyl, phenyl, —C(═O)(C₁-C₂ alkyl), —C(═O)(C₃-C₆ cycloalkyl), —C(═O)(5- to 6-membered heterocyclyl), —C(═O)(5- to 6-membered heteroaryl optionally substituted with C₁-C₂ alkyl), C(═O)NH(CH₂)pOH, and 5- to 6-membered heteroaryl, 5- to 6-membered heteroaryl optionally substituted with 1 to 2 groups selected from C₁-C₂ alkyl, and
5- to 6-membered heterocyclyl optionally substituted with 1 to 2 groups selected from ═O and C₁-C₂ alkyl, or
two R^{b} attached to the same position on Ring B join to form a C₃-C₅ cycloalkyl,
wherein p is an integer selected from 1 and 2, and
wherein R₄ and R₅ are each independently selected from H and C₁ to C₃ alkyl or R₄ and R₅ join to form a 3 to 5-membered cycloalkyl;
and all other variables not specifically defined herein are as defined in any one of the suitable preceding embodiments.

In a 65^{th} embodiment, in a compound, tautomer, a solvate or stereoisomer of the compound or the tautomer, or pharmaceutically acceptable salt of this disclosure, R^{b} is selected from ═O,

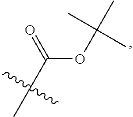 [SMILES: *C(=O)OC(C)(C)C]

CH₃, CHF₂, CF₃, OH, F, Cl, Br, CN, CH(CH₃)₂, CH₂CN, OCH₃, —C(═O)NH₂, —C(═O)OCH₃,

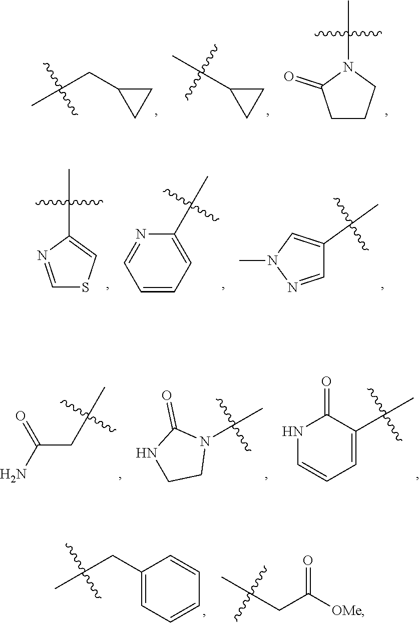

CH₂CH₂H, CH₂CH₂CH₂OH, CH₂C(═O)OH, CH₂C(═O)NH₂, CH₂C(═O)NHCH₂CH₂OH, CH₂CH₂CH₂C(═O)NH₂,

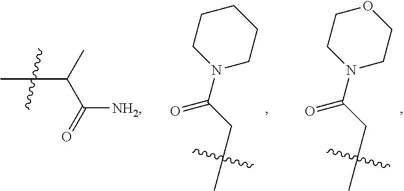

### —C(═O)CH₃,

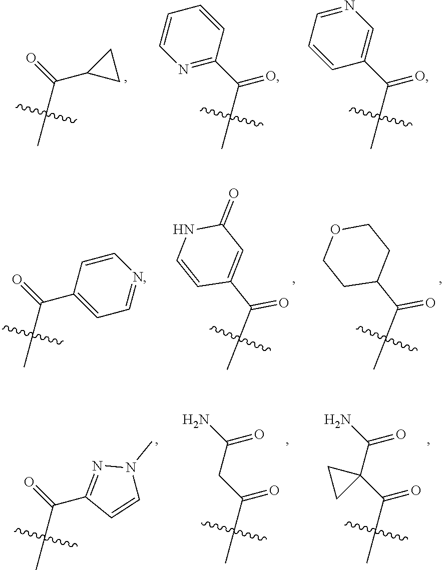

—NHC(═O)CH₃, —C(═O)N(CH₃)₂, —C(═O)NCH₃, NH₂, —C(═O)NHCH₂CH₂OH

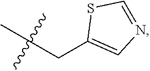 [SMILES: *Cc1cncs1]

### CH₂OH,

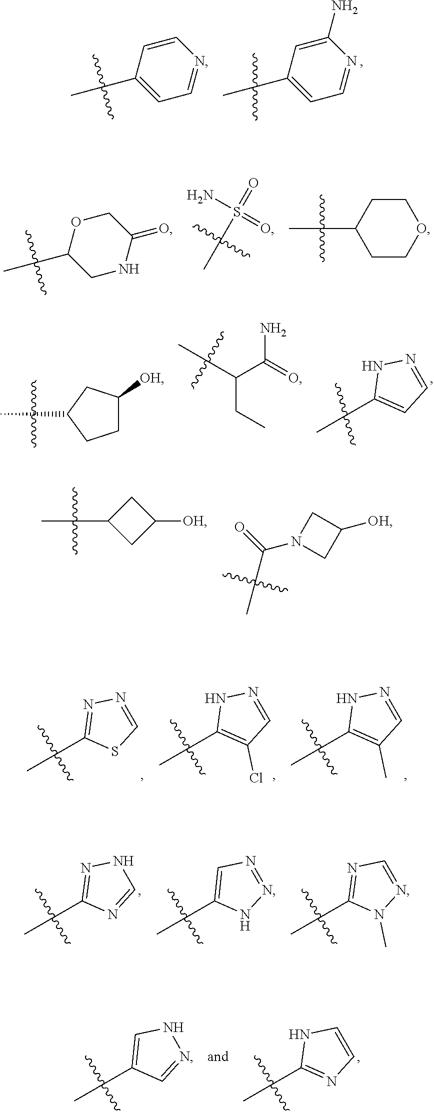

or two R^{b} attached to the same position on Ring B join to form a 3-membered or 5-membered cycloalkyl; and all other variables not specifically defined herein are as defined in any one of the suitable preceding embodiments.

In a 66^{th} embodiment, in a compound, tautomer, a solvate or stereoisomer of the compound or the tautomer, or pharmaceutically acceptable salt of this disclosure, L is selected from

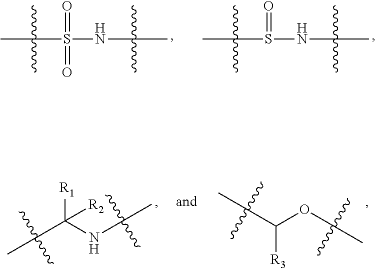

wherein
R₁ is H, D, or C₁-C₄ alkyl, and R₂ is selected from CN, C₁-C₄ alkenyl (e.g., C₂, C₃, or C₄ alkenyl), C₃-C₅ cycloalkyl, 5 to 6-membered heteroaryl,
   phenyl optionally substituted with 1 to 2 groups selected from C₁-C₃ alkyl (e.g., C₁, C₂, or C₃ alkyl) and halogen, and
   C₁-C₄ alkyl (e.g., C₁, C₂, C₃ or C₄ alkyl) optionally substituted with 1 to 3 groups
selected from halo, OH, and phenyl,
or
R₁ and R₂ join to form a 3-4 membered heterocyclyl; and
R₃ is C₁-C₄ alkyl (e.g., C₁, C₂, C₃ or C₄ alkyl) optionally substituted with 1 to 3 groups selected from halogen and OH; and all other variables not specifically defined herein are as defined in any one of the suitable preceding embodiments.

In a 67^{th} embodiment, in a compound, tautomer, a solvate or stereoisomer of the compound or the tautomer, or pharmaceutically acceptable salt of this disclosure, L is selected from:

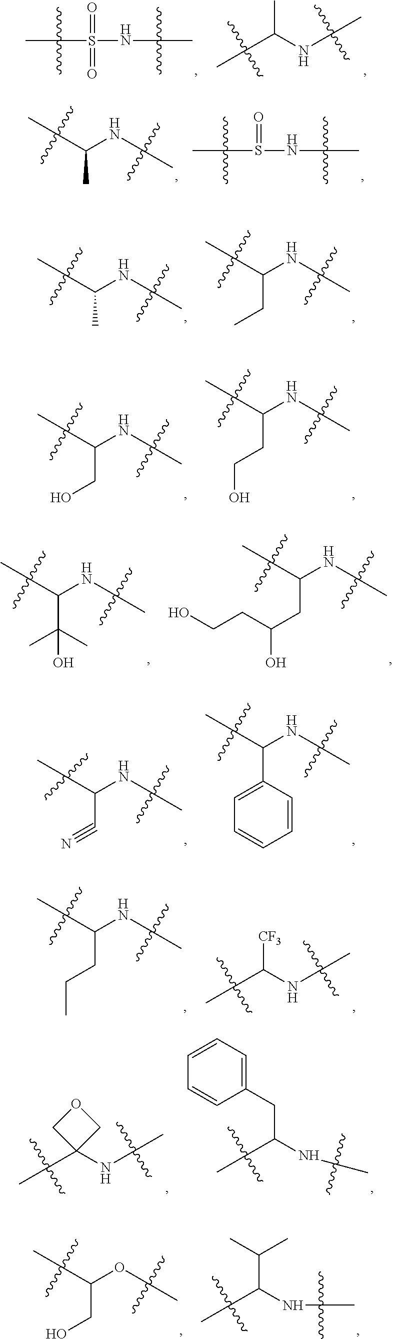

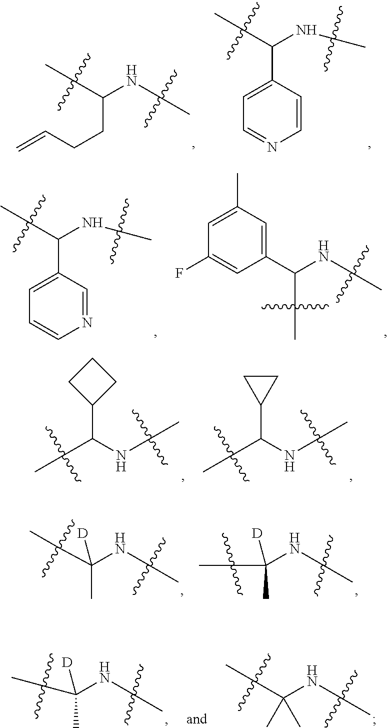

and all other variables not specifically defined herein are as defined in any one of the suitable preceding embodiments.

In a 68^{th} embodiment, in a compound, tautomer, a solvate or stereoisomer of the compound or the tautomer, or pharmaceutically acceptable salt of this disclosure, L is selected from

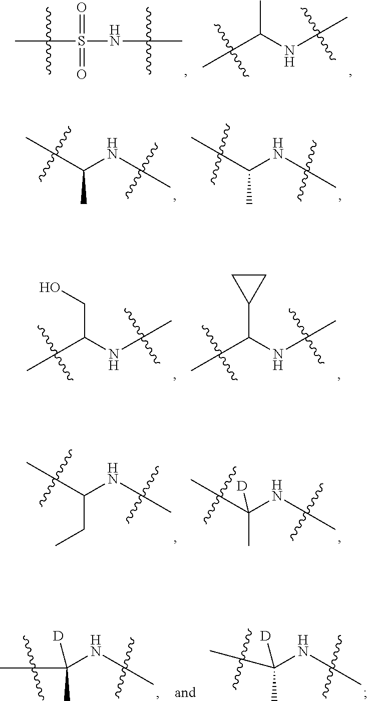

and all other variables not specifically defined herein are as defined in any one of the suitable preceding embodiments.

In certain embodiments, a compound of the disclosure is selected from Compounds 1 to 391 depicted in Table 1, a tautomer thereof, a solvate or stereoisomer of the compound or the tautomer, or a pharmaceutically acceptable salt of the foregoing.

TABLE 1
Compounds 1 to 391

| | |
|---|---|
| 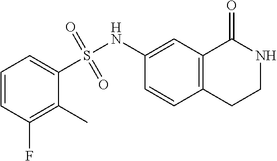 [SMILES: Cc1c(F)cccc1S(=O)(=O)Nc1ccc2c(c1)C(=O)NCC2] | 1 |
| 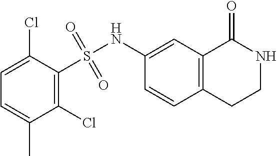 [SMILES: Cc1ccc(Cl)c(S(=O)(=O)Nc2ccc3c(c2)C(=O)NCC3)c1Cl] | 2 |
| 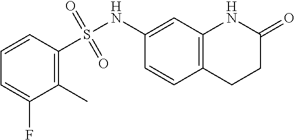 [SMILES: Cc1c(F)cccc1S(=O)(=O)Nc1ccc2c(c1)NC(=O)CC2] | 3 |
| 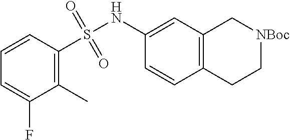 [SMILES: Cc1c(F)cccc1S(=O)(=O)Nc1ccc2c(c1)CN(C(=O)OC(C)(C)C)CC2] | 4 |
| 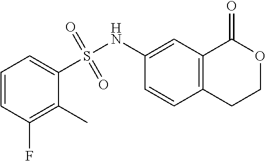 [SMILES: Cc1c(F)cccc1S(=O)(=O)Nc1ccc2c(c1)C(=O)OCC2] | 5 |
| 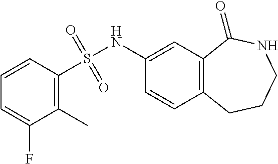 [SMILES: Cc1c(F)cccc1S(=O)(=O)Nc1ccc2c(c1)C(=O)NCCC2] | 6 |
| 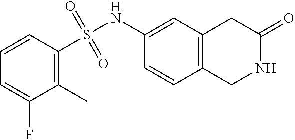 [SMILES: Cc1c(F)cccc1S(=O)(=O)Nc1ccc2c(c1)CC(=O)NC2] | 7 |
| 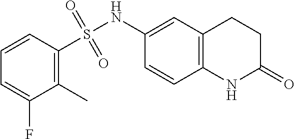 [SMILES: Cc1c(F)cccc1S(=O)(=O)Nc1ccc2c(c1)CCC(=O)N2] | 8 |
| 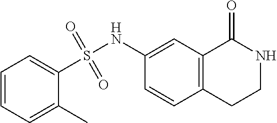 [SMILES: Cc1ccccc1S(=O)(=O)Nc1ccc2c(c1)C(=O)NCC2] | 9 |
| 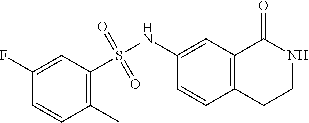 [SMILES: Cc1ccc(F)cc1S(=O)(=O)Nc1ccc2c(c1)C(=O)NCC2] | 10 |
| 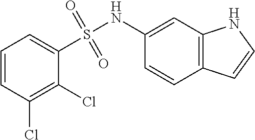 [SMILES: O=S(=O)(Nc1ccc2cc[nH]c2c1)c1cccc(Cl)c1Cl] | 11 |
| 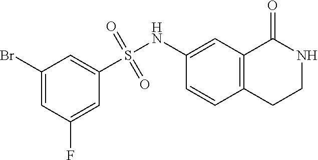 [SMILES: O=C1NCCc2ccc(NS(=O)(=O)c3cc(F)cc(Br)c3)cc21] | 12 |
| 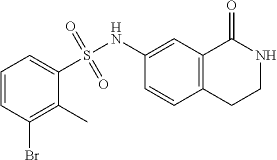 [SMILES: Cc1c(Br)cccc1S(=O)(=O)Nc1ccc2c(c1)C(=O)NCC2] | 13 |
| 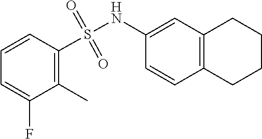 [SMILES: Cc1c(F)cccc1S(=O)(=O)Nc1ccc2c(c1)CCCC2] | 14 |
| 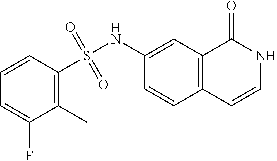 [SMILES: Cc1c(F)cccc1S(=O)(=O)Nc1ccc2cc[nH]c(=O)c2c1] | 15 |
| 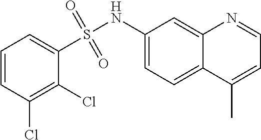 [SMILES: Cc1ccnc2cc(NS(=O)(=O)c3cccc(Cl)c3Cl)ccc12] | 16 |
| 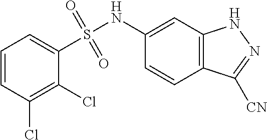 [SMILES: N#Cc1n[nH]c2cc(NS(=O)(=O)c3cccc(Cl)c3Cl)ccc12] | 17 |
| 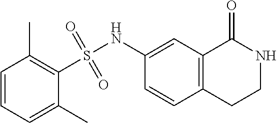 [SMILES: Cc1cccc(C)c1S(=O)(=O)Nc1ccc2c(c1)C(=O)NCC2] | 18 |
| 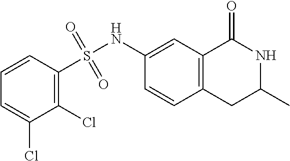 [SMILES: CC1Cc2ccc(NS(=O)(=O)c3cccc(Cl)c3Cl)cc2C(=O)N1] | 19 |
| 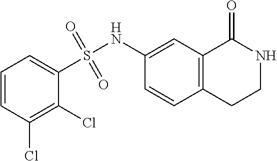 [SMILES: O=C1NCCc2ccc(NS(=O)(=O)c3cccc(Cl)c3Cl)cc21] | 20 |
| 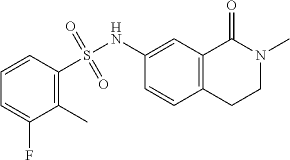 [SMILES: Cc1c(F)cccc1S(=O)(=O)Nc1ccc2c(c1)C(=O)N(C)CC2] | 21 |
| 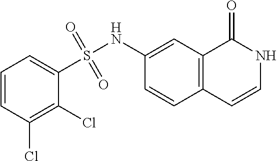 [SMILES: O=c1[nH]ccc2ccc(NS(=O)(=O)c3cccc(Cl)c3Cl)cc12] | 22 |
| 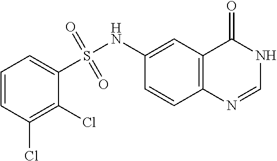 [SMILES: O=c1[nH]cnc2ccc(NS(=O)(=O)c3cccc(Cl)c3Cl)cc12] | 23 |
| 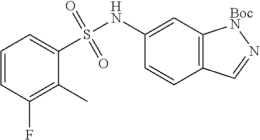 [SMILES: Cc1c(F)cccc1S(=O)(=O)Nc1ccc2cnn(C(=O)OC(C)(C)C)c2c1] | 24 |
| 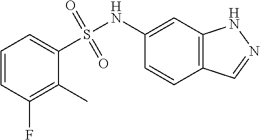 [SMILES: Cc1c(F)cccc1S(=O)(=O)Nc1ccc2cn[nH]c2c1] | 25 |
| 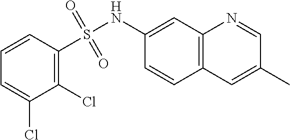 [SMILES: Cc1cnc2cc(NS(=O)(=O)c3cccc(Cl)c3Cl)ccc2c1] | 26 |
| 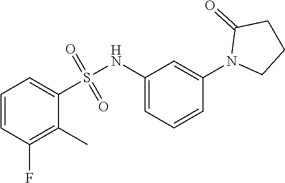 [SMILES: Cc1c(F)cccc1S(=O)(=O)Nc1cccc(N2CCCC2=O)c1] | 27 |
| 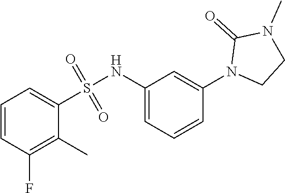 [SMILES: Cc1c(F)cccc1S(=O)(=O)Nc1cccc(N2CCN(C)C2=O)c1] | 28 |
| 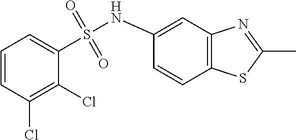 [SMILES: Cc1nc2cc(NS(=O)(=O)c3cccc(Cl)c3Cl)ccc2s1] | 29 |
| 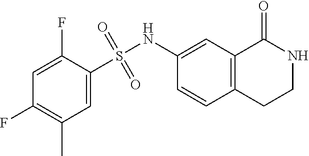 [SMILES: Cc1cc(S(=O)(=O)Nc2ccc3c(c2)C(=O)NCC3)c(F)cc1F] | 30 |
| 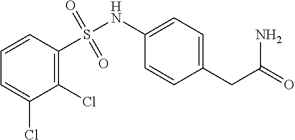 [SMILES: NC(=O)Cc1ccc(NS(=O)(=O)c2cccc(Cl)c2Cl)cc1] | 31 |
| 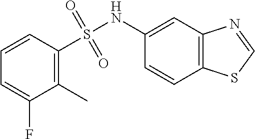 [SMILES: Cc1c(F)cccc1S(=O)(=O)Nc1ccc2scnc2c1] | 32 |
| 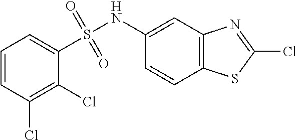 [SMILES: O=S(=O)(Nc1ccc2sc(Cl)nc2c1)c1cccc(Cl)c1Cl] | 33 |
| 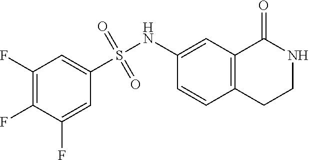 [SMILES: O=C1NCCc2ccc(NS(=O)(=O)c3cc(F)c(F)c(F)c3)cc21] | 34 |
| 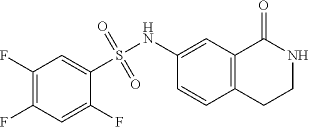 [SMILES: O=C1NCCc2ccc(NS(=O)(=O)c3cc(F)c(F)cc3F)cc21] | 35 |
| 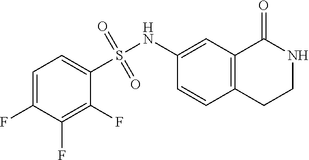 [SMILES: O=C1NCCc2ccc(NS(=O)(=O)c3ccc(F)c(F)c3F)cc21] | 36 |
| 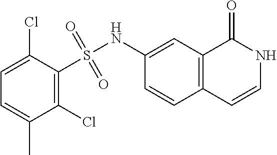 [SMILES: Cc1ccc(Cl)c(S(=O)(=O)Nc2ccc3cc[nH]c(=O)c3c2)c1Cl] | 37 |
| 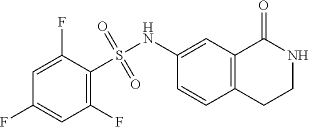 [SMILES: O=C1NCCc2ccc(NS(=O)(=O)c3c(F)cc(F)cc3F)cc21] | 38 |
| 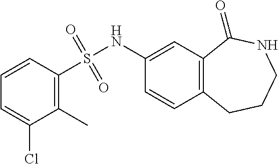 [SMILES: Cc1c(Cl)cccc1S(=O)(=O)Nc1ccc2c(c1)C(=O)NCCC2] | 39 |
| 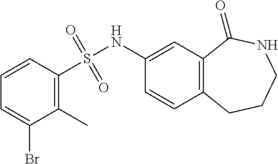 [SMILES: Cc1c(Br)cccc1S(=O)(=O)Nc1ccc2c(c1)C(=O)NCCC2] | 40 |
| 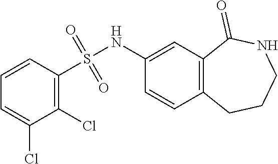 [SMILES: O=C1NCCCc2ccc(NS(=O)(=O)c3cccc(Cl)c3Cl)cc21] | 41 |
| 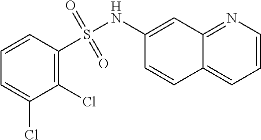 [SMILES: O=S(=O)(Nc1ccc2cccnc2c1)c1cccc(Cl)c1Cl] | 42 |
| 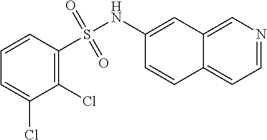 [SMILES: O=S(=O)(Nc1ccc2ccncc2c1)c1cccc(Cl)c1Cl] | 43 |
| 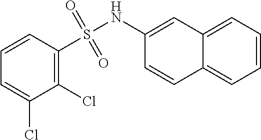 [SMILES: O=S(=O)(Nc1ccc2ccccc2c1)c1cccc(Cl)c1Cl] | 44 |
| 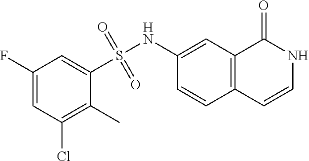 [SMILES: Cc1c(Cl)cc(F)cc1S(=O)(=O)Nc1ccc2cc[nH]c(=O)c2c1] | 45 |
| 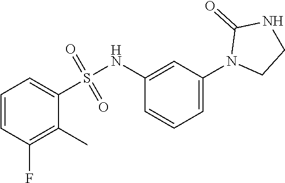 [SMILES: Cc1c(F)cccc1S(=O)(=O)Nc1cccc(N2CCNC2=O)c1] | 46 |
| 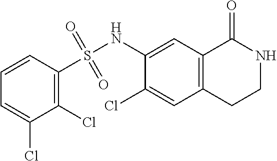 [SMILES: O=C1NCCc2cc(Cl)c(NS(=O)(=O)c3cccc(Cl)c3Cl)cc21] | 47 |
| 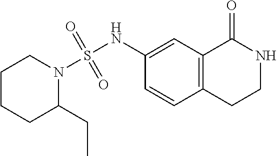 [SMILES: CCC1CCCCN1S(=O)(=O)Nc1ccc2c(c1)C(=O)NCC2] | 48 |
| 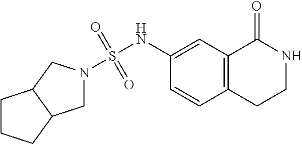 [SMILES: O=C1NCCc2ccc(NS(=O)(=O)N3CC4CCCC4C3)cc21] | 49 |
| 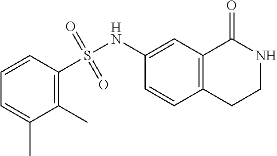 [SMILES: Cc1cccc(S(=O)(=O)Nc2ccc3c(c2)C(=O)NCC3)c1C] | 50 |
| 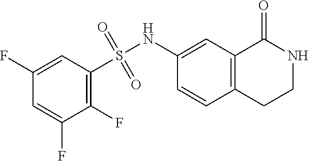 [SMILES: O=C1NCCc2ccc(NS(=O)(=O)c3cc(F)cc(F)c3F)cc21] | 51 |
| 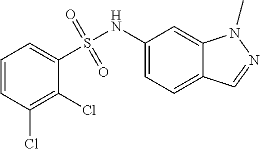 [SMILES: Cn1ncc2ccc(NS(=O)(=O)c3cccc(Cl)c3Cl)cc21] | 52 |
| 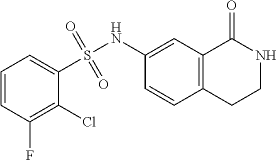 [SMILES: O=C1NCCc2ccc(NS(=O)(=O)c3cccc(F)c3Cl)cc21] | 53 |
| 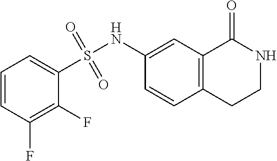 [SMILES: O=C1NCCc2ccc(NS(=O)(=O)c3cccc(F)c3F)cc21] | 54 |
| 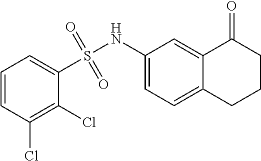 [SMILES: O=C1CCCc2ccc(NS(=O)(=O)c3cccc(Cl)c3Cl)cc21] | 55 |
| 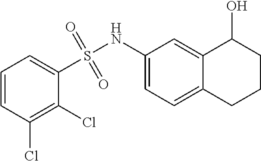 [SMILES: O=S(=O)(Nc1ccc2c(c1)C(O)CCC2)c1cccc(Cl)c1Cl] | 56 |
| 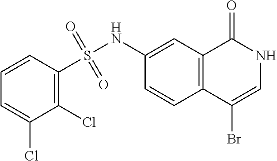 [SMILES: O=c1[nH]cc(Br)c2ccc(NS(=O)(=O)c3cccc(Cl)c3Cl)cc12] | 57 |
| 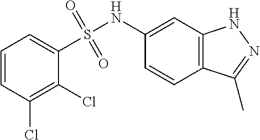 [SMILES: Cc1n[nH]c2cc(NS(=O)(=O)c3cccc(Cl)c3Cl)ccc12] | 58 |
| 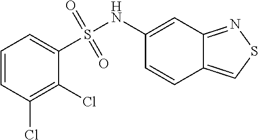 [SMILES: O=S(=O)(Nc1ccc2csnc2c1)c1cccc(Cl)c1Cl] | 59 |
| 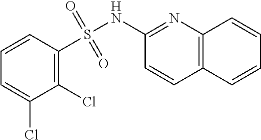 [SMILES: O=S(=O)(Nc1ccc2ccccc2n1)c1cccc(Cl)c1Cl] | 60 |
| 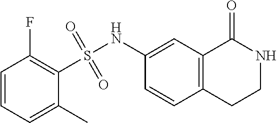 [SMILES: Cc1cccc(F)c1S(=O)(=O)Nc1ccc2c(c1)C(=O)NCC2] | 61 |
| 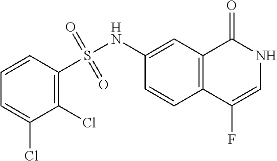 [SMILES: O=c1[nH]cc(F)c2ccc(NS(=O)(=O)c3cccc(Cl)c3Cl)cc12] | 62 |
| 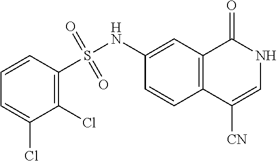 [SMILES: N#Cc1c[nH]c(=O)c2cc(NS(=O)(=O)c3cccc(Cl)c3Cl)ccc12] | 63 |
| 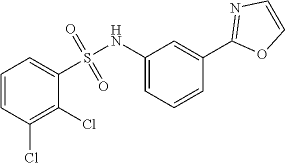 [SMILES: O=S(=O)(Nc1cccc(-c2ncco2)c1)c1cccc(Cl)c1Cl] | 64 |
| 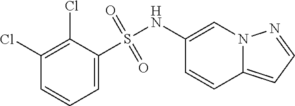 [SMILES: O=S(=O)(Nc1ccc2ccnn2c1)c1cccc(Cl)c1Cl] | 65 |
| 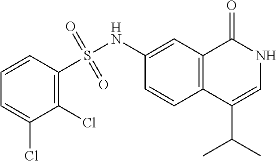 [SMILES: CC(C)c1c[nH]c(=O)c2cc(NS(=O)(=O)c3cccc(Cl)c3Cl)ccc12] | 66 |
| 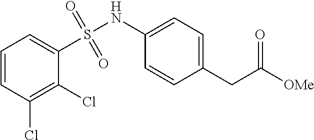 [SMILES: COC(=O)Cc1ccc(NS(=O)(=O)c2cccc(Cl)c2Cl)cc1] | 67 |
| 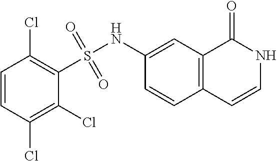 [SMILES: O=c1[nH]ccc2ccc(NS(=O)(=O)c3c(Cl)ccc(Cl)c3Cl)cc12] | 68 |
| 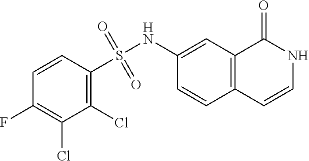 [SMILES: O=c1[nH]ccc2ccc(NS(=O)(=O)c3ccc(F)c(Cl)c3Cl)cc12] | 69 |
| 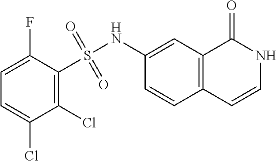 [SMILES: O=c1[nH]ccc2ccc(NS(=O)(=O)c3c(F)ccc(Cl)c3Cl)cc12] | 70 |
| 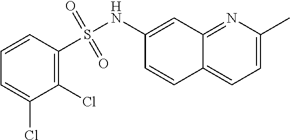 [SMILES: Cc1ccc2ccc(NS(=O)(=O)c3cccc(Cl)c3Cl)cc2n1] | 71 |
| 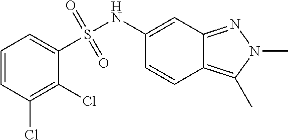 [SMILES: Cc1c2ccc(NS(=O)(=O)c3cccc(Cl)c3Cl)cc2nn1C] | 72 |
| 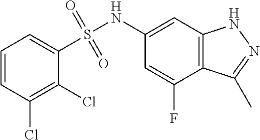 [SMILES: Cc1n[nH]c2cc(NS(=O)(=O)c3cccc(Cl)c3Cl)cc(F)c12] | 73 |
| 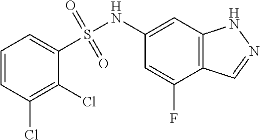 [SMILES: O=S(=O)(Nc1cc(F)c2cn[nH]c2c1)c1cccc(Cl)c1Cl] | 74 |
| 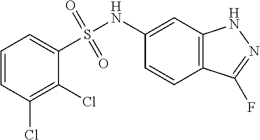 [SMILES: O=S(=O)(Nc1ccc2c(F)n[nH]c2c1)c1cccc(Cl)c1Cl] | 75 |
| 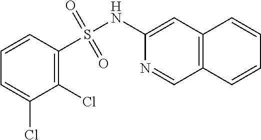 [SMILES: O=S(=O)(Nc1cc2ccccc2cn1)c1cccc(Cl)c1Cl] | 76 |
| 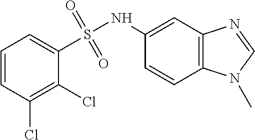 [SMILES: Cn1cnc2cc(NS(=O)(=O)c3cccc(Cl)c3Cl)ccc21] | 77 |
| 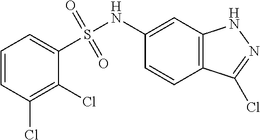 [SMILES: O=S(=O)(Nc1ccc2c(Cl)n[nH]c2c1)c1cccc(Cl)c1Cl] | 78 |
| 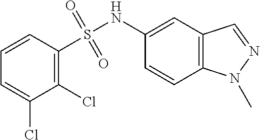 [SMILES: Cn1ncc2cc(NS(=O)(=O)c3cccc(Cl)c3Cl)ccc21] | 79 |
| 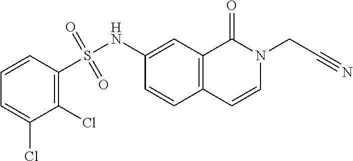 [SMILES: N#CCn1ccc2ccc(NS(=O)(=O)c3cccc(Cl)c3Cl)cc2c1=O] | 80 |
| 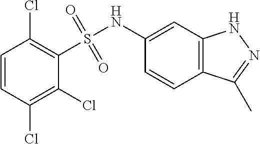 [SMILES: Cc1n[nH]c2cc(NS(=O)(=O)c3c(Cl)ccc(Cl)c3Cl)ccc12] | 81 |
| 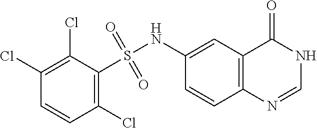 [SMILES: O=c1[nH]cnc2ccc(NS(=O)(=O)c3c(Cl)ccc(Cl)c3Cl)cc12] | 82 |
| 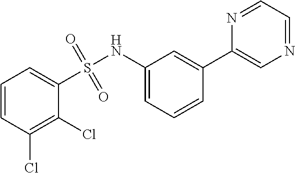 [SMILES: O=S(=O)(Nc1cccc(-c2cnccn2)c1)c1cccc(Cl)c1Cl] | 83 |
| 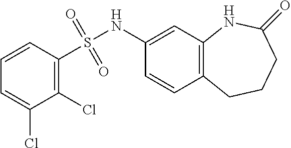 [SMILES: O=C1CCCc2ccc(NS(=O)(=O)c3cccc(Cl)c3Cl)cc2N1] | 84 |
| 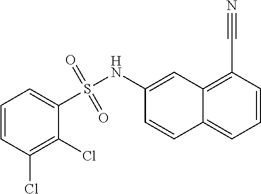 [SMILES: N#Cc1cccc2ccc(NS(=O)(=O)c3cccc(Cl)c3Cl)cc12] | 85 |
| 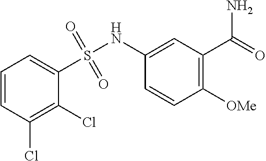 [SMILES: COc1ccc(NS(=O)(=O)c2cccc(Cl)c2Cl)cc1C(N)=O] | 86 |
| 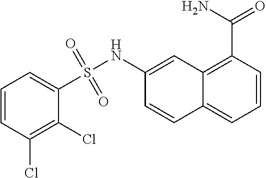 [SMILES: NC(=O)c1cccc2ccc(NS(=O)(=O)c3cccc(Cl)c3Cl)cc12] | 87 |
| 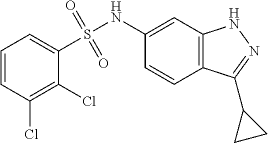 [SMILES: O=S(=O)(Nc1ccc2c(C3CC3)n[nH]c2c1)c1cccc(Cl)c1Cl] | 88 |
| 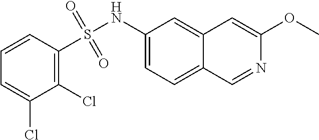 [SMILES: COc1cc2cc(NS(=O)(=O)c3cccc(Cl)c3Cl)ccc2cn1] | 89 |
| 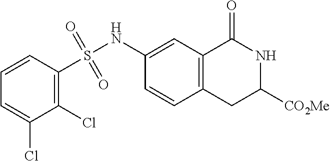 [SMILES: COC(=O)C1Cc2ccc(NS(=O)(=O)c3cccc(Cl)c3Cl)cc2C(=O)N1] | 90 |
| 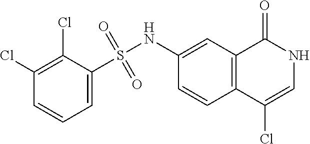 [SMILES: O=c1[nH]cc(Cl)c2ccc(NS(=O)(=O)c3cccc(Cl)c3Cl)cc12] | 91 |
| 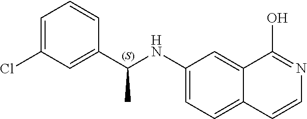 | 92 |
| 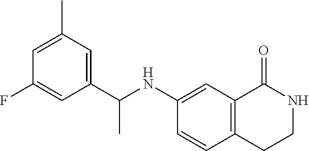 [SMILES: Cc1cc(F)cc(C(C)Nc2ccc3c(c2)C(=O)NCC3)c1] | 93 |
| 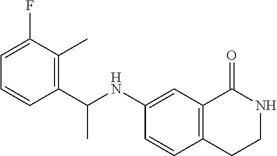 [SMILES: Cc1c(F)cccc1C(C)Nc1ccc2c(c1)C(=O)NCC2] | 94 |
| 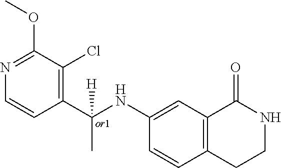 [SMILES: COc1nccc([C@H](C)Nc2ccc3c(c2)C(=O)NCC3)c1Cl] | 95 |
| 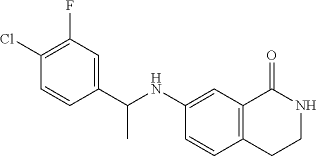 [SMILES: CC(Nc1ccc2c(c1)C(=O)NCC2)c1ccc(Cl)c(F)c1] | 96 |
| 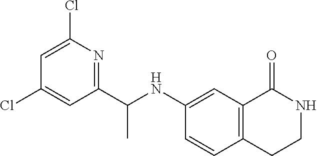 [SMILES: CC(Nc1ccc2c(c1)C(=O)NCC2)c1cc(Cl)cc(Cl)n1] | 97 |
| 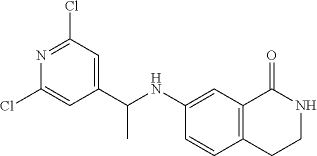 [SMILES: CC(Nc1ccc2c(c1)C(=O)NCC2)c1cc(Cl)nc(Cl)c1] | 98 |
| 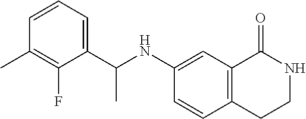 [SMILES: Cc1cccc(C(C)Nc2ccc3c(c2)C(=O)NCC3)c1F] | 99 |
| 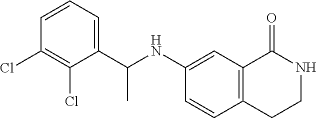 [SMILES: CC(Nc1ccc2c(c1)C(=O)NCC2)c1cccc(Cl)c1Cl] | 100 |
| 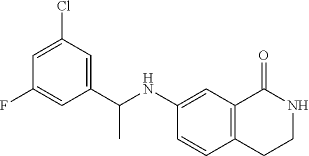 [SMILES: CC(Nc1ccc2c(c1)C(=O)NCC2)c1cc(F)cc(Cl)c1] | 101 |
| 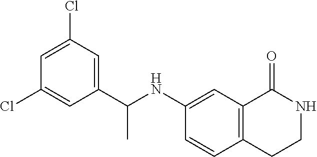 [SMILES: CC(Nc1ccc2c(c1)C(=O)NCC2)c1cc(Cl)cc(Cl)c1] | 102 |
| 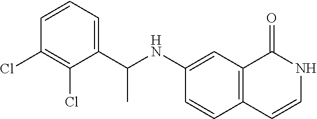 [SMILES: CC(Nc1ccc2cc[nH]c(=O)c2c1)c1cccc(Cl)c1Cl] | 103 |
| 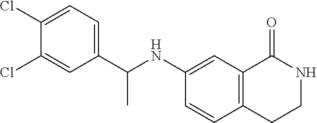 [SMILES: CC(Nc1ccc2c(c1)C(=O)NCC2)c1ccc(Cl)c(Cl)c1] | 104 |
| 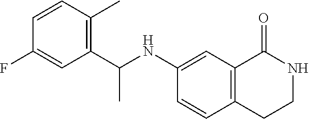 [SMILES: Cc1ccc(F)cc1C(C)Nc1ccc2c(c1)C(=O)NCC2] | 105 |
| 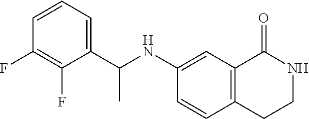 [SMILES: CC(Nc1ccc2c(c1)C(=O)NCC2)c1cccc(F)c1F] | 106 |
| 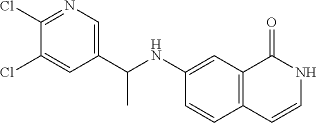 [SMILES: CC(Nc1ccc2cc[nH]c(=O)c2c1)c1cnc(Cl)c(Cl)c1] | 107 |
| 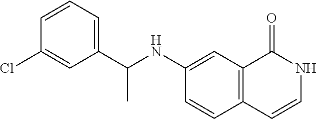 [SMILES: CC(Nc1ccc2cc[nH]c(=O)c2c1)c1cccc(Cl)c1] | 108 |
| 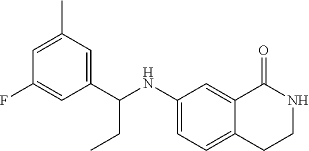 [SMILES: CCC(Nc1ccc2c(c1)C(=O)NCC2)c1cc(C)cc(F)c1] | 109 |
| 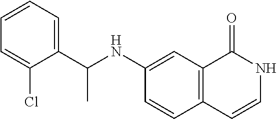 [SMILES: CC(Nc1ccc2cc[nH]c(=O)c2c1)c1ccccc1Cl] | 110 |
| 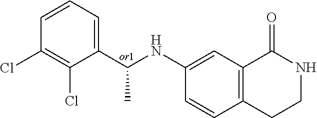 [SMILES: C[C@@H](Nc1ccc2c(c1)C(=O)NCC2)c1cccc(Cl)c1Cl] | 111 |
| 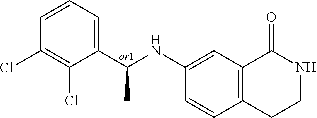 [SMILES: C[C@H](Nc1ccc2c(c1)C(=O)NCC2)c1cccc(Cl)c1Cl] | 112 |
| 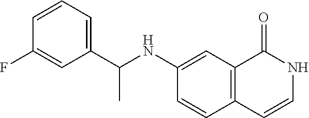 [SMILES: CC(Nc1ccc2cc[nH]c(=O)c2c1)c1cccc(F)c1] | 113 |
| 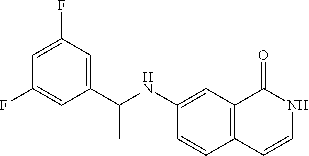 [SMILES: CC(Nc1ccc2cc[nH]c(=O)c2c1)c1cc(F)cc(F)c1] | 114 |
| 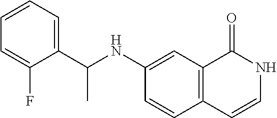 [SMILES: CC(Nc1ccc2cc[nH]c(=O)c2c1)c1ccccc1F] | 115 |
| 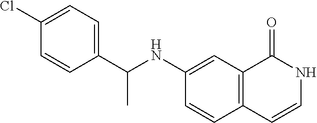 [SMILES: CC(Nc1ccc2cc[nH]c(=O)c2c1)c1ccc(Cl)cc1] | 116 |
| 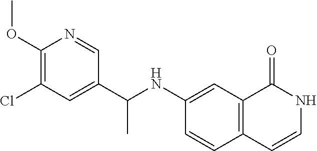 [SMILES: COc1ncc(C(C)Nc2ccc3cc[nH]c(=O)c3c2)cc1Cl] | 117 |
| 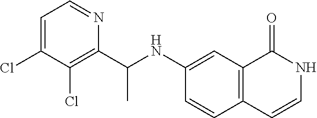 [SMILES: CC(Nc1ccc2cc[nH]c(=O)c2c1)c1nccc(Cl)c1Cl] | 118 |
| 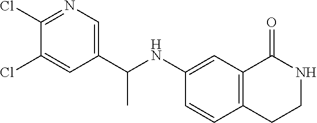 [SMILES: CC(Nc1ccc2c(c1)C(=O)NCC2)c1cnc(Cl)c(Cl)c1] | 119 |
| 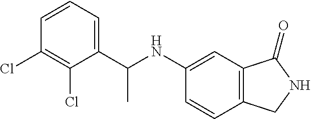 [SMILES: CC(Nc1ccc2c(c1)C(=O)NC2)c1cccc(Cl)c1Cl] | 120 |
| 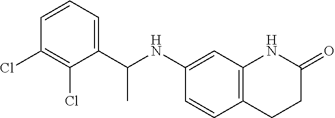 [SMILES: CC(Nc1ccc2c(c1)NC(=O)CC2)c1cccc(Cl)c1Cl] | 121 |
| 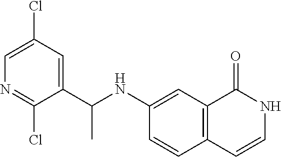 [SMILES: CC(Nc1ccc2cc[nH]c(=O)c2c1)c1cc(Cl)cnc1Cl] | 122 |
| 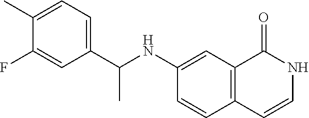 [SMILES: Cc1ccc(C(C)Nc2ccc3cc[nH]c(=O)c3c2)cc1F] | 123 |
| 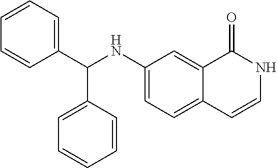 [SMILES: O=c1[nH]ccc2ccc(NC(c3ccccc3)c3ccccc3)cc12] | 124 |
| 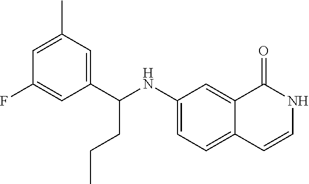 [SMILES: CCCC(Nc1ccc2cc[nH]c(=O)c2c1)c1cc(C)cc(F)c1] | 125 |
| 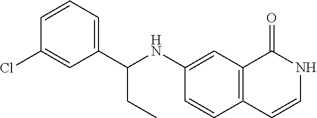 [SMILES: CCC(Nc1ccc2cc[nH]c(=O)c2c1)c1cccc(Cl)c1] | 126 |
| 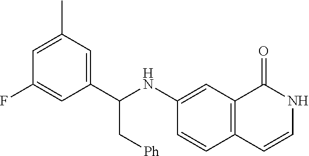 [SMILES: Cc1cc(F)cc(C(Cc2ccccc2)Nc2ccc3cc[nH]c(=O)c3c2)c1] | 127 |
| 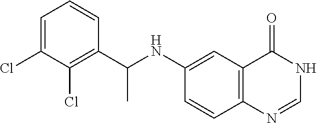 [SMILES: CC(Nc1ccc2nc[nH]c(=O)c2c1)c1cccc(Cl)c1Cl] | 128 |
| 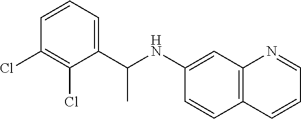 [SMILES: CC(Nc1ccc2cccnc2c1)c1cccc(Cl)c1Cl] | 129 |
| 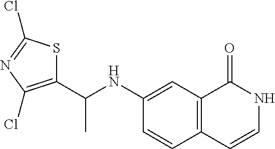 [SMILES: CC(Nc1ccc2cc[nH]c(=O)c2c1)c1sc(Cl)nc1Cl] | 130 |
| 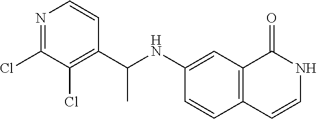 [SMILES: CC(Nc1ccc2cc[nH]c(=O)c2c1)c1ccnc(Cl)c1Cl] | 131 |
| 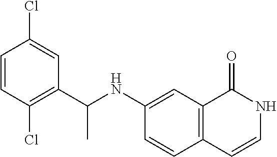 [SMILES: CC(Nc1ccc2cc[nH]c(=O)c2c1)c1cc(Cl)ccc1Cl] | 132 |
| 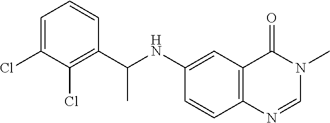 [SMILES: CC(Nc1ccc2ncn(C)c(=O)c2c1)c1cccc(Cl)c1Cl] | 133 |
| 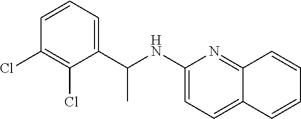 [SMILES: CC(Nc1ccc2ccccc2n1)c1cccc(Cl)c1Cl] | 134 |
| 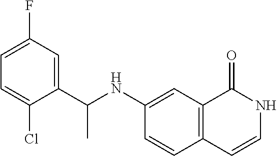 [SMILES: CC(Nc1ccc2cc[nH]c(=O)c2c1)c1cc(F)ccc1Cl] | 135 |
| 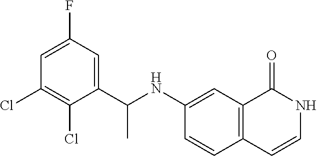 [SMILES: CC(Nc1ccc2cc[nH]c(=O)c2c1)c1cc(F)cc(Cl)c1Cl] | 136 |
| 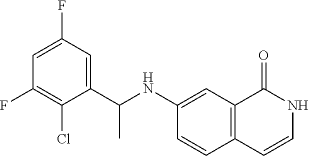 [SMILES: CC(Nc1ccc2cc[nH]c(=O)c2c1)c1cc(F)cc(F)c1Cl] | 137 |
| 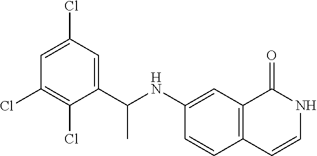 [SMILES: CC(Nc1ccc2cc[nH]c(=O)c2c1)c1cc(Cl)cc(Cl)c1Cl] | 138 |
| 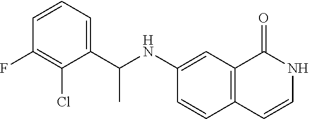 [SMILES: CC(Nc1ccc2cc[nH]c(=O)c2c1)c1cccc(F)c1Cl] | 139 |
| 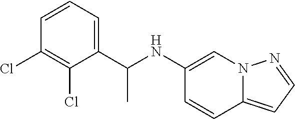 [SMILES: CC(Nc1ccc2ccnn2c1)c1cccc(Cl)c1Cl] | 140 |
| 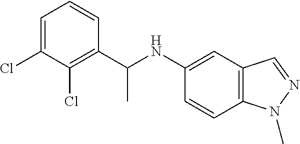 [SMILES: CC(Nc1ccc2c(cnn2C)c1)c1cccc(Cl)c1Cl] | 141 |
| 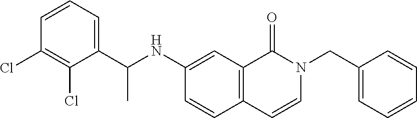 [SMILES: CC(Nc1ccc2ccn(Cc3ccccc3)c(=O)c2c1)c1cccc(Cl)c1Cl] | 142 |
| 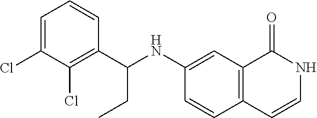 [SMILES: CCC(Nc1ccc2cc[nH]c(=O)c2c1)c1cccc(Cl)c1Cl] | 143 |
| 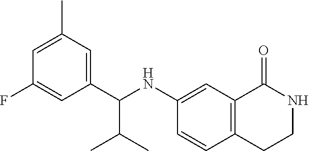 [SMILES: Cc1cc(F)cc(C(Nc2ccc3c(c2)C(=O)NCC3)C(C)C)c1] | 144 |
| 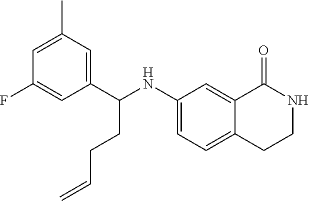 [SMILES: C=CCCC(Nc1ccc2c(c1)C(=O)NCC2)c1cc(C)cc(F)c1] | 145 |
| 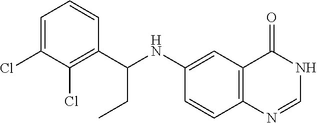 [SMILES: CCC(Nc1ccc2nc[nH]c(=O)c2c1)c1cccc(Cl)c1Cl] | 146 |
| 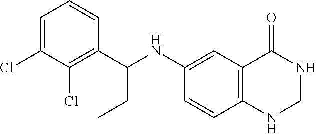 [SMILES: CCC(Nc1ccc2c(c1)C(=O)NCN2)c1cccc(Cl)c1Cl] | 147 |
| 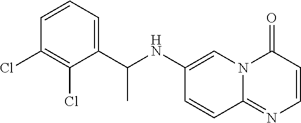 [SMILES: CC(Nc1ccc2nccc(=O)n2c1)c1cccc(Cl)c1Cl] | 148 |
| 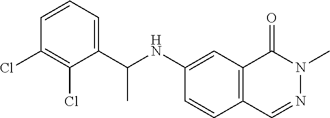 [SMILES: CC(Nc1ccc2cnn(C)c(=O)c2c1)c1cccc(Cl)c1Cl] | 149 |
| 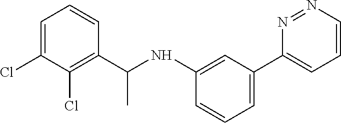 [SMILES: CC(Nc1cccc(-c2cccnn2)c1)c1cccc(Cl)c1Cl] | 150 |
| 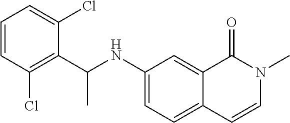 [SMILES: CC(Nc1ccc2ccn(C)c(=O)c2c1)c1c(Cl)cccc1Cl] | 151 |
| 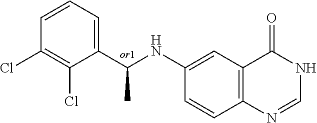 [SMILES: C[C@H](Nc1ccc2nc[nH]c(=O)c2c1)c1cccc(Cl)c1Cl] | 152 |
| 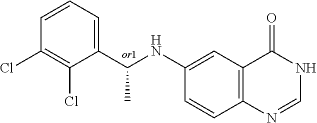 [SMILES: C[C@@H](Nc1ccc2nc[nH]c(=O)c2c1)c1cccc(Cl)c1Cl] | 152A |
| 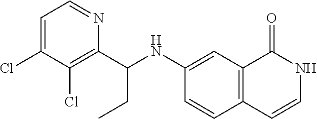 [SMILES: CCC(Nc1ccc2cc[nH]c(=O)c2c1)c1nccc(Cl)c1Cl] | 153 |
| 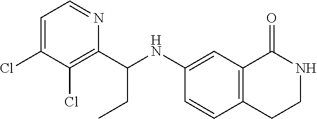 [SMILES: CCC(Nc1ccc2c(c1)C(=O)NCC2)c1nccc(Cl)c1Cl] | 154 |
| 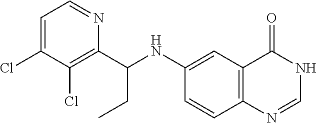 [SMILES: CCC(Nc1ccc2nc[nH]c(=O)c2c1)c1nccc(Cl)c1Cl] | 155 |
| 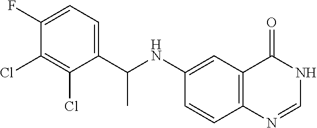 [SMILES: CC(Nc1ccc2nc[nH]c(=O)c2c1)c1ccc(F)c(Cl)c1Cl] | 156 |
| 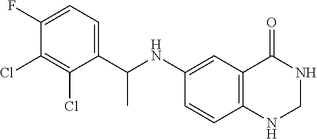 [SMILES: CC(Nc1ccc2c(c1)C(=O)NCN2)c1ccc(F)c(Cl)c1Cl] | 157 |
| 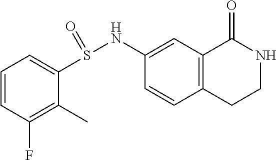 [SMILES: Cc1c(F)cccc1S(=O)Nc1ccc2c(c1)C(=O)NCC2] | 158 |
| 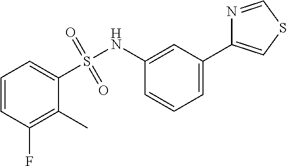 [SMILES: Cc1c(F)cccc1S(=O)(=O)Nc1cccc(-c2cscn2)c1] | 159 |
| 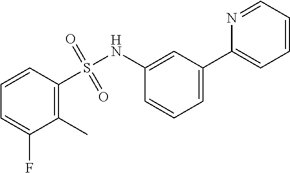 [SMILES: Cc1c(F)cccc1S(=O)(=O)Nc1cccc(-c2ccccn2)c1] | 160 |
| 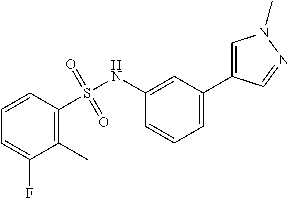 [SMILES: Cc1c(F)cccc1S(=O)(=O)Nc1cccc(-c2cnn(C)c2)c1] | 161 |
| 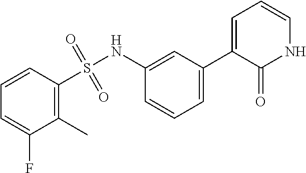 [SMILES: Cc1c(F)cccc1S(=O)(=O)Nc1cccc(-c2ccc[nH]c2=O)c1] | 162 |
| 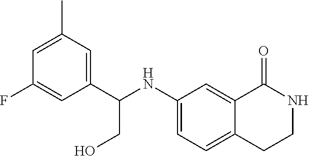 [SMILES: Cc1cc(F)cc(C(CO)Nc2ccc3c(c2)C(=O)NCC3)c1] | 163 |
| 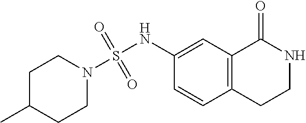 [SMILES: CC1CCN(S(=O)(=O)Nc2ccc3c(c2)C(=O)NCC3)CC1] | 164 |
| 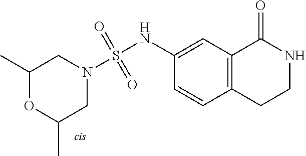 [SMILES: CC1CN(S(=O)(=O)Nc2ccc3c(c2)C(=O)NCC3)CC(C)O1] | 165 |
| 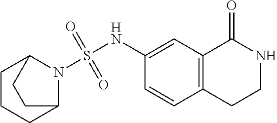 [SMILES: O=C1NCCc2ccc(NS(=O)(=O)N3C4CCCC3CC4)cc21] | 166 |
| 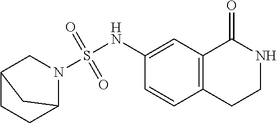 [SMILES: O=C1NCCc2ccc(NS(=O)(=O)N3CC4CCC3C4)cc21] | 167 |
| 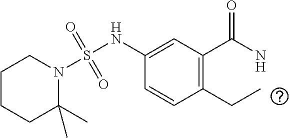 [SMILES: CCc1ccc(NS(=O)(=O)N2CCCCC2(C)C)cc1C(N)=O] | 168 |
| 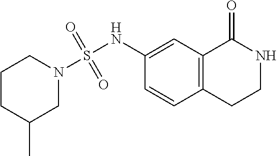 [SMILES: CC1CCCN(S(=O)(=O)Nc2ccc3c(c2)C(=O)NCC3)C1] | 169 |
| 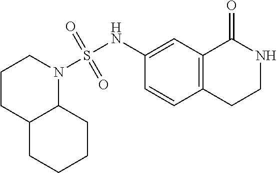 [SMILES: O=C1NCCc2ccc(NS(=O)(=O)N3CCCC4CCCCC43)cc21] | 170 |
| 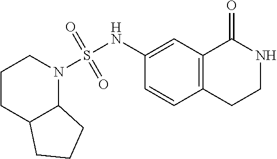 [SMILES: O=C1NCCc2ccc(NS(=O)(=O)N3CCCC4CCCC43)cc21] | 171 |
| 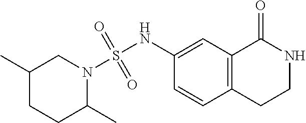 [SMILES: CC1CCC(C)N(S(=O)(=O)Nc2ccc3c(c2)C(=O)NCC3)C1] | 172 |
| 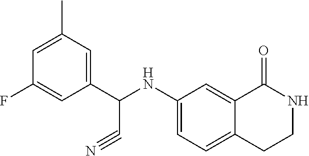 [SMILES: Cc1cc(F)cc(C(C#N)Nc2ccc3c(c2)C(=O)NCC3)c1] | 173 |
| 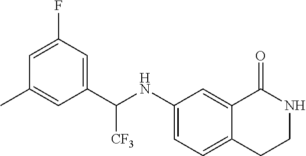 [SMILES: Cc1cc(F)cc(C(Nc2ccc3c(c2)C(=O)NCC3)C(F)(F)F)c1] | 174 |
| 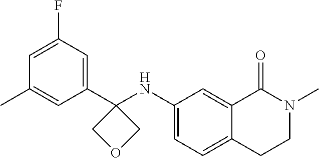 | 175 |
| 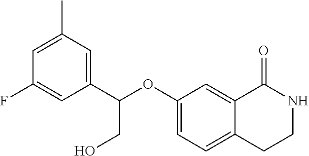 [SMILES: Cc1cc(F)cc(C(CO)Oc2ccc3c(c2)C(=O)NCC3)c1] | 176 |
| 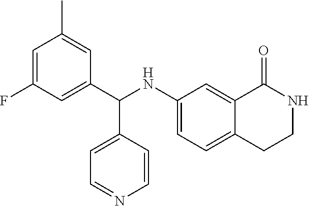 [SMILES: Cc1cc(F)cc(C(Nc2ccc3c(c2)C(=O)NCC3)c2ccncc2)c1] | 177 |
| 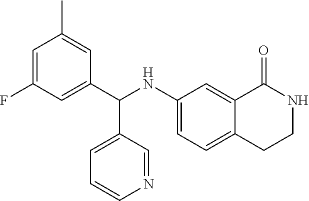 [SMILES: Cc1cc(F)cc(C(Nc2ccc3c(c2)C(=O)NCC3)c2cccnc2)c1] | 178 |
| 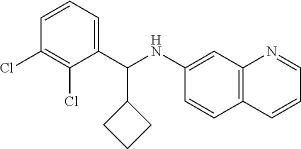 [SMILES: Clc1cccc(C(Nc2ccc3cccnc3c2)C2CCC2)c1Cl] | 179 |
| 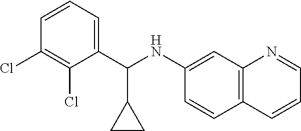 [SMILES: Clc1cccc(C(Nc2ccc3cccnc3c2)C2CC2)c1Cl] | 180 |
| 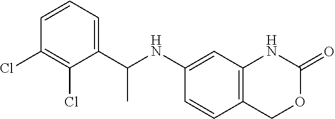 [SMILES: CC(Nc1ccc2c(c1)NC(=O)OC2)c1cccc(Cl)c1Cl] | 181 |
| 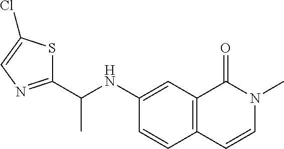 [SMILES: CC(Nc1ccc2ccn(C)c(=O)c2c1)c1ncc(Cl)s1] | 182 |
| 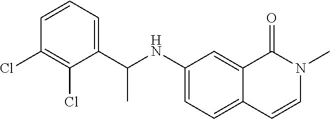 [SMILES: CC(Nc1ccc2ccn(C)c(=O)c2c1)c1cccc(Cl)c1Cl] | 183 |
| 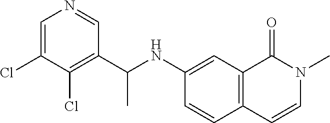 [SMILES: CC(Nc1ccc2ccn(C)c(=O)c2c1)c1cncc(Cl)c1Cl] | 184 |
| 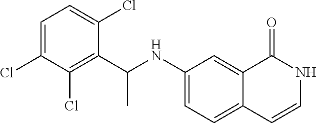 [SMILES: CC(Nc1ccc2cc[nH]c(=O)c2c1)c1c(Cl)ccc(Cl)c1Cl] | 185 |
| 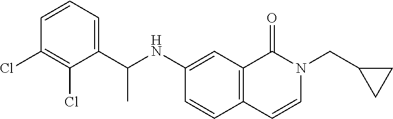 [SMILES: CC(Nc1ccc2ccn(CC3CC3)c(=O)c2c1)c1cccc(Cl)c1Cl] | 186 |
| 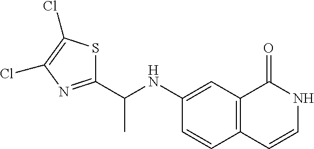 [SMILES: CC(Nc1ccc2cc[nH]c(=O)c2c1)c1nc(Cl)c(Cl)s1] | 187 |
| 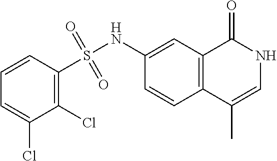 [SMILES: Cc1c[nH]c(=O)c2cc(NS(=O)(=O)c3cccc(Cl)c3Cl)ccc12] | 188 |
| 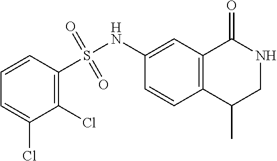 [SMILES: CC1CNC(=O)c2cc(NS(=O)(=O)c3cccc(Cl)c3Cl)ccc21] | 189 |
| 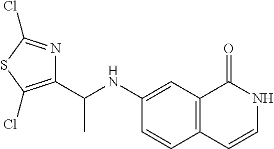 [SMILES: CC(Nc1ccc2cc[nH]c(=O)c2c1)c1nc(Cl)sc1Cl] | 190 |
| 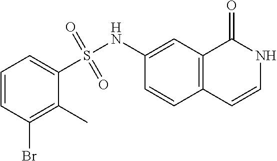 [SMILES: Cc1c(Br)cccc1S(=O)(=O)Nc1ccc2cc[nH]c(=O)c2c1] | 191 |
| 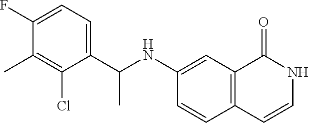 [SMILES: Cc1c(F)ccc(C(C)Nc2ccc3cc[nH]c(=O)c3c2)c1Cl] | 192 |
| 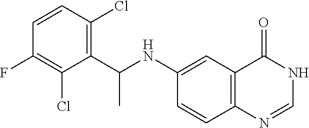 [SMILES: CC(Nc1ccc2nc[nH]c(=O)c2c1)c1c(Cl)ccc(F)c1Cl] | 193 |
| 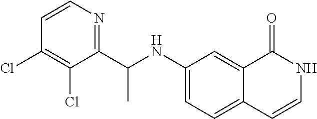 [SMILES: CC(Nc1ccc2cc[nH]c(=O)c2c1)c1nccc(Cl)c1Cl] | 194 |
| 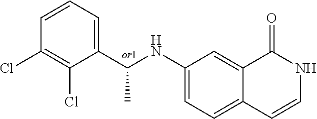 [SMILES: C[C@@H](Nc1ccc2cc[nH]c(=O)c2c1)c1cccc(Cl)c1Cl] | 194A |
| 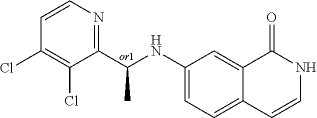 [SMILES: C[C@H](Nc1ccc2cc[nH]c(=O)c2c1)c1nccc(Cl)c1Cl] | 194B |
| 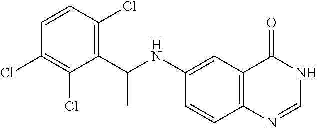 [SMILES: CC(Nc1ccc2nc[nH]c(=O)c2c1)c1c(Cl)ccc(Cl)c1Cl] | 195 |
| 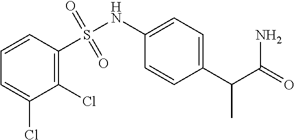 [SMILES: CC(C(N)=O)c1ccc(NS(=O)(=O)c2cccc(Cl)c2Cl)cc1] | 196 |
| 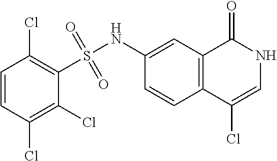 [SMILES: O=c1[nH]cc(Cl)c2ccc(NS(=O)(=O)c3c(Cl)ccc(Cl)c3Cl)cc12] | 197 |
| 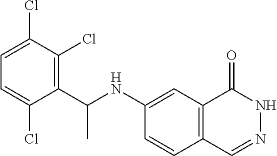 [SMILES: CC(Nc1ccc2cn[nH]c(=O)c2c1)c1c(Cl)ccc(Cl)c1Cl] | 198 |
| 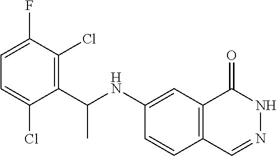 [SMILES: CC(Nc1ccc2cn[nH]c(=O)c2c1)c1c(Cl)ccc(F)c1Cl] | 199 |
| 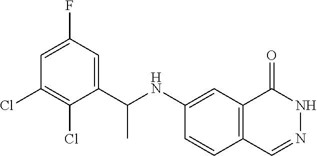 [SMILES: CC(Nc1ccc2cn[nH]c(=O)c2c1)c1cc(F)cc(Cl)c1Cl] | 200 |
| 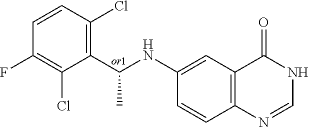 [SMILES: C[C@@H](Nc1ccc2nc[nH]c(=O)c2c1)c1c(Cl)ccc(F)c1Cl] | 201 |
| 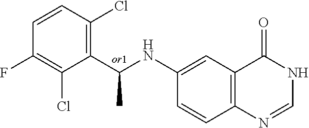 [SMILES: C[C@H](Nc1ccc2nc[nH]c(=O)c2c1)c1c(Cl)ccc(F)c1Cl] | 201A |
| 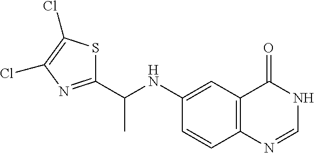 [SMILES: CC(Nc1ccc2nc[nH]c(=O)c2c1)c1nc(Cl)c(Cl)s1] | 202 |
| 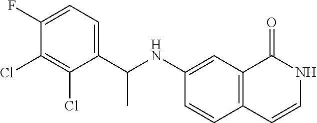 [SMILES: CC(Nc1ccc2cc[nH]c(=O)c2c1)c1ccc(F)c(Cl)c1Cl] | 203 |
| 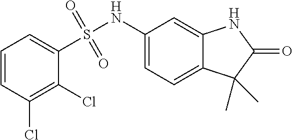 [SMILES: CC1(C)C(=O)Nc2cc(NS(=O)(=O)c3cccc(Cl)c3Cl)ccc21] | 204 |
| 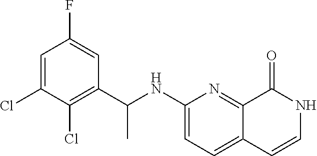 [SMILES: CC(Nc1ccc2cc[nH]c(=O)c2n1)c1cc(F)cc(Cl)c1Cl] | 205 |
| 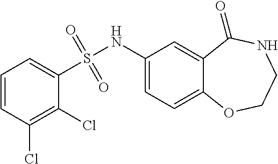 [SMILES: O=C1NCCOc2ccc(NS(=O)(=O)c3cccc(Cl)c3Cl)cc21] | 206 |
| 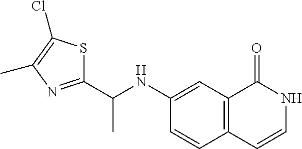 [SMILES: Cc1nc(C(C)Nc2ccc3cc[nH]c(=O)c3c2)sc1Cl] | 207 |
| 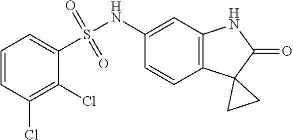 [SMILES: O=C1Nc2cc(NS(=O)(=O)c3cccc(Cl)c3Cl)ccc2C12CC2] | 208 |
| 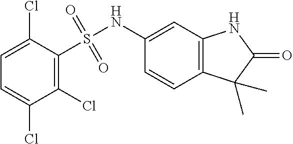 [SMILES: CC1(C)C(=O)Nc2cc(NS(=O)(=O)c3c(Cl)ccc(Cl)c3Cl)ccc21] | 209 |
| 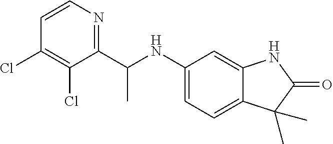 [SMILES: CC(Nc1ccc2c(c1)NC(=O)C2(C)C)c1nccc(Cl)c1Cl] | 210 |
| 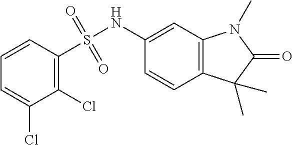 [SMILES: CN1C(=O)C(C)(C)c2ccc(NS(=O)(=O)c3cccc(Cl)c3Cl)cc21] | 211 |
| 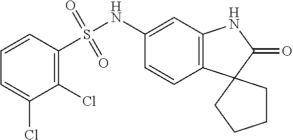 [SMILES: O=C1Nc2cc(NS(=O)(=O)c3cccc(Cl)c3Cl)ccc2C12CCCC2] | 212 |
| 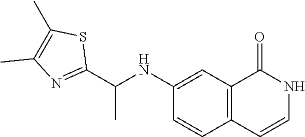 [SMILES: Cc1nc(C(C)Nc2ccc3cc[nH]c(=O)c3c2)sc1C] | 213 |
| 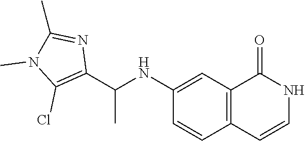 [SMILES: Cc1nc(C(C)Nc2ccc3cc[nH]c(=O)c3c2)c(Cl)n1C] | 214 |
| 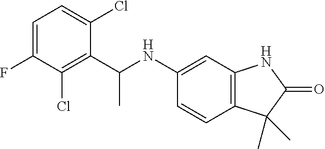 [SMILES: CC(Nc1ccc2c(c1)NC(=O)C2(C)C)c1c(Cl)ccc(F)c1Cl] | 215 |
| 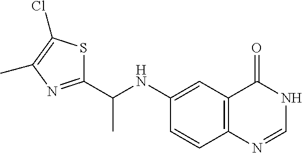 [SMILES: Cc1nc(C(C)Nc2ccc3nc[nH]c(=O)c3c2)sc1Cl] | 216 |
| 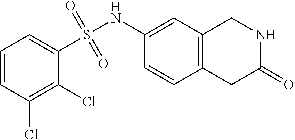 [SMILES: O=C1Cc2ccc(NS(=O)(=O)c3cccc(Cl)c3Cl)cc2CN1] | 217 |
| 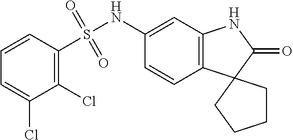 [SMILES: O=C1Nc2cc(NS(=O)(=O)c3cccc(Cl)c3Cl)ccc2C12CCCC2] | 218 |
| 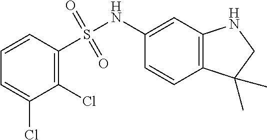 [SMILES: CC1(C)CNc2cc(NS(=O)(=O)c3cccc(Cl)c3Cl)ccc21] | 219 |
| 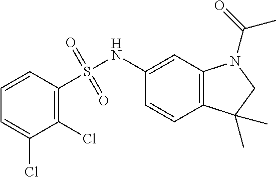 [SMILES: CC(=O)N1CC(C)(C)c2ccc(NS(=O)(=O)c3cccc(Cl)c3Cl)cc21] | 220 |
| 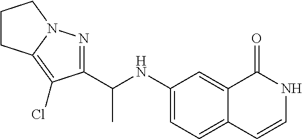 [SMILES: CC(Nc1ccc2cc[nH]c(=O)c2c1)c1nn2c(c1Cl)CCC2] | 221 |
| 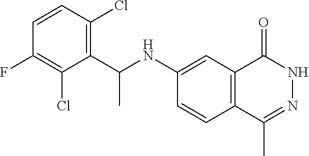 [SMILES: Cc1n[nH]c(=O)c2cc(NC(C)c3c(Cl)ccc(F)c3Cl)ccc12] | 222 |
| 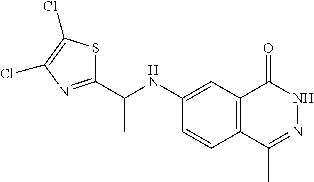 [SMILES: Cc1n[nH]c(=O)c2cc(NC(C)c3nc(Cl)c(Cl)s3)ccc12] | 223 |
| 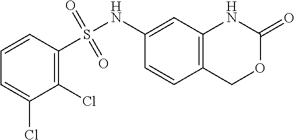 [SMILES: O=C1Nc2cc(NS(=O)(=O)c3cccc(Cl)c3Cl)ccc2CO1] | 224 |
| 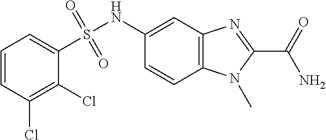 [SMILES: Cn1c(C(N)=O)nc2cc(NS(=O)(=O)c3cccc(Cl)c3Cl)ccc21] | 225 |
| 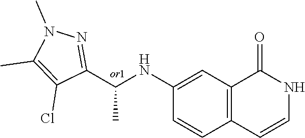 [SMILES: Cc1c(Cl)c([C@@H](C)Nc2ccc3cc[nH]c(=O)c3c2)nn1C] | 226A |
| 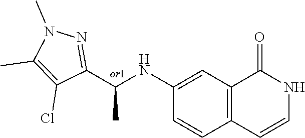 [SMILES: Cc1c(Cl)c([C@H](C)Nc2ccc3cc[nH]c(=O)c3c2)nn1C] | 226B |
| 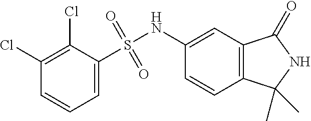 [SMILES: CC1(C)NC(=O)c2cc(NS(=O)(=O)c3cccc(Cl)c3Cl)ccc21] | 227 |
| 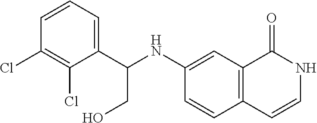 [SMILES: O=c1[nH]ccc2ccc(NC(CO)c3cccc(Cl)c3Cl)cc12] | 228 |
| 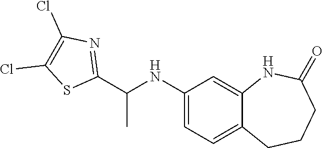 [SMILES: CC(Nc1ccc2c(c1)NC(=O)CCC2)c1nc(Cl)c(Cl)s1] | 229 |
| 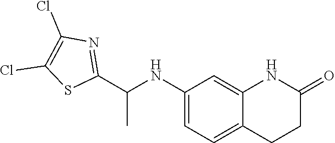 [SMILES: CC(Nc1ccc2c(c1)NC(=O)CC2)c1nc(Cl)c(Cl)s1] | 230 |
| 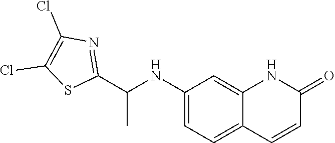 [SMILES: CC(Nc1ccc2ccc(=O)[nH]c2c1)c1nc(Cl)c(Cl)s1] | 231 |
| 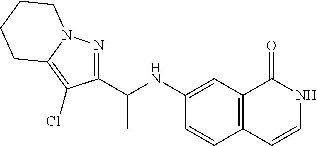 [SMILES: CC(Nc1ccc2cc[nH]c(=O)c2c1)c1nn2c(c1Cl)CCCC2] | 232 |
| 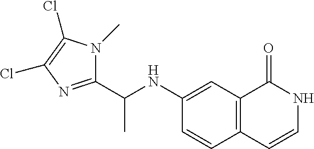 [SMILES: CC(Nc1ccc2cc[nH]c(=O)c2c1)c1nc(Cl)c(Cl)n1C] | 233 |
| 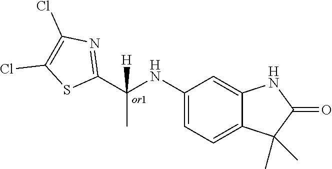 [SMILES: C[C@@H](Nc1ccc2c(c1)NC(=O)C2(C)C)c1nc(Cl)c(Cl)s1] | 234 |
| 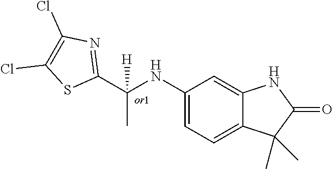 [SMILES: C[C@H](Nc1ccc2c(c1)NC(=O)C2(C)C)c1nc(Cl)c(Cl)s1] | 234A |
| 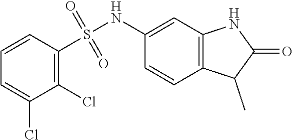 [SMILES: CC1C(=O)Nc2cc(NS(=O)(=O)c3cccc(Cl)c3Cl)ccc21] | 235 |
| 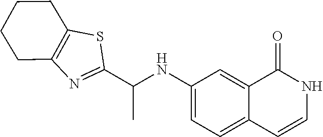 [SMILES: CC(Nc1ccc2cc[nH]c(=O)c2c1)c1nc2c(s1)CCCC2] | 236 |
| 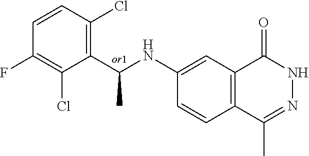 [SMILES: Cc1n[nH]c(=O)c2cc(N[C@@H](C)c3c(Cl)ccc(F)c3Cl)ccc12] | 237 |
| 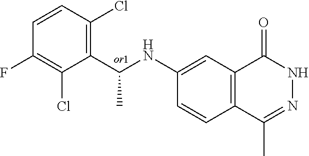 [SMILES: Cc1n[nH]c(=O)c2cc(N[C@H](C)c3c(Cl)ccc(F)c3Cl)ccc12] | 237A |
| 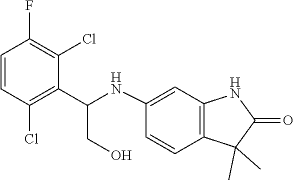 [SMILES: CC1(C)C(=O)Nc2cc(NC(CO)c3c(Cl)ccc(F)c3Cl)ccc21] | 238 |
| 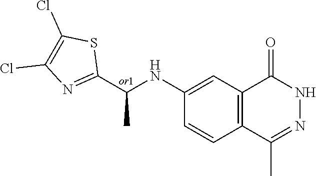 [SMILES: Cc1n[nH]c(=O)c2cc(N[C@@H](C)c3nc(Cl)c(Cl)s3)ccc12] | 239A |
| 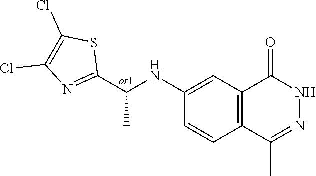 [SMILES: Cc1n[nH]c(=O)c2cc(N[C@H](C)c3nc(Cl)c(Cl)s3)ccc12] | 239B |
| 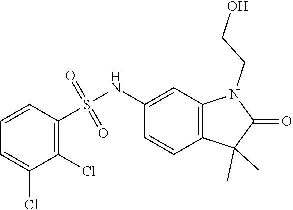 [SMILES: CC1(C)C(=O)N(CCO)c2cc(NS(=O)(=O)c3cccc(Cl)c3Cl)ccc21] | 240 |
| 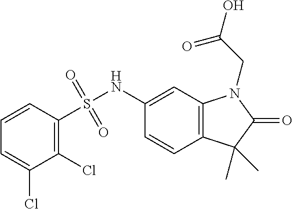 [SMILES: CC1(C)C(=O)N(CC(=O)O)c2cc(NS(=O)(=O)c3cccc(Cl)c3Cl)ccc21] | 241 |
| 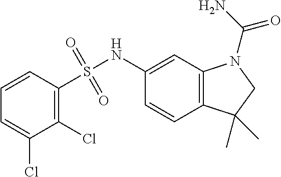 [SMILES: CC1(C)CN(C(N)=O)c2cc(NS(=O)(=O)c3cccc(Cl)c3Cl)ccc21] | 242 |
| 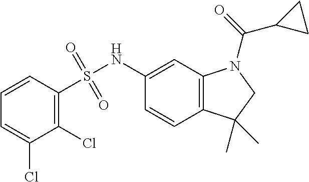 [SMILES: CC1(C)CN(C(=O)C2CC2)c2cc(NS(=O)(=O)c3cccc(Cl)c3Cl)ccc21] | 243 |
| 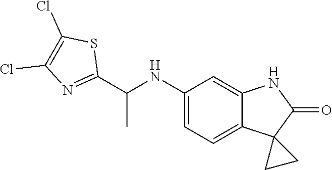 [SMILES: CC(Nc1ccc2c(c1)NC(=O)C21CC1)c1nc(Cl)c(Cl)s1] | 244 |
| 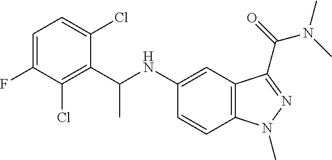 [SMILES: CC(Nc1ccc2c(c1)c(C(=O)N(C)C)nn2C)c1c(Cl)ccc(F)c1Cl] | 245 |
| 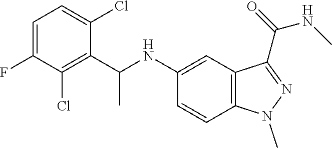 [SMILES: CNC(=O)c1nn(C)c2ccc(NC(C)c3c(Cl)ccc(F)c3Cl)cc12] | 246 |
| 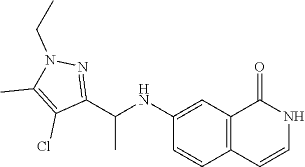 [SMILES: CCn1nc(C(C)Nc2ccc3cc[nH]c(=O)c3c2)c(Cl)c1C] | 247 |
| 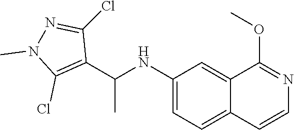 [SMILES: COc1nccc2ccc(NC(C)c3c(Cl)nn(C)c3Cl)cc12] | 248 |
| 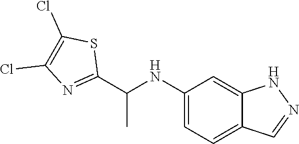 [SMILES: CC(Nc1ccc2cn[nH]c2c1)c1nc(Cl)c(Cl)s1] | 249 |
| 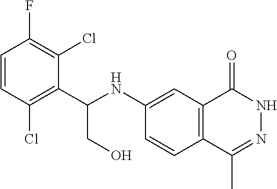 [SMILES: Cc1n[nH]c(=O)c2cc(NC(CO)c3c(Cl)ccc(F)c3Cl)ccc12] | 250 |
| 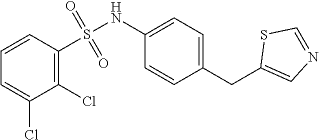 [SMILES: O=S(=O)(Nc1ccc(Cc2cncs2)cc1)c1cccc(Cl)c1Cl] | 251 |
| 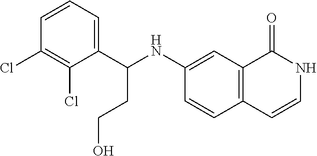 [SMILES: O=c1[nH]ccc2ccc(NC(CCO)c3cccc(Cl)c3Cl)cc12] | 252 |
| 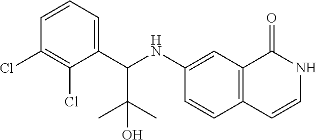 [SMILES: CC(C)(O)C(Nc1ccc2cc[nH]c(=O)c2c1)c1cccc(Cl)c1Cl] | 253 |
| 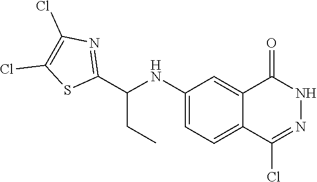 [SMILES: CCC(Nc1ccc2c(Cl)n[nH]c(=O)c2c1)c1nc(Cl)c(Cl)s1] | 254 |
| 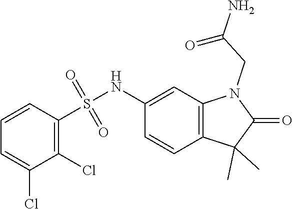 [SMILES: CC1(C)C(=O)N(CC(N)=O)c2cc(NS(=O)(=O)c3cccc(Cl)c3Cl)ccc21] | 255 |
| 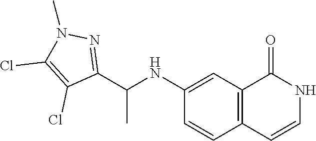 [SMILES: CC(Nc1ccc2cc[nH]c(=O)c2c1)c1nn(C)c(Cl)c1Cl] | 256 |
| 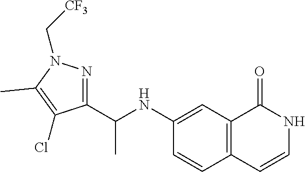 [SMILES: Cc1c(Cl)c(C(C)Nc2ccc3cc[nH]c(=O)c3c2)nn1CC(F)(F)F] | 257 |
| 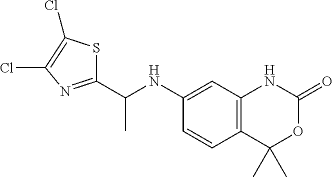 [SMILES: CC(Nc1ccc2c(c1)NC(=O)OC2(C)C)c1nc(Cl)c(Cl)s1] | 258 |
| 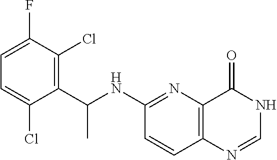 [SMILES: CC(Nc1ccc2nc[nH]c(=O)c2n1)c1c(Cl)ccc(F)c1Cl] | 259 |
| 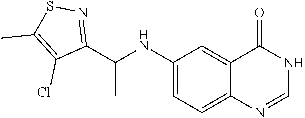 [SMILES: Cc1snc(C(C)Nc2ccc3nc[nH]c(=O)c3c2)c1Cl] | 260 |
| 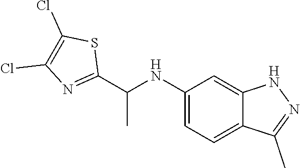 [SMILES: Cc1n[nH]c2cc(NC(C)c3nc(Cl)c(Cl)s3)ccc12] | 261 |
| 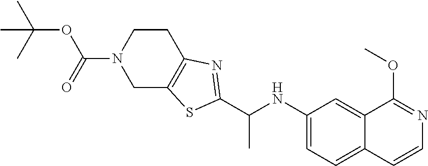 [SMILES: COc1nccc2ccc(NC(C)c3nc4c(s3)CN(C(=O)OC(C)(C)C)CC4)cc12] | 262 |
| 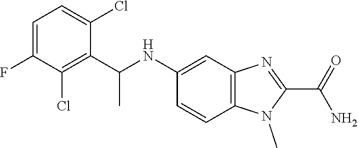 [SMILES: CC(Nc1ccc2c(c1)nc(C(N)=O)n2C)c1c(Cl)ccc(F)c1Cl] | 263 |
| 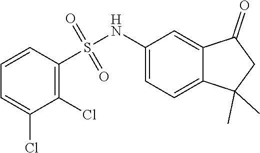 [SMILES: CC1(C)CC(=O)c2cc(NS(=O)(=O)c3cccc(Cl)c3Cl)ccc21] | 264 |
| 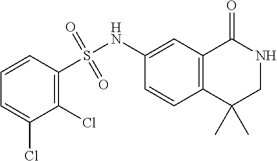 [SMILES: CC1(C)CNC(=O)c2cc(NS(=O)(=O)c3cccc(Cl)c3Cl)ccc21] | 265 |
| 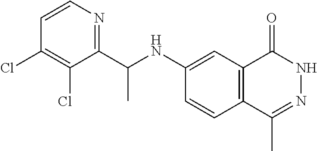 [SMILES: Cc1n[nH]c(=O)c2cc(NC(C)c3nccc(Cl)c3Cl)ccc12] | 266 |
| 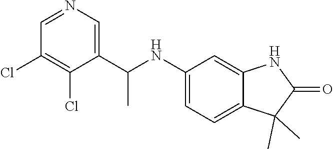 [SMILES: CC(Nc1ccc2c(c1)NC(=O)C2(C)C)c1cncc(Cl)c1Cl] | 267 |
| 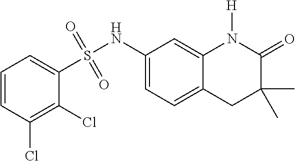 [SMILES: CC1(C)Cc2ccc(NS(=O)(=O)c3cccc(Cl)c3Cl)cc2NC1=O] | 268 |
| 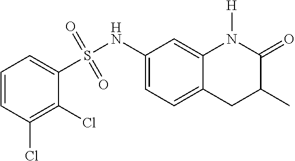 [SMILES: CC1Cc2ccc(NS(=O)(=O)c3cccc(Cl)c3Cl)cc2NC1=O] | 269 |
| 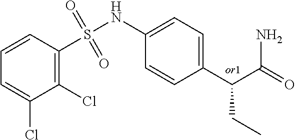 [SMILES: CC[C@@H](C(N)=O)c1ccc(NS(=O)(=O)c2cccc(Cl)c2Cl)cc1] | 270 |
| 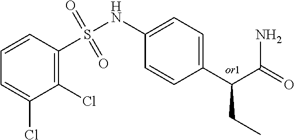 [SMILES: CC[C@H](C(N)=O)c1ccc(NS(=O)(=O)c2cccc(Cl)c2Cl)cc1] | 270A |
| 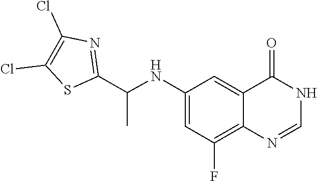 [SMILES: CC(Nc1cc(F)c2nc[nH]c(=O)c2c1)c1nc(Cl)c(Cl)s1] | 271 |
| 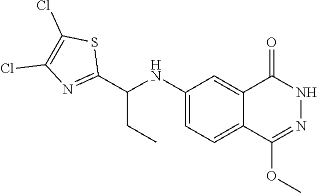 [SMILES: CCC(Nc1ccc2c(OC)n[nH]c(=O)c2c1)c1nc(Cl)c(Cl)s1] | 272 |
| 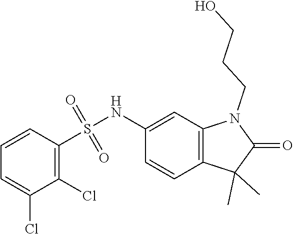 [SMILES: CC1(C)C(=O)N(CCCO)c2cc(NS(=O)(=O)c3cccc(Cl)c3Cl)ccc21] | 273 |
| 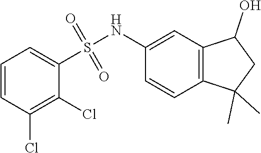 [SMILES: CC1(C)CC(O)c2cc(NS(=O)(=O)c3cccc(Cl)c3Cl)ccc21] | 274 |
| 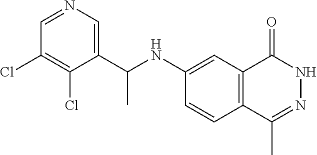 [SMILES: Cc1n[nH]c(=O)c2cc(NC(C)c3cncc(Cl)c3Cl)ccc12] | 275 |
| 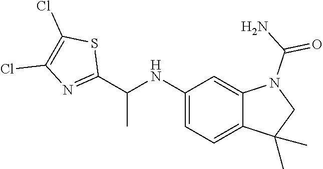 [SMILES: CC(Nc1ccc2c(c1)N(C(N)=O)CC2(C)C)c1nc(Cl)c(Cl)s1] | 276 |
| 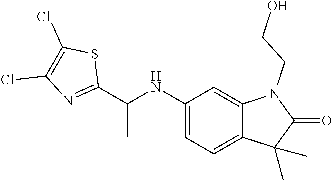 [SMILES: CC(Nc1ccc2c(c1)N(CCO)C(=O)C2(C)C)c1nc(Cl)c(Cl)s1] | 277 |
| 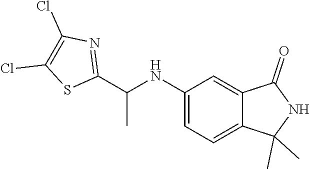 [SMILES: CC(Nc1ccc2c(c1)C(=O)NC2(C)C)c1nc(Cl)c(Cl)s1] | 278 |
| 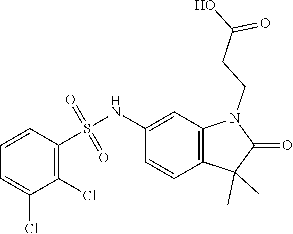 [SMILES: CC1(C)C(=O)N(CCC(=O)O)c2cc(NS(=O)(=O)c3cccc(Cl)c3Cl)ccc21] | 279 |
| 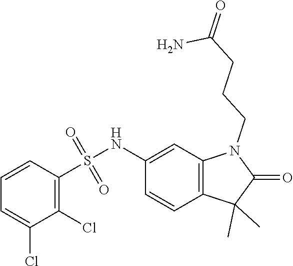 [SMILES: CC1(C)C(=O)N(CCCC(N)=O)c2cc(NS(=O)(=O)c3cccc(Cl)c3Cl)ccc21] | 280 |
| 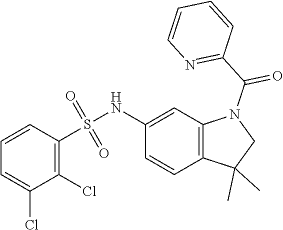 [SMILES: CC1(C)CN(C(=O)c2ccccn2)c2cc(NS(=O)(=O)c3cccc(Cl)c3Cl)ccc21] | 281 |
| 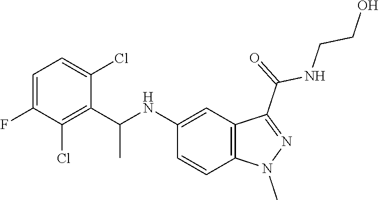 [SMILES: CC(Nc1ccc2c(c1)c(C(=O)NCCO)nn2C)c1c(Cl)ccc(F)c1Cl] | 282 |
| 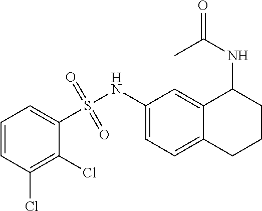 [SMILES: CC(=O)NC1CCCc2ccc(NS(=O)(=O)c3cccc(Cl)c3Cl)cc21] | 283 |
| 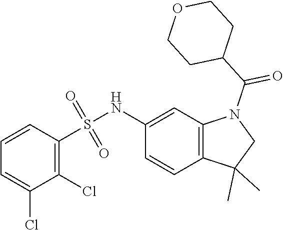 [SMILES: CC1(C)CN(C(=O)C2CCOCC2)c2cc(NS(=O)(=O)c3cccc(Cl)c3Cl)ccc21] | 284 |
| 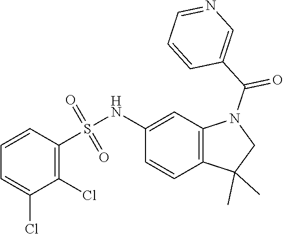 [SMILES: CC1(C)CN(C(=O)c2cccnc2)c2cc(NS(=O)(=O)c3cccc(Cl)c3Cl)ccc21] | 285 |
| 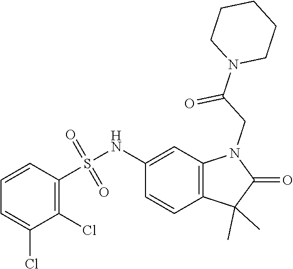 [SMILES: CC1(C)C(=O)N(CC(=O)N2CCCCC2)c2cc(NS(=O)(=O)c3cccc(Cl)c3Cl)ccc21] | 286 |
| 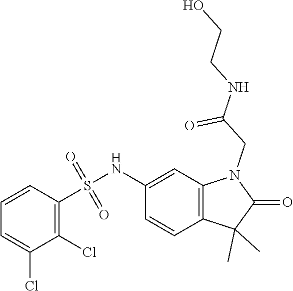 [SMILES: CC1(C)C(=O)N(CC(=O)NCCO)c2cc(NS(=O)(=O)c3cccc(Cl)c3Cl)ccc21] | 287 |
| 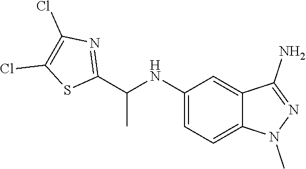 [SMILES: CC(Nc1ccc2c(c1)c(N)nn2C)c1nc(Cl)c(Cl)s1] | 288 |
| 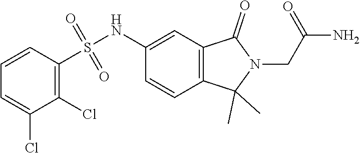 [SMILES: CC1(C)c2ccc(NS(=O)(=O)c3cccc(Cl)c3Cl)cc2C(=O)N1CC(N)=O] | 289 |
| 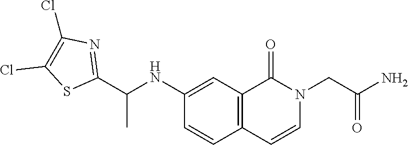 [SMILES: CC(Nc1ccc2ccn(CC(N)=O)c(=O)c2c1)c1nc(Cl)c(Cl)s1] | 290 |
| 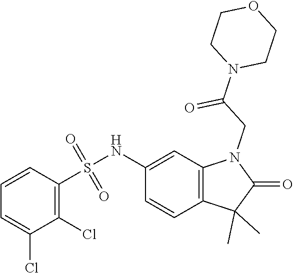 [SMILES: CC1(C)C(=O)N(CC(=O)N2CCOCC2)c2cc(NS(=O)(=O)c3cccc(Cl)c3Cl)ccc21] | 291 |
| 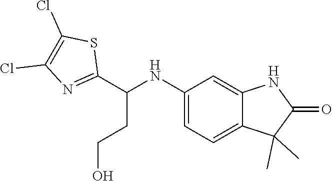 [SMILES: CC1(C)C(=O)Nc2cc(NC(CCO)c3nc(Cl)c(Cl)s3)ccc21] | 292 |
| 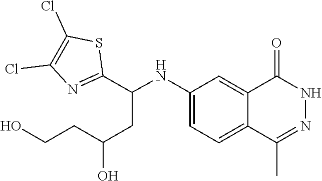 [SMILES: Cc1n[nH]c(=O)c2cc(NC(CC(O)CCO)c3nc(Cl)c(Cl)s3)ccc12] | 293 |
| 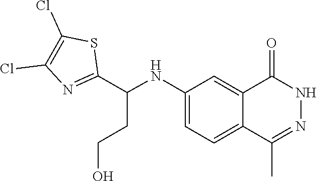 [SMILES: Cc1n[nH]c(=O)c2cc(NC(CCO)c3nc(Cl)c(Cl)s3)ccc12] | 294 |
| 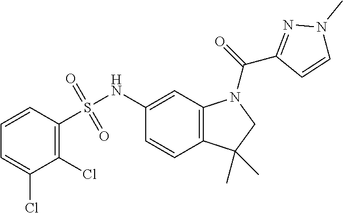 [SMILES: Cn1ccc(C(=O)N2CC(C)(C)c3ccc(NS(=O)(=O)c4cccc(Cl)c4Cl)cc32)n1] | 295 |
| 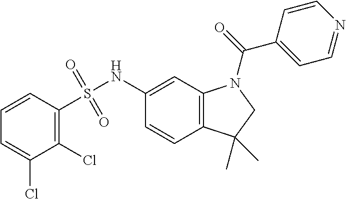 [SMILES: CC1(C)CN(C(=O)c2ccncc2)c2cc(NS(=O)(=O)c3cccc(Cl)c3Cl)ccc21] | 296 |
| 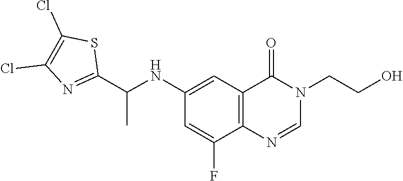 [SMILES: CC(Nc1cc(F)c2ncn(CCO)c(=O)c2c1)c1nc(Cl)c(Cl)s1] | 297 |
| 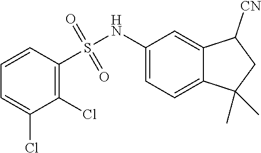 [SMILES: CC1(C)CC(C#N)c2cc(NS(=O)(=O)c3cccc(Cl)c3Cl)ccc21] | 298 |
| 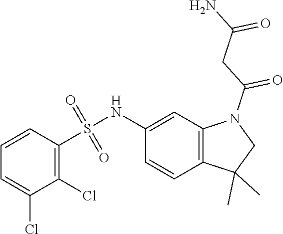 [SMILES: CC1(C)CN(C(=O)CC(N)=O)c2cc(NS(=O)(=O)c3cccc(Cl)c3Cl)ccc21] | 299 |
| 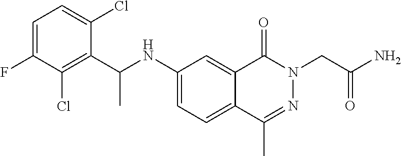 [SMILES: Cc1nn(CC(N)=O)c(=O)c2cc(NC(C)c3c(Cl)ccc(F)c3Cl)ccc12] | 300 |
| 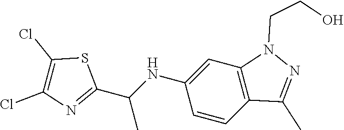 [SMILES: Cc1nn(CCO)c2cc(NC(C)c3nc(Cl)c(Cl)s3)ccc12] | 301 |
| 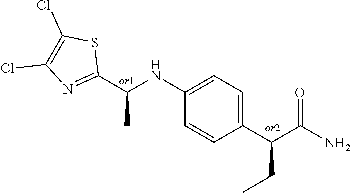 [SMILES: CC[C@H](C(N)=O)c1ccc(N[C@@H](C)c2nc(Cl)c(Cl)s2)cc1] | 302 |
| 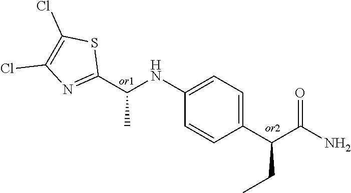 [SMILES: CC[C@H](C(N)=O)c1ccc(N[C@H](C)c2nc(Cl)c(Cl)s2)cc1] | 302A |
| 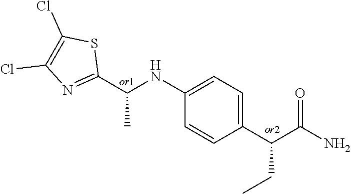 [SMILES: CC[C@@H](C(N)=O)c1ccc(N[C@H](C)c2nc(Cl)c(Cl)s2)cc1] | 302B |
| 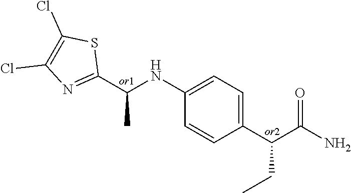 [SMILES: CC[C@@H](C(N)=O)c1ccc(N[C@@H](C)c2nc(Cl)c(Cl)s2)cc1] | 302C |
| 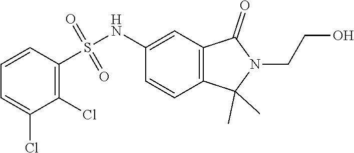 [SMILES: CC1(C)c2ccc(NS(=O)(=O)c3cccc(Cl)c3Cl)cc2C(=O)N1CCO] | 303 |
| 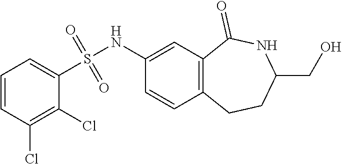 [SMILES: O=C1NC(CO)CCc2ccc(NS(=O)(=O)c3cccc(Cl)c3Cl)cc21] | 304A |
| 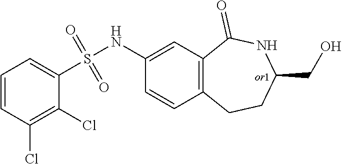 [SMILES: O=C1N[C@@H](CO)CCc2ccc(NS(=O)(=O)c3cccc(Cl)c3Cl)cc21] | 304 |
| 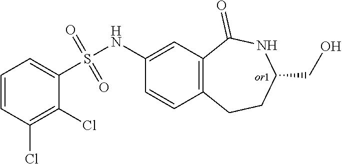 [SMILES: O=C1N[C@H](CO)CCc2ccc(NS(=O)(=O)c3cccc(Cl)c3Cl)cc21] | 304B |
| 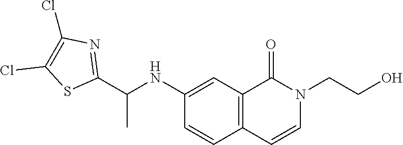 [SMILES: CC(Nc1ccc2ccn(CCO)c(=O)c2c1)c1nc(Cl)c(Cl)s1] | 305 |
| 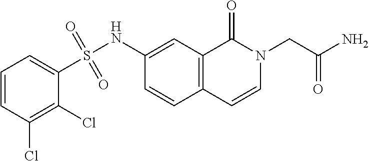 [SMILES: NC(=O)Cn1ccc2ccc(NS(=O)(=O)c3cccc(Cl)c3Cl)cc2c1=O] | 306 |
| 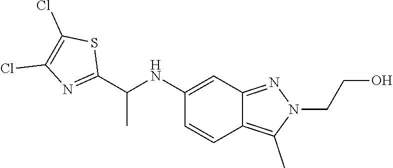 [SMILES: Cc1c2ccc(NC(C)c3nc(Cl)c(Cl)s3)cc2nn1CCO] | 307 |
| 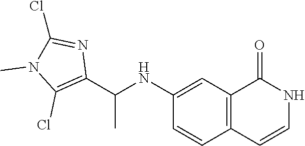 [SMILES: CC(Nc1ccc2cc[nH]c(=O)c2c1)c1nc(Cl)n(C)c1Cl] | 308 |
| 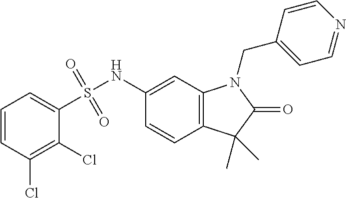 [SMILES: CC1(C)C(=O)N(Cc2ccncc2)c2cc(NS(=O)(=O)c3cccc(Cl)c3Cl)ccc21] | 309 |
| 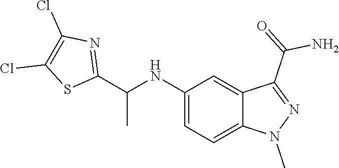 [SMILES: CC(Nc1ccc2c(c1)c(C(N)=O)nn2C)c1nc(Cl)c(Cl)s1] | 310 |
| 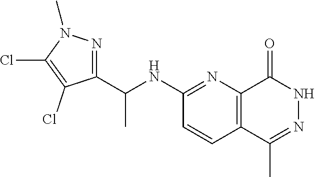 [SMILES: Cc1n[nH]c(=O)c2nc(NC(C)c3nn(C)c(Cl)c3Cl)ccc12] | 311 |
| 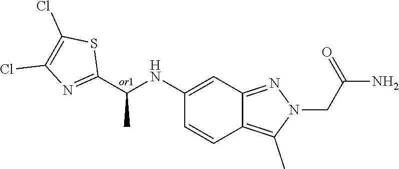 [SMILES: Cc1c2ccc(N[C@@H](C)c3nc(Cl)c(Cl)s3)cc2nn1CC(N)=O] | 312 |
| 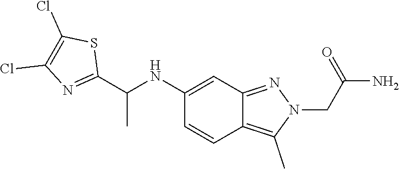 [SMILES: Cc1c2ccc(NC(C)c3nc(Cl)c(Cl)s3)cc2nn1CC(N)=O] | 312A |
| 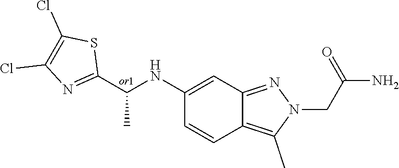 [SMILES: Cc1c2ccc(N[C@H](C)c3nc(Cl)c(Cl)s3)cc2nn1CC(N)=O] | 312B |
| 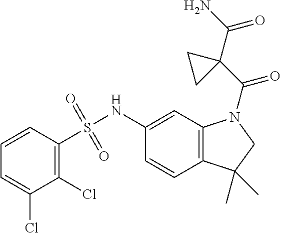 [SMILES: CC1(C)CN(C(=O)C2(C(N)=O)CC2)c2cc(NS(=O)(=O)c3cccc(Cl)c3Cl)ccc21] | 313 |
| 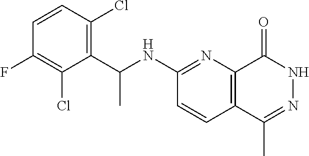 [SMILES: Cc1n[nH]c(=O)c2nc(NC(C)c3c(Cl)ccc(F)c3Cl)ccc12] | 314 |
| 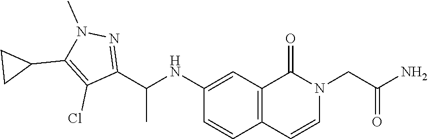 [SMILES: CC(Nc1ccc2ccn(CC(N)=O)c(=O)c2c1)c1nn(C)c(C2CC2)c1Cl] | 315 |
| 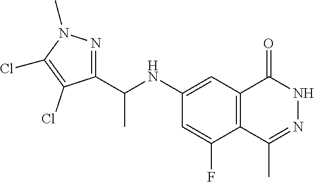 [SMILES: Cc1n[nH]c(=O)c2cc(NC(C)c3nn(C)c(Cl)c3Cl)cc(F)c12] | 316 |
| 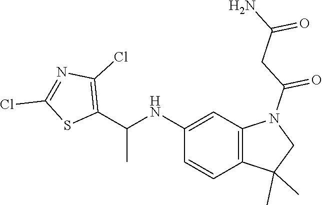 [SMILES: CC(Nc1ccc2c(c1)N(C(=O)CC(N)=O)CC2(C)C)c1sc(Cl)nc1Cl] | 317 |
| 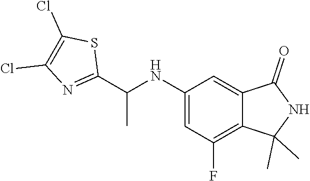 [SMILES: CC(Nc1cc(F)c2c(c1)C(=O)NC2(C)C)c1nc(Cl)c(Cl)s1] | 318 |
| 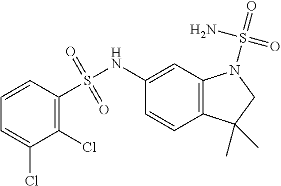 [SMILES: CC1(C)CN(S(N)(=O)=O)c2cc(NS(=O)(=O)c3cccc(Cl)c3Cl)ccc21] | 319 |
| 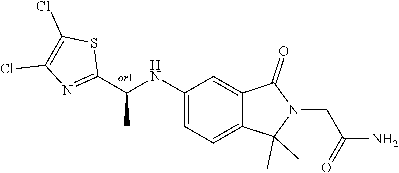 [SMILES: C[C@H](Nc1ccc2c(c1)C(=O)N(CC(N)=O)C2(C)C)c1nc(Cl)c(Cl)s1] | 320 |
| 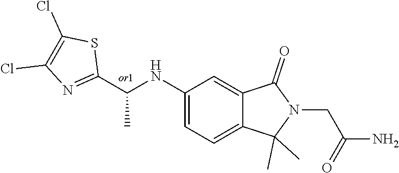 [SMILES: C[C@@H](Nc1ccc2c(c1)C(=O)N(CC(N)=O)C2(C)C)c1nc(Cl)c(Cl)s1] | 320A |
| 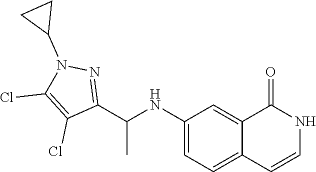 [SMILES: CC(Nc1ccc2cc[nH]c(=O)c2c1)c1nn(C2CC2)c(Cl)c1Cl] | 321 |
| 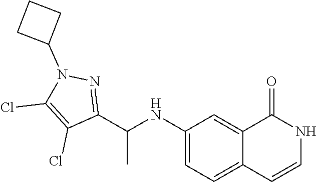 [SMILES: CC(Nc1ccc2cc[nH]c(=O)c2c1)c1nn(C2CCC2)c(Cl)c1Cl] | 322 |
| 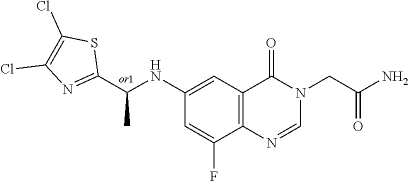 [SMILES: C[C@H](Nc1cc(F)c2ncn(CC(N)=O)c(=O)c2c1)c1nc(Cl)c(Cl)s1] | 323 |
| 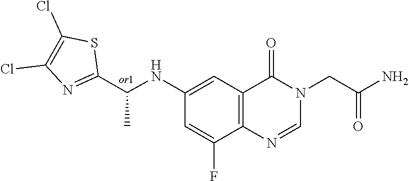 [SMILES: C[C@@H](Nc1cc(F)c2ncn(CC(N)=O)c(=O)c2c1)c1nc(Cl)c(Cl)s1] | 323A |
| 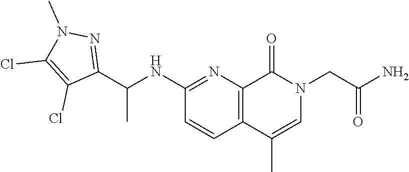 [SMILES: Cc1cn(CC(N)=O)c(=O)c2nc(NC(C)c3nn(C)c(Cl)c3Cl)ccc12] | 324 |
| 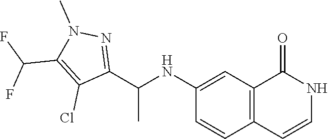 [SMILES: CC(Nc1ccc2cc[nH]c(=O)c2c1)c1nn(C)c(C(F)F)c1Cl] | 325 |
| 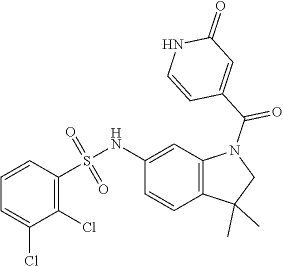 [SMILES: CC1(C)CN(C(=O)c2cc[nH]c(=O)c2)c2cc(NS(=O)(=O)c3cccc(Cl)c3Cl)ccc21] | 326 |
| 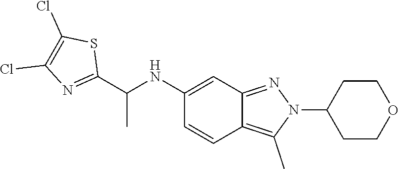 [SMILES: Cc1c2ccc(NC(C)c3nc(Cl)c(Cl)s3)cc2nn1C1CCOCC1] | 327 |
| 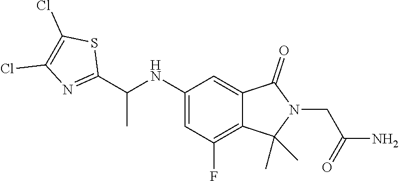 [SMILES: CC(Nc1cc(F)c2c(c1)C(=O)N(CC(N)=O)C2(C)C)c1nc(Cl)c(Cl)s1] | 328 |
| 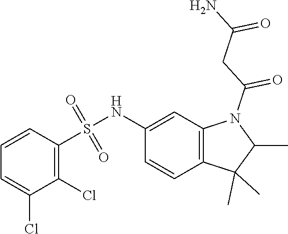 [SMILES: CC1N(C(=O)CC(N)=O)c2cc(NS(=O)(=O)c3cccc(Cl)c3Cl)ccc2C1(C)C] | 329 |
| 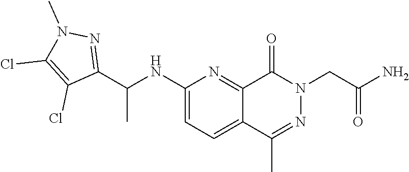 [SMILES: Cc1nn(CC(N)=O)c(=O)c2nc(NC(C)c3nn(C)c(Cl)c3Cl)ccc12] | 330 |
| 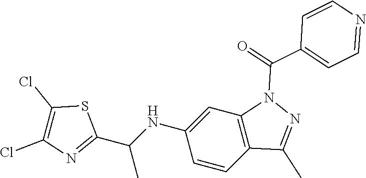 [SMILES: Cc1nn(C(=O)c2ccncc2)c2cc(NC(C)c3nc(Cl)c(Cl)s3)ccc12] | 331 |
| 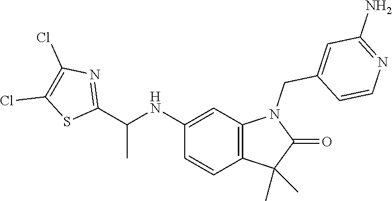 [SMILES: CC(Nc1ccc2c(c1)N(Cc1ccnc(N)c1)C(=O)C2(C)C)c1nc(Cl)c(Cl)s1] | 332 |
| 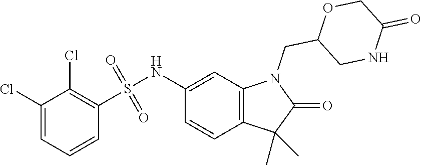 [SMILES: CC1(C)C(=O)N(CC2CNC(=O)CO2)c2cc(NS(=O)(=O)c3cccc(Cl)c3Cl)ccc21] | 333 |
| 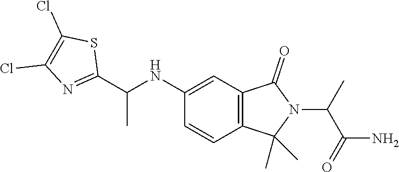 [SMILES: CC(Nc1ccc2c(c1)C(=O)N(C(C)C(N)=O)C2(C)C)c1nc(Cl)c(Cl)s1] | 334 |
| 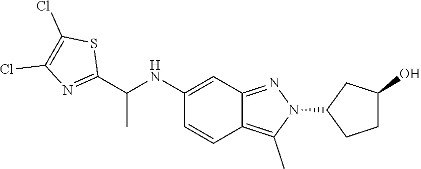 | 335 |
| 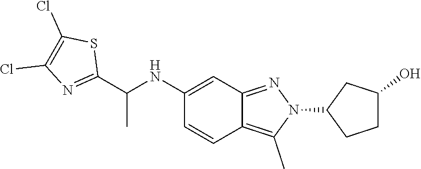 | 336 |
| 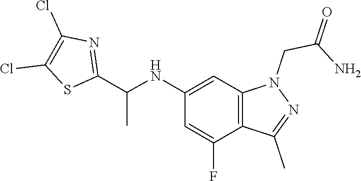 [SMILES: Cc1nn(CC(N)=O)c2cc(NC(C)c3nc(Cl)c(Cl)s3)cc(F)c12] | 337 |
| 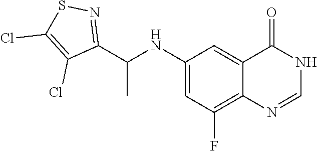 [SMILES: CC(Nc1cc(F)c2nc[nH]c(=O)c2c1)c1nsc(Cl)c1Cl] | 338 |
| 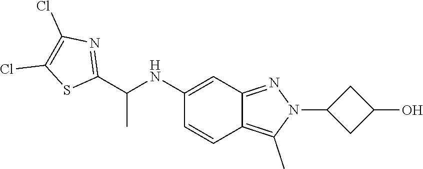 [SMILES: Cc1c2ccc(NC(C)c3nc(Cl)c(Cl)s3)cc2nn1C1CC(O)C1] | 339 |
| 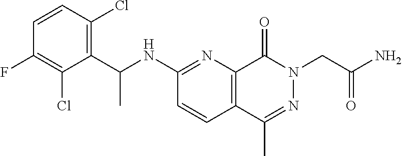 [SMILES: Cc1nn(CC(N)=O)c(=O)c2nc(NC(C)c3c(Cl)ccc(F)c3Cl)ccc12] | 340 |
| 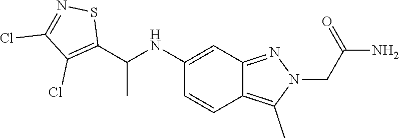 [SMILES: Cc1c2ccc(NC(C)c3snc(Cl)c3Cl)cc2nn1CC(N)=O] | 341 |
| 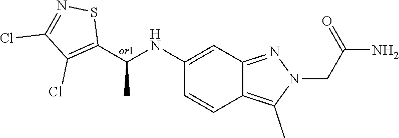 [SMILES: Cc1c2ccc(N[C@@H](C)c3snc(Cl)c3Cl)cc2nn1CC(N)=O] | 341A |
| 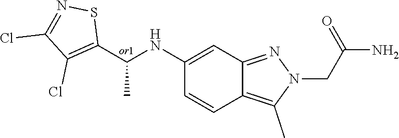 [SMILES: Cc1c2ccc(N[C@H](C)c3snc(Cl)c3Cl)cc2nn1CC(N)=O] | 341B |
| 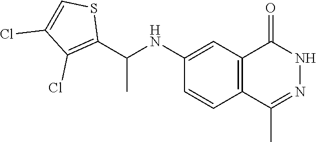 [SMILES: Cc1n[nH]c(=O)c2cc(NC(C)c3scc(Cl)c3Cl)ccc12] | 342 |
| 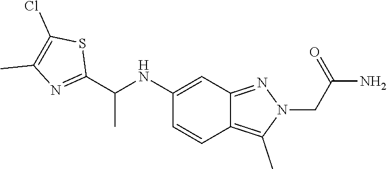 [SMILES: Cc1nc(C(C)Nc2ccc3c(C)n(CC(N)=O)nc3c2)sc1Cl] | 343 |
| 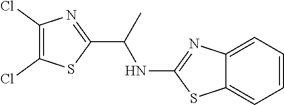 [SMILES: CC(Nc1nc2ccccc2s1)c1nc(Cl)c(Cl)s1] | 344 |
| 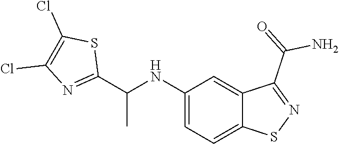 [SMILES: CC(Nc1ccc2snc(C(N)=O)c2c1)c1nc(Cl)c(Cl)s1] | 345 |
| 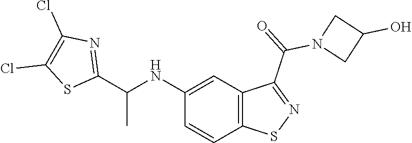 [SMILES: CC(Nc1ccc2snc(C(=O)N3CC(O)C3)c2c1)c1nc(Cl)c(Cl)s1] | 346 |
| 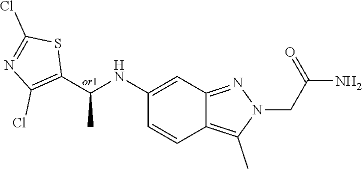 [SMILES: Cc1c2ccc(N[C@@H](C)c3sc(Cl)nc3Cl)cc2nn1CC(N)=O] | 347 |
| 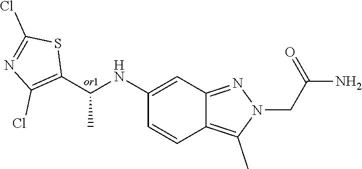 [SMILES: Cc1c2ccc(N[C@H](C)c3sc(Cl)nc3Cl)cc2nn1CC(N)=O] | 347A |
| 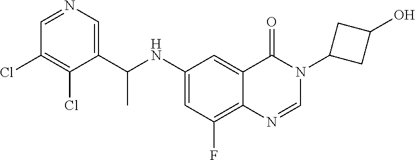 [SMILES: CC(Nc1cc(F)c2ncn(C3CC(O)C3)c(=O)c2c1)c1cncc(Cl)c1Cl] | 348 |
| 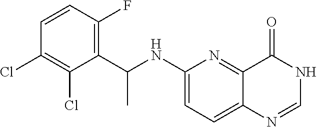 [SMILES: CC(Nc1ccc2nc[nH]c(=O)c2n1)c1c(F)ccc(Cl)c1Cl] | 349 |
| 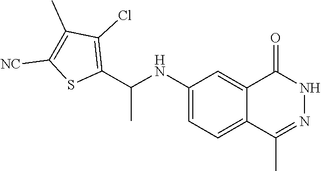 [SMILES: Cc1c(C#N)sc(C(C)Nc2ccc3c(C)n[nH]c(=O)c3c2)c1Cl] | 350 |
| 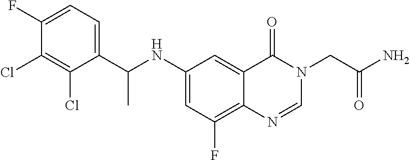 [SMILES: CC(Nc1cc(F)c2ncn(CC(N)=O)c(=O)c2c1)c1ccc(F)c(Cl)c1Cl] | 351 |
| 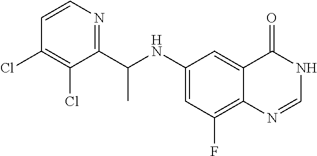 [SMILES: CC(Nc1cc(F)c2nc[nH]c(=O)c2c1)c1nccc(Cl)c1Cl] | 352 |
| 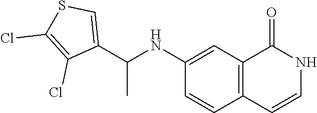 [SMILES: CC(Nc1ccc2cc[nH]c(=O)c2c1)c1csc(Cl)c1Cl] | 353 |
| 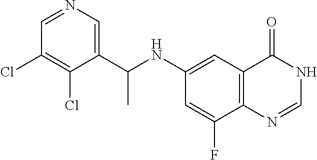 [SMILES: CC(Nc1cc(F)c2nc[nH]c(=O)c2c1)c1cncc(Cl)c1Cl] | 354 |
| 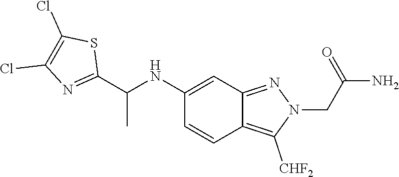 [SMILES: CC(Nc1ccc2c(C(F)F)n(CC(N)=O)nc2c1)c1nc(Cl)c(Cl)s1] | 355 |
| 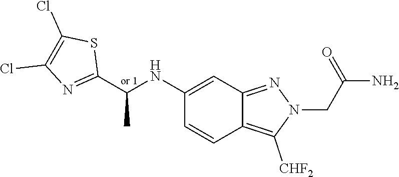 [SMILES: C[C@H](Nc1ccc2c(C(F)F)n(CC(N)=O)nc2c1)c1nc(Cl)c(Cl)s1] | 355A |
| 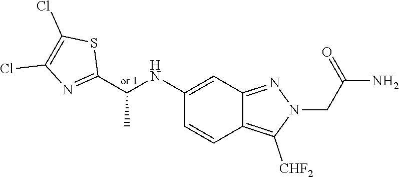 [SMILES: C[C@@H](Nc1ccc2c(C(F)F)n(CC(N)=O)nc2c1)c1nc(Cl)c(Cl)s1] | 355B |
| 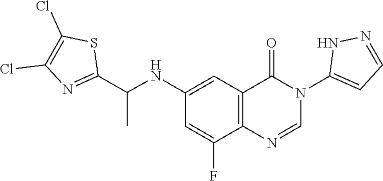 [SMILES: CC(Nc1cc(F)c2ncn(-c3ccn[nH]3)c(=O)c2c1)c1nc(Cl)c(Cl)s1] | 356-rac |
| 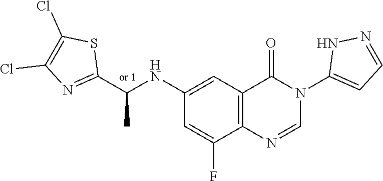 [SMILES: C[C@H](Nc1cc(F)c2ncn(-c3ccn[nH]3)c(=O)c2c1)c1nc(Cl)c(Cl)s1] | 356 |
| 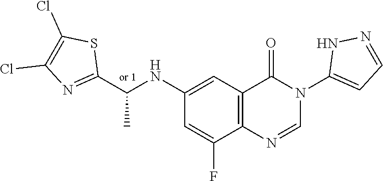 [SMILES: C[C@@H](Nc1cc(F)c2ncn(-c3ccn[nH]3)c(=O)c2c1)c1nc(Cl)c(Cl)s1] | 356A |
| 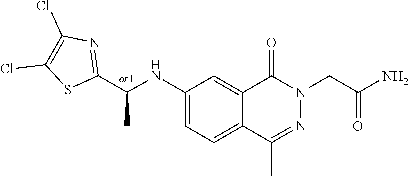 [SMILES: Cc1nn(CC(N)=O)c(=O)c2cc(N[C@@H](C)c3nc(Cl)c(Cl)s3)ccc12] | 357 |
| 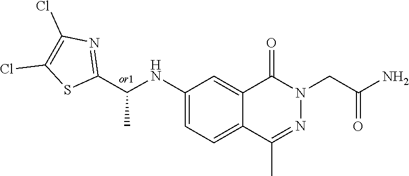 [SMILES: Cc1nn(CC(N)=O)c(=O)c2cc(N[C@H](C)c3nc(Cl)c(Cl)s3)ccc12] | 358 |
| 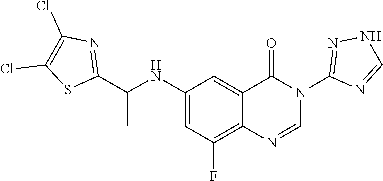 [SMILES: CC(Nc1cc(F)c2ncn(-c3nc[nH]n3)c(=O)c2c1)c1nc(Cl)c(Cl)s1] | 359 |
| 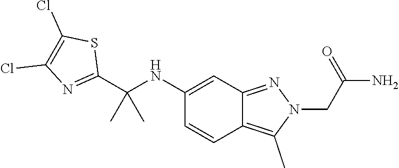 [SMILES: Cc1c2ccc(NC(C)(C)c3nc(Cl)c(Cl)s3)cc2nn1CC(N)=O] | 360 |
| 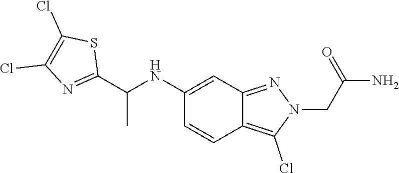 [SMILES: CC(Nc1ccc2c(Cl)n(CC(N)=O)nc2c1)c1nc(Cl)c(Cl)s1] | 361 |
| 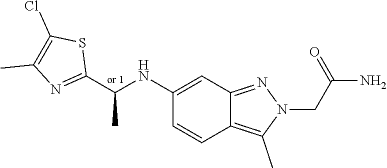 [SMILES: Cc1nc([C@H](C)Nc2ccc3c(C)n(CC(N)=O)nc3c2)sc1Cl] | 362 |
| 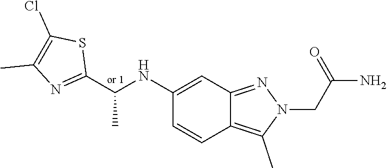 [SMILES: Cc1nc([C@@H](C)Nc2ccc3c(C)n(CC(N)=O)nc3c2)sc1Cl] | 363 |
| 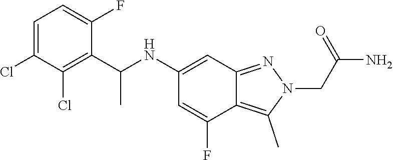 [SMILES: Cc1c2c(F)cc(NC(C)c3c(F)ccc(Cl)c3Cl)cc2nn1CC(N)=O] | 364 |
| 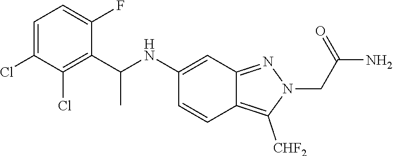 [SMILES: CC(Nc1ccc2c(C(F)F)n(CC(N)=O)nc2c1)c1c(F)ccc(Cl)c1Cl] | 365 |
| 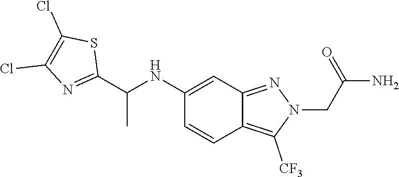 [SMILES: CC(Nc1ccc2c(C(F)(F)F)n(CC(N)=O)nc2c1)c1nc(Cl)c(Cl)s1] | 366 |
| 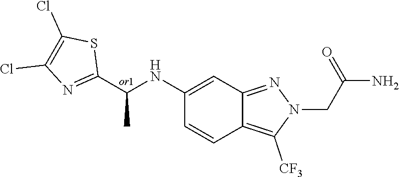 [SMILES: C[C@H](Nc1ccc2c(C(F)(F)F)n(CC(N)=O)nc2c1)c1nc(Cl)c(Cl)s1] | 367 |
| 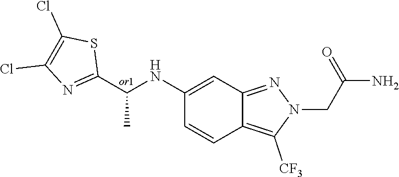 [SMILES: C[C@@H](Nc1ccc2c(C(F)(F)F)n(CC(N)=O)nc2c1)c1nc(Cl)c(Cl)s1] | 368 |
| 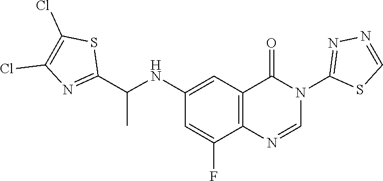 [SMILES: CC(Nc1cc(F)c2ncn(-c3nncs3)c(=O)c2c1)c1nc(Cl)c(Cl)s1] | 369 |
| 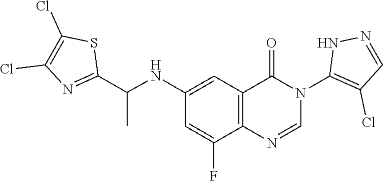 [SMILES: CC(Nc1cc(F)c2ncn(-c3[nH]ncc3Cl)c(=O)c2c1)c1nc(Cl)c(Cl)s1] | 370 |
| 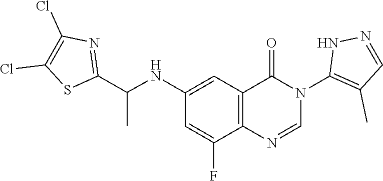 [SMILES: Cc1cn[nH]c1-n1cnc2c(F)cc(NC(C)c3nc(Cl)c(Cl)s3)cc2c1=O] | 371 |
| 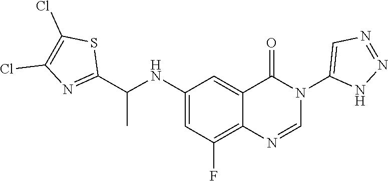 [SMILES: CC(Nc1cc(F)c2ncn(-c3cnn[nH]3)c(=O)c2c1)c1nc(Cl)c(Cl)s1] | 372 |
| 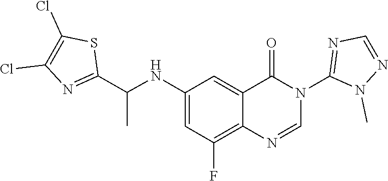 [SMILES: CC(Nc1cc(F)c2ncn(-c3ncnn3C)c(=O)c2c1)c1nc(Cl)c(Cl)s1] | 373 |
| 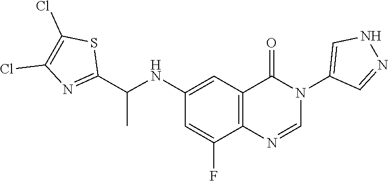 [SMILES: CC(Nc1cc(F)c2ncn(-c3cn[nH]c3)c(=O)c2c1)c1nc(Cl)c(Cl)s1] | 374 |
| 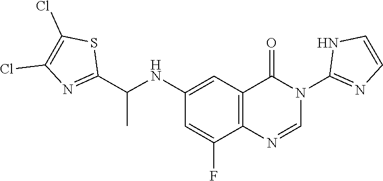 [SMILES: CC(Nc1cc(F)c2ncn(-c3ncc[nH]3)c(=O)c2c1)c1nc(Cl)c(Cl)s1] | 375 |
| 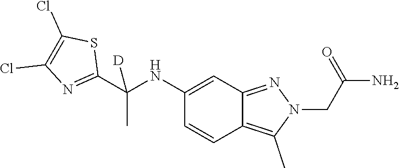 [SMILES: [2H]C(C)(Nc1ccc2c(C)n(CC(N)=O)nc2c1)c1nc(Cl)c(Cl)s1] | 376 |
| 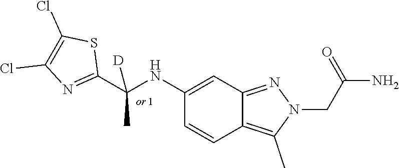 [SMILES: [2H][C@](C)(Nc1ccc2c(C)n(CC(N)=O)nc2c1)c1nc(Cl)c(Cl)s1] | 377 |
| 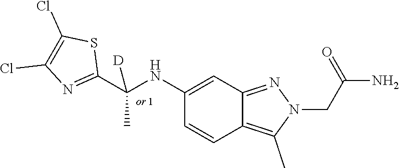 [SMILES: [2H][C@@](C)(Nc1ccc2c(C)n(CC(N)=O)nc2c1)c1nc(Cl)c(Cl)s1] | 378 |
| 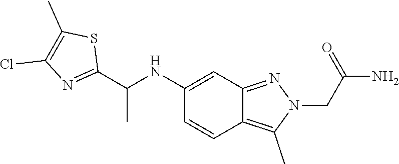 [SMILES: Cc1sc(C(C)Nc2ccc3c(C)n(CC(N)=O)nc3c2)nc1Cl] | 379 |
| 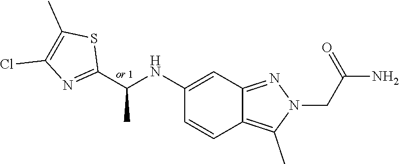 [SMILES: Cc1sc([C@H](C)Nc2ccc3c(C)n(CC(N)=O)nc3c2)nc1Cl] | 380 |
| 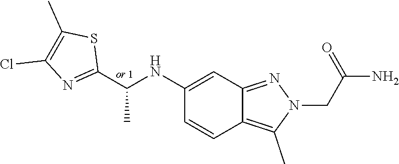 [SMILES: Cc1sc([C@@H](C)Nc2ccc3c(C)n(CC(N)=O)nc3c2)nc1Cl] | 381 |
| 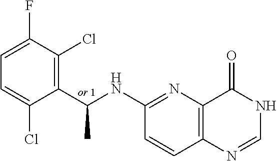 [SMILES: C[C@H](Nc1ccc2nc[nH]c(=O)c2n1)c1c(Cl)ccc(F)c1Cl] | 382 |
| 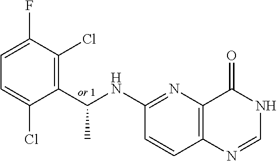 [SMILES: C[C@@H](Nc1ccc2nc[nH]c(=O)c2n1)c1c(Cl)ccc(F)c1Cl] | 383 |
| 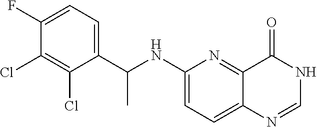 [SMILES: CC(Nc1ccc2nc[nH]c(=O)c2n1)c1ccc(F)c(Cl)c1Cl] | 384 |
| 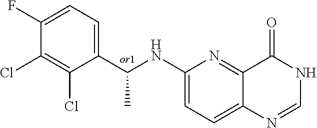 [SMILES: C[C@@H](Nc1ccc2nc[nH]c(=O)c2n1)c1ccc(F)c(Cl)c1Cl] | 385 |
| 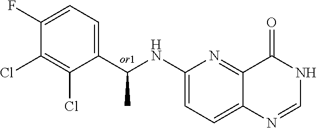 [SMILES: C[C@H](Nc1ccc2nc[nH]c(=O)c2n1)c1ccc(F)c(Cl)c1Cl] | 386 |
| 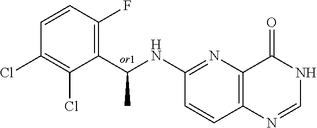 [SMILES: C[C@H](Nc1ccc2nc[nH]c(=O)c2n1)c1c(F)ccc(Cl)c1Cl] | 387 |
| 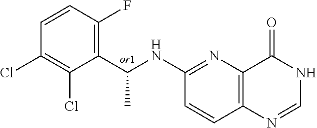 [SMILES: C[C@@H](Nc1ccc2nc[nH]c(=O)c2n1)c1c(F)ccc(Cl)c1Cl] | 388 |
| 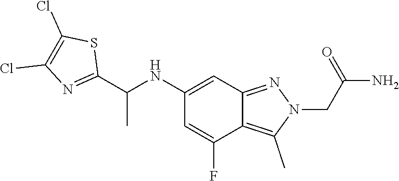 [SMILES: Cc1c2c(F)cc(NC(C)c3nc(Cl)c(Cl)s3)cc2nn1CC(N)=O] | 389 |
| 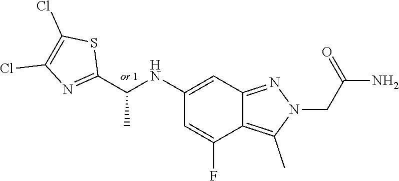 [SMILES: Cc1c2c(F)cc(N[C@H](C)c3nc(Cl)c(Cl)s3)cc2nn1CC(N)=O] | 390 |
| 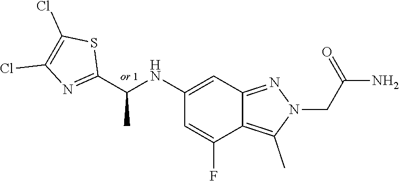 [SMILES: Cc1c2c(F)cc(N[C@@H](C)c3nc(Cl)c(Cl)s3)cc2nn1CC(N)=O] | 391 |

The notation “or1” or “or2” as used in chemical structures herein mean that the stereo configuration of the chiral center labeled by “or1” or “or2” is of either R-configuration or S-configuration. For example, Compounds 194A and 194B has an “or1” positioned above the stereo center attached to a methyl group and a hydrogen atom (hydrogen atom not shown). This means that the stereo configuration of the chiral carbon in Compounds 194A and 194B labeled by “or1” is either R or S. Single stereoisomers, i.e., a compound having an R-configuration and a compound having an S-configuration, were separately obtained and characterized by NMR, MS, and retention time, but the stereo configuration of the chiral center was not determined. In other words, each of the compounds

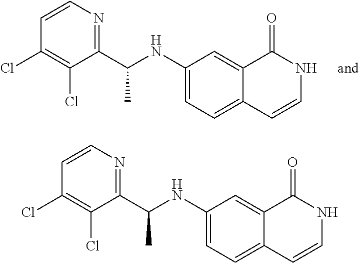 [SMILES: C[C@@H](Nc1ccc2cc[nH]c(=O)c2c1)c1nccc(Cl)c1Cl ; "and" ; C[C@H](Nc1ccc2cc[nH]c(=O)c2c1)c1nccc(Cl)c1Cl]

was separately obtained as a single stereoisomer. In such cases, the compounds (e.g., Compounds 194A and 194B) are indicated as “Single Unknown Stereoisomer.”

For another example, Compounds 302, 302A, 302B, and 302C have an “or1” positioned above one stereo center and an “or2” positioned above another stereo center. This means that the stereo configuration of the two chiral carbons in these compounds labeled by “or1” and “or2” are respectively either R or S. Single stereoisomers, e.g., a compound having an (S, S) configuration, a compound having an (R, S) configuration, a compound having an (R, R) configuration, and a compound having an (S, R) configuration, were separately obtained and characterized by NMR, MS, and retention time, but the stereo configuration of the chiral centers was not determined. In other words, each of the compounds

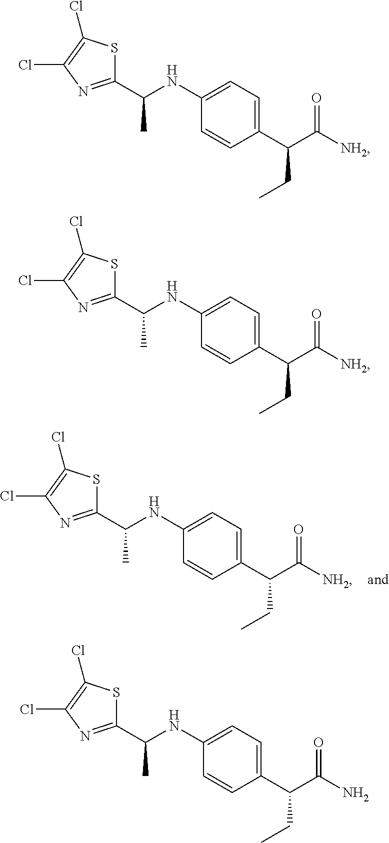 [SMILES: CC[C@H](C(N)=O)c1ccc(N[C@@H](C)c2nc(Cl)c(Cl)s2)cc1 ; CC[C@H](C(N)=O)c1ccc(N[C@H](C)c2nc(Cl)c(Cl)s2)cc1 ; CC[C@@H](C(N)=O)c1ccc(N[C@H](C)c2nc(Cl)c(Cl)s2)cc1 ; "and" ; CC[C@@H](C(N)=O)c1ccc(N[C@@H](C)c2nc(Cl)c(Cl)s2)cc1]

separately obtained as a single stereoisomer.

In certain embodiments, e.g., in embodiments of various uses and methods, a compound of the disclosure is selected from Compounds 1A to 121A depicted in Table 2, a tautomer thereof, a solvate or stereoisomer of the compound or the tautomer, or a pharmaceutically acceptable salt of the foregoing.

TABLE 2
Compounds 1A to 121A

| | |
|---|---|
| 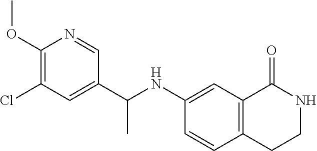 [SMILES: COc1ncc(C(C)Nc2ccc3c(c2)C(=O)NCC3)cc1Cl] | 1A |
| (CAS#: 2249192-79-6) | |
| 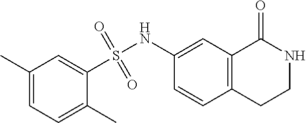 [SMILES: Cc1ccc(C)c(S(=O)(=O)Nc2ccc3c(c2)C(=O)NCC3)c1] | 2A |
| (CAS#: 1705439-70-8) | |
| 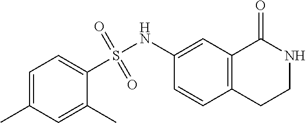 [SMILES: Cc1ccc(S(=O)(=O)Nc2ccc3c(c2)C(=O)NCC3)c(C)c1] | 3A |
| (CAS#: 1704547-29-4) | |
| 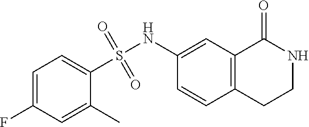 [SMILES: Cc1cc(F)ccc1S(=O)(=O)Nc1ccc2c(c1)C(=O)NCC2] | 4A |
| (CAS#: 1705313-64-9) | |
| 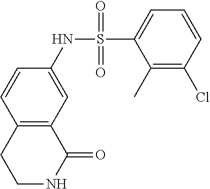 [SMILES: Cc1c(Cl)cccc1S(=O)(=O)Nc1ccc2c(c1)C(=O)NCC2] | 5A |
| (CAS#: 1351658-768) | |
| 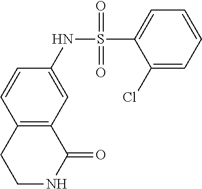 [SMILES: O=C1NCCc2ccc(NS(=O)(=O)c3ccccc3Cl)cc21] | 6A |
| (CAS#: 1706359-50-3) | |
| 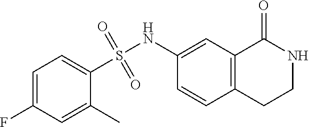 [SMILES: Cc1cc(F)ccc1S(=O)(=O)Nc1ccc2c(c1)C(=O)NCC2] | 7A |
| (CAS#: 2454280-07-8) | |
| 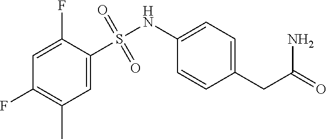 [SMILES: Cc1cc(S(=O)(=O)Nc2ccc(CC(N)=O)cc2)c(F)cc1F] | 8A |
| (CAS#: 1427646-07-8) | |
| 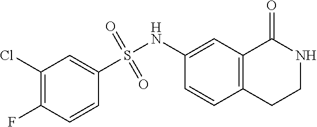 [SMILES: O=C1NCCc2ccc(NS(=O)(=O)c3ccc(F)c(Cl)c3)cc21] | 9A |
| (CAS#: 1705418-11-6) | |
| 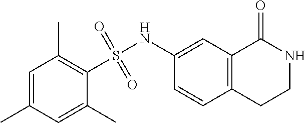 [SMILES: Cc1cc(C)c(S(=O)(=O)Nc2ccc3c(c2)C(=O)NCC3)c(C)c1] | 10A |
| (CAS#: 1706279-52-8) | |
| 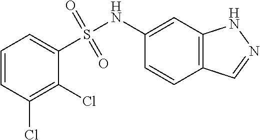 [SMILES: O=S(=O)(Nc1ccc2cn[nH]c2c1)c1cccc(Cl)c1Cl] | 11A |
| (CAS#: 778621-41-3) | |
| 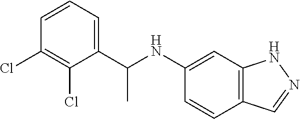 [SMILES: CC(Nc1ccc2cn[nH]c2c1)c1cccc(Cl)c1Cl] | 12A |
| (CAS#: 1962748-80-6) | |
| 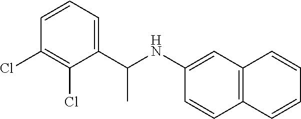 [SMILES: CC(Nc1ccc2ccccc2c1)c1cccc(Cl)c1Cl] | 13A |
| (CAS#: 1944445-67-3) | |
| 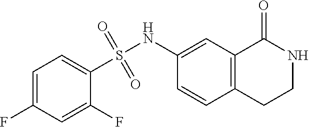 [SMILES: O=C1NCCc2ccc(NS(=O)(=O)c3ccc(F)cc3F)cc21] | 14A |
| (CAS#: 1705360-93-5) | |
| 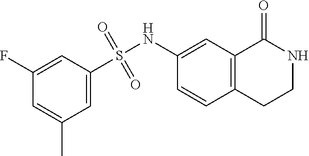 [SMILES: Cc1cc(F)cc(S(=O)(=O)Nc2ccc3c(c2)C(=O)NCC3)c1] | 15A |
| (CAS#: 1427966-15-1) | |
| 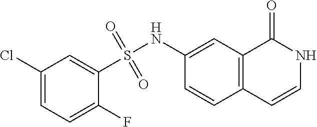 [SMILES: O=c1[nH]ccc2ccc(NS(=O)(=O)c3cc(Cl)ccc3F)cc12] | 16A |
| (CAS#: 2728615-72-1) | |
| 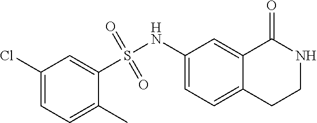 [SMILES: Cc1ccc(Cl)cc1S(=O)(=O)Nc1ccc2c(c1)C(=O)NCC2] | 17A |
| (CAS#: 1706279-68-6) | |
| 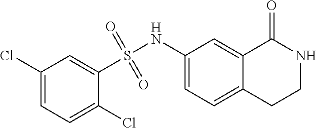 [SMILES: O=C1NCCc2ccc(NS(=O)(=O)c3cc(Cl)ccc3Cl)cc21] | 18A |
| (CAS#: 1705439-87-7) | |
| 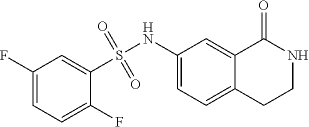 [SMILES: O=C1NCCc2ccc(NS(=O)(=O)c3cc(F)ccc3F)cc21] | 19A |
| (CAS#: 1705418-03-6) | |
| 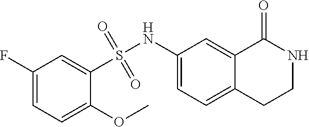 [SMILES: COc1ccc(F)cc1S(=O)(=O)Nc1ccc2c(c1)C(=O)NCC2] | 20A |
| (CAS#: 1705944-56-4) | |
| 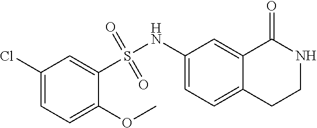 [SMILES: COc1ccc(Cl)cc1S(=O)(=O)Nc1ccc2c(c1)C(=O)NCC2] | 21A |
| (CAS#: 1705439-77-5) | |
| 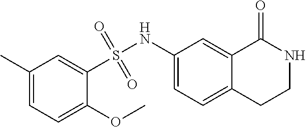 [SMILES: COc1ccc(C)cc1S(=O)(=O)Nc1ccc2c(c1)C(=O)NCC2] | 22A |
| (CAS#: 1704547-35-2) | |
| 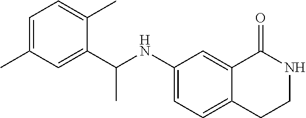 [SMILES: Cc1ccc(C)c(C(C)Nc2ccc3c(c2)C(=O)NCC3)c1] | 23A |
| (CAS#: 2434477-39-9) | |
| 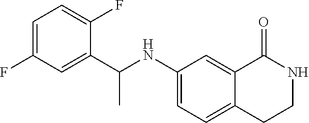 [SMILES: CC(Nc1ccc2c(c1)C(=O)NCC2)c1cc(F)ccc1F] | 24A |
| (CAS#: 2450195-53-4) | |
| 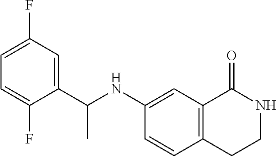 [SMILES: CC(Nc1ccc2c(c1)C(=O)NCC2)c1cc(F)ccc1F] | 25A |
| (CAS#: 2433443-40-2) | |
| 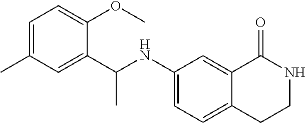 [SMILES: COc1ccc(C)cc1C(C)Nc1ccc2c(c1)C(=O)NCC2] | 26A |
| (CAS#: 2457156-85-1) | |
| 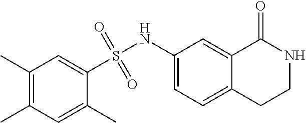 [SMILES: Cc1cc(C)c(S(=O)(=O)Nc2ccc3c(c2)C(=O)NCC3)cc1C] | 27A |
| (CAS#: 1705188-07-3) | |
| 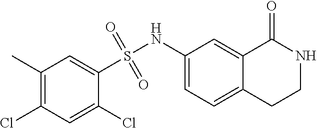 [SMILES: Cc1cc(S(=O)(=O)Nc2ccc3c(c2)C(=O)NCC3)c(Cl)cc1Cl] | 28A |
| (CAS#: 1705313-79-6) | |
| 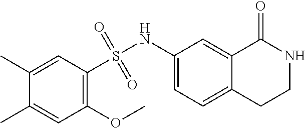 [SMILES: COc1cc(C)c(C)cc1S(=O)(=O)Nc1ccc2c(c1)C(=O)NCC2] | 29A |
| (CAS#: 1706023-62-2) | |
| 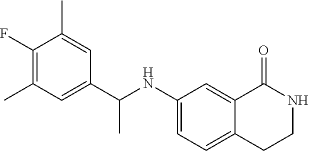 [SMILES: Cc1cc(C(C)Nc2ccc3c(c2)C(=O)NCC3)cc(C)c1F] | 30A |
| (CAS#: 2460895-07-0) | |
| 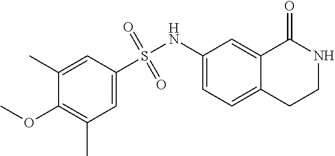 [SMILES: COc1c(C)cc(S(=O)(=O)Nc2ccc3c(c2)C(=O)NCC3)cc1C] | 31A |
| (CAS#: 1704991-25-2) | |
| 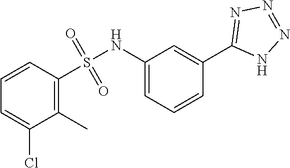 [SMILES: Cc1c(Cl)cccc1S(=O)(=O)Nc1cccc(-c2nnn[nH]2)c1] | 32A |
| (CAS#: 1365720-05-3) | |
| 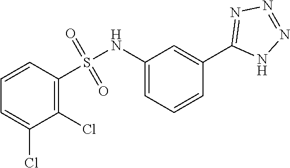 [SMILES: O=S(=O)(Nc1cccc(-c2nnn[nH]2)c1)c1cccc(Cl)c1Cl] | 33A |
| (CAS#: 1365720-00-8) | |
| 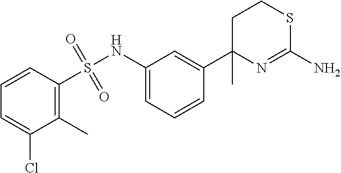 [SMILES: Cc1c(Cl)cccc1S(=O)(=O)Nc1cccc(C2(C)CCSC(N)=N2)c1] | 34A |
| (CAS#: 935985-65-2) | |
| 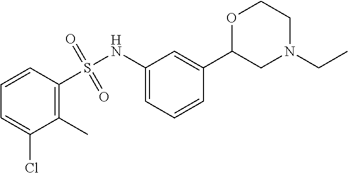 [SMILES: CCN1CCOC(c2cccc(NS(=O)(=O)c3cccc(Cl)c3C)c2)C1] | 35A |
| (CAS#: 1010383-89-7) | |
| 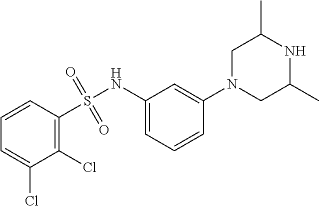 [SMILES: CC1CN(c2cccc(NS(=O)(=O)c3cccc(Cl)c3Cl)c2)CC(C)N1] | 36A |
| (CAS#: 874956-54-4) | |
| 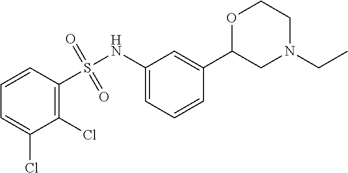 [SMILES: CCN1CCOC(c2cccc(NS(=O)(=O)c3cccc(Cl)c3Cl)c2)C1] | 37A |
| 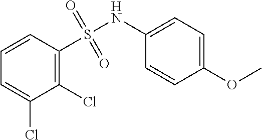 [SMILES: COc1ccc(NS(=O)(=O)c2cccc(Cl)c2Cl)cc1] | 38A |
| (CAS#: 82417-11-6) | |
| 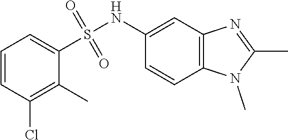 [SMILES: Cc1c(Cl)cccc1S(=O)(=O)Nc1ccc2c(c1)nc(C)n2C] | 39A |
| (CAS#: 686746-69-0) | |
| 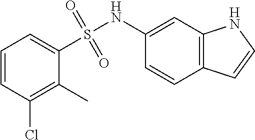 [SMILES: Cc1c(Cl)cccc1S(=O)(=O)Nc1ccc2cc[nH]c2c1] | 40A |
| (CAS#: 686746-92-9) | |
| 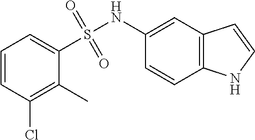 [SMILES: Cc1c(Cl)cccc1S(=O)(=O)Nc1ccc2[nH]ccc2c1] | 41A |
| (CAS#: 686747-18-2) | |
| 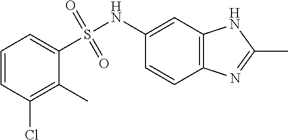 [SMILES: Cc1nc2ccc(NS(=O)(=O)c3cccc(Cl)c3C)cc2[nH]1] | 42A |
| (CAS#: 686746-83-8) | |
| 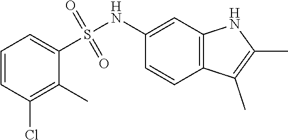 [SMILES: Cc1[nH]c2cc(NS(=O)(=O)c3cccc(Cl)c3C)ccc2c1C] | 43A |
| (CAS#: 686746-75-8) | |
| 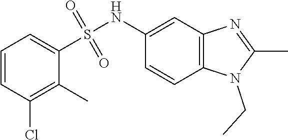 [SMILES: CCn1c(C)nc2cc(NS(=O)(=O)c3cccc(Cl)c3C)ccc21] | 44A |
| (CAS#: 686747-33-1) | |
| 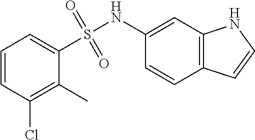 [SMILES: Cc1c(Cl)cccc1S(=O)(=O)Nc1ccc2cc[nH]c2c1] | 45A |
| (CAS#: 851774-58-8) | |
| 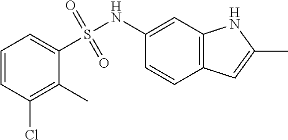 [SMILES: Cc1cc2ccc(NS(=O)(=O)c3cccc(Cl)c3C)cc2[nH]1] | 46A |
| (CAS#: 686747-17-1) | |
| 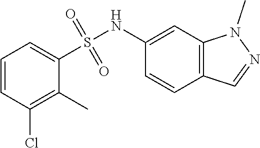 [SMILES: Cc1c(Cl)cccc1S(=O)(=O)Nc1ccc2cnn(C)c2c1] | 47A |
| (CAS#: 851774-59-9) | |
| 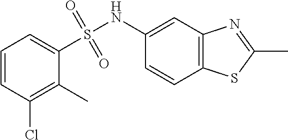 [SMILES: Cc1nc2cc(NS(=O)(=O)c3cccc(Cl)c3C)ccc2s1] | 48A |
| (CAS#: 686746-31-6) | |
| 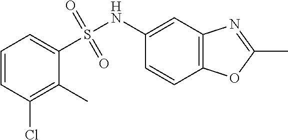 [SMILES: Cc1nc2cc(NS(=O)(=O)c3cccc(Cl)c3C)ccc2o1] | 49A |
| (CAS#: 686747-45-5) | |
| 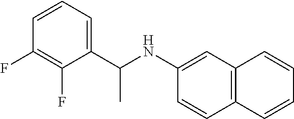 [SMILES: CC(Nc1ccc2ccccc2c1)c1cccc(F)c1F] | 50A |
| (CAS#: 1963323-33-2) | |
| 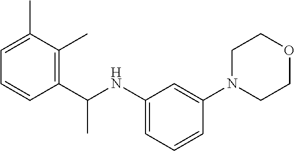 [SMILES: Cc1cccc(C(C)Nc2cccc(N3CCOCC3)c2)c1C] | 51A |
| (CAS#: 2463522-30-5) | |
| 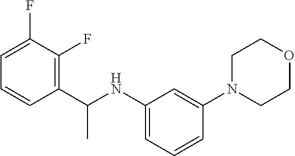 [SMILES: CC(Nc1cccc(N2CCOCC2)c1)c1cccc(F)c1F] | 52A |
| (CAS#: 2450049-83-7) | |
| 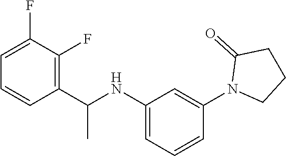 [SMILES: CC(Nc1cccc(N2CCCC2=O)c1)c1cccc(F)c1F] | 53A |
| (CAS#: 2451332-01-5) | |
| 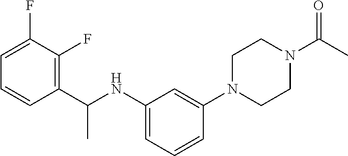 [SMILES: CC(=O)N1CCN(c2cccc(NC(C)c3cccc(F)c3F)c2)CC1] | 54A |
| (CAS#: 2430195-53-0) | |
| 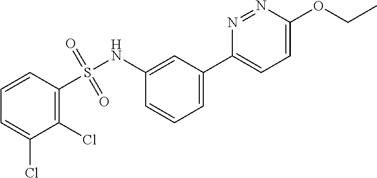 [SMILES: CCOc1ccc(-c2cccc(NS(=O)(=O)c3cccc(Cl)c3Cl)c2)nn1] | 55A |
| (CAS#: 921097-89-4) | |
| 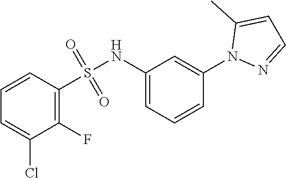 [SMILES: Cc1ccnn1-c1cccc(NS(=O)(=O)c2cccc(Cl)c2F)c1] | 56A |
| (CAS#: 1318542-71-0) | |
| 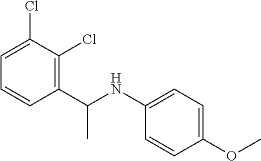 [SMILES: COc1ccc(NC(C)c2cccc(Cl)c2Cl)cc1] | 57A |
| (CAS#: 2115300-12-2) | |
| 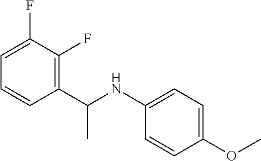 [SMILES: COc1ccc(NC(C)c2cccc(F)c2F)cc1] | 58A |
| (CAS#: 1916871-35-6) | |
| 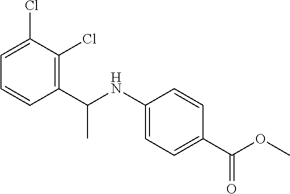 [SMILES: COC(=O)c1ccc(NC(C)c2cccc(Cl)c2Cl)cc1] | 59A |
| (CAS#: 1963647-36-0) | |
| 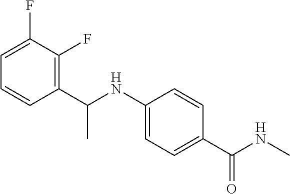 [SMILES: CNC(=O)c1ccc(NC(C)c2cccc(F)c2F)cc1] | 60A |
| (CAS#: 1963025-89-9) | |
| 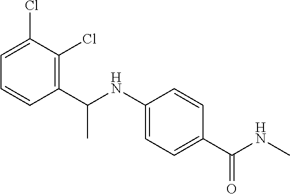 [SMILES: CNC(=O)c1ccc(NC(C)c2cccc(Cl)c2Cl)cc1] | 61A |
| (CAS#: 1929754-55-1) | |
| 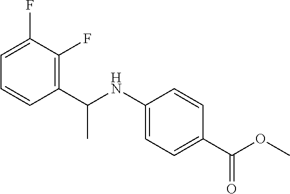 [SMILES: COC(=O)c1ccc(NC(C)c2cccc(F)c2F)cc1] | 62A |
| (CAS#: 1963025-78-6) | |
| 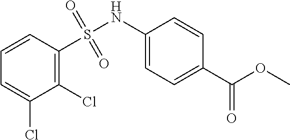 [SMILES: COC(=O)c1ccc(NS(=O)(=O)c2cccc(Cl)c2Cl)cc1] | 63A |
| (CAS#: 785788-55-8) | |
| 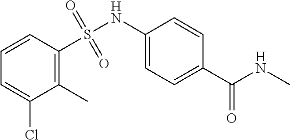 [SMILES: CNC(=O)c1ccc(NS(=O)(=O)c2cccc(Cl)c2C)cc1] | 64A |
| (CAS#: 1387358-31-7) | |
| 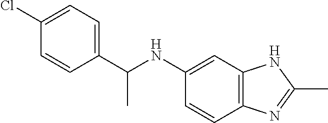 [SMILES: Cc1nc2ccc(NC(C)c3ccc(Cl)cc3)cc2[nH]1] | 65A |
| (CAS#: 1157743-00-4) | |
| 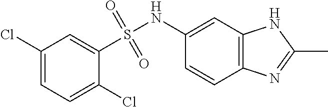 [SMILES: Cc1nc2ccc(NS(=O)(=O)c3cc(Cl)ccc3Cl)cc2[nH]1] | 66A |
| (CAS#: 1232798-27-4) | |
| 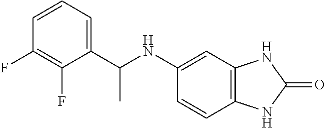 [SMILES: CC(Nc1ccc2[nH]c(=O)[nH]c2c1)c1cccc(F)c1F] | 67A |
| (CAS#: 1939247-11-6) | |
| 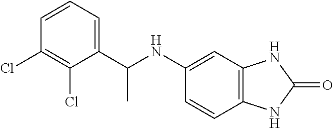 [SMILES: CC(Nc1ccc2[nH]c(=O)[nH]c2c1)c1cccc(Cl)c1Cl] | 68A |
| (CAS#: 1937182-90-5) | |
| 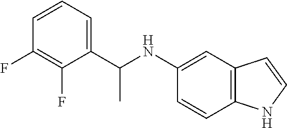 [SMILES: CC(Nc1ccc2[nH]ccc2c1)c1cccc(F)c1F] | 69A |
| (CAS#: 1963070-36-1) | |
| 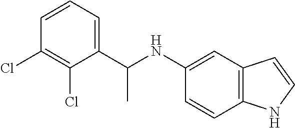 [SMILES: CC(Nc1ccc2[nH]ccc2c1)c1cccc(Cl)c1Cl] | 70A |
| (CAS#: 1933996-67-8) | |
| 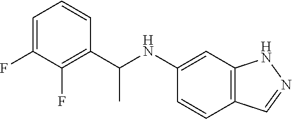 [SMILES: CC(Nc1ccc2cn[nH]c2c1)c1cccc(F)c1F] | 71A |
| (CAS#: 1934013-16-7) | |
| 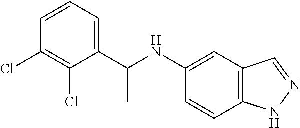 [SMILES: CC(Nc1ccc2[nH]ncc2c1)c1cccc(Cl)c1Cl] | 72A |
| (CAS#: 1933996-75-8) | |
| 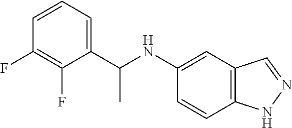 [SMILES: CC(Nc1ccc2[nH]ncc2c1)c1cccc(F)c1F] | 73A |
| (CAS#: 1916021-89-0) | |
| 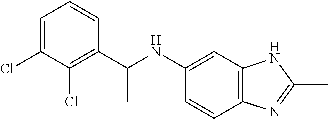 [SMILES: Cc1nc2ccc(NC(C)c3cccc(Cl)c3Cl)cc2[nH]1] | 74A |
| (CAS#: 1963742-47-3) | |
| 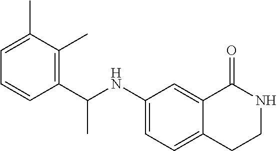 [SMILES: Cc1cccc(C(C)Nc2ccc3c(c2)C(=O)NCC3)c1C] | 75A |
| (CAS#: 2467467-72-5) | |
| 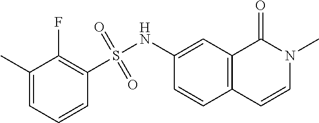 | 76A |
| (CAS#: 2728281-62-5) | |
| 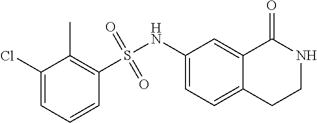 [SMILES: Cc1c(Cl)cccc1S(=O)(=O)Nc1ccc2c(c1)C(=O)NCC2] | 77A |
| (CAS#: 1351658-76-8) | |
| 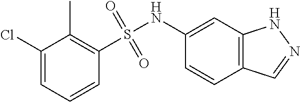 [SMILES: Cc1c(Cl)cccc1S(=O)(=O)Nc1ccc2cn[nH]c2c1] | 78A |
| (CAS#: 2326619-66-1) | |
| 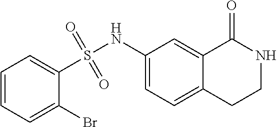 [SMILES: O=C1NCCc2ccc(NS(=O)(=O)c3ccccc3Br)cc21] | 79A |
| (CAS#: 1705361-40-5) | |
| 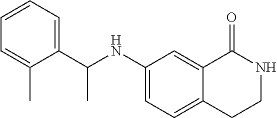 [SMILES: Cc1ccccc1C(C)Nc1ccc2c(c1)C(=O)NCC2] | 80A |
| (CAS#: 2467870-05-7) | |
| 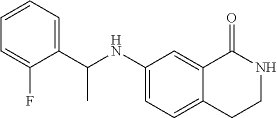 [SMILES: CC(Nc1ccc2c(c1)C(=O)NCC2)c1ccccc1F] | 81A |
| (CAS#: 2461467-89-8) | |
| 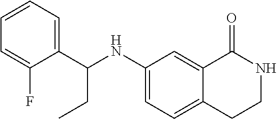 [SMILES: CCC(Nc1ccc2c(c1)C(=O)NCC2)c1ccccc1F] | 82A |
| (CAS#: 2461340-71-4) | |
| 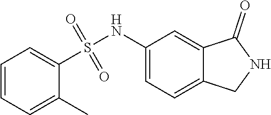 [SMILES: Cc1ccccc1S(=O)(=O)Nc1ccc2c(c1)C(=O)NC2] | 83A |
| (CAS#: 2456292-95-6) | |
| 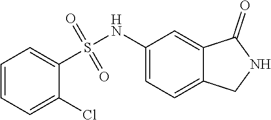 [SMILES: O=C1NCc2ccc(NS(=O)(=O)c3ccccc3Cl)cc21] | 84A |
| (CAS#: 2463451-33-2) | |
| 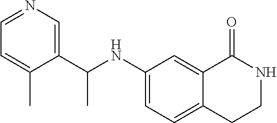 [SMILES: Cc1ccncc1C(C)Nc1ccc2c(c1)C(=O)NCC2] | 85A |
| (CAS#: 2463870-01-9) | |
| 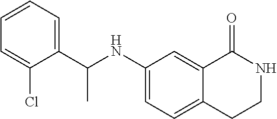 [SMILES: CC(Nc1ccc2c(c1)C(=O)NCC2)c1ccccc1Cl] | 86A |
| (CAS#: 2435425-21-9) | |
| 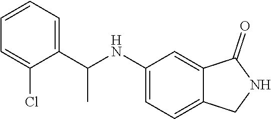 [SMILES: CC(Nc1ccc2c(c1)C(=O)NC2)c1ccccc1Cl] | 87A |
| (CAS#: 2425500-88-3) | |
| 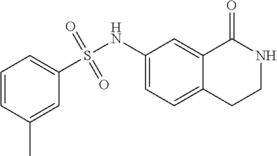 [SMILES: Cc1cccc(S(=O)(=O)Nc2ccc3c(c2)C(=O)NCC3)c1] | 88A |
| (CAS#: 1705641-11-7) | |
| 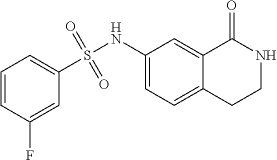 [SMILES: O=C1NCCc2ccc(NS(=O)(=O)c3cccc(F)c3)cc21] | 89A |
| (CAS#: 1706279-50-6) | |
| 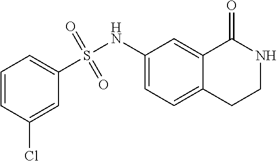 [SMILES: O=C1NCCc2ccc(NS(=O)(=O)c3cccc(Cl)c3)cc21] | 90A |
| (CAS#: 1705188-00-6) | |
| 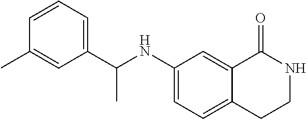 [SMILES: Cc1cccc(C(C)Nc2ccc3c(c2)C(=O)NCC3)c1] | 91A |
| (CAS#: 2433301-05-2) | |
| 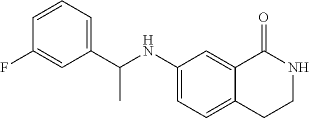 [SMILES: CC(Nc1ccc2c(c1)C(=O)NCC2)c1cccc(F)c1] | 91A |
| (CAS#: 433301-04-1) | |
| 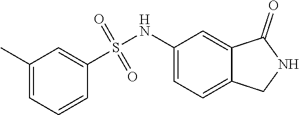 [SMILES: Cc1cccc(S(=O)(=O)Nc2ccc3c(c2)C(=O)NC3)c1] | 92A |
| (CAS#: 2448968-23-6) | |
| 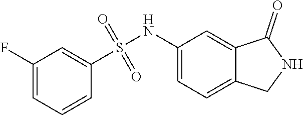 [SMILES: O=C1NCc2ccc(NS(=O)(=O)c3cccc(F)c3)cc21] | 93A |
| (CAS#: 2433477-59-7) | |
| 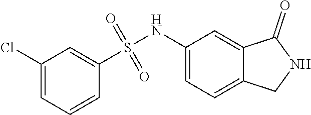 [SMILES: O=C1NCc2ccc(NS(=O)(=O)c3cccc(Cl)c3)cc21] | 94A |
| (CAS#: 2467402-45-3) | |
| 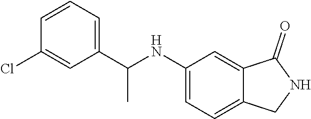 [SMILES: CC(Nc1ccc2c(c1)C(=O)NC2)c1cccc(Cl)c1] | 95A |
| (CAS#: 2454645-36-2) | |
| 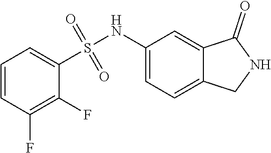 [SMILES: O=C1NCc2ccc(NS(=O)(=O)c3cccc(F)c3F)cc21] | 96A |
| (CAS#: 2466848-89-3) | |
| 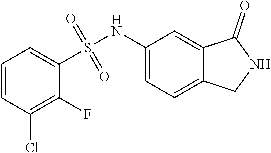 [SMILES: O=C1NCc2ccc(NS(=O)(=O)c3cccc(Cl)c3F)cc21] | 97A |
| (CAS#: 2465216-83-3) | |
| 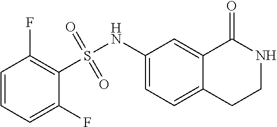 [SMILES: O=C1NCCc2ccc(NS(=O)(=O)c3c(F)cccc3F)cc21] | 98A |
| (CAS#: 1704990-27-1) | |
| 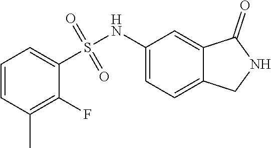 [SMILES: Cc1cccc(S(=O)(=O)Nc2ccc3c(c2)C(=O)NC3)c1F] | 99A |
| (CAS#: 2464812-26-6) | |
| 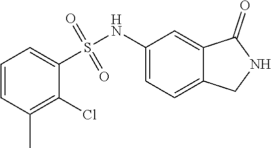 [SMILES: Cc1cccc(S(=O)(=O)Nc2ccc3c(c2)C(=O)NC3)c1Cl] | 100A |
| (CAS#: 2460506-04-9) | |
| 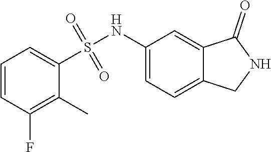 [SMILES: Cc1c(F)cccc1S(=O)(=O)Nc1ccc2c(c1)C(=O)NC2] | 101A |
| (CAS#: 2460011-37-2) | |
| 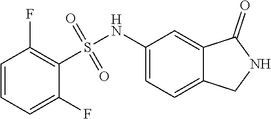 [SMILES: O=C1NCc2ccc(NS(=O)(=O)c3c(F)cccc3F)cc21] | 102A |
| (CAS#: 2461809-09-4) | |
| 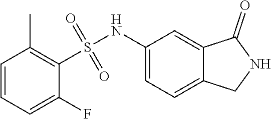 [SMILES: Cc1cccc(F)c1S(=O)(=O)Nc1ccc2c(c1)C(=O)NC2] | 103A |
| (CAS#: 2464869-08-5) | |
| 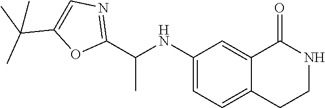 [SMILES: CC(Nc1ccc2c(c1)C(=O)NCC2)c1ncc(C(C)(C)C)o1] | 104A |
| (CAS#: 1436179-02-0) | |
| 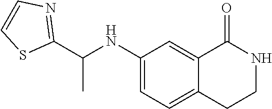 [SMILES: CC(Nc1ccc2c(c1)C(=O)NCC2)c1nccs1] | 105A |
| (CAS#: 2464319-24-0) | |
| 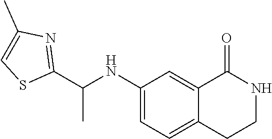 [SMILES: Cc1csc(C(C)Nc2ccc3c(c2)C(=O)NCC3)n1] | 106A |
| (CAS#: 2461452-75-3) | |
| 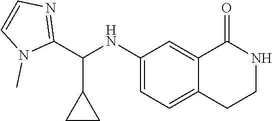 [SMILES: Cn1ccnc1C(Nc1ccc2c(c1)C(=O)NCC2)C1CC1] | 107A |
| (CAS#: 2461789-68-2) | |
| 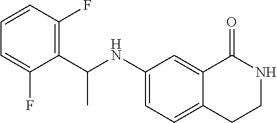 [SMILES: CC(Nc1ccc2c(c1)C(=O)NCC2)c1c(F)cccc1F] | 108A |
| (CAS#: 2430423-95-1) | |
| 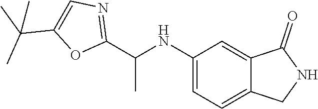 [SMILES: CC(Nc1ccc2c(c1)C(=O)NC2)c1ncc(C(C)(C)C)o1] | 109A |
| (CAS#: 2194389-53-0) | |
| 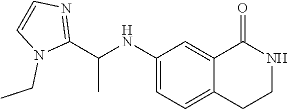 [SMILES: CCn1ccnc1C(C)Nc1ccc2c(c1)C(=O)NCC2] | 110A |
| (CAS#: 2449757-98-4) | |
| 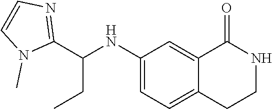 [SMILES: CCC(Nc1ccc2c(c1)C(=O)NCC2)c1nccn1C] | 111A |
| (CAS#: 2450211-67-1) | |
| 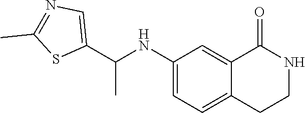 [SMILES: Cc1ncc(C(C)Nc2ccc3c(c2)C(=O)NCC3)s1] | 112A |
| (CAS#: 2434051-62-2) | |
| 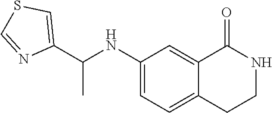 [SMILES: CC(Nc1ccc2c(c1)C(=O)NCC2)c1cscn1] | 113A |
| (CAS#: 2465605-86-9) | |
| 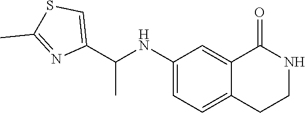 [SMILES: Cc1nc(C(C)Nc2ccc3c(c2)C(=O)NCC3)cs1] | 114A |
| (CAS#: 2432819-70-8) | |
| 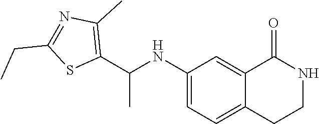 [SMILES: CCc1nc(C)c(C(C)Nc2ccc3c(c2)C(=O)NCC3)s1] | 115A |
| (CAS#: 2465762-26-7) | |
| 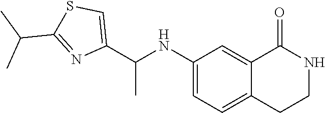 [SMILES: CC(C)c1nc(C(C)Nc2ccc3c(c2)C(=O)NCC3)cs1] | 116A |
| (CAS#: 2461992-25-4) | |
| 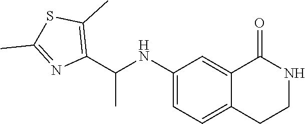 [SMILES: Cc1nc(C(C)Nc2ccc3c(c2)C(=O)NCC3)c(C)s1] | 117A |
| (CAS#: 2464318-95-2) | |
| 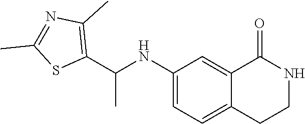 [SMILES: Cc1nc(C)c(C(C)Nc2ccc3c(c2)C(=O)NCC3)s1] | 118A |
| (CAS#: 2451525-94-1) | |
| 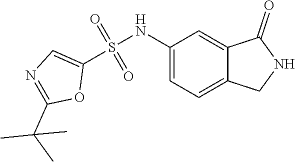 [SMILES: CC(C)(C)c1ncc(S(=O)(=O)Nc2ccc3c(c2)C(=O)NC3)o1] | 119A |
| (CAS#: 2761522-62-5) | |
| 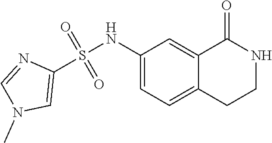 [SMILES: Cn1cnc(S(=O)(=O)Nc2ccc3c(c2)C(=O)NCC3)c1] | 120A |
| (CAS#: 1428373-70-9) | |
| 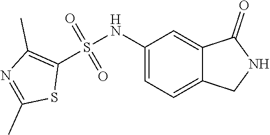 [SMILES: Cc1nc(C)c(S(=O)(=O)Nc2ccc3c(c2)C(=O)NC3)s1] | 121A |
| (CAS#: 2467254-04-0) | |

Another aspect of the disclosure provides a pharmaceutical composition comprising at least one compound selected from a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391) and Compounds 1A to 121A in Table 2 disclosed herein, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A in Table 2 disclosed herein, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A in Table 2 disclosed herein, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used in the aforementioned form or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

The present disclosure provides methods of treatment and uses utilizing a compound set forth in any one of the various embodiments of Section II (Compounds and Compositions) and Tables 1 and 2, e.g., a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3, as well as Compounds 1 to 391 in Table 1 and Compounds 1A to 121A in Table 2, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

One aspect of the disclosure provides a method of treating a disease or condition, comprising administering to a subject in need thereof, a therapeutically effective amount of a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but not limited to taxanes, vinca alkaloids and proteasome inhibitors. In some embodiments, the disease or condition is caused by axonal degeneration or neuronal cells damage.

In another aspect, disclosed herein is a compound, tautomer, solvate, stereoisomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for use as a medicament.

In another aspect, disclosed herein is use of a compound, tautomer, solvate, stereoisomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for treating a disease or condition that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cell damage.

In a further aspect of this disclosure, a compound, tautomer, solvate, stereoisomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in treating a disease or condition that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cell damage.

Another aspect of the disclosure provides a method of inhibiting or preventing axonal degeneration, comprising administering to a subject in need thereof, a therapeutically effective amount of a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for inhibiting or preventing axonal degeneration or neuronal cells damage.

In a further aspect of this disclosure, a compound, tautomer, solvate, stereoisomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in inhibiting or preventing axonal degeneration or neuronal cells damage.

Another aspect of the disclosure provides a method of modulating, e.g., inhibiting, SARM1 in a subject in need thereof, comprising administering to the subject, a therapeutically effective amount of a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for modulating, e.g., inhibiting, SARM1 in a subject in need thereof.

In another aspect of this disclosure, a compound, tautomer, a solvate or stereoisomer of the compound or the tautomer, or pharmaceutically acceptable salt as described herein, including a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in modulating, e.g., inhibiting, SARM1 in a subject in need thereof by contacting the subject with the compound, tautomer, a solvate or stereoisomer of the compound or the tautomer, pharmaceutically acceptable salt, or pharmaceutical composition.

A compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered once daily, twice daily, or three times daily, for example, for the treatment of a disease or condition, that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by axonal degeneration or neuronal cells damage.

A compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein. Generally, administration is adjusted to achieve a therapeutic dosage of about 0.1 to 50 mg/kg, preferably 0.5 to 10 mg/kg, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of Formulae 1, 2, 2a, 2b, 2c, 2d, 2e, 2f, 2g, 2h, 2i, 2j, 2k, 2L, 2m, 3, 3a, 3b, 3c, 3d, 3e, 3f, 3g, 3h, 3i, 3j, 3k, 3L, 4, 5, 6, 7, 7a, 7b, 8, 8a, 9, 9a, 10, 11, 12, 13, 13a, 13b, 14, 14a, 14b, 14c, 14d, 14e, 14f, 14g, 14h-1, 14h-2, 14i, 14j, 15, 15a, 16-1, 16-2, 16-1a, 16-2a, 17, 17a, 18, 18a, 19, 20, 21, 21a, 21b, 22, 22a, 23, 23a, 24, 25, 26, 27, 28-1, 28-2, 29-1, 29-2, 29-3, 30-1, 30-2, 30-3, 30-4, 30-5, 31-1, 31-2, 32-1, 32-2, 32-3, 32-4, 32-5, 33-1, 33-2, 33-3, 34-1, 34-2, 34-3, 34-4, 34-5, 34-6, 35-1, 35-2, 35-3, 35-4, 36-1, 36-2, 36-3, 37-1, 37-2, 37-3, 37-4, 37-5, 37-6, 38-1, 38-2, 38-3, 39-1, 39-2, 39-3, 39-4, 40-1, 40-2, 40-3, 41-1, 41-2, 41-3, 41-4, 41-5, 42-1, 42-2, 42-3, 43-1, 43-2, 43-3, 43-4, 44-1, 44-2, 44-3, 44-4, 44-5, 44-6, 44-7, 44-8, 44-9, 44-10, 44-11, 44-12, 44-13, 44-14, 45-1, 45-2, 46-1, 46-2, and 46-3 (e.g., Compounds 1 to 391), and Compounds 1A to 121A disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt are administered once daily, twice daily, or three times daily. The compound, tautomer, solvate, stereoisomer, or pharmaceutically acceptable salt described herein is administered for morning/daytime dosing, with off period at night.

### A. EXAMPLES

In order that the disclosure described herein may be more fully understood, the following examples are disclosed herein. It should be understood that these examples are for illustrative purposes only and are not to be construed as limiting this disclosure in any way.

### Example I. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following general synthetic procedures and specific synthetic schemes for Compounds 1 to 354 and Compound 1A to 15A as representative examples of Formula 1.

### Preparation of (5-(3,5-difluorophenyl)-4,5-dihydro-1H-pyrazol-1-yl)(3-((6-fluoropyridin-3-yl)oxy)azetidine-1-yl)methanone (1) Method 1

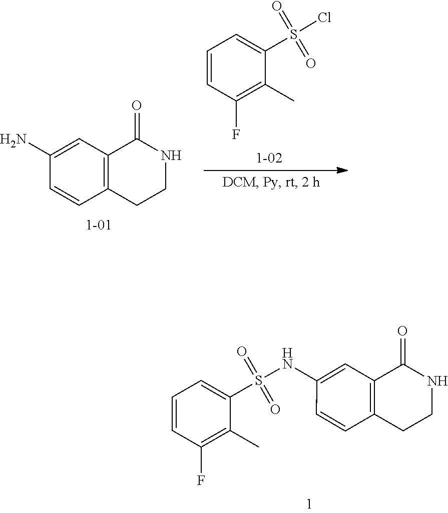 [SMILES: Nc1ccc2c(c1)C(=O)NCC2 ; Cc1c(F)cccc1S(=O)(=O)Cl ; Cc1c(F)cccc1S(=O)(=O)Nc1ccc2c(c1)C(=O)NCC2]

To a solution of 7-amino-3,4-dihydroisoquinolin-1(2H)-one 1-01 (150 mg, 0.92 mmol) in DCM (10 mL) was added 3-fluoro-2-methylbenzenesulfonyl chloride 1-02 (192 mg, 0.92 mmol), Pyridine (219 mg, 2.7 mmol). The mixture was stirred at 25° C. for 2 hours. Water was added and the resulting mixture was extracted with EtOAc, dried over Na₂SO₄, and concentrated in vacuo. Purification by silica gel chromatography gave Compound 1 (90 mg, 27.6%) as a white solid. Mass (m/z): 335.40 [M+H]⁺. ¹H NMR (400 MHz, DMSO-d₆) δ 10.68 (s, 1H), 7.90 (d, J=3.0 Hz, 1H), 7.70 (dd, J=7.6, 1.6 Hz, 1H), 7.57 (d, J=2.0 Hz, 1H), 7.49-7.34 (m, 2H), 7.16 (d, J=2.0 Hz, 2H), 3.28 (td, J=6.4, 2.8 Hz, 2H), 2.76 (, t, J=6.4 Hz 2H), 2.48 (d, J=2.4 Hz, 3H).

Examples (Compounds) 2-91, 191, 196-197, 204, and 206, 208-209, 211-212, 217, 219, 220, 224-225, 227, 251, 265-265, 268-270, 270A, 274, 283, 298, 304, 304A and 304B were synthesized using a method like that used in Example 1.

| Example | | | | |
|---|---|---|---|---|
| No. | | | | |
| (Compound | | Appearance | | MS |
| No.) | | and | | (m/z) |
| Method | Structure and Name | Yield | ¹H NMR Data | [M + H]⁺ |
| Example 2 Method 1 | 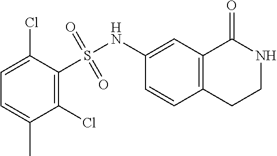 [SMILES: Cc1ccc(Cl)c(S(=O)(=O)Nc2ccc3c(c2)C(=O)NCC3)c1Cl] | red solid, yield: 30.5% | ¹H NMR (400 MHz, DMSO-d₆) δ 10.85 (s, 1H), 7.91 (s, 1H), 7.61 (d, J = 2.3 Hz, 1H), 7.59 − 7.49 (m, 2H), 7.25 − 7.15 (m, 2H), 3.31 − 3.25 (m, 2H), 2.76 (t, J = 6.6 Hz, 2H), 2.34 (s, 3H). | 385.26 |
| | 2,6-dichloro-3- | | | |
| | methyl-N-(1-oxo- | | | |
| | 3,4-dihydro-2H- | | | |
| | isoquinolin-7- | | | |
| | yl) | | | |
| | benzenesulfonamide | | | |
| Example 3 Method 1 | 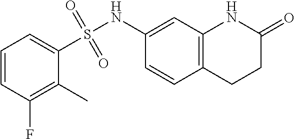 [SMILES: Cc1c(F)cccc1S(=O)(=O)Nc1ccc2c(c1)NC(=O)CC2] 3-fluoro-2-methyl-N- (2-oxo-1,2,3,4- | White solid, yield: 41.7% | ¹H NMR (400 MHz, DMSO-d₆) δ 10.46 (s, 1H), 10.01 (s, 1H), 7.72 (dd, J = 7.6, 1.6 Hz, 1H), 7.41 (m, 2H), 6.97 (d, J = 8.4 Hz, 1H), 6.68 (d, J = 2.0 Hz, 1H), 6.58 (dd, J = 8.0, 2.4 Hz, 1H), 2.72 (t, J = 7.2 Hz, 2H), 2.48 (d, J = 2.4 Hz, 3H), 2.36 (t, J = 8.4 Hz, 2H). | 335.17 |
| | tetrahydroquinolin-7- | | | |
| | yl)benzenesulfonamide | | | |
| Example 4 Method 1 | 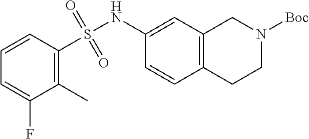 [SMILES: Cc1c(F)cccc1S(=O)(=O)Nc1ccc2c(c1)CN(C(=O)OC(C)(C)C)CC2] (tert-butyl 7-((3- | White solid, yield: 19.8% | ¹H NMR (400 MHz, DMSO-d₆) δ 10.46 (s, 1H), 7.73 (d, J = 6.8, 1H), 7.47-7.37 (m, 2H), 7.01 (d, J = 8.4 Hz, 1H), 6.89 − 6.83 (m, 2H), 4.35 (s, 2H), 3.47 (t, J = 5.6 Hz, 2H), 2.63 (t, J = 6.0, 2H), 2.48 (d, J = 2.4 Hz, 3H), 1.40 (s, 9H). | 420.5 |
| | fluoro-2- | | | |
| | methylphenyl) | | | |
| | sulfonamido)-3,4- | | | |
| | dihydroisoquinoline- | | | |
| | 2(1H)-carboxylate | | | |
| Example 5 Method 1 | 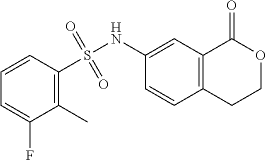 [SMILES: Cc1c(F)cccc1S(=O)(=O)Nc1ccc2c(c1)C(=O)OCC2] | White solid, yield: 5.0% | ¹H NMR (400 MHz, DMSO-d₆) δ 10.95 (s, 1H), 7.73 − 7.71 (m, 1H), 7.59 (d, J = 2.4 Hz, 1H), 7.45 − 7.38 (m, 2H), 7.29 − 7.23 (m, 2H), 4.41 (t, J = 6.0 Hz, 2H), 2.91 (t, J = 6.0 Hz, 2H), 2.48 (s, 3H). | 336.1 |
| | 3-fluoro-2-methyl-N- | | | |
| | (1-oxoisochroman-7- | | | |
| | yl)benzenesulfonamide | | | |
| Example 6 Method 1 | 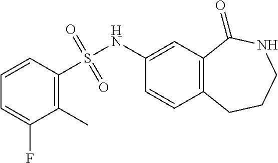 [SMILES: Cc1c(F)cccc1S(=O)(=O)Nc1ccc2c(c1)C(=O)NCCC2] | White solid, yield: 30.3% | ¹H NMR (400 MHz, DMSO-d₆) δ 10.64 (s, 1H), 8.01 (t, J = 5.6 Hz, 1H), 7.71 (dd, J = 7.6, 1.6 Hz, 1H), 7.47-7.37 (m, 2H), 7.20 (t, J = 1.6 Hz, 1H), 7.10 (d, J = 1.6 Hz, 2H), 2.81 (q, J = 6.4 Hz, 2H), 2.61 (t, J = 6.8 Hz, 2H), 2.47 (d, | 349.2 |
| | | | J = 2.4 Hz, 3H), 1.79 | |
| | 3-fluoro-2-methyl-N- | | (p, J = 6.8 Hz, 2H). | |
| | (1-oxo-2,3,4,5- | | | |
| | tetrahydro-1H- | | | |
| | benzo[c]azepin-8- | | | |
| | yl)benzenesulfonamide | | | |
| Example 7 Method 1 | 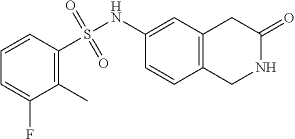 [SMILES: Cc1c(F)cccc1S(=O)(=O)Nc1ccc2c(c1)CC(=O)NC2] 3-fluoro-2-methyl-N- | White solid, yield: 19.5% | ¹H NMR (400 MHz, DMSO-d₆) δ 10.80 (s, 1H), 7.91 (s, 1H), 7.73 (dd, J = 7.2, 2.0 Hz, 1H), 7.41 − 7.33 (m, 2H), 7.06 (d, J = 8.0 Hz, 1H), 6.88 − 6.84 (m, 2H), 4.19 (s, 2H), 3.29 (s, 2H), 2.47 (d, J = 2.4 Hz, 3H). | 335.1 |
| | (3-oxo-1,2,3,4- | | | |
| | tetrahydroisoquinolin-6- | | | |
| | yl)benzenesulfonamide | | | |
| Example 8 Method 1 | 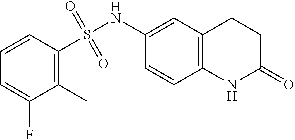 [SMILES: Cc1c(F)cccc1S(=O)(=O)Nc1ccc2c(c1)CCC(=O)N2] 3-fluoro-2-methyl-N-(2-oxo-1,2,3,4- tetrahydroquinolin-6- yl)benzenesulfonamide | White solid, yield: 41.9% | ¹H NMR (400 MHz, DMSO-d₆) δ 10.22 (s, 1H), 9.97 (s, 1H), 7.67 (dd, J = 8.0, 1.6 Hz, 1H), 7.47 − 7.35 (m, 2H), 6.87 (d, J = 2.4 Hz, 1H), 6.83 (dd, J = 8.4, 2.4 Hz, 1H), 6.69 (d, J = 8.4 Hz, 1H), 2.77 (dd, J = 8.8, 6.8 Hz, 2H), 2.47 (d, J = 2.0 Hz, 3H), 2.38 (dd, J = 8.4, 6.8 Hz, 2H). | 335.1 |
| Example 9 Method 1 | 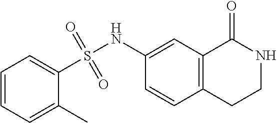 [SMILES: Cc1ccccc1S(=O)(=O)Nc1ccc2c(c1)C(=O)NCC2] 2-methyl-N-(1-oxo-1,2,3,4- tetrahydroisoquinolin-7- yl)benzenesulfonamide | White solid, yield: 6.49% | ¹H NMR (400 MHz, DMSO-d₆) δ 10.46 (s, 1H), 7.89 (s, 1H), 7.85 (d, J = 8.4 Hz, 1H), 7.57 (d, J = 2.0 Hz, 1H), 7.51 (td, J = 7.6, 1.2 Hz, 1H), 7.37 − 7.33 (m, 2H), 7.19 − 7.13 (m, 2H), 3.28 (td, J = 6.4, 2.8 Hz, 2H), 2.75 (t, J = 6.4 Hz, 2H), 2.59 (s, 3H). | 316.3 |
| Example 10 Method 1 | 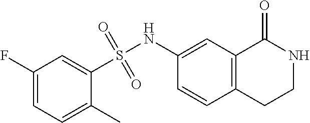 [SMILES: Cc1ccc(F)cc1S(=O)(=O)Nc1ccc2c(c1)C(=O)NCC2] 5-fluoro-2-methyl-N-(1-oxo-1,2,3,4- tetrahydroisoquinolin-7- | White solid, yield: 6.1% | ¹H NMR (400 MHz, DMSO-d₆) δ 10.59 (s, 1H), 7.91 (t, J = 2.8 Hz, 1H), 7.61 (dd, J = 2.8, 8.8 Hz, 1H), 7.56 (d, J = 1.6 Hz, 1H), 7.45 − 7.36 (m, 1H), 7.21 − 7.16 (m, 1H), 3.30 (td, J = 2.8, 6.4 Hz, 2H), 2.77 (t, J = 6.8 Hz, 2H), 2.54 (s, 3H). | 334.3 |
| | yl)benzenesulfonamide | | | |
| Example 11 Method 1 | 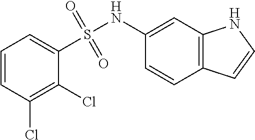 [SMILES: O=S(=O)(Nc1ccc2cc[nH]c2c1)c1cccc(Cl)c1Cl] 2,3-dichloro-N-(1H-indol-6- yl)benzenesulfonamide | white solid, yield: 23.2% | ¹H NMR (400 MHz, DMSO-d₆) δ 11.01 (s, 1H), 10.44 (s, 1H), 7.91 (dd, J = 8.0, 1.5 Hz, 1H), 7.85 (dd, J = 8.0, 1.6 Hz, 1H), 7.45 (t, J = 8.0 Hz, 1H), 7.37 (d, J = 8.4 Hz, 1H), 7.25 (t, J = 2.8 Hz, 1H), 7.20 − 7.14 (m, 1H), 6.80 (dd, J = 8.4, 2.0 Hz, 1H), 6.31 − 6.30 (m, 1H). | 340.0 |
| Example 12 Method 1 | 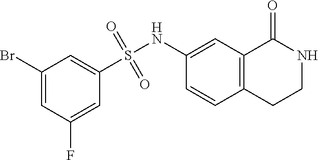 [SMILES: O=C1NCCc2ccc(NS(=O)(=O)c3cc(F)cc(Br)c3)cc21] | White solid, yield: 8.2% | ¹H NMR (400 MHz, DMSO-d₆) δ 10.54 (s, 1H), 7.93 (t, J = 2.8 Hz, 1H), 7.90 (td, J = 2.0, 8.4 Hz, 1H), 7.71 (t, J = 1.6 Hz, 1H), 7.56 − 7.53 (m, 2H), 7.21 (d, J = 1.6 Hz, 2H), 3.29 (m, 2H), 2.80 (t, J = 6.8 Hz, 2H). | 399.2 |
| | 3-bromo-5-fluoro-N- | | | |
| | (1-oxo-1,2,3,4- | | | |
| | tetrahydroisoquinolin-7- | | | |
| | yl)benzenesulfonamide | | | |
| Example 13 Method 1 | 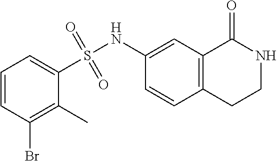 [SMILES: Cc1c(Br)cccc1S(=O)(=O)Nc1ccc2c(c1)C(=O)NCC2] | White solid, yield: 10.9% | ¹H NMR (400 MHz, DMSO-d₆) δ 10.67 (s, 1H), 7.91 − 7.86 (m, 3H), 7.57 (t, J = 1.6 Hz, 1H), 7.31 (t, J = 8.0 Hz, 1H), 7.17 (d, J = 1.6 Hz, 2H), 3.30 (dt, J = 2.8, 6.4 Hz, 2H), 2.77 (t, J = 6.4 Hz, 2H), 2.67 (s, 3H). | 396.1 |
| | 3-bromo-2-methyl- | | | |
| | N-(1-oxo-1,2,3,4- | | | |
| | tetrahydroisoquinolin-7- | | | |
| | yl)benzenesulfonamide | | | |
| Example 14 Method 1 | 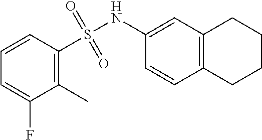 [SMILES: Cc1c(F)cccc1S(=O)(=O)Nc1ccc2c(c1)CCCC2] 3-fluoro-2-methyl-N-(5,6,7,8- | White solid, yield: 36.5% | ¹H NMR (400 MHz, DMSO-d₆) δ 10.30 (s, 1H), 7.71 (dd, J = 7.6, 1.6 Hz, 1H), 7.46 − 7.36 (m, 2H), 6.89 (d, J = 8.0 Hz, 1H), 6.79 − 6.74 (m, 2H), 2.57 (p, J = 3.2 Hz, 4H), 2.47 (d, J = 2.4 Hz, 3H), 1.64 (p, J = 3.2 Hz, 4H). | 320.1 |
| | tetrahydronaphthalen-2- | | | |
| | yl)benzenesulfonamide | | | |
| Example 15 Method 1 | 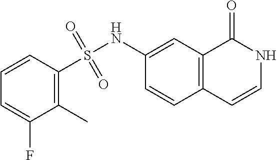 [SMILES: Cc1c(F)cccc1S(=O)(=O)Nc1ccc2cc[nH]c(=O)c2c1] | White solid, yield: 48.2% | ¹H NMR (400 MHz, Chloroform-d) δ 9.72 (s, 1H), 8.22-8.06 (m, 2H), 7.84 (d, J = 7.2 Hz, 1H), 7.68 (d, J = 8.4 Hz, 1H), 7.52 − 7.48 (m, 1H), 7.32 − 7.17 (m, 2H), 7.07 (s, 1H), 6.54 (s, 1H), 2.57 (d, J = 2.0 Hz, 3H). | 333.2 |
| | 3-fluoro-2-methyl-N- | | | |
| | (1-oxo-1,2- | | | |
| | dihydroisoquinolin-7- | | | |
| | yl)benzenesulfonamide | | | |
| Example 16 Method 1 | 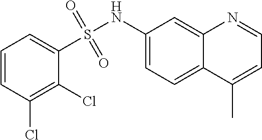 [SMILES: Cc1ccnc2cc(NS(=O)(=O)c3cccc(Cl)c3Cl)ccc12] 2,3-dichloro-N-(4-methylquinolin-7- yl)benzenesulfonamide | White solid, yield: 42.3% | ¹H NMR (400 MHz, DMSO-d₆) δ 11.29 (s, 1H), 8.64 (d, J = 4.4 Hz, 1H), 8.16 (dd, J = 8.0, 1.6 Hz, 1H), 8.01 (d, J = 9.0 Hz, 1H), 7.91 (dd, J = 8.2, 1.6 Hz, 1H), 7.62 (d, J = 2.4 Hz, 1H), 7.58 (t, J = 8.0 Hz, 1H), 7.43 (dd, J = 9.0, 2.4 Hz, 1H), 7.27 − 7.19 (m, 1H), 2.59 (s, 3H). | 267.1 |
| Example 17 Method 1 | 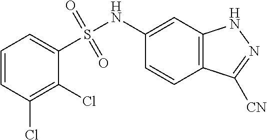 [SMILES: N#Cc1n[nH]c2cc(NS(=O)(=O)c3cccc(Cl)c3Cl)ccc12] | White solid, yield: 17.1% | ¹H NMR (400 MHz, Methanol-d₄) δ 8.02 − 8.00 (m, 1H), 7.68 − 7.59 (m, 2H), 7.39 − 7.31 (m, 2H), 7.1 − 7.08 (s, 1H). | 368.9 |
| | 6-nitro-1H-indazole- | | | |
| | 3-carbonitrile | | | |
| Example 18 Method 1 | 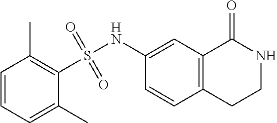 [SMILES: Cc1cccc(C)c1S(=O)(=O)Nc1ccc2c(c1)C(=O)NCC2] 2,6-dimethyl-N-(1-oxo-1,2,3,4- tetrahydroisoquinolin-7- yl)benzenesulfonamide | White solid, yield: 24.2% | ¹H NMR (400 MHz, DMSO-d₆) δ 10.30 (s, 1H), 7.86 (t, J = 2.8 Hz, 1H), 7.53 (d, J = 2.4 Hz, 1H), 7.31 (t, J = 8.0 Hz, 1H), 7.18 − 7.07 (m, 3H), 7.09 (dd, J = 8.0, 2.4 Hz, 1H), 3.3 (td, J = 6.4, 2.8 Hz, 2H), 2.74 (t, J = 6.4 Hz, 2H), 2.60 (s, 6H). | 331.2 |
| Example 19 Method 1 | 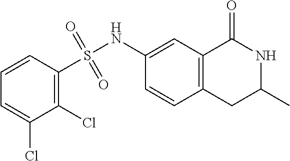 [SMILES: CC1Cc2ccc(NS(=O)(=O)c3cccc(Cl)c3Cl)cc2C(=O)N1] | White solid, yield: 18.8% | ¹H NMR (400 MHz, DMSO-d₆) δ 10.87 (s, 1H), 8.05 − 7.98 (m, 1H), 7.95 − 7.89 (m, 2H), 7.63 − 7.50 (m, 2H), 7.18 (d, J = 9.4 Hz, 1H), 3.31 − 3.27 (m, 1H), 3.17 (d, J = 5.2 Hz, 3H), 1.13 (d, J = 6.4 Hz, 3H). | 384.98 |
| | 2,3-dichloro-N-(3- | | | |
| | methyl-1-oxo- | | | |
| | 1,2,3,4- | | | |
| | tetrahydroisoquinolin-7- | | | |
| | yl)benzenesulfonamide | | | |
| Example 20 Method 1 | 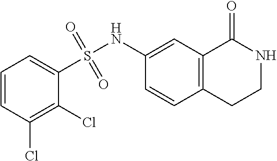 [SMILES: O=C1NCCc2ccc(NS(=O)(=O)c3cccc(Cl)c3Cl)cc21] | White solid, yield: 46.0% | ¹H NMR (400 MHz, DMSO-d₆) δ 10.84 (s, 1H), 8.02 (dd, J = 8.0, 1.6 Hz, 1H), 7.93 − 7.91 (m, 2H), 7.61 (d, J = 2.0 Hz, 1H), 7.54 (t, J = 8.0 Hz, 1H), 7.22 − 7.17 (m, 2H), 3.31 (dd, J = 6.4, 2.8 Hz, 2H), 2.77 (t, J = 6.4 Hz, 2H). | 371.1 |
| | 2,3-dichloro-N-(1- | | | |
| | oxo-1,2,3,4- | | | |
| | tetrahydroisoquinolin-7- | | | |
| | yl)benzenesulfonamide | | | |
| Example 21 Method 1 | 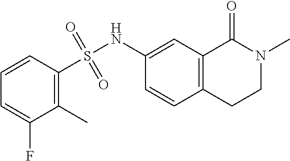 [SMILES: Cc1c(F)cccc1S(=O)(=O)Nc1ccc2c(c1)C(=O)N(C)CC2] | White solid, yield: 10.9% | ¹H NMR (400 MHz, DMSO-d₆) δ 10.78 (s, 1H), 7.71 (dd, J = 1.6, 7.6 Hz, 1H) 7.56 (d, J = 2.4 Hz, 1H), 7.43 − 7.34 (m, 2H), 7.14 − 7.09 (m, 2H), 3.45 (t, J = 6.8 Hz, 2H), 2.97 (s, 3H), 2.83 (t, J = 6.4 Hz, 2H), 2.48 (d, J = 2.4 Hz, 3H). | 349.1 |
| | 3-fluoro-2-methyl-N- | | | |
| | (2-methyl-1-oxo- | | | |
| | 1,2,3,4- | | | |
| | tetrahydroisoquinolin-7- | | | |
| | yl)benzenesulfonamide | | | |
| Example 22 Method 1 | 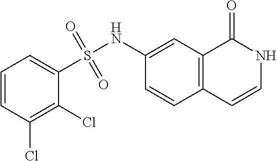 [SMILES: O=c1[nH]ccc2ccc(NS(=O)(=O)c3cccc(Cl)c3Cl)cc12] | White solid, yield: 37.3% | ¹H NMR (400 MHz, DMSO-d₆) δ 11.19 (d, J = 5.6 Hz, 1H), 11.12 (s, 1H), 8.05 (dd, J = 8.0, 1.6 Hz, 1H), 7.89 − 7.87 (m, 2H), 7.53 (t, J = 8.0 Hz, 2H), 7.46 (dd, J = 8.8, 2.4 Hz, 1H), 7.06 (dd, J = 5.6, 6.8 Hz, 1H), 6.43 (d, J = 6.8 Hz, 1H). | 368.8 |
| | 2,3-dichloro-N-(1- | | | |
| | oxo-1,2- | | | |
| | dihydroisoquinolin-7- | | | |
| | yl)benzenesulfonamide | | | |
| Example 23 Method 1 | 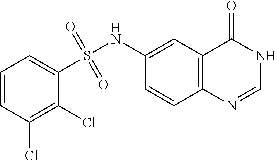 [SMILES: O=c1[nH]cnc2ccc(NS(=O)(=O)c3cccc(Cl)c3Cl)cc12] | White solid, yield: 1.4% | ¹H NMR (400 MHz, DMSO-d₆) δ 11.74 (s, 1H), 8.30 (s, 1H), 7.93 (dd, J = 7.6, 1.6 Hz, 1H), 7.74 (s, 1H), 7.64 (d, J = 8.0 Hz, 1H), 7.50 (d, J = 2.4 Hz, 1H), 7.37 − 7.24 (m, 3H). | 370.1 |
| | 2,3-dichloro-N-(4- | | | |
| | oxo-3,4- | | | |
| | dihydroquinazolin-6- | | | |
| | yl)benzenesulfonamide | | | |
| Example 24 Method 1 | 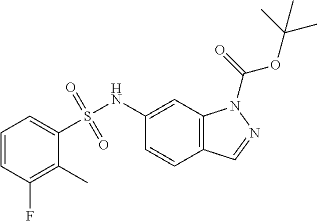 [SMILES: Cc1c(F)cccc1S(=O)(=O)Nc1ccc2cnn(C(=O)OC(C)(C)C)c2c1] | White solid, yield: 12.3% | ¹H NMR (400 MHz, DMSO-d₆) δ 10.99 (s, 1H), 8.27 (s, 1H), 7.87 − 7.82 (s, 1H), 7.75 (m, 2H), 7.42 − 7.37 (m, 2H), 7.14 (dd, J = 8.8, 2.0 Hz, 1H), 2.49 (m, 3H), 1.62 (s, 9H). | 406.2 |
| | tert-butyl 6-((3- | | | |
| | fluoro-2- | | | |
| | methylphenyl) | | | |
| | sulfonamido)-1H-indazole- | | | |
| | 1-carboxylate | | | |
| Example 25 Method 1 | 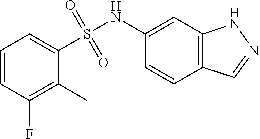 [SMILES: Cc1c(F)cccc1S(=O)(=O)Nc1ccc2cn[nH]c2c1] 3-fluoro-N-(1H-indazol-6-yl)-2- methylbenzenesulfonamide | white solid, yield: 33.3% | ¹H NMR (400 MHz, DMSO-d₆) δ 12.83 (s, 1H), 10.65 (s, 1H), 7.94 (d, J = 1.2 Hz, 1H), 7.73 (dd, J = 7.6, 1.6 Hz, 1H), 7.62 (d, J = 8.4 Hz, 1H), 7.44 − 7.37 (m, 2H), 7.25 (d, J = 1.2 Hz, 1H), 6.89 (dd, J = 8.4, 1.6 Hz, 1H), 2.49 (m, 3H). | 306.2 |
| Example 26 Method 1 | 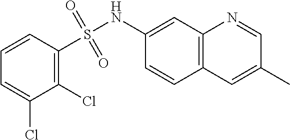 [SMILES: Cc1cnc2cc(NS(=O)(=O)c3cccc(Cl)c3Cl)ccc2c1] 2,3-dichloro-N-(3- | Light yellow solid, yield: 38% | ¹H NMR (400 MHz, DMSO-d₆) δ 8.65 (d, J = 2.2 Hz, 1H), 8.14 (dd, J = 7.9, 1.5 Hz, 1H), 7.98 (s, 1H), 7.90 (dd, J = 8.1, 1.5 Hz, 1H), 7.77 (d, J = 8.9 Hz, 1H), 7.66 − 7.53 (m, 2H), 7.36 (dd, J = 8.8, 2.2 Hz, 1H), 2.40 (s, 3H). | 376.02 |
| | methylquinolin-7- | | | |
| | yl)benzenesulfonamide | | | |
| Example 27 Method 1 | 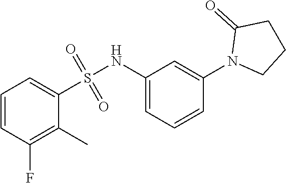 [SMILES: Cc1c(F)cccc1S(=O)(=O)Nc1cccc(N2CCCC2=O)c1] | orange solid, yield: 5.0% | ¹H NMR (400 MHz, DMSO-d₆) δ 10.57 (br s, 1H), 7.76 (d, J = 7.2 Hz, 1H), 7.56 (s, 1H), 7.44-7.38 (m, 2H), 7.18 (d, J = 5.2 Hz, 2H), 6.82-6.79 (m, 1H), 3.70 (t, J = 7.2 Hz, 2H), 2.47-2.44 (m, 5H), 2.05-1.99 (m, 2H). | 349.0 |
| | 3-fluoro-2-methyl-N- | | | |
| | (3-(2-oxopyrrolidin-1- | | | |
| | yl)phenyl)benzenesulfonamide | | | |
| Example 28 Method 1 | 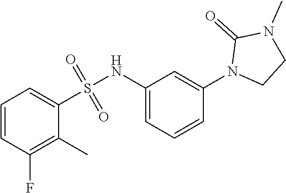 [SMILES: Cc1c(F)cccc1S(=O)(=O)Nc1cccc(N2CCN(C)C2=O)c1] | Light yellow solid, yield: 33.4% | ¹H NMR (400 MHz, DMSO-d₆) δ 10.49 (s, 1H), 7.76 (d, J =7.2 Hz, 1H), 7.48-7.40 (m, 3H), 7.13-7.11 (m, 2H), 6.69-6.67 (m, 1H), 3.67-3.63 (m, 2H), 3.41-3.37 (m, 2H), 2.74 (s, 3H), 2.51 (s, 3H). | 364.0 |
| | 3-fluoro-2-methyl-N- | | | |
| | (3-(3-methyl-2- | | | |
| | oxoimidazolidin-1- | | | |
| | yl)phenyl)benzenesulfonamide | | | |
| Example 29 Method 1 | 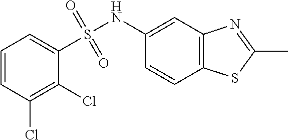 [SMILES: Cc1nc2cc(NS(=O)(=O)c3cccc(Cl)c3Cl)ccc2s1] | White solid, yield: 32.2% | ¹H NMR (400 MHz, DMSO-d₆) δ 10.98 (s, 1H), 8.06 (dd, J = 8.0, 1.6 Hz, 1H), 7.94 − 7.84 (m, 2H), 7.58 (d, J = 2.0 Hz, 1H), 7.53 (t, J = 8.0 Hz, 1H), 7.16 (dd, J = 8.6, 2.2 Hz, 1H), 2.73 (s, 3H). | 373.2 |
| | 2,3-dichloro-N-(2- | | | |
| | methylbenzo[d]thiazol-5- | | | |
| | yl)benzenesulfonamide | | | |
| Example 30 Method 1 | 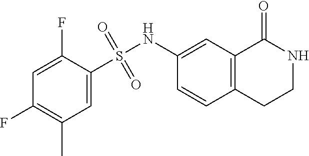 [SMILES: Cc1cc(S(=O)(=O)Nc2ccc3c(c2)C(=O)NCC3)c(F)cc1F] | White solid, yield: 16.6% | ¹H NMR (400 MHz, DMSO-d₆) δ 10.63 (s, 1H), 7.90 (s, 1H), 7.77 (t, J = 8.4 Hz, 1H), 7.59 (d, J = 2.4 Hz, 1H), 7.45 (t, J = 10.0 Hz, 1H), 7.24 − 7.16 (m, 2H), 3.29 − 3.26 (m, 2H), 2.78 (t, J = 6.6 Hz, 2H), 2.22 (s, 3H). | 353.3 |
| | 2,4-difluoro-5- | | | |
| | methyl-N-(1-oxo- | | | |
| | 1,2,3,4- | | | |
| | tetrahydroisoquinolin-7- | | | |
| | yl)benzenesulfonamide | | | |
| Example 31 Method 1 | 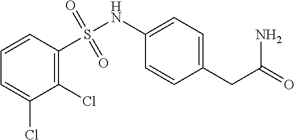 [SMILES: NC(=O)Cc1ccc(NS(=O)(=O)c2cccc(Cl)c2Cl)cc1] 2-(4-((2,3- | Light yellow solid, yield: 28.4% | ¹H NMR (400 MHz, DMSO-d₆) δ 10.66 (s, 1H), 8.00 (dd, J = 8.0, 1.6 Hz, 1H), 7.90 (d, J = 8.2 Hz, 1H), 7.52 (d, J = 8.0 Hz, 1H), 7.36 (br s, 1H), 7.10 (d, J = 8.6 Hz, 2H), 7.02 (d, J = 8.6 Hz, 2H), 6.79 (br s, 1H), 3.24 (s, 2H). | 359.3 |
| | dichlorophenyl) | | | |
| | sulfonamido)phenyl)acetamide | | | |
| Example 32 Method 1 | 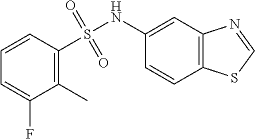 [SMILES: Cc1c(F)cccc1S(=O)(=O)Nc1ccc2scnc2c1] N-(benzo[d]thiazol-5-yl)-3-fluoro-2- | White solid, yield: 20.3% | ¹H NMR (400 MHz, DMSO-d₆) δ 10.80 (br s, 1H), 9.35 (s, 1H), 8.02 (d, J = 8.6 Hz, 1H), 7.77 (dd, J = 7.4, 1.6 Hz, 1H), 7.72 (d, J = 2.0 Hz, 1H), 7.48 − 7.35 (m, 2H), 7.23 (dd, J = 8.7, 2.2 Hz, 1H), 3.32 (s, 3H). | 323.2 |
| | methylbenzenesulfonamide | | | |
| Example 33 Method 1 | 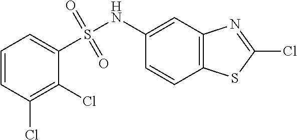 [SMILES: O=S(=O)(Nc1ccc2sc(Cl)nc2c1)c1cccc(Cl)c1Cl] | White solid, yield: 26.3% | ¹H NMR (400 MHz, DMSO-d₆) δ 11.13 (s, 1H), 8.09 (dd, J = 8.0, 1.6 Hz, 1H), 7.96 (d, J = 8.8 Hz, 1H), 7.91 (dd, J = 8.1, 1.5 Hz, 1H), 7.61 (d, J = 2.1 Hz, 1H), 7.54 (t, J = 8.0 Hz, 1H), 7.25 (dd, J = 8.8, 2.2 Hz, 1H). | 393.1 |
| | 2,3-dichloro-N-(2- | | | |
| | chlorobenzo[d]thiazol-5- | | | |
| | yl)benzenesulfonamide | | | |
| Example 34 Method 1 | 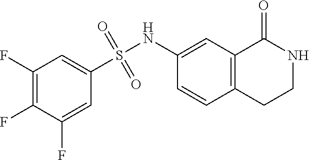 [SMILES: O=C1NCCc2ccc(NS(=O)(=O)c3cc(F)c(F)c(F)c3)cc21] | White solid, yield: 43.7% | ¹H NMR (400 MHz, DMSO-d₆) δ 10.58 (br s, 1H), 7.96 (s, 1H), 7.69 (t, J = 6.4 Hz, 2H), 7.58 (d, J = 2.0 Hz, 1H), 7.29 − 7.08 (m, 2H), 3.30 (td, J = 6.4, 2.8 Hz, 3H), 2.80 (t, J = 6.4 Hz, 2H). | 357.3 |
| | 3,4,5-trifluoro-N-(1- | | | |
| | oxo-1,2,3,4- | | | |
| | tetrahydroisoquinolin-7- | | | |
| | yl)benzenesulfonamide | | | |
| Example 35 Method 1 | 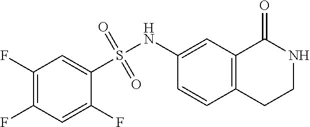 [SMILES: O=C1NCCc2ccc(NS(=O)(=O)c3cc(F)c(F)cc3F)cc21] | White solid, yield: 45.1% | ¹H NMR (400 MHz, DMSO-d₆) δ 10.85 (s, 1H), 7.95 (s, 1H), 7.92 − 7.80 (m, 2H), 7.59 (d, J = 2.0 Hz, 1H), 7.29 − 7.16 (m, 2H), 3.30 (td, J = 6.4, 2.8 Hz, 2H), 2.79 (t, J = 6.4 Hz, 2H). | 357.3 |
| | 2,4,5-trifluoro-N-(1- | | | |
| | oxo-1,2,3,4- | | | |
| | tetrahydroisoquinolin-7- | | | |
| | yl)benzenesulfonamide | | | |
| Example 36 Method 1 | 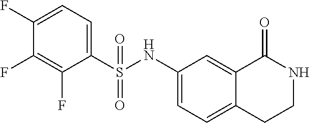 [SMILES: O=C1NCCc2ccc(NS(=O)(=O)c3ccc(F)c(F)c3F)cc21] 2,3,4-trifluoro-N-(1- | White solid, yield: 55.0% | ¹H NMR (400 MHz, DMSO-d₆) δ 7.93 (d, J = 2.8 Hz, 1H), 7.72 − 7.61 (m, 1H), 7.58 (d, J = 2.0 Hz, 1H), 7.53 − 7.41 (m, 1H), 7.24 − 7.15 (m, 2H), 3.32 − 3.27 (m, 2H), 2.79 (t, J = 6.4 Hz, 2H). | 357.1 |
| | oxo-1,2,3,4- | | | |
| | tetrahydroisoquinolin-7- | | | |
| | yl)benzenesulfonamide | | | |
| Example 37 Method 1 | 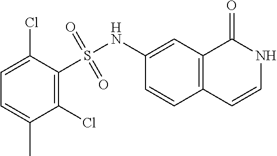 [SMILES: Cc1ccc(Cl)c(S(=O)(=O)Nc2ccc3cc[nH]c(=O)c3c2)c1Cl] | white solid, yield: 20.6% | ¹H NMR (400 MHz, DMSO-d₆) δ 11.17 (s, 1H), 11.05 (s, 1H), 7.89 (d, J = 2.1 Hz, 1H), 7.54 (t, J = 8.7 Hz, 3H), 7.49 − 7.44 (m, 1H), 7.04 (t, 1H), 6.42 (d, J = 7.0 Hz, 1H), 2.33 (s, 3H). | 383.25 |
| | 2,6-dichloro-3- | | | |
| | methyl-N-(1-oxo-2H- | | | |
| | isoquinolin-7- | | | |
| | yl)benzenesulfonamide | | | |
| Example 38 Method 1 | 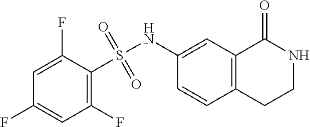 [SMILES: O=C1NCCc2ccc(NS(=O)(=O)c3c(F)cc(F)cc3F)cc21] 2,4,6-trifluoro-N-(1- | White solid, yield: 13.4% | ¹H NMR (400 MHz, DMSO-d₆) δ 10.99 (s, 1H), 7.95 (t, J = 2.8 Hz, 1H), 7.65 − 7.62 (m, 1H), 7.46 − 7.39 (m, 2H), 7.23 (d, J = 2.1 Hz, 2H), 3.32 − 3.27 (m, 2H), 2.80 (t, J = 6.6 Hz, 2H). | 357.0 |
| | oxo-3,4-dihydro-2H- | | | |
| | isoquinolin-7- | | | |
| | yl)benzenesulfonamide | | | |
| Example 39 Method 1 | 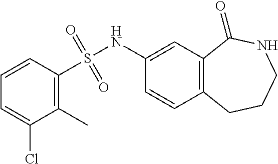 [SMILES: Cc1c(Cl)cccc1S(=O)(=O)Nc1ccc2c(c1)C(=O)NCCC2] 3-chloro-2-methyl-N- (1-oxo-2,3,4,5- | White solid, yield: 23.1% | ¹H NMR (400 MHz, DMSO-d₆) δ 10.67 (s, 1H), 8.03 (t, J = 6.0 Hz, 1H), 7.86 (dd, J = 8.0, 1.2 Hz, 1H), 7.71 (dd, J = 8.0, 1.4 Hz, 1H), 7.39 (t, J = 8.0 Hz, 1H), 7.22 − 7.17 (m, 1H), 7.14 − 7.05 (m, 2H), 2.81 (q, J = 6.4 Hz, 2H), 2.65 − 2.56 (m, 5H), 1.79 (p, J = 6.8 Hz, 2H). | 365.1 |
| | tetrahydro-1H- | | | |
| | benzo[c]azepin-8- | | | |
| | yl)benzenesulfonamide | | | |
| Example 40 Method 1 | 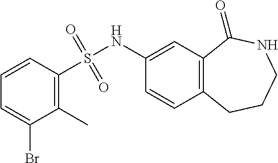 [SMILES: Cc1c(Br)cccc1S(=O)(=O)Nc1ccc2c(c1)C(=O)NCCC2] 3-bromo-2-methyl- N-(1-oxo-2,3,4,5- | White solid, yield: 20.1% | ¹H NMR (400 MHz, DMSO-d₆) δ 10.68 (br s, 1H), 8.03 (t, J = 6.0 Hz, 1H), 7.89 (ddd, J = 10.0, 8.0, 1.2 Hz, 2H), 7.31 (t, J = 8.0 Hz, 1H), 7.20 (d, J = 2.0 Hz, 1H), 7.15 − 7.05 (m, 2H), 2.81 (q, J = 6.4 Hz, 2H), 2.66 (s, 3H), 2.61 (t, J = 7.0 Hz, 2H), 1.79 (p, J = 6.8 Hz, 2H). | 409.2 |
| | tetrahydro-1H- | | | |
| | benzo[c]azepin-8- | | | |
| | yl)benzenesulfonamide | | | |
| Example 41 Method 1 | 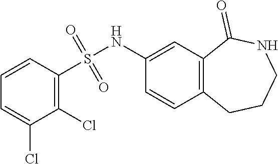 [SMILES: O=C1NCCCc2ccc(NS(=O)(=O)c3cccc(Cl)c3Cl)cc21] | White solid, yield: 21.1% | ¹H NMR (400 MHz, DMSO-d₆) δ 10.86 (s, 1H), 8.06 − 7.98 (m, 2H), 7.92 (dd, J = 8.0, 1.6 Hz, 1H), 7.55 (t, J = 8.0 Hz, 1H), 7.24 (t, J = 1.4 Hz, 1H), 7.15 − 7.09 (m, 2H), 2.80 (q, J = 6.4 Hz, 2H), 2.60 (t, J = 7.0 Hz, 2H), 1.79 (p, J = 6.8 Hz, 2H). | 385.1 |
| | 2,3-dichloro-N-(1- | | | |
| | oxo-2,3,4,5- | | | |
| | tetrahydro-1H- | | | |
| | benzo[c]azepin-8- | | | |
| | yl)benzenesulfonamide | | | |
| Example 42 Method 1 | 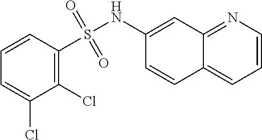 [SMILES: O=S(=O)(Nc1ccc2cccnc2c1)c1cccc(Cl)c1Cl] 2,3-dichloro-N-(quinolin-7- | White solid, yield: 17.8% | ¹H NMR (400 MHz, DMSO-d₆) δ 11.33 (br s, 1H), 8.79 (d, J = 2.6 Hz, 1H), 8.23 (dd, J = 8.2, 1.6 Hz, 1H), 8.17 (dd, J = 8.0, 1.6 Hz, 1H), 7.91 (dd, J = 8.2, 1.6 Hz, 1H), 7.88 (d, J = 8.8 Hz, 1H), 7.64 − 7.54 (m, 2H), 7.44 − 7.36 (m, 2H). | 353.1 |
| | yl)benzenesulfonamide | | | |
| Example 43 Method 1 | 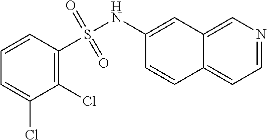 [SMILES: O=S(=O)(Nc1ccc2ccncc2c1)c1cccc(Cl)c1Cl] 2,3-dichloro-N- | White solid, yield: 22.3% | ¹H NMR (400 MHz, DMSO-d₆) δ 11.30 (br s, 1H), 9.19 (s, 1H), 8.39 (d, J = 5.6 Hz, 1H), 8.15 (dd, J = 8.0, 1.6 Hz, 1H), 7.93 − 7.84 (m, 2H), 7.73 (d, J = 2.2 Hz, 1H), 7.71 (d, J = 5.6 Hz, 1H), 7.60 − 7.49 (m, 2H). | 353.2 |
| | (isoquinolin-7- | | | |
| | yl)benzenesulfonamide | | | |
| Example 44 Method 1 | 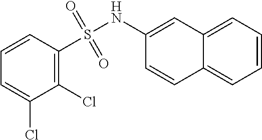 [SMILES: O=S(=O)(Nc1ccc2ccccc2c1)c1cccc(Cl)c1Cl] 2,3-dichloro-N-(naphthalen-2- yl)benzenesulfonamide | White solid, yield: 43.1% | ¹H NMR (400 MHz, DMSO-d₆) δ 11.03 (br s, 1H), 8.11 (dd, J = 8.0, 1.6 Hz, 1H), 7.88 (dd, J = 8.0, 1.4 Hz, 1H), 7.84 − 7.71 (m, 3H), 7.55 (d, J = 2.2 Hz, 1H), 7.52 (t, J = 8.0 Hz, 1H), 7.47- 7.36 (m, 2H), 7.31 (dd, J = 8.8, 2.2 Hz, 1H). | 352.1 |
| Example 45 Method 1 | 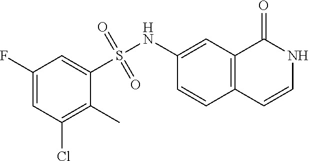 [SMILES: Cc1c(Cl)cc(F)cc1S(=O)(=O)Nc1ccc2cc[nH]c(=O)c2c1] 3-chloro-5-fluoro-2-methyl-N-(1-oxo- 1,2-dihydroisoquinolin- 7-yl)benzenesulfonamide | White solid, yield: 16.2% | ¹H NMR (400 MHz, DMSO-d₆) δ 11.24 (d, J = 5.8 Hz, 1H), 11.03 (s, 1H), 7.85 (d, J = 2.4 Hz, 1H), 7.80 (dd, J = 8.2, 2.8 Hz, 1H), 7.69 (dd, J = 8.4, 2.8 Hz, 1H), 7.58 (d, J = 8.6 Hz, 1H), 7.45 (dd, J = 8.6, 2.4 Hz, 1H), 7.07 (dd, J = 7.1, 5.8 Hz, 1H), 6.46 (d, J = 7.1 Hz, 1H), 2.60 (s, 3H). | 367.3 |
| Example 46 Method 1 | 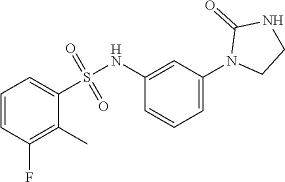 [SMILES: Cc1c(F)cccc1S(=O)(=O)Nc1cccc(N2CCNC2=O)c1] | White solid, yield: 3.0% | ¹H NMR (400 MHz, DMSO-d₆) δ 10.47 (t, J = 2.4 Hz, 1H), 7.75 (d, J = 7.6 Hz, 1H), 7.36- 7.44 (m, 3H), 7.07-7.13 (m, 2H), 6.94 (s, 1H), 6.88-6.68 (m, 1H), 3.71 (d, J = 8.0, 2H) 3.59 (d, J = 8.0, 2H) 2.32-2.47 (m, 3H). | 350.1 |
| | 3-fluoro-2-methyl-N- | | | |
| | (3-(2- | | | |
| | oxoimidazolidin-1- | | | |
| | yl)phenyl) | | | |
| | benzenesulfonamide | | | |
| Example 47 Method 1 | 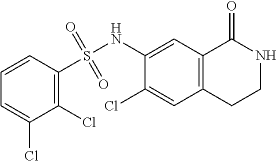 [SMILES: O=C1NCCc2cc(Cl)c(NS(=O)(=O)c3cccc(Cl)c3Cl)cc21] | light yellow solid. yield: 18.4% | ¹H NMR (400 MHz, DMSO-d₆) δ 8.65 (d, J = 2.2 Hz, 1H), 8.14 (dd, J = 7.9, 1.5 Hz, 1H), 7.98 (s, 1H), 7.90 (dd, J = 8.1, 1.5 Hz, 1H), 7.77 (d, J = 8.9 Hz, 1H), 7.66 − 7.50 (m, 2H), 7.36 (dd, J = 8.8, 2.2 Hz, 1H), 2.40 (s, 3H). | 404.97 |
| | 2,3-dichloro-N-(6- | | | |
| | chloro-1-oxo-1,2,3,4- | | | |
| | tetrahydroisoquinolin-7- | | | |
| | yl)benzenesulfonamide | | | |
| Example 48 Method 1 Mixture of Stereoisomers | 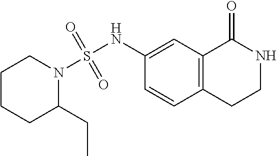 [SMILES: CCC1CCCCN1S(=O)(=O)Nc1ccc2c(c1)C(=O)NCC2] 2-ethyl-N-(1-oxo-1,2,3,4- tetrahydroisoquinolin- 7-yl)piperidine-1-sulfonamide | White solid, yield: 3.3% | ¹H NMR (400 MHz, DMSO-d₆) δ 9.86 (s, 1H), 7.88 (s, 1H), 7.60 (d, J = 2.0 Hz, 1H), 7.17 (dd, J = 9.4, 5.2 Hz, 2H), 3.65 (dd, J = 12.0, 7.2 Hz, 1H), 3.41 (dd, J = 13.8, 4.0 Hz, 1H), 3.27 (d, J = 2.8 Hz, 1H), 2.85 − 2.75 (m, 3H), 1.57 − 1.25 (m, 8H), 1.00 (m, 1H), 0.72 (t, J = 7.4 Hz, 3H). | 338.0 |
| Example 49 Method 1 | 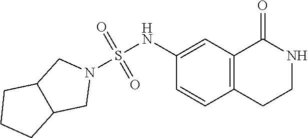 [SMILES: O=C1NCCc2ccc(NS(=O)(=O)N3CC4CCCC4C3)cc21] N-(1-oxo-3,4- | White solid, yield: 12.7% | ¹H NMR (400 MHz, DMSO-d₆) δ 7.69 (d, J = 2.3 Hz, 1H), 7.29 − 7.20 (m, 2H), 3.32 (m, 2H), 3.23 (m, 2H), 2.86 − 2.79 (m, 4H), 2.58 − 2.52 (m, 2H), 1.67 − 1.56 (m, 2H), 1.42 − 1.28 (m, 2H), 1.20 − 1.10 (m, 2H). | 336.0 |
| | dihydro-2H- | | | |
| | isoquinolin-7-yl)- | | | |
| | 3,3a,4,5,6,6a- | | | |
| | hexahydro-1H- | | | |
| | cyclopenta[c]pyrrole- | | | |
| | 2-sulfonamide | | | |
| Example 50 Method 1 | 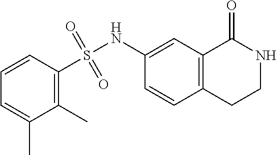 [SMILES: Cc1cccc(S(=O)(=O)Nc2ccc3c(c2)C(=O)NCC3)c1C] 2,3-dimethyl-N-(1-oxo-1,2,3,4- tetrahydroisoquinolin-7- | Light brown solid, yield: 45.8% | ¹H NMR (400 MHz, Methanol-d₄) δ 7.81 (dd, J = 8.0, 1.4 Hz, 1H), 7.61 (d, J = 2.4 Hz, 1H), 7.35 (d, J = 7.8 Hz, 1H), 7.22 (dd, J = 8.0, 2.4 Hz, 1H), 7.15 (t, J = 7.8 Hz, 1H), 7.11 (d, J = 8.0 Hz, 1H), 3.40 (t, J = 6.4 Hz, 2H), 2.84 (t, J = 6.4 Hz, 2H), 2.56 (s, 3H), 2.30 (s, 3H). | 331.1 |
| | yl)benzenesulfonamide | | | |
| Example 51 Method 1 | 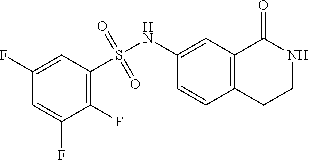 [SMILES: O=C1NCCc2ccc(NS(=O)(=O)c3cc(F)cc(F)c3F)cc21] | brown solid, yield: 17.9% | ¹H NMR (400 MHz, DMSO-d₆) δ 11.00 (s, 1H), 7.96 (d, J = 2.8 Hz, 1H), 7.95 − 7.86 (m, 1H), 7.60 (t, J = 1.4 Hz, 1H), 7.50 − 7.40 (m, 1H), 7.23 (d, J = 2.1 Hz, 2H), 3.30 (m, 2H), 2.80 (t, J = 6.6 Hz, 2H). | 357.0 |
| | 2,3,5-trifluoro-N-(1- | | | |
| | oxo-3,4-dihydro-2H- | | | |
| | isoquinolin-7- | | | |
| | yl)benzenesulfonamide | | | |
| Example 52 Method 1 | 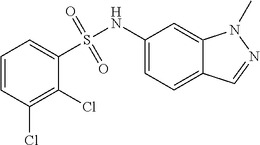 [SMILES: Cn1ncc2ccc(NS(=O)(=O)c3cccc(Cl)c3Cl)cc21] 2,3-dichloro-N-(1-methyl-1H-indazol- 6-yl)benzenesulfonamide | White solid, yield: 65.4% | ¹H NMR (400 MHz, DMSO-d₆) δ 10.99 (br s, 1H), 8.13 (dd, J = 8.0, 1.6 Hz, 1H), 7.92 (d, J = 1.0 Hz, 1H), 7.88 (dd, J = 8.0, 1.6 Hz, 1H), 7.63 (d, J = 8.4 Hz, 1H), 7.53 (t, J = 8.0 Hz, 1H), 7.24 (s, 1H), 6.93 (dd, J = 8.4, 2.0 Hz, 1H), 3.92 (s, 3H). | 356.2 |
| Example 53 Method 1 | 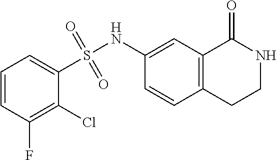 [SMILES: O=C1NCCc2ccc(NS(=O)(=O)c3cccc(F)c3Cl)cc21] 2-chloro-3-fluoro-N- | White solid, yield: 35.5% | ¹H NMR (400 MHz, DMSO-d₆) δ 10.85 (s, 1H), 7.93 (s, 1H), 7.86 (d, J = 6.4 Hz, 1H), 7.70 (td, J = 8.4, 1.6 Hz, 1H), 7.60 (d, J = 2.0 Hz, 1H), 7.59 − 7.52 (m, 1H), 7.24 − 7.10 (m, 2H), 3.28 (td, J = 6.4, 2.8 Hz, 2H), 2.77 (t, J = 6.4 Hz, 2H). | 355.2 |
| | (1-oxo-1,2,3,4- | | | |
| | tetrahydroisoquinolin-7- | | | |
| | yl)benzenesulfonamide | | | |
| Example 54 Method 1 | 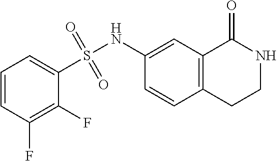 [SMILES: O=C1NCCc2ccc(NS(=O)(=O)c3cccc(F)c3F)cc21] | White solid, yield: 32.2% | ¹H NMR (400 MHz, DMSO-d₆) δ 10.87 (s, 1H), 7.95 (s, 1H), 7.78 − 7.71 (m, 1H), 7.67 − 7.53 (m, 2H), 7.41 − 7.35 (m, 1H), 7.27 − 7.14 (m, 2H), 3.29 (td, J = 6.7, 2.8 Hz, 2H), 2.79 (t, J = 6.6 Hz, 2H). | 339.2 |
| | 2,3-difluoro-N-(1- | | | |
| | oxo-1,2,3,4- | | | |
| | tetrahydroisoquinolin-7- | | | |
| | yl)benzenesulfonamide | | | |
| Example 55 Method 2 | 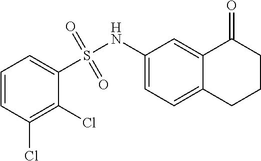 [SMILES: O=C1CCCc2ccc(NS(=O)(=O)c3cccc(Cl)c3Cl)cc21] 2,3-dichloro-N-(8-oxo-5,6,7,8- tetrahydronaphthalen-2- | White solid, yield: 40% | ¹H NMR (400 MHz, DMSO-d₆) δ 10.92 (s, 1H), 8.02 (dd, J = 8.0, 1.6 Hz, 1H), 7.92 (dd, J = 8.0, 1.6 Hz, 1H), 7.59 (d, J = 2.4 Hz, 1H), 7.55 (t, J = 8.0 Hz, 1H), 7.32 − 7.22 (m, 2H), 2.81 (t, J = 6.0 Hz, 2H), 2.53 (d, J = 5.6 Hz, 2H), 1.95 (p, J = 6.4 Hz, 2H). | 370.2 |
| | yl)benzenesulfonamide | | | |
| Example 56 Method 1 Mixture of Stereoisomers | 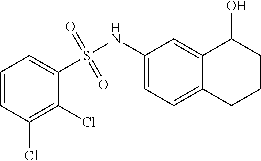 [SMILES: O=S(=O)(Nc1ccc2c(c1)C(O)CCC2)c1cccc(Cl)c1Cl] 2,3-dichloro-N-(8-hydroxy-5,6,7,8- tetrahydronaphthalen-2- yl)benzenesulfonamide | White solid, yield: 38.5% | ¹H NMR (400 MHz, DMSO-d₆) δ 10.59 (s, 1H), 8.00 (dd, J = 8.0, 1.5 Hz, 1H), 7.91 (dd, J = 8.1, 1.5 Hz, 1H), 7.53 (t, J = 8.0 Hz, 1H), 7.19 (d, J = 2.0 Hz, 1H), 6.95 − 6.86 (m, 2H), 5.10 (d, J = 6.2 Hz, 1H), 4.39 (s, 1H), 2.64 − 2.52 (m, 2H), 1.88 − 1.74 (m, 2H), 1.64 − 1.47 (m, 2H). | 372.1 |
| Example 57 Method 1 | 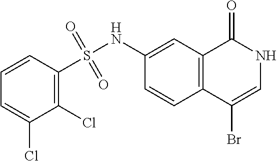 [SMILES: O=c1[nH]cc(Br)c2ccc(NS(=O)(=O)c3cccc(Cl)c3Cl)cc12] | White solid, yield: 3.5% | ¹H NMR (400 MHz, DMSO-d₆) δ 8.12 (dd, J = 8.0, 2.0 Hz, 2H), 7.85 (d, J = 9.0 Hz, 1H), 7.78 (s, 1H), 7.69 (d, J = 8.0 Hz, 1H), 7.40 (dd, J = 9.0, 2.2 Hz, 1H), 7.11 (d, J = 2.2 Hz, 1H), 6.39 (s, 1H). | 448.2 |
| | N-(4-bromo-1-oxo- | | | |
| | 1,2- | | | |
| | dihydroisoquinolin- | | | |
| | 7-yl)-2,3- | | | |
| | dichlorobenzenesulfonamide | | | |
| Example 58 Method 1 | 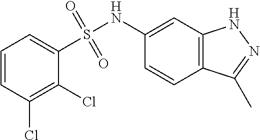 [SMILES: Cc1n[nH]c2cc(NS(=O)(=O)c3cccc(Cl)c3Cl)ccc12] 2,3-dichloro-N-(3-methyl-1H-indazol- 6-yl)benzenesulfonamide | white solid, yield: 52.1% | ¹H NMR (400 MHz, DMSO-d₆) δ 12.43 (s, 1H), 10.86 (br s, 1H), 8.01 (dd, J = 8.0, 1.6 Hz, 1H), 7.89 (dd, J = 8.0, 1.6 Hz, 1H), 7.55 (d, J = 8.4 Hz, 1H), 7.51 (t, J = 8.0 Hz, 1H).7.14 (d, J = 1.6 Hz, 1H), 6.88 (dd, J = 8.4, 1.6 Hz, 1H), 2.38 (s, 3H). | 356.1 |
| Example 59 Method 1 | 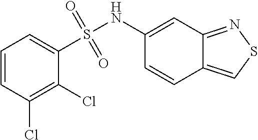 [SMILES: O=S(=O)(Nc1ccc2csnc2c1)c1cccc(Cl)c1Cl] N-(benzo[c]isothiazol-6-yl)-2,3- dichlorobenzenesulfonamide | white solid, yield: 54.2% | ¹H NMR (400 MHz, DMSO-d₆) δ 11.21 (s, 1H), 9.64 (d, J = 1.1 Hz, 1H), 8.16 (dd, J = 8.0, 1.5 Hz, 1H), 7.92 (dd, J = 8.1, 1.5 Hz, 1H), 7.80 (d, J = 9.2 Hz, 1H), 7.58 (t, J = 8.0 Hz, 1H), 7.32 − 7.27 (m, 1H), 7.13 (dd, J = 9.1, 2.0 Hz, 1H). | 359.0 |
| Example 60 Method 1 | 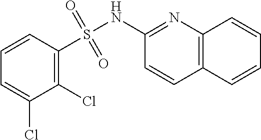 [SMILES: O=S(=O)(Nc1ccc2ccccc2n1)c1cccc(Cl)c1Cl] 2,3-dichloro-N-(quinolin-2- yl)benzenesulfonamide | white solid, yield: 33.7% | ¹H NMR (400 MHz, CDCl₃) δ 12.24 (s, 1H), 8.22 (dd, J = 8.0, 1.5 Hz, 1H), 7.96 (d, J = 9.4 Hz, 1H), 7.73 − 7.65 (m, 2H), 7.63 (dd, J = 8.0, 1.4 Hz, 1H), 7.50 (t, J = 7.5 Hz, 1H), 7.43 (t, J = 7.6 Hz, 1H), 7.36 (t, J = 8.0 Hz, 1H), 6.91 (d, J = 9.3 Hz, 1H). | 353.2 |
| Example 61 Method 1 | 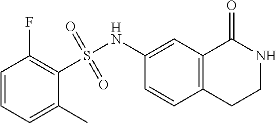 [SMILES: Cc1cccc(F)c1S(=O)(=O)Nc1ccc2c(c1)C(=O)NCC2] | White solid, yield: 54.1% | ¹H NMR (400 MHz, DMSO-d₆) δ 10.63 (s, 1H), 7.90 (s, 1H), 7.60 (s, 1H), 7.49 (td, J = 8.0, 5.5 Hz, 1H), 7.24 − 7.11 (m, 4H), 3.30 − 3.24 (m, 2H), 2.76 (t, J = 6.5 Hz, 2H), 2.59 (s, 3H). | 335.1 |
| | 2-fluoro-6-methyl-N- | | | |
| | (1-oxo-1,2,3,4- | | | |
| | tetrahydroisoquinolin-7- | | | |
| | yl)benzenesulfonamide | | | |
| Example 62 Method 1 | 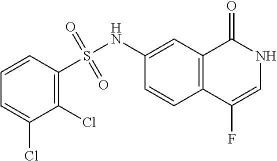 [SMILES: O=c1[nH]cc(F)c2ccc(NS(=O)(=O)c3cccc(Cl)c3Cl)cc12] | White solid, yield: 6.0% | ¹H NMR (400 MHz, DMSO-d₆) δ 8.65 (d, J = 2.2 Hz, 1H), 8.14 (dd, J = 7.9, 1.5 Hz, 1H), 7.98 (s, 1H), 7.90 (dd, J = 8.1, 1.5 Hz, 1H), 7.77 (d, J = 8.9 Hz, 1H), 7.66 − 7.50 (m, 2H), 7.36 (dd, J = 8.8, 2.2 Hz, 1H), 2.40 (s, 3H). | 387.1 |
| | 2,3-dichloro-N-(4- | | | |
| | fluoro-1-oxo-1,2- | | | |
| | dihydroisoquinolin-7- | | | |
| | yl)benzenesulfonamide | | | |
| Example 63 Method 1 | 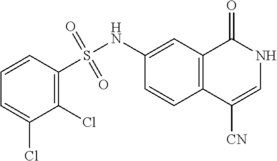 [SMILES: N#Cc1c[nH]c(=O)c2cc(NS(=O)(=O)c3cccc(Cl)c3Cl)ccc12] | Brown solid, yield: 2.2% | ¹H NMR (400 MHz, DMSO-d₆) δ 8.18 (d, J = 8.0 Hz, 1H), 8.09 (s, 1H), 7.85 (s, 1H), 7.79 − 7.69 (m, 3H), 7.46 (t, J = 8.0 Hz, 1H). | 391.8 |
| | 2,3-dichloro-N-(4- | | | |
| | cyano-1-oxo-1,2- | | | |
| | dihydroisoquinolin- | | | |
| | 7-yl)benzenesulfonamide | | | |
| Example 64 Method 1 | 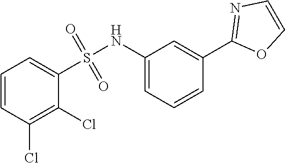 [SMILES: O=S(=O)(Nc1cccc(-c2ncco2)c1)c1cccc(Cl)c1Cl] 2,3-dichloro-N-(3-oxazol-2- ylphenyl)benzenesulfonamide | White solid, yield: 46.6% | ¹H NMR (400 MHz, DMSO-d₆) δ 11.09 (s, 1H), 8.21 (d, J = 0.8 Hz, 1H), 8.07 (dd, J = 8.0, 1.5 Hz, 1H), 7.93 (dd, J = 8.1, 1.5 Hz, 1H), 7.77 (t, J = 1.9 Hz, 1H), 7.66 − 7.60 (m, 1H), 7.56 (t, J = 8.0 Hz, 1H), 7.41 (t, J = 8.0 Hz, 1H), 7.37 (d, J = 0.8 Hz, 1H), 7.28 − 7.20 (m, 1H). | 369.2 |
| Example 65 Method 1 | 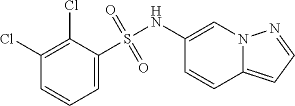 [SMILES: O=S(=O)(Nc1ccc2ccnn2c1)c1cccc(Cl)c1Cl] N-(1-(2,3- dichlorophenyl)ethyl) pyrazolo[1,5- a]pyridin-6-amine | White solid, Yield: 23.5% | ¹H NMR (400 MHz, DMSO-d₆) δ 10.80 (s, 1H), 8.35 (d, J = 1.6 Hz, 1H), 7.99 (dd, J = 8.0, 1.6 Hz, 1H), 7.93 (dd, J = 6.8, 2.0 Hz, 2H), 7.63 (d, J = 9.4 Hz, 1H), 7.52 (t, J = 8.0 Hz, 1H), 7.00 (dd, J = 9.4, 2.0 Hz, 1H), 6.57 (d, J = 2.4 Hz, 1H). | 342.1 |
| Example 66 Method 1 | 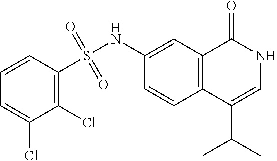 [SMILES: CC(C)c1c[nH]c(=O)c2cc(NS(=O)(=O)c3cccc(Cl)c3Cl)ccc12] | Yellow solid, Yield: 3.5% | ¹H NMR (400 MHz, DMSO-d₆) δ 11.00 (s, 1H), 8.04 − 8.00 (m, 1H), 7.89 − 7.76 (m, 2H), 7.64 (d, J = 8.8 Hz, 1H), 7.52 − 7.41 (m, 2H), 6.71 (d, J = 5.6 Hz, 1H), 3.15 − 3.09 (m, 1H), 1.17 (d, J = 6.8 Hz, 6H). | 410.9 |
| | 2,3-dichloro-N-(4- | | | |
| | isopropyl-1-oxo-1,2- | | | |
| | dihydroisoquinolin-7- | | | |
| | yl)benzenesulfonamide | | | |
| Example 67 Method 1 | 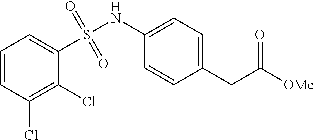 [SMILES: COC(=O)Cc1ccc(NS(=O)(=O)c2cccc(Cl)c2Cl)cc1] | | ¹H NMR (400 MHz, DMSO-d₆) δ 7.98 (dd, J = 8.0, 1.6 Hz, 1H), 7.86 (dd, J = 8.0, 1.6 Hz, 1H), 7.48 (t, J = 8.0 Hz, 1H), 7.06 (d, J = 8.6 Hz, 2H), 6.98 (d, J = 8.6 Hz, 2H), 3.53 (s, 3H), 3.51 (s, 2H). | 372.0 [M − H]⁻ |
| Example 68 Method 1 | 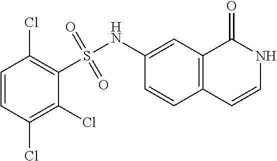 [SMILES: O=c1[nH]ccc2ccc(NS(=O)(=O)c3c(Cl)ccc(Cl)c3Cl)cc12] | White solid, yield: 12.55% | ¹H NMR (400 MHz, DMSO-d₆) δ 11.25 (s, 1H), 11.21 (s, 1H), 7.95 − 7.81 (m, 2H), 7.66 (d, J = 8.8 Hz, 1H), 7.59 (d, J = 8.7 Hz, 1H), 7.46 (dd, J = 8.7, 2.5 Hz, 1H), 7.07 (t, J = 6.4 Hz, 1H), 6.44 (d, J = 7.1 Hz, 1H). | 403.3 |
| | 2,3,6-trichloro-N-(1- | | | |
| | oxo-1,2- | | | |
| | dihydroisoquinolin- | | | |
| | 7- | | | |
| | yl)benzenesulfonamide | | | |
| Example 69 Method 1 | 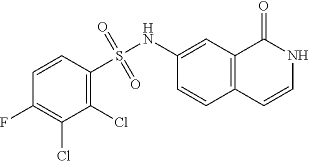 [SMILES: O=c1[nH]ccc2ccc(NS(=O)(=O)c3ccc(F)c(Cl)c3Cl)cc12] 2,3-dichloro-4- fluoro-N-(1-oxo-1,2- | White solid, yield: 37.78% | ¹H NMR (400 MHz, DMSO-d₆) δ 11.23 (d, J = 5.7 Hz, 1H), 11.13 (s, 1H), 8.10 (dd, J = 9.0, 5.6 Hz, 1H), 7.89 (d, J = 2.4 Hz, 1H), 7.63 (t, J = 8.6 Hz, 1H), 7.57 (d, J = 8.6 Hz, 1H), 7.46 (dd, J = 8.6, 2.4 Hz, 1H), 7.07 (dd, J = 7.1, 5.8 Hz, 1H), 6.44 (d, J = 7.1 Hz, 1H). | 387.2 |
| | dihydroisoquinolin- | | | |
| | 7- | | | |
| | yl)benzenesulfonamide | | | |
| Example 70 Method 1 | 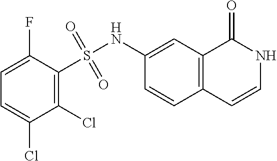 [SMILES: O=c1[nH]ccc2ccc(NS(=O)(=O)c3c(F)ccc(Cl)c3Cl)cc12] | White solid, yield: 10.42% | ¹H NMR (400 MHz, DMSO-d₆) δ 11.31 (s, 1H), 11.24 (d, J = 5.7 Hz, 1H), 8.02 − 7.89 (m, 2H), 7.60 (d, J = 8.6 Hz, 1H), 7.54 − 7.45 (m, 2H), 7.08 (dd, J = 7.1, 5.7 Hz, 1H), 6.46 (d, J = 7.1 Hz, 1H). | 387.2 |
| | 2,3-dichloro-6- | | | |
| | fluoro-N-(1-oxo-1,2- | | | |
| | dihydroisoquinolin- | | | |
| | 7- | | | |
| | yl)benzenesulfonamide | | | |
| Example 71 Method 1 | 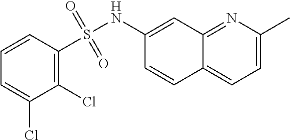 [SMILES: Cc1ccc2ccc(NS(=O)(=O)c3cccc(Cl)c3Cl)cc2n1] 2,3-dichloro-N-(2- methylquinolin-7- yl)benzenesulfonamide | | ¹H NMR (400 MHz, DMSO-d₆) δ 11.24 (s, 1H), 8.15 (dd, J = 8.0, 1.6 Hz, 1H), 8.10 (d, J = 8.4 Hz, 1H), 7.91 (dd, J = 8.0, 1.6 Hz, 1H), 7.80 (d, J = 8.8 Hz, 1H), 7.58 (t, J = 8.0 Hz, 1H), 7.52 (d, J = 2.2 Hz, 1H), 7.33 (dd, J = 8.8, 2.2 Hz, 1H), 7.28 (d, J = 8.4 Hz, 1H), 2.58 (s, 3H). | 367.1 |
| Example 72 Method 1 | 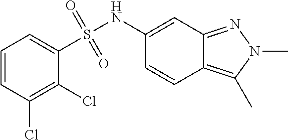 [SMILES: Cc1c2ccc(NS(=O)(=O)c3cccc(Cl)c3Cl)cc2nn1C] | | ¹H NMR (400 MHz, DMSO-d₆) δ 10.66 (s, 1H), 8.01 (dd, J = 8.0, 1.6 Hz, 1H), 7.87 (d, J = 8.0 Hz, 1H), 7.55 − 7.46 (m, 2H), 7.08 (s, 1H), 6.78 (d, J = 9.0 Hz, 1H), 3.93 (s, 3H) 3.33 (s, 3H). | 370.1 |
| | 2,3-dichloro-N-(2,3- | | | |
| | dimethyl-2H-indazol-6- | | | |
| | yl)benzenesulfonamide | | | |
| Example 73 Method 1 | 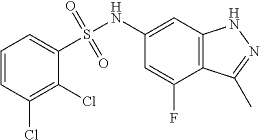 [SMILES: Cc1n[nH]c2cc(NS(=O)(=O)c3cccc(Cl)c3Cl)cc(F)c12] 2,3-dichloro-N-(4- | | ¹H NMR (400 MHz, DMSO-d₆) δ 12.71 (br, 1H), 11.08 (s, 1H), 8.06 (dd, J = 8.0, 1.5 Hz, 1H), 7.92 (dd, J = 8.1, 1.5 Hz, 1H), 7.56 (t, J = 8.1 Hz, 1H), 6.95 (d, J = 1.5 Hz, 1H), 6.63 (dd, J = 11.9, 1.5 Hz, 1H), 2.45 (s, 3H). | 373.1 |
| | fluoro-3-methyl-1H- | | | |
| | indazol-6- | | | |
| | yl)benzenesulfonamide | | | |
| Example 74 Method 1 | 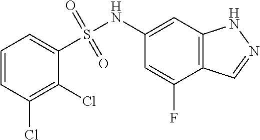 [SMILES: O=S(=O)(Nc1cc(F)c2cn[nH]c2c1)c1cccc(Cl)c1Cl] 2,3-dichloro-N-(4- | | ¹H NMR (400 MHz, DMSO-d₆) δ 13.18 (s, 1H), 11.13 (s, 1H), 8.07 (dd, J = 8.0, 1.6 Hz, 1H), 8.06-8.04 (m, 1H), 7.92 (dd, J = 8.0, 1.6 Hz, 1H), 7.56 (t, J = 8.0 Hz, 1H), 7.05 (t, J = 1.2 Hz, 1H), 6.71 (dd, J = 11.6, 1.6 Hz, 1H). | |
| | fluoro-1H-indazol-6- | | | |
| | yl)benzenesulfonamide | | | |
| Example 75 Method 1 | 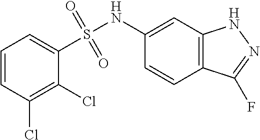 [SMILES: O=S(=O)(Nc1ccc2c(F)n[nH]c2c1)c1cccc(Cl)c1Cl] | White solid, yield: 18.5% | ¹H NMR (400 MHz, DMSO-d₆) δ 8.06 (d, J = 6.8 Hz, 1H), 7.78 − 7.69 (m, 1H), 7.49 (d, J = 8.6 Hz, 1H), 7.39 (t, J = 8.0 Hz, 1H), 7.22 (s, 1H), 6.95 (dd, J = 8.6, 1.2 Hz, 1H). | 359.9 |
| | 2,3-dichloro-N-(3- | | | |
| | fluoro-1H-indazol-6- | | | |
| | yl)benzenesulfonamide | | | |
| Example 76 Method 1 | 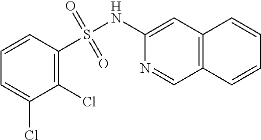 [SMILES: O=S(=O)(Nc1cc2ccccc2cn1)c1cccc(Cl)c1Cl] 2,3-dichloro-N- (isoquinolin-3- yl)benzenesulfonamide | Green solid, yield: 64.2% | ¹H NMR (400 MHz, DMSO-d₆) δ 11.75 (s, 1H), 8.99 (s, 1H), 8.19 (dd, J = 8.0, 1.4 Hz, 1H), 7.98 (d, J = 8.2 Hz, 1H), 7.90 (dd, J = 8.0, 1.3 Hz, 1H), 7.85 (d, J = 8.2 Hz, 1H), 7.69 (t, J = 7.6 Hz, 1H), 7.58 (t, J = 8.0 Hz, 1H), 7.50 (t, J = 7.6 Hz, 1H), 7.40 (s, 1H). | 352.9 |
| Example 77 Method 1 | 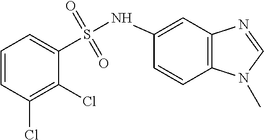 [SMILES: Cn1cnc2cc(NS(=O)(=O)c3cccc(Cl)c3Cl)ccc21] | white solid, yield: 14.8% | ¹H NMR (400 MHz, DMSO-d₆) δ 10.56 (s, 1H), 8.14 (s, 1H), 7.93 (dd, J = 8.0, 1.4 Hz, 1H), 7.87 (dd, J = 8.0, 1.4 Hz, 1H), 7.50 − 7.41 (m, 2H), 7.34 (s, 1H), 7.06 (dd, J = 8.6, 1.6 Hz, 1H), 3.34 (s, 3H). | 355.9 |
| | 2,3-dichloro-N-(1- | | | |
| | methyl-1H- | | | |
| | benzo[d]imidazol-5- | | | |
| | yl)benzenesulfonamide | | | |
| Example 78 Method 1 | 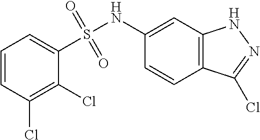 [SMILES: O=S(=O)(Nc1ccc2c(Cl)n[nH]c2c1)c1cccc(Cl)c1Cl] | white solid, yield: 17.2% | ¹H NMR (400 MHz, DMSO-d₆) δ 13.08 (s, 1H), 11.12 (s, 1H), 8.06 (dd, J = 8.0, 1.6 Hz, 1H), 7.92 (d, J = 8.0 Hz, 1H), 7.61 − 7.48 (m, 2H), 7.22 (d, J = 1.0 Hz, 1H), 7.03 (dd, J = 8.8, 1.6 Hz, 1H). | 375.9 |
| | 2,3-dichloro-N-(3- | | | |
| | chloro-1H-indazol-6- | | | |
| | yl)benzenesulfonamide | | | |
| Example 79 Method 1 | 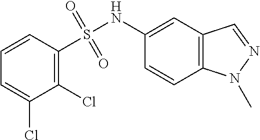 [SMILES: Cn1ncc2cc(NS(=O)(=O)c3cccc(Cl)c3Cl)ccc21] 2,3-dichloro-N-(1- | white solid, yield: 63.2% | ¹H NMR (400 MHz, DMSO-d₆) δ 10.59 (s, 1H), 7.97 (s, 1H), 7.92 (dd, J = 8.0, 1.2 Hz, 1H), 7.87 (dd, J = 8.0, 1.2 Hz, 1H), 7.53 (d, J = 8.8 Hz, 1H), 7.46 (dd, J = 9.4, 6.6 Hz, 2H), 7.17 (dd, J = 8.8, 2.0 Hz, 1H), 3.96 (s, 3H). | 356.0 |
| | methyl-1H-indazol- | | | |
| | 5- | | | |
| | yl)benzenesulfonamide | | | |
| Example 80 Method 1 | 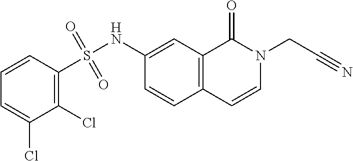 [SMILES: N#CCn1ccc2ccc(NS(=O)(=O)c3cccc(Cl)c3Cl)cc2c1=O] | White solid, yield: 4.0% | ¹H NMR (400 MHz, DMSO-d₆) δ 11.29 (s, 1H), 8.06 (dd, J = 8.0, 1.4 Hz, 1H), 7.92 − 7.86 (m, 2H), 7.59 − 7.47 (m, 3H), 7.40 (d, J = 7.4 Hz, 1H), 6.61 (d, J = 7.4 Hz, 1H), 5.03 (s, 2H). | 409.0 |
| | 2,3-dichloro-N-(2- | | | |
| | (cyanomethyl)-1- | | | |
| | oxo-1,2- | | | |
| | dihydroisoquinolin- | | | |
| | 7- | | | |
| | yl)benzenesulfonamide | | | |
| Example 81 Method 1 | 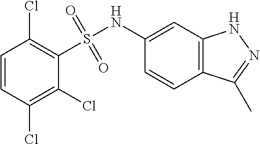 [SMILES: Cc1n[nH]c2cc(NS(=O)(=O)c3c(Cl)ccc(Cl)c3Cl)ccc12] 2,3,6-trichloro-N-(3- | Light- yellow solid, yield: 6.3% | ¹H NMR (400 MHz, DMSO-d₆) δ 12.50 (s, 1H), 11.03 (s, 1H), 7.85 (d, J = 8.8 Hz, 1H), 7.64 (d, J = 8.8 Hz, 1H), 7.57 (d, J = 8.6 Hz, 1H), 7.16 (d, J = 1.7 Hz, 1H), 6.87 (dd, J = 8.6, 1.8 Hz, 1H), 2.38 (s, 3H). | 389.9 |
| | methyl-1H-indazol- | | | |
| | 6- | | | |
| | yl)benzenesulfonamide | | | |
| Example 82 Method 1 | 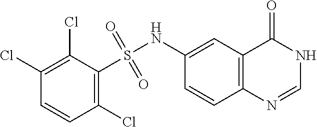 [SMILES: O=c1[nH]cnc2ccc(NS(=O)(=O)c3c(Cl)ccc(Cl)c3Cl)cc12] 2,3,6-trichloro-N-(4-oxo-3H-quinazolin-6- yl)benzenesulfonamide | Light- yellow solid, yield: 18.0% | ¹H NMR (400 MHz, DMSO-d₆) δ 12.22 (s, 1H), 11.38 (s, 1H), 7.99 (d, J = 3.1 Hz, 1H), 7.89 (d, J = 8.7 Hz, 1H), 7.82 (d, J = 2.5 Hz, 1H), 7.68 (d, J = 8.8 Hz, 1H), 7.61 (d, J = 8.8 Hz, 1H), 7.55 (dd, J = 8.8, 2.6 Hz, 1H). | 404.2 |
| Example 83 Method 1 | 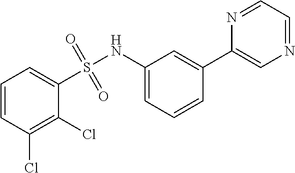 [SMILES: O=S(=O)(Nc1cccc(-c2cnccn2)c1)c1cccc(Cl)c1Cl] 2,3-dichloro-N-(3-pyrazin-2- | White solid, yield: 11.3% | ¹H NMR (400 MHz, DMSO-d₆) δ 11.01 (s, 1H), 9.12 (d, J = 1.6 Hz, 1H), 8.72 (dd, J = 2.5, 1.5 Hz, 1H), 8.62 (d, J = 2.5 Hz, 1H), 8.08 (dd, J = 8.0, 1.5 Hz, 1H), 7.93 − 7.87 (m, 2H), 7.81 − 7.75 (m, 1H), 7.54 (t, J = 8.0 Hz, 1H), 7.41 (t, J = 7.9 Hz, 1H), 7.29 − 7.19 (m, 1H). | 380.25 |
| | ylphenyl)benzenesulfonamide | | | |
| Example 84 Method 1 | 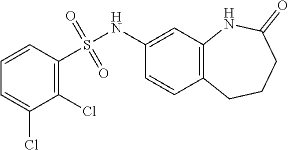 [SMILES: O=C1CCCc2ccc(NS(=O)(=O)c3cccc(Cl)c3Cl)cc2N1] | Yellow solid, yield: 77.2% | ¹H NMR (400 MHz, DMSO-d₆) δ 10.79 (s, 1H), 9.52 (s, 1H), 8.03 (dd, J = 8.0, 1.5 Hz, 1H), 7.92 (dd, J = 8.0, 1.5 Hz, 1H), 7.55 (t, J = 8.0 Hz, 1H), 7.08 (d, J = 8.0 Hz, 1H), 6.87 − 6.70 (m, 2H), 2.58 − 2.50 (m, 2H), 2.12 − 1.94 (m, 4H). | 385.0 |
| | 2,3-dichloro-N-(2- | | | |
| | oxo-2,3,4,5- | | | |
| | tetrahydro-1H- | | | |
| | benzo[b]azepin-8- | | | |
| | yl)benzenesulfonamide | | | |
| Example 85 Method 1 | 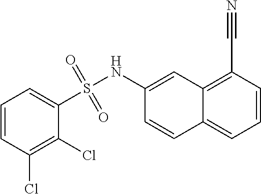 [SMILES: N#Cc1cccc2ccc(NS(=O)(=O)c3cccc(Cl)c3Cl)cc12] | White solid, yield: 38.2% | ¹H NMR (400 MHz, DMSO-d₆) δ 11.51 (s, 1H), 8.18 (d, J = 8.1 Hz, 2H), 8.09 (d, J = 7.1 Hz, 1H), 8.04 (d, J = 8.9 Hz, 1H), 7.94 (dd, J = 8.2, 1.5 Hz, 1H), 7.78 (d, J = 2.2 Hz, 1H), 7.60 (t, J = 8.1 Hz, 1H), 7.57 − 7.46 (m, 2H). | 377.1 |
| | 2,3-dichloro-N-(8- | | | |
| | cyanonaphthalen-2- | | | |
| | yl)benzenesulfonamide | | | |
| Example 86 Method 1 | 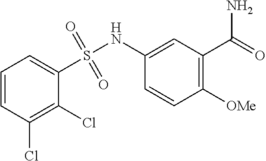 [SMILES: COc1ccc(NS(=O)(=O)c2cccc(Cl)c2Cl)cc1C(N)=O] | White solid, yield: 40% | ¹H NMR (400 MHz, DMSO-d₆) δ 10.58 (s, 1H), 7.92 (ddd, J = 8.4, 7.2, 1.6 Hz, 2H), 7.60 − 7.48 (m, 4H), 7.22 − 7.18 (m, 1H), 7.03 (d, J = 9.0 Hz, 1H), 3.80 (s, 3H). | 375.0 |
| | 5-((2,3- | | | |
| | dichlorophenyl)sulfonamido)-2- | | | |
| | methoxybenzamide | | | |
| Example 87 Method 1 | 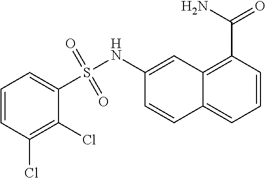 [SMILES: NC(=O)c1cccc2ccc(NS(=O)(=O)c3cccc(Cl)c3Cl)cc12] | White solid, yield: 15.2% | ¹H NMR (400 MHz, DMSO-d₆) δ 11.19 (s, 1H), 8.09 (dd, J = 8.0, 1.5 Hz, 1H), 8.03 (s, 1H), 7.93 − 7.72 (m, 4H), 7.52 (q, J = 8.1, 7.5 Hz, 3H), 7.41 − 7.27 (m, 2H). | 395.1 |
| | 7-((2,3- | | | |
| | dichlorophenyl) | | | |
| | sulfonamido)-1- | | | |
| | naphthamide | | | |
| Example 88 Method 1 | 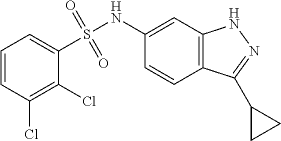 [SMILES: O=S(=O)(Nc1ccc2c(C3CC3)n[nH]c2c1)c1cccc(Cl)c1Cl] 2,3-dichloro-N-(3- cyclopropyl-1H- indazol-6- yl)benzenesulfonamide | White solid, yield: 25.6% | ¹H NMR (400 MHz, DMSO-d₆) δ 12.36 (s, 1H), 10.85 (s, 1H), 8.01 (dd, J = 8.0, 1.4 Hz, 1H), 7.89 (dd, J = 8.1, 1.4 Hz, 1H), 7.61 (d, J = 8.6 Hz, 1H), 7.52 (t, J = 8.0 Hz, 1H), 7.13 (d, J = 1.6 Hz, 1H), 6.87 (dd, J = 8.8, 2.0 Hz, 1H), 2.23 − 2.10 (m, 1H), 0.99 − 0.77 (m, 4H). | 382.0 |
| Example 89 Method 1 | 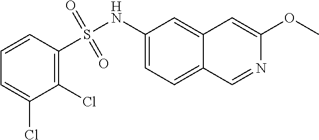 [SMILES: COc1cc2cc(NS(=O)(=O)c3cccc(Cl)c3Cl)ccc2cn1] 2,3-dichloro-N-(3- | White solid, yield: 3.43% | ¹H NMR (400 MHz, DMSO-d₆) δ 11.35 (s, 1H), 8.87 (s, 1H), 8.18 (dd, J = 8.0, 1.5 Hz, 1H), 7.95 − 7.87 (m, 2H), 7.57 (t, J = 8.0 Hz, 1H), 7.38 (d, J = 2.1 Hz, 1H), 7.21 (dd, J = 8.8, 2.1 Hz, 1H), 7.02 (s, 1H), 3.89 (s, 3H). | 383.2 |
| | methoxyisoquinolin-6- | | | |
| | yl)benzenesulfonamide | | | |
| Example 90 Method 1 Mixture of Stereoisomers | 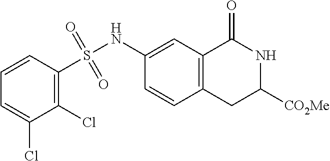 [SMILES: COC(=O)C1Cc2ccc(NS(=O)(=O)c3cccc(Cl)c3Cl)cc2C(=O)N1] methyl 7-((2,3- dichlorophenyl) | White solid, yield: 26% | ¹H NMR (400 MHz, DMSO-d₆) δ 10.94 (s, 1H), 8.18 (d, J = 4.2 Hz, 1H), 8.02 (dd, J = 8.0, 1.4 Hz, 1H), 7.93 (dd, J = 8.1, 1.5 Hz, 1H), 7.61 − 7.44 (m, 2H), 7.18 (s, 2H), 4.42 − 4.06 (m, 1H), 3.22 (dd, J = 16.3, 6.7 Hz, 1H), 3.02 (dd, J = 16.3, 3.4 Hz, 1H). | 428.83 |
| | sulfonamido)-1-oxo- | | | |
| | 1,2,3,4- | | | |
| | tetrahydroisoquinoline- | | | |
| | 3-carboxylate | | | |
| Example 91 Method 1 | 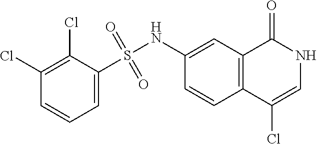 [SMILES: O=c1[nH]cc(Cl)c2ccc(NS(=O)(=O)c3cccc(Cl)c3Cl)cc12] 2,3-dichloro-N-(4- chloro-1-oxo-1,2- dihydroisoquinolin-7- | Light- yellow solid, yield: 8.8% | ¹H NMR (400 MHz, DMSO-d₆) δ 11.50 (d, J = 6.0 Hz, 1H), 11.27 (s, 1H), 8.07 (dd, J = 8.0, 1.6 Hz, 1H), 7.95 (d, J = 2.4 Hz, 1H), 7.92 (dd, J = 8.1, 1.5 Hz, 1H), 7.73 (d, J = 8.8 Hz, 1H), 7.62 (dd, J = 8.8, 2.5 Hz, 1H), 7.56 (t, J = 8.1 Hz, 1H), 7.35 (d, J = 6.1 Hz, 1H). | 403.3 |
| | yl)benzenesulfonamide | | | |
| Example 191 Method 1 | 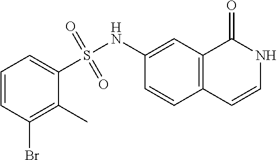 [SMILES: Cc1c(Br)cccc1S(=O)(=O)Nc1ccc2cc[nH]c(=O)c2c1] 3-bromo-2-methyl- N-(1-oxo-1,2- | Brown solid, yield: 73.8% | ¹H NMR (400 MHz, DMSO-d₆) δ 11.21 (d, J = 5.7 Hz, 1H), 10.91 (s, 1H), 7.93 (dd, J = 8.0, 1.2 Hz, 1H), 7.90 − 7.83 (m, 2H), 7.55 (d, J = 8.6 Hz, 1H), 7.43 (dd, J = 8.6, 2.4 Hz, 1H), 7.31 (t, J = 8.0 Hz, 1H), 7.06 (dd, J = 7.0, 5.7 Hz, 1H), 6.44 (d, J = 7.0 Hz, 1H), 2.69 (s, 3H). | 390.8 [M − H]⁻ |
| | dihydroisoquinolin-7- | | | |
| | yl)benzenesulfonamide | | | |
| Example 196 Method 1 Mixture of Stereoisomers | 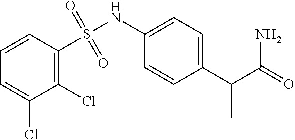 [SMILES: CC(C(N)=O)c1ccc(NS(=O)(=O)c2cccc(Cl)c2Cl)cc1] 2-(4-((2,3- dichlorophenyl)sulfonamido) phenyl)propanamide | White solid, yield: 10.1% | ¹H NMR (400 MHz, DMSO-d₆) δ 10.73 (s, 1H), 8.02 (dd, J = 8.0, 1.6 Hz, 1H), 7.91 (dd, J = 8.0, 1.6 Hz, 1H), 7.53 (t, J = 8.0 Hz, 1H), 7.31 (s, 1H), 7.16 (d, J = 8.4 Hz, 2H), 7.01 (d, J = 8.4 Hz, 2H), 6.76 (s, 1H), 3.44 (q, J = 7.0 Hz, 1H), 1.21 (d, J = 7.0 Hz, 3H). | 372.8 |
| Example 197 Method 1 | 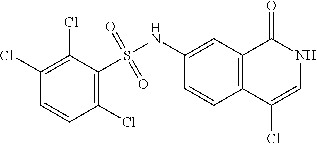 [SMILES: O=c1[nH]cc(Cl)c2ccc(NS(=O)(=O)c3c(Cl)ccc(Cl)c3Cl)cc12] 2,3,6-trichloro-N-(4- chloro-1-oxo-1,2-dihydroisoquinolin- 7-yl)benzenesulfonamide | White solid, yield: 5.7 % | ¹H NMR (400 MHz, DMSO-d6) δ 11.53 (d, J = 6.1 Hz, 1H), 11.48 (s, 1H), 7.97 (d, J = 2.4 Hz, 1H), 7.89 (d, J = 8.8 Hz, 1H), 7.75 (d, J = 8.8 Hz, 1H), 7.67 (d, J = 8.8 Hz, 1H), 7.61 (dd, J = 8.8, 2.5 Hz, 1H), 7.37 (d, J = 6.1 Hz, 1H). | 436.8 |
| Example 204 Method 1 | 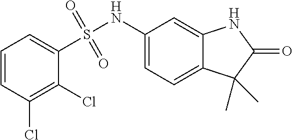 [SMILES: CC1(C)C(=O)Nc2cc(NS(=O)(=O)c3cccc(Cl)c3Cl)ccc21] | White solid, yield: 25.5% | ¹H NMR (400 MHz, DMSO-d₆) δ 10.78 (s, 1H), 10.24 (s, 1H), 8.01 (dd, J = 7.9, 1.5 Hz, 1H), 7.92 (dd, J = 8.1, 1.5 Hz, 1H), 7.55 (t, J = 8.0 Hz, 1H), 7.11 (d, J = 7.9 Hz, 1H), 6.70 − 6.64 (m, 2H), 1.15 (s, 6H). | 385.03 |
| | 2,3-dichloro-N-(3,3- | | | |
| | dimethyl-2-oxo- | | | |
| | indolin-6- | | | |
| | yl)benzenesulfonamide | | | |
| Example 206 Method 1 | 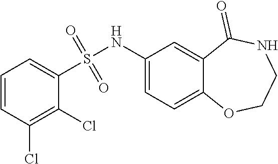 [SMILES: O=C1NCCOc2ccc(NS(=O)(=O)c3cccc(Cl)c3Cl)cc21] | White solid, yield: 15.9% | ¹H NMR (400 MHz, DMSO-d₆) δ 10.69 (s, 1H), 8.33 (t, J = 5.5 Hz, 1H), 8.00 − 7.86 (m, 2H), 7.56 − 7.49 (m, 2H), 7.18 (dd, J = 8.8, 2.9 Hz, 1H), 6.91 (d, J = 8.8 Hz, 1H), 4.20 (t, J = 4.8 Hz, 2H), 3.26 − 3.19 (m, 2H). | 386.94 |
| | 2,3-dichloro-N-(5- | | | |
| | oxo-3,4-dihydro-2H- | | | |
| | 1,4-benzoxazepin-7- | | | |
| | yl)benzenesulfonamide | | | |
| Example 208 Method 1 | 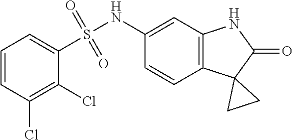 [SMILES: O=C1Nc2cc(NS(=O)(=O)c3cccc(Cl)c3Cl)ccc2C12CC2] 2,3-dichloro-N-(2′- oxospiro[cyclopropane-1,3′-indolin]-6′- yl)benzenesulfonamide | White solid, yield: 41.3% | ¹H NMR (400 MHz, DMSO-d₆) δ 10.67 (br s, 1H), 10.46 (br s, 1H), 7.96 (dd, J = 8.0, 1.5 Hz, 1H), 7.90 (dd, J = 8.1, 1.5 Hz, 1H), 7.52 (t, J = 8.0 Hz, 1H), 6.78 (d, J = 8.0 Hz, 1H), 6.72 (d, J = 1.9 Hz, 1H), 6.65 (dd, J = 8.0, 2.0 Hz, 1H), 1.48 − 1.40 (m, 2H), 1.38 − 1.30 (m, 2H). | 383.3 |
| Example 209 Method 1 | 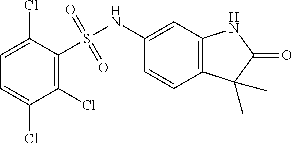 [SMILES: CC1(C)C(=O)Nc2cc(NS(=O)(=O)c3c(Cl)ccc(Cl)c3Cl)ccc21] | White solid, yield: 6.42% | ¹H NMR (400 MHz, DMSO-d₆) δ 10.93 (s, 1H), 10.31 (s, 1H), 7.90 (d, J = 8.8 Hz, 1H), 7.68 (d, J = 8.8 Hz, 1H), 7.14 (d, J = 8.6 Hz, 1H), 6.77 − 6.59 (m, 2H), 1.16 (s, 6H). | 419.3 |
| | 2,3,6-trichloro-N- | | | |
| | (3,3-dimethyl-2- | | | |
| | oxoindolin-6- | | | |
| | yl)benzenesulfonamide | | | |
| Example 211 Method 1 | 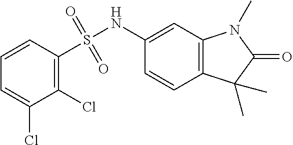 [SMILES: CN1C(=O)C(C)(C)c2ccc(NS(=O)(=O)c3cccc(Cl)c3Cl)cc21] | white solid, yield: 59.5% | ¹H NMR (400 MHz, DMSO-d₆) δ 10.84 (s, 1H), 8.09 (dd, J = 8.0, 1.5 Hz, 1H), 7.93 (dd, J = 8.2, 1.5 Hz, 1H), 7.56 (t, J = 8.0 Hz, 1H), 7.18 (d, J = 7.9 Hz, 1H), 6.76 − 6.68 (m, 2H), 3.03 (s, 3H), 1.17 (s, 6H). | 399.3 |
| | 2,3-dichloro-N- | | | |
| | (1,3,3-trimethyl-2- | | | |
| | oxoindolin-6- | | | |
| | yl)benzenesulfonamide | | | |
| Example 212 Method 1 | 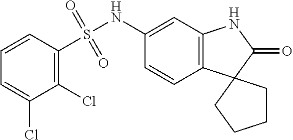 [SMILES: O=C1Nc2cc(NS(=O)(=O)c3cccc(Cl)c3Cl)ccc2C12CCCC2] 2,3-dichloro-N-(2′-oxospiro [cyclopentane-1,3′-indolin]-6′- | brown solid, yield: 52.9% | ¹H NMR (400 MHz, DMSO-d₆) δ 10.78 (s, 1H), 10.20 (s, 1H), 8.01 (dd, J = 7.9, 1.5 Hz, 1H), 7.93 (dd, J = 8.1, 1.5 Hz, 1H), 7.56 (t, J = 8.0 Hz, 1H), 7.07 (d, J = 7.9 Hz, 1H), 6.70 − 6.62 (m, 2H), 1.93 − 1.78 (m, 6H), 1.70 − 1.57 (m, 2H). | 411.3 |
| | yl)benzenesulfonamide | | | |
| Example 217 Method 1 | 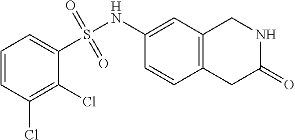 [SMILES: O=C1Cc2ccc(NS(=O)(=O)c3cccc(Cl)c3Cl)cc2CN1] | White solid, yield: 6.60% | ¹H NMR (400 MHz, DMSO-d₆) δ 10.75 (s, 1H), 8.07 − 7.99 (m, 1H), 7.97 − 7.86 (m, 2H), 7.60 − 7.48 (m, 1H), 7.15 − 6.90 (m, 3H), 4.21 (s, 2H), 3.30 (s, 2H). | 370.93 |
| | 2,3-dichloro-N-(3- | | | |
| | oxo-1,2,3,4- | | | |
| | tetrahydroisoquinolin-7- | | | |
| | yl)benzenesulfonamide | | | |
| Example 219 Method 1 | 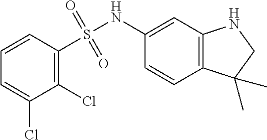 [SMILES: CC1(C)CNc2cc(NS(=O)(=O)c3cccc(Cl)c3Cl)ccc21] 2,3-dichloro-N-(3,3- | yellow solid. | ¹H NMR (400 MHz, DMSO-d₆) δ 10.37 (s, 1H), 7.98 (dd, J = 7.9, 1.6 Hz, 1H), 7.91 (dd, J = 8.0, 1.5 Hz, 1H), 7.54 (t, J = 8.0 Hz, 1H), 6.77 (d, J = 7.7 Hz, 1H), 6.31 − 6.22 (m, 2H), 5.57 (s, 1H), 3.09 (d, J = 1.5 Hz, 2H), 1.12 (s, 6H). | 371.3 |
| | dimethylindolin-6- | | | |
| | yl)benzenesulfonamide | | | |
| Example 220 Method 1 | 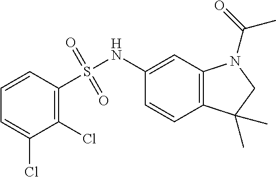 [SMILES: CC(=O)N1CC(C)(C)c2ccc(NS(=O)(=O)c3cccc(Cl)c3Cl)cc21] | red solid, yield: 59.4% | ¹H NMR (400 MHz, DMSO-d₆) δ 10.66 (s, 1H), 8.00 (dd, J = 8.0, 1.5 Hz, 1H), 7.95 − 7.85 (m, 2H), 7.54 (t, J = 8.0 Hz, 1H), 7.07 (d, J = 8.1 Hz, 1H), 6.75 (dd, J = 8.1, 2.1 Hz, 1H), 3.78 (s, 2H), 2.10 (s, 3H), 1.20 (s, 6H). | 413.04 |
| | N-(1-acetyl-3,3- | | | |
| | dimethyl-indolin-6- | | | |
| | yl)-2,3-dichloro- | | | |
| | benzenesulfonamide | | | |
| Example 224 Method 1 | 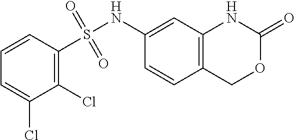 [SMILES: O=C1Nc2cc(NS(=O)(=O)c3cccc(Cl)c3Cl)ccc2CO1] | white solid, yield: 12.23% | ¹H NMR (400 MHz, DMSO) δ 10.88 (s, 1H), 10.11 (s, 1H), 8.01 (dd, J = 8.0, 1.1 Hz, 1H), 7.93 (d, J = 7.8 Hz, 1H), 7.56 (t, J = 8.0 Hz, 1H), 7.03 (d, J = 8.0 Hz, 1H), 6.71 (m, 2H), 5.14 (s, 2H). | 373.0 |
| | 2,3-dichloro-N-(2- | | | |
| | oxo-1,4-dihydro-2H- | | | |
| | benzo[d][1,3]oxazin- | | | |
| | 7- | | | |
| | yl)benzenesulfonamide | | | |
| Example 225 Method 1 | 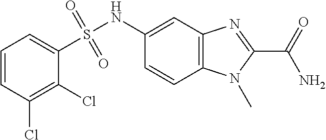 [SMILES: Cn1c(C(N)=O)nc2cc(NS(=O)(=O)c3cccc(Cl)c3Cl)ccc21] 5-((2,3- | White solid, yield: 19.0% | ¹H NMR (400 MHz, DMSO-d₆) δ 10.60 (s, 1H), 8.17 (s, 1H), 7.95 (dd, J = 8.0, 1.6 Hz, 1H), 7.82 (d, J = 7.6 Hz, 1H), 7.74 (s, 1H), 7.49 (d, J = 8.8 Hz, 1H), 7.44 (t, J = 8.0 Hz, 1H), 7.32 (s, 1H), 7.10 (d, J = 8.3 Hz, 1H), 4.01 (s, 3H). | 398.9 |
| | dichlorophenyl)sulfonamido)- | | | |
| | 1-methyl- | | | |
| | 1H- | | | |
| | benzo[d]imidazole-2- | | | |
| | carboxamide | | | |
| Example 227 Method 1 | 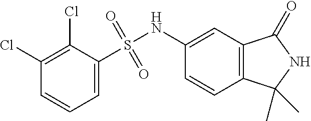 [SMILES: CC1(C)NC(=O)c2cc(NS(=O)(=O)c3cccc(Cl)c3Cl)ccc21] 2,3-dichloro-N-(1,1- | White solid, yield: 38.1% | ¹H NMR (400 MHz, DMSO-d₆) δ 11.03 (s, 1H), 8.67 (s, 1H), 8.06 (dd, J = 8.0, 1.6 Hz, 1H), 7.94 (dd, J = 8.1, 1.5 Hz, 1H), 7.57 (t, J = 8.0 Hz, 1H), 7.50 (d, J = 8.1 Hz, 1H), 7.36 − 7.23 (m, 2H), 1.35 (s, 6H). | 385.1 |
| | dimethyl-3- | | | |
| | oxoisoindolin-5- | | | |
| | yl)benzenesulfonamide | | | |
| Example 251 Method 1 | 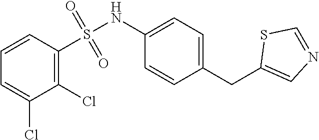 [SMILES: O=S(=O)(Nc1ccc(Cc2cncs2)cc1)c1cccc(Cl)c1Cl] | white solid, yield: 1.3% | ¹H NMR (400 MHz, Methanol-d₄) δ 8.74 (s, 1H), 7.88 (dd, J = 8.0, 1.5 Hz, 1H), 7.63 (dd, J = 8.0, 1.5 Hz, 1H), 7.49 (s, 1H), 7.28 (t, J = 8.0 Hz, 1H), 7.07 − 6.92 (m, 4H), 4.01 (s, 2H). | 399.0 |
| | 2,3-dichloro-N-(4- | | | |
| | (thiazol-5- | | | |
| | ylmethyl)phenyl) | | | |
| | benzenesulfonamide | | | |
| Example 264 Method 1 | 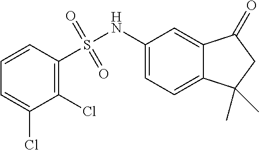 [SMILES: CC1(C)CC(=O)c2cc(NS(=O)(=O)c3cccc(Cl)c3Cl)ccc21] 2,3-dichloro-N-(1,1- | white solid, yield: 26.9% | ¹H NMR (400 MHz, DMSO-d₆) δ 10.67 (s, 1H), 8.01 (d, J = 1.4 Hz, 1H), 7.92 (s, 1H), 7.55 (s, 1H), 7.05 (d, J = 8.3 Hz, 2H), 6.96 (dd, J = 8.1, 2.0 Hz, 1H), 2.15 (dd, J = 12.5, 7.0 Hz, 1H), 1.62 (dd, J = 12.5, 7.2 Hz, 1H), 1.23 (s, 3H), 1.05 (s, 3H). | 384 |
| | dimethyl-3-oxo-2,3- | | | |
| | dihydro-1H-inden-5- | | | |
| | yl)benzenesulfonamide | | | |
| Example 265 Method 1 | 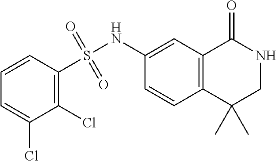 [SMILES: CC1(C)CNC(=O)c2cc(NS(=O)(=O)c3cccc(Cl)c3Cl)ccc21] | white solid, yield: 36.2% | ¹H NMR (400 MHz, Methanol-d₄): δ 8.06 (dd, J = 8.0 Hz, 1.6 Hz, 1H), 7.75 (dd, J = 8.4 Hz, 1.6 Hz, 1H), 7.71 (d, J = 2.4 Hz, 1H), 7.41 (t, J = 8.0 Hz, 1H), 7.35-7.27 (m, 2H), 3.20 (s, 2H), 1.26 (s, 6H). | 398.9 |
| | 2,3-dichloro-N-(4,4- | | | |
| | dimethyl-1-oxo- | | | |
| | 1,2,3,4- | | | |
| | tetrahydroisoquinolin-7- | | | |
| | yl)benzenesulfonamide | | | |
| Example 268 Method 1 | 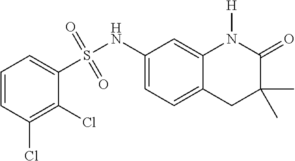 [SMILES: CC1(C)Cc2ccc(NS(=O)(=O)c3cccc(Cl)c3Cl)cc2NC1=O] 2,3-dichloro-N-(3,3- | White solid, yield: 49.2% | ¹H NMR (400 MHz, DMSO-d₆) δ 10.67 (s, 1H), 9.99 (s, 1H), 7.99 (dd, J = 8.0, 1.5 Hz, 1H), 7.92 (dd, J = 8.1, 1.5 Hz, 1H), 7.54 (t, J = 8.0 Hz, 1H), 6.98 (d, J = 8.1 Hz, 1H), 6.69 (d, J = 2.2 Hz, 1H), 6.62 (dd, J = 8.0, 2.2 Hz, 1H), 2.60 (s, 2H), 0.97 (s, 6H). | 399.04 |
| | dimethyl-2-oxo-1,4- | | | |
| | dihydroquinolin-7- | | | |
| | yl)benzenesulfonamide | | | |
| Example 269 Method 1 Mixture of Stereoisomers | 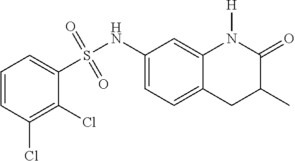 [SMILES: CC1Cc2ccc(NS(=O)(=O)c3cccc(Cl)c3Cl)cc2NC1=O] 2,3-dichloro-N-(3- methyl-2-oxo-3,4- dihydro-1H-quinolin-7- yl)benzenesulfonamide | White solid, yield: 12.7% | ¹H NMR (400 MHz, DMSO-d₆) δ 10.69 (s, 1H), 10.02 (s, 1H), 8.00 (dd, J = 7.9, 1.5 Hz, 1H), 7.92 (dd, J = 8.1, 1.5 Hz, 1H), 7.54 (t, J = 8.0 Hz, 1H), 6.99 (d, J = 8.1 Hz, 1H), 6.69 (d, J = 2.2 Hz, 1H), 6.61 (dd, J = 8.1, 2.2 Hz, 1H), 2.79 (dd, J = 15.0, 5.4 Hz, 1H), 2.48 − 2.34 (m, 2H), 1.05 (d, J = 6.6 Hz, 3H). | 385.03 |
| Example 270 Method 1 single unknown stereoisomer | 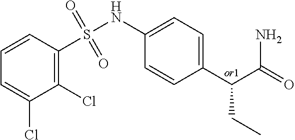 [SMILES: CC[C@@H](C(N)=O)c1ccc(NS(=O)(=O)c2cccc(Cl)c2Cl)cc1] rel-(R)-2-(4-((2,3-dichlorophenyl) sulfonamido)phenyl)butanamide | white solid, yield: 5.7% | ¹H NMR (400 MHz, DMSO-d₆) δ 10.70 (s, 1H), 8.01 (dd, J = 8.0, 1.6 Hz, 1H), 7.90 (dd, J = 8.2, 1.2 Hz, 1H), 7.53 (t, J = 8.0 Hz, 1H), 7.37 (s, 1H), 7.15 (d, J = 8.6 Hz, 2H), 7.00 (d, J = 8.6 Hz, 2H), 6.76 (s, 1H), 3.18 (m, 1H), 1.83 (m, 1H), 1.47 (m, 1H), 0.74 (t, J = 7.4 Hz, 3H). | 386.9 Note: SFC Thar prep 80 CHIRALPAK AD-H 250 mm × 20 mm, 5 μm Modifier: 40% EtOH (NH₄OH 0.2%) |
| | | | | Rt = 4.2 min |
| Example 270A Method 1 single unknown stereoisomer | 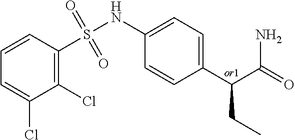 [SMILES: CC[C@H](C(N)=O)c1ccc(NS(=O)(=O)c2cccc(Cl)c2Cl)cc1] rel-(S)-2-(4-((2,3-dichlorophenyl) sulfonamido)phenyl)butanamide | white solid, yield: 4.2% | ¹H NMR (400 MHz, DMSO-d₆) δ 10.70 (s, 1H), 8.01 (dd, J = 8.0, 1.6 Hz, 1H), 7.90 (dd, J = 8.2, 1.2 Hz, 1H), 7.53 (t, J = 8.0 Hz, 1H), 7.37 (s, 1H), 7.15 (d, J = 8.6 Hz, 2H), 7.00 (d, J = 8.6 Hz, 2H), 6.76 (s, 1H), 3.18 (m, 1H), 1.83 (m, 1H), 1.47 (m, 1H), 0.74 (t, J = 7.4 Hz, 3H). | 386.9 Note: SFC Thar prep 80 CHIRALPAK AD-H 250 mm × 20 mm, 5 μm Modifier : 40% EtOH (NH₄OH 0.2%) Rt = 3.74 min |
| | | | | 386 |
| Example 274 Method 1 Mixture of Stereoisomers | 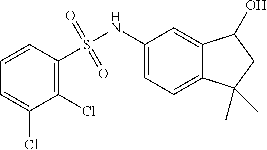 [SMILES: CC1(C)CC(O)c2cc(NS(=O)(=O)c3cccc(Cl)c3Cl)ccc21] | white solid, yield: 25.8% | ¹H NMR (400 MHz, DMSO-d₆) δ 10.67 (s, 1H), 8.02 (dd, J = 8.0, 1.5 Hz, 1H), 7.92 (dd, J = 8.1, 1.5 Hz, 1H), 7.55 (t, J = 8.0 Hz, 1H), 7.07 − 7.01 (m, 2H), 6.96 (dd, J = 8.1, 2.0 Hz, 1H), 5.21 (d, J = 6.2 | |
| | | | Hz, 1H), 4.95 (q, J = | |
| | 2,3-dichloro-N-(3- | | 6.8 Hz, 1H), 2.15 (dd, J | |
| | hydroxy-1,1- | | = 12.5, 7.0 Hz, 1H), | |
| | dimethyl-2,3- | | 1.62 (dd, J = 12.5, 7.2 | |
| | dihydro-1H-inden-5- | | Hz, 1H), 1.23 (s, 3H), | |
| | yl)benzenesulfonamide | | 1.05 (s, 3H). | |
| Example 283 Method 1 Mixture of Stereoisomers | 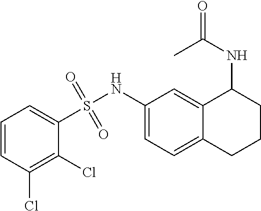 [SMILES: CC(=O)NC1CCCc2ccc(NS(=O)(=O)c3cccc(Cl)c3Cl)cc21] | White solid, yield: 15.3% | ¹H NMR (400 MHz, DMSO-d₆) δ 8.06 (d, J = 8.9 Hz, 1H), 6.81 (d, J = 8.3 Hz, 1H), 6.45 (dd, J = 8.4, 2.5 Hz, 1H), 6.40 − 6.28 (m, 2H), 4.83 (s, 1H), 4.57 (p, J = 6.6 Hz, 1H), 2.56-2.53 (m, 2H), 1.83 − 1.78 (m, 2H), 1.77 (s, 3H), 1.49 (d, J = 6.8 Hz, 3H). | 413.1 |
| | N-(7-((2,3- | | | |
| | dichlorophenyl)sulfonamido)-1,2,3,4- | | | |
| | tetrahydronaphthalen- | | | |
| | 1-yl)acetamide | | | |
| Example 298 Method 1 Mixture of Stereoisomers | 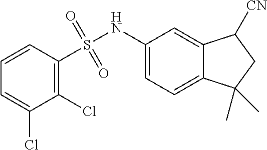 [SMILES: CC1(C)CC(C#N)c2cc(NS(=O)(=O)c3cccc(Cl)c3Cl)ccc21] 2,3-dichloro-N-(3-cyano-1,1-dimethyl- 2,3-dihydro-1H-inden-5-yl)benzenesulfonamide | white solid, yield: 51.9% | ¹H NMR (400 MHz, DMSO-d₆) δ 10.86 (s, 1H), 8.05 (dd, J = 7.9, 1.5 Hz, 1H), 7.94 (dd, J = 8.1, 1.5 Hz, 1H), 7.56 (t, J = 8.0 Hz, 1H), 7.19 − 7.02 (m, 3H), 4.47 (t, J = 8.0 Hz, 1H), 2.30 (dd, J = 12.5, 8.1 Hz, 1H), 2.04 (dd, J = 12.6, 8.0 Hz, 1H), 1.26 (s, 3H), 1.09 (s, 3H). | 395 |
| Example 304A Method 1 Mixture of Stereoisomers | 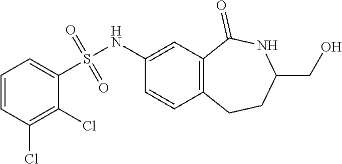 [SMILES: O=C1NC(CO)CCc2ccc(NS(=O)(=O)c3cccc(Cl)c3Cl)cc21] | white solid, yield: 14.7% | ¹H NMR (400 MHz, DMSO-d₆) δ 10.87 (s, 1H), 8.02 (dd, J = 7.9, 1.5 Hz, 1H), 7.93 (dd, J = 8.1, 1.5 Hz, 1H), 7.62 (d, J = 5.2 Hz, 1H), 7.55 (t, J = 8.0 Hz, 1H), 7.24 (t, J = 1.5 Hz, 1H), 7.13 (d, J = 1.5 Hz, 2H), 4.64 (t, J = 5.8 Hz, | 415.3 |
| | | | 1H), 3.38 (t, J = 5.8 Hz, | |
| | 2,3-dichloro-N-(3- | | 2H), 2.92-2.82 (m, 1H), | |
| | (hydroxymethyl)-1- | | 2.64-2.56 (m, 2H), | |
| | oxo-2,3,4,5- | | 1.77-1.65 (m, 2H). | |
| | tetrahydro-1H- | | | |
| | benzo[c]azepin-8- | | | |
| | yl)benzenesulfonamide | | | |
| Example 304 Method 1 single unknown stereoisomer | 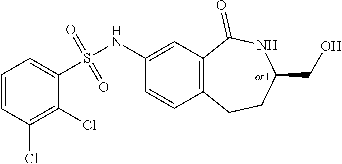 [SMILES: O=C1N[C@@H](CO)CCc2ccc(NS(=O)(=O)c3cccc(Cl)c3Cl)cc21] | white solid, yield 14.7% | ¹H NMR (400 MHz, DMSO-d₆) δ 10.87 (s, 1H), 8.02 (dd, J = 7.9, 1.5 Hz, 1H), 7.93 (dd, J = 8.1, 1.5 Hz, 1H), 7.62 (d, J = 5.2 Hz, 1H), 7.55 (t, J = 8.0 Hz, 1H), 7.24 (t, J = 1.5 Hz, 1H), 7.13 (d, J = 1.5 Hz, 2H), 4.64 (t, J = 5.8 Hz, | 415.3 |
| | | | 1H), 3.38 (t, J = 5.8 Hz, | |
| | Rel-(S)-2,3-dichloro- | | 2H), 2.92-2.82 (m, 1H), | |
| | N-(3- | | 2.64-2.56 (m, 2H), | |
| | (hydroxymethyl)-1- | | 1.77-1.65 (m, 2H). | |
| | oxo-2,3,4,5- | | | |
| | tetrahydro-1H- | | | |
| | benzo[c]azepin-8- | | | |
| | yl)benzenesulfonamide | | | |
| Example 304B Method 1 single unknown stereoisomer | 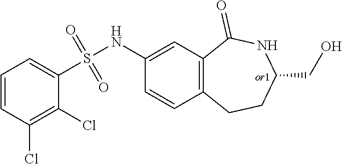 [SMILES: O=C1N[C@H](CO)CCc2ccc(NS(=O)(=O)c3cccc(Cl)c3Cl)cc21] rel-(R)-2,3-dichloro-N-(3- (hydroxymethyl)-1-oxo-2,3,4,5- tetrahydro-1H-benzo[c]azepin-8- yl)benzenesulfonamide | White solid, yield: 14.7% | ¹H NMR (400 MHz, DMSO-d₆) δ 10.87 (s, 1H), 8.02 (dd, J = 7.9, 1.5 Hz, 1H), 7.93 (dd, J = 8.1, 1.5 Hz, 1H), 7.62 (d, J = 5.2 Hz, 1H), 7.55 (t, J = 8.0 Hz, 1H), 7.24 (t, J = 1.5 Hz, 1H), 7.13 (d, J = 1.5 Hz, 2H), 4.64 (t, J = 5.8 Hz, 1H), 3.38 (t, J = 5.8 Hz, 2H), 2.92-2.82 (m, 1H), 2.64-2.56 (m, 2H), 1.77-1.65 (m, 2H). | 415.29 |

### Preparation of 6-(5-(pyridazin-3-ylamino)pyridin-3-yl)benzo[d]oxazol-2(3H)-one (1A) Method 2

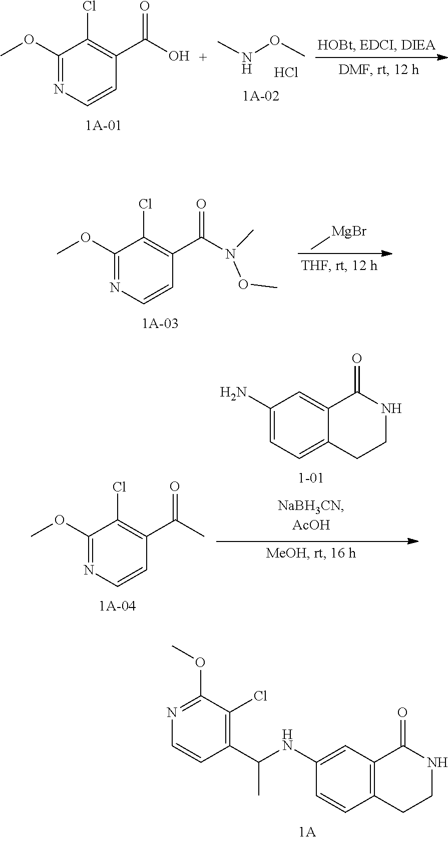

Step 1: A mixture of 1A-01 (500 mg, 5.33 mmol), 1A-02 (270 mg, 5.33 mmol), HOBt (720 mg, 5.33 mmol), EDCI (1.023 g, 5.33 mmol) and DIEA (2.07 g, 16 mmol) in DMF (10 mL) was stirred under N₂ at 25° C. for 12 h. The reaction mixture was diluted with water. The aqueous phase was extracted with EA. The combined organic extracts were washed with brine and dried over Na₂SO₄. The solvent was removed under vacuum to give 600 mg of a crude product 1A-03 that was used in the next step directly.

Step 2: 1A-03 (600 mg, 5.33 mmol) was dissolved in 10 mL of dry THF, methylmagnesium bromide (2 M in THF, 6 mL, 12 mmol) was added to the above solution at 0° C., and the mixture was stirred at rt for 12 h. The mixture was quenched with a saturated ammonium chloride solution and extracted with EA, washed with brine, dried over (Na₂SO₄), and concentrated in vacuo. Purification by silica gel chromatography gave the compound 1A-04 (300 mg, 49%).

Step 3: 1A-04 (90 mg, 0.5 mmol) was dissolved in 5 mL of dry MeOH, 1A-05 (240 mg, 1.5 mmol), NaBH₃CN (0.28 g, 4.5 mmol) and one drop of AcOH were added to the above solution, and the mixture was stirred for 12 h at r.t. The mixture was quenched with water and extracted with EA, washed with brine, dried over (Na₂SO₄), and concentrated in vacuo. Purification by silica gel chromatography gave the titled compound 1A (14 mg, 8.1%). ¹H NMR (400 MHz, DMSO-d₆) δ 8.12 (d, J=2.4 Hz, 1H), 7.87 (d, J=2.4 Hz, 1H), 7.71 (s, 1H), 7.05 (s, 1H), 6.95 (d, J=8.0 Hz, 1H), 6.66 (d, J=8.0 Hz, 1H), 6.22 (s, 1H), 4.56 (d, J=6.8 Hz, I H), 3.89 (s, 3H), 3.25 (td, J=6.4, 2.8 Hz, 2H), 2.67 (t, J=6.4 Hz, 2H), 1.41 (d, J=6.8 Hz, 3H).

### Preparation of (S)-7-((1-(3-chlorophenyl)ethyl)amino)isoquinolin-1-ol (92)

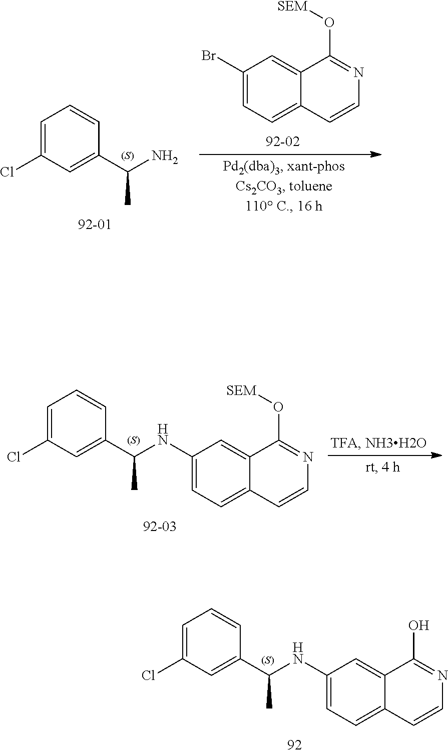

Step 1: A solution of 92-01 (200 mg, 1.29 mmol) in toluene (5 mL) under argon was added Pd2(dba)3 (590 mg, 0.64 mmol), Xant-phos (373 mg, 0.64 mmol), 92-02 (455 mg, 1.28 mmol) and Cs₂CO₃ (1300 mg, 3.99 mmol), and the mixture was degassed with argon. The mixture was heated at 110° C. for 16h. Upon completion, the mixture was diluted with EtOAc, filtered through Celite and concentrated in vacuo. The crude product was purified by flash column chromatography to give the product 92-03. MS (m/z): 429.1 (M+H+).

Step 2 Compound 92-03 (200 mg, 0.46 mmol) in DCM (4 mL) was stirred at r.t. followed by addition of TFA (1 mL). The reaction mixture was stirred at 25° C. for 2 h and LCMS showed the reaction was completed. The solvent was removed in vacuo and NH₃·H₂O (2 mL) was added. The mixture was stirred at room temperature for 2 h. The residue was concentrated under vacuum and purified by prep-HPLC to afford 92 (45 mg, 32.6 yield) as a light-yellow solid. MS (m/z): 299.0 [M+H]⁺. ¹H NMR (400 MHz, Methanol-d₄) δ 7.44 (t, J=1.9 Hz, 1H), 7.41 (d, J=8.6 Hz, 1H), 7.37 (dt, J=7.6, 1.6 Hz, 1H), 7.29 (t, J=7.8 Hz, 1H), 7.24-7.17 (m, 2H), 7.13 (dd, J=8.6, 2.5 Hz, 1H), 6.89 (d, J=7.0 Hz, 1H), 6.55 (d, J=7.0 Hz, 1H), 4.62 (q, J=6.8 Hz, 1H), 1.55 (d, J=6.9 Hz, 3H).

Examples (Compounds) 93-157, 192-195, 198-201A, 203, 205, 210, 213, 221-223, 237-237A, 239A-239B, 244 and 254 were synthesized using a method like that used in Example 1A.

| Example | | Appear- | | |
|---|---|---|---|---|
| (Compound) | | ance | | |
| No.) | | and | | MS (m/z) |
| Method | Structure and Name | Yield | ¹H NMR Data | [M + H]⁺ |
| Example 93 Method 2 Mixture of Stereoisomers | 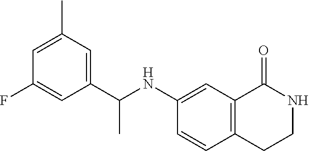 [SMILES: Cc1cc(F)cc(C(C)Nc2ccc3c(c2)C(=O)NCC3)c1] | White solid, yield: 7.96% | ¹H NMR (400 MHz, Chloroform-d) δ 7.33 (s, 1H), 6.94 (d, J = 8.4 Hz, 2H), 6.87 (d, J = 9.6 Hz, 1H), 6.72 (d, J = 9.6 Hz, 1H), 6.60 (d, J = 8.0 Hz, 1H), 5.97 (s, 1H), 4.5 (q, J = 6.4 Hz, 1H), 3.48 (td, J = 6.8, 2.4 Hz, 2H), 2.84 (t, J = | 299.1 |
| | 7-((1-(3-fluoro-5- | | 6.4 Hz, 2H), 2.32 (s, | |
| | methylphenyl)ethyl) | | 3H), 1.52 (d, J = 6.8 | |
| | amino)-3,4- | | Hz, 3H). | |
| | dihydroisoquinolin- | | | |
| | 1(2H)-one | | | |
| Example 94 Method 2 Mixture of Stereoisomers | 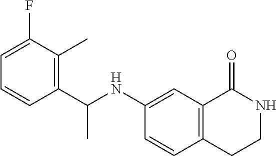 [SMILES: Cc1c(F)cccc1C(C)Nc1ccc2c(c1)C(=O)NCC2] | White solid, yield: 21% | ¹H NMR (400 MHz, DMSO-d₆) δ 7.67 (br, 1H), 7.19 (dd, J = 1.6, 7.6 Hz, 1H), 7.15-7.09 (m, 1H), 7.00-6.93 (m, 2H), 6.91 (d, J = 8.4 Hz, 1H), 6.52 (dd, J = 8.4, 2.8 Hz, 1H), 6.31 (d, J = 6.8 Hz, 1H), 4.65 (t, J = 6.8 Hz, 1H), | 300.1 |
| | 2-((1-(3-fluoro-2- | | 3.26 (td, J = 6.4, 2.8 | |
| | methylphenyl)ethyl) | | Hz, 2H), 2.64 (t, J = 6.4 | |
| | amino)-6,7-dihydro- | | Hz, 2H), 2.31 (d, J = | |
| | 1,7-naphthyridin- | | 2.0 Hz, 3H), 1.37 (d, | |
| | 8(5H)-one | | J = 6.8 Hz, 3H). | |
| Example 95 Method 2 single unknown stereoisomer | 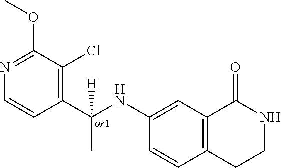 [SMILES: COc1nccc([C@H](C)Nc2ccc3c(c2)C(=O)NCC3)c1Cl] | White solid, yield: 36% | ¹H NMR (400 MHz, DMSO-d₆) δ 8.13 (d, J = 2.4 Hz, 1H), 7.87 (d, J = 2.4 Hz, 1H), 7.71 (s, 1H), 7.05 (d, J = 2.4 Hz, 1H), 6.96 (d, J = 8.0 Hz, 1H), 6.67 (d, J = 8.0 Hz, 1H), 6.22 (br, 1H), 4.57 (q, J = 6.4 Hz, 1H), 3.89 (s, 3H), 3.25 (td, J = 6.4, 2.8 | 332.1 Note: SFC Thar prep 80 CHIRALPAK AS-H 250 mm * 20 mm, 5 μm Modifier: 40% ETOH (NH₄OH |
| | rel-(S)-7-((1-(3- | | Hz, 2H), 2.73-2.67 (t, | 0.2%). Rt = |
| | chloro-2- | | J = 6.4 Hz, 2H), 1.42 | 4.17 min |
| | methoxypyridin-4- | | (d, J = 6.8 Hz, 3H). | |
| | yl)ethyl)amino)-3,4- | | | |
| | dihydroisoquinolin- | | | |
| | 1(2H)-one | | | |
| Example 96 Method 2 Mixture of Stereoisomers | 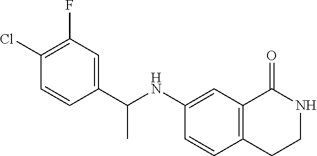 [SMILES: CC(Nc1ccc2c(c1)C(=O)NCC2)c1ccc(Cl)c(F)c1] | White solid, yield: 6.7% | ¹H NMR (400 MHz, DMSO-d₆) δ 7.68 (t, J = 2.8 Hz, 1H), 7.50 (t, J = 8.0 Hz, 1H), 7.39 (dd, J = 10.5, 2.0 Hz, 1H), 7.24 (dd, J = 8.2, 2.0 Hz, 1H), 7.02 (d, J = 2.6 Hz, 1H), 6.92 (d, J = 8.2 Hz, 1H), 6.61 (dd, J = 8.2, 2.6 Hz, 1H), | 319.3 |
| | 7-((1-(4-chloro-3- | | 6.27 (d, J = 7.3 Hz, | |
| | fluorophenyl)ethyl) | | 1H), 4.53 (t, J = 6.9 Hz, | |
| | amino)-3,4- | | 1H), 3.25 (td, J = 6.6, | |
| | dihydroisoquinolin- | | 2.7 Hz, 2H), 2.66 (t, J = | |
| | 1(2H)-one | | 6.5 Hz, 2H), 1.40 (s, | |
| | | | 3H). | |
| Example 97 Method 2 Mixture of Stereoisomers | 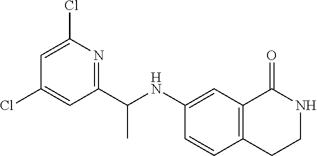 [SMILES: CC(Nc1ccc2c(c1)C(=O)NCC2)c1cc(Cl)cc(Cl)n1] | White solid, yield: 9.7% | ¹H NMR (400 MHz, DMSO-d₆) δ 7.73 (s, 1H), 7.63 (d, J = 1.6 Hz, 1H), 7.49 (d, J = 1.6 Hz, 1H), 7.03 (d, J = 2.6 Hz, 1H), 6.97 (d, J = 8.2 Hz, 1H), 6.63 (dd, J = 8.2, 2.6 Hz, 1H), 4.52 (q, J = 6.8 Hz, 1H), 3.26 (td, J = | 336.2 |
| | 7-((1-(4,6- | | 6.6, 2.8 Hz, 2H), 2.68 | |
| | dichloropyridin-2- | | (t, J = 6.5 Hz, 2H), 1.44 | |
| | yl)ethyl)amino)-3,4- | | (d, J = 6.8 Hz, 3H). | |
| | dihydroisoquinolin- | | | |
| | 1(2H)-one | | | |
| Example 98 Method 2 Mixture of Stereoisomers | 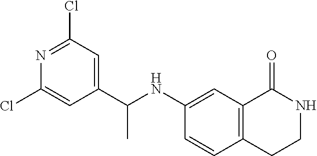 [SMILES: CC(Nc1ccc2c(c1)C(=O)NCC2)c1cc(Cl)nc(Cl)c1] | White solid, yield: 3.06% | ¹H NMR (400 MHz, DMSO-d₆) δ 7.74 (d, J = 3.0 Hz, 1H), 7.58 (s, 2H), 7.02 (d, J = 2.6 Hz, 1H), 6.97 (d, J = 8.2 Hz, 1H), 6.63 (dd, J = 8.2, 2.6 Hz, 1H), 6.35 (d, J = 7.8 Hz, 1H), 4.63 (p, J = 6.8 Hz, 1H), 3.26 (dd, J = 6.6, | 336.2 |
| | 7-((1-(2,6- | | 2.6 Hz, 2H), 2.68 (d, | |
| | dichloropyridin-4- | | J = 6.6 Hz, 2H), 1.42 (d, | |
| | yl)ethyl)amino)-3,4- | | J = 6.8 Hz, 3H). | |
| | dihydroisoquinolin- | | | |
| | 1(2H)-one | | | |
| Example 99 Method 2 Mixture of Stereoisomers | 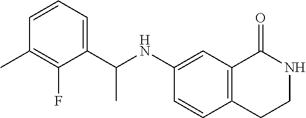 [SMILES: Cc1cccc(C(C)Nc2ccc3c(c2)C(=O)NCC3)c1F] | White solid, yield: 3.6% | ¹H NMR (400 MHz, DMSO-d₆) δ 7.72 (s, 1H), 7.20-7.13 (m, 1H), 7.10 (d, J = 6.8 Hz, 1H), 7.02-6.95 (m, 2H), 6.92 (d, J = 8.2 Hz, 1H), 4.73 (p, J = 6.8 Hz, 1H), 3.24 (dd, | 299.2 |
| | 7-((1-(2-fluoro-3- | | J = 6.6, 2.8 Hz, 2H), | |
| | methylphenyl)ethyl) | | 2.65 (d, J = 6.6 Hz, | |
| | amino)-3,4- | | 2H), 2.24 (d, J = 2.0 | |
| | dihydroisoquinolin- | | Hz, 3H), 1.42 (d, J = | |
| | 1(2H)-one | | 6.8 Hz, 3H). | |
| Example 100 Method 2 Mixture of Stereoisomers | 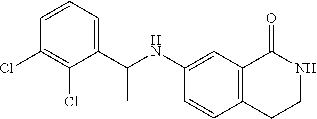 [SMILES: CC(Nc1ccc2c(c1)C(=O)NCC2)c1cccc(Cl)c1Cl] | White solid, yield: 5.1% | ¹H NMR (400 MHz, DMSO-d₆) δ 7.72 (s, 1H), 7.49 (d, J = 1.6 Hz, 1H), 7.41 (d, J = 7.8 Hz, 1H), 7.30 (d, J = 7.8 Hz, 1H), 6.96 (d, J = 2.6 Hz, 1H), 6.93 (d, J = 8.2 Hz, 1H), | 335.3 |
| | 7-((1-(2,3- | | 6.55 (d, J = 7.4 Hz, | |
| | dichlorophenyl)ethyl) | | 1H), 6.49 (dd, J = 8.0, | |
| | amino)-3,4- | | 2.6 Hz, 1H), 4.81 (p, | |
| | dihydroisoquinolin- | | J = 6.6 Hz, 1H), 3.27- | |
| | 1(2H)-one | | 3.19 (m, 2H), 2.65 (d, | |
| | | | J = 6.4 Hz, 2H), 1.42 (d, | |
| | | | J = 6.8 Hz, 3H). | |
| Example 101 Method 2 Mixture of Stereoisomers | 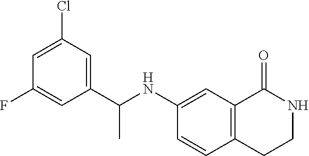 [SMILES: CC(Nc1ccc2c(c1)C(=O)NCC2)c1cc(F)cc(Cl)c1] | White solid, yield: 8.5% | ¹H NMR (400 MHz, DMSO-d₆) δ 7.72 (s, 1H), 7.32 (s, 1H), 7.22 (d, J = 8.4 Hz, 2H), 7.02 (d, J = 2.6 Hz, 1H), 6.94 (d, J = 8.2 Hz, 1H), 6.62 (dd, J = 8.2, 2.6 Hz, 1H), 6.30 (d, J = 7.4 Hz, 1H), 4.55 (p, J = 6.8 Hz, | 319.1 |
| | 7-((1-(3-chloro-5- | | 1H), 3.25 (td, J = 6.6, | |
| | fluorophenyl)ethyl) | | 2.6 Hz, 2H), 2.67 (t, J = | |
| | amino)-3,4- | | 6.6 Hz, 2H), 1.39 (d, | |
| | dihydroisoquinolin- | | J = 6.6 Hz, 3H). | |
| | 1(2H)-one | | | |
| Example 102 Method 2 Mixture of Stereoisomers | 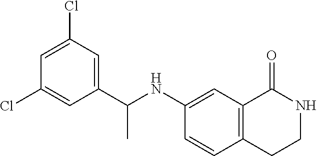 [SMILES: CC(Nc1ccc2c(c1)C(=O)NCC2)c1cc(Cl)cc(Cl)c1] | White solid, yield: 10.7% | ¹H NMR (400 MHz, DMSO-d₆) δ 7.72 (s, 1H), 7.44 (d, J = 2.0 Hz, 2H), 7.41 (t, J = 2.0 Hz, 1H), 7.02 (d, J = 2.6 Hz, 1H), 6.94 (d, J = 8.2 Hz, 1H), 6.62 (dd, J = 8.2, 2.6 Hz, 1H), 6.32 (d, J = 7.8 Hz, 1H), 4.56 (p, J = 7.0 | 335.2 |
| | 7-((1-(3,5- | | Hz, 1H), 3.25 (td, J = | |
| | dichlorophenyl)ethyl) | | 6.6, 2.8 Hz, 2H), 2.67 | |
| | amino)-3,4- | | (t, J = 6.6 Hz, 2H), 1.39 | |
| | dihydroisoquinolin- | | (d, J = 6.8 Hz, 3H). | |
| | 1(2H)-one | | | |
| Example 103 Method 2 Mixture of Stereoisomers | 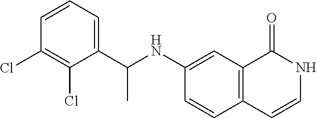 [SMILES: CC(Nc1ccc2cc[nH]c(=O)c2c1)c1cccc(Cl)c1Cl] | White solid, yield: 2.54% | ¹H NMR (400 MHz, DMSO-d₆) δ 10.87 (d, J = 5.6 Hz, 1H), 7.50 (dd, J = 7.8, 1.6 Hz, 1H), 7.42 (dd, J = 7.8, 1.6 Hz, 1H), 7.35 (d, J = 8.4 Hz, 1H), 7.29 (t, J = 7.8 Hz, 1H), 7.02-6.96 | 333.2 |
| | 7-((1-(2,3- | | (m, 2H), 6.87 (d, J = | |
| | dichlorophenyl)ethyl) | | 7.0 Hz, 1H), 6.80 (dd, J = | |
| | amino)isoquinolin- | | 7.0, 5.6 Hz, 1H), 6.32 | |
| | 1(2H)-one | | (dd, J = 7.0, 1.2 Hz, | |
| | | | 1H), 4.96-4.83 (m, | |
| | | | 1H), 1.46 (d, J = 6.6 | |
| | | | Hz, 3H). | |
| Example 104 Method 2 Mixture of Stereoisomers | 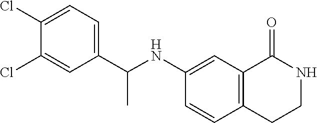 [SMILES: CC(Nc1ccc2c(c1)C(=O)NCC2)c1ccc(Cl)c(Cl)c1] | White solid, yield: 3.6% | ¹H NMR (400 MHz, DMSO-d₆) δ 7.71 (s, 1H), 7.62 (s, 1H), 7.55 (d, J = 8.2 Hz, 1H), 7.36 (d, J = 2.0 Hz, 1H), 7.01 (d, J = 2.6 Hz, 1H), 6.93 (d, J = 8.2 Hz, 1H), 6.61 (d, | 335.1 |
| | 7-((1-(3,4- | | J = 2.6 Hz, 1H), 6.31 (d, | |
| | dichlorophenyl)ethyl) | | J = 7.4 Hz, 1H), 4.53 | |
| | amino)-3,4- | | (p, J = 6.8 Hz, 1H), | |
| | dihydroisoquinolin- | | 3.28-3.21 (m, 2H), | |
| | 1(2H)-one | | 2.66 (d, J = 6.4 Hz, | |
| | | | 2H), 1.39 (d, J = 6.6 | |
| | | | Hz, 3H). | |
| Example 105 Method 2 Mixture of Stereoisomers | 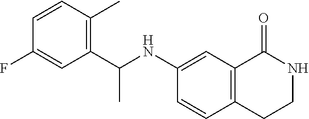 [SMILES: Cc1ccc(F)cc1C(C)Nc1ccc2c(c1)C(=O)NCC2] | White solid, yield: 5.1% | ¹H NMR (400 MHz, DMSO-d₆) δ 7.69 (s, 1H), 7.17 (dd, J = 8.4, 6.0 Hz, 1H), 7.09 (dd, J = 10.6, 2.8 Hz, 1H), 6.98 (d, J = 2.6 Hz, 1H), 6.96-6.79 (m, 2H), 6.51 (dd, J = 8.2, | 299.1 |
| | 7-((1-(5-fluoro-2- | | 2.6 Hz, 1H), 6.31 (d, | |
| | methylphenyl)ethyl) | | J = 7.2 Hz, 1H), 4.65- | |
| | amino)-3,4- | | 4.51 (m, 1H), 3.24 (td, | |
| | dihydroisoquinolin- | | J = 6.6, 2.8 Hz, 2H), 2.65 | |
| | 1(2H)-one | | (t, J = 6.6 Hz, 2H), 2.38 | |
| | | | (s, 3H), 1.35 (d, J = 6.6 | |
| | | | Hz, 3H). | |
| Example 106 Method 2 Mixture of Stereoisomers | 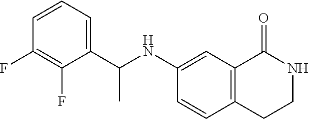 [SMILES: CC(Nc1ccc2c(c1)C(=O)NCC2)c1cccc(F)c1F] | White solid, yield: 4.9% | ¹H NMR (400 MHz, DMSO-d₆) δ 7.71 (s, 1H), 7.30-7.08 (m, 3H), 7.02 (d, J = 2.6 Hz, 1H), 6.94 (d, J = 8.2 Hz, 1H), 6.60 (dd, J = 8.2, 2.6 Hz, 1H), 6.35 (d, J = 7.6 Hz, 1H), | 303.3 |
| | 7-((1-(2,3- | | 4.78 (p, J = 7.0 Hz, | |
| | difluorophenyl)ethyl) | | 1H), 3.24 (td, J = 6.6, | |
| | amino)-3,4- | | 2.6 Hz, 2H), 2.71-2.61 | |
| | dihydroisoquinolin- | | (m, 2H), 1.45 (d, J = | |
| | 1(2H)-one | | 6.6 Hz, 3H). | |
| Example 107 Method 2 Mixture of Stereoisomers | 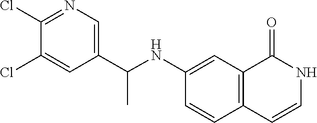 [SMILES: CC(Nc1ccc2cc[nH]c(=O)c2c1)c1cnc(Cl)c(Cl)c1] | White solid, yield: 1.9% | ¹H NMR (400 MHz, DMSO-d₆) δ 10.91 (d, J = 5.6 Hz, 1H), 8.46 (d, J = 2.0 Hz, 1H), 8.14 (d, J = 2.0 Hz, 1H), 7.37 (d, J = 8.4 Hz, 1H), 7.13-7.05 (m, 2H), 6.83 (dd, J = 7.0, | 334.2 |
| | 7-((1-(5,6- | | 5.6 Hz, 1H), 6.65 (d, | |
| | dichloropyridin-3- | | J = 7.4 Hz, 1H), 6.36- | |
| | yl)ethyl)amino) | | 6.32 (m, 1H), 4.76 (p, | |
| | isoquinolin-1(2H)-one | | J = 6.8 Hz, 1H), 1.48 (d, | |
| | | | J = 6.8 Hz, 3H). | |
| Example 108 Method 2 Mixture of Stereoisomers | 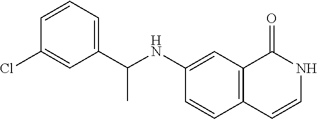 [SMILES: CC(Nc1ccc2cc[nH]c(=O)c2c1)c1cccc(Cl)c1] | White solid, yield: 7.5% | ¹H NMR (400 MHz, DMSO-d₆) δ 10.86 (d, J = 5.6 Hz, 1H), 7.46- 7.42 (m, 1H), 7.39- 7.31 (m, 3H), 7.26-7.21 (m, 1H), 7.10-7.02 (m, 2H), 6.80 (dd, J = 7.0, 5.6 Hz, 1H), 6.64 | 299.2 |
| | 7-((1-(3- | | (d, J = 7.2 Hz, 1H), | |
| | chlorophenyl)ethyl) | | 6.32 (d, J = 7.0 Hz, | |
| | amino)isoquinolin- | | 1H), 4.61 (p, J = 6.8 | |
| | 1(2H)-one | | Hz, 1H), 1.44 (d, J = | |
| | | | 6.6 Hz, 3H). | |
| Example 109 Method 2 Mixture of Stereoisomers | 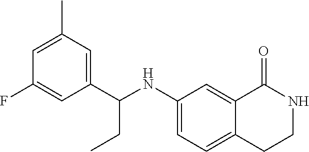 [SMILES: CCC(Nc1ccc2c(c1)C(=O)NCC2)c1cc(C)cc(F)c1] | White solid, yield: 12.8% | ¹H NMR (400 MHz, DMSO-d₆) δ 7.69 (d, J = 2.8 Hz, 1H), 7.05 (d, J = 2.4 Hz, 1H), 7.02 (d, J = 2.0 Hz, 1H), 6.95 (d, J = 10.0, 1H), 6.91 (d, J = 8.0 Hz, 1H), 6.81 (d, J = 10.0, 1H), 6.66-6.59 (m, 1H), 6.19 (d, J = 7.6 | 313.1 |
| | 7-((1-(3-fluoro-5- | | Hz, 1H), 4.23 (q, J = | |
| | methylphenyl)propyl) | | 7.2 Hz, 1H), 3.29-3.17 | |
| | amino)-3,4- | | (m, 2H), 2.65 (t, J = 6.4 | |
| | dihydroisoquinolin- | | Hz, 2H), 2.28 (s, 3H), | |
| | 1(2H)-one | | 1.81-1.57 (m, 2H), | |
| | | | 0.90 (t, J = 7.2 Hz, 3H). | |
| Example 110 Method 2 Mixture of Stereoisomers | 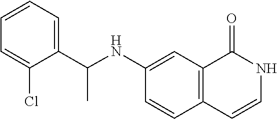 [SMILES: CC(Nc1ccc2cc[nH]c(=O)c2c1)c1ccccc1Cl] | White solid, yield: 4.02% | ¹H NMR (400 MHz, DMSO-d₆) δ 10.85 (d, J = 4.8 Hz, 1H), 7.48- 7.41 (m, 2H), 7.33 (d, J = 8.4 Hz, 1H), 7.29- 7.19 (m, 2H), 7.04- 6.96 (m, 2H), 6.83- 6.75 (m, 2H), 6.31 (d, | 299.2 |
| | 7-((1-(2- | | J = 6.8 Hz, 1H), 4.87 (p, | |
| | chlorophenyl)ethyl) | | J = 6.8 Hz, 1H), 1.45 | |
| | amino)isoquinolin- | | (d, J = 6.8 Hz, 3H). | |
| | 1(2H)-one | | | |
| Example 111 Method 2 single unknown stereoisomer | 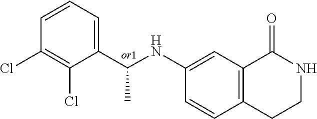 [SMILES: C[C@@H](Nc1ccc2c(c1)C(=O)NCC2)c1cccc(Cl)c1Cl] | White solid, yield: 6.5% | ¹H NMR (400 MHz, DMSO-d₆) δ 7.75-7.69 (m, 1H), 7.49 (dd, J = 7.8, 1.6 Hz, 1H), 7.40 (dd, J = 7.8, 1.7 Hz, 1H), 7.33-7.26 (m, 1H), 6.96 (d, J = 2.6 Hz, 1H), 6.93 (d, J = 8.2 | 335.1 Note: CHIRAL Cellulose SB, 2 cm * 25 cm, 5 um, MTBE: |
| | rel-(R)-7-((1-(2,3- | | Hz, 1H), 6.55 (d, J = | (MeOH: |
| | dichlorophenyl)ethyl) | | 7.3 Hz, 1H), 6.48 (dd, | DCM) = |
| | amino)-3,4- | | J = 8.2, 2.6 Hz, 1H), 4.81 | 80:20 |
| | dihydroisoquinolin- | | (p, J = 6.7 Hz, 1H), | Rt = 5.480 |
| | 1(2H)-one | | 3.23 (td, J = 6.7, 2.7 | min |
| | | | Hz, 2H), 2.65 (t, J = 6.6 | |
| | | | Hz, 2H), 1.41 (d, J = | |
| | | | 6.7 Hz, 3H). | |
| Example 112 Method 2 single unknown stereoisomer | 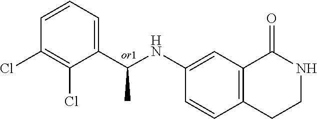 [SMILES: C[C@H](Nc1ccc2c(c1)C(=O)NCC2)c1cccc(Cl)c1Cl] | White solid, yield: 6.1% | ¹H NMR (400 MHz, DMSO-d₆) δ 7.75-7.69 (m, 1H), 7.49 (dd, J = 7.8, 1.6 Hz, 1H), 7.40 (dd, J = 7.8, 1.7 Hz, 1H), 7.33-7.26 (m, 1H), 6.96 (d, J = 2.6 Hz, 1H), 6.93 (d, J = 8.2 | 335.1 Note: CHIRAL Cellulose SB, 2 cm * 25 cm, 5 um, MTBE: |
| | rel-(R)-7-((1-(2,3- | | Hz, 1H), 6.55 (d, J = | (MeOH: |
| | dichlorophenyl)ethyl) | | 7.3 Hz, 1H), 6.48 (dd, J = | DCM) = |
| | amino)-3,4- | | 8.2, 2.6 Hz, 1H), 4.81 | 80:20 |
| | dihydroisoquinolin- | | (p, J = 6.7 Hz, 1H), | Rt = 6.191 |
| | 1(2H)-one | | 3.23 (td, J = 6.7, 2.7 | min |
| | | | Hz, 2H), 2.65 (t, J = 6.6 | |
| | | | Hz, 2H), 1.41 (d, J = | |
| | | | 6.7 Hz, 3H). | |
| Example 113 Method 2 Mixture of Stereoisomers | 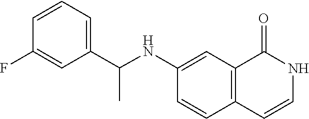 [SMILES: CC(Nc1ccc2cc[nH]c(=O)c2c1)c1cccc(F)c1] | White solid, yield: 10.9% | 1H NMR (400 MHz, DMSO-d₆) δ 10.85 (d, J = 5.6 Hz, 1H), 7.38- 7.29 (m, 2H), 7.24 (d, J = 8.0 Hz, 1H), 7.19 (dt, J = 10.4, 2.4 Hz, 1H), 7.08 (d, J = 2.4 Hz, 1H), 7.05 (dd, J = 8.4, | 283.2 |
| | 7-((1-(3- | | 2.4 Hz, 1H), 7.03-6.97 | |
| | fluorophenyl)ethyl) | | (m, 1H), 6.79 (dd, J = | |
| | amino)isoquinolin- | | 7.2, 5.6 Hz, 1H), 6.62 | |
| | 1(2H)-one | | (d, J = 7.2 Hz, 1H), | |
| | | | 6.32 (dd, J = 7.2, 1.2 | |
| | | | Hz, 1H), 4.61 (p, J = | |
| | | | 6.8 Hz, 1H), 1.45 (d, | |
| | | | J = 6.8 Hz, 3H). | |
| Example 114 Method 2 Mixture of Stereoisomers | 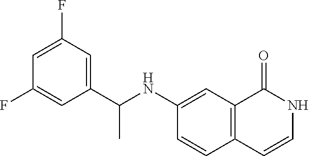 [SMILES: CC(Nc1ccc2cc[nH]c(=O)c2c1)c1cc(F)cc(F)c1] | White solid, yield: 12.9% | ¹H NMR (400 MHz, DMSO-d₆) δ 10.88 (d, J = 5.6 Hz, 1H), 7.35 (d, J = 8.4 Hz, 1H), 7.19- 6.98 (m, 5H), 6.81 (dd, J = 7.2, 5.6 Hz, 1H), 6.63 (d, J = 7.3 Hz, 1H), 6.33 (d, J = 6.4 Hz, 1H), 4.64 (p, J = 6.8 Hz, 1H), 1.44 (d, | 301.2 |
| | 7-((1-(3,5- | | J = 6.8 Hz, 3H). | |
| | difluorophenyl)ethyl) | | | |
| | amino)isoquinolin- | | | |
| | 1(2H)-one | | | |
| Example 115 Method 2 Mixture of Stereoisomers | 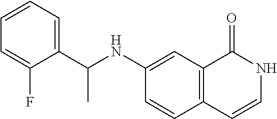 [SMILES: CC(Nc1ccc2cc[nH]c(=O)c2c1)c1ccccc1F] | White solid, yield: 10.8% | ¹H NMR (400 MHz, DMSO-d₆) δ 10.87 (d, J = 5.6 Hz, 1H), δ 7.38 (td, J = 7.6, 1.6 Hz, 1H), 7.34 (d, J = 8.4 Hz, 1H), 7.28-7.15 (m, 2H), 7.11 (td, J = 7.2, 1.3 Hz, 1H), 7.05 | 283.2 |
| | 7-((1-(2- | | (dd, J = 10.4, 2.0 Hz, | |
| | fluorophenyl)ethyl) | | 2H), 6.80 (dd, J = 6.8, | |
| | amino)isoquinolin- | | 5.6 Hz, 1H), 6.65 (d, | |
| | 1(2H)-one | | J = 7.2 Hz, 1H), 6.32 (d, | |
| | | | J = 6.8 Hz, 1H), 4.83 | |
| | | | (p, J = 6.8 Hz, 1H), | |
| | | | 1.48 (d, J = 6.8 Hz, | |
| | | | 3H). | |
| Example 116 Method 2 Mixture of Stereoisomers | 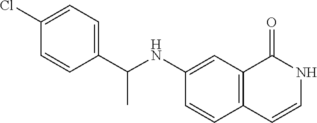 [SMILES: CC(Nc1ccc2cc[nH]c(=O)c2c1)c1ccc(Cl)cc1] | White solid, yield: 13.2% | ¹H NMR (400 MHz, DMSO-d₆) δ 10.85 (d, J = 5.6 Hz, 1H), 7.45- 7.29 (m, 5H), 7.09-7.01 (m, 2H), 6.79 (t, J = 6.0 Hz, 1H), 6.63 (d, J = 6.8 Hz, 1H), 6.31 (d, J = 6.8 Hz, 1H), 4.58 (p, | 299.1 |
| | 7-((1-(4- | | J = 6.8 Hz, 1H), 1.43 | |
| | chlorophenyl)ethyl) | | (d, J = 6.8 Hz, 3H). | |
| | amino)isoquinolin- | | | |
| | 1(2H)-one | | | |
| Example 117 Method 2 Mixture of Stereoisomers | 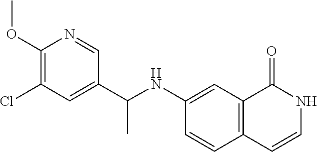 [SMILES: COc1ncc(C(C)Nc2ccc3cc[nH]c(=O)c3c2)cc1Cl] | White solid, yield: 9.2% | ¹H NMR (400 MHz, DMSO-d₆) δ 10.89 (d, J = 5.6 Hz, 1H), 8.16 (d, J = 2.0 Hz, 1H), 7.89 (d, J = 2.0 Hz, 1H), 7.35 (d, J = 8.4 Hz, 1H), 7.12 (d, J = 2.4 Hz, 1H), 7.07 (dd, J = 8.6, 2.6 Hz, 1H), 6.81 (dd, J = 7.2, 5.6 Hz, 1H), 6.57 | 330.1 |
| | 7-((1-(5-chloro-6- | | (d, J = 7.6 Hz, 1H), | |
| | methoxypyridin-3- | | 6.33 (d, J = 6.8 Hz, | |
| | yl)ethyl)amino) | | 1H), 4.65 (p, J = 6.8 | |
| | isoquinolin-1(2H)-one | | Hz, 1H), 3.89 (s, 3H), | |
| | | | 1.46 (d, J = 6.8 Hz, | |
| | | | 3H). | |
| Example 118 Method 2 Mixture of Stereoisomers | 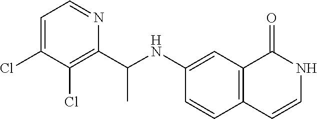 [SMILES: CC(Nc1ccc2cc[nH]c(=O)c2c1)c1nccc(Cl)c1Cl] | White solid, yield: 8.1% | ¹H NMR (400 MHz, DMSO-d₆) δ 10.87 (d, J = 5.6 Hz, 1H), 8.44 (d, J = 5.2 Hz, 1H), 7.63 (d, J = 5.2 Hz, 1H), 7.34 (d, J = 8.4 Hz, 1H), 7.12 (d, J = 2.4 Hz, 1H), 7.09 (dd, J = | 334.1 |
| | 7-((1-(3,4- | | 8.4, 2.4 Hz, 1H), 6.81 | |
| | dichloropyridin-2- | | (dd, J = 6.8, 5.6 Hz, | |
| | yl)ethyl)amino) | | 1H), 6.63 (d, J = 8.8 | |
| | isoquinolin-1(2H)-one | | Hz, 1H), 6.32 (d, J = | |
| | | | 6.8 Hz, 1H), 5.22-5.12 | |
| | | | (m, 1H), 1.48 (d, J = | |
| | | | 6.8 Hz, 3H). | |
| Example 119 Method 2 Mixture of Stereoisomers | 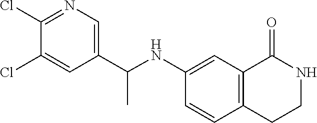 [SMILES: CC(Nc1ccc2c(c1)C(=O)NCC2)c1cnc(Cl)c(Cl)c1] | White solid, yield: 9.3% | ¹H NMR (400 MHz, DMSO-d₆) δ 8.42 (d, J = 2.0 Hz, 1H), 8.12 (d, J = 2.0 Hz, 1H), 7.74 (s, 1H), 7.04 (d, J = 2.4 Hz, 1H), 6.96 (d, J = 8.0 Hz, 1H), 6.66 (dd, J = 8.0, 2.4 Hz, 1H), 6.31 | 337.1 |
| | 7-((1-(5,6- | | (s, 1H), 4.66 (q, J = 6.4 | |
| | dichloropyridin-3- | | Hz, 1H), 3.25 (td, J = | |
| | yl)ethyl)amino)-3,4- | | 6.4, 2.8 Hz, 3H), 2.67 | |
| | dihydroisoquinolin- | | (t, J = 6.4 Hz, 3H), 1.44 | |
| | 1(2H)-one | | (d, J = 6.8 Hz, 3H). | |
| Example 120 Method 2 Mixture of Stereoisomers | 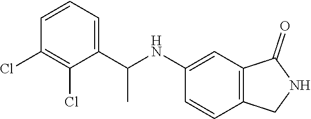 [SMILES: CC(Nc1ccc2c(c1)C(=O)NC2)c1cccc(Cl)c1Cl] | White solid, yield: 11.2% | ¹H NMR (400 MHz, DMSO-d₆) δ 8.32 (s, 1H), 7.50 (dd, J = 7.9, 1.6 Hz, 1H), 7.41 (dd, J = 7.9, 1.6 Hz, 1H), 7.29 (t, J = 7.9 Hz, 1H), 7.20 (d, J = 8.2 Hz, 1H), 6.77-6.68 (m, 2H), | 321.1 |
| | 6-((1-(2,3- | | 6.51 (d, J = 2.2 Hz, | |
| | dichlorophenyl)ethyl) | | 1H), 4.84 (p, J = 6.7 | |
| | amino)isoindolin-1- | | Hz, 1H), 4.12 (s, 2H), | |
| | one | | 1.45 (d, J = 6.6 Hz, | |
| | | | 3H). | |
| Example 121 Method 2 Mixture of Stereoisomers | 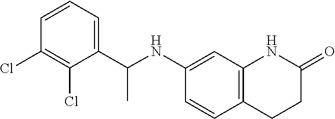 [SMILES: CC(Nc1ccc2c(c1)NC(=O)CC2)c1cccc(Cl)c1Cl] | White solid, yield: 10.4% | ¹H NMR (400 MHz, DMSO-d₆) δ 9.87 (s, 1H), 7.48 (dd, J = 8.0, 1.6 Hz, 1H), 7.39 (dd, J = 7.6, 1.6 Hz, 1H), 7.29 (t, J = 8.0 Hz, 1H), 6.76 (d, J = 8.0 Hz, 1H), 6.38 (d, J = 7.2 Hz, | 335.1 |
| | 7-((1-(2,3- | | 1H), 5.99 (d, J = 2.4 | |
| | dichlorophenyl)ethyl) | | Hz, 1H), 5.95 (dd, J = | |
| | amino)-3,4- | | 8.0, 2.4 Hz, 1H), 4.72 | |
| | dihydroquinolin- | | (t, J = 6.8 Hz, 1H), 2.62 | |
| | 2(1H)-one | | (t, J = 7.2 Hz, 2H), 2.34- | |
| | | | 2.28 (m, 2H), 1.39 (d, | |
| | | | J = 6.8 Hz, 3H). | |
| Example 122 Method 2 Mixture of Stereoisomers | 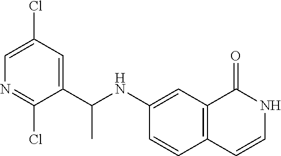 [SMILES: CC(Nc1ccc2cc[nH]c(=O)c2c1)c1cc(Cl)cnc1Cl] | White solid, yield: 1.52% | ¹H NMR (400 MHz, DMSO-d₆) δ 10.93 (d, J = 5.6 Hz, 1H), 8.40 (d, J = 2.6 Hz, 1H), 7.94 (d, J = 2.6 Hz, 1H), 7.39 (d, J = 8.4 Hz, 1H), 7.04-7.00 (m, 2H), 6.87-6.79 (m, 2H), 6.34 (d, J = 7.0 Hz, 1H), 4.80 (p, J = | 334.2 |
| | 7-((1-(2,5- | | 6.8 Hz, 1H), 1.49 (d, | |
| | dichloropyridin-3- | | J = 6.8 Hz, 3H). | |
| | yl)ethyl)amino) | | | |
| | isoquinolin-1(2H)-one | | | |
| Example 123 Method 2 Mixture of Stereoisomers | 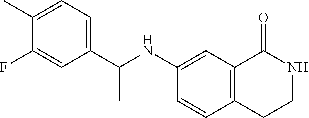 [SMILES: Cc1ccc(C(C)Nc2ccc3c(c2)C(=O)NCC3)cc1F] | White solid, yield: 9.9% | ¹H NMR (400 MHz, DMSO-d₆) δ 7.69 (s, 1H), 7.18 (t, J = 8.0 Hz, 1H), 7.13-7.05 (m, 2H), 7.01 (d, J = 2.4 Hz, 1H), 6.91 (d, J = 8.0 Hz, 1H), 6.60 (dd, J = 8.0, 2.4 Hz, 1H), 6.22 | 299.2 |
| | 7-((1-(3-fluoro-4- | | (d, J = 7.2 Hz, 1H), | |
| | methylphenyl)ethyl) | | 4.46 (p, J = 6.8 Hz, | |
| | amino)-3,4- | | 1H), 3.24 (td, J = 6.4, | |
| | dihydroisoquinolin- | | 2.8 Hz, 2H), 2.68-2.62 | |
| | 1(2H)-one | | (m, 2H), 2.16 (d, J = | |
| | | | 1.6 Hz, 3H), 1.38 (d, | |
| | | | J = 6.8 Hz, 3H). | |
| Example 124 Method 2 | 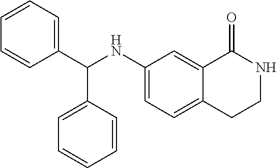 [SMILES: O=C1NCCc2ccc(NC(c3ccccc3)c3ccccc3)cc21] | white solid, yield: 1.73% | ¹H NMR (400 MHz, DMSO-d₆) δ 7.70 (d, J = 3.2 Hz, 1H), 7.43- 7.37 (m, 4H), 7.34- 7.27 (m, 4H), 7.24- 7.15 (m, 3H), 6.94 (d, J = 8.0 Hz, 1H), 6.80 (dd, J = 8.0, 2.4 Hz, 1H), 6.52 (d, J = 7.2 Hz, 1H), 5.65 (d, J = 7.2 Hz, 1H), 3.28-3.22 | 329.3 |
| | 7-(benzhydrylamino)- | | (m, 2H), 2.67 (t, J = 6.4 | |
| | 3,4-dihydroisoquinolin- | | Hz, 2H). | |
| | 1(2H)-one | | | |
| Example 125 Method 2 Mixture of Stereoisomers | 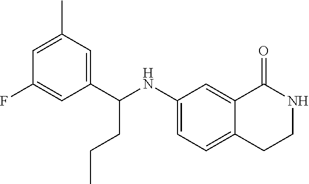 [SMILES: CCCC(Nc1ccc2c(c1)C(=O)NCC2)c1cc(C)cc(F)c1] | white solid, yield: 24.7% | ¹H NMR (400 MHz, DMSO-d₆) δ 7.70 (d, J = 2.8 Hz, 1H), 7.05 (d, J = 2.4 Hz, 1H), 7.02 (s, 1H), 6.95 (d, J = 10.1, 1H), 6.90 (d, J = 8.0 Hz, 1H), 6.81 (d, J = 10.0 Hz, 1H), 6.63 (dd, J = 8.0, 2.4 Hz, 1H), 6.20 (d, J = 8.0 Hz, 1H), 4.39-4.24 (m, 1H), 3.29-3.17 (m, | 327.2 |
| | 7-((1-(3-fluoro-5- | | 2H), 2.65 (t, J = 6.4 Hz, | |
| | methylphenyl)butyl) | | 2H), 2.27 (s, 3H), 1.77- | |
| | amino)-3,4- | | 1.66 (m, 1H), 1.64- | |
| | dihydroisoquinolin- | | 1.53 (m, 1H), 1.48- | |
| | 1(2H)-one | | 1.35 (m, 1H), 1.34- | |
| | | | 1.21 (m, 1H), 0.88 (t, | |
| | | | J = 7.2 Hz, 3H). | |
| Example 126 Method 2 Mixture of Stereoisomers | 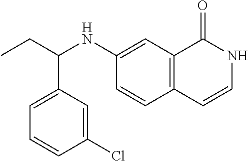 [SMILES: CCC(Nc1ccc2cc[nH]c(=O)c2c1)c1cccc(Cl)c1] | White solid, yield: 10.5% | ¹H NMR (400 MHz, Methanol-d₄) δ 7.40 (d, J = 2.0 Hz, 1H), 7.37 (d, J = 8.8 Hz, 1H), 7.33 (dt, J = 7.6, 1.2 Hz, 1H), 7.26 (t, J = 7.6 Hz, 1H), 7.22 (d, J = 2.4 Hz, 1H), 7.17 (dt, J = 7.6, 1.6 Hz, 1H), 7.12 (dd, J = 8.8, 2.4 Hz, | 313.2 |
| | 7-((1-(3- | | 1H), 6.86 (d, J = 7.2 | |
| | chlorophenyl)propyl) | | Hz, 1H), 6.51 (d, J = | |
| | amino)isoquinolin- | | 7.2 Hz, 1H), 4.36 (dd, | |
| | 1(2H)-one | | J = 7.6, 6.2 Hz, 1H), | |
| | | | 1.96-1.72 (m, 2H), 1.00 | |
| | | | (t, J = 7.6 Hz, 3H). | |
| Example 127 Method 2 Mixture of Stereoisomers | 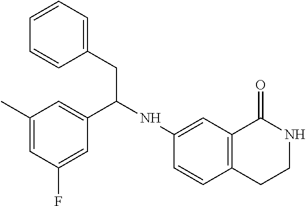 [SMILES: Cc1cc(F)cc(C(Cc2ccccc2)Nc2ccc3c(c2)C(=O)NCC3)c1] | White solid, yield: 5.16% | ¹H NMR (400 MHz, Methanol-d₄) δ 7.33- 7.07 (m, 6H), 6.98- 6.87 (m, 2H), 6.84- 6.78 (m, 1H), 6.76- 6.61 (m, 2H), 4.65- 4.53 (m, 1H), 3.44- 3.34 (m, 2H), 3.09 (dd, J = 13.4, 7.7 Hz, 1H), 2.97 (dd, J = 13.6, 6.7 Hz, 1H), 2.74 (td, J = 6.6, 2.7 Hz, 2H), 2.25 (s, 3H). | 375.2 |
| | 7-((1-(3-fluoro-5- | | | |
| | methylphenyl)-2- | | | |
| | phenylethyl)amino)- | | | |
| | 3,4- | | | |
| | dihydroisoquinolin- | | | |
| | 1(2H)-one | | | |
| Example 128 Method 2 Mixture of Stereoisomers | 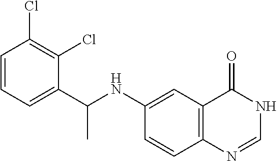 [SMILES: CC(Nc1ccc2nc[nH]c(=O)c2c1)c1cccc(Cl)c1Cl] | White solid, yield: 11.2% | ¹H NMR (400 MHz, DMSO-d₆) δ 11.86 (s, 1H), 7.74 (s, 1H), 7.51 (dd, J = 7.8, 1.6 Hz, 1H), 7.43-7.38 (m, 2H), 7.30 (t, J = 7.8 Hz, 1H), 7.09 (dd, J = 8.8, 2.8 Hz, 1H), 7.01 (d, J = 7.0 Hz, 1H), 6.82 (d, J = 2.8 Hz, 1H), 4.89 (t, | 334.1 |
| | 6-((1-(2,3- | | J = 6.8 Hz, 1H), 1.46 | |
| | dichlorophenyl)ethyl) | | (d, J = 6.6 Hz, 3H). | |
| | amino)quinazolin- | | | |
| | 4(3H)-one | | | |
| Example 129 Method 2 Mixture of Stereoisomers | 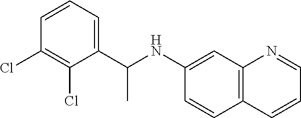 [SMILES: CC(Nc1ccc2cccnc2c1)c1cccc(Cl)c1Cl] | White solid, yield: 18.8% | ¹H NMR (400 MHz, DMSO-d₆) δ 8.57 (d, J = 3.6 Hz, 1H), 8.13 (d, J = 7.8 Hz, 1H), 7.70 (d, J = 8.8 Hz, 1H), 7.52 (dd, J = 8.0, 1.4 Hz, 1H), 7.44 (d, J = 7.6 Hz, 1H), 7.39 (br s, | 317.1 |
| | N-(1-(2,3- | | 1H), 7.30 (t, J = 8.0 Hz, | |
| | dichlorophenyl)ethyl) | | 1H), 7.20-7.12 (m, | |
| | quinolin-7-amine | | 2H), 6.39 (s, 1H), 4.94 | |
| | | | (p, J = 6.4 Hz, 1H), | |
| | | | 1.51 (d, J = 6.8 Hz, | |
| | | | 3H). | |
| Example 130 Method 2 Mixture of Stereoisomers | 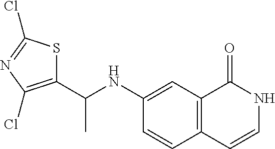 [SMILES: CC(Nc1ccc2cc[nH]c(=O)c2c1)c1sc(Cl)nc1Cl] | White solid, yield: 5.7% | ¹H NMR (400 MHz, DMSO-d₆) δ 10.96 (d, J = 5.6 Hz, 1H), 7.42 (d, J = 8.4 Hz, 1H), 7.14 (d, J = 2.4 Hz, 1H), 7.04 (dd, J = 8.4, 2.4 Hz, 1H), 6.87 (dd, J = 7.2, 5.6 Hz, 1H), 6.83 (d, J = 5.2 Hz, 1H), 6.37 (d, J = 6.8 Hz, | 340.1 |
| | 7-((1-(2,4- | | 1H), 4.82-4.71 (m, | |
| | dichlorothiazol-5- | | 1H), 1.56 (d, J = 6.8 | |
| | yl)ethyl)amino) | | Hz, 3H). | |
| | isoquinolin-1(2H)-one | | | |
| Example 131 Method 2 Mixture of Stereoisomers | 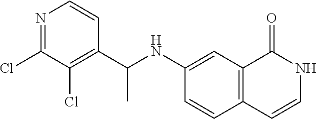 [SMILES: CC(Nc1ccc2cc[nH]c(=O)c2c1)c1ccnc(Cl)c1Cl] | White solid, yield: 7.3% | ¹H NMR (400 MHz, DMSO-d₆) δ 10.90 (d, J = 5.6 Hz, 1H), 8.29 (d, J = 4.8 Hz, 1H), 7.45 (d, J = 4.8 Hz, 1H), 7.38 (d, J = 8.4 Hz, 1H), 7.00 (dd, J = 8.4, 2.4 Hz, 1H), 6.97-6.91 | 334.2 |
| | 7-((1-(2,3- | | (m, 2H), 6.82 (dd, J = | |
| | dichloropyridin-4- | | 7.2, 5.6 Hz, 1H), 6.33 | |
| | yl)ethyl)amino) | | (d, J = 6.8 Hz, 1H), | |
| | isoquinolin-1(2H)-one | | 4.88 (p, J = 6.8 Hz, | |
| | | | 1H), 1.48 (d, J = 6.8 | |
| | | | Hz, 3H). | |
| Example 132 Method 2 Mixture of Stereoisomers | 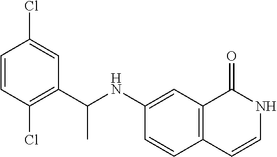 [SMILES: CC(Nc1ccc2cc[nH]c(=O)c2c1)c1cc(Cl)ccc1Cl] | White solid, yield: 9.3% | ¹H NMR (400 MHz, DMSO-d₆) δ 10.89 (d, J = 5.6 Hz, 1H), 7.51 (d, J = 8.5 Hz, 1H), 7.47 (d, J = 2.6 Hz, 1H), 7.37 (d, J = 9.1 Hz, 1H), 7.31 (dd, J = 8.5, 2.6 Hz, 1H), 7.00 (d, J = 7.6 Hz, 2H), 6.87- 6.76 (m, 2H), 6.33 (dd, | 333.1 |
| | 7-((1-(2,5- | | J = 7.1, 1.3 Hz, 1H), | |
| | dichlorophenyl)ethyl) | | 4.84 (p, J = 6.7 Hz, | |
| | amino)isoquinolin- | | 1H), 1.45 (d, J = 6.7 | |
| | 1(2H)-one | | Hz, 3H). | |
| Example 133 Method 2 Mixture of Stereoisomers | 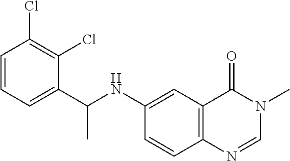 [SMILES: CC(Nc1ccc2ncn(C)c(=O)c2c1)c1cccc(Cl)c1Cl] | White solid, yield: 56.2% | ¹H NMR (400 MHz, DMSO-d₆) δ 8.03 (s, 1H), 7.50 (dd, J = 8.0, 1.2 Hz, 1H), 7.44-7.37 (m, 2H), 7.29 (t, J = 8.0 Hz, 1H), 7.09 (dd, J = 8.8, 2.8 Hz, 1H), 7.04 (d, J = 6.8 Hz, 1H), 6.84 (d, J = 2.8 Hz, 1H), 4.89 (p, J = 6.8 | 348.2 |
| | 6-((1-(2,3- | | Hz, 1H), 3.39 (s, 3H), | |
| | dichlorophenyl)ethyl) | | 1.47 (d, J = 6.4 Hz, | |
| | amino)-3- | | 3H). | |
| | methylquinazolin- | | | |
| | 4(3H)-one | | | |
| Example 134 Method 2 Mixture of Stereoisomers | 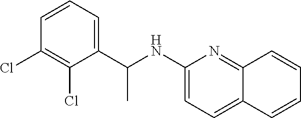 [SMILES: CC(Nc1ccc2ccccc2n1)c1cccc(Cl)c1Cl] | Off- white solid, yield: 78.1% | ¹H NMR (400 MHz, DMSO-d₆) δ 7.86 (d, J = 8.8 Hz, 1H), 7.74 (d, J = 7.4 Hz, 1H), 7.59 (dd, J = 8.0, 1.6 Hz, 1H), 7.47 (td, J = 7.6, 1.6 Hz, 2H), 7.41 (ddd, J = 8.4, 6.8, 1.6 Hz, | 317.1 |
| | N-(1-(2,3- | | 1H), 7.35-7.25 (m, | |
| | dichlorophenyl)ethyl) | | 2H), 7.12 (ddd, J = 8.0, | |
| | quinolin-2-amine | | 6.8, 1.2 Hz, 1H), 6.84 | |
| | | | (d, J = 8.8 Hz, 1H), | |
| | | | 5.61 (p, J = 6.8 Hz, | |
| | | | 1H), 1.46 (d, J = 6.8 | |
| | | | Hz, 3H) | |
| Example 135 Method 2 Mixture of Stereoisomers | 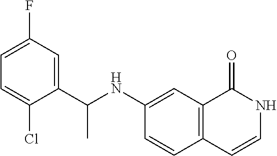 [SMILES: CC(Nc1ccc2cc[nH]c(=O)c2c1)c1cc(F)ccc1Cl] | White solid, yield: 3.84% | ¹H NMR (400 MHz, DMSO-d₆) δ 10.89 (d, J = 5.6 Hz, 1H), 7.52 (dd, J = 8.8, 5.2 Hz, 1H), 7.37 (d, J = 9.2 Hz, 1H), 7.24 (dd, J = 9.6, 3.2 Hz, 1H), 7.11 (td, J = 8.4, 3.2 Hz, 1H), 7.06- 6.96 (m, 2H), 6.88- 6.71 (m, 2H), 6.33 (d, | 316.7 |
| | 7-((1-(2-chloro-5- | | J = 7.0 Hz, 1H), 4.84 (m, | |
| | fluorophenyl)ethyl) | | 1H), 1.46 (d, J = 6.8 | |
| | amino)isoquinolin- | | Hz, 3H). | |
| | 1(2H)-one | | | |
| Example 136 Method 2 Mixture of Stereoisomers | 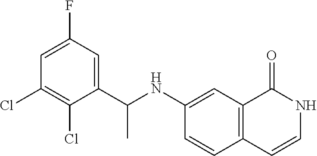 [SMILES: CC(Nc1ccc2cc[nH]c(=O)c2c1)c1cc(F)cc(Cl)c1Cl] | White solid, yield: 1.73% | ¹H NMR (400 MHz, DMSO-d₆) δ 10.91 (d, J = 5.6 Hz, 1H), 7.58 (dd, J = 8.0, 3.2 Hz, 1H), 7.38 (d, J = 8.4 Hz, 1H), 7.27 (dd, J = 9.2, 3.2 Hz, 1H), 7.05-6.95 (m, 2H), 6.92-6.77 (m, 2H), 6.34 (d, J = 7.2 Hz, 1H), 4.90 (p, | 351.2 |
| | 7-((1-(2,3-dichloro- | | J = 6.8 Hz, 1H), 1.46 (d, | |
| | 5- | | J = 6.8 Hz, 3H). | |
| | fluorophenyl)ethyl) | | | |
| | amino)isoquinolin- | | | |
| | 1(2H)-one | | | |
| Example 137 Method 2 Mixture of Stereoisomers | 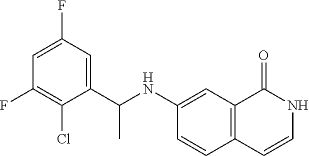 [SMILES: CC(Nc1ccc2cc[nH]c(=O)c2c1)c1cc(F)cc(F)c1Cl] | White solid, yield: 2.73% | ¹H NMR (400 MHz, DMSO-d₆) δ 10.90 (d, J = 5.6 Hz, 1H), 7.39 (dd, J = 9.6, 7.2 Hz, 2H), 7.15 (dt, J = 9.6, 2.0 Hz, 1H), 7.01 (d, J = 8.0 Hz, 2H), 6.83 (t, J = 6.0 Hz, 2H), 6.34 (d, J = 7.2 Hz, 1H), 4.90 (p, J = 6.8 Hz, 1H), 1.47 | 334.7 |
| | 7-((1-(2-chloro-3,5- | | (d, J = 6.8 Hz, 3H). | |
| | difluorophenyl)ethyl) | | | |
| | amino)isoquinolin- | | | |
| | 1(2H)-one | | | |
| Example 138 Method 2 Mixture of Stereoisomers | 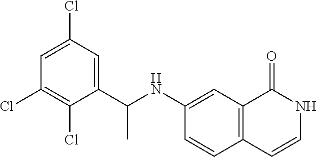 [SMILES: CC(Nc1ccc2cc[nH]c(=O)c2c1)c1cc(Cl)cc(Cl)c1Cl] | White solid, yield: 1.10% | ¹H NMR (400 MHz, DMSO-d₆) δ 10.91 (d, J = 5.6 Hz, 1H), 7.73 (d, J = 2.4 Hz, 1H), 7.47 (d, J = 2.4 Hz, 1H), 7.39 (d, J = 8.4 Hz, 1H), 7.04-6.95 (m, 2H), 6.90-6.80 (m, 2H), 6.34 (d, J = 7.0 Hz, 1H), 4.89 (p, J = | 367.6 |
| | 7-((1-(2,3,5- | | 6.8 Hz, 1H), 1.46 (d, | |
| | trichlorophenyl)ethyl) | | J = 6.8 Hz, 3H). | |
| | amino)isoquinolin- | | | |
| | 1(2H)-one | | | |
| Example 139 Method 2 Mixture of Stereoisomers | 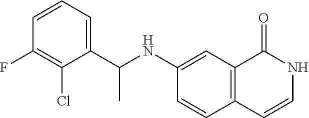 [SMILES: CC(Nc1ccc2cc[nH]c(=O)c2c1)c1cccc(F)c1Cl] | White solid, yield: 10.2% | ¹H NMR (400 MHz, DMSO-d₆) δ 10.87 (d, J = 5.6 Hz, 1H), 7.46- 7.15 (m, 4H), 7.01- 6.99 (m, 2H), 6.87- 6.76 (m, 2H), 6.32 (d, J = 7.0 Hz, 1H), 4.88 (p, J = 6.8 Hz, 1H), 1.47 | 317.1 |
| | 7-((1-(2-chloro-3- | | (d, J = 6.8 Hz, 3H). | |
| | fluorophenyl)ethyl) | | | |
| | amino)isoquinolin- | | | |
| | 1(2H)-one | | | |
| Example 140 Method 2 Mixture of Stereoisomers | 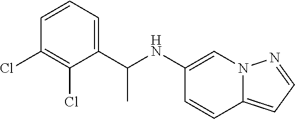 [SMILES: CC(Nc1ccc2ccnn2c1)c1cccc(Cl)c1Cl] | White solid, yield: 9.7% | ¹H NMR (400 MHz, DMSO-d₆) δ 7.61 (d, J = 2.4 Hz, 1H), 7.53 (dd, J = 7.8, 1.6 Hz, 1H), 7.51-7.42 (m, 2H), 7.33 (t, J = 7.6 Hz, 1H), 7.13-7.12 (m, 1H), 6.91 (dd, J = 9.6, 2.0 | 306.2 |
| | N-(1-(2,3- | | Hz, 1H), 6.47 (d, J = | |
| | dichlorophenyl)ethyl) | | 6.8 Hz, 1H), 6.35 (d, | |
| | pyrazolo[1,5- | | J = 2.4 Hz, 1H), 4.74 (p, | |
| | a]pyridin-6-amine | | J = 6.8 Hz, 1H), 1.46 | |
| | | | (d, J = 6.8 Hz, 3H). | |
| Example 141 Method 2 Mixture of Stereoisomers | 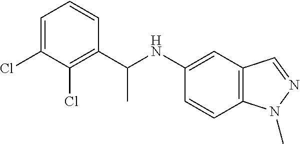 [SMILES: CC(Nc1ccc2c(cnn2C)c1)c1cccc(Cl)c1Cl] | White solid, yield: 11.1% | ¹H NMR (400 MHz, DMSO-d₆) δ 7.64 (s, 1H), 7.46 (ddd, J = 9.8, 7.8, 1.6 Hz, 2H), 7.35 (d, J = 8.8 Hz, 1H), 7.27 (t, J = 7.8 Hz, 1H), 6.91 (dd, J = 9.0, 2.0 Hz, 1H), 6.30 (d, J = 7.2 Hz, 1H), 6.21 (d, | 320.1 |
| | N-(1-(2,3- | | J = 2.0 Hz, 1H), 4.81 (p, | |
| | dichlorophenyl)ethyl)- | | J = 6.8 Hz, 1H), 3.89 (s, | |
| | 1-methyl-1H- | | 3H), 1.44 (d, J = 6.8 | |
| | indazol-5-amine | | Hz, 3H). | |
| Example 142 Method 2 Mixture of Stereoisomers | 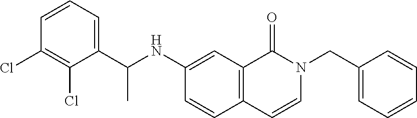 [SMILES: CC(Nc1ccc2ccn(Cc3ccccc3)c(=O)c2c1)c1cccc(Cl)c1Cl] | White solid, yield: 11.4% | ¹H NMR (400 MHz, DMSO-d₆) δ 7.50 (dd, J = 8.0, 1.6 Hz, 1H), 7.42 (dd, J = 7.8, 1.6 Hz, 1H), 7.37 (d, J = 8.5 Hz, 1H), 7.34-7.16 (m, 7H), 7.07-6.98 (m, 2H), 6.95 (d, J = | 423.2 |
| | 2-benzyl-7-((1-(2,3- | | 7.0 Hz, 1H), 6.42 (d, | |
| | dichlorophenyl)ethyl) | | J = 7.2 Hz, 1H), −5.16- | |
| | amino)isoquinolin- | | 5.01 (m, 2H), 4.90 (p, | |
| | 1(2H)-one | | J = 6.8 Hz, 1H), 1.46 (d, | |
| | | | J = 6.8 Hz, 3H). | |
| Example 143 Method 2 Mixture of Stereoisomers | 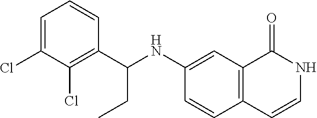 [SMILES: CCC(Nc1ccc2cc[nH]c(=O)c2c1)c1cccc(Cl)c1Cl] | White solid, yield: 12.4% | ¹H NMR (400 MHz, Methanol-d₄) δ 7.41- 7.35 (m, 3H), 7.23- 7.14 (m, 2H), 7.07 (dd, J = 8.4, 2.4 Hz, 1H), 6.86 (d, J = 7.2 Hz, 1H), 6.51 (d, J = 7.2 Hz, 1H), 4.84-4.80 | 347.3 |
| | 7-((1-(2,3- | | (m, 1H), 1.96-1.71 | |
| | dichlorophenyl)propyl) | | (m, 2H), 1.10 (t, J = 7.2 | |
| | amino)isoquinolin- | | Hz, 3H). | |
| | 1(2H)-one | | | |
| Example 144 Method 2 Mixture of Stereoisomers | 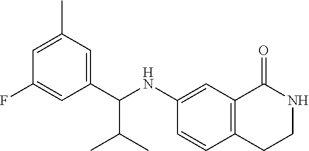 [SMILES: Cc1cc(F)cc(C(Nc2ccc3c(c2)C(=O)NCC3)C(C)C)c1] | White solid, yield: 4.00% | ¹H NMR (400 MHz, DMSO-d₆) δ 7.68 (d, J = 2.9 Hz, 1H), 7.09 (d, J = 2.5 Hz, 1H), 7.00 (s, 1H), 6.94 (d, J = 9.4 Hz, 1H), 6.89 (d, J = 8.2 Hz, 1H), 6.81 (d, J = 9.7 Hz, 1H), 6.65 (dd, J = 8.2, 2.6 Hz, 1H), 6.11 (d, J = 8.3 Hz, | 327.2 |
| | 7-((1-(3-fluoro-5- | | 1H), 4.07 (t, J = 7.7 Hz, | |
| | methylphenyl)-2- | | 1H), 3.29-3.20 (m, | |
| | methylpropyl)amino)- | | 2H), 2.64 (t, J = 6.5 Hz, | |
| | 3,4- | | 2H), 2.27 (s, 3H), 1.98- | |
| | dihydroisoquinolin- | | 1.89 (m, 1H), 0.97 (d, | |
| | 1(2H)-one | | J = 6.6 Hz, 3H), 0.79 | |
| | | | (d, J = 6.7 Hz, 3H). | |
| Example 145 Method 2 Mixture of Stereoisomers | 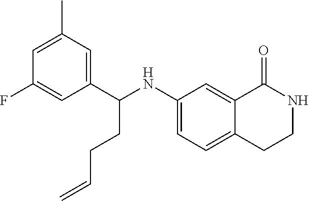 [SMILES: C=CCCC(Nc1ccc2c(c1)C(=O)NCC2)c1cc(C)cc(F)c1] | white solid, yield: 11.0% | 1H NMR (400 MHz, DMSO-d₆) δ 7.70 (d, J = 3.2 Hz, 1H), 7.06- 6.99 (m, 2H), 6.95 (d, J = 10.0, 1H), 6.91 (d, J = 8.0 Hz, 1H), 6.85-6.79 (m, 1H), 6.62 (dd, J = 8.2, 2.6 Hz, 1H), 6.25 (d, J = 8.0 Hz, 1H), 5.91-5.75 (m, 1H), 5.04-4.92 (m, 2H), 4.36-4.28 (m, 1H), 3.28-3.19 (m, 2H), | 339.2 |
| | 7-((1-(3-fluoro-5- | | 2.65 (t, J = 6.4 Hz, 2H), | |
| | methylphenyl)pent-4- | | 2.28 (s, 3H), 2.20- | |
| | en-1-yl)amino)-3,4- | | 2.00 (m, 2H), 1.87- | |
| | dihydroisoquinolin- | | 1.64 (m, 2H). | |
| | 1(2H)-one | | | |
| Example 146 Method 2 Mixture of Stereoisomers | 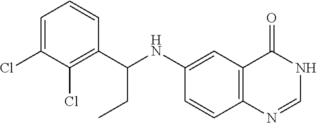 [SMILES: CCC(Nc1ccc2nc[nH]c(=O)c2c1)c1cccc(Cl)c1Cl] | White solid, yield: 6.7% | ¹H NMR (400 MHz, Methanol-d₄) δ 7.80 (s, 1H), 7.43 (d, J = 8.8 Hz, 1H), 7.41-7.34 (m, 2H), 7.20 (d, J = 8.0 Hz, 1H), 7.18-7.12 (m, 1H), 7.02 (d, J = 2.8 Hz, 1H), 4.84-4.80 | 348.3 |
| | 6-[1-(2,3- | | (m, 1H), 1.96-1.72 | |
| | dichlorophenyl) | | (m, 2H), 1.10 (t, J = 7.2 | |
| | propylamino]-3H- | | Hz, 3H). | |
| | quinazolin-4-one | | | |
| Example 147 Method 2 Mixture of Stereoisomers | 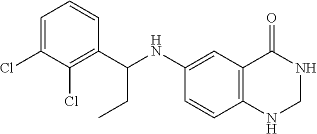 [SMILES: CCC(Nc1ccc2c(c1)C(=O)NCN2)c1cccc(Cl)c1Cl] | White solid, yield: 8.7% | ¹H NMR (400 MHz, Methanol-d₄) δ 7.40- 7.34 (m, 2H), 7.21- 7.15 (m, 1H), 6.93 (d, J = 2.4 Hz, 1H), 6.68- 6.56 (m, 2H), 4.73 (dd, J = 8.0, 5.2 Hz, 1H), 4.38 (s, 2H), 1.90- 1.69 (m, 2H), 1.05 (t, | 350.3 |
| | 6-((1-(2,3- | | J = 7.2 Hz, 3H). | |
| | dichlorophenyl)propyl) | | | |
| | amino)-2,3- | | | |
| | dihydroquinazolin- | | | |
| | 4(1H)-one | | | |
| Example 148 Method 2 Mixture of Stereoisomers | 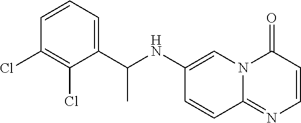 [SMILES: CC(Nc1ccc2nccc(=O)n2c1)c1cccc(Cl)c1Cl] | White solid, yield: 6.5% | ¹H NMR (400 MHz, DMSO-d₆) δ 8.09 (d, J = 6.0 Hz, 1H), 7.70- 7.61 (m, 2H), 7.61- 7.51 (m, 2H), 7.44 (dd, J = 8.0, 1.6 Hz, 1H), 7.33 (t, J = 8.0 Hz, 1H), 7.24 (d, J = 6.6 Hz, | 334.1 |
| | 7-((1-(2,3- | | 1H), 6.15 (d, J = 6.0 | |
| | dichlorophenyl)ethyl) | | Hz, 1H), 4.84 (p, J = | |
| | amino)-4H- | | 6.6 Hz, 1H), 1.50 (d, | |
| | pyrido[1,2- | | J = 6.6 Hz, 3H) | |
| | a]pyrimidin-4-one | | | |
| Example 149 Method 2 Mixture of Stereoisomers | 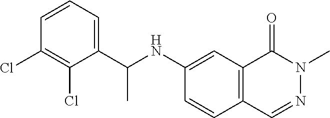 [SMILES: CC(Nc1ccc2cnn(C)c(=O)c2c1)c1cccc(Cl)c1Cl] | White solid, yield: 9.9% | ¹H NMR (400 MHz, DMSO-d₆) δ 8.06 (s, 1H), 7.61 (d, J = 8.6 Hz, 1H), 7.59-7.49 (m, 2H), 7.39 (dd, J = 8.0, 1.6 Hz, 1H), 7.31 (t, J = 7.8 Hz, 1H), 7.10 (dd, J = 8.8, 2.4 Hz, | 348.1 |
| | 7-((1-(2,3- | | 1H), 6.94 (s, 1H), 4.96 | |
| | dichlorophenyl)ethyl) | | (p, J = 6.6 Hz, 1H), | |
| | amino)-2- | | 3.60 (s, 3H), 1.49 (d, | |
| | methylphthalazin- | | J = 6.6 Hz, 3H). | |
| | 1(2H)-one | | | |
| Example 150 Method 2 Mixture of Stereoisomers | 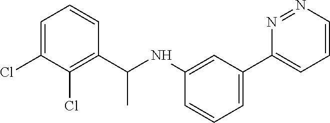 [SMILES: CC(Nc1cccc(-c2cccnn2)c1)c1cccc(Cl)c1Cl] | yellow solid, yield: 6.8% | ¹H NMR (400 MHz, DMSO-d₆) δ 9.16 (dd, J = 4.8, 1.6 Hz, 1H), 7.99 (dd, J = 8.8, 1.6 Hz, 1H), 7.73 (dd, J = 8.6, 4.8 Hz, 1H), 7.53-7.43 (m, 2H), 7.37 (s, 1H), 7.31 (t, J = 8.0 Hz, 1H), | 344.0 |
| | N-(1-(2,3- | | 7.23-7.16 (m, 2H), | |
| | dichlorophenyl)ethyl)- | | 6.74 (d, J = 7.2 Hz, | |
| | 3-(pyridazin-3- | | 1H), 6.47 (dt, J = 6.8, | |
| | yl)aniline | | 2.2 Hz, 1H), 4.93 (p, | |
| | | | J = 6.8 Hz, 1H), 1.47 | |
| | | | (d, J = 6.8 Hz, 3H). | |
| Example 151 Method 2 Mixture of Stereoisomers | 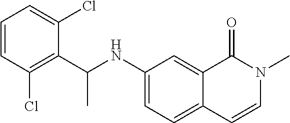 [SMILES: CC(Nc1ccc2ccn(C)c(=O)c2c1)c1c(Cl)cccc1Cl] | Light brown solid, yield: 5.8% | ¹H NMR (400 MHz, DMSO-d₆) δ 6.49 (d, J = 8.6 Hz, 3H), 6.40 (t, J = 8.0 Hz, 1H), 6.30 (d, J = 2.4 Hz, 1H), 6.25 (d, J = 7.2 Hz, 1H), 6.19 (dd, J = 8.6, 2.6 Hz, 1H), 5.88 (d, J = | 347.2 |
| | 7-((1-(2,6- | | 7.8 Hz, 1H), 5.52 (d, | |
| | dichlorophenyl)ethyl) | | J = 7.2 Hz, 1H), 4.52- | |
| | amino)-2- | | 4.41 (m, 1H), 2.57 (s, | |
| | methylisoquinolin- | | 3H), 0.78 (d, J = 7.0 | |
| | 1(2H)-one | | Hz, 3H). | |
| Example 152 Method 2 single unknown stereoisomer | 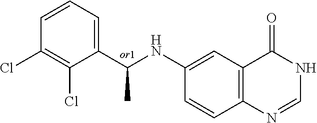 [SMILES: C[C@H](Nc1ccc2nc[nH]c(=O)c2c1)c1cccc(Cl)c1Cl] | White solid, yield: 9.2% | ¹H NMR (400 MHz, DMSO-d₆) δ 11.86 (s, 1H), 7.75 (s, 1H), 7.51 (dd, J = 7.8, 1.6 Hz, 1H), 7.44-7.38 (m, 2H), 7.30 (t, J = 7.8 Hz, 1H), 7.09 (dd, J = 8.8, 2.8 Hz, 1H), 7.02 (d, | 334.1 Note: Chiral separated from 128. SFC Thar prep 80 CHIRALPAK WHELK-01 |
| | rel-(R)-6-((1-(2,3- | | J = 7.0 Hz, 1H), 6.82 (d, | 250 mm * |
| | dichlorophenyl)ethyl) | | J = 2.8 Hz, 1H), 4.89 | 21 mm, 5 μm |
| | amino)quinazolin- | | (q, J = 6.7 Hz, 1H), | Modifier: |
| | 4(3H)-one | | 1.47 (d, J = 6.6 Hz, | 40% ETOH |
| | | | 3H). | (NH₄OH |
| | | | | 0.2%). Rt = |
| | | | | 3.64 min |
| Example 152A Method 2 single unknown stereoisomer | 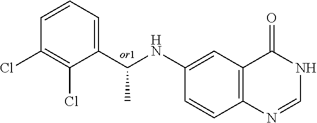 [SMILES: C[C@@H](Nc1ccc2nc[nH]c(=O)c2c1)c1cccc(Cl)c1Cl] | White solid, yield: 42.1% | ¹H NMR (400 MHz, DMSO-d₆) δ 11.86 (s, 1H), 7.75 (s, 1H), 7.51 (dd, J = 7.8, 1.7 Hz, 1H), 7.46-7.38 (m, 2H), 7.30 (t, J = 7.9 Hz, 1H), 7.09 (dd, J = 8.8, 2.8 Hz, 1H), 7.02 (d, | 334.1 Note: Chiral separated from 128. SFC Thar prep 80 CHIRALPAK WHELK-01 |
| | Rel-(S)-6-((1-(2,3- | | J = 7.0 Hz, 1H), 6.82 (d, | 250 mm * |
| | dichlorophenyl)ethyl) | | J = 2.8 Hz, 1H), 4.89 | 21 mm, 5 μm |
| | amino)quinazolin- | | (p, J = 6.7 Hz, 1H), | Modifier: |
| | 4(3H)-one | | 1.47 (d, J = 6.6 Hz, | 40% ETOH |
| | | | 3H). | (NH₄OH |
| | | | | 0.2%). Rt = |
| | | | | 4.61 min |
| Example 153 Method 2 Mixture of Stereoisomers | 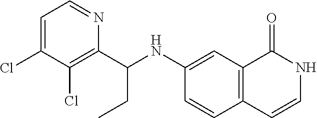 [SMILES: CCC(Nc1ccc2cc[nH]c(=O)c2c1)c1nccc(Cl)c1Cl] | yellow solid, yield: 8.6% | ¹H NMR (400 MHz, DMSO-d₆) δ 10.85 (d, J = 5.4 Hz, 1H), 8.45 (d, J = 5.1 Hz, 1H), 7.62 (d, J = 5.1 Hz, 1H), 7.33 (d, J = 8.6 Hz, 1H), 7.16 (d, J = 2.5 Hz, 1H), 7.10 (dd, J = | 348.3 |
| | 7-((1-(3,4- | | 8.6, 2.6 Hz, 1H), 6.80 | |
| | dichloropyridin-2- | | (dd, J = 7.1, 5.7 Hz, | |
| | yl)propyl)amino) | | 1H), 6.58 (d, J = 9.1 | |
| | isoquinolin-1(2H)-one | | Hz, 1H), 6.31 (d, J = | |
| | | | 6.9 Hz, 1H), 5.06-4.91 | |
| | | | (m, 1H), 1.94-1.81 | |
| | | | (m, 2H), 0.96 (t, J = 7.4 | |
| | | | Hz, 3H). | |
| Example 154 Method 2 Mixture of Stereoisomers | 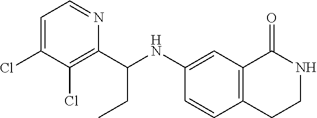 [SMILES: CCC(Nc1ccc2c(c1)C(=O)NCC2)c1nccc(Cl)c1Cl] | White solid, yield: 10.4% | ¹H NMR (400 MHz, Methanol-d₄) δ 8.36 (d, J = 5.1 Hz, 1H), 7.44 (d, J = 5.1 Hz, 1H), 7.21 (d, J = 2.6 Hz, 1H), 6.98 (d, J = 8.2 Hz, 1H), 6.78 (dd, J = 8.2, 2.6 Hz, 1H), 5.07 | 350.3 |
| | 7-((1-(3,4- | | (d, J = 6.8 Hz, 1H), | |
| | dichloropyridin-2- | | 3.39 (t, J = 6.7 Hz, 2H), | |
| | yl)propyl)amino)- | | 2.78 (t, J = 6.7 Hz, 2H), | |
| | 3,4- | | 1.98-1.82 (m, 2H), | |
| | dihydroisoquinolin- | | 1.00 (t, J = 7.4 Hz, 3H). | |
| | 1(2H)-one | | | |
| Example 155 Method 2 Mixture of Stereoisomers | 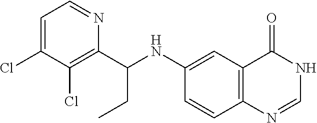 [SMILES: CCC(Nc1ccc2nc[nH]c(=O)c2c1)c1nccc(Cl)c1Cl] | White solid, yield: 9.6% | ¹H NMR (400 MHz, Methanol-d₄) δ 8.37 (d, J = 5.1 Hz, 1H), 7.81 (s, 1H), 7.51-7.39 (m, 2H), 7.25 (dd, J = 8.8, 2.8 Hz, 1H), 7.19 (d, J = 2.8 Hz, 1H), 5.12 (t, J = 6.7 Hz, 1H), 2.01- | 349.2 |
| | 6-((1-(3,4-dichloropyridin-2- | | 1.90 (m, 2H), 1.04 (t, | |
| | yl)propyl)amino) | | J = 7.4 Hz, 3H). | |
| | quinazolin-4(3H)-one | | | |
| Example 156 Method 2 Mixture of Stereoisomers | 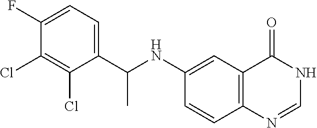 [SMILES: CC(Nc1ccc2nc[nH]c(=O)c2c1)c1ccc(F)c(Cl)c1Cl] | White solid, yield: 11.2% | ¹H NMR (400 MHz, Methanol-d₄) δ 7.80 (s, 1H), 7.49-7.39 (m, 2H), 7.20-7.10 (m, 2H), 6.95 (d, J = 2.8 Hz, 1H), 4.96 (q, J = 6.7 Hz, 1H), 1.54 (d, J = 6.6 Hz, 3H). | 352.2 |
| | 6-[1-(2,3-dichloro-4- | | | |
| | fluoro- | | | |
| | phenyl)ethylamino]- | | | |
| | 3H-quinazolin-4-one | | | |
| Example 157 Method 2 Mixture of Stereoisomers | 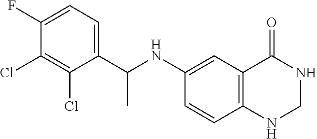 [SMILES: CC(Nc1ccc2c(c1)C(=O)NCN2)c1ccc(F)c(Cl)c1Cl] | yellow solid, yield: 12.7% | ¹H NMR (400 MHz, Methanol-d₄) δ 7.44 (dd, J = 8.8, 5.8 Hz, 1H), 7.12 (t, J = 8.6 Hz, 1H), 6.87 (s, 1H), 6.61 (s, 2H), 4.91-4.84 (m, 1H), 4.39 (s, 2H), 1.46 (d, J = 6.6 Hz, 3H). | 354.3 |
| | 6-((1-(2,3-dichloro- | | | |
| | 4- | | | |
| | fluorophenyl)ethyl) | | | |
| | amino)-2,3- | | | |
| | dihydroquinazolin- | | | |
| | 4(1H)-one | | | |
| Example 192 Method 2 Mixture of Stereoisomers | 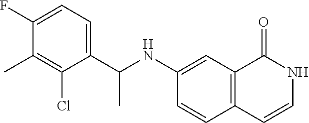 [SMILES: Cc1c(F)ccc(C(C)Nc2ccc3cc[nH]c(=O)c3c2)c1Cl] | White solid, yield: 26.01% | ¹H NMR (400 MHz, DMSO-d6) δ 10.82 (d, J = 5.6 Hz, 1H), 7.38- 7.29 (m, 2H), 7.11 (t, J = 8.9 Hz, 1H), 7.03- 6.95 (m, 2H), 6.83- 6.72 (m, 2H), 6.31 (d, J = 7.0 Hz, 1H), 4.91- | 331.02 |
| | 7-[1-(2-chloro-4- | | 4.80 (m, 1H), 2.31 (d, | |
| | fluoro-3-methyl- | | J = 2.3 Hz, 3H), 1.44 (d, | |
| | phenyl)ethylamino]- | | J = 6.6 Hz, 3H). | |
| | 2H-isoquinolin-1-one | | | |
| Example 193 Method 2 Mixture of Stereoisomers | 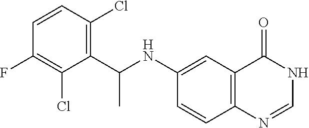 [SMILES: CC(Nc1ccc2nc[nH]c(=O)c2c1)c1c(Cl)ccc(F)c1Cl] | white solid, yield: 23.0% | ¹H NMR (400 MHz, DMSO-d₆) δ 11.82 (s, 1H), 7.74 (s, 1H), 7.59- 7.42 (m, 1H), 7.41- 7.32 (m, 2H), 7.12 (dd, J = 8.9, 2.7 Hz, 1H), 6.91 (d, J = 7.0 Hz, 2H), 5.28 (t, J = 7.1 Hz, | 352.0 |
| | 6-((1-(2,6-dichloro- | | 1H), 1.62 (d, J = 7.0 | |
| | 3- | | Hz, 3H). | |
| | fluorophenyl)ethyl) | | | |
| | amino)quinazolin- | | | |
| | 4(3H)-one | | | |
| Example 194 Method 2 Mixture of Stereoisomers | 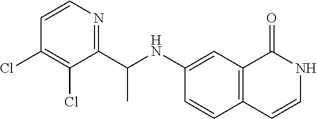 [SMILES: CC(Nc1ccc2cc[nH]c(=O)c2c1)c1nccc(Cl)c1Cl] | White solid, yield: 15.2% | ¹H NMR (400 MHz, DMSO-d₆) δ 10.86 (d, J = 5.6 Hz, 1H), 8.45 (d, J = 5.2 Hz, 1H), 7.63 (d, J = 5.1 Hz, 1H), 7.34 (d, J = 8.5 Hz, 1H), 7.12 (d, J = 2.5 Hz, 1H), 7.08 (dd, J = | 334.0 |
| | 7-((1-(3,4- | | 8.5, 2.6 Hz, 1H), 6.81 | |
| | dichloropyridin-2- | | (dd, J = 7.0, 5.7 Hz, | |
| | yl)ethyl)amino) | | 1H), 6.63 (d, J = 8.8 | |
| | isoquinolin-1(2H)-one | | Hz, 1H), 6.32 (d, J = | |
| | | | 7.0 Hz, 1H), 5.22-5.12 | |
| | | | (m, 1H), 1.48 (d, J = | |
| | | | 6.6 Hz, 3H). | |
| Example 194A Method 2 single unknown stereoisomer | 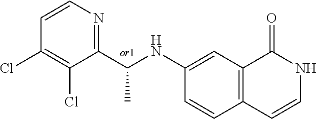 [SMILES: C[C@@H](Nc1ccc2cc[nH]c(=O)c2c1)c1nccc(Cl)c1Cl] | White solid, yield: 15.2% | ¹H NMR (400 MHz, DMSO-d₆) δ 10.86 (d, J = 5.6 Hz, 1H), 8.45 (d, J = 5.2 Hz, 1H), 7.63 (d, J = 5.1 Hz, 1H), 7.34 (d, J = 8.5 Hz, 1H), 7.12 (d, J = 2.5 Hz, 1H), 7.08 (dd, J = | 334.0 Note: Chiral separated from 118, CHIRALPAK IC, 2 cm * 25 cm, 5 um, |
| | (S or R)-7-((1-(3,4- | | 8.5, 2.6 Hz, 1H), 6.81 | Hex:DCM, |
| | dichloropyridin-2- | | (dd, J = 7.0, 5.7 Hz, | EtOH, |
| | yl)ethyl)amino) | | 1H), 6.63 (d, J = 8.8 | 20 mL/min, |
| | isoquinolin-1(2H)-one | | Hz, 1H), 6.32 (d, J = | Rt = 3.091 |
| | | | 7.0 Hz, 1H), 5.22-5.12 | min |
| | | | (m, 1H), 1.48 (d, J = | |
| | | | 6.6 Hz, 3H). | |
| Example 194B Method 2 single unknown stereoisomer | 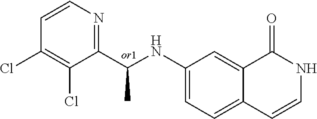 [SMILES: C[C@H](Nc1ccc2cc[nH]c(=O)c2c1)c1nccc(Cl)c1Cl] | White solid, | ¹H NMR (400 MHz, DMSO-d₆) δ 10.86 (d, J = 5.6 Hz, 1H), 8.45 (d, J = 5.2 Hz, 1H), 7.63 (d, J = 5.1 Hz, 1H), 7.34 (d, J = 8.5 Hz, 1H), 7.12 (d, J = 2.5 Hz, 1H), 7.08 (dd, J = | 334.0 Note: Chiral separated from 118, CHIRALPAK IC, 2 cm * 25 cm, 5 um, |
| | Rel-(R)-7-((1-(3,4- | | 8.5, 2.6 Hz, 1H), 6.81 | Hex:DCM, |
| | dichloropyridin-2- | | (dd, J = 7.0, 5.7 Hz, | EtOH, |
| | yl)ethyl)amino) | | 1H), 6.63 (d, J = 8.8 | 20 mL/min, |
| | isoquinolin-1(2H)-one | | Hz, 1H), 6.32 (d, J = | Rt = 4.151 |
| | | | 7.0 Hz, 1H), 5.22-5.12 | min |
| | | | (m, 1H), 1.48 (d, J = | |
| | | | 6.6 Hz, 3H). | |
| Example 195 Method 2 Mixture of Stereoisomers | 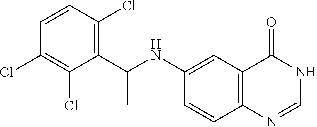 [SMILES: CC(Nc1ccc2nc[nH]c(=O)c2c1)c1c(Cl)ccc(Cl)c1Cl] | White solid, yield: 10.1% | ¹H NMR (400 MHz, DMSO-d₆) δ 11.81 (s, 1H), 7.73 (s, 1H), 7.56 (d, J = 8.2 Hz, 1H), 7.38 (d, J = 8.8 Hz, 1H), 7.11 (dd, J = 8.8, 2.8 Hz, 1H), 6.97-6.88 (m, 2H), 5.32 (q, J = | 368.1 |
| | 6-((1-(2,3,6- | | 7.2 Hz, 1H), 1.61 (d, | |
| | trichlorophenyl)ethyl) | | J = 7.0 Hz, 3H). | |
| | amino)quinazolin- | | | |
| | 4(3H)-one | | | |
| Example 198 Method 2 Mixture of Stereoisomers | 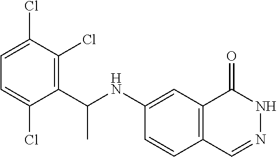 [SMILES: CC(Nc1ccc2cn[nH]c(=O)c2c1)c1c(Cl)ccc(Cl)c1Cl] | White solid, yield: 9.5% | ¹H NMR (400 MHz, DMSO-d₆) δ 11.81 (s, 1H), 7.73 (s, 1H), 7.56 (d, J = 8.2 Hz, 1H), 7.38 (d, J = 8.8 Hz, 1H), 7.11 (dd, J = 8.8, 2.8 Hz, 1H), 6.97-6.88 (m, 2H), 5.32 (q, J = 7.2 Hz, 1H), 1.61 (d, J = 7.0 Hz, 3H). | 368.1 |
| | 7-((1-(2,3,6- | | | |
| | trichlorophenyl)ethyl) | | | |
| | amino)phthalazin- | | | |
| | 1(2H)-one | | | |
| Example 199 Method 2 Mixture of Stereoisomers | 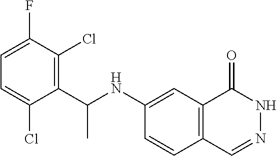 [SMILES: CC(Nc1ccc2cn[nH]c(=O)c2c1)c1c(Cl)ccc(F)c1Cl] | White solid, yield: 9.1% | ¹H NMR (400 MHz, DMSO-d₆) δ 11.81 (s, 1H), 7.73 (s, 1H), 7.56 (d, J = 8.2 Hz, 1H), 7.38 (d, J = 8.8 Hz, 1H), 7.11 (dd, J = 8.8, 2.8 Hz, 1H), 6.97-6.88 (m, 2H), 5.32 (q, J = 7.2 Hz, 1H), 1.61 (d, J = 7.0 Hz, 3H) | 352.1 |
| | 7-((1-(2,3,6- | | | |
| | trichlorophenyl)ethyl) | | | |
| | amino)phthalazin- | | | |
| | 1(2H)-one | | | |
| Example 200 Method 2 Mixture of Stereoisomers | 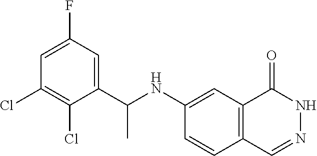 [SMILES: CC(Nc1ccc2cn[nH]c(=O)c2c1)c1cc(F)cc(Cl)c1Cl] | White solid, yield: 10.3% | ¹H NMR (400 MHz, DMSO-d₆) δ 12.25 (s, 1H), 8.04 (s, 1H), 7.66- 7.58 (m, 2H), 7.48 (d, J = 7.0 Hz, 1H), 7.26 (dd, J = 9.3, 3.0 Hz, 1H), 7.11 (dd, J = 8.7, 2.5 Hz, 1H), 6.92 (s, 1H), 4.96 (q, J = 7.2 Hz, 1H), 1.49 (d, J = | 352.1 |
| | 7-((1-(2,3-dichloro- | | 6.6 Hz, 3H). | |
| | 5- | | | |
| | fluorophenyl)ethyl) | | | |
| | amino)phthalazin- | | | |
| | 1(2H)-one | | | |
| Example 201 Method 2 single unknown stereoisomer | 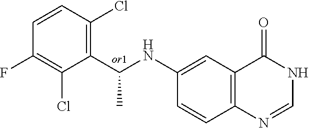 [SMILES: C[C@@H](Nc1ccc2nc[nH]c(=O)c2c1)c1c(Cl)ccc(F)c1Cl] | white solid, yield: 12.0% | ¹H NMR (400 MHz, DMSO-d₆) δ 11.82 (s, 1H), 7.73 (s, 1H), 7.47 (s, 1H), 7.41-7.32 (m, 2H), 7.11 (dd, J = 8.8, 2.8 Hz, 1H), 6.97-6.86 (m, 2H), 5.27 (p, J = 7.1 Hz, 1H), 1.62 (d, | 352.0 Note: CHIRAL Cellulose SB, 2 cm * 25 cm, 5 um, MeOH:DCM, |
| | rel-(R)-6-((1-(2,6- | | J = 7.0 Hz, 3H). | MtBE, |
| | dichloro-3- | | | 20 mL/min, |
| | fluorophenyl)ethyl) | | | Rt = 3.522 |
| | amino)quinazolin- | | | min |
| | 4(3H)-one | | | |
| Example 201A Method 2 single unknown stereoisomer | 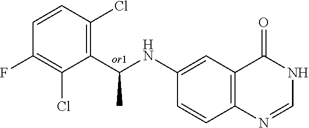 [SMILES: C[C@H](Nc1ccc2nc[nH]c(=O)c2c1)c1c(Cl)ccc(F)c1Cl] | white solid, yield: 12% | ¹H NMR (400 MHz, DMSO-d₆) δ 11.82 (s, 1H), 7.74 (s, 1H), 7.59-7.42 (m, 1H), 7.41-7.32 (m, 2H), 7.12 (dd, J = 8.9, 2.7 Hz, 1H), 6.91 (d, J = 7.0 Hz, 2H), 5.28 (t, J = 7.1 Hz, 1H), 1.62 (d, J = 7.0 Hz, 3H). | 352.0 Note: CHIRAL Cellulose SB, 2 cm * 25 cm, 5 um, MeOH:DCM, |
| | rel-(S)-6-((1-(2,6- | | | MtBE, |
| | dichloro-3- | | | 20 mL/min, |
| | fluorophenyl)ethyl) | | | Rt = 1.69 |
| | amino)quinazolin- | | | min |
| | 4(3H)-one | | | |
| Example 203 Method 2 Mixture of Stereoisomers | 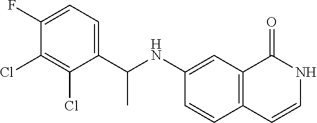 [SMILES: CC(Nc1ccc2cc[nH]c(=O)c2c1)c1ccc(F)c(Cl)c1Cl] | white solid, yield: 24.5% | ¹H NMR (400 MHz, Methanol-d₄) δ 7.49- 7.36 (m, 2H), 7.19- 7.02 (m, 3H), 6.87 (d, J = 7.1 Hz, 1H), 6.52 (d, J = 7.1 Hz, 1H), 4.97 (q, J = 6.7 Hz, 1H), 1.53 (d, J = 6.6 Hz, 3H). | 351.2 |
| | 7-[1-(2,3-dichloro-4- | | | |
| | fluoro- | | | |
| | phenyl)ethylamino]- | | | |
| | 2H-isoquinolin-1-one | | | |
| Example 205 Method 2 Mixture of Stereoisomers | 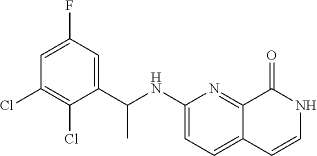 [SMILES: CC(Nc1ccc2cc[nH]c(=O)c2n1)c1cc(F)cc(Cl)c1Cl] | White solid, yield: 18.5% | ¹H NMR (400 MHz, DMSO-d₆) δ 11.13 (brs, 1H), 7.75-7.64 (m, 2H), 7.56 (dd, J = 8.0, 3.2 Hz, 1H), 7.34 (dd, J = 9.6, 3.2 Hz, 1H), 6.90 (t, J = 6.5 Hz, 2H), 6.30 (d, J = 6.9 Hz, 1H), 5.59-5.51 (m, 1H), 1.45 (d, J = 6.8 Hz, 3H). | 352.4 |
| | 2-((1-(2,3-dichloro- | | | |
| | 5- | | | |
| | fluorophenyl)ethyl) | | | |
| | amino)-1,7- | | | |
| | naphthyridin-8(7H)- | | | |
| | one | | | |
| Example 210 Method 2 Mixture of Stereoisomers | 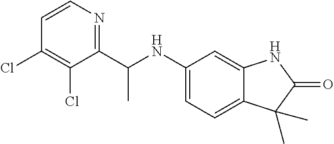 [SMILES: CC(Nc1ccc2c(c1)NC(=O)C2(C)C)c1nccc(Cl)c1Cl] | white solid, yield: 15.04% | ¹H NMR (400 MHz, DMSO-d₆) δ 10.01 (s, 1H), 8.46 (d, J = 5.2 Hz, 1H), 7.63 (d, J = 5.2 Hz, 1H), 6.86 (d, J = 8.0 Hz, 1H), 6.18 (d, J = 8.8 Hz, 1H), 6.12 (d, J = 2.0 Hz, 1H), 6.07 (dd, J = 8.0, 2.0 | 350.0/352.0 |
| | 6-((1-(3,4- | | Hz, 1H), −5.06-4.97 | |
| | dichloropyridin-2- | | (m, 1H), 1.42 (d, J = | |
| | yl)ethyl)amino)-3,3- | | 6.8 Hz, 3H), 1.12 (d, | |
| | dimethylindolin-2- | | J = 3.2 Hz, 6H). | |
| | one | | | |
| Example 213 Method 2 Mixture of Stereoisomers | 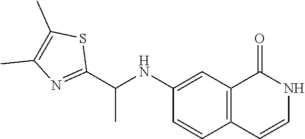 [SMILES: Cc1nc(C(C)Nc2ccc3cc[nH]c(=O)c3c2)sc1C] | white solid, yield: 15.1% | ¹H NMR (400 MHz, DMSO-d₆) δ 10.91 (d, J = 5.5 Hz, 1H), 7.38 (d, J = 8.6 Hz, 1H), 7.16 (d, J = 2.5 Hz, 1H), 7.08 (dd, J = 8.6, 2.6 Hz, 1H), 6.84 (dd, J = 7.0, 5.6 Hz, 1H), 6.75 (d, J = 6.3 Hz, 1H), | 300.2 |
| | 7-((1-(4,5- | | 6.35 (d, J = 7.0 Hz, | |
| | dimethylthiazol-2- | | 1H), 4.72 (p, J = 6.6 | |
| | yl)ethyl)amino) | | Hz, 1H), 2.25-2.20 (m, | |
| | isoquinolin-1(2H)-one | | 6H), 1.53 (d, J = 6.7 | |
| | | | Hz, 3H). | |
| Example 221 Method 2 Mixture of Stereoisomers | 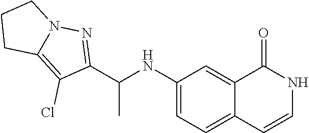 [SMILES: CC(Nc1ccc2cc[nH]c(=O)c2c1)c1nn2c(c1Cl)CCC2] | white solid, yield: 3.7% | ¹H NMR (400 MHz, DMSO-d₆) δ 10.86 (d, J = 5.6 Hz, 1H), 8.15 (s, 1H), 7.33 (d, J = 8.8 Hz, 1H), 7.24 (d, J = 2.4 Hz, 1H), 7.08 (dd, J = 8.8, 2.4 Hz, 1H), 6.81 (dd, J = 7.2, 5.6 Hz, 1H), 6.33 (dd, J = 7.6, | 329.0/331.0 |
| | 7-((1-(3-chloro-5,6- | | 2.8 Hz, 2H), 4.62 (q, | |
| | dihydro-4H- | | J = 7.2 Hz, 1H), 4.08- | |
| | pyrrolo[1,2- | | 3.96 (m, 2H), 2.79 (dd, | |
| | b]pyrazol-2- | | J = 8.0, 6.4 Hz, 2H), | |
| | yl)ethyl)amino) | | 2.49-2.44 (m, 2H), | |
| | isoquinolin-1(2H)-one | | 1.49 (d, J = 6.8 Hz, | |
| | | | 3H). | |
| Example 222 Method 2 Mixture of Stereoisomers | 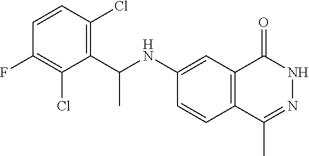 [SMILES: Cc1n[nH]c(=O)c2cc(NC(C)c3c(Cl)ccc(F)c3Cl)ccc12] | Yellow solid, yield: 34.1% | ¹H NMR (400 MHz, DMSO-d₆) δ 11.93 (br s, 1H), 7.60 (dd, J = 9.1, 1.3 Hz, 1H), 7.47 (br s, 1H), 7.44-7.32 (m, 2H), 7.07 (d, J = 5.9 Hz, 2H), 5.32 (t, J = 6.9 Hz, 1H), 2.34 (s, 3H), 1.63 (d, J = 7.0, 3H). | 366.2 |
| | 7-((1-(2,6-dichloro- | | | |
| | 3- | | | |
| | fluorophenyl)ethyl) | | | |
| | amino)-4- | | | |
| | methylphthalazin- | | | |
| | 1(2H)-one | | | |
| Example 223 Method 2 Mixture of Stereoisomers | 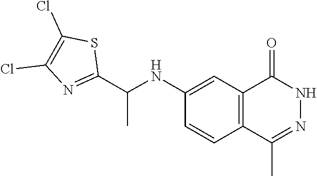 [SMILES: Cc1n[nH]c(=O)c2cc(NC(C)c3nc(Cl)c(Cl)s3)ccc12] | Yellow solid, yield: 31.4% | ¹H NMR (400 MHz, DMSO-d₆) δ 12.09 (br s, 1H), 7.70 (d, J = 8.6 Hz, 1H), 7.52 (d, J = 6.3 Hz, 1H), 7.28-7.17 (m, 2H), 5.00 (t, J = 6.6 Hz, 1H), 2.40 (s, 3H), 1.59 (d, J = 6.7 Hz, 3H). | 355.1 |
| | 7-((1-(4,5- | | | |
| | dichlorothiazol-2- | | | |
| | yl)ethyl)amino)-4- | | | |
| | methylphthalazin- | | | |
| | 1(2H)-one | | | |
| Example 237 Method 2 single unknown stereoisomer | 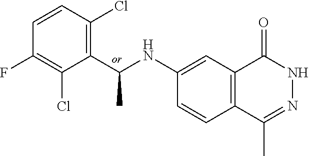 [SMILES: Cc1n[nH]c(=O)c2cc(N[C@@H](C)c3c(Cl)ccc(F)c3Cl)ccc12] | Yellow solid, yield: 35.2% | 1H NMR (400 MHz, Methanol-d₄) δ 7.64 (d, J = 8.8 Hz, 1H), 7.42- 7.31 (m, 1H), 7.23 (d, J = 2.6 Hz, 1H), 7.18- 7.08 (m, 2H), 5.55- 5.40 (m, 1H), 2.44 (s, 3H), 1.71 (d, J = 7.0 Hz, 3H). | 366.2 Note: SFC CHIRALPAK AD-3 250 mm × 20 mm, 5 μm Modifier: Hex(0.1% DEA): EtOH = |
| | 4-methyl-7-[[(1S)-1- | | | 70:30 |
| | (2,6-dichloro-3- | | | Rt = 4.2 min |
| | fluoro- | | | |
| | phenyl)ethyl]amino]- | | | |
| | 2H-phthalazin-1-one | | | |
| Example 237A Method 2 single unknown stereoisomer | 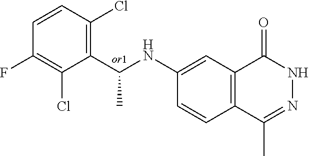 [SMILES: Cc1n[nH]c(=O)c2cc(N[C@H](C)c3c(Cl)ccc(F)c3Cl)ccc12] | white solid, yield: 24.3% | ¹H NMR (400 MHz, Methanol-d₄) δ 7.64 (d, J = 8.8 Hz, 1H), 7.35 (s, 1H), 7.23 (d, J = 2.6 Hz, 1H), 7.18-7.08 (m, 2H), 5.48 (q, J = 7.0 Hz, 1H), 2.44 (s, 3H), 1.71 (d, J = 7.0 Hz, 3H). | 366.2 Note: SFC CHIRALPAK AD-3 250 mm × 20 mm, 5 μm Modifier: Hex(0.1% DEA): EtOH = |
| | (R)-7-((1-(2,6- | | | 70:30 |
| | dichloro-3- | | | Rt = 5.05 min |
| | fluorophenyl)ethyl) | | | |
| | amino)-4- | | | |
| | methylphthalazin- | | | |
| | 1(2H)-one | | | |
| Example 239A Method 2 single unknown stereoisomer | 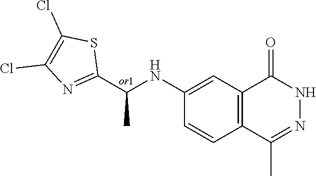 [SMILES: Cc1n[nH]c(=O)c2cc(N[C@@H](C)c3nc(Cl)c(Cl)s3)ccc12] | Yellow solid, yield: 32.5% | ¹H NMR (400 MHz, DMSO-d₆) δ 12.08 (br s, 1H), 7.70 (d, J = 8.6 Hz, 1H), 7.51 (d, J = 6.3 Hz, 1H), 7.28-7.16 (m, 2H), 5.00 (t, J = 6.6 Hz, 1H), 2.39 (s, 3H), 1.59 (d, J = 6.7 Hz, 3H). | 355.3 Note: SFC CHIRALPAK IF 250 mm × 20 mm, 5 μm Modifier: MtBE (0.1% DEA): EtOH = 90:10 Rt = 2.1 min |
| | (S)-7-((1-(4,5- | | | |
| | dichlorothiazol-2- | | | |
| | yl)ethyl)amino)-4- | | | |
| | methylphthalazin- | | | |
| | 1(2H)-one | | | |
| Example 239B Method 2 single unknown stereoisomer | 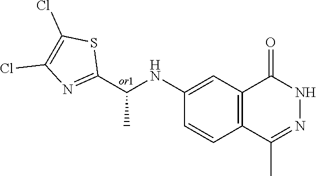 [SMILES: Cc1n[nH]c(=O)c2cc(N[C@H](C)c3nc(Cl)c(Cl)s3)ccc12] | White solid, yield: 33.8% | ¹H NMR (400 MHz, DMSO-d₆) δ 12.09 (s, 1H), 7.70 (d, J = 8.6 Hz, 1H), 7.52 (d, J = 6.3 Hz, 1H), 7.29-7.15 (m, 2H), 5.05-4.95 (m, 1H), 2.40 (s, 3H), 1.59 (d, J = 6.7 Hz, 3H). | 355.3 Note: SFC CHIRALPAK IF 250 mm × 20 mm, 5 μm Modifier : MtBE (0.1% DEA): EtOH = 90:10 Rt = 1.45 min |
| | rel-(R)-7-((1-(4,5- | | | |
| | dichlorothiazol-2- | | | |
| | yl)ethyl)amino)-4- | | | |
| | methylphthalazin- | | | |
| | 1(2H)-one | | | |
| Example 244 Method 2 Mixture of Stereoisomers | 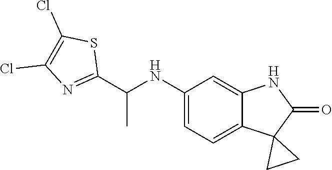 [SMILES: CC(Nc1ccc2c(c1)NC(=O)C21CC1)c1nc(Cl)c(Cl)s1] | yellow solid, yield: 21.8% | ¹H NMR (400 MHz, DMSO-d₆) δ 10.28 (s, 1H), 6.65 (d, J = 8.0 Hz, 1H), 6.50 (d, J = 5.6 Hz, 1H), 6.24 (d, J = 2.0 Hz, 1H), 6.15 (dd, J = 8.0, 2.0 Hz, 1H), 4.79-4.58 (m, 1H), 1.52 (d, J = 6.8 Hz, 3H), 1.39-1.27 (m, 4H). | 354.1/356.1 |
| | 6′-((1-(4,5- | | | |
| | dichlorothiazol-2- | | | |
| | yl)ethyl)amino)spiro | | | |
| | [cyclopropane-1,3′- | | | |
| | indolin]-2′-one | | | |
| Example 254 Method 2 Mixture of Stereoisomers | 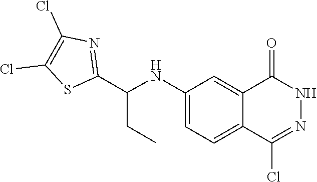 [SMILES: CCC(Nc1ccc2c(Cl)n[nH]c(=O)c2c1)c1nc(Cl)c(Cl)s1] | White solid, yield: 8.9% | ¹H NMR (400 MHz, Methanol-d₄) δ 7.86 (d, J = 8.6 Hz, 1H), 7.37- 7.31 (m, 2H), 4.75 (m, 1H), 2.18-2.03 (m, 2H), 1.11 (t, J = 7.4 Hz, 3H). | 389.31 |
| | 4-chloro-7-[1-(4,5- | | | |
| | dichlorothiazol-2- | | | |
| | yl)propylamino]-2H- | | | |
| | phthalazin-1-one | | | |

### Preparation of N-(3-fluoro-2-methylphenyl)-1-oxo-1,2,3,4-tetrahydroisoquinoline-7-sulfinamide (158)

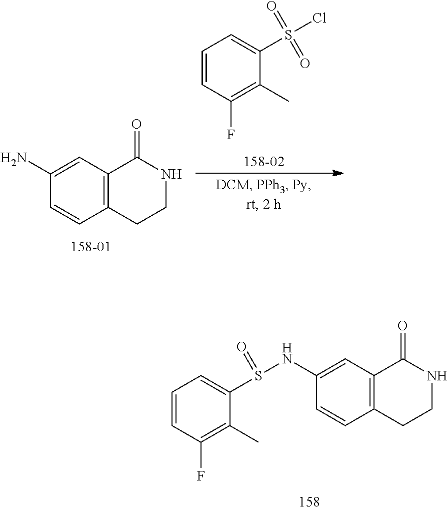 [SMILES: Nc1ccc2c(c1)C(=O)NCC2 ; Cc1c(F)cccc1S(=O)(=O)Cl ; Cc1c(F)cccc1S(=O)Nc1ccc2c(c1)C(=O)NCC2]

To a solution of 158-01 (150 mg, 0.604 mmol) in DCM (10 mL) was added 158-02 (126 mg, 0.604 mmol), Pyridine (143 mg, 1.81 mmol) and Triphenylphosphine (242 mg, 0.925 mmol). The mixture was stirred at 25° C. for 2 hours. The mixture was concentrated in vacuo. Purification by prep-HPLC gave the compound 158 (30 mg, 9.68%) as a white solid. Mass (nm/z): 318.3 [M+H]⁺. ¹H NMR (400 MHz, DMSO-d₆) δ 9.27 (s, 1H), 7.92 (s, 1H), 7.77 (d, J=8.4 Hz, 1H), 7.61 (d, J=2.4 Hz, 1H), 7.55 (d, J=5.6, 8.0 Hz, 1H), 7.42 (dd, J=1.2, 9.6 Hz, 1H), 7.21 (d, J=8.0 Hz, 1H), 7.18 (dd, J=2.4, 8.0 Hz, 1H) 3.35 (dd, J=6.4, 2.8 Hz, 2H), 2.81 (t, J=6.4 Hz, 2H), 2.24 (d, J=1.6 Hz, 3H).

### Preparation of 3-fluoro-2-methyl-N-(3-(thiazol-4-yl)phenyl)benzenesulfonamide (159) Method 3

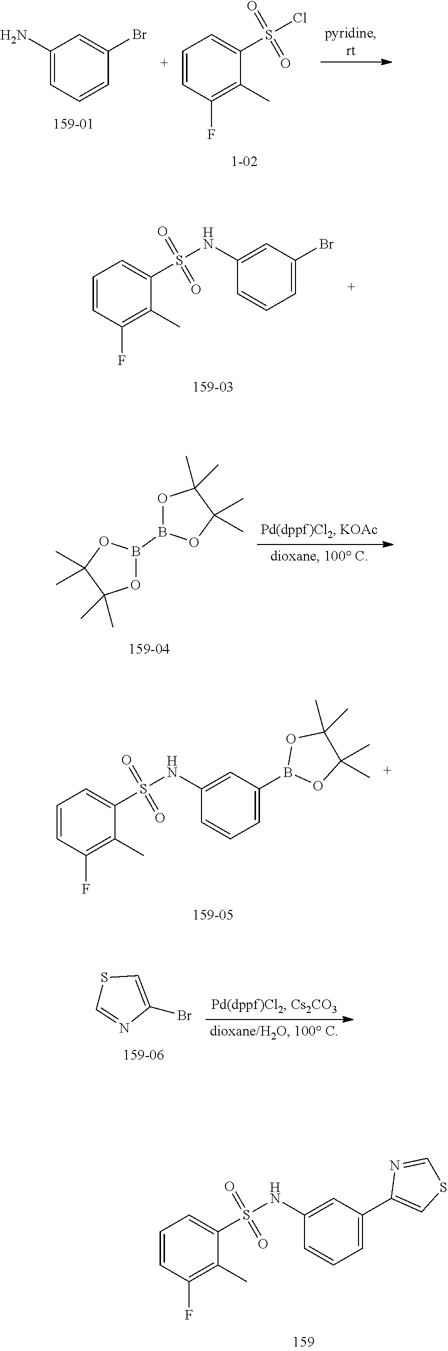

Step 1: To a solution of 3-bromoaniline 159-01 (5.00 g, 29.4 mmol) in pyridine (100 mL) was added 3-fluoro-2-methylbenzene-1-sulfonyl chloride 1-02 (7.30 g, 35.2 mmol) at room temperature. The mixture was stirred for 3 h at rt. The mixture was diluted with EtOAc, and washed with water. The organic layer was separated, dried over (Na₂SO₄), and concentrated under vacuum to give the desired product 159-03. MS (m/z): 344.0 [M+H]⁺.

Step 2: To a solution of 159-03 (5.00 g, 14.5 mmol) in dioxane (100 mL) were added ′4,′,4′,″,5,5′-hexamethyl-2,2′-bi(1,3,2-dioxaborolane)—159-04 (7.40 g, 29.1 mmol), Pd(dppf)Cl₂ (1.10 g, 1.5 mmol) and KOAc (4.30 g, 43.5 mmol) at rt under N₂. The mixture was stirred for 3 h at 100° C. under N₂. The mixture was diluted with EtOAc and washed with water. The organic layer was separated, dried over (Na₂SO₄), and concentrated under vacuum. Purification by silica gel chromatography gave the desired product 159-05. MS (m/z): 392.1 [M+H]⁺.

Step 3: To a suspension of 159-05 (619 mg, 14.5 mmol) in dioxane (20 mL) and H₂O (5 mL) was added 4-bromothiazole 159-06 (200 mg, 1.21 mmol), Cs₂CO₃ (786 mg, 2.21 mmol) and Pd(dppf)Cl₂ (88 mg, 0.12 mmol) at rt under N₂. The mixture was stirred for 2 h at 100° C. under N₂. The mixture was diluted with EtOAc, and washed with water. The organic layer was separated, dried (Na₂SO₄), and concentrated under vacuum. Purification by prep-HPLC gave the titled compound 159 (164.3 mg, 38.7%) as a white solid. MS (m/z): 348.9 [M+H]⁺. ¹H NMR (400 MHz, Methanol-d₄) δ 9.01 (d, J=2.0 Hz, 1H), 7.81-7.76 (m, 2H), 7.66 (d, J=2.0 Hz, 1H), 7.58 (d, J=8.0 Hz, 1H), 7.28-7.24 (m, 3H), 7.07 (d, J=6.4 Hz, 1H), 2.54 (d, J=2.0 Hz, 3H).

Compounds 160-162 were synthesized using a method like that used in Example 159.

| Example | | | | |
|---|---|---|---|---|
| (Compound) | | Appearance | | |
| No.) | | and | | MS (m/z) |
| Method | Structure and Name | Yield | ¹H NMR Data | [M + H]⁺ |
| Example 160 Method 3 | 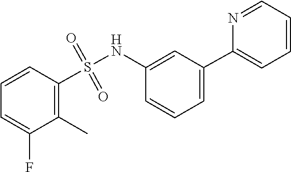 [SMILES: Cc1c(F)cccc1S(=O)(=O)Nc1cccc(-c2ccccn2)c1] | white solid, yield: 27.0% | ¹H NMR (400 MHz, DMSO-d₆) δ 8.65 (d, J = 4.4 Hz, 1H), 7.87- 7.76 (m, 4H), 7.66 (d, J = 8.0 Hz, 1H), 7.43- 7.30 (m, 4H), 7.13 (d, J = 7.6 Hz, 1H), 2.50 (s, 3H). | 343.0 |
| | 3-fluoro-2-methyl-N- | | | |
| | (3-(pyridin-2- | | | |
| | yl)phenyl)benzene- | | | |
| | sulfonamide | | | |
| Example 161 Method 3 | 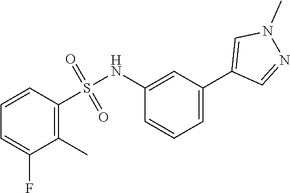 [SMILES: Cc1c(F)cccc1S(=O)(=O)Nc1cccc(-c2cnn(C)c2)c1] | white solid, yield: 61.2% | ¹H NMR (400 MHz, DMSO-d₆) δ 7.97 (s, 1H), 7.80-7.78 (m, 1H), 7.65 (s, 1H), 7.40-7.37 (m, 2H), 7.15-7.09 (m, 3H), 6.82 (dd, J = 5.6, 1.6 Hz, 1H), 3.84 (s, 3H), 2.48 (d, J = 2.4 Hz, 3H). | 346.0 |
| | 3-fluoro-2-methyl-N- | | | |
| | (3-(1-methyl-1H- | | | |
| | pyrazol-4- | | | |
| | yl)phenyl)benzene- | | | |
| | sulfonamide | | | |
| Example 162 Method 3 | 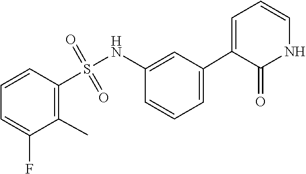 [SMILES: Cc1c(F)cccc1S(=O)(=O)Nc1cccc(-c2ccc[nH]c2=O)c1] | white solid, yield: 4.9% | ¹H NMR (400 MHz, DMSO-d₆) δ 7.72 (d, J = 7.6 Hz, 1H), 7.39 (dd, J = 2.0, 6.4 Hz, 1H), 7.33-7.21 (m, 4H), 7.07 (d, J = 8.0 Hz, 1H), 7.01 (t, J = 8.0 Hz, 1H), 6.84 (d, J = 7.6 Hz, 1H), 6.24 (t, J = 6.4 Hz, 1H), 6.06 (brs, 1H), 2.48 (s, 3H). | 359.1 |
| | 3-fluoro-2-methyl-N- | | | |
| | [3-(2-oxo-1,2- | | | |
| | dihydropyridin-3- | | | |
| | yl)phenyl]benzene- | | | |
| | sulfonamide | | | |

### Preparation of 7-((1-(3-fluoro-5-methylphenyl)-2-hydroxyethyl)amino)-3,4-dihydroisoquinolin-1(2H)-one (163)

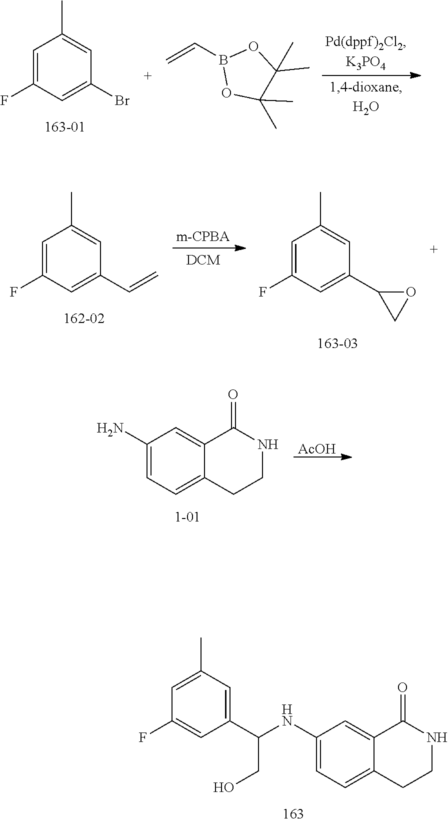

Step 1: A suspension of 4,4,5,5-tetramethyl-2-vinyl-1,3,2-dioxaborolane (509 mg, 2.12 mmol) and 1-bromo-3-fluoro-5-methylbenzene 163-01(1.00 g, 5.29 mmol) in 1,4-Dioxane (20 mL)/Water (4 mL) was prepared and argon was bubbled for 10 minutes. Pd(dppf)₂Cl₂ (193 mg, 0.265 mmol) and K₃PO₄ (3.36 g, 15.9 mmol) were added. The mixture was heated to 100° C. for 16 hrs under argon. The reaction was concentrated to dryness and the residue was taken up in EtOAc (20 mL×2) and the organics washed with 2×20 mL water then 20 mL of saturated brine solution. The organics were then separated and dried (MgSO₄) before concentration to dryness. The crude was then purified by flash column chromatography eluting 10% EtOAc in PE. The desired fractions were concentrated to dryness in vacuo to afford 1-fluoro-3-methyl-5-vinyl-benzene 163-02 (530 mg, 3.81 mmol, 72.1% yield) as a white oil.

Step 2: To a solution of 163-02 (150 mg, 1.10 mmol) in DCM (10 mL) was added 3-chlorobenzenecarboperoxoic acid (190 mg, 1.10 mmol). The reaction was stirred overnight at 25° C. The mixture was extracted by DCM (10 mL×3). The combined organic layers were washed with NaHSO₃ a.q. (10 mL×3), dried over Na₂SO₄ and concentrated to give the crude product, which was purified by silica gel chromatography (EA/PE=1:5) to give the desired product 2-(3-fluoro-5-methyl-phenyl) oxirane 163-03 (103 mg, 0.51 mmol, 46.1% yield) as a yellow oil. MS (m/z): 153.2 [M+H]⁺.

Step 3: To a solution of 163-03 (100 mg, 0.657 mmol) in Acetic acid (5 mL) was added 7-amino-3,4-dihydro-2H-isoquinolin-1-one 1-01 (107 mg, 0.657 mmol). The solution was stirred at room temperature for 12 h. The mixture was extracted by DCM (25 mL×3). The combined organic layers were washed with brine (15 mL×3), dried over Na₂SO₄ and concentrated, which was purified by prep-HPLC to give the desired product 7-[[1-(3-fluoro-5-methyl-phenyl)-2-hydroxy-ethyl]amino]-3,4-dihydro-2H-isoquinolin-1-one 163 (15 mg, 0.0466 mmol, 7.0% yield) as a yellow solid. MS (m/z): 315.1 [M+H]⁺. ¹H NMR (400 MHz, DMSO-d₆) δ 7.73 (d, J=2.8 Hz, 1H), 7.04 (s, 1H), 7.00 (d, J=2.4 Hz, 1H), 6.98-6.90 (m, 2H), 6.84 (d, J=9.7, 1H), 6.66 (dd, J=8.0, 2.4 Hz, 1H), 6.25 (d, J=6.4 Hz, 1H), 5.14 (s, 1H), 4.33 (q, J=6.0 Hz, 1H), 3.62-3.50 (m, 2H), 3.25 (t, J=6.8, 2H), 2.66 (t, J=6.8 Hz, 2H), 2.28 (s, 3H).

Compounds 238 and 250 were synthesized using a method like that used in Example 163.

| | | Appearance | | |
|---|---|---|---|---|
| Example | | and | | MS (m/z) |
| No. | Structure and Name | Yield | ¹H NMR Data | [M + H]⁺ |
| Example 238 Mixture of Stereoisomers | 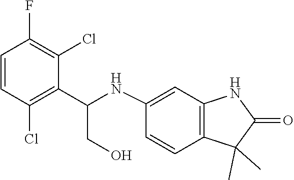 [SMILES: CC1(C)C(=O)Nc2cc(NC(CO)c3c(Cl)ccc(F)c3Cl)ccc21] | Yellow solid, yield: 35.8% | ¹H NMR (400 MHz, DMSO-d₆) δ 10.06 (br s, 1H), 7.52 (br s, 1H), 7.36 (t, J = 8.4 Hz, 1H), 6.86 (d, J = 8.0 Hz, 1H), 6.20-6.08 (m, 2H), 6.01 (dd, J = 8.1, 2.1 Hz, 1H), 5.16 (d, J = 7.4 Hz, 1H), 5.06 (t, J = 6.0 Hz, 1H), 3.97-3.80 (m, 2H), 1.11 (d, J = 3.8 Hz, 6H). | 383.2 |
| | 6-((1-(2,6-dichloro- | | | |
| | 3-fluorophenyl)-2- | | | |
| | hydroxyethyl)amino)- | | | |
| | 3,3-dimethylindolin- | | | |
| | 2-one | | | |
| Example 250 Mixture of Stereoisomers | 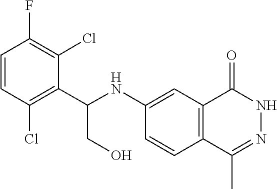 [SMILES: Cc1n[nH]c(=O)c2cc(NC(CO)c3c(Cl)ccc(F)c3Cl)ccc12] | White solid, yield: 41.3% | ¹H NMR (400 MHz, DMSO-d₆) δ 11.94 (s, 1H), 7.61 (d, J = 9.0 Hz, 1H), 7.38 (t, J = 8.5 Hz, 1H), 7.29 (d, J = 7.1 Hz, 1H), 7.13 (d, J = 8.7 Hz, 2H), 5.37- 5.29 (m, 1H), 4.05- 3.95 (m, 1H), 3.93- 3.85 (m, 1H), 3.13- 3.07 (m, 2H), 2.34 (s, 3H). | 382.3 |
| | 7-[[1-(2,6-dichloro- | | | |
| | 3-fluoro-phenyl)-2- | | | |
| | hydroxy- | | | |
| | ethyl]amino]-4- | | | |
| | methyl-2H- | | | |
| | phthalazin-1-one | | | |

### Preparation of 4-methyl-N-(1-oxo-1,2,3,4-tetrahydroisoquinolin-7-yl)piperidine-1-sulfonamide (164) Method 4

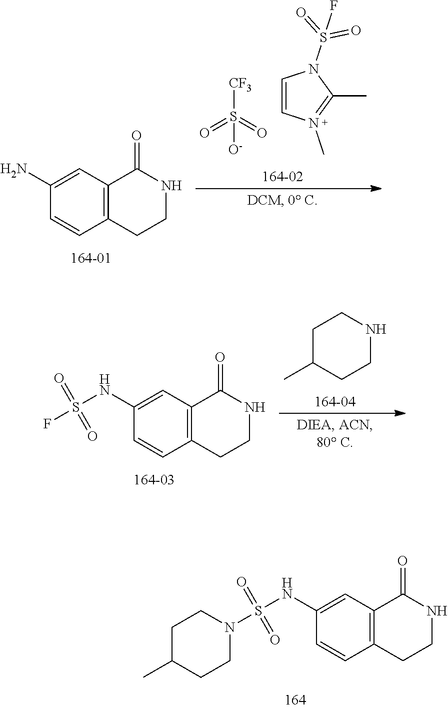

Step 1: A mixture solution of 7-amino-3,4-dihydroisoquinolin-1(2H)-one 164-01 (3 g, 0.019 mol), 1-(fluorosulfonyl)-2,3-dimethyl-1H-imidazol-3-ium trifluoromethanesulfonate 164-02 (6.073 g, 0.019 mol) and DCM (30 mL) was stirred at 0° C. for 16 hrs. After filtration, the filtrate was concentrated and the residue was purified through column chromatography on silica gel (EA/PE=1:1) to give the (1-oxo-1,2,3,4-tetrahydroisoquinolin-7-yl)sulfamoyl fluoride 164-03 (2.5 g, 55.56% yield) as a white solid. MS (ESI) m/z 245.1 [M+H]⁺.

Step 2: The mixture of (1-oxo-1,2,3,4-tetrahydroisoquinolin-7-yl)sulfamoyl fluoride (90 mg, 0.37 mmol), 4-methylpiperidine 164-04 (73 mg, 0.74 mmol) and DIEA (238 mg, 1.85 mmol) in ACN (3 mL) was stirred for 16 hour at 80° C. After reaction was completed, the solvent was removed under vacuum and the residue was purified by perp-HPLC (column-Gemini-C18 150×21.2 mm, 5 um; Mobile phase: ACN-H₂O (0.1% FA), 30%-50%) to afford the desired product 164 (7.7 mg) as a white solid. MS (ESI) m/z 324.1 [M+H]⁺. ¹H NMR (400 MHz, DMSO-d₆) δ 9.90 (s, 1H), 7.93 (s, 1H), 7.68 (d, J=2.2 Hz, 1H), 7.25 (dt, J=16.8, 5.4 Hz, 2H), 3.55 (d, J=12.2 Hz, 2H), 3.35 (d, J=3.0 Hz, 2H), 2.82 (t, J=6.6 Hz, 2H), 2.68-2.61 (m, 2H), 1.58 (d, J=10.6 Hz, 2H), 1.36 (s, 1H), 0.95 (dt, J=12.0, 8.6 Hz, 2H), 0.82 (d, J=6.6 Hz, 3H). Compounds 165-172 were synthesized using a method like that used in Example 164.

| | | Appear- | | |
|---|---|---|---|---|
| Example | | ance | | |
| No. | | and | | MS (m/z) |
| Method | Structure and Name | Yield | ¹H NMR Data | [M + H]⁺ |
| Example 165 Method 4 | 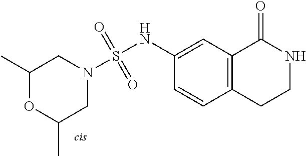 [SMILES: CC1CN(S(=O)(=O)Nc2ccc3c(c2)C(=O)NCC3)CC(C)O1] | White solid, Yield: 6.4% | ¹H NMR (400 MHz, DMSO-d₆) δ 10.03 (s, 1H), 7.95 (s, 1H), 7.69 (d, J = 2.2 Hz, 1H), 7.31-7.23 (m, 2H), 3.42 (d, J = 11.0 Hz, 4H), 3.34 (s, 2H), 2.83 (t, J = 6.4 Hz, 2H), 2.33 (s, 2H), 1.04 (d, J = 6.0 Hz, 6H). | 340.2 |
| | 2,6-dimethyl-N-(1- | | | |
| | oxo-1,2,3,4- | | | |
| | tetrahydroisoquinolin- | | | |
| | 7-yl)morpholine-4- | | | |
| | sulfonamide | | | |
| Example 166 Method 4 | 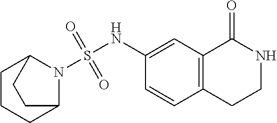 [SMILES: O=C1NCCc2ccc(NS(=O)(=O)N3C4CCCC3CC4)cc21] | White solid, Yield: 5.2% | ¹H NMR (400 MHz, DMSO-d₆) δ 9.92 (s, 1H), 7.92 (s, 1H), 7.71 (d, J = 2.2 Hz, 1H), 7.28-7.21 (m, 2H), 4.01 (s, 2H), 3.33 (td, J = 6.8, 2.6 Hz, 2H), 2.82 (t, J = 6.6 Hz, 2H), 1.82- | 336.1 |
| | N-(1-oxo-1,2,3,4- | | 1.75 (m, 2H), 1.64- | |
| | tetrahydroisoquinolin- | | 1.39 (m, 8H). | |
| | 7-yl)-8- | | | |
| | azabicyclo[3.2.1]octane- | | | |
| | 8-sulfonamide | | | |
| Example 167 Method 4 Mixture of Stereo- isomers | 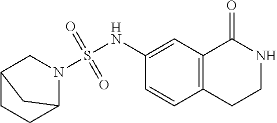 [SMILES: O=C1NCCc2ccc(NS(=O)(=O)N3CC4CCC3C4)cc21] | White solid, Yield: 6.1% | ¹H NMR (400 MHz, DMSO-d₆) δ 9.87 (s, 1H), 7.93 (s, 1H), 7.69 (d, J = 2.2 Hz, 1H), 7.27-7.21 (m, 2H), 3.99 (s, 1H), 3.33 (td, J = 6.6, 2.8 Hz, 2H), 3.11 (d, J = 8.4 Hz, 1H), | 322.1 |
| | N-(1-oxo-1,2,3,4- | | 2.84-2.80 (m, 3H), | |
| | tetrahydroisoquinolin- | | 1.54 (s, 3H), 1.31 (dt, J = | |
| | 7-yl)-2- | | 14.6, 7.6 Hz, 4H). | |
| | azabicyclo[2.2.1]hept- | | | |
| | ane-2-sulfonamide | | | |
| Example 168 Method 4 | 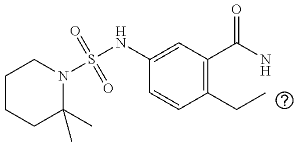 [SMILES: CCc1ccc(NS(=O)(=O)N2CCCCC2(C)C)cc1C(N)=O] | White solid, Yield: 5.1% | ¹H NMR (400 MHz, DMSO-d₆) δ 9.84 (s, 1H), 7.90 (s, 1H), 7.61 (d, J = 2.2 Hz, 1H), 7.23-7.12 (m, 2H), 3.33 (m, 4H), 2.82 (t, J = 6.8 Hz, 2H), 1.49- 1.20 (m, 12H). | 338.0 |
| | 2,2-dimethyl-N-(1- | | | |
| | oxo-1,2,3,4- | | | |
| | tetrahydroisoquinolin- | | | |
| | 7-yl)piperidine-1- | | | |
| | sulfonamide | | | |
| Example 169 Method 4 Mixture of Stereo- isomers | 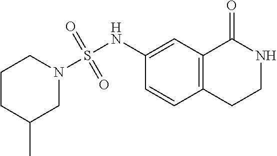 [SMILES: CC1CCCN(S(=O)(=O)Nc2ccc3c(c2)C(=O)NCC3)C1] | White solid, Yield: 6.7% | ¹H NMR (400 MHz, DMSO-d₆) δ 9.89 (s, 1H), 7.90 (s, 1H), 7.64 (d, J = 2.2 Hz, 1H), 7.23-7.17 (m, 2H), 3.45-3.40 (m, 2H), 3.28 (dd, J = 6.8, 2.8 Hz, 2H), 2.78 (t, J = 6.6 Hz, 2H), 2.56 (dd, J = 12.0, 2.6 Hz, 1H), 2.27 | 324.1 |
| | 3-methyl-N-(1-oxo- | | (d, J = 11.8 Hz, 1H), | |
| | 1,2,3,4- | | 1.59-1.25 (m, 4H), | |
| | tetrahydroisoquinolin- | | 0.90 (dd, J = 18.8, 7.4 | |
| | 7-yl)piperidine-1- | | Hz, 1H), 0.76 (d, J = | |
| | sulfonamide | | 6.8 Hz, 3H). | |
| Example 170 Method 4 Mixture of Stereoisomers | 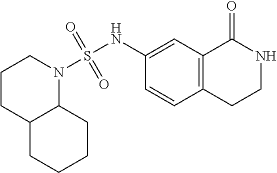 [SMILES: O=C1NCCc2ccc(NS(=O)(=O)N3CCCC4CCCCC43)cc21] | White solid, Yield: 4.8% | ¹H NMR (400 MHz, Chloroform-d) δ 7.80 (d, J = 2.5 Hz, 1H), 7.37 (dd, J = 8.2, 2.4 Hz, 1H), 7.18 (d, J = 8.2 Hz, 1H), 6.42 (s, 1H), 4.05 (dd, J = 13.1, 1.8 Hz, 1H), 3.57 (t, J = 6.1 Hz, 2H), 2.96 (t, J = 6.6 Hz, 2H), 2.94-2.84 (m, 1H), 2.76 (ddd, J = | 364.2 |
| | N-(1-oxo-1,2,3,4- | | 11.6, 10.2, 3.6 Hz, 1H), | |
| | tetrahydroisoquinolin- | | 2.39-2.28 (m, 1H), | |
| | 7- | | 1.79-1.58 (m, 7H), | |
| | yl)octahydroquinoline- | | 1.53-1.44 (m, 1H), | |
| | 1(2H)-sulfonamide | | 1.25-1.18 (m, 2H), | |
| | | | 1.14-0.89 (m, 2H). | |
| Example 171 Method 4 Mixture of Stereoisomers | 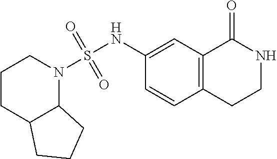 [SMILES: O=C1NCCc2ccc(NS(=O)(=O)N3CCCC4CCCC43)cc21] | White solid, Yield: 3.9% | ¹H NMR (400 MHz, Chloroform-d) δ 7.81 (d, J = 2.4 Hz, 1H), 7.39 (dd, J = 8.2, 2.4 Hz, 1H), 7.17 (d, J = 8.2 Hz, 1H), 6.39 (s, 1H), 4.19-4.07 (m, 1H), 3.67-3.50 (m, 3H), 3.04-2.80 (m, 3H), 1.91-1.86 (m, | 350.1 |
| | N-oxo-1,2,3,4- | | 1H), 1.77-1.45 (m, | |
| | tetrahydroisoquinolin- | | 7H), 1.37-1.24 (m, | |
| | 7-yl)octahydro-1H- | | 2H), 1.12 (qd, J = 13.0, | |
| | cyclopenta[b]pyridine- | | 3.6 Hz, 1H). | |
| | 1-sulfonamide | | | |
| Example 172 Method 4 Mixture of Stereoisomers | 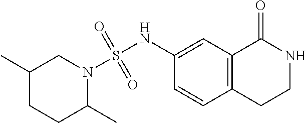 [SMILES: CC1CCC(C)N(S(=O)(=O)Nc2ccc3c(c2)C(=O)NCC3)C1] | White solid, Yield: 7.4% | ¹H NMR (400 MHz, DMSO-d₆) δ 9.90 (s, 1H), 7.92 (s, 1H), 7.63 (t, J = 2.2 Hz, 1H), 7.25- 7.13 (m, 2H), 4.00- 3.91 (m, 1H), 3.39 (d, J = 3.8 Hz, 0.35H), 3.33 (m, 2H), 3.17 (m, | 338.0 |
| | 2,5-dimethyl-N-(1- | | 0.45H), 3.02 (d, J = | |
| | oxo-1,2,3,4- | | 12.6 Hz, 0.42H), 2.82 | |
| | tetrahydroisoquinolin- | | (m, J = 8.6, 4.2 Hz, | |
| | 7-yl)piperidine-1- | | 2H), 2.54 (d, J = 12.8 | |
| | sulfonamide | | Hz, 0.46H), 1.75 (m, | |
| | | | 1.21H), 1.42 (t, J = 12.4 | |
| | | | Hz, 1.79H), 1.26-1.02 | |
| | | | (m, 5.06H), 0.79 (d, J = | |
| | | | 6.4 Hz, 1.80H), 0.62 (d, | |
| | | | J = 6.8 Hz, 1.20H). | |

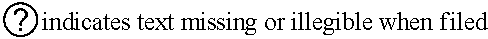

### Preparation of 2-(3-fluoro-5-methylphenyl)-2-((1-oxo-1,2,3,4-tetrahydroisoquinolin-7-yl)amino)acetonitrile (173)

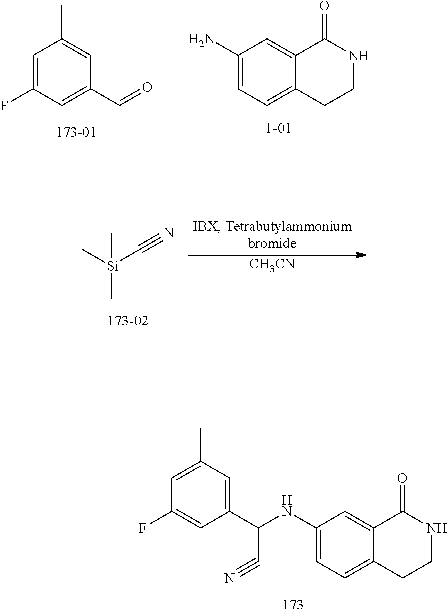

To a solution of 3-Fluoro-5-methylbenzaldehyde 173-01 (100 mg, 0.72 mmol) and 7-amino-3,4-dihydro-2H-isoquinolin-1-one 1-01 (117 mg, 0.724 mmol) and trimethylsilanecarbonitrile 173-02 (79 mg, 0.796 mmol) in MeCN (10 mL), tetrabutylammonium bromide (257 mg, 0.80 mmol) and IBX (223 mg, 0.80 mmol) were added. The mixture was stirred at room temperature for 16 h. The mixture was extracted by EA (25 mL×3). The combined organic layers were washed with brine (35 mL×3), dried over Na₂SO₄ and concentrated to give the crude product, which was purified by prep HPLC to give the desired product 2-(3-fluoro-5-methyl-phenyl)-2-[(1-oxo-3,4-dihydro-2H-isoquinolin-7-yl)amino]acetonitrile 173 (14 mg, 0.043 mmol, 6.0% yield) as a white solid. MS (m/z): 357.3 [M+H]⁺, ¹H NMR (400 MHz, DMSO-d₆) δ 7.85 (d, J=2.8 Hz, 1H), 7.34 (d, J=2.4 Hz, 1H), 7.28 (s, 1H), 7.19 (d, J=9.6, 1H), 7.15-7.08 (m, 2H), 6.94 (dd, J=8.0, 2.8 Hz, 1H), 6.78 (d, J=9.6 Hz, 1H), 6.04 (d, J=9.6 Hz, 1H), 3.30 (d, J=2.8 Hz, 2H), 2.77 (t, J=6.4 Hz, 2H), 2.37 (s, 3H).

### Preparation of 7-((2,2,2-trifluoro-1-(3-fluoro-5-methylphenyl)ethyl)amino)-3,4-dihydroisoquinolin-1(2H)-one (174)

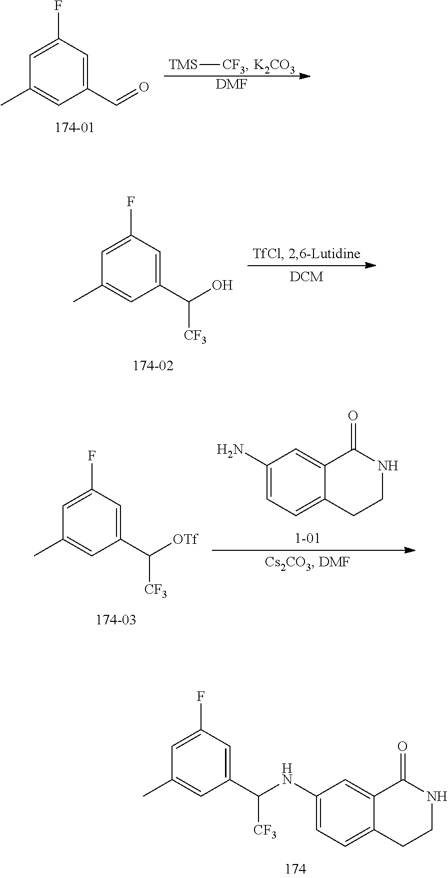

Step 1: To a solution of 174-01 (200 mg, 1.45 mmol) and TMS-CF₃ (618 mg, 4.34 mmol) in DMF (5 mL) was added potassium carbonate (20 mg, 0.145 mmol) and the reaction mixture was stirred at 25° C. overnight. The reaction progress was monitored by TLC. Once completed, water (10 mL) was added to the mixture slowly to quench the reaction. The reaction mixture was extracted with DCM, dried (MgSO₄) and concentrated under reduced pressure. The residue was purified by silica column chromatograph (PE:EA=10:1) to give the product (292 mg, 97%).

¹H NMR (400 MHz, Chloroform-d) δ 7.05 (qd, J=1.7, 0.9 Hz, 1H), 7.02-6.95 (m, 1H), 6.91 (dddd, J=9.5, 2.4, 1.6, 0.8 Hz, 1H), 4.94 (q, J=6.7 Hz, 1H), 3.37 (bs, 11H).

Step 2: To a solution of 174-02 (250 mg, 1.20 mmol) in DCM (5 mL) were added 2,6-dimethylpyridine (0.42 mL, 2.40 mmol) and trifluoromethylsulfonic anhydride (0.12 mL, 1.80 mmol) at 0° C. Then the reaction mixture was stirred at 25° C. for 2 h. The reaction progress was monitored by TLC. The SM disappeared and a new spot was found. The solvent was removed under reduced pressure and the crude was used directly in the next step.

Step 3: To a solution of 174-03 (408 mg, 1.20 mmol) in DMF (5 mL) were added 1-01 (584 mg, 3.60 mmol) and cesium carbonate (1.17 g, 3.60 mmol). Then the reaction mixture was stirred at 25° C. weekends.

The reaction progress was monitored by LC/MS. Desired mass was found with a tiny peak. The mixture was diluted with water and extracted with EA. The organic layer was dried and concentrated under reduced pressure. The residue was purified by prep-HPLC to yield the product 174 (1 mg, 0.2%). MS (m/z): 353.02 [M+H]⁺. ¹H NMR (400 MHz, Chloroform-d) δ 7.78 (s, 1H), 7.34 (d, J=2.4 Hz, 1H), 7.31-7.27 (m, 2H), 7.04-7.01 (m, 2H), 6.95 (dd, J=8.4, 2.4 Hz, 1H), 6.73 (d, J=10.4 Hz, 1H), 5.74-5.44 (m, 1H), 3.28-3.26 (m, 2H), 2.70 (t, J=6.4 Hz, 2H), 2.30 (m, 2H).

### Preparation of 7-((3-(3-fluoro-5-methylphenyl)oxetan-3-yl)amino)-2-methyl-3,4-dihydroisoquinolin-1(2H)-one (175)

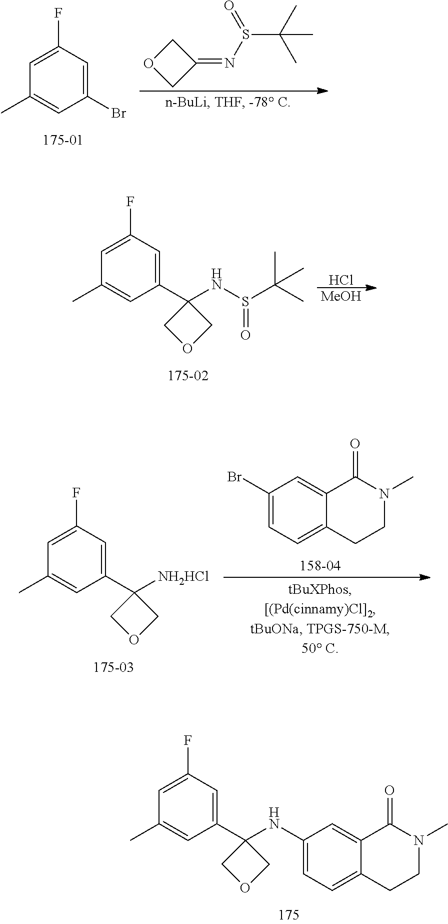

Step 1: To a solution of 175-01 (500 mg, 2.65 mmol) in THF (15 mL) was added n-BuLi (2.4 M in hexanes, 1.2 mL, 2.91 mmol) at −70° C. and the reaction mixture was stirred for 20 min. Then 2-methyl-N-(oxetan-3-ylidene)propane-2-sulfinamide (5.00 eq, 2.3 g, 13.2 mmol) was added at −70° C. and the mixture was allowed to warm to rt and stirred for another 2 h. The reaction progress was monitored by LC/MS. The desired mass was found in main peak. The reaction mixture was diluted with water (50 mL) and extracted with DCM (100 mL, 2 times). The organic layer was separated, dried (MgSO₄) and concentrated under reduced pressure. The residue was purified by silica column chromatograph (PE:EA=5:1) to give the product 175-02 (480 mg, 64%). MS (m/z) 286.24 [M+H]⁺

Step 2: To a solution of 175-02 (220 mg, 0.77 mmol) in methanol (1.5 mL) was added HCl (4M in dioxane, 0.35 mL, 1.16 mmol) at 0° C. and the reaction mixture was stirred for 10 min. The reaction progress was monitored by LC/MS. The product was identified. Then the solvent was removed and the obtained solid was washed with MTBE, dried to yield the product (52 mg, 37%). ¹H NMR (400 MHz, DMSO-d₆) δ 9.31 (s, 2H), 7.32-7.18 (m, 2H), 7.17-7.05 (m, 1H), 4.97 (d, J=7.5 Hz, 2H), 4.88 (d, J=7.5 Hz, 2H), 2.37 (s, 3H).

Step 3: To a mixture of 175-03 (5.7 mg, 0.0316 mmol) and 175-04 (7.6 mg, 0.0316 mmol) were added t-BuONa (9.1 mg, 0.0948 mmol), tBuXPhos (1.3 mg, 0.0032 mmol), [Pd(cinnamyl)Cl]₂ (0.82 mg, 0.0016 mmol) and TPGS-750-M (1.0 mL). Then the mixture was stirred at 50° C. overnight. The reaction progress was monitored by LC-MS. Desired mass was found. Then the reaction mixture was diluted with water (10 mL), extracted with DCM (15 mL, 2 times). The organic layer was separated, dried (MgSO₄) and concentrated under reduced pressure. The residue was purified by prep-HPLC to yield the product 175 (5 mg, 46%). MS (m/z) 315.29 [M+H]⁺. ¹H NMR (400 MHz, DMSO-d₆) δ 7.34 (s, 1H), 7.10-7.04 (m, 2H), 6.96-6.90 (m, 2H), 6.80 (d, J=2.4 Hz, 1H), 6.26 (dd, J=8.4, 2.4 Hz, 1H), 4.83 (d, J=6.0 Hz, 2H), 4.75 (d, J=6.0 Hz, 2H), 3.43 (t, J=6.4 Hz, 2H), 2.95 (s, 3H), 2.75 (t, J=6.4 Hz, 2H), 2.33 (s, 3H).

### Preparation of 7-(1-(3-fluoro-5-methylphenyl)-2-hydroxyethoxy)-3,4-dihydroisoquinolin-1(2H)-one (176)

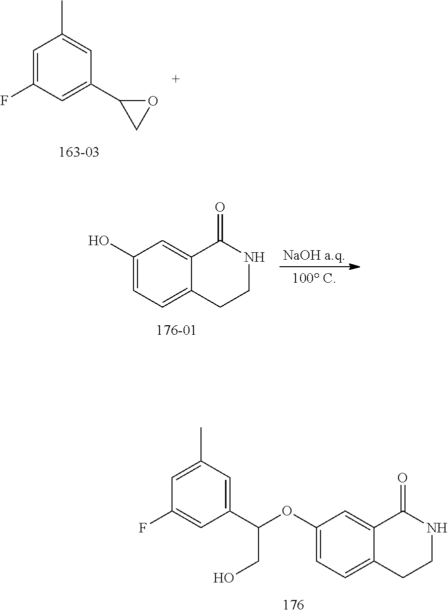 [SMILES: Cc1cc(F)cc(C2CO2)c1 ; O=C1NCCc2ccc(O)cc21 ; Cc1cc(F)cc(C(CO)Oc2ccc3c(c2)C(=O)NCC3)c1]

Step 1: To a solution of 2-(3-fluoro-5-methyl-phenyl)oxirane 163-03 (100 mg, 0.657 mmol) in NaOH a.q. (5 mL) was added 7-hydroxy-3,4-dihydro-2H-isoquinolin-1-one 176-01 (107 mg, 0.66 mmol). The reaction was stirred overnight at 100° C. The mixture was extracted by EA (10 mL×3). The combined organic layers were washed with brine (10 mL×3), dried over Na₂SO₄ and concentrated to give the crude product, which was purified by prep-HPLC to give the desired product 7-[1-(3-fluoro-5-methyl-phenyl)-2-hydroxy-ethoxy]-3,4-dihydro-2H-isoquinolin-1-one 176 (11 mg, 0.034 mmol, 5.1% yield) as a white solid. MS (m/z): 316.3 [M+H]⁺, ¹H NMR (400 MHz, DMSO-d₆) δ 7.90 (d, J=2.8 Hz, 1H), 7.32 (d, J=2.8 Hz, 1H), 7.16 (d, J=8.4 Hz, 1H), 7.08-7.02 (m, 2H), 7.00 (d, J=9.6, 1H), 6.92 (d, J=10.0 1H), 5.30-5.25 (m, 1H), 5.14 (t, J=6.0 Hz, 1H), 3.77-3.55 (m, 2H), 3.31-3.27 (m, 2H), 2.77 (t, J=6.5 Hz, 2H), 2.29 (s, 3H).

### Preparation of 7-(((3-fluoro-5-methylphenyl)(pyridin-4-yl)methyl)amino)-3,4-dihydroisoquinolin-1(2H)-one (177) Method 5

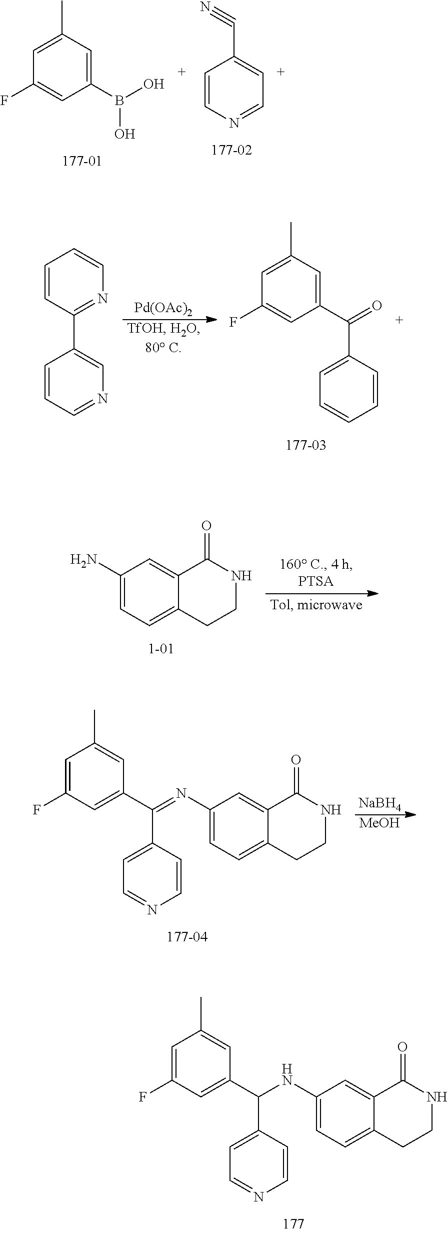

Step 1: To a solution of (3-fluoro-5-methyl-phenyl)boronic acid 177-01 (532 mg, 3.46 mmol) and pyridine-4-carbonitrile 177-02 (300 mg, 2.88 mmol) in Water (9 mL) and TfOH (3 mL) were added 2-(2-pyridyl)pyridine (23 mg, 0.144 mmol) and Pd(OAc)₂ (32 mg, 0.144 mmol). The mixture was stirred at 80° C. for 16 h. The mixture was extracted by EA (25 mL×3). The combined organic layers were washed with brine (15 mL×3), dried over Na₂SO₄ and concentrated to give the crude product, which was purified by TLC (MeOH/DCM=1/25) to give the desired product (3-fluoro-5-methyl-phenyl)-(4-pyridyl)methanone 177-03 (285 mg. 1.26 mmol, 43.6% yield) as a yellow oil. MS (m/z): 216.2 [M+H]⁺.

Step 2: A solution of 177-03 (50 mg, 0.23 mmol), 7-amino-3,4-dihydro-2H-isoquinolin-1-one 1-01 (38 mg, 0.23 mmol), p-Toluenesulfonic acid (4.0 mg, 0.023 mmol) in Toluene (5 mL) was stirred at 160° C. under microwave for 5 h. The reaction was filtered and concentrated under vacuum to afford the desired product 7-[(E)-[(3-fluoro-5-methyl-phenyl)-(4-pyridyl)methylene]amino]-3,4-dihydro-2H-isoquinolin-1-one 177-04 (45 mg, 0.075 mmol, 32.34% yield) as yellow oil. MS (m/z): 360.3 [M+H]⁺.

Step 3: To a solution of 177-04 (45 mg, 0.125 mmol) in Methanol (5 mL) was added NaBH₄ (9.5 mg, 0.25 mmol). The solution was stirred at 25° C. for 1 h. The reaction was quenched with water (10 mL), and extracted by DCM (15 ml×3). The combined organic layers were washed with brine (15 mL×3), dried over sodium sulfate and concentrated to give the crude product, which was purified by prep-HPLC to give the desired product 7-[[(3-fluoro-5-methyl-phenyl)-(4-pyridyl)methyl]amino]-3,4-dihydro-2H-isoquinolin-1-one 177 (6.0 mg, 0.016 mmol, 13.1% yield) as a white solid. MS (m/z): 362.1 [M+H]⁺. ¹H NMR (400 MHz, DMSO-d₆) δ 8.51 (d, J=5.2 Hz, 2H), 7.74 (d, J=2.8 Hz, 1H), 7.47-7.41 (m, 2H), 7.18 (d, J=2.8 Hz, 1H), 7.13 (s, 1H), 7.10 (d, J=9.8 Hz, 1H), 6.98 (d, J=8.0 Hz, 1H), 6.91 (d, J=9.6, 1H), 6.81 (dd, J=8.2, 2.6 Hz, 1H), 6.64 (d, J=8.0 Hz, 1H), 5.73 (d, J=7.6 Hz, 1H), 3.28-3.25 (m, 2H), 2.69 (t, J=6.8 Hz, 2H), 2.28 (s, 3H).

Compound 178 was synthesized using a method similar to that used for Compound 177.

| | | Appear- | | |
|---|---|---|---|---|
| Example | | ance | | |
| No. | | and | | MS (m/z) |
| Method | Structure and Name | Yield | ¹H NMR Data | [M + H]⁺ |
| Example 178 Method 5 Mixture of Stereoisomers | 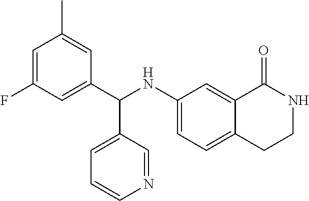 [SMILES: Cc1cc(F)cc(C(Nc2ccc3c(c2)C(=O)NCC3)c2cccnc2)c1] | White solid, Yield: 14.57% | ¹H NMR (400 MHz, DMSO-d₆) δ 8.67 (s, H), 8.46 (s, 1H), 7.81 (d, J = 8.0 Hz, 1H), 7.74 (d, J = 2.8 Hz, 1H), 7.42- 7.35 (m, 1H), 7.19 (d, J = 2.8 Hz, 1H), 7.13 (s, 1H), 7.09 (d, J = 9.8, 1H), 6.99 (d, J = 8.0 Hz, 1H), 6.90 (d, J = 9.6, 1H), 6.82 (dd, J = 8.2, 2.6 Hz, 1H), 6.63 (d, J = | 362.1 |
| | 7-(((3-fluoro-5- | | 7.6 Hz, 1H), 5.78 (d, J = | |
| | methylphenyl)(pyridin- | | 7.2 Hz, 1H), 3.29-3.23 | |
| | 3- | | (m, 2H), 2.69 (t, J = 6.4 | |
| | yl)methyl)amino)- | | Hz, 2H), 2.28 (s, 3H). | |
| | 3,4- | | | |
| | dihydroisoquinolin- | | | |
| | 1(2H)-one | | | |

### Preparation of N-(cyclobutyl(2,3-dichlorophenyl)methyl)quinolin-7-amine (179)

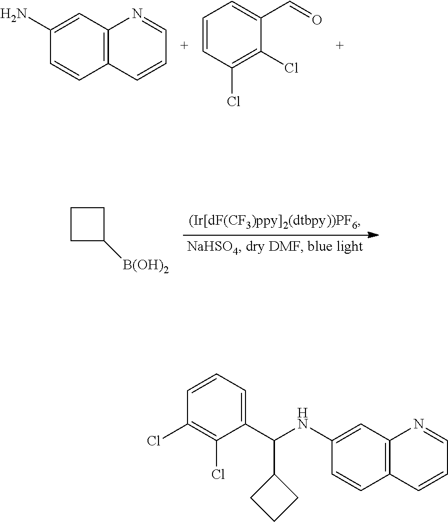

A mixture of quinolin-7-amine (1.00 eq, 150 mg, 1.04 mmol), (Ir[dF(CF₃)ppy]₂(dtbpy))PF₆ (0.0500 eq, 58 mg, 0.0520 mmol)cyclobutylboronic acid (2.00 eq, 208 mg, 2.08 mmol), NaHSO₄ (0.200 eq, 25 mg, 0.208 mmol) and 2,3-dichlorobenzaldehyde (1.00 eq, 182 mg, 1.04 mmol) in dry DMF (10 mL) was stirred for 2 days at 45° C. under blue light under nitrogen. The reaction mixture was diluted with water and extracted with ethyl acetate twice. The combined organic layers were washed with water and brine, dried over sodium sulfate and concentrated under vacuum. The residue was purified by Prep-TLC (dichloromethane/methanol 10/1) and then purified by Prep-HPLC to afford N-[cyclobutyl-(2,3-dichlorophenyl)methyl]quinolin-7-amine (13 mg, 0.0353 mmol, 3.3% yield) as a yellow solid. ¹H NMR (400 MHz, DMSO-d₆) δ 8.55 (dd, J=4.3, 1.8 Hz, 1H), 7.98 (dd, J=8.0, 1.8 Hz, 1H), 7.60 (d, J=8.9 Hz, 1H), 7.53-7.42 (m, 2H), 7.30 (t, J=7.8 Hz, 1H), 7.19-7.11 (m, 1H), 7.07 (dd, J=8.0, 4.3 Hz, 1H), 6.94 (d, J=7.4 Hz, 1H), 6.55 (d, J=2.3 Hz, 1H), 4.92 (t, J=8.1 Hz, 1H), 2.78-2.66 (m, 1H), 2.17-2.06 (m, 1H), 2.02-1.90 (m, 2H), 1.86-1.79 (m, 2H), 1.78-1.70 (m, 1H).

### Preparation of N-(cyclopropyl(2,3-dichlorophenyl)methyl)quinolin-7-amine (180)

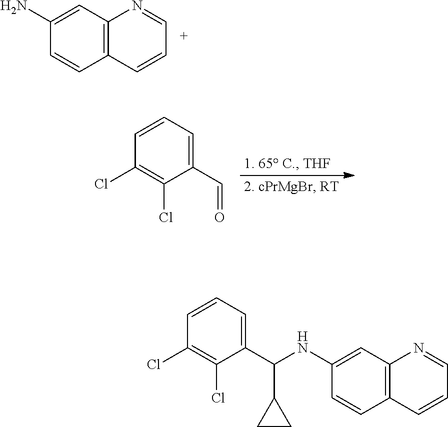 [SMILES: Nc1ccc2cccnc2c1 ; O=Cc1cccc(Cl)c1Cl ; Clc1cccc(C(Nc2ccc3cccnc3c2)C2CC2)c1Cl]

A mixture of quinolin-7-amine (1.00 eq, 144 mg, 0.999 mmol) and 2,3-dichlorobenzaldehyde (1.00 eq, 175 mg, 0.999 mmol) in THF (10 mL) was stirred for 2 h at 65° C. The reaction mixture was cooled before bromo(cyclopropyl)magnesium (0.5 M in THF) (3.00 eq, 6.0 mL, 3.00 mmol) was added. The reaction was stirred overnight at RT and then quenched with water. The resulting mixture was extracted with ethyl acetate twice. The combined organic layers were washed with brine, dried over sodium sulfate and concentrated under vacuum. The residue was purified by Prep-TLC (dichloromethane/methanol 15/1) and then Prep-HPLC to afford N-[cyclopropyl-(2,3-dichlorophenyl)methyl]quinolin-7-amine (19 mg, 0.0548 mmol, 5.4% yield) as a yellow solid. LCMS (ESI, m/z) 343.2 [M+H]⁺. ¹H NMR (400 MHz, DMSO-d₆) δ 8.53 (dd, J=4.3, 1.8 Hz, 1H), 7.98 (dd, J=8.0, 1.8 Hz, 1H), 7.60 (d, J=8.9 Hz, 1H), 7.57-7.49 (m, 2H), 7.32 (t, J=7.9 Hz, 1H), 7.22 (d, J=6.9 Hz, 1H), 7.15 (dd, J=8.9, 2.4 Hz, 1H), 7.07 (dd, J=8.0, 4.3 Hz, 1H), 6.44 (d, J=2.2 Hz, 1H), 4.48 (t, J=7.5 Hz, 1H), 1.33-1.23 (m, 1H), 0.68-0.56 (m, 1H), 0.56-0.45 (m, 2H), 0.41-0.29 (m, 1H).

### Preparation of 7-((1-(2,3-dichlorophenyl)ethyl)amino)-1,4-dihydro-2H-benzo[d][1,3]oxazin-2-one (181) Method 6

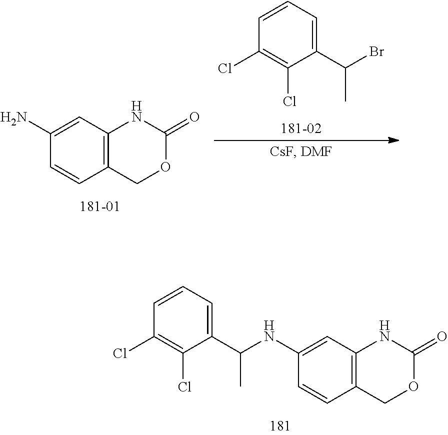 [SMILES: CC(Br)c1cccc(Cl)c1Cl ; Nc1ccc2c(c1)NC(=O)OC2 ; CC(Nc1ccc2c(c1)NC(=O)OC2)c1cccc(Cl)c1Cl]

181-1 (100 mg, 0.61 mmol), 181-02 (307 mg, 1.22 mmol) and CsF (185 mg, 1.21 mmol) were dissolved in DMF (5 mL) and stirred at 30° C. for 4 h. The mixture was extracted with EA, washed with brine, dried (Na₂SO₄), and concentrated in vacuo. Purification by Pre-HPLC gave the titled compound 181 (4.7 mg, 2.3%) as a light green solid. MS (m/z): 337.0 (M+H+), ¹H NMR (400 MHz, Methanol-d₄) δ 10.71 (s, 1H), 8.31 (dd, J=7.8, 1.8 Hz, 1H), 8.19 (dd, J=7.8, 1.8 Hz, 1H), 8.11 (t, J=7.8 Hz, 1H), 7.62 (d, J=8.1 Hz, 1H), 7.43 (d, J=7.0 Hz, 1H), 6.86 (dd, J=8.2, 2.2 Hz, 1H), 6.79 (d, J=2.2 Hz, 1H), 5.84 (s, 2H), 5.55 (p, J=6.8 Hz, 1H), 2.22 (d, J=6.6 Hz, 3H).

Compounds 182-186, 215, 229-231, 233-234A, 236, 245-246, 249, 257-263, 266-267, 271, 275, 277-278, 282, 28, 302-302C, 310-311, 314-316, 318, 321-322, 324-325, 330, 342, 349, 350-354, 382-388 were synthesized using a method like that used for Compound 181. Some other substitution conditions were also used according to different substrate:

2) DMF, H₂O (1/1), KI, CaCO₃; 3) DMF, KI, CaCO₃; or4) DMF, KI, CsCO₃

| | | Appear- | | |
|---|---|---|---|---|
| Example | | ance | | |
| No. | | and | | MS (m/z) |
| Method | Structure and Name | Yield | ¹H NMR Data | [M + H]⁺ |
| Example 182 Method 6 Mixture of Stereo- isomers | 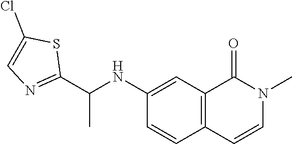 [SMILES: CC(Nc1ccc2ccn(C)c(=O)c2c1)c1ncc(Cl)s1] | Yellow solid, yield: 29.0% | ¹H NMR (400 MHz, DMSO-d₆) δ 7.75 (s, 1H), 7.42 (d, J = 8.6 Hz, 1H), 7.23 (d, J = 2.6 Hz, 1H), 7.16 (d, J = 7.2 Hz, 1H), 7.11 (dd, J = 8.6, 2.6 Hz, 1H), 6.91 (d, J = 6.0 Hz, 1H), 6.43 (d, J = 7.2 Hz, 1H), 4.81 (p, J = | 320 |
| | 7-((1-(5- | | 6.6 Hz, 1H), 3.43 (s, | |
| | chlorothiazol-2- | | 3H), 1.57 (d, J = 6.8 Hz, | |
| | yl)ethyl)amino)-2- | | 3H). | |
| | methylisoquinolin- | | | |
| | 1(2H)-one | | | |
| Example 183 Method 6 Mixture of Stereo- isomers | 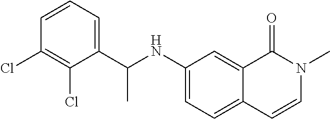 [SMILES: CC(Nc1ccc2ccn(C)c(=O)c2c1)c1cccc(Cl)c1Cl] | white solid, yield: 42.1% | ¹H NMR (400 MHz, DMSO-d₆) δ 7.50 (dd, J = 7.6, 1.6 Hz, 1H), 7.41 (dd, J = 7.8, 1.6 Hz, 1H), 7.35 (d, J = 9.0 Hz, 1H), 7.28 (t, J = 7.8 Hz, 1H), 7.10 (d, J = 7.2 Hz, 1H), 7.02-6.97 | 347.3 |
| | 7-((1-(2,3- | | (m, 2H), 6.91 (d, J = | |
| | dichlorophenyl)ethyl) | | 7.0 Hz, 1H), 6.37 (d, J = | |
| | amino)-2- | | 7.2 Hz, 1H), 4.90 (p, | |
| | methylisoquinolin- | | J = 6.8 Hz, 1H), 3.40 (s, | |
| | 1(2H)-one | | 3H), 1.46 (d, J = 6.8 | |
| | | | Hz, 3H). | |
| Example 184 Method 6 Mixture of Stereo- isomers | 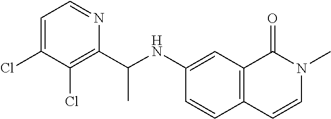 [SMILES: CC(Nc1ccc2ccn(C)c(=O)c2c1)c1nccc(Cl)c1Cl] | white solid, yield: 32.1% | ¹H NMR (400 MHz, DMSO-d₆) δ 8.64 (s, 1H), 8.52 (s, 1H), 7.38 (d, J = 8.8 Hz, 1H), 7.12 (d, J = 7.2 Hz, 1H), 7.06-7.01 (m, 2H), 6.94 (d, J = 7.2 Hz, 1H), 6.39 (d, J = | 348.3 |
| | 7-((1-(4,5- | | 7.3 Hz, 1H), 4.93 (p, J = | |
| | dichloropyridin-3- | | 6.7 Hz, 1H), 3.40 (s, | |
| | yl)ethyl)amino)-2- | | 3H), 1.53 (d, J = 6.7 | |
| | methylisoquinolin- | | Hz, 3H). | |
| | 1(2H)-one | | | |
| Example 185 Method 6 Mixture of Stereo- isomers | 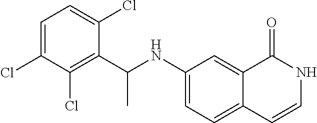 [SMILES: CC(Nc1ccc2cc[nH]c(=O)c2c1)c1c(Cl)ccc(Cl)c1Cl] | yellow solid, yield: 6.7% | ¹H NMR (400 MHz, DMSO-d₆) δ 10.81 (d, J = 5.6 Hz, 1H), 7.54 (d, J = 8.2 Hz, 1H), 7.33 (d, J = 8.6 Hz, 1H), 7.08 (d, J = 2.5 Hz, 1H), 7.00 (dd, J = 8.6, 2.6 Hz, 1H), 6.82-6.74 | 367 |
| | 7-((1-(2,3,6- | | (m, 2H), 6.29 (d, J = | |
| | trichlorophenyl)ethyl) | | 7.0 Hz, 1H), 5.42-5.27 | |
| | amino)isoquinolin- | | (m, 1H), 1.61 (d, J = | |
| | 1(2H)-one | | 6.9 Hz, 3H). | |
| Example 186 Method 6 Mixture of Stereo- isomers | 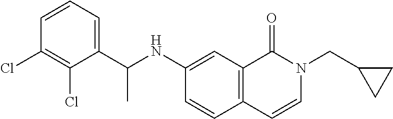 [SMILES: CC(Nc1ccc2ccn(CC3CC3)c(=O)c2c1)c1cccc(Cl)c1Cl] | white solid, yield: 45.3% | ¹H NMR (400 MHz, DMSO-d₆) δ 7.50 (dd, J = 8.0, 1.6 Hz, 1H), 7.42 (dd, J = 7.8, 1.6 Hz, 1H), 7.36 (d, J = 8.4 Hz, 1H), 7.29 (t, J = 7.8 Hz, 1H), 7.14 (d, J = 7.2 Hz, 1H), 7.04-6.98 | 387.1 |
| | 2- | | (m, 2H), 6.91 (d, J = | |
| | (cyclopropylmethyl)- | | 7.0 Hz, 1H), 6.39 (d, J = | |
| | 7-((1-(2,3- | | 7.2 Hz, 1H), 4.91 (p, | |
| | dichlorophenyl)ethyl) | | J = 6.6 Hz, 1H), 3.72 | |
| | amino)isoquinolin- | | (dd, J = 7.2, 3.8 Hz, | |
| | 1(2H)-one | | 2H), 1.47 (d, J = 6.6 | |
| | | | Hz, 3H), 1.24-1.12 | |
| | | | (m, 1H), 0.46-0.39 | |
| | | | (m, 2H), 0.37-0.31 | |
| | | | (m, 2H). | |
| Example 215 Method 6 Mixture of Stereo- isomers | 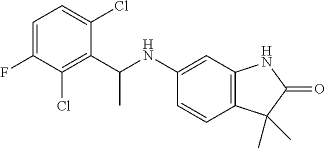 [SMILES: CC(Nc1ccc2c(c1)NC(=O)C2(C)C)c1c(Cl)ccc(F)c1Cl] | red solid, yield: 41.1% | ¹H NMR (400 MHz, DMSO-d₆) δ 10.06 (s, 1H), 7.44 (s, 1H), 7.33 (t, J = 8.6 Hz, 1H), 6.84 (d, J = 8.0 Hz, 1H), 6.31 (d, J = 7.8 Hz, 1H), 6.09 (d, J = 2.1 Hz, 1H), 5.96 (dd, J = 8.1, 2.1 Hz, 1H), 5.19- 5.10 (m, 1H), 1.55 (d, J = | 367.07 |
| | 6-[1-(2,6-dichloro-3- | | 7.0 Hz, 3H), 1.11 (s, | |
| | fluoro- | | 3H), 1.10 (s, 3H). | |
| | phenyl)ethylamino]- | | | |
| | 3,3-dimethyl-indolin- | | | |
| | 2-one | | | |
| Example 229 Method 6 Mixture of Stereo- isomers | 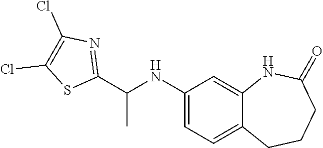 [SMILES: CC(Nc1ccc2c(c1)NC(=O)CCC2)c1nc(Cl)c(Cl)s1] | white solid, yield: 30.4% | ¹H NMR (400 MHz, DMSO-d₆) δ 9.34 (s, 1H), 6.95 (d, J = 8.2 Hz, 1H), 6.51 (d, J = 5.5 Hz, 1H), 6.32 (dd, J = 8.1, 2.4 Hz, 1H), 6.25 (d, J = 2.4 Hz, 1H), 4.67-4.59 (m, 1H), 2.55-2.51 (m, 2H), 2.15-2.06 (m, 2H), | 356.03 |
| | 8-[1-(4,5- | | 2.05-1.94 (m, 2H), | |
| | dichlorothiazol-2- | | 1.51 (d, J = 6.8 Hz, | |
| | yl)ethylamino]- | | 3H). | |
| | 1,3,4,5-tetrahydro-1- | | | |
| | benzazepin-2-one | | | |
| Example 230 Method 6 Mixture of Stereo- isomers | 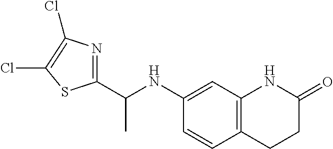 [SMILES: CC(Nc1ccc2c(c1)NC(=O)CC2)c1nc(Cl)c(Cl)s1] | White solid, yield: 23.7% | ¹H NMR (400 MHz, DMSO-d₆) δ 9.86 (s, 1H), 6.87 (d, J = 8.0 Hz, 1H), 6.48 (d, J = 5.5 Hz, 1H), 6.21-6.13 (m, 2H), 4.63-4.55 (m, 1H), 2.69 (t, J = 8.5, 6.5 Hz, 2H), 2.36 (t, J = 8.5, 6.5 Hz, 2H), 1.50 (d, J = 6.8 Hz, | 341.92 |
| | 7-[1-(4,5- | | 3H). | |
| | dichlorothiazol-2- | | | |
| | yl)ethylamino]-3,4- | | | |
| | dihydro-1H-quinolin- | | | |
| | 2-one | | | |
| Example 231 Method 6 Mixture of Stereo- isomers | 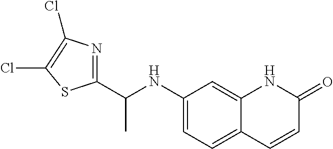 [SMILES: CC(Nc1ccc2ccc(=O)[nH]c2c1)c1nc(Cl)c(Cl)s1] | White solid, yield: 27.8% | ¹H NMR (400 MHz, DMSO-d₆) δ 11.29 (s, 1H), 7.64 (dd, J = 9.4, 1.9 Hz, 1H), 7.35 (dd, J = 8.6, 1.9 Hz, 1H), 7.17 (dd, J = 5.9, 1.9 Hz, 1H), 6.59-6.53 (m, 1H), 6.37 (d, J = 2.2 Hz, 1H), 6.10 (d, J = 10.1, 2.5 Hz, 1H), 4.81- | 339.92 |
| | 7-[1-(4,5- | | 4.71 (m, 1H), 1.56 | |
| | dichlorothiazol-2- | | (dd, J = 6.8, 1.9 Hz, | |
| | yl)ethylamino]-1H- | | 3H). | |
| | quinolin-2-one | | | |
| Example 233 Method 6 Mixture of Stereo- isomers | 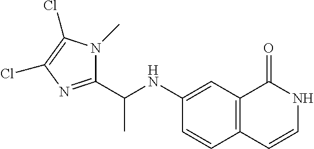 [SMILES: CC(Nc1ccc2cc[nH]c(=O)c2c1)c1nc(Cl)c(Cl)n1C] | White solid, yield: 34.7% | ¹H NMR (400 MHz, DMSO-d₆) δ 10.94 (s, J = 5.0 Hz, 1H), 7.39 (d, J = 8.6 Hz, 1H), 7.27 (d, J = 2.1 Hz, 1H), 7.09 (dd, J = 8.6, 2.4 Hz, 1H), 6.90-6.78 (m, 1H), 6.49 (d, J = 8.2 Hz, 1H), 6.36 (d, J = 7.0 Hz, 1H), 4.94- | 336.8 |
| | 7-((1-(4,5-dichloro- | | 4.86 (m, 1H), 3.60 (s, | |
| | 1-methyl-1H- | | 3H), 1.48 (d, J = 6.6 | |
| | imidazol-2- | | Hz, 3H). | |
| | yl)ethyl)amino)iso- | | | |
| | quinolin-1(2H)-one | | | |
| Eaxmple 234 Method 6 single unknown stereo- isomer | 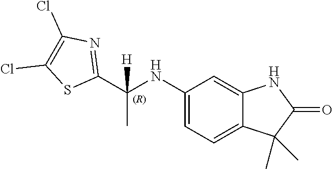 | White solid, yield: 32.4% | ¹H NMR (400 MHz, DMSO-d₆) δ 10.05 (s, 1H), 6.93 (d, J = 8.0 Hz, 1H), 6.50 (d, J = 5.2 Hz, 1H), 6.25-6.09 (m, 2H), 4.70-4.54 (m, 1H), 1.50 (d, J = 6.6 Hz, 3H), 1.14 (s, 6H). | 356.26 CHIRAL Cellulose SB, 2 cm × 25 cm, 5 um, Mobile Phase A: Hex, Mobile Phase B: EtOH |
| | 6-[[(1R)-1-(4,5- | | | Flow |
| | dichlorothiazol-2- | | | Rate: 20 mL/ |
| | yl)ethyl]amino]-3,3- | | | min |
| | dimethyl-indolin-2- | | | 220 nm, 25° C. |
| | one | | | Rt: 8.22 min. |
| Example 234A Method 6 single unknown stereo- isomer | 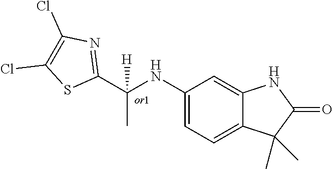 [SMILES: C[C@H](Nc1ccc2c(c1)NC(=O)C2(C)C)c1nc(Cl)c(Cl)s1] | White solid, yield: 33.8% | ¹H NMR (400 MHz, DMSO-d₆) δ 10.06 (s, 1H), 6.94 (d, J = 8.0 Hz, 1H), 6.50 (d, J = 5.3 Hz, 1H), 6.16 (d, J = 6.1 Hz, 2H), 4.76- 4.54 (m, 1H), 1.51 (d, J = 6.8 Hz, 3H), 1.15 (s, 6H). | 356.26 SHIRAL Cellulose SB, 2 cm × 25 cm, 5 um, Mobile Phase A: Hex, Mobile Phase B: EtOH |
| | 6-[[(1S)-1-(4,5- | | | Flow |
| | dichlorothiazol-2- | | | Rate: 20 mL/ |
| | yl)ethyl]amino]-3,3- | | | min |
| | dimethyl-indolin-2- | | | 220 nm, 25° C. |
| | one | | | Rt: 7.13 min. |
| Example 236 Method 6 Mixture of Stereo- isomers | 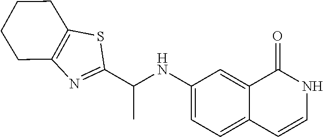 [SMILES: CC(Nc1ccc2cc[nH]c(=O)c2c1)c1nc2c(s1)CCCC2] | White solid, yield: 12.1% | ¹H NMR (400 MHz, CDCl₃) δ 10.03 (s, 1H), 7.54 (d, J = 1.2 Hz, 1H), 7.39 (d, J = 8.4 Hz, 1H), 7.06 (dd, J = 8.8, 2.8 Hz, 1H), 6.90 (s, 1H), 6.45 (d, J = 7.2 Hz, 1H), 4.97-5.02 (m, 1H), 2.79 (s, 2H), 2.69 | 326.0 |
| | 7-((1-(4,5,6,7- | | (s, 2H), 1.93-1.77 (m, | |
| | tetrahydrobenzo[d]thi- | | 4H), 1.71 (d, J = 6.8 | |
| | azol-2- | | Hz, 3H). | |
| | yl)ethyl)amino)iso- | | | |
| | quinolin-1(2H)-one | | | |
| Example 245 Method 6 Mixture of Stereo- isomers | 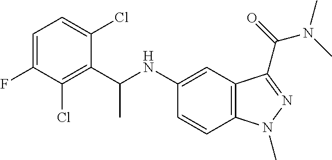 [SMILES: CC(Nc1ccc2c(c1)c(C(=O)N(C)C)nn2C)c1c(Cl)ccc(F)c1Cl] | white solid, yield: 24.8% | ¹H NMR (400 MHz, DMSO-d₆) δ 7.57- 7.26 (m, 1H), 7.40 (d, J = 9.0 Hz, 1H), 7.31 (t, J = 8.6 Hz, 1H), 6.96 (dd, J = 9.0, 2.2 Hz, 1H), 6.69 (d, J = 2.1 Hz, 1H), 6.29 (d, J = 8.3 Hz, 1H), 5.26 (p, J = 7.1 Hz, 1H), 3.95 (s, | 219.6 |
| | 5-((1-(2,6-dichloro- | | 3H), 3.16 (s, 3H), 3.00 | |
| | 3- | | (s, 3H), 1.61 (d, J = 7.0 | |
| | fluorophenyl)ethyl)a- | | Hz, 3H). | |
| | mino)-N,N1- | | | |
| | trimethyl-1H- | | | |
| | indazole-3- | | | |
| | carboxamide | | | |
| Example 246 Method 6 Mixture of Stereo- isomers | 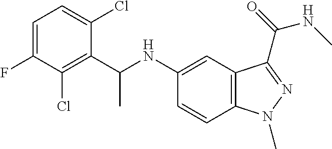 [SMILES: CNC(=O)c1nn(C)c2ccc(NC(C)c3c(Cl)ccc(F)c3Cl)cc12] | White solid, yield: 3.10% | ¹H NMR (400 MHz, DMSO-d₆) δ 7.95 (q, J = 4.7 Hz, 1H), 7.57- 7.41 (m, 1H), 7.40 (d, J = 9.0 Hz, 1H), 7.30 (t, J = 8.6 Hz, 1H), 7.01- 6.91 (m, 2H), 6.30 (d, J = 8.3 Hz, 1H), 5.28 (p, J = 7.2 Hz, 1H), 3.96 (s, 3H), 2.74 (d, J = 4.6 | 395.2 |
| | 5-((1-(2,6-dichloro- | | Hz, 3H), 1.61 (d, J = | |
| | 3- | | 6.9 Hz, 3H). | |
| | fluorophenyl)ethyl)a- | | | |
| | mino)-N,1-dimethyl- | | | |
| | 1H-indazole-3- | | | |
| | carboxamide | | | |
| Example 249 Method 6 Mixture of Stereo- isomers | 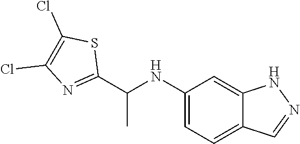 [SMILES: CC(Nc1ccc2cn[nH]c2c1)c1nc(Cl)c(Cl)s1] | Off- white solid, yield: 20.0% | ¹H NMR (400 MHz, DMSO-d₆) δ 7.78 (s, 1H), 7.46 (d, J = 8.7 Hz, 1H), 6.76 (s, 1H), 6.64 (dd, J = 8.6, 1.9 Hz, 1H), 6.35 (s, 1H), 4.76 (q, J = 6.6 Hz, 1H), 1.56 (d, J = 6.7 Hz, 3H). | 313 |
| | N-(1-(4,5- | | | |
| | dichlorothiazol-2- | | | |
| | yl)ethyl)-1H-indazol- | | | |
| | 6-amine | | | |
| Example 257 Method 6 Mixture of Stereo- isomers | 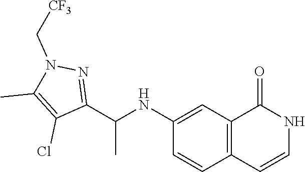 [SMILES: Cc1c(Cl)c(C(C)Nc2ccc3cc[nH]c(=O)c3c2)nn1CC(F)(F)F] | white solid, yield: 9.5% | ¹H NMR (400 MHz, DMSO-d₆) δ 10.84 (d, J = 5.6 Hz, 1H), 7.31 (d, J = 8.6 Hz, 1H), 7.24 (d, J = 2.5 Hz, 1H), 7.06 (dd, J = 8.6, 2.6 Hz, 1H), 6.80 (dd, J = 7.0, 5.7 Hz, 1H), 6.38 (d, J = 7.6 Hz, 1H), 6.32 (d, J = 7.0 Hz, 1H), 5.12-4.99 (m, 2H), | 385.1 |
| | 7-((1-(4-chloro-5- | | 4.66 (p, J = 6.9 Hz, | |
| | methyl-1-(2,2,2- | | 1H), 2.23 (s, 3H), 1.50 | |
| | trifluoroethyl)-1H- | | (d, J = 6.8 Hz, 3H). | |
| | pyrazol-3- | | | |
| | yl)ethyl)amino)iso- | | | |
| | quinolin-1(2H)-one | | | |
| Example 258 Method 6 Mixture of Stereo- isomers | 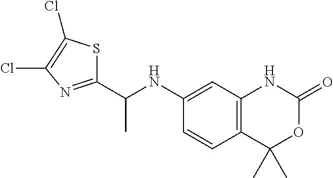 [SMILES: CC(Nc1ccc2c(c1)NC(=O)OC2(C)C)c1nc(Cl)c(Cl)s1] | white solid, yield: 15.2% | ¹H NMR (400 MHz, DMSO-d₆) δ 9.93 (s, 1H), 6.96 (d, J = 8.3 Hz, 1H), 6.70 (d, J = 5.4 Hz, 1H), 6.25 (dd, J = 8.4, 2.3 Hz, 1H), 6.13 (d, J = 2.2 Hz, 1H), 4.62 (p, J = 6.6 Hz, 1H), 1.57-1.38 (m, 9H). | 372.2 |
| | 7-((1-(4,5- | | | |
| | dichlorothiazol-2- | | | |
| | yl)ethyl)amino)-4,4- | | | |
| | dimethyl-1,4- | | | |
| | dihydro-2H- | | | |
| | benzo[d][1,3]oxazin- | | | |
| | 2-one | | | |
| Example 259 Method 6 Mixture of Stereo- isomers | 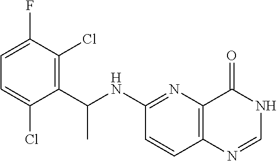 [SMILES: CC(Nc1ccc2nc[nH]c(=O)c2n1)c1c(Cl)ccc(F)c1Cl] | White solid, yield: 21.3% | ¹H NMR (400 MHz, DMSO-d₆) δ 7.77 (s, 1H), 7.65 (d, J = 9.0 Hz, 1H), 7.37 (dd, J = 8.8, 4.9 Hz, 1H), 7.25 (t, J = 8.6 Hz, 1H), 7.04 (d, J = 9.0 Hz, 1H), 5.77 (q, J = 7.3 Hz, 1H), 1.56 (d, J = 7.3 Hz, 3H). | 353.1 |
| | 6-((1-(2,6-dichloro- | | | |
| | 3- | | | |
| | fluorophenyl)ethyl)a- | | | |
| | mino)pyrido[3,2- | | | |
| | d]pyrimidin-4(3H)- | | | |
| | one | | | |
| Example 260 Method 6 Mixture of Stereo- isomers | 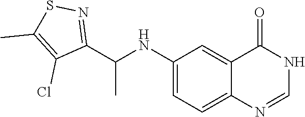 [SMILES: Cc1snc(C(C)Nc2ccc3nc[nH]c(=O)c3c2)c1Cl] | White solid, yield: 55% | ¹H NMR (400 MHz, DMSO-d₆) δ 11.86 (s, 1H), 7.76 (d, J = 3.4 Hz, 1H), 7.39 (d, J = 8.8 Hz, 1H), 7.16 (dd, J = 8.8, 2.8 Hz, 1H), 7.02 (d, J = 2.8 Hz, 1H), 6.75 (d, J = 7.9 Hz, | 321.3 |
| | 6-((1-(4-chloro-5- | | 1H), 4.87-4.82 (m, | |
| | methylisothiazol-3- | | 1H), 2.47 (s, 3H), 1.52 | |
| | yl)ethyl)amino)quina- | | (d, J = 6.7 Hz, 3H). | |
| | zolin-4(3H)-one | | | |
| Example 261 Method 6 Mixture of Stereo- isomers | 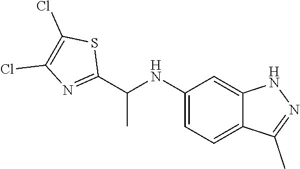 [SMILES: Cc1n[nH]c2cc(NC(C)c3nc(Cl)c(Cl)s3)ccc12] | white solid, yield: 3.1% | ¹H NMR (400 MHz, DMSO-d₆) δ 11.95 (s, 1H), 7.39 (d, J = 8.6 Hz, 1H), 6.71 (d, J = 5.4 Hz, 1H), 6.59 (dd, J = 8.7, 1.9 Hz, 1H), 6.28 (d, J = 1.8 Hz, 1H), 4.75 (q, J = 6.9, 5.5 Hz, 1H), 2.34 (s, 3H), 1.56 (d, J = 6.7 Hz, 3H). | 327 |
| | N-(1-(4,5- | | | |
| | dichlorothiazol-2- | | | |
| | yl)ethyl)-3-methyl- | | | |
| | 1H-indazol-6-amine | | | |
| Example 262 Method 6 Mixture of Stereo- isomers | 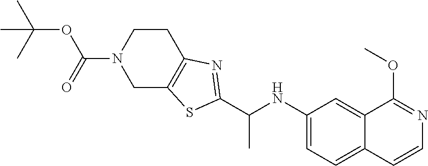 [SMILES: COc1nccc2ccc(NC(C)c3nc4c(s3)CN(C(=O)OC(C)(C)C)CC4)cc12] | white solid, yield: 15.9% | ¹H NMR (400 MHz, DMSO-d₆) δ 7.67 (d, J = 5.7 Hz, 1H), 7.62 (d, J = 8.9 Hz, 1H), 7.24 (dd, J = 8.8, 2.4 Hz, 1H), 7.14 (d, J = 5.7 Hz, 1H), 7.00 (d, J = 2.4 Hz, 1H), 6.96 (d, J = 6.5 Hz, 1H), 4.86 (p, J = 6.6 Hz, 1H), 4.48 (s, 2H), 3.95 (s, 3H), 3.63 | 441 |
| | tert-butyl 2-(1-((1- | | (s, 2H), 2.79-2.71 (m, | |
| | methoxyisoquinolin- | | 2H), 1.57 (d, J = 6.7 | |
| | 7-yl)amino)ethyl)- | | Hz, 3H), 1.39 (s, 9H). | |
| | 6,7- | | | |
| | dihydrothiazolo[5,4- | | | |
| | c]pyridine-5(4H)- | | | |
| | carboxylate | | | |
| Example 263 Method 6 Mixture of Stereo- isomers | 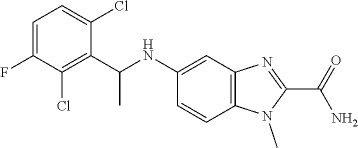 [SMILES: CC(Nc1ccc2c(c1)nc(C(N)=O)n2C)c1c(Cl)ccc(F)c1Cl] | yellow solid, yield: 17.3% | ¹H NMR (400 MHz, CDCl₃) δ 7.45 (s, 1H), 7.19 (d, J = 9.2 Hz, 1H), 6.97-6.89 (m, 1H), 6.85-6.78 (m, 2H), 5.47 (s, 2H), 4.78 (s, 1H), 4.10 (s, 3H), 2.01 (s, 1H), 1.69 (d, J = 7.2 Hz, 3H). | 382.9 |
| | 5-((1-(2,6-dichloro- | | | |
| | 3- | | | |
| | fluorophenyl)ethyl)a- | | | |
| | mino)-1-methyl-1H- | | | |
| | benzo[d]imidazole-2- | | | |
| | carboxamide | | | |
| Example 266 Method 6 Mixture of Stereo- isomers | 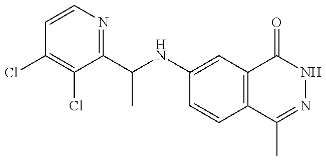 [SMILES: Cc1n[nH]c(=O)c2cc(NC(C)c3nccc(Cl)c3Cl)ccc12] | white solid, yield: 24.8% | ¹H NMR (400 MHz, DMSO-d₆) δ 11.98 (s, 1H), 8.46 (d, J = 5.0 Hz, 1H), 7.66 (d, J = 5.0 Hz, 1H), 7.60 (d, J = 8.8 Hz, 1H), 7.32 (d, J = 8.2 Hz, 1H), 7.17 (d, J = 8.8 Hz, 1H), 7.11 (s, 1H), 5.21 (dd, J = 14.2, 6.9 Hz, 1H), | 349.0 |
| | 7-((1-(3,4- | | 2.36 (s, 3H), 1.52 (d, J = | |
| | dichloropyridin-2- | | 6.6 Hz, 3H). | |
| | yl)ethyl)amino)-4- | | | |
| | methylphthalazin- | | | |
| | 1(2H)-one | | | |
| Example 267 Method 6 Mixture of Stereo- isomers | 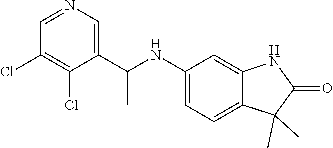 [SMILES: CC(Nc1ccc2c(c1)NC(=O)C2(C)C)c1cncc(Cl)c1Cl] | yellow solid, yield: 28.1% | ¹H NMR (400 MHz, DMSO-d₆) δ 10.02 (s, 1H), 8.64 (s, 1H), 8.53 (s, 1H), 6.88 (d, J = 8.0 Hz, 1H), 6.50 (d, J = 6.8 Hz, 1H), 6.02 (s, 1H), 5.97 (dd, J = 8.0, 1.9 Hz, 1H), 4.81-4.74 (m, 1H), 1.47 (d, J = | 349.9 |
| | 6-((1-(4,5- | | 6.8 Hz, 3H), 1.12 (s, | |
| | dichloropyridin-3- | | 3H), 1.11 (s, 3H) | |
| | yl)ethyl)amino)-3,3- | | | |
| | dimethylindolin-2- | | | |
| | one | | | |
| Example 271 Method 6 Mixture of Stereo- isomers | 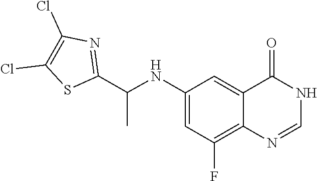 [SMILES: CC(Nc1cc(F)c2nc[nH]c(=O)c2c1)c1nc(Cl)c(Cl)s1] | white solid, yield: 65.6% | ¹H NMR (400 MHz, DMSO-d₆) δ 12.13 (s, 1H), 7.85 (s, 1H), 7.23 (d, J = 6.3 Hz, 1H), 7.08 (dd, J = 12.8, 2.5 Hz, 1H), 6.95 (d, J = 2.5 Hz, 1H), 4.93 (t, J = 6.7 Hz, 1H), 1.56 (d, J = 6.7 Hz, 3H). | 359.2 |
| | 6-((1-(4,5- | | | |
| | dichlorothiazol-2- | | | |
| | yl)ethyl)amino)-8- | | | |
| | fluoroquinazolin- | | | |
| | 4(3H)-one | | | |
| Example 275 Method 6 Mixture of Stereo- isomers | 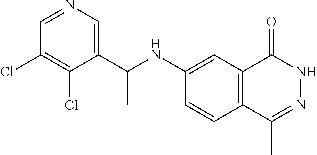 [SMILES: Cc1n[nH]c(=O)c2cc(NC(C)c3cncc(Cl)c3Cl)ccc12] | yellow solid, yield: 11.5% | ¹H NMR (400 MHz, DMSO-d₆) δ 12.02 (s, 1H), 8.67 (s, 1H), 8.53 (s, 1H), 7.65 (d, J = 8.8 Hz, 1H), 7.49 (d, J = 7.2 Hz, 1H), 7.12 (dd, J = 8.8, 2.2 Hz, 1H), 7.02 (s, 1H), 5.00 (t, J = 6.8 Hz, 1H), 2.36 (s, 3H), 1.56 (d, J = 6.8 Hz, | 349.1 |
| | 7-((1-(4,5- | | 3H). | |
| | dichloropyridin-3- | | | |
| | yl)ethyl)amino)-4- | | | |
| | methylphthalazin- | | | |
| | 1(2H)-one | | | |
| Example 277 Method 6 Mixture of Stereo- isomers | 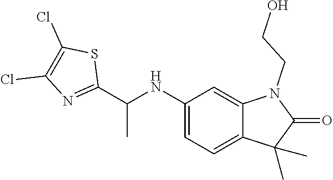 [SMILES: CC(Nc1ccc2c(c1)N(CCO)C(=O)C2(C)C)c1nc(Cl)c(Cl)s1] | white solid, yield: 36.4% | ¹H NMR (400 MHz, DMSO-d₆) δ 7.01 (d, J = 8.0 Hz, 1H), 6.53 (d, J = 6.0 Hz, 1H), 6.42 (d, J = 2.0 Hz, 1H), 6.19 (dd, J = 8.0, 2.0 Hz, 1H), 4.82 (t, J = 5.6 Hz, 1H), 4.76 (p, J = 6.4 Hz, 1H), 3.63 (t, J = 6.0 Hz, 2H), 3.53 (q, J = 5.6 Hz, 2H), 1.52 (d, | 400.0/402.0 |
| | 6-((1-(4,5- | | J = 6.8 Hz, 3H), 1.18 (s, | |
| | dichlorothiazol-2- | | 6H). | |
| | yl)ethyl)amino)-1-(2- | | | |
| | hydroxyethyl)-3,3- | | | |
| | dimethylindolin-2- | | | |
| | one | | | |
| Example 278 Method 6 Mixture of Stereo- isomers | 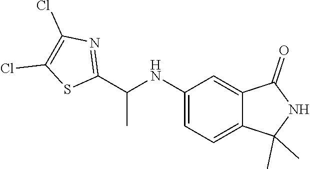 [SMILES: CC(Nc1ccc2c(c1)C(=O)NC2(C)C)c1nc(Cl)c(Cl)s1] | white solid, yield: 35.3% | ¹H NMR (400 MHz, DMSO-d₆) δ 8.47 (s, 1H), 7.31 (d, J = 8.2 Hz, 1H), 6.87 (dd, J = 8.3, 2.3 Hz, 1H), 6.76 (d, J = 5.6 Hz, 1H), 6.70 (d, J = 2.2 Hz, 1H), 4.84-4.70 (m, 1H), 1.54 (d, J = 6.7 Hz, 3H), 1.36 (d, J = 1.6 Hz, 6H). | 356.26 |
| | 6-[1-(4,5- | | | |
| | dichlorothiazol-2- | | | |
| | yl)ethylamino]-3,3- | | | |
| | dimethyl-isoindolin- | | | |
| | 1-one | | | |
| Example 282 Method 6 Mixture of Stereo- isomers | 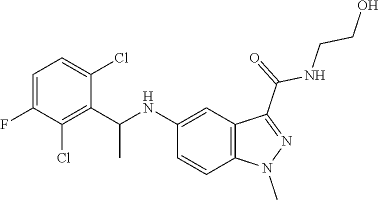 [SMILES: CC(Nc1ccc2c(c1)c(C(=O)NCCO)nn2C)c1c(Cl)ccc(F)c1Cl] | White solid, yield: 8.09% | ¹H NMR (400 MHz, DMSO-d₆) δ 7.81 (t, J = 5.8 Hz, 1H), 7.41 (d, J = 9.0 Hz, 1H), 7.30 (t, J = 8.6 Hz, 1H), 7.01- 6.91 (m, 2H), 6.32 (d, J = 8.2 Hz, 1H), 5.28 (p, J = 7.1 Hz, 1H), 4.75 (t, J = 5.4 Hz, 1H), 3.96 (s, 3H), 3.49 (q, J = 5.9 Hz, 2H), 3.37-3.27 (m, 2H), 1.60 (d, J = | 425.2 |
| | 5-((1-(2,6-dichloro- | | 7.0 Hz, 3H). | |
| | 3- | | | |
| | fluorophenyl)ethyl)a- | | | |
| | mino)-N-(2- | | | |
| | hydroxyethyl)-1- | | | |
| | methyl-1H-indazol- | | | |
| | 3-carboxamide | | | |
| Example 288 Method 6 Mixture of Stereo- isomer | 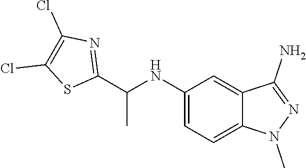 [SMILES: CC(Nc1ccc2c(c1)c(N)nn2C)c1nc(Cl)c(Cl)s1] | gray solid, yield: 19.3% | ¹H NMR (400 MHz, Chloroform-d) δ 7.14 (d, J = 9.0 Hz, 1H), 7.06 (d, J = 9.0 Hz, 1H), 6.58 (s, 1H), 4.70 (q, J = 6.6 Hz, 1H), 3.74 (s, 3H), 1.70 (d, J = 6.5 Hz, 3H). | 342 |
| | N5-(1-(4,5- | | | |
| | dichlorothiazol-2- | | | |
| | yl)ethyl)-1-methyl- | | | |
| | 1H-indazole-3,5- | | | |
| | diamine | | | |
| Example 302 Method 6 single unknown stereo- isomer | 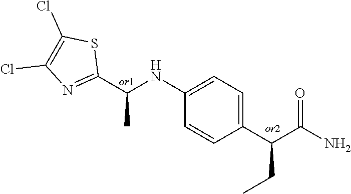 [SMILES: CC[C@H](C(N)=O)c1ccc(N[C@@H](C)c2nc(Cl)c(Cl)s2)cc1] | white solid, yield: 6.0% | NMR (400 MHz, DMSO-d₆) δ 7.26 (d, J = 2.0 Hz, 1H), 7.01- 6.98 (m, 2H), 6.65 (s, 1H), 6.50-6.47 (m, 2H), 6.37 (d, J = 5.6 Hz, 1H), 4.62 (m, 1H), 3.09-3.05 (m, 1H), 1.88-1.73 (m, 1H), 1.49-1.44 (m, 4H), 0.73 (t, J = 7.4 Hz, 3H). | 357.9 Note: SFC Thar prep 80 CHIRALPAK WHELK- 01, 250 mm × 21.1 mm, 5 μm, Modifier: 40% EtOH (NH₄OH |
| | rel-(R)-2-(4-(((R)-1- | | | 0.2%) |
| | (4,5-dichlorothiazol- | | | Rt = 3.64 min |
| | 2- | | | |
| | yl)ethyl)amino)phenyl) | | | |
| | butanamide | | | |
| Example 302A Method 6 single unknown stereo- isomer | 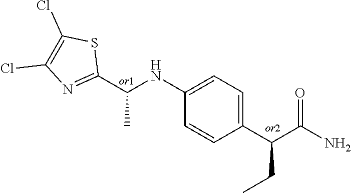 [SMILES: CC[C@H](C(N)=O)c1ccc(N[C@H](C)c2nc(Cl)c(Cl)s2)cc1] | white solid, yield: 83.9% | ¹H NMR (400 MHz, DMSO-d₆) δ 7.26 (d, J = 2.0 Hz, 1H), 7.01- 6.98 (m, 2H), 6.65 (s, 1H), 6.50-6.47 (m, 2H), 6.37 (d, J = 5.6 Hz, 1H), 4.62 (m, 1H), 3.09-3.05 (m, 1H), 1.88-1.73 (m, 1H), 1.49-1.44 (m, 4H), 0.73 (t, J = 7.4 Hz, 3H). | 357.9 Note: SFC Thar prep 80 CHIRALPAK WHELK- 01, 250 mm × 21.1 mm, 5 μm, Modifier: 40% EtOH (NH₄OH |
| | rel-(R)-2-(4-(((S)-1- | | | 0.2%) |
| | (4,5-dichlorothiazol- | | | Rt = 1.94 min |
| | 2- | | | |
| | yl)ethyl)amino)phenyl) | | | |
| | butanamide | | | |
| Example 302B Method 6 single unknown stereo- isomer | 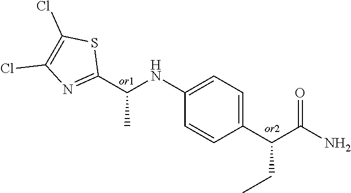 [SMILES: CC[C@@H](C(N)=O)c1ccc(N[C@H](C)c2nc(Cl)c(Cl)s2)cc1] | white solid, yield: 6.16% | ¹H NMR (400 MHz, DMSO-d₆) δ 7.26 (d, J = 2.0 Hz, 1H), 7.01- 6.98 (m, 2H), 6.65 (s, 1H), 6.50-6.47 (m, 2H), 6.37 (d, J = 5.6 Hz, 1H), 4.62 (m, 1H), 3.09-3.05 (m, 1H), 1.88-1.73 (m, 1H), 1.49-1.44 (m, 4H), 0.73 (t, J = 7.4 Hz, 3H). | 357.9 Note: SFC Thar prep 80 CHIRALPAK WHELK- 01, 250 mm × 21.1 mm, 5 μm, Modifier: 40% EtOH (NH₄OH |
| | rel-(R)-2-(4-(((R)-1- | | | 0.2%) |
| | (4,5-dichlorothiazol- | | | Rt = 3.23 min |
| | 2- | | | |
| | yl)ethyl)amino)phenyl) | | | |
| | butanamide | | | |
| Example 302C Method 6 single unknown stereo- isomer | 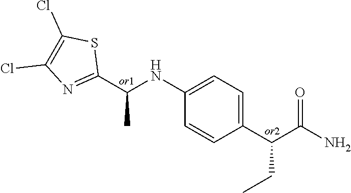 [SMILES: CC[C@@H](C(N)=O)c1ccc(N[C@@H](C)c2nc(Cl)c(Cl)s2)cc1] | white solid, yield: 9.30% | ¹H NMR (400 MHz, DMSO-d₆) δ 7.26 (d, J = 2.0 Hz, 1H), 7.01- 6.98 (m, 2H), 6.65 (s, 1H), 6.50-6.47 (m, 2H), 6.37 (d, J = 5.6 Hz, 1H), 4.62 (m, 1H), 3.09-3.05 (m, 1H), 1.88-1.73 (m, 1H), 1.49-1.44 (m, 4H), 0.73 (t, J = 7.4 Hz, 3H). | 357.9 Note: SFC Thar prep 80 CHIRALPAK WHELK- 01, 250 mm × 21.1 mm, 5 μm, Modifier: 40% EtOH (NH₄OH |
| | rel-(R)-2-(4-(((S)-1- | | | 0.2%) |
| | (4,5-dichlorothiazol- | | | Rt = 5.34 min |
| | 2- | | | |
| | yl)ethyl)amino)phenyl) | | | |
| | butanamide | | | |
| Example 310 Method 6 Mixture of Stereo- isomers | 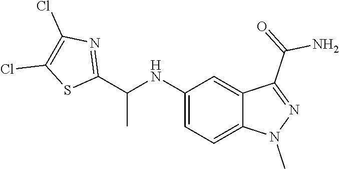 [SMILES: CC(Nc1ccc2c(c1)c(C(N)=O)nn2C)c1nc(Cl)c(Cl)s1] | white solid, yield: 56.8% | ¹H NMR (400 MHz, DMSO-d₆) δ 7.51 (d, J = 9.0 Hz, 1H), 7.45 (s, 1H), 7.17-7.11 (m, 2H), 7.00 (dd, J = 9.0, 2.2 Hz, 1H), 6.57 (d, J = 5.6 Hz, 1H), 4.78- 4.70 (m, 1H), 4.01 (s, 3H), 1.56 (d, J = 6.7 Hz, 3H). | 370.1 |
| | 5-((1-(4,5- | | | |
| | dichlorothiazol-2- | | | |
| | yl)ethyl)amino)-1- | | | |
| | methyl-1H-indazole- | | | |
| | 3-carboxamide | | | |
| Example 311 Method 6 Mixture of Stereo- isomers | 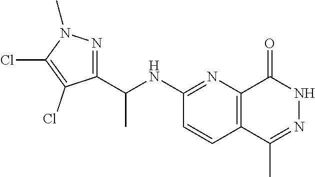 [SMILES: Cc1n[nH]c(=O)c2nc(NC(C)c3nn(C)c(Cl)c3Cl)ccc12] | White solid, yield: 56% | ¹H NMR (400 MHz, DMSO-d₆) δ 12.32 (s, 1H), 8.01 (s, 1H), 7.89 (d, J = 7.5 Hz, 1H), 7.05 (d, J = 9.0 Hz, 1H), 5.51 (s, 1H), 3.81 (s, 3H), 2.37 (s, 3H), 1.50 (d, J = 6.8 Hz, 3H). | 353.1 |
| | 2-((1-(4,5-dichloro- | | | |
| | 1-methyl-1H- | | | |
| | pyrazol-3- | | | |
| | yl)ethyl)amino)-5- | | | |
| | methylpyrido[2,3- | | | |
| | d]pyridazin-8(7H)- | | | |
| | one | | | |
| Example 314 Method 6 Mixture of Stereo- isomers | 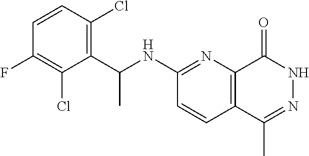 [SMILES: Cc1n[nH]c(=O)c2nc(NC(C)c3c(Cl)ccc(F)c3Cl)ccc12] | White solid, yield: 21% | ¹H NMR (400 MHz, DMSO-d₆) δ 12.10 (s, 1H), 8.21 (s, 1H), 7.86 (d, J = 9.0 Hz, 1H), 7.41 (s, 1H), 7.30 (t, J = 8.7 Hz, 1H), 7.04 (d, J = 22.5 Hz, 1H), 5.85 (s, 1H), 2.33 (s, 3H), 1.61 (d, J = 7.2 Hz, 3H). | 367.6 |
| | 2-((1-(2,6-dichloro- | | | |
| | 3- | | | |
| | fluorophenyl)ethyl)a- | | | |
| | mino)-5- | | | |
| | methylpyrido[2,3- | | | |
| | d]pyridazin-8(7H)- | | | |
| | one | | | |
| Example 315 Method 6 Mixture of Stereo- isomers | 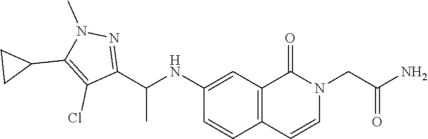 [SMILES: CC(Nc1ccc2ccn(CC(N)=O)c(=O)c2c1)c1nn(C)c(C2CC2)c1Cl] | yellow solid, yield: 15.5% | ¹H NMR (400 MHz, DMSO-d₆) δ 7.54 (s, 1H), 7.33 (d, J = 8.4 Hz, 1H), 7.22 (d, J = 2.4 Hz, 1H), 7.12 (s, 1H), 7.08 (dd, J = 8.8 Hz, 2.4 Hz, 1H), 7.00 (d, J = 7.2 Hz, 1H), 6.37-6.34 (m, 2H), | 399.9 |
| | 2-(7-((1-(4-chloro-5- | | 4.64-4.56 (m, 1H), | |
| | cyclopropyl-1- | | 4.53-4.43 (m, 2H), 3.76 | |
| | methyl-1H-pyrazol- | | (s, 3H), 1.77-1.69 (m, | |
| | 3-yl)ethyl)amino)-1- | | 1H), 1.46 (d, J = 6.8 | |
| | oxoisoquinolin- | | Hz, 3H), 0.98-0.92 (m, | |
| | 2(1H)-yl)acetamide | | 2H), 0.82-0.77 (m, 2H). | |
| Example 316 Method 6 Mixture of Stereo- isomers | 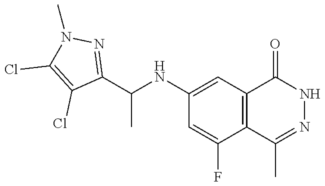 [SMILES: Cc1n[nH]c(=O)c2cc(NC(C)c3nn(C)c(Cl)c3Cl)cc(F)c12] | white solid, yield: 47.3% | ¹H NMR (400 MHz, CDCl₃) δ 7.36 (d, J = 2.4 Hz, 1H), 6.76 (dd, J = 14.0, 2.4 Hz, 1H), 4.81-4.83 (m, 1H), 3.81 (s, 3H), 2.58 (d, J = 6.4 Hz, 3H), 1.60 (d, J = 6.8 Hz, 3H). | 369.9 |
| | 7-((1-(4,5-dichloro- | | | |
| | 1-methyl-1H- | | | |
| | pyrazol-3- | | | |
| | yl)ethyl)amino)-5- | | | |
| | fluoro-4- | | | |
| | methylphthalazin- | | | |
| | 1(2H)-one | | | |
| Example 318 Method 6 Mixture of Stereo- isomers | 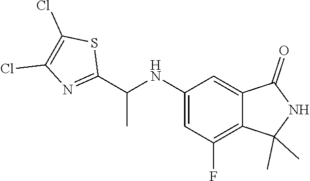 [SMILES: CC(Nc1cc(F)c2c(c1)C(=O)NC2(C)C)c1nc(Cl)c(Cl)s1] | white solid, yield: 10.9% | ¹H NMR (400 MHz, DMSO-d₆) δ 8.69 (s, 1H), 7.04 (d, J = 6.4 Hz, 1H), 6.63 (d, J = 11.2 Hz, 2H), 4.85 (p, J = 6.8 Hz, 1H), 1.53 (d, J = 6.8 Hz, 3H), 1.43 (d, J = 1.6 Hz, 6H). | 374.1/376.1 |
| | 6-((1-(4,5- | | | |
| | dichlorothiazol-2- | | | |
| | yl)ethyl)amino)-4- | | | |
| | fluoro-3,3- | | | |
| | dimethylisoindolin-1- | | | |
| | one | | | |
| Example 321 Method 6 Mixture of Stereo- isomers | 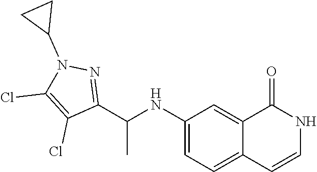 [SMILES: CC(Nc1ccc2cc[nH]c(=O)c2c1)c1nn(C2CC2)c(Cl)c1Cl] | Off white solid, yield: 27% | ¹H NMR (400 MHz, CDCl₃) δ 9.13 (s, 1H), 7.57 (d, J = 2.4 Hz, 1H), 7.36 (d, J = 8.8 Hz, 1H), 7.04 (dd, J = 8.4, 2.4 Hz, 1H), 6.86- 6.83 (m, 1H), 6.41 (d, J = 7.2 Hz, 1H), 4.85 (t, J = 7.2 Hz, 1H), 4.59 (s, 1H), 3.43-3.37 (m, 1H), 1.25-1.11 (m, 3H), | 363.0 |
| | 7-((1-(4,5-dichloro- | | 1.07-1.05 (m, 2H). | |
| | 1-cyclopropyl-1H- | | | |
| | pyrazol-3- | | | |
| | yl)ethyl)amino)iso- | | | |
| | quinolin-1(2H)-one | | | |
| Example 322 Method 6 Mixture of Stereo- isomer | 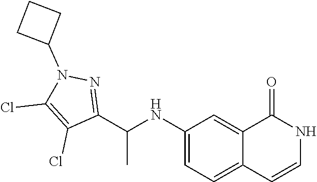 [SMILES: CC(Nc1ccc2cc[nH]c(=O)c2c1)c1nn(C2CCC2)c(Cl)c1Cl] | yellow solid, yield: 15.5% | ¹H NMR (400 MHz, DMSO-d₆) δ 10.86 (d, J = 5.6 Hz, 1H), 7.34 (d, J = 8.6 Hz, 1H), 7.27 (d, J = 2.4 Hz, 1H), 7.09 (dd, J = 8.6, 2.5 Hz, 1H), 6.81 (dd, J = 6.9, 5.8 Hz, 1H), 6.47 (d, J = 7.1 Hz, 1H), 6.33 (d, J = 6.8 Hz, 1H), 4.93-4.84 (m, 1H), 4.72-4.63 (m, 1H), | 376.9 |
| | 7-((1-(4,5-dichloro- | | 2.62-2.52 (m, 2H), | |
| | 1-cyclobutyl-1H- | | 2.37-2.27 (m, 2H), | |
| | pyrazol-3- | | 1.86-1.75 (m, 2H), 1.55 | |
| | yl)ethyl)amino)iso- | | (d, J = 6.8 Hz, 3H). | |
| | quinolin-1(2H)-one | | | |
| Example 324 Method 6 Mixture of Stereo- isomers | 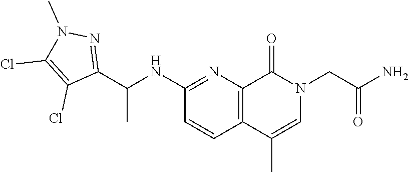 [SMILES: Cc1cn(CC(N)=O)c(=O)c2nc(NC(C)c3nn(C)c(Cl)c3Cl)ccc12] | white solid, yield: 39.8% | ¹H NMR (400 MHz, CDCl₃) δ 7.75 (d, J = 9.2 Hz, 1H), 7.18 (s, 1H), 6.91 (d, J = 9.2 Hz, 1H), 6.86 (s, 1H), 6.18 (s, 1H), 5.60 (s, 1H), 5.17-5.07 (m, 1H), 4.66 (s, 2H), 3.79 (s, 3H), 2.20 (s, 3H), 1.63 (d, J = 6.8 Hz, 3H). | 408.9 |
| | 2-(2-((1-(4,5- | | | |
| | dichloro-1-methyl- | | | |
| | 1H-pyrazol-3- | | | |
| | yl)ethyl)amino)-5- | | | |
| | methyl-8-oxo-1,7- | | | |
| | naphthyridin-7(8H)- | | | |
| | yl)acetamide | | | |
| Example 325 Method 6 Mixture of Stereo- isomers | 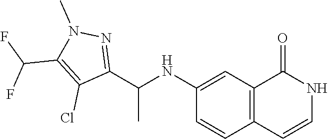 [SMILES: CC(Nc1ccc2cc[nH]c(=O)c2c1)c1nn(C)c(C(F)F)c1Cl] | yellow solid, yield: 15.5% | ¹H NMR (400 MHz, DMSO-d₆) δ 10.88 (d, J = 5.2 Hz, 1H), 7.39 (s, 0.25H), 7.35 (d, J = 8.4 Hz, 1H), 7.26 (s, 0.5H), 7.22 (d, J = 2.4 Hz, 1H), 7.13 (s, 0.25H), 7.07 (dd, J = 8.8, 2.4 Hz, 1H), 6.86-6.77 (m, | 342.9 |
| | 7-((1-(4-chloro-5- | | 1H), 6.46 (d, J = 7.6 | |
| | (difluoromethyl)-1- | | Hz, 1H), 6.34 (d, J = | |
| | methyl-1H-pyazol- | | 6.8 Hz, 1H), 4.77-4.63 | |
| | 3- | | (m, 1H), 3.89 (s, 3H), | |
| | yl)ethyl)amino)iso- | | 1.51 (d, J = 6.8 Hz, 3H) | |
| | quinolin-1(2H)-one | | | |
| Example 330 Method 6 Mixture of Stereo- isomers | 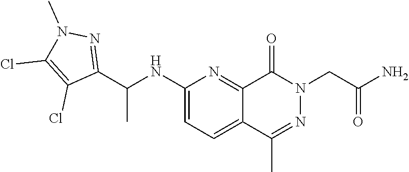 [SMILES: Cc1nn(CC(N)=O)c(=O)c2nc(NC(C)c3nn(C)c(Cl)c3Cl)ccc12] | White solid, yield: 23% | ¹H NMR (400 MHz, DMSO-d₆) δ 8.02 (d, J = 7.6 Hz, 1H), 7.89 (d, J = 9.0 Hz, 1H), 7.46 (s, 1H), 7.13 (s, 1H), 7.02 (d, J = 9.0 Hz, 1H), 5.52 (s, 1H), 4.58 (s, 2H), 3.80 (s, 3H), 2.38 (s, 3H), 1.49 (d, J = 7.0 Hz, 3H). | 410.1 |
| | 2-(2-((1-(4,5- | | | |
| | dichloro-1-methyl- | | | |
| | 1H-pyrazol-3- | | | |
| | yl)ethyl)amino)-5- | | | |
| | methyl-8- | | | |
| | oxopyrido[2,3- | | | |
| | d]pyridazin-7(8H)- | | | |
| | yl)acetamide | | | |
| Example 342 Method 6 Mixture of Stereo- isomers | 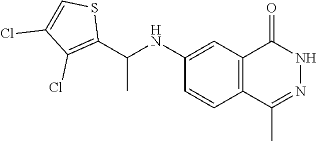 [SMILES: Cc1n[nH]c(=O)c2cc(NC(C)c3scc(Cl)c3Cl)ccc12] | yellow solid, yield: 14.8% | 12.04 (s, 1H), 7.68-7.65 (m, 2H), 7.44 (d, J = 5.6 Hz, 1H), 7.14-7.10 (m, 2H), 4.91 (t, J = 6.4 Hz, 1H), 2.38 (s, 3H), 1.56 (d, J = 6.8 Hz, 3H). | 354.0 |
| | 7-((1-(3,4- | | | |
| | dichlorothiophen-2- | | | |
| | yl)ethyl)amino)-4- | | | |
| | methylphthalazin- | | | |
| | 1(2H)-one | | | |
| Example 349 Method 6 Mixture of Stereo- isomers | 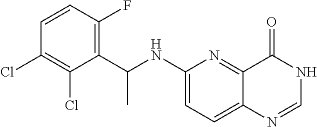 [SMILES: CC(Nc1ccc2nc[nH]c(=O)c2n1)c1c(F)ccc(Cl)c1Cl] | White solid, yield: 30.3% | ¹H NMR (400 MHz, DMSO-d₆) δ 12.11 (s, 1H), 7.82 (s, 1H), 7.73 (d, J = 7.6 Hz, 1H), 7.66 (d, J = 9.2 Hz, 1H), 7.55 (dd, J = 8.8, 5.2 Hz, 1H), 7.22 (dd, J = 10.4, 8.8 Hz, | 353.2 |
| | 6-((1-(2,3-dichloro- | | 1H), 7.04 (d, J = 9.2 | |
| | 6- | | Hz, 1H), 5.85-5.66 | |
| | fluorophenyl)ethyl)a- | | (m, 1H), 1.57 (d, J = | |
| | mino)pyrido[3,2- | | 7.2 Hz, 3H). | |
| | d]pyrimidin-4(3H)- | | | |
| | one | | | |
| Example 350 Method 6 Mixture of Stereo- isomers | 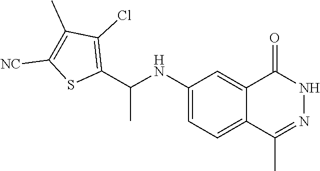 [SMILES: Cc1c(C#N)sc(C(C)Nc2ccc3c(C)n[nH]c(=O)c3c2)c1Cl] | White solid, yield: 42.7% | ¹H NMR (400 MHz, DMSO-d₆) δ 12.06 (s, 1H), 7.68 (d, J = 8.9 Hz, 1H), 7.14 (d, J = 8.4 Hz, 1H), 7.10 (s, 1H), 5.03-4.91 (m, 1H), 2.38 (s, 3H), 2.34 (s, 3H), 1.58 (d, J = 6.6 Hz, 3H) | 359.1 |
| | 4-chloro-3-methyl-5- | | | |
| | (1-((1-methyl-4-oxo- | | | |
| | 3,4- | | | |
| | dihydrophthalazin-6- | | | |
| | yl)amino)ethyl)thio- | | | |
| | phene-2-carbonitrile | | | |
| Example 351 Method 9 Mixture of Stereo- isomers | 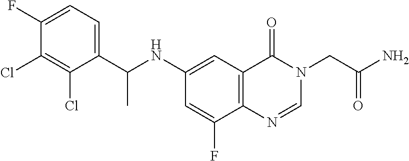 [SMILES: CC(Nc1cc(F)c2ncn(CC(N)=O)c(=O)c2c1)c1ccc(F)c(Cl)c1Cl] | white solid, yield: 46.7% | ¹H NMR (400 MHz, DMSO-d₆) δ 7.98 (s, 1H), 7.73-7.65 (m, 1H), 7.48-7.37 (m, 2H), 7.25 (d, J = 6.8 Hz, 2H), 6.92 (dd, J = 12.8, 2.4 Hz, 1H), 6.67 (d, J = 2.4 Hz, 1H), 4.86 (p, J = 6.8 Hz, 1H), 4.51 (s, 2H), 1.46 | 427.3 |
| | 2-(6-((1-(2,3- | | (d, J = 6.8 Hz, 3H). | |
| | dichloro-4- | | | |
| | fluorophenyl)ethyl)a- | | | |
| | mino)-8-fluoro-4- | | | |
| | oxoquinazolin- | | | |
| | 3(4H)-yl)acetamide | | | |
| Example 352 Method 6 Mixture of Stereo- isomers | 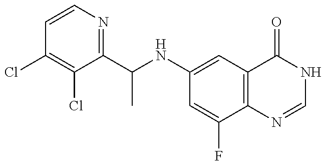 [SMILES: CC(Nc1cc(F)c2nc[nH]c(=O)c2c1)c1nccc(Cl)c1Cl] | White solid, yield: 45.1% | ¹H NMR (400 MHz, DMSO) δ 12.04 (s, 1H), 8.47 (d, J = 5.2 Hz, 1H), 7.78 (s, 1H), 7.66 (d, J = 5.2 Hz, 1H), 7.06-6.97 (m, 2H), 6.82 (d, J = 2.5 Hz, 1H), 5.20-5.09 (m, 1H), 1.48 (d, J = 6.8 Hz, 3H). | 353.1 |
| | 6-((1-(3,4- | | | |
| | dichloropyridin-2- | | | |
| | yl)ethyl)amino)-8- | | | |
| | fluoroquinazolin- | | | |
| | 4(3H)-one | | | |
| Example 353 Method 6 Mixture of Stereo- isomers | 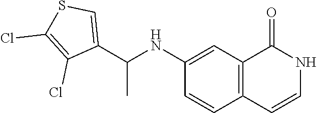 [SMILES: CC(Nc1ccc2cc[nH]c(=O)c2c1)c1csc(Cl)c1Cl] | White solid, yield: 5.8% | ¹H NMR (400 MHz, DMSO) δ 10.91 (d, J = 4.8 Hz, 1H), 7.37 (d, J = 8.5 Hz, 1H), 7.34 (s, 1H), 7.08 (d, J = 2.2 Hz, 1H), 7.03 (dd, J = 8.4, 2.4 Hz, 1H), 6.87- 6.78 (m, 1H), 6.61 (d, J = | 339.1 |
| | 7-((1-(4,5- | | 7.2 Hz, 1H), 6.34 (d, | |
| | dichlorothiophen-3- | | J = 6.8 Hz, 1H), 4.62- | |
| | yl)ethyl)amino)iso- | | 4.52 (m, 1H), 1.47 (d, J = | |
| | quinolin-1(2H)-one | | 6.8 Hz, 3H). | |
| Example 354 Method 6 Mixture of Stereo- isomers | 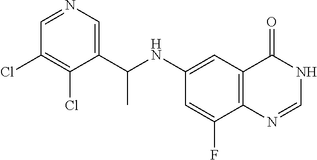 [SMILES: CC(Nc1cc(F)c2nc[nH]c(=O)c2c1)c1cncc(Cl)c1Cl] | White solid, yield: 13.5% | ¹H NMR (400 MHz, DMSO-d₆) δ 12.07 (s, 1H), 8.67 (s, 1H), 8.54 (s, 1H), 7.79 (d, J = 3.4 Hz, 1H), 7.23 (d, J = 7.5 Hz, 1H), 6.95 (dd, J = 12.6, 2.6 Hz, 1H), 6.70 (d, J = 2.5 Hz, 1H), 4.93 (p, J = 6.6 Hz, 1H), 1.53 (d, J = | 353.2 |
| | 6-((1-(4,5- | | 6.8 Hz, 3H). | |
| | dichloropyridin-3- | | | |
| | yl)ethyl)amino)-8- | | | |
| | fluoroquinazolin- | | | |
| | 4(3H)-one | | | |
| Example 382 Method 6 single unknown stero- isomer | 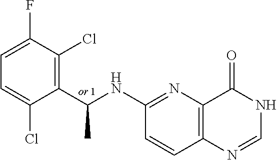 [SMILES: C[C@H](Nc1ccc2nc[nH]c(=O)c2n1)c1c(Cl)ccc(F)c1Cl] | White solid, yield: 35.1% | ¹H NMR (400 MHz, DMSO-d₆) δ 11.97 (s, 1H), 7.80-7.73 (m, 2H), 7.65 (d, J = 9.0 Hz, 1H), 7.39 (dd, J = 9.0, 5.1 Hz, 1H), 7.28 (t, J = 8.7 Hz, 1H), 7.04 (d, J = 8.9 Hz, 1H), 5.80 (p, J = 7.1 Hz, 1H), 1.59 (d, J = 7.3 | 353.1 Note; CHIRAL Cellulose SB, 2 cm*25 cm, 5 um, MeOH/DCM, MtBE, 20 mL/min, |
| | rel-(S)-6-((1-(2,6- | | Hz, 3H). | Rt = 4.671 |
| | dichloro-3- | | | min. |
| | fluorophenyl)ethyl)a- | | | |
| | mino)pyrido[3,2- | | | |
| | d]pyrimidin-4(3H)- | | | |
| | one | | | |
| Example 383 Method 6 single unknown stereo- isomer | 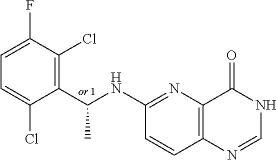 [SMILES: C[C@@H](Nc1ccc2nc[nH]c(=O)c2n1)c1c(Cl)ccc(F)c1Cl] | White solid, yield: 36.7% | ¹H NMR (400 MHz, DMSO-d₆) δ 11.97 (s, 1H), 7.80-7.73 (m, 2H), 7.65 (d, J = 9.0 Hz, 1H), 7.39 (dd, J = 9.0, 5.1 Hz, 1H), 7.28 (t, J = 8.7 Hz, 1H), 7.04 (d, J = 8.9 Hz, 1H), 5.80 (p, J = 7.1 Hz, 1H), 1.59 (d, J = 7.3 | 353.1 Note; CHIRAL Cellulose SB, 2 cm*25 cm, 5 um, MeOH/DCM, MtBE, 20 mL/min, |
| | Rel-(R)-6-((1-(2,6- | | Hz, 3H). | Rt = 3.440 |
| | dichloro-3- | | | min |
| | fluorophenyl)ethyl)a- | | | |
| | mino)pyrido[3,2- | | | |
| | d]pyrimidin-4(3H)- | | | |
| | one | | | |
| Example 384 Method 6 Mixture of Stereo- isomers | 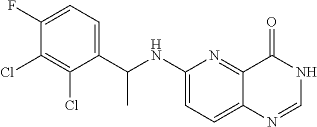 [SMILES: CC(Nc1ccc2nc[nH]c(=O)c2n1)c1ccc(F)c(Cl)c1Cl] | White solid, yield: 47.7% | ¹H NMR (400 MHz, DMSO-d₆) δ 12.11 (s, 1H), 7.86 (d, J = 7.6 Hz, 1H), 7.80 (d, J = 3.5 Hz, 1H), 7.69 (d, J = 8.9 Hz, 1H), 7.51 (dd, J = 8.9, 5.9 Hz, 1H), 7.41 (t, J = 8.7 Hz, 1H), 6.98 (d, J = | 353.15 |
| | 6-((1-(2,3-dichloro- | | 8.9 Hz, 1H), 5.59-5.41 | |
| | 4- | | (m, 1H), 1.45 (d, J = 6.9 | |
| | fluorophenyl)ethyl)a- | | Hz, 3H). | |
| | mino)pyrido[3,2- | | | |
| | d]pyrimidin-4(3H)- | | | |
| | one | | | |
| Example 385 Method 6 single unknown stereo- isomer | 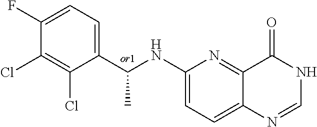 [SMILES: C[C@@H](Nc1ccc2nc[nH]c(=O)c2n1)c1ccc(F)c(Cl)c1Cl] | white solid, yield: 21.7% | ¹H NMR (400 MHz, DMSO-d6) δ 12.12 (s, 1H), 7.86 (d, J = 7.6 Hz, 1H), 7.80 (s, 1H), 7.69 (d, J = 8.9 Hz, 1H), 7.51 (dd, J = 8.8, 5.9 Hz, 1H), 7.41 (t, J = 8.7 Hz, 1H), 6.98 (d, J = | 353.15 Note: SFC CHIRALPAK AS-H 250 mm × 20 mm, 5 μm Modifier:IPA: 0.2% NH4OH = |
| | Rel-(R)-6-((1-(2,3- | | 8.9 Hz, 1H), 5.61- | 40:60 |
| | dichloro-4- | | 5.40 (m, 1H), 1.45 (d, J = | Rt = 1.8 min |
| | fluorophenyl)ethyl)a- | | 6.9 Hz, 3H). | |
| | mino)pyrido[3,2- | | | |
| | d]pyrimidin-4(3H)- | | | |
| | one | | | |
| Example 386 Method 6 single unknown stero- isomer | 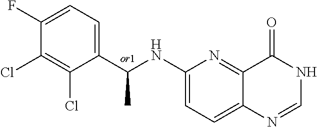 [SMILES: C[C@H](Nc1ccc2nc[nH]c(=O)c2n1)c1ccc(F)c(Cl)c1Cl] | white solid, yield: 24.5% | ¹H NMR (400 MHz, DMSO-d₆) δ 12.12 (s, 1H), 7.86 (d, J = 7.6 Hz, 1H), 7.80 (s, 1H), 7.69 (d, J = 8.9 Hz, 1H), 7.51 (dd, J = 8.9, 5.9 Hz, 1H), 7.41 (t, J = 8.7 Hz, 1H), 6.98 (d, J = | 353.15 Note: SFC CHIRALPAK AS-H 25 mm × 20 mm, 5 μm Modifier:IPA: 0.2% NH4OH = |
| | Rel-(S)-6-((1-(2,3- | | 9.0 Hz, 1H), 5.62- | 40:60 |
| | dichloro-4- | | 5.38 (m, 1H), 1.45 (d, J = | Rt = 2.6 min |
| | fluorophenyl)ethyl)a- | | 6.9 Hz, 3H). | |
| | mino)pyrido[3,2- | | | |
| | d]pyrimidin-4(3H)- | | | |
| | one | | | |
| Example 387 Method 6 single unknown steroisomer | 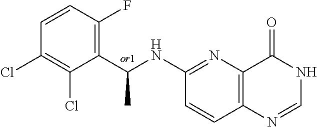 [SMILES: C[C@H](Nc1ccc2nc[nH]c(=O)c2n1)c1c(F)ccc(Cl)c1Cl] | White solid, yield: 21.4% | ¹H NMR (400 MHz, DMSO-d₆) δ 12.04 (s, 1H), 7.81-7.75 (m, 1H), 7.69 (d, J = 7.6 Hz, 1H), 7.65 (d, J = 8.8 Hz, 1H), 7.54 (dd, J = 8.8, 5.2 Hz, 1H), 7.22 (dd, J = 10.4, 8.8 Hz, | 355.3 Note: CHIRAL Cellulose SB 2 cm × 25 cm, 5 um Modifier:MtBE (0.1% |
| | Rel-(S)-6-((1-(2,3- | | 1H), 7.04 (d, J = 9.2 | DEA + 0.1% |
| | dichloro-6- | | Hz, 1H), 5.77 (q, J = | FA):(MeOH:DCM = |
| | fluorophenyl)ethyl)a- | | 7.2 Hz, 1H), 1.57 (d, J = | 1:1) = |
| | mino)pyrido[3,2- | | 7.2 Hz, 3H). | 85:15 |
| | d]pyrimidin-4(3H)- | | | Rt = 4.454 |
| | one | | | min |
| Example 388 Method 6 single unknown stereo- isomer | 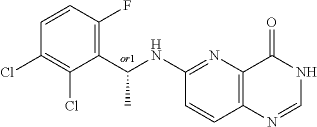 [SMILES: C[C@@H](Nc1ccc2nc[nH]c(=O)c2n1)c1c(F)ccc(Cl)c1Cl] | White solid, yield: 27.1% | ¹H NMR (400 MHz, DMSO-d₆) δ 12.04 (s, 1H), 7.79 (d, J = 3.2 Hz, 1H), 7.69 (d, J = 7.6 Hz, 1H), 7.65 (d, J = 8.8 Hz, 1H), 7.54 (dd, J = 8.8, 5.2 Hz, 1H), 7.22 (dd, J = 10.4, 8.8 | 355.3 Note: CHIRAL Cellulose SB 2 cm × 25 cm, 5 um Modifier:MtBE (0.1% |
| | Rel-(R)-6-((1-(2,3- | | Hz, 1H), 7.04 (d, J = | DEA + 0.1% |
| | dichloro-6- | | 8.8 Hz, 1H), 5.77 (q, J = | FA):(MeOH:DCM = |
| | fluorophenyl)ethyl)a- | | 7.2 Hz, 1H), 1.57 (d, | 1:1) = |
| | mino)pyrido[3,2- | | J = 7.2 Hz, 3H). | 85:15 |
| | d]pyrimidin-4(3H)- | | | Rt = 3.778 |
| | one | | | min |

### Preparation of 2,3-dichloro-N-(4-methyl-1-oxo-1,2-dihydroisoquinolin-7-yl)benzenesulfonamide (188) Method 7

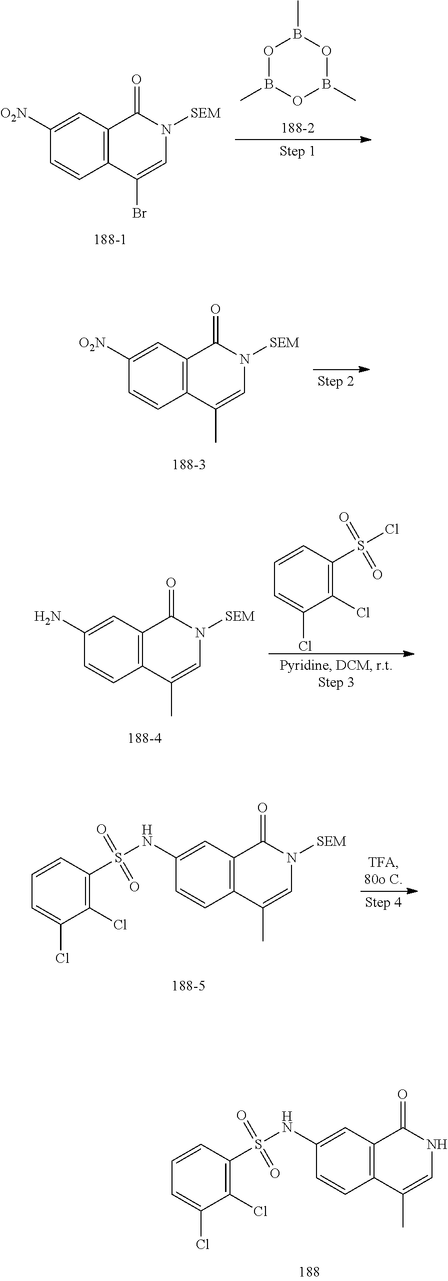

Step 1: A mixture of 188-1, 188-2 (470.36 mg, 3.747 mmol), K₂CO₃ (517.87 mg, 3.747 mmol), and Pd(dppf)Cl₂ (91.39 mg, 0.1249 mmol) in 1,4-Dioxane/H₂O (10:2 mL) was stirred for 16 hour at 100° C. under N₂. After cooling, the reaction mixture was diluted with water (60 mL), extracted three times with ethyl acetate (20 mL). Organic layers were combined, dried over anhydrous Na₂SO₄ and concentrated under reduced pressure to give a crude product that was purified through column chromatography on silica gel (PE/EA=3:1) to give 188-3 (245 mg, 58.48% yield) as a yellow solid. MS (ESI) m/z 335.0 [M+H]⁺.

Step 2: A mixture solution of 188-3 (125 mg, 0.3726 mmol), Fe (104.05 mg, 1.863 mmol), NH₄Cl (99.65 mg, 1.863 mmol) and EtOH/H₂O (10:10 mL) was stirred at 80° C. for 2 hrs. After cooling, the reaction mixture was diluted with water (60 mL), extracted three times with ethyl acetate (20 mL). The organic layers were combined, dried over anhydrous Na₂SO₄ and concentrated under reduced pressure to give a crude product that was purified through column chromatography on silica gel (PE/EA=4:1) to give 188-4 (102 mg, 89.61% yield) as oil. MS (ESI) m/z 305.0 [M+H]⁺.

Step 3: A mixture solution of 188-4 (92 mg, 0.3012 mmol), 2,3-dichlorobenzenesulfonyl chloride (73.95 mg, 0.3012 mmol), pyridine (71.47 mg, 0.9036 mmol) and DCM (10 mL) was stirred at 25° C. for 16 hrs. After the reaction was completed, the reaction mixture was diluted with water (60 mL) and extracted three times with ethyl acetate (20 mL). The organic layers were combined, dried over anhydrous Na₂SO₄ and concentrated under reduced pressure to give a crude product that was purified through column chromatography on silica gel (PE/EA=5:1) to give 188-5 (129 mg, 83.23% yield) as oil. MS (ESI) m/z 512.9 [M+H]⁺.

Step 4: A solution of 188-5 (119 mg, 0.2313 mmol) in TFA (10 mL) was stirred for 2 hour at 80° C. After reaction was completed, the reaction mixture was diluted with water (60 mL), extracted three times with DCM (20 mL). The organic layers were combined, dried over anhydrous Na₂SO₄ and concentrated under reduced pressure to give a crude product that was purified by perp-HPLC (column-Gemini-C18 150×21.2 mm, 5 um; Mobile phase: ACN-H₂O (0.1% FA), 40%-60%) to afford the desired product 188 (31.2 mg) as a white solid. MS (ESI) m/z 382.8 [M+H]⁺. ¹H NMR (400 MHz, DMSO-d₆) δ 11.16-11.03 (m, 2H), 8.05 (dd, J=8.0, 1.6 Hz, 1H), 7.97-7.88 (m, 2H), 7.59-7.50 (2, 3H), 6.90 (d, J=4.8 Hz, 1H), 2.13 (d, J=0.7 Hz, 3H).

Compound 189 was synthesized using a method similar to that used for Compound 188.

| Example | | Appear- | | |
|---|---|---|---|---|
| (Compound) | | ance | | |
| No. | | and | | MS (m/z) |
| Method | Structure and Name | Yield | ¹H NMR Data | [M + H]⁺ |
| Example 189 Method 7 Mixture of Stereoisomers | 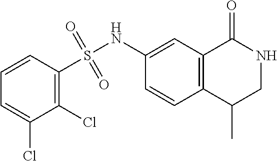 [SMILES: CC1CNC(=O)c2cc(NS(=O)(=O)c3cccc(Cl)c3Cl)ccc21] | white solid, yield: 4.5% | ¹H NMR (400 MHz, DMSO-d₆) δ 10.90 (s, 1H), 8.02 (dd, J = 8.0, 1.4 Hz, 1H), 7.93 (dd, J = 8.1, 1.4 Hz, 2H), 7.61- 7.54 (m, 2H), 7.23 (d, J = 2.0 Hz, 2H), 3.38 (dd, J = 4.8, 3.0 Hz, 1H), 3.04-2.92 (m, 2H), 1.15 (d, J = 6.8 Hz, 3H). | 385.2 |
| | 2,3-dichloro-N-(4- | | | |
| | methyl-1-oxo-1,2,3,4- | | | |
| | tetrahydroisoquinolin- | | | |
| | 7- | | | |
| | yl)benzenesulfonamide | | | |

### Preparation of 6-((1-(4,5-dichlorothiazol-2-yl)ethyl)amino)quinazolin-4(3H)-one (202) Method 8

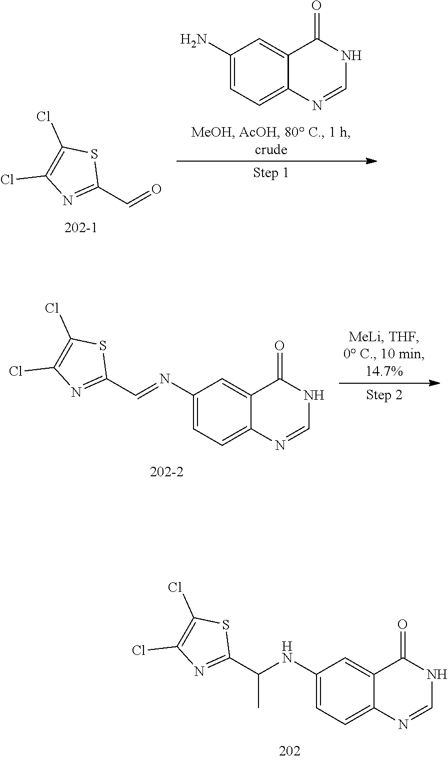 [SMILES: Nc1ccc2nc[nH]c(=O)c2c1 ; O=Cc1nc(Cl)c(Cl)s1 ; O=c1[nH]cnc2ccc(/N=C/c3nc(Cl)c(Cl)s3)cc12 ; CC(Nc1ccc2nc[nH]c(=O)c2c1)c1nc(Cl)c(Cl)s1]

Step 1: To a solution of 6-aminoquinazolin-4(3H)-one (88 mg, 0.54 mmol) in MeOH (5 mL) was added 202-1 (100 mg, 0.55 mmol) and AcOH (0.5 ml). The mixture was stirred at 80° C. for 1 hour. Water was added and the resulting mixture was extracted with EtOAc, dried over Na₂SO₄, and concentrated in vacuo to give (E)-6-(((4,5-dichlorothiazol-2-yl)methylene)amino)quinazolin-4(3H)-one 202-2(100 mg, crude) as a yellow solid without purification. Mass (m/z): 325.40 [M+H]⁺.

Step 2: To a solution of 203-2 (100 mg, 0.30 mmol) in TH (10 ml), was added Methyllithium (0.4 mL, 1.6 M) dropwise at 0° C. The mixture was stirred at 0° C. for 10 minutes. Water was added and the resulting mixture was extracted with EtOAc, dried over Na₂SO₄, and concentrated in vacuo. The crude product was purified by prep-HPLC (H₂O:ACN=30:70˜100:0, products collected at 60% ACN) to afford 202 (13.5 mg, 14.7% yield) as a white solid. Mass (m/z): 341.40 [M+H]⁺. ¹H NMR (400 MHz, DMSO-d₆) δ 11.97 (brs, 1H), 7.83 (s, 1H), 7.48 (d, J=8.8 Hz, 1H), 7.22 (dd, J=8.8, 2.8 Hz, 1H), 7.10-7.04 (m, 2H), 4.86 (p, J=6.4 Hz, 1H), 1.57 (d, J=6.8 Hz, 3H).

Compounds 187, 190, 207, 214, 216, 226A-226B, 232, 247-248, 256, 272,308,328,338 and 344-346 were synthesized using a method similar to that used for Compound 202.

| Example | | Appear- | | |
|---|---|---|---|---|
| (Compound) | | ance | | |
| No. | | and | | |
| Method | Structure and Name | Yield | ¹H NMR Data | [M + H]⁺ |
| Example 187 Method 8 Mixture of Stereoisomers | 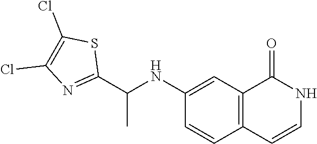 [SMILES: CC(Nc1ccc2cc[nH]c(=O)c2c1)c1nc(Cl)c(Cl)s1] | white solid, yield: 7.6% | ¹H NMR (400 MHz, DMSO-d₆) δ 10.95 (d, J = 5.6 Hz, 1H), 7.44 (d, J = 8.6 Hz, 1H), 7.21 (d, J = 2.6 Hz, 1H), 7.13 (dd, J = 8.6, 2.6 Hz, 1H), 6.92- 6.86 (m, 2H), 6.39 (d, J = 7.0 Hz, 1H), 4.84 (p, J = 6.6 Hz, 1H), 1.57 (d, J = | 340.1 |
| | 7-((1-(4,5- | | 6.8 Hz, 3H). | |
| | dichlorothiazol-2- | | | |
| | yl)ethyl)amino)isoquino- | | | |
| | lin-1(2H)-one | | | |
| Example 190 Method 8 Mixture of Stereoisomers | 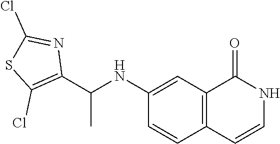 [SMILES: CC(Nc1ccc2cc[nH]c(=O)c2c1)c1nc(Cl)sc1Cl] | white solid, yield: 6.4% | ¹H NMR (400 MHz, DMSO-d₆) δ 10.85 (d, J = 5.5 Hz, 1H), 7.36 (d, J = 8.6 Hz, 1H), 7.23 (d, J = 2.6 Hz, 1H), 7.07 (dd, J = 8.6, 2.6 Hz, 1H), 6.83 (dd, J = 7.0, 5.6 Hz, 1H), 6.57 (d, J = 8.2 Hz, 1H), 6.33 (d, J = 6.9 Hz, | 340.2 |
| | 7-((1-(2,5- | | 1H), 4.77 (p, J = 7.0 Hz, | |
| | dichlorothiazol-4- | | 1H), 1.49 (d, J = 6.7 Hz, | |
| | yl)ethyl)amino)isoquino- | | 3H). | |
| | lin-1(2H)-one | | | |
| Example 207 Method 8 Mixture of Stereoisomers | 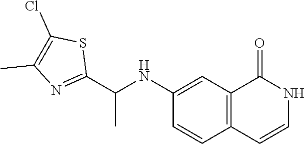 [SMILES: Cc1nc(C(C)Nc2ccc3cc[nH]c(=O)c3c2)sc1Cl] | white solid, yield: 15.3% | ¹H NMR (400 MHz, DMSO-d₆) δ 10.95 (d, J = 5.4 Hz, 1H), 7.42 (d, J = 8.6 Hz, 1H), 7.18 (d, J = 2.5 Hz, 1H), 7.11 (dd, J = 8.6, 2.6 Hz, 1H), 6.90-6.81 (m, 2H), 6.37 (d, J = 7.0 Hz, 1H), 4.76 (p, J = 6.7 Hz, 1H), 2.30 | 320.2 |
| | 7-((1-(5-chloro-4- | | (s, 3H), 1.55 (d, J = 6.8 | |
| | methylthiazol-2- | | Hz, 3H). | |
| | yl)ethyl)amino)isoquino- | | | |
| | lin-1(2H)-one | | | |
| Example 214 Method 8 Mixture of Stereoisomers | 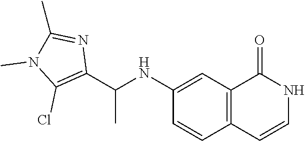 [SMILES: Cc1nc(C(C)Nc2ccc3cc[nH]c(=O)c3c2)c(Cl)n1C] | white solid, yield: 10.5% | ¹H NMR (400 MHz, DMSO-d₆) δ 10.83 (d, J = 5.5 Hz, 1H), 7.31 (d, J = 8.6 Hz, 1H), 7.23 (d, J = 2.5 Hz, 1H), 7.06 (dd, J = 8.6, 2.6 Hz, 1H), 6.80 (dd, J = 7.0, 5.3 Hz, 1H), 6.32 (d, J = 7.0 Hz, 1H), 6.28-6.19 (m, 1H), | 317.3 |
| | 7-((1-(5-chloro-1,2- | | 4.57-4.45 (m, 1H), 3.40 | |
| | dimethyl-1H- | | (s, 3H), 2.26 (s, 3H), | |
| | imidazol-4- | | 1.42 (d, J = 6.7 Hz, 3H). | |
| | yl)ethyl)amino)isoquino- | | | |
| | lin-1(2H)-one | | | |
| Example 216 Method 8 Mixture of Stereoisomers | 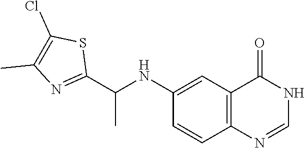 [SMILES: Cc1nc(C(C)Nc2ccc3nc[nH]c(=O)c3c2)sc1Cl] | white solid, yield: 21.1% | ¹H NMR (400 MHz, DMSO-d₆) δ 11.93 (s, 1H), 7.81 (s, 1H), 7.46 (d, J = 8.8 Hz, 1H), 7.20 (dd, J = 8.8, 2.8 Hz, 1H), 7.05 (d, J = 2.8 Hz, 1H), 6.98 (d, J = 6.0 Hz, 1H), 4.77 (p, J = 6.6 Hz, 1H), 2.30 (s, 3H), 1.55 (d, J = 6.7 Hz, 3H). | 321.2 |
| | 6-((1-(5-chloro-4- | | | |
| | methylthiazol-2- | | | |
| | yl)ethyl)amino)quinazo- | | | |
| | lin-4(3H)-one | | | |
| Example 226A Method 8 single unknown stereoisomer | 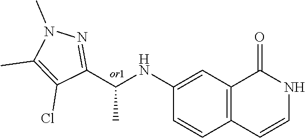 [SMILES: Cc1c(Cl)c([C@@H](C)Nc2ccc3cc[nH]c(=O)c3c2)nn1C] | Light yellow solid | ¹H NMR (400 MHz, DMSO- d₆) δ 10.84 (d, J = 5.6 Hz, 1H), 7.32 (d, J = 8.6 Hz, 1H), 7.22 (d, J = 2.5 Hz, 1H), 7.07 (dd, J = 8.6, 2.6 Hz, 1H), 6.80 (dd, J = 7.1, 5.6 Hz, 1H), 6.39- 6.21 (m, 2H), 4.62 (t, J = 7.1 Hz, 1H), 3.68 (s, 3H), 2.16 (s, 3H), 1.47 (d, J = 6.7 Hz, | 317.2 |
| | rel-(S)-7-((1-(4- | | 3H). | |
| | chloro-1,5-dimethyl- | | | |
| | 1H-pyrazol-3- | | | |
| | yl)ethyl)amino)isoquino- | | | |
| | lin-1(2H)-one | | | |
| Example 226B Method 8 single unknown stereoisomer | 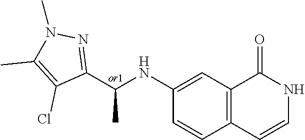 [SMILES: Cc1c(Cl)c([C@H](C)Nc2ccc3cc[nH]c(=O)c3c2)nn1C] | white solid, yield: 31.2% | ¹H NMR (400 MHz, DMSO-d₆) δ 10.84 (d, J = 5.7 Hz, 1H), 7.32 (d, J = 8.6 Hz, 1H), 7.22 (d, J = 2.5 Hz, 1H), 7.07 (dd, J = 8.6, 2.6 Hz, 1H), 6.80 (dd, J = 7.1, 5.7 Hz, 1H), 6.35-6.28 (m, 2H), 4.61 (q, J = 6.8 Hz, | 317.2 |
| | rel-(R)-7-((1-(4- | | 1H), 3.68 (s, 3H), 2.16 | |
| | chloro-1,5-dimethyl- | | (s, 3H), 1.47 (d, J = 6.8 | |
| | 1H-pyrazol-3- | | Hz, 3H). | |
| | yl)ethyl)amino)isoquino- | | | |
| | lin-1(2H)-one | | | |
| Example 232 Method 8 Mixture of Stereoisomers | 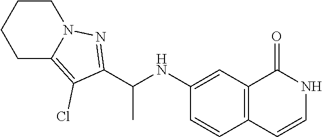 [SMILES: CC(Nc1ccc2cc[nH]c(=O)c2c1)c1nn2c(c1Cl)CCCC2] | white solid, yield: 11.2% | ¹H NMR (400 MHz, DMSO-d6) δ 10.86 (d, J = 5.6 Hz, 1H), 7.33 (d, J = 8.6 Hz, 1H), 7.23 (d, J = 2.5 Hz, 1H), 7.08 (dd, J = 8.6, 2.6 Hz, 1H), 6.81 (dd, J = 7.0, 5.6 Hz, 1H), 6.33 (d, J = 7.4 Hz, 2H), 4.65 (p, J = 6.9 Hz, | 343.1 |
| | 7-((1-(3-chloro- | | 1H), 3.97 (td, J = 6.0, 2.3 | |
| | 4,5,6,7- | | Hz, 2H), 2.60 (t, J = 6.4 | |
| | tetrahydropyrazolo[1,5- | | Hz, 2H), 1.96-1.87 (m, | |
| | a]pyridin-2- | | 2H), 1.83-1.70 (m, | |
| | yl)ethyl)amino)isoquino- | | 2H), 1.48 (d, J = 6.7 Hz, | |
| | lin-1(2H)-one | | 3H). | |
| Example 247 Method 8 Mixture of Stereoisomers | 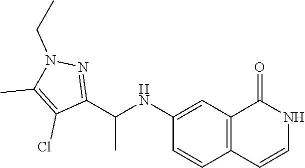 [SMILES: CCn1nc(C(C)Nc2ccc3cc[nH]c(=O)c3c2)c(Cl)c1C] | white solid, yield: 20.3% | ¹H NMR (400 MHz, Chloroform-d) δ 9.13 (s, 1H), 7.58 (d, J = 2.5 Hz, 1H), 7.36 (d, J = 8.6 Hz, 1H), 7.08 (dd, J = 8.6, 2.5 Hz, 1H), 6.86-6.80 (m, 1H), 6.43 (d, J = 7.1 Hz, 1H), 4.87 (q, J = 6.7 Hz, 1H), 4.03 (q, J = 7.2 Hz, 2H), 2.20 (s, 3H), 1.59 (d, J = 6.7 Hz, 3H), | 331.3 |
| | 7-((1-(4-chloro-1- | | 1.36 (t, J = 7.3 Hz, 3H). | |
| | ethyl-5-methyl-1H- | | | |
| | pyrazol-3- | | | |
| | yl)ethyl)amino)isoquino- | | | |
| | lin-1(2H)-one | | | |
| Example 248 Method 8 Mixture of Stereoisomers | 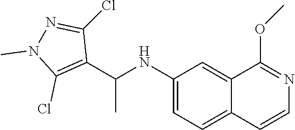 [SMILES: COc1nccc2ccc(NC(C)c3c(Cl)nn(C)c3Cl)cc12] | white solid, yield: 18.3% | ¹H NMR (400 MHz, DMSO-d₆) δ 7.64 (d, J = 5.7 Hz, 1H), 7.56 (d, J = 8.9 Hz, 1H), 7.19 (dd, J = 8.8, 2.4 Hz, 1H), 7.10 (d, J = 5.7 Hz, 1H), 6.89 (d, J = 2.4 Hz, 1H), 6.67 (d, J = 6.7 Hz, 1H), 4.51 | 351.1 |
| | N-(1-(3,5-dichloro-1- | | (p, J = 6.9 Hz, 1H), 3.96 | |
| | methyl-1H-pyrazol-4- | | (s, 3H), 3.70 (s, 3H), | |
| | yl)ethyl)-1- | | 1.54 (d, J = 6.9 Hz, 3H). | |
| | methoxyisoquinolin- | | | |
| | 7-amine | | | |
| Example 256 Method 8 Mixture of Stereoisomers | 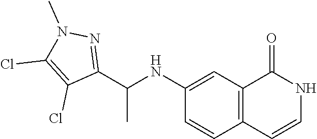 [SMILES: CC(Nc1ccc2cc[nH]c(=O)c2c1)c1nn(C)c(Cl)c1Cl] | white solid, yield: 15.5% | ¹H NMR (400 MHz, DMSO-d₆) δ 10.86 (d, J = 5.5 Hz, 1H), 7.34 (d, J = 8.6 Hz, 1H), 7.23-7.18 (m, 1H), 7.07 (dd, J = 8.6, 2.6 Hz, 1H), 6.81 (t, J = 6.3 Hz, 1H), 6.44 (d, J = 7.6 Hz, 1H), 6.33 (d, J = 7.0 Hz, 1H), 4.66 (p, | 337.2 |
| | 7-((1-(4,5-dichloro-1- | | J = 6.9 Hz, 1H), 3.77 (s, | |
| | methyl-1H-pyrazol-3- | | 2H), 1.50 (d, J = 6.8 Hz, | |
| | yl)ethyl)amino)isoquino- | | 3H). | |
| | lin-1(2H)-one | | | |
| Example 272 Method 8 Mixture of Stereoisomers | 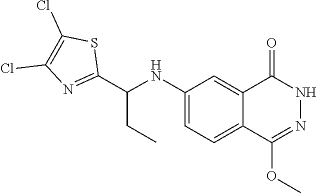 [SMILES: CCC(Nc1ccc2c(OC)n[nH]c(=O)c2c1)c1nc(Cl)c(Cl)s1] | white solid, yield: 13.4% | ¹H NMR (400 MHz, Methanol-d₄) δ 7.81 (d, J = 8.7 Hz, 1H), 7.30 (d, J = 2.5 Hz, 1H), 7.26 (dd, J = 8.8, 2.5 Hz, 1H), 4.70 (dd, J = 8.4, 5.2 Hz, 1H), 3.93 (s, 3H), 2.15- 1.95 (m, 2H), 1.10 (t, J = 7.4 Hz, 3H). | 385.03 |
| | 7-((1-(4,5- | | | |
| | dichlorothiazol-2- | | | |
| | yl)propyl)amino)-4- | | | |
| | methoxyphthalazin- | | | |
| | 1(2H)-one | | | |
| Example 308 Method 8 Mixture of Stereoisomers | 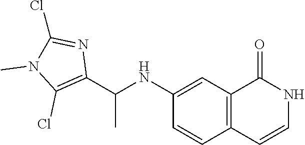 [SMILES: CC(Nc1ccc2cc[nH]c(=O)c2c1)c1nc(Cl)n(C)c1Cl] | brown solid, yield: 10.7% | ¹H NMR (400 MHz, CDCl₃) δ 9.62 (s, 1H), 7.56 (s, 1H), 7.34 (d, J = 8.6 Hz, 1H), 7.02 (dd, J = 8.5, 2.3 Hz, 1H), 6.86 (s, 1H), 6.41 (d, J = 7.1 Hz, 1H), 4.80 (d, J = 6.8 Hz, 1H), 4.59 (s, 1H), 3.51 (s, 3H), 1.53 (d, J = 6.5 Hz, 3H). | 352.8 |
| | 7-((1-(2,5-dichloro-1- | | | |
| | methyl-1H-imidazol- | | | |
| | 4- | | | |
| | yl)ethyl)amino)isoquino- | | | |
| | lin-1(2H)-one | | | |
| Example 328 Method 8 Mixture of Stereoisomers | 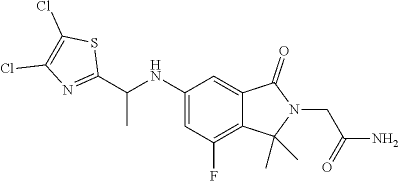 [SMILES: CC(Nc1cc(F)c2c(c1)C(=O)N(CC(N)=O)C2(C)C)c1nc(Cl)c(Cl)s1] | white solid, yield: 6.7% | ¹H NMR (400 MHz, Methanol-d₄) δ 6.77 (d, J = 1.9 Hz, 1H), 6.62 (dd, J = 11.9, 2.0 Hz, 1H), 4.75 (q, J = 6.8 Hz, 1H), 4.11 (d, J = 2.5 Hz, 2H), 1.62 (d, J = 6.8 Hz, 3H), 1.51 (s, 6H) | 432.1 |
| | 2-(5-((1-(4,5- | | | |
| | dichlorothiazol-2- | | | |
| | yl)ethyl)amino)-7- | | | |
| | fluoro-1,1-dimethyl-3- | | | |
| | oxoisoindolin-2- | | | |
| | yl)acetamide | | | |
| Example 338 Method 8 Mixture of Stereoisomers | 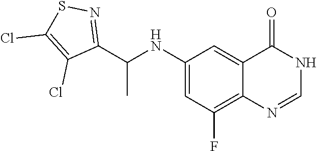 [SMILES: CC(Nc1cc(F)c2nc[nH]c(=O)c2c1)c1nsc(Cl)c1Cl] | white solid, yield: 18% | ¹H NMR (400 MHz, DMSO-d₆) δ 12.05 (s, 1H), 7.80 (d, J = 3.3 Hz, 1H), 7.06-6.96 (m, 2H), 6.88 (d, J = 2.6 Hz, 1H), 4.89 (t, J = 6.9 Hz, 1H), 1.54 (d, J = 6.8 Hz, 3H). | 359.1 |
| | 6-((1-(4,5- | | | |
| | dichloroisothiazol-3- | | | |
| | yl)ethyl)amino)-8- | | | |
| | fluoroquinazolin- | | | |
| | 4(3H)-one | | | |
| Example 344 Method 8 Mixture of Stereoisomers | 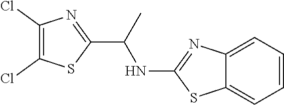 [SMILES: CC(Nc1nc2ccccc2s1)c1nc(Cl)c(Cl)s1] | yellow solid, yield: 8.8% | ¹H NMR (400 MHz, DMSO-d₆) δ 8.88 (d, J = 6.6 Hz, 1H), 7.74 (d, J = 7.8 Hz, 1H), 7.43 (d, J = 8.0 Hz, 1H), 7.29-7.21 (m, 1H), 7.08 (t, J = 7.6 Hz, 1H), 5.32 (t, J = 6.8 | 330.0 |
| | N-(1-(4,5- | | Hz, 1H), 1.59 (d, J = 7.0 | |
| | dichlorothiazol-2- | | Hz, 3H). | |
| | yl)ethyl)benzo[d]thiazol- | | | |
| | 2-amine | | | |
| Example 345 Method 8 Mixture of Stereoisomers | 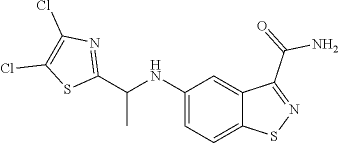 [SMILES: CC(Nc1ccc2snc(C(N)=O)c2c1)c1nc(Cl)c(Cl)s1] | yellow solid, yield: 12.4% | ¹H NMR (400 MHz, DMSO-d₆) δ 8.06-8.01 (m, 1H), 7.98 (d, J = 8.9 Hz, 1H), 7.85 (d, J = 2.2 Hz, 1H), 7.60 (s, 1H), 7.13 (dd, J = 8.9, 2.3 Hz, 1H), 7.00 (d, J = 5.8 Hz, 1H), 4.81 (p, J = 6.6 Hz, 1H), 1.58 (d, J = 6.6 Hz, | 372.9 |
| | 5-[1-(4,5- | | 3H). | |
| | dichlorothiazol-2- | | | |
| | yl)ethylamino]-1,2- | | | |
| | benzothiazole-3- | | | |
| | carboxamide | | | |
| Example 346 Method 8 Mixture of Stereoisomers | 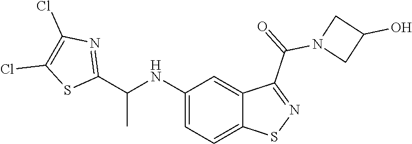 [SMILES: CC(Nc1ccc2snc(C(=O)N3CC(O)C3)c2c1)c1nc(Cl)c(Cl)s1] | yellow solid, yield: 14.5% | ¹H NMR (400 MHz, DMSO) δ 7.99 (d, J = 8.8 Hz, 1H), 7.74 (s, 1H), 7.14 (d, J = 9.0 Hz, 1H), 7.00 (s, 1H), 5.75 (d, J = 6.4 Hz, 1H), 4.80 (t, J = 6.1 Hz, 1H), 4.67 (d, J = 9.0 Hz, 1H), 4.51 (s, 1H), 4.31-4.18 (m, | 428.9 |
| | (5-((1-(4,5- | | 2H), 3.85-3.77 (m, | |
| | dichlorothiazol-2- | | 1H), 1.57 (d, J = 6.7 Hz, | |
| | yl)ethyl)amino)benzo[d] | | 3H). | |
| | isothiazol-3-yl)(3- | | | |
| | hydroxyazetidin-1- | | | |
| | yl)methanone | | | |

### Preparation of 2,3-dichloro-N-(1-(2-hydroxyethyl)-3,3-dimethyl-2-oxoindolin-6-yl)benzenesulfonamide (240) Method 9

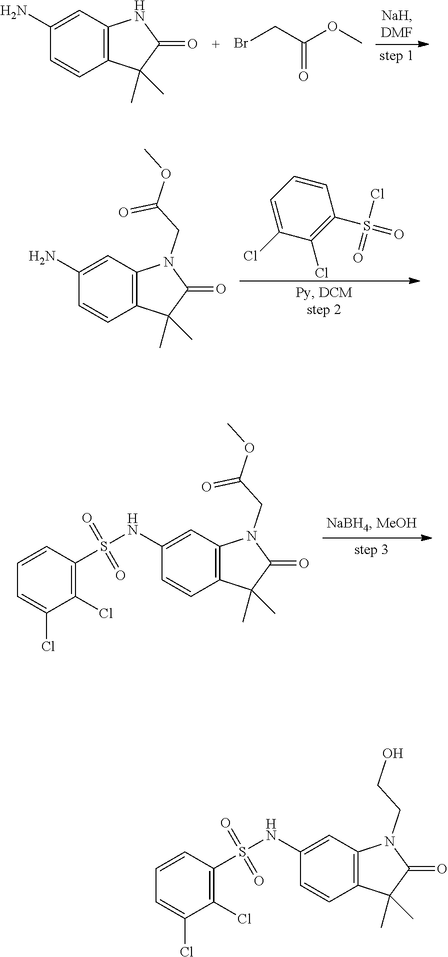

Step 1: To a solution of methyl 2-bromoacetate (1.00 eq, 153 mg, 0.999 mmol) in DMF (2 mL) was added sodium hydride (60%; 1.00 eq, 40 mg, 0.999 mmol) at 0° C. and then methyl 2-bromoacetate (1.00 eq, 153 mg, 0.999 mmol) was added. The reaction was stirred for 2 h at the same temperature. The reaction was then quenched with 0.5N HCl and extracted with ethyl acetate twice. The combined organic layers were washed with brine twice, dried over sodium sulfate and concentrated under vacuum to afford methyl 2-(6-amino-3,3-dimethyl-2-oxo-indolin-1-yl)acetate (240 mg, 0.97 mmol, 96.8% yield) as a yellow oil. Mass (m/z): 249.2 [M+H]⁺.

Step 2: Methyl 2-[6-[(2,3-dichlorophenyl)sulfonylamino]-3,3-dimethyl-2-oxo-indolin-1-yl]acetate (400 mg, 0.875 mmol, 87.56% yield) was obtained as a brown solid using Method 1. ¹H NMR (400 MHz, DMSO-d₆) δ 10.80 (s, 1H), 8.05 (dd, J=8.0, 1.5 Hz, 1H), 7.94 (dd, J=8.1, 1.5 Hz, 1H), 7.55 (t, J=8.0 Hz, 1H), 7.22 (d, J=8.0 Hz, 1H), 6.73 (dd, J=7.9, 1.9 Hz, 1H), 6.64 (d, J=1.9 Hz, 1H), 4.47 (s, 2H), 3.67 (s, 3H), 1.21 (s, 6H).

Step 3: To a solution of methyl 2-[6-[(2,3-dichlorophenyl)sulfonylamino]-3,3-dimethyl-2-oxo-indolin-1-yl]acetate (1.00 eq, 70 mg, 0.15 mmol) in methanol (5 mL) was added NaBH₄ (3.00 eq, 17 mg, 0.459 mmol) and stirred overnight at 60° C. The reaction was quenched with water, extracted with ethyl acetate twice. The combined organic layers were washed with brine, dried over sodium sulfate, and concentrated under vacuum. The residue was purified by Prep-HPLC to afford 2,3-dichloro-N-[1-(2-hydroxyethyl)-3,3-dimethyl-2-oxo-indolin-6-yl]benzenesulfonamide (11 mg, 0.025 mmol, 16.1% yield) as a white solid. Mass (m/z): 429.3 [M+H]⁺, ¹H NMR (400 MHz, DMSO-d₆) δ 10.99 (s, 1H), 8.07 (dd, J=7.9, 1.5 Hz, 1H), 7.90 (dd, J=8.1, 1.5 Hz, 1H), 7.53 (t, J=8.0 Hz, 1H), 7.13 (d, J=8.0 Hz, 1H), 6.78 (d, J=2.0 Hz, 1H), 6.67 (dd, J=8.0, 1.9 Hz, 1H), 4.84 (s, 1H), 3.60 (t, J=6.0 Hz, 2H), 3.49 (t, J=4.9 Hz, 2H), 1.17 (s, 6H).

Compounds 273, 289-290, 297, 300-301, 303, 305-307, 312, 312A, 312B, 317, 320, 320A, 323, 323A, 327, 329, 331-332, 334-337, 339-341, 341A, 341B, 343, 347-348, 347A, 355, 355A, 355B, 357-358, 361-368, 376-381 and 389-391 were synthesized using a method similar to that used for Compound 240.

| Example | | Appear- | | |
|---|---|---|---|---|
| (Compound) | | ance | | |
| No. | | and | | MS (m/z) |
| Method | Structure and Name | Yield | ¹H NMR Data | [M + H]⁺ |
| Example 273 Method 9 | 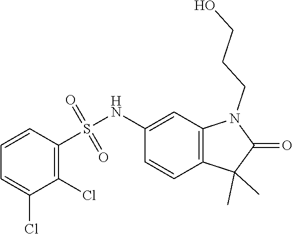 [SMILES: CC1(C)C(=O)N(CCCO)c2cc(NS(=O)(=O)c3cccc(Cl)c3Cl)ccc21] | white solid, yield: 16.1% | ¹H NMR (400 MHz, Methanol-d₄) δ 8.01 (dd, J = 8.0, 1.5 Hz, 1H), 7.70 (dd, J = 8.1, 1.5 Hz, 1H), 7.36 (t, J = 8.0 Hz, 1H), 7.06 (d, J = 8.0 Hz, 1H), 6.80 (d, J = 1.8 Hz, 1H), 6.75 (dd, J = 8.0, 1.9 Hz, 1H), 3.68 (t, J = 7.0 Hz, 2H), 3.49 (t, J = 6.3 Hz, 2H), 1.77-1.66 (m, 2H), 1.19 (s, 6H). | 443.1 |
| Example 289 Method 9 | 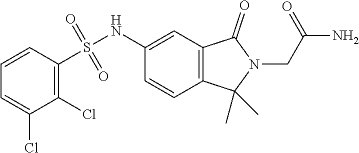 [SMILES: CC1(C)c2ccc(NS(=O)(=O)c3cccc(Cl)c3Cl)cc2C(=O)N1CC(N)=O] | white solid, yield: 55.9% | ¹H NMR (400 MHz, Methanol-d₄) δ 7.98 (dd, J = 7.9, 1.2 Hz, 1H), 7.65 (dd, J = 8.1, 1.2 Hz, 1H), 7.44 (d, J = 1.7 Hz, 1H), 7.39-7.27 (m, 3H), 4.02 (s, 2H), 1.32 (s, 6H). | 442.0 |
| | 2-(5-((2,3- | | | |
| | dichlorophenyl)sulfon- | | | |
| | amido)-1,1-dimethyl- | | | |
| | 3-oxoisoindolin-2- | | | |
| | yl)acetamide | | | |
| Example 290 Method 9 Mixture of Stereoisomers | 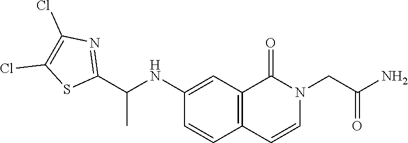 [SMILES: CC(Nc1ccc2ccn(CC(N)=O)c(=O)c2c1)c1nc(Cl)c(Cl)s1] | Yellow solid, yield: 55.6% | ¹HNMR (400 MHz, Methanol-d₄) δ 7.48 (d, J = 8.8 Hz, 1H), 7.33 (d, J = 2.4 Hz, 1H), 7.18 (dd, J = 2.4 Hz, J = 8.4Hz, 1H), 7.07 (d, J = 7.6 Hz, 1H), 6.59 (d, J = 7.2 Hz, 1H), 4.82-4.79 (m, 1H), 4.71-4.62 (m, 2H), 1.66 (d, J = 6.8 Hz, 3H). | 396.8 |
| | 2-(7-((1-(4,5- | | | |
| | dichlorothiazol-2- | | | |
| | yl)ethyl)amino)-1- | | | |
| | oxoisoquinolin-2(1H)- | | | |
| | yl)acetamide | | | |
| Example 297 Method 9 Mixture of Stereoisomers | 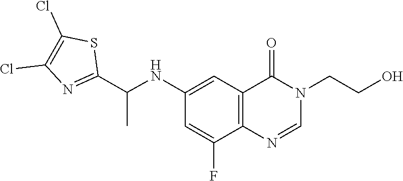 [SMILES: CC(Nc1cc(F)c2ncn(CCO)c(=O)c2c1)c1nc(Cl)c(Cl)s1] | White solid, yield: 7.4% | ¹H NMR (400 MHz, DMSO-d₆) δ 8.03 (s, 1H), 7.26 (d, J = 6.4 Hz, 1H), 7.10 (dd, J = 12.6, 2.5 Hz, 1H), 6.99 (d, J = 2.6 Hz, 1H), 4.95 (p, J = 6.6 Hz, 1H), 3.98 (t, J = 5.3 Hz, 2H), 3.63 (t, J = 5.3 Hz, 2H), 1.57 (d, J = 6.7 Hz, 3H). | 403.8 |
| | 6-((1-(4,5- | | | |
| | dichlorothiazol-2- | | | |
| | yl)ethyl)amino)-8- | | | |
| | fluoro-3-(2- | | | |
| | hydroxyethyl)quinazolin- | | | |
| | 4(3H)-one | | | |
| Example 300 Method 9 Mixture of Stereoisomers | 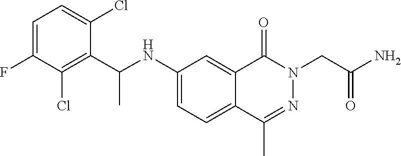 [SMILES: Cc1nn(CC(N)=O)c(=O)c2cc(NC(C)c3c(Cl)ccc(F)c3Cl)ccc12] | Light yellow solid, yield: 14.3% | ¹H NMR (400 MHz, DMSO-d₆) δ 7.63 (d, J = 8.8 Hz, 1H), 7.49 (d, J = 7.2 Hz, 1H), 7.44 (s, 1H), 7.36 (t, J = 8.8 Hz, 1H), 7.14-7.01 (m, 3H), 5.32 (t, J = 7.2 Hz, 1H), 4.52 (s, 2H), 2.35 (s, 3H), 1.64 (d, J = 7.2 Hz, 3H). | 423.1/425.1 |
| | 2-(7-((1-(2,6-dichloro- | | | |
| | 3- | | | |
| | fluorophenyl)ethyl) | | | |
| | amino)-4-methyl-1- | | | |
| | oxophthalazin-2(1H)- | | | |
| | yl)acetamide | | | |
| Example 301 Method 9 Mixture of Stereoisomers | 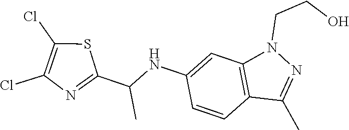 [SMILES: Cc1nn(CCO)c2cc(NC(C)c3nc(Cl)c(Cl)s3)ccc12] | white solid, yield: 9.74% | ¹H NMR (400 MHz, CDCl₃) δ 7.52 (d, J = 8.8 Hz, 1H), 6.66-6.61 (m, 1H), 6.31 (d, J = 1.6 Hz, 1H), 4.97-4.76 (m, 1H), 4.53-4.36 (m, 2H), 4.12-3.83 (m, 2H), 2.60 (s, 3H), 1.74 (d, J = 6.8 Hz, 3H). | 372.8 |
| | 2-(6-((1-(4,5- | | | |
| | dichlorothiazol-2- | | | |
| | yl)ethyl)amino)-3- | | | |
| | methyl-1H-indazol-1- | | | |
| | yl)ethan-1-ol | | | |
| Example 303 Method 9 | 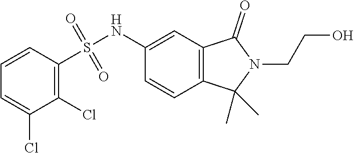 [SMILES: CC1(C)c2ccc(NS(=O)(=O)c3cccc(Cl)c3Cl)cc2C(=O)N1CCO] | white solid, yield: 14.9% | ¹H NMR (400 MHz, DMSO-d₆) δ 11.04 (s, 1H), 8.06 (dd, J = 8.0, 1.5 Hz, 1H), 7.94 (dd, J = 8.1, 1.5 Hz, 1H), 7.60- 7.52 (m, 2H), 7.35- 7.29 (m, 2H), 4.69 (d, J = 50.3 Hz, 1H), 3.54 (d, J = 6.7 Hz, 2H), 3.39 (d, J = 6.7 Hz, 2H), 1.36 (s, 6H). | 429.0 |
| Example 305 Method 9 Mixture of Stereoisomers | 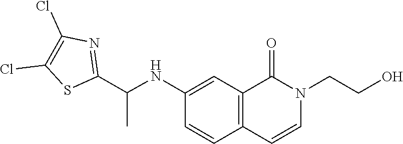 [SMILES: CC(Nc1ccc2ccn(CCO)c(=O)c2c1)c1nc(Cl)c(Cl)s1] | white solid, yield: 52.6% | ¹HNMR (400 MHz, CDCl₃) δ 7.71 (s, 1H), 7.42 (d, J = 8.4 Hz, 1H), 7.13-7.07 (m, 1H), 6.97 (d, J = 7.2 Hz, 1H), 6.48 (d, J = 7.2 Hz, 1H), 4.88 (q, J = 6.8 Hz, J = 13.6 Hz, 1H), 4.20-4.18 (m, 2H), 3.99 (t, J = 5.2 Hz, 2H), 1.74 (d, J = 6.8 Hz, 3H). | 383.8 |
| | 7-((1-(4,5- | | | |
| | dichlorothiazol-2- | | | |
| | yl)ethyl)amino)-2-(2- | | | |
| | hydroxyethyl)isoquinolin- | | | |
| | 1(2H)-one | | | |
| Example 306 Method 9 | 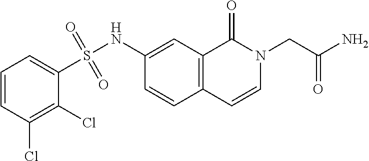 [SMILES: NC(=O)Cn1ccc2ccc(NS(=O)(=O)c3cccc(Cl)c3Cl)cc2c1=O] | White solid, yield: 67.3% | ¹HNMR (400 MHz, DMSO-d₆) δ 11.10 (s, 1H), 8.04 (d, J = 7.2 Hz, 1H), 7.90 (d, J = 6.8 Hz, 2H), 7.56-7.52 (m, 3H), 7.49-7.48 (m, 1H), 7.27 (d, J = 7.2 Hz, 1H), 7.18 (s, 1H), 6.49 (d, J = 7.2 Hz, 1H), 4.51 (s, 2H). | 425.8 |
| | 2-(7-((2,3- | | | |
| | dichlorophenyl) | | | |
| | sulfonamido)-1- | | | |
| | oxoisoquinolin-2(1H)- | | | |
| | yl)acetamide | | | |
| Example 307 Method 9 Mixture of Stereoisomers | 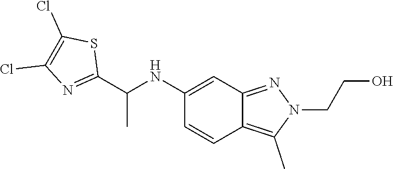 [SMILES: Cc1c2ccc(NC(C)c3nc(Cl)c(Cl)s3)cc2nn1CCO] | white solid, yield: 11.26% | ¹H NMR (400 MHz, CDCl₃) δ 7.43 (d, J = 8.8 Hz, 1H), 6.52-6.56 (m, 1H), 6.53 (s, 1H), 4.79 (d, J = 6.8 Hz, 1H), 4.53- 4.46 (m, 2H), 4.42 (s, 1H), 4.10 (t, J = 4.8 Hz, 2H), 2.58 (s, 3H), 1.69 (d, J = 6.4 Hz, 3H). | 372.8 |
| | 2-(6-((1-(4,5- | | | |
| | dichlorothiazol-2- | | | |
| | yl)ethyl)amino)-3- | | | |
| | methyl-2H-indazol-2- | | | |
| | yl)ethan-1-ol | | | |
| Example 312A Method 9 Mixture of stereoisomers | 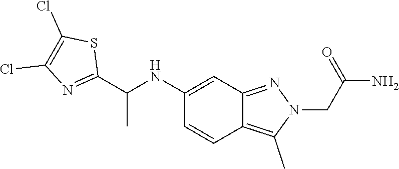 [SMILES: Cc1c2ccc(NC(C)c3nc(Cl)c(Cl)s3)cc2nn1CC(N)=O] | White solid, yield: 55.91% | ¹H NMR (400 MHz, CDCl₃) δ 7.46 (d, J = 9.2 Hz, 1H), 7.06 (d, 1H), 6.68-6.58 (m, 1H), 6.50 (d, J = 1.6 Hz, 1H), 5.48 (s, 1H), 5.07 (s, 2H), 4.86-4.73 (m, 1H), 4.44 (s, 1H), 2.63 (s, 3H), 1.72 (d, J = 6.8 Hz, 3H). | 383.8, 385.9 |
| | 2-(6-((1-(4,5- | | | |
| | dichlorothiazol-2- | | | |
| | yl)ethyl)amino)-3- | | | |
| | methyl-2H-indazol-2- | | | |
| | yl)acetamide | | | |
| Example 312 Method 9 single unknown stereoisomer | 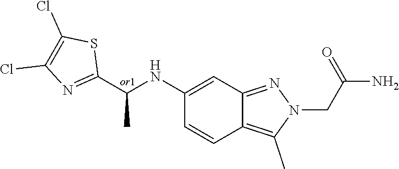 [SMILES: Cc1c2ccc(N[C@@H](C)c3nc(Cl)c(Cl)s3)cc2nn1CC(N)=O] | White solid, yield: 23% | ¹H NMR (400 MHz, CDCl₃) δ 7.46 (d, J = 9.2 Hz, 1H), 7.06 (d, 1H), 6.68-6.58 (m, 1H), 6.50 (d, J = 1.6 Hz, 1H), 5.48 (s, 1H), 5.07 (s, 2H), 4.86-4.73 (m, 1H), 4.44 (s, 1H), 2.63 (s, 3H), 1.72 (d, J = 6.8 Hz, 3H). | 383.8, 385.9 Note: CHIRALPAK IC, 2 cm*25 cm, 5 um, EtOH, MtBE, 20 mL/min, Rt = 5.78 min. |
| | rel-(R)-2-(6-((1-(4,5- | | | |
| | dichlorothiazol-2- | | | |
| | yl)ethyl)amino)-3- | | | |
| | methyl-2H-indazol-2- | | | |
| | yl)acetamide | | | |
| Example 312B Method 9 single unknown stereoisomer | 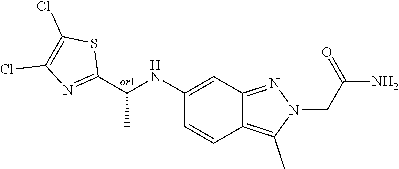 [SMILES: Cc1c2ccc(N[C@H](C)c3nc(Cl)c(Cl)s3)cc2nn1CC(N)=O] | yellow solid., yield: 23.53% | ¹H NMR (400 MHz, CDCl₃) δ 7.43 (d, J = 9.2 Hz, 1H), 6.84 (s, 1H), 6.63-6.58 (m, 1H), 6.46 (d, J = 2.0 Hz, 1H), 5.47 (s, 1H), 5.00 (s, 2H), 4.82-4.72 (m, 1H), 4.38 (s, 1H), 2.59 (s, 3H), 1.69 (d, J = 6.8 Hz, 3H). | 383.8, 385.8 CHIRALPAK IC, 2 cm*25 cm, 5 um, EtOH, MtBE, 20 mL/min, Rt = 3.0 min |
| | rel-(S)-2-(6-((1-(4,5- | | | |
| | dichlorothiazol-2- | | | |
| | yl)ethyl)amino)-3- | | | |
| | methyl-2H-indazol-2- | | | |
| | yl)acetamide | | | |
| Example 317 Method 9 Mixture of Stereoisomers | 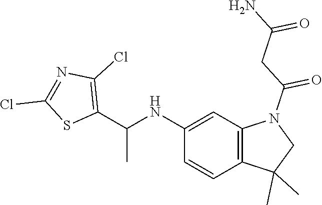 [SMILES: CC(Nc1ccc2c(c1)N(C(=O)CC(N)=O)CC2(C)C)c1sc(Cl)nc1Cl] | White solid, yield: 37% | ¹H NMR (400 MHz, DMSO-d₆) δ 7.51 (s, 1H), 7.45 (d, J = 2.2 Hz, 1H), 7.07 (s, 1H), 6.93 (d, J = 8.1 Hz, 1H), 6.42 (d, J = 5.1 Hz, 1H), 6.16 (dd, J = 8.1, 2.2 Hz, 1H), 4.66-4.52 (m, 1H), 3.80 (s, 2H), 3.33 (s, 2H), 1.49 (d, J = 6.7 Hz, 3H), 1.20 (s, 6H). | 427.05 |
| | 3-(6-((1-(2,4- | | | |
| | dichlorothiazol-5- | | | |
| | yl)ethyl)amino)-3,3- | | | |
| | dimethylindolin-1-yl)- | | | |
| | 3-oxopropanamide | | | |
| Example 320 Method 9 single unknown stereoisomer | 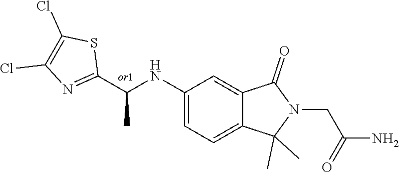 [SMILES: C[C@H](Nc1ccc2c(c1)C(=O)N(CC(N)=O)C2(C)C)c1nc(Cl)c(Cl)s1] | White solid, yield: 50.1% | ¹H NMR (400 MHz, Methanol-d₄) δ 7.33 (d, J = 8.3 Hz, 1H), 6.96 (d, J = 8.4 Hz, 1H), 6.91 (s, 1H), 4.78-4.69 (m, 1H), 4.12 (s, 2H), 1.63 (d, J = 6.7 Hz, 3H), 1.44 (s, 6H). | 413.05 CHIRALPAK IF 2 cm × 25 cm, 5 um, Mobile Phase A: MtBE Phase B: MeOH: DCM |
| | | | | Flow |
| | rel-(R)-2-(5-((1-(4,5- | | | Rate: 20 |
| | dichlorothiazol-2- | | | mL/min |
| | yl)ethyl)amino)-1,1- | | | 220 nm, |
| | dimethyl-3- | | | 250° C. |
| | oxoisoindolin-2- | | | Rt: 3.836 |
| | yl)acetamide | | | min. |
| Example 320A Method 9 single unknown stereoisomer | 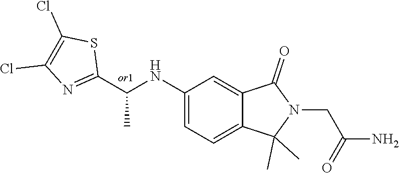 [SMILES: C[C@@H](Nc1ccc2c(c1)C(=O)N(CC(N)=O)C2(C)C)c1nc(Cl)c(Cl)s1] | White solid, yield: 50.1% | ¹H NMR (400 MHz, Methanol-d₄) δ 7.33 (d, J = 8.3 Hz, 1H), 6.95 (d, J = 8.4 Hz, 1H), 6.90 (s, 1H), 4.78-4.68 (m, 1H), 1.63 (d, J = 6.7 Hz, 3H), 1.43 (s, 6H). | 413.05 CHIRALPAK IF 2 cm × 25 cm, 5um, Mobile Phase A: MtBE Phase B: MeOH: DCM |
| | | | | Flow |
| | rel-(S)-2-(5-((1-(4,5- | | | Rate: |
| | dichlorothiazol-2- | | | 20 mL/min |
| | yl)ethyl)amino)-1,1- | | | 220 nm, |
| | dimethyl-3- | | | 25° C. |
| | oxoisoindolin-2- | | | Rt: 3.249 |
| | yl)acetamide | | | min. |
| Example 323 Method 9 single unknown stereoisomer | 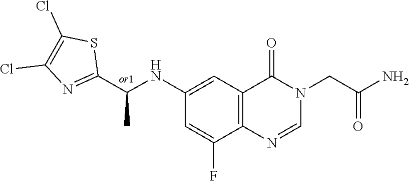 [SMILES: C[C@H](Nc1cc(F)c2ncn(CC(N)=O)c(=O)c2c1)c1nc(Cl)c(Cl)s1] | White solid, yield: 11.8% | ¹H NMR (400 MHz, DMSO-d₆) δ 8.04 (s, 1H), 7.76-7.68 (m, 1H), 7.31-7.24 (m, 2H), 7.11 (dd, J = 12.6, 2.6 Hz, 1H), 6.96 (d, J = 2.5 Hz, 1H), 4.94 (t, J = 6.6 Hz, 1H), 4.55 (s, 2H), 1.56 (d, J = 6.6 Hz, 3H). | 416.9 Note: SFC column, RT = 7.14 min |
| | rel-(R)-2-(6-((1-(4,5- | | | |
| | dichlorothiazol-2- | | | |
| | yl)ethyl)amino)-8- | | | |
| | fluoro-4- | | | |
| | oxoquinazolin-3(4H)- | | | |
| | yl)acetamide | | | |
| Example 323A Method 9 single unknown stereoisomer | 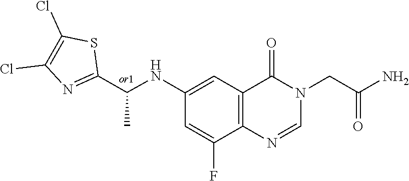 [SMILES: C[C@@H](Nc1cc(F)c2ncn(CC(N)=O)c(=O)c2c1)c1nc(Cl)c(Cl)s1] | White solid, yield: 11.8% | ¹H NMR (400 MHz, DMSO-d₆) δ 8.04 (s, 1H), 7.76-7.68 (m, 1H), 7.31-7.24 (m, 2H), 7.11 (dd, J = 12.6, 2.6 Hz, 1H), 6.96 (d, J = 2.5 Hz, 1H), 4.94 (t, J = 6.6 Hz, 1H), 4.55 (s, 2H), 1.56 (d, J = 6.6 Hz, 3H). | 416.9 Note: SFC column, RT = 8.19 min |
| | rel-(S)-2-(6-((1-(4,5- | | | |
| | dichlorothiazol-2-yl)ethyl)amino)-8- | | | |
| | fluoro-4- | | | |
| | oxoquinazolin-3(4H)- | | | |
| | yl)acetamide | | | |
| Example 327 Method 9 Mixture of Stereoisomers | 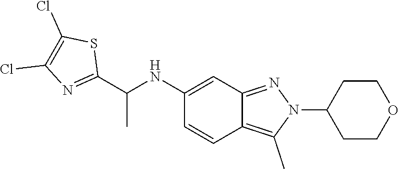 [SMILES: Cc1c2ccc(NC(C)c3nc(Cl)c(Cl)s3)cc2nn1C1CCOCC1] | White solid, yield: 54% | ¹H NMR (400 MHz, DMSO-d₆) δ 7.40 (d, J = 9.4 Hz, 1H), 6.60 (dd, J = 9.0, 2.0 Hz, 1H), 6.51 (d, J = 5.5 Hz, 1H), 6.19 (d, J = 2.1 Hz, 1H), 4.78- 4.70 (m, 1H), 4.62- 4.49 (m, 1H), 3.96 (d, J = 11.6 Hz, 2H), 3.50 (td, J = 11.9, 2.1 Hz, 2H), 2.52 (s, 3H), 2.21-2.02 | 411.2 |
| | | | (m, 2H), 1.78 (d, J = | |
| | N-(1-(4,5- | | 12.9 Hz, 2H), 1.55 (d, | |
| | dichlorothiazol-2- | | J = 6.8 Hz, 3H). | |
| | yl)ethyl)-3-methyl-2- | | | |
| | (tetrahydro-2H-pyran- | | | |
| | 4-yl)-2H-indazol-6- | | | |
| | amine | | | |
| Example 329 Method 9 Mixture of Stereoisomers | 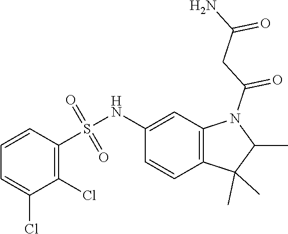 [SMILES: CC1N(C(=O)CC(N)=O)c2cc(NS(=O)(=O)c3cccc(Cl)c3Cl)ccc2C1(C)C] | White solid, yield: 4% | ¹H NMR (400 MHz, CDCl₃) δ 8.04 (m, 1H), 7.89 (s, 1H), 7.74 (d, J = 8.0 Hz, 1H), 7.38 (m, 1H), 7.04 (d, J = 8.0 Hz, 1H), 6.89 (d, J = 8.0 Hz, 1H), 4.19 (d, J = 4.0 Hz, 1H), 1.27 (s, 3H), 1.13 (m, 6H). | 470.1 |
| | 3-(6-((2,3- | | | |
| | dichlorophenyl) | | | |
| | sulfonamido)-2,3,3- | | | |
| | trimethylindolin-1-yl)- | | | |
| | 3-oxopropanamide | | | |
| Example 331 Method 9 Mixture of Stereoisomers | 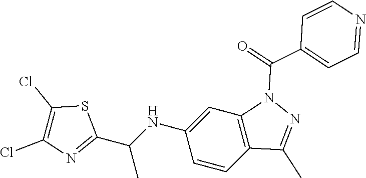 [SMILES: Cc1nn(C(=O)c2ccncc2)c2cc(NC(C)c3nc(Cl)c(Cl)s3)ccc12] | White solid, yield: 2.1% | ¹H NMR (400 MHz, DMSO-d₆) δ 8.80-8.73 (m, 2H), 7.79 (dd, J = 4.4, 1.6 Hz, 2H), 7.62 (d, J = 8.6 Hz, 1H), 7.57 (s, 1H), 7.36 (d, J = 6.0 Hz, 1H), 6.87 (dd, J = 8.6, 2.0 Hz, 1H), 4.94-4.90 (m, 1H), 1.60 (d, J = 6.8 Hz, 3H), 1.23 (s, 3H). | 432.0 |
| | (6-((1-(4,5- | | | |
| | dichlorothiazol-2- | | | |
| | yl)ethyl)amino)-3- | | | |
| | methyl-1H-indazol-1- | | | |
| | yl)(pyridin-4- | | | |
| | yl)methanone | | | |
| Example 332 Method 9 Mixture of Stereoisomers | 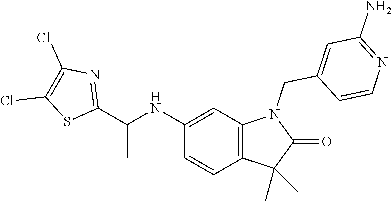 [SMILES: CC(Nc1ccc2c(c1)N(Cc1ccnc(N)c1)C(=O)C2(C)C)c1nc(Cl)c(Cl)s1] | white solid, yield: 4.9% | ¹H NMR (400 MHz, DMSO-d₆) δ 7.80 (d, J = 5.2 Hz, 1H), 7.07 (d, J = 8.0 Hz, 1H), 6.56 (d, J = 5.6 Hz, 1H), 6.31 (d, J = 5.2 Hz, 1H), 6.20 (dd, J = 8.2, 1.8 Hz, 1H), 6.17- 6.11 (m, 2H), 5.98 (s, 2H), 4.73-4.56 (m, 3H), 1.47 (d, J = 6.8 Hz, 3H), 1.28 (d, J = 4.6 Hz, 6H). | 462.1 |
| | 2,3-dichloro-N-(1,3,3- | | | |
| | trimethyl-2- | | | |
| | oxoindolin-6- | | | |
| | yl)benzenesulfonamide | | | |
| Example 334 Method 9 Mixture of Stereoisomers | 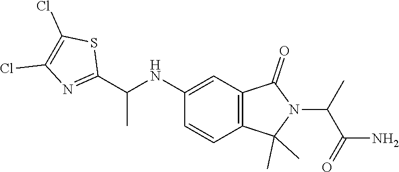 [SMILES: CC(Nc1ccc2c(c1)C(=O)N(C(C)C(N)=O)C2(C)C)c1nc(Cl)c(Cl)s1] | White solid, yield: 20.04% | ¹H NMR (400 MHz, DMSO-d₆) δ 7.34 (d, J = 8.2 Hz, 1H), 7.01-6.93 (m, 2H), 6.93-6.86 (m, 1H), 6.78 (dd, J = 5.6, 1.3 Hz, 1H), 6.72 (dd, J = 12.0, 2.2 Hz, 1H), 4.82- 4.74 (m, 1H), 4.09- 4.01 (m, 1H), 1.54 (d, 3H), 1.49 (dd, J = 7.1, 2.2 Hz, 3H), 1.42 (d, J = 2.3 Hz, 3H), 1.37 (d, J = 1.9 Hz, 3H). | 427.15 |
| | 2-(5-((1-(4,5- | | | |
| | dichlorothiazol-2- | | | |
| | yl)ethyl)amino)-1,1- | | | |
| | dimethyl-3- | | | |
| | oxoisoindolin-2- | | | |
| | yl)propanamide | | | |
| Example 335 Method 9 Mixture of Stereoisomers | 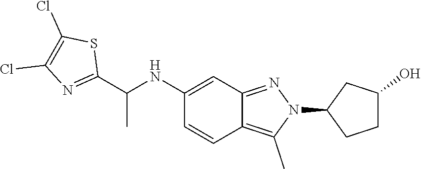 [SMILES: Cc1c2ccc(NC(C)c3nc(Cl)c(Cl)s3)cc2nn1[C@@H]1CC[C@@H](O)C1] | off- white solid, yield: 7.75% | ¹H NMR (400 MHz, Methanol-d₄) δ 7.39 (d, J = 8.8 Hz, 1H), 6.69- 6.56 (m, 1H), 6.25 (d, J = 1.6 Hz, 1H), 5.05- 4.95 (m, 1H), 4.74-4.63 (m, 1H), 4.36-4.24 (m 1H), 2.54 (s, 3H), 2.45-2.32 (m, 1H), 2.30- 2.20 (m, 1H), 2.18- 2.02 (m, 2H), 1.99-1.81 (m, 2H), 1.63 (d, J = 6.8 Hz, 3H) | 412.9 |
| | 3-(6-((1-(4,5- | | | |
| | dichlorothiazol-2- | | | |
| | yl)ethyl)amino)-3- | | | |
| | methyl-2H-indazol-2- | | | |
| | yl)cyclopentan-1-ol | | | |
| Example 336 Method 9 Mixture of Stereoisomers | 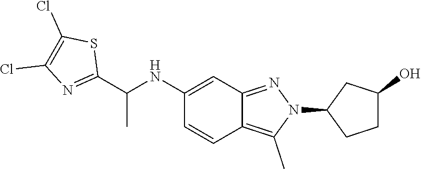 [SMILES: Cc1c2ccc(NC(C)c3nc(Cl)c(Cl)s3)cc2nn1[C@@H]1CC[C@H](O)C1] | off- white solid yield: 10.6% | ¹H NMR (400 MHz, Methanol-d₄) δ 7.39 (d, J = 8.8 Hz, 1H), 6.62 (d, J = 7.6 Hz, 1H), 6.25 (s, 1H), 5.06-4.95 (m, 1H), 4.72-4.64 (m, 1H), 4.36-4.27 (m, 1H), 2.54 (s, 3H), 2.45- 2.33 (m, 1H), 2.24-2.18 (m, 1H), 2.16-2.04 (m, 2H), 1.98-1.86 (m, 2H), 1.63 (d, J = 6.8 Hz, 3H). | 412.9 |
| | 3-(6-((1-(4,5- | | | |
| | dichlorothiazol-2- | | | |
| | yl)ethyl)amino)-3- | | | |
| | methyl-2H-indazol-2- | | | |
| | yl)cyclopentan-1-ol | | | |
| Example 337 Method 9 Mixture of Stereoisomers | 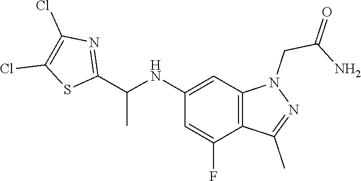 [SMILES: Cc1nn(CC(N)=O)c2cc(NC(C)c3nc(Cl)c(Cl)s3)cc(F)c12] | yellow solid, yield: 11.4% | ¹H NMR (400 MHz, DMSO-d₆) 7.38 (s, 1H), 7.19 (s, 1H), 6.90 (d, J = 6.4 Hz, 1H), 6.36-6.34 (m, 2H), 4.91-4.87 (m, 1H), 4.72 (s, 2H), 2.41 (s, 3H), 1.55 (d, J = 6.4 Hz, 3H). | 401.9 |
| | 2-(6-((1-(4,5- | | | |
| | dichlorothiazol-2- | | | |
| | yl)ethyl)amino)-4- | | | |
| | fluoro-3-methyl-1H- | | | |
| | indazol-1- | | | |
| | yl)acetamide | | | |
| Example 339 Method 9 Mixture of Stereoisomers | 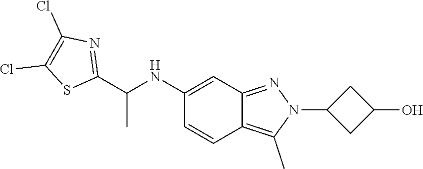 [SMILES: Cc1c2ccc(NC(C)c3nc(Cl)c(Cl)s3)cc2nn1C1CC(O)C1] | white solid, yield: 16.3% | ¹H NMR (400 MHz, CDCl₃) δ 7.38 (d, J = 8.8 Hz, 1H), 6.57 (s, 1H), 6.56-6.51 (m, 1H), 4.82- 4.74 (m, 1H), 4.63- 4.53 (m, 1H), 4.29-4.20 (m, 2H), 3.07-2.97 (m, 2H), 2.82-2.72 (m, 2H), 2.51 (s, 3H), 1.68 (d, J = 6.8 Hz, 3H). | 397.0 |
| | 3-(6-((1-(4,5- | | | |
| | dichlorothiazol-2- | | | |
| | yl)ethyl)amino)-3- | | | |
| | methyl-2H-indazol-2- | | | |
| | yl)cyclobutan-1-ol | | | |
| Example 340 Method 9 Mixture of Stereoisomers | 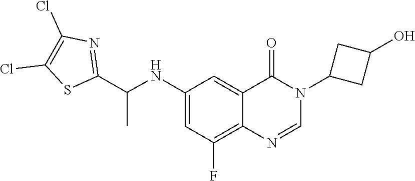 [SMILES: CC(Nc1cc(F)c2ncn(C3CC(O)C3)c(=O)c2c1)c1nc(Cl)c(Cl)s1] | white solid, yield: 66.7% | ¹HNMR (400 MHz, CD₃OD) δ 8.11 (d, J = 2.4 Hz, 1H), 7.03-7.00 (m, 2H), 4.83-4.81 (m, 1H), 4.47-4.46 (m, 1H), 4.14-4.12 (m, 1H), 2.93- 2.92 (m, 2H), 2.24- 2.23 (m, 2H), 1.64-1.63 (m, 3H). | 429.1 |
| | 6-((1-(4,5- | | | |
| | dichlorothiazol-2- | | | |
| | yl)ethyl)amino)-8- | | | |
| | fluoro-3-(3- | | | |
| | ydroxycyclobutyl) | | | |
| | quinazolin-4(3H)-one | | | |
| Example 341 Method 9 Mixture of Stereoisomers | 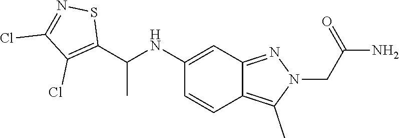 [SMILES: Cc1c2ccc(NC(C)c3snc(Cl)c3Cl)cc2nn1CC(N)=O] | White solid, yield: 15% | ¹H NMR (400 MHz, Chloroform-d) δ 7.40 (d, J = 8.9 Hz, 1H), 6.54 (d, J = 2.1 Hz, 1H), 6.46 (s, 1H), 6.32 (d, J = 2.3 Hz, 1H), 5.46 (s, 1H), 4.92 (d, J = 1.5 Hz, 2H), 4.85 (q, J = 7.4 Hz, 1H), 4.25 (s, 1H), 2.55 (s, 3H), 1.69 (d, J = 6.8 Hz, 3H). | 384.02 |
| | 2-(6-((1-(3,4- | | | |
| | dichloroisothiazol-5- | | | |
| | yl)ethyl)amino)-3- | | | |
| | methyl-2H-indazol-2- | | | |
| | yl)acetamide | | | |
| Example 341A Method 9 single unknown stereoisomer | 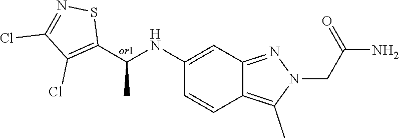 [SMILES: Cc1c2ccc(N[C@@H](C)c3snc(Cl)c3Cl)cc2nn1CC(N)=O] | White solid | ¹H NMR (400 MHz, DMSO- d₆) δ 7.51 (s, 1H), 7.40 (d, J = 8.9 Hz, 1H), 7.27-7.18 (m, 1H), 6.63-6.55 (m, 2H), 5.99 (d, J = 1.8 Hz, 1H), 4.82 (s, 2H), 4.80-4.72 (m, 1H), 2.43 (s, 3H), 1.59 (d, J = 6.7 Hz, 3H) | 384.28 CHIRALPAK ID column, 2 cm x 25 cm, 5 um, MTBE: IPA. 20 mL/min, |
| | | | | Rt = 2.268 |
| | rel-(S)-2-(6-((1-(3,4- | | | min |
| | dichloroisothiazol-5- | | | |
| | yl)ethyl)amino)-3- | | | |
| | methyl-2H-indazol-2- | | | |
| | yl)acetamide | | | |
| Example 341B Method 9 single unknown stereoisomer | 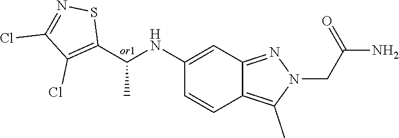 [SMILES: Cc1c2ccc(N[C@H](C)c3snc(Cl)c3Cl)cc2nn1CC(N)=O] | White solid, | ¹H NMR (400 MHz, DMSO- d₆) δ 7.51 (s, 1H), 7.40 (d, J = 8.9 Hz, 1H), 7.27-7.18 (m, 1H), 6.63-6.55 (m, 2H), 5.99 (d, J = 1.8 Hz, 1H), 4.82 (s, 2H), 4.80-4.72 (m, 1H), 2.43 (s, 3H), 1.59 (d, J = 6.7 Hz, 3H). | 384.28 CHIRALPAK ID column, 2 cm x 25 cm, 5 um MTBE: IPA. 20 mL/min, |
| | | | | Rt = 1.72 |
| | rel-(R)-2-(6-((1-(3,4- | | | min |
| | dichloroisothiazol-5- | | | |
| | yl)ethyl)amino)-3- | | | |
| | methyl-2H-indazol-2- | | | |
| | yl)acetamide | | | |
| Example 343 Method 9 Mixture of Stereoisomers | 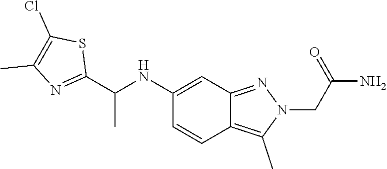 [SMILES: Cc1nc(C(C)Nc2ccc3c(C)n(CC(N)=O)nc3c2)sc1Cl] | White solid, yield: 18% | ¹H NMR (400 MHz, DMSO-d₆) δ 7.53 (s, 1H), 7.37 (d, J = 9.0 Hz, 1H), 7.26-7.21 (m, 1H), 6.59 (dd, J = 9.0, 1.9 Hz, 1H), 6.46 (d, J = 5.6 Hz, 1H), 6.07 (d, J = 2.0 Hz, 1H), 4.82 (s, 2H), 4.68 (p, J = 6.8 Hz, 1H), 2.42 (s, 3H), 2.27 (s, 3H), 1.54 (d, J = 6.8 Hz, 3H). | 364.02 |
| | 2-(6-((1-(5-chloro-4- | | | |
| | methylthiazol-2- | | | |
| | yl)ethyl)amino)-3- | | | |
| | methyl-2H-indazol-2- | | | |
| | yl)acetamide | | | |
| Example 347 Method 9 single unknown stereoisomer | 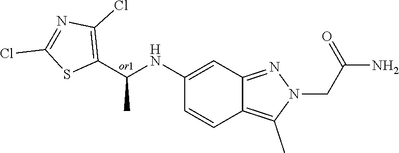 [SMILES: Cc1c2ccc(N[C@@H](C)c3sc(Cl)nc3Cl)cc2nn1CC(N)=O] | White solid, yield: 18% | ¹H NMR (400 MHz, DMSO) δ 7.62-7.52 (m, 1H), 7.41 (d, J = 8.8 Hz, 1H), 7.34-7.24 (m, 1H), 6.57 (d, J = 9.0 Hz, 1H), 6.00 (s, 1H), 4.85 (s, 1H), 4.68-4.62 (m, 1H), 2.44 (s, 2H), 1.55 (d, J = 6.7 Hz, 2H). | 393.3 Separation Conditions: Apparatus: SFC Thar prep 80; Column: CHIRALPAK IC 250 mm x |
| | | | | 20 mm, |
| | rel-(R)-2-(6-((1-(2,4- | | | 5 μm; |
| | dichlorothiazol-5- | | | Modifier: |
| | yl)ethyl)amino)-3- | | | 40% |
| | methyl-2H-indazol-2- | | | MEOH |
| | yl)acetamide | | | (NH4OH |
| | | | | 0.2%); |
| | | | | Total |
| | | | | Flow: |
| | | | | 40 g/min, |
| | | | | Rt = 5.55 |
| | | | | min) |
| Example 347A Method 9 single unknown stereoisomer | 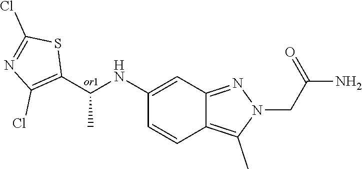 [SMILES: Cc1c2ccc(N[C@H](C)c3sc(Cl)nc3Cl)cc2nn1CC(N)=O] | white solid, yield: 3.54% | ¹H NMR (400 MHz, DMSO- d₆) δ 7.54 (s, 1H), 7.38 (d, J = 8.8 Hz, 1H), 7.25 (s, 1H), 6.54 (m, 1H), 6.46 (d, J = 4.8 Hz, 1H), 5.99 (s, 1H), 4.83 (s, 2H), 4.66-4.61 (m, 1H), 2.42 (s, 3H), 1.55 (d, J = 6.6 Hz, 3H). | 393.3 Separation Conditions: Apparatus: SFC Thar prep 80; Column: CHIRALPAK IC 250 mm × |
| | | | | 20 mm, |
| | rel-(R)-2-(6-((1-(2,4- | | | 5 μm; |
| | dichlorothiazol-5- | | | Modifier: |
| | yl)ethyl)amino)-3- | | | 40% |
| | methyl-2H-indazol-2- | | | MEOH |
| | yl)acetamide | | | (NH4OH |
| | | | | 0.2%); |
| | | | | Total |
| | | | | Flow: |
| | | | | 40 g/min, |
| | | | | Rt = 4.5 |
| | | | | min) |
| Example 348 Method 9 Mixture of Stereoisomers | 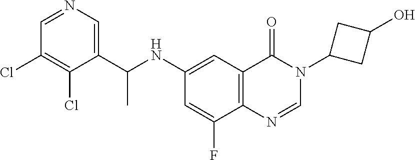 [SMILES: CC(Nc1cc(F)c2ncn(C3CC(O)C3)c(=O)c2c1)c1cncc(Cl)c1Cl] | White solid, yield: 19.2% | ¹H NMR (400 MHz, DMSO-d₆) δ 8.66 (s, 1H), 8.53 (s, 1H), 8.14 (s, 1H), 7.27 (d, J = 7.1 Hz, 1H), 6.97 (dd, J = 12.6, 2.6 Hz, 1H), 6.69 (d, J = 2.6 Hz, 1H), 5.24 (d, J = 7.0 Hz, 1H), 4.92 (t, J = 6.9 Hz, 1H), 4.46- 4.31 (m, 1H), 4.01- | 423.01 |
| | | | 3.89 (m, 1H), 2.80-2.67 | |
| | 6-((1-(4,5- | | (m, 2H), 2.20-2.09 (m, | |
| | dichloropyridin-3- | | 2H), 1.53 (d, J = 6.8 Hz, | |
| | yl)ethyl)amino)-8- | | 3H). | |
| | fluoro-3-(3- | | | |
| | hydroxycyclobutyl) | | | |
| | quinazolin-4(3H)-one | | | |
| Example 355 Method 9 Mixture of Stereoisomers | 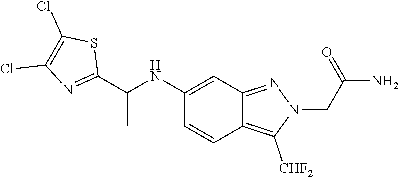 [SMILES: CC(Nc1ccc2c(C(F)F)n(CC(N)=O)nc2c1)c1nc(Cl)c(Cl)s1] | White solid, yield: 8.1% | ¹H NMR (400 MHz, Methanol-d₄) δ 7.59 (d, J = 9.2 Hz, 1H), 7.30 (t, J = 53.6 Hz, 1H), 6.83 (dd, J = 9.2, 2.0 Hz, 1H), 6.36 (d, J = 2.0 Hz, 1H), 5.12 (s, 2H), 4.73 (q, J = 6.8 Hz, 1H), 1.64 (d, J = 6.8 Hz, 3H). | 420.4 |
| | 2-(6-((1-(4,5- | | | |
| | dichlorothiazol-2- | | | |
| | yl)ethyl)amino)-3- | | | |
| | (difluoromethyl)-2H- | | | |
| | indazol-2- | | | |
| | yl)acetamide | | | |
| Example 355A Method 9 single unknown stereoisomer | 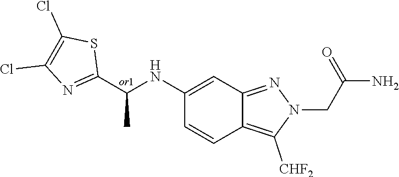 [SMILES: C[C@H](Nc1ccc2c(C(F)F)n(CC(N)=O)nc2c1)c1nc(Cl)c(Cl)s1] | white solid, yield: 21.8% | ¹H NMR (400 MHz, Methanol-d₄) δ 7.59 (d, J = 9.1 Hz, 1H), 7.31 (t, J = 53.7 Hz, 1H), 6.83 (dd, J = 9.2, 1.9 Hz, 1H), 6.36 (d, J = 1.9 Hz, 1H), 5.12 (s, 2H), 4.74 (q, J = 6.8 Hz, 1H), 1.65 (d, J = 6.8 Hz, 3H). | 420.06 Separation from 355, Note: SFC, CHIRALPAK IF 2 cm × 25 cm, 5 um |
| | | | | Modifier: |
| | rel-(S)-2-(6-((1-(4,5- | | | MtBE(0.1% |
| | dichlorothiazol-2- | | | DEA): |
| | yl)ethyl)amino)-3- | | | (MeOH: |
| | (difluoromethyl)-2H- | | | DCM = |
| | indazol-2- | | | 1:1) = 5:5 |
| | yl)acetamide | | | Rt = 3.2 |
| | | | | min |
| Example 355B Method 9 single unknown stereoisomer | 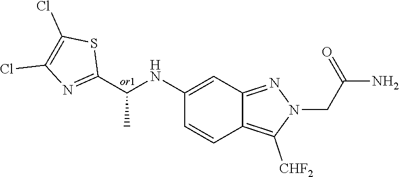 [SMILES: C[C@@H](Nc1ccc2c(C(F)F)n(CC(N)=O)nc2c1)c1nc(Cl)c(Cl)s1] | white solid, yield: 23.5% | ¹H NMR (400 MHz, Methanol-d₄) 8 7.60 (d, J = 9.1 Hz, 1H), 7.31 (t, J = 53.7 Hz, 1H), 6.91- 6.80 (m, 1H), 6.36 (d, J = 1.9 Hz, 1H), 5.12 (s, 2H), 4.74 (q, J = 6.8 Hz, 1H), 1.65 (d, J = 6.8 Hz, 3H). | 420.06 Separation from 355, Note: SFC CHIRALPAK IF 2 cm × 25 cm, 5 um |
| | | | | Modifier: |
| | rel-(R)-2-(6-((1-(4,5- | | | MtBE(0.1% |
| | dichlorothiazol-2- | | | DEA): |
| | yl)ethyl)amino)-3- | | | (MeOH: |
| | (difluoromethyl)-2H- | | | DCM = |
| | indazol-2- | | | 1:1) = 95:5 |
| | yl)acetamide | | | Rt = 2.2 |
| | | | | min |
| Example 357 Method 9 Single unknown stereoisomer | 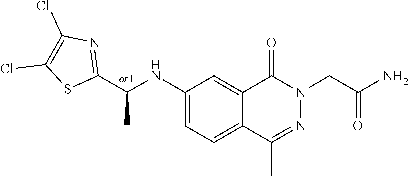 [SMILES: Cc1nn(CC(N)=O)c(=O)c2cc(N[C@@H](C)c3nc(Cl)c(Cl)s3)ccc12] | white solid, yield: 24.17% | ¹H NMR (400 MHz, DMSO-d₆) δ 7.72 (d, J = 9.2 Hz, 1H), 7.55 (d, J = 6.0 Hz, 1H), 7.45 (s, 1H), 7.25 (d, J = 6.8 Hz, 2H), 7.10 (s, 1H), 5.05- 4.97 (m, 1H), 4.57 (s, 2H), 2.41 (s, 3H), 1.59 (d, J = 6.4 Hz, 3H). | 411.8, 413.9 NOTE: Chiral- IC, 5 um, 20*250 mm, n- Hexane/ EtOH, 20 mL/min, RT = 10.7 min |
| | Rel-(S)-2-(7-((1-(4,5- | | | |
| | dichlorothiazol-2- | | | |
| | yl)ethyl)amino)-4- | | | |
| | methyl-1- | | | |
| | oxophthalazin-2(1H)- | | | |
| | yl)acetamide | | | |
| Example 358 Method 9 Single unknown stereoisomer | 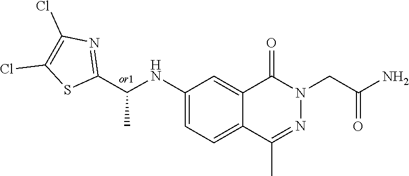 [SMILES: Cc1nn(CC(N)=O)c(=O)c2cc(N[C@H](C)c3nc(Cl)c(Cl)s3)ccc12] | white solid, yield: 25.83% | ¹H NMR (400 MHz, DMSO-d₆) δ 7.72 (d, J = 9.2 Hz, 1H), 7.55 (d, J = 6.0 Hz, 1H), 7.45 (s, 1H), 7.25 (d, J = 6.8 Hz, 2H), 7.10 (s, 1H), 5.05- 4.97 (m, 1H), 4.57 (s, 2H), 2.41 (s, 3H), 1.59 (d, J = 6.4 Hz, 3H). | 411.8, 413.9 NOTE: Chiral- IC, 5 um, 20*250 mm, n-Hexane/ EtOH, 20 mL/min, RT = 17.5 min |
| | Rel-(R)-2-(7-((1-(4,5- | | | |
| | dichlorothiazol-2- | | | |
| | yl)ethyl)amino)-4- | | | |
| | methyl-1- | | | |
| | oxophthalazin-2(1H)- | | | |
| | yl)acetamide | | | |
| Example 361 Method 9 Mixture of stereoisomers | 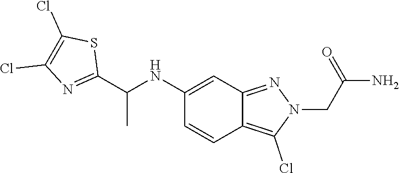 [SMILES: CC(Nc1ccc2c(Cl)n(CC(N)=O)nc2c1)c1nc(Cl)c(Cl)s1] | White solid, yield: 53.6% | ¹H NMR (400 MHz, DMSO-d₆) δ 7.60 (s, 1H), 7.34 (d, J = 9.0 Hz, 1H), 7.28 (s, 1H), 6.77 (t, J = 7.5 Hz, 2H), 6.23 (s, 1H), 4.90 (s, 2H), 4.80 (t, J = 6.6 Hz, 1H), 1.57 (d, J = 6.7 Hz, 3H). | 404.2 |
| | 2-(3-chloro-6-((1-(4,5- | | | |
| | dichlorothiazol-2- | | | |
| | yl)ethyl)amino)-2H- | | | |
| | indazol-2- | | | |
| | yl)acetamide | | | |
| Example 362 Method 9 single unknown stereoisomer | 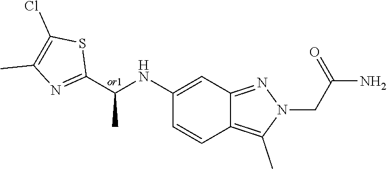 [SMILES: Cc1nc([C@H](C)Nc2ccc3c(C)n(CC(N)=O)nc3c2)sc1Cl] | White solid, yield: 35.5% | ¹H NMR (400 MHz, DMSO-d₆) δ 7.48 (s, 1H), 7.38 (d, J = 8.9 Hz, 1H), 7.22 (s, 1H), 6.59 (d, J = 9.0 Hz, 1H), 6.42 (d, J = 5.5 Hz, 1H), 6.10 (s, 1H), 4.82 (s, 2H), 4.64 (p, J = 6.1 Hz, 1H), 2.43 (s, 3H), 2.30 (s, 3H), 1.54 (d, J = 6.8 Hz, 3H). | 364.1 Note: Chiral seperation from 343, CHIRALPAK OJ, 5 um 3*250 mm Ethanol |
| | | | | (0.1% DEA) |
| | rel-(S)-2-(6-((1-(5- | | | —CO₂, |
| | chloro-4- | | | 70 mL/min, |
| | methylthiazol-2- | | | Rt = |
| | yl)ethyl)amino)-3- | | | 0.794 min |
| | methyl-2H-indazol-2- | | | |
| | yl)acetamide | | | |
| Example 363 Method 9 single unknown stereoisomer | 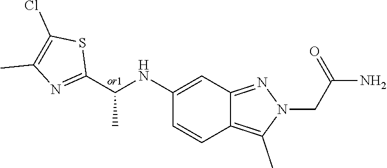 [SMILES: Cc1nc([C@@H](C)Nc2ccc3c(C)n(CC(N)=O)nc3c2)sc1Cl] | White solid, yield: 37.7% | ¹H NMR (400 MHz, DMSO-d₆) δ 7.48 (s, 1H), 7.38 (d, J = 8.9 Hz, 1H), 7.22 (s, 1H), 6.59 (d, J = 9.0 Hz, 1H), 6.42 (d, J = 5.5 Hz, 1H), 6.10 (s, 1H), 4.82 (s, 2H), 4.64 (p, J = 6.1 Hz, 1H), 2.43 (s, 3H), 2.30 (s, 3H), 1.54 (d, J = 6.8 Hz, 3H). | 364.1 Note: Chiral seperation from 343, CHIRALPAK OJ, 5 um 3*250 mm Ethanol |
| | | | | (0.1% DEA) |
| | rel-(R)-2-(6-((1-(5- | | | —CO₂, |
| | chloro-4- | | | 70 mL/min, |
| | methylthiazol-2- | | | Rt = |
| | yl)ethyl)amino)-3- | | | 0.675 min |
| | methyl-2H-indazol-2- | | | |
| | yl)acetamide | | | |
| Example 364 Method 9 Mixture of Stereoisomers | 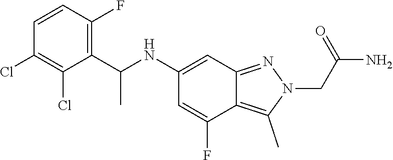 [SMILES: Cc1c2c(F)cc(NC(C)c3c(F)ccc(Cl)c3Cl)cc2nn1CC(N)=O] | white solid, yield: 8.13% | 1H NMR (400 MHz, DMSO-d₆) δ 7.60-7.52 (m, 2H), 7.25-7.19 (m, 2H), 6.44 (d, J = 8.0 Hz, 1H), 6.35 (dd, J = 13.5, 1.5 Hz, 1H), 5.73 (d, J = 1.4 Hz, 1H), 5.00 (p, J = 7.2 Hz, 1H), 4.81 (s, 2H), 2.47 (s, 3H), 1.58 (d, J = 6.9 Hz, 3H). | 413.25 |
| | 2-(6-((1-(2,3-dichloro- | | | |
| | 6- | | | |
| | fluorophenyl)ethyl) | | | |
| | amino)-4-fluoro-3- | | | |
| | methyl-2H-indazol-2- | | | |
| | yl)acetamide | | | |
| Example 365 Method 9 Mixture of Stereoisomers | 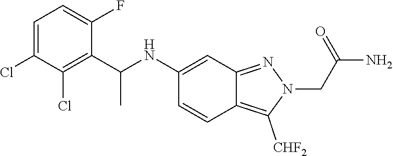 [SMILES: CC(Nc1ccc2c(C(F)F)n(CC(N)=O)nc2c1)c1c(F)ccc(Cl)c1Cl] | white solid, yield: 18.6% | ¹H NMR (400 MHz, Methanol-d₄) δ 7.40 (d, J = 9.0 Hz, 1H), 7.35- 6.98 (m, 2H), 6.90 (t, J = 9.7 Hz, 1H), 6.72-6.63 (m, 1H), 6.17 (s, 1H), 5.11 (q, J = 7.0 Hz, 1H), 4.96 (s, 2H), 1.56 (d, J = 6.9 Hz, 3H). | 431.12 |
| | 2-(6-((1-(2,3-dichloro- | | | |
| | 6- | | | |
| | fluorophenyl)ethyl) | | | |
| | amino)-3- | | | |
| | (difluoromethyl)-2H- | | | |
| | indazol-2- | | | |
| | yl)acetamide | | | |
| Example 366 Method 9 Mixture of Stereoisomers | 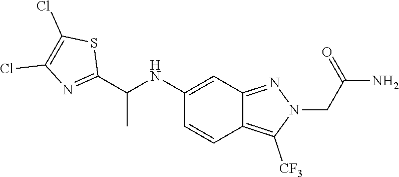 [SMILES: CC(Nc1ccc2c(C(F)(F)F)n(CC(N)=O)nc2c1)c1nc(Cl)c(Cl)s1] | White solid, yield: 42.5% | ¹H NMR (400 MHz, DMSO-d₆) δ 7.64 (s, 1H), 7.49 (s, 1H), 7.33 (s, 1H), 6.92 (s, 2H), 6.38 (s, 1H), 5.05 (s, 2H), 4.91-4.70 (m, 1H), 1.58 (d, J = 7.6 Hz, 3H). | 438.2 |
| | 2-(6-((1-(4,5- | | | |
| | dichlorothiazol-2- | | | |
| | yl)ethyl)amino)-3- | | | |
| | (trifluoromethyl)-2H- | | | |
| | indazol-2- | | | |
| | yl)acetamide | | | |
| Example 367 Method 9 single unknown stereoisomer | 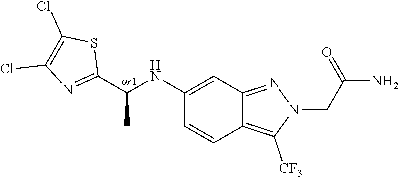 [SMILES: C[C@H](Nc1ccc2c(C(F)(F)F)n(CC(N)=O)nc2c1)c1nc(Cl)c(Cl)s1] | White solid, yield: 40.2% | ¹H NMR (400 MHz, Chloroform-d) δ 7.59 (d, J = 9.0 Hz, 1H), 6.78 (dd, J = 9.1, 2.1 Hz, 1H), 6.55 (s, 1H), 6.08 (s, 1H), 5.47 (s, 1H), 5.12 (s, 2H), 4.78 (q, J = 6.7 Hz, 1H), 1.72 (d, J = 6.8 Hz, 3H). | 438.2 Note: CHIRALPAK IG, 5 um 20*250 mm, Ethanol/ Hexane, 20 mL/min, Rt = 8.210 min |
| | rel-(S)-2-(6-((1-(4,5- | | | |
| | dichlorothiazol-2- | | | |
| | yl)ethyl)amino)-3- | | | |
| | (trifluoromethyl)-2H- | | | |
| | indazol-2- | | | |
| | yl)acetamide | | | |
| Example 368 Method 9 single unknown stereoisomer | 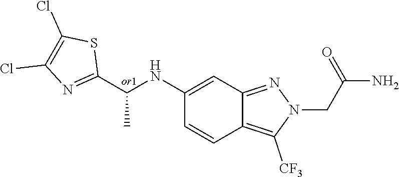 [SMILES: C[C@@H](Nc1ccc2c(C(F)(F)F)n(CC(N)=O)nc2c1)c1nc(Cl)c(Cl)s1] | White solid, yield: 39.5% | ¹H NMR (400 MHz, Chloroform-d) δ 7.59 (d, J = 9.0 Hz, 1H), 6.78 (dd, J = 9.1, 2.1 Hz, 1H), 6.55 (s, 1H), 6.08 (s, 1H), 5.47 (s, 1H), 5.12 (s, 2H), 4.78 (q, J = 6.7 Hz, 1H), 1.72 (d, J = 6.8 Hz, 3H). | 438.2 Note: CHIRALPAK IG, 5 um 20*250 mm, Ethanol/ Hexane, 20 mL/min, Rt = 6.779 min |
| | rel-(R)-2-(6-((1-(4,5- | | | |
| | dichlorothiazol-2- | | | |
| | yl)ethyl)amino)-3- | | | |
| | (trifluoromethyl)-2H- | | | |
| | indazol-2- | | | |
| | yl)acetamide | | | |
| Example 376 Method 9 Mixture of Stereoisomers | 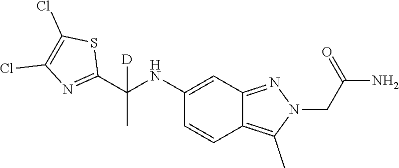 [SMILES: [2H]C(C)(Nc1ccc2c(C)n(CC(N)=O)nc2c1)c1nc(Cl)c(Cl)s1] | White solid, yield: 42% | ¹H NMR (400 MHz, DMSO-d₆) δ 7.52 (s, 1H), 7.41 (d, J = 9.0 Hz, 1H), 7.25 (s, 1H), 6.61 (dd, J = 9.0, 2.0 Hz, 1H), 6.55 (s, 1H), 6.15 (d, J = 2.0 Hz, 1H), 4.84 (s, 2H), 2.44 (s, 3H), 1.55 (s, 3H). | 385.1 |
| | 2-(6-((1-(4,5- | | | |
| | dichlorothiazol-2- | | | |
| | yl)ethyl-1-d)amino)-3- | | | |
| | methyl-2H-indazol-2- | | | |
| | yl)acetamide | | | |
| Example 377 Method 9 single unknown stereoisomer | 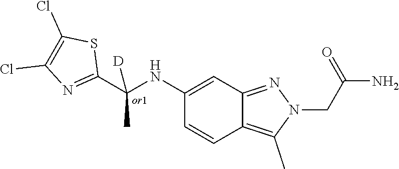 [SMILES: [2H][C@](C)(Nc1ccc2c(C)n(CC(N)=O)nc2c1)c1nc(Cl)c(Cl)s1] | White solid, yield: 39.2% | ¹H NMR (400 MHz, Chloroform-d) δ 7.40 (d, J = 8.9 Hz, 1H), 6.56 (dd, J = 8.9, 2.0 Hz, 1H), 6.51 (s, 1H), 6.45 (d, J = 2.0 Hz, 1H), 5.44 (s, 1H), 4.92 (s, 2H), 4.26 (s, 1H), 2.55 (s, 3H), 1.68 (s, 3H). | 385.1 Note: CHIRALPAK OJ, 5 um 20*250 mm, Ethanol/ Hexane, 20 mL/min, Rt = 7.245 min |
| | rel-(S)-2-(6-((1-(4,5- | | | |
| | dichlorothiazol-2- | | | |
| | yl)ethyl-1-d)amino)-3- | | | |
| | methyl-2H-indazol-2- | | | |
| | yl)acetamide | | | |
| Example 378 Method 9 single unknown stereoisomer | 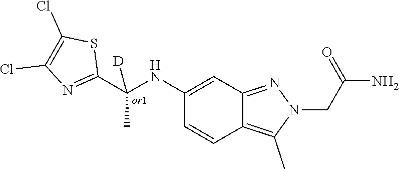 [SMILES: [2H][C@@](C)(Nc1ccc2c(C)n(CC(N)=O)nc2c1)c1nc(Cl)c(Cl)s1] | White solid, yield: 38.1% | ¹H NMR (400 MHz, Chloroform-d) δ 7.40 (d, J = 8.9 Hz, 1H), 6.56 (dd, J = 8.9, 2.0 Hz, 1H), 6.51 (s, 1H), 6.45 (d, J = 2.0 Hz, 1H), 5.44 (s, 1H), 4.92 (s, 2H), 4.26 (s, 1H), 2.55 (s, 3H), 1.68 (s, 3H). | 385.1 Note: CHIRALPAK OJ, 5 um 20*250 mm, Ethanol/ Hexane, 20 mL/min, Rt = 6.022 min |
| | rel-(R)-2-(6-((1-(4,5- | | | |
| | dichlorothiazol-2- | | | |
| | yl)ethyl-1-d)amino)-3- | | | |
| | methyl-2H-indazol-2- | | | |
| | yl)acetamide | | | |
| Example 379 Method 9 Mixture of Stereoisomers | 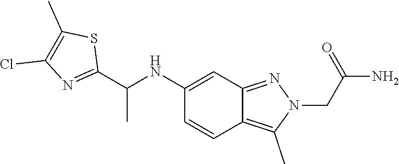 [SMILES: Cc1sc(C(C)Nc2ccc3c(C)n(CC(N)=O)nc3c2)nc1Cl] | white solid, yield: 69.9% | ¹H NMR (400 MHz, CDCl₃) δ 7.39 (d, J = 8.8 Hz, 1H), 6.71 (s, 1H), 6.58 (d, J = 8.8 Hz, 1H), 6.47 (s, 1H), 5.40 (s, 1H), 4.95 (s, 2H), 4.84- 4.76 (m, 1H), 4.36 (s, 1H), 2.56 (s, 3H), 2.31 (s, 3H), 1.68 (d, J = 6.8 Hz, 3H). | 364.1 |
| | 2-(6-((1-(4-chloro-5- | | | |
| | methylthiazol-2- | | | |
| | yl)ethyl)amino)-3- | | | |
| | methyl-2H-indazol-2- | | | |
| | yl)acetamide | | | |
| Example 380 Method 9 single unknown stereoisomer | 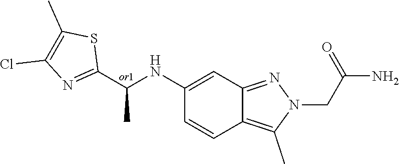 [SMILES: Cc1sc([C@H](C)Nc2ccc3c(C)n(CC(N)=O)nc3c2)nc1Cl] | white solid, yield: 35.8% | 1H NMR (400 MHz, CDCl₃) δ 7.38 (d, J = 8.9 Hz, 1H), 6.55 (dd, J = 8.9, 1.8 Hz, 1H), 6.49 (s, 1H), 6.46 (s, 1H), 5.45 (s, 1H), 4.89 (s, 2H), 4.79 (m, 1H), 4.29 (s, 1H), 2.53 (s, 3H), 2.30 (s, 3H), 1.67 (d, J = 6.7 Hz, 3H). | 364.2 Note: CHIRALPAK OJ 5 um 3*250 mm Ethanol (0.1% DEA) —CO₂— 17MIN- 220NM70 |
| | | | | mL/minute |
| | Rel-(S)-2-(6-((1-(4- | | | Rt = 0.786 |
| | chloro-5- | | | min |
| | methylthiazol-2- | | | |
| | yl)ethyl)amino)-3- | | | |
| | methyl-2H-indazol-2- | | | |
| | yl)acetamide | | | |
| Example 381 Method 9 single unknown stereoisomer | 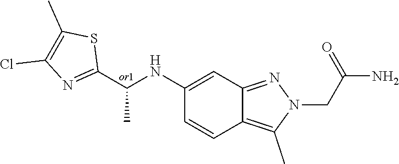 [SMILES: Cc1sc([C@@H](C)Nc2ccc3c(C)n(CC(N)=O)nc3c2)nc1Cl] | white solid, yield: 37.7% | ¹H NMR (400 MHz, CDCl₃) δ 7.38 (d, J = 8.9 Hz, 1H), 6.56 (dd, J = 8.9, 1.8 Hz, 1H), 6.51 (s, 1H), 6.46 (s, 1H), 5.45 (s, 1H), 4.90 (s, 2H), 4.79 (d, J = 6.5 Hz, 1H), 4.29 (s, 1H), 2.53 (s, 3H), 2.30 (s, 3H), 1.67 (d, J = 6.7 Hz, 3H). | 364.2 Note: CHIRALPAK OJ 5 um 3*250 mm Ethanol (0.1% DEA) —CO₂— 17MIN- 220NM70 |
| | | | | mL/minute |
| | Rel-(R)-2-(6-((1-(4- | | | Rt = |
| | chloro-5- | | | 0.668 min |
| | methylthiazol-2- | | | |
| | yl)ethyl)amino)-3- | | | |
| | methyl-2H-indazol-2- | | | |
| | yl)acetamide | | | |
| Example 389 Method 9 Mixture of Stereoisomers | 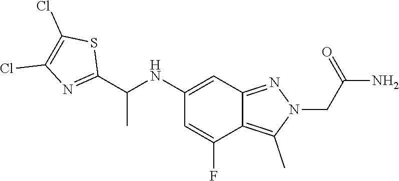 [SMILES: Cc1c2c(F)cc(NC(C)c3nc(Cl)c(Cl)s3)cc2nn1CC(N)=O] | White solid, yield: 35% | ¹H NMR (400 MHz, DMSO-d₆) δ 7.58 (s, 1H), 7.28 (s, 1H), 6.68 (d, J = 5.2 Hz, 1H), 6.33 (d, J = 12.8 Hz, 1H), 5.99 (s, 1H), 4.86 (s, 2H), 4.78-4.75 (m, 1H), 2.51 (s, 3H), 1.55 (d, J = 6.4 Hz, 3H), | 402.3 |
| | 2-(6-((1-(4,5- | | | |
| | dichlorothiazol-2- | | | |
| | yl)ethyl)amino)-4- | | | |
| | fluoro-3-methyl-2H- | | | |
| | indazol-2- | | | |
| | yl)acetamide | | | |
| Example 390 Method 9 single unknown stereoisomer | 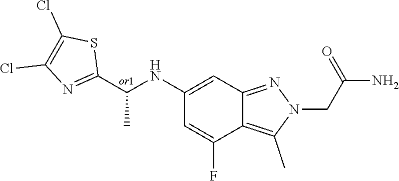 [SMILES: Cc1c2c(F)cc(N[C@H](C)c3nc(Cl)c(Cl)s3)cc2nn1CC(N)=O] | White solid | ¹H NMR (400 MHz, DMSO-d₆) δ 7.59 (s, 1H), 7.29 (s, 1H), 6.68 (d, J = 5.7 Hz, 1H), 6.34 (d, J = 13.1 Hz, 1H), 5.99 (s, 1H), 4.86 (s, 2H), 4.76 (p, J = 6.4 Hz, 1H), 2.51 (s, 3H), 1.55 (d, J = 6.7 Hz, 3H). | 402.3 Note: CHIRAL Cellulose SB, 2 cm*25 cm, 5 um, MeOH/ DCM, MtBE, 20 mL/min, Rt = 2.2 min |
| | rel-(R)-2-(6-((1-(4,5- | | | |
| | dichlorothiazol-2- | | | |
| | yl)ethyl)amino)-4- | | | |
| | fluoro-3-methyl-2H- | | | |
| | indazol-2- | | | |
| | yl)acetamide | | | |
| Example 391 Method 9 single unknown stereoisomer | 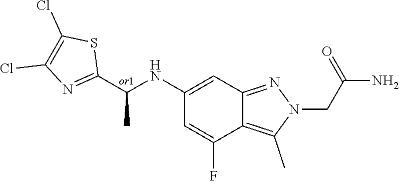 [SMILES: Cc1c2c(F)cc(N[C@@H](C)c3nc(Cl)c(Cl)s3)cc2nn1CC(N)=O] | White solid | ¹H NMR (400 MHz, DMSO-d₆) δ 7.59 (s, 1H), 7.29 (s, 1H), 6.68 (d, J = 5.7 Hz, 1H), 6.34 (d, J = 13.1 Hz, 1H), 5.99 (s, 1H), 4.86 (s, 2H), 4.76 (p, J = 6.4 Hz, 1H), 2.51 (s, 3H), 1.55 (d, J = 6.7 Hz, 3H). | 402.3 Note: CHIRAL Cellulose SB, 2 cm*25 cm, 5 um, MeOH/ DCM, MtBE, 20 mL/min, Rt = 3.03 min |
| | rel-(S)-2-(6-((1-(4,5- | | | |
| | dichlorothiazol-2- | | | |
| | yl)ethyl)amino)-4- | | | |
| | fluoro-3-methyl-2H- | | | |
| | indazol-2- | | | |
| | yl)acetamide | | | |

### Preparation of 2-[6-[(2,3-dichlorophenyl)sulfonylamino]-3,3-dimethyl-2-oxo-indolin-1-yl]acetic Acid (241)

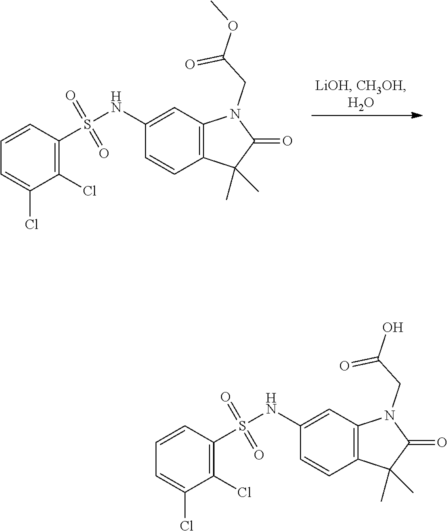 [SMILES: COC(=O)CN1C(=O)C(C)(C)c2ccc(NS(=O)(=O)c3cccc(Cl)c3Cl)cc21 ; CC1(C)C(=O)N(CC(=O)O)c2cc(NS(=O)(=O)c3cccc(Cl)c3Cl)ccc21]

To a solution of methyl 2-[6-[(2,3-dichlorophenyl)sulfonylamino]-3,3-dimethyl-2-oxo-indolin-1-yl]acetate (1.00 eq, 40 mg, 0.088 mmol) in methanol (1 mL) was added lithium hydroxide (2.00 eq, 4.2 mg, 0.175 mmol) in water (0.2 mL) and then stirred at RT for 2 h. The reaction was acidified with 1N HCl to pH ˜5 and concentrated under vacuum. The residue was purified by Prep-HPLC to afford the desired product 241 (2.8 mg, 0.0062 mmol, 7.08% yield) as a white solid. ¹H NMR (400 MHz, DMSO-d₆) δ 8.05 (dd, J=7.9, 1.6 Hz, 1H), 7.88 (dd, J=8.1, 1.5 Hz, 1H), 7.52 (t, J=8.0 Hz, 1H), 7.18 (d, J=8.0 Hz, 1H), 6.69 (dd, J=8.0, 1.9 Hz, 1H), 6.64 (d, J=1.9 Hz, 1H), 4.25 (s, 2H), 1.19 (s, 6H). Mass (m/z): 443.3 [M+H]⁺.

### Preparation of 6-((2,3-dichlorophenyl)sulfonamido)-3,3-dimethylindoline-1-carboxamide (242) Method 10

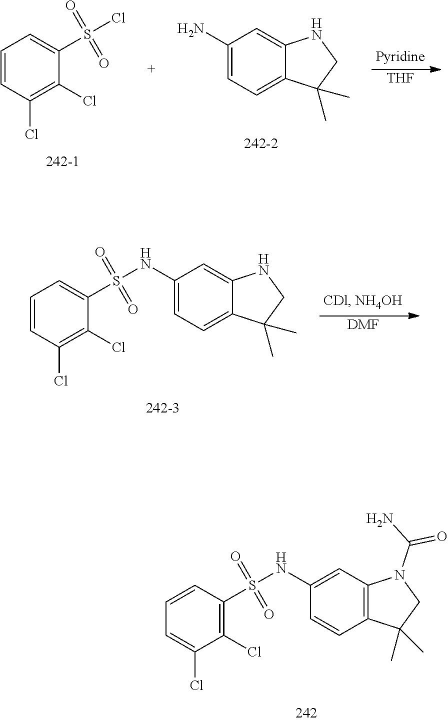

Step 1: To a solution of 2,3-dichlorobenzenesulfonyl chloride 242-1 (1.00 eq, 454 mg, 1.849 mmol) and Pyridine (3.00 eq, 438 mg, 5.548 mmol) in THF (10 mL) was added 3,3-dimethylindolin-6-amine 242-2 (1.00 eq, 300 mg, 1.85 mmol) at 25° C. The mixture was stirred at 25° C. for 12 h. Added H₂O (20 mL) and extracted with EtOAc. The combined organic layers were washed with HCl solution (1 mol/L) (25 mL×3) and dried over Na₂SO₄, filtered, and concentrated in vacuo. The crude product was purified by silica column to afford 2,3-dichloro-N-(2-oxoindolin-6-yl)benzenesulfonamide 242-3 (380 mg, 0.97 mmol, 52.6% yield) as a white solid. Mass (m/z): 371/373 [M+H]⁺.

Step 2: To a solution of 2,3-dichloro-N-(3,3-dimethylindolin-6-yl)benzenesulfonamide 242-3 (1.00 eq, 50 mg, 0.135 mmol) in DMF (10 mL) was added 1,1′-Carbonyldiimidazole (3.00 eq, 66 mg, 0.404 mmol). The reaction was stirred overnight at 25° C. Ammonium hydroxide (5.00 eq, 24 mg, 0.673 mmol) was added and the mixture was stirred 5 min at 25° C. The mixture was then extracted by EA (25 mL×3). The combined organic layers were washed with brine (15 mL×3), dried over Na₂SO₄ and concentrated to give the crude product, which was purified by prep-HPLC to give the desired product 242 (5.6 mg, 0.0134 mmol, 9.94% yield) as a white solid. Mass (m/z): 414.1/416.1 [M+H]⁺. ¹H NMR (400 MHz, DMSO-d₆) δ 10.61 (s, 1H), 7.99 (dd, J=8.0, 1.6 Hz, 1H), 7.90 (dd, J=8.0, 1.6 Hz, 1H), 7.66 (d, J=2.0 Hz, 1H), 7.52 (t, J=8.0 Hz, 1H), 6.97 (d, J=8.0 Hz, 1H), 6.59 (dd, J=8.0, 2.0 Hz, 1H), 6.24 (s, 2H), 3.57 (s, 2H), 1.18 (s, 6H).

Compounds 243, 276, 279-281, 284-285, 295-296, 299, 309, 313, 319, 326, and 333 were synthesized using a method similar to that used for Compound 242.

| Example | | Appear- | | |
|---|---|---|---|---|
| (Compound) | | ance | | |
| No. | | and | | MS |
| Method | Structure and Name | Yield | ¹H NMR Data | (m/z) |
| Example 243 Method 10 | 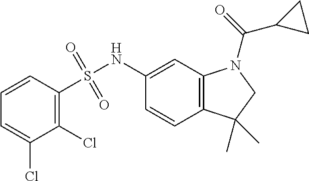 [SMILES: CC1(C)CN(C(=O)C2CC2)c2cc(NS(=O)(=O)c3cccc(Cl)c3Cl)ccc21] | white solid, yield: 38.5% | ¹H NMR (400 MHz, DMSO- d₆) δ 10.70 (s, 1H), 7.99 (dd, J = 8.0, 1.6 Hz, 1H), 7.90 (dd, J = 8.0, 1.6 Hz, 2H), 7.53 (t, J = 8.0 Hz, 1H), 7.08 (d, J = 8.0 Hz, 1H), 6.74 (dd, J = 8.0, 2.0 Hz, 1H), 3.98 (s, 2H), 1.92- 1.80 (m, 1H), 1.23 (s, 6H), 0.95-0.72 (m, 4H). | 439.0/ 441.0 |
| | 2,3-dichloro-N-(1- | | | |
| | (cyclopropanecarbonyl)- | | | |
| | 3,3-dimethylindolin-6- | | | |
| | yl)benzenesulfonamide | | | |
| Example 276 Method 10 Mixture of Stereoisomers | 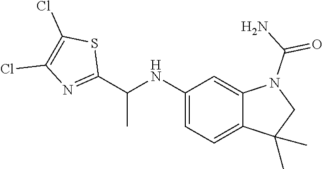 [SMILES: CC(Nc1ccc2c(c1)N(C(N)=O)CC2(C)C)c1nc(Cl)c(Cl)s1] | white solid, yield: 15.1% | ¹H NMR (400 MHz, DMSO- d₆) δ 7.30 (d, J = 2.0 Hz, 1H), 6.84 (d, J = 8.0 Hz, 1H), 6.42 (d, J = 5.6 Hz, 1H), 6.18 (s, 2H), 6.06 (dd, J = 8.0, 2.0 Hz, 1H), 4.59 (p, J = 6.8 Hz, 1H), 3.56 (s, 2H), 1.49 (d, J = 6.8 Hz, 3H), 1.19 (s, 6H). | 385.0/ 387.0 |
| | 6-((1-(4,5- | | | |
| | dichlorothiazol-2- | | | |
| | yl)ethyl)amino)-3,3- | | | |
| | dimethylindoline-1- | | | |
| | carboxamide | | | |
| Example 279 Method 10 | 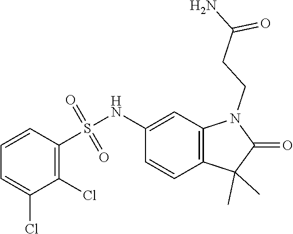 [SMILES: CC1(C)C(=O)N(CCC(N)=O)c2cc(NS(=O)(=O)c3cccc(Cl)c3Cl)ccc21] | white solid, yield: 12.3% | ¹H NMR (400 MHz, DMSO- d₆) δ 11.14 (s, 1H), 8.08 (dd, J = 8.0, 1.6 Hz, 1H), 7.85 (d, J = 8.0 Hz, 1H), 7.49 (t, J = 8.0 Hz, 1H), 7.40 (s, 1H), 7.10- 7.06 (m, 1H), 6.88 (s, 1H), 6.73 (s, 1H), 6.64 (dt, J = 4.0, 2.0 Hz, 1H), 3.73 (dd, J = 8.4, 6.4 Hz, 2H), 2.28 (t, J = 7.2 Hz, 2H), 1.15 (d, J = 4.8 Hz, 6H). | 456.2/ 458.2. |
| | 3-(6-((2,3- | | | |
| | dichlorophenyl) | | | |
| | sulfonamido)- | | | |
| | 3,3-dimethyl-2- | | | |
| | oxoindolin-1- | | | |
| | yl)propanamide | | | |
| Example 280 Method 10 | 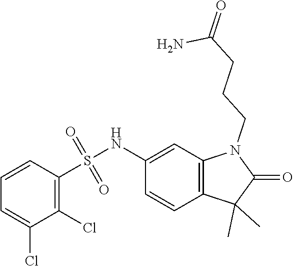 [SMILES: CC1(C)C(=O)N(CCCC(N)=O)c2cc(NS(=O)(=O)c3cccc(Cl)c3Cl)ccc21] | white solid, yield: 21.4% | ¹H NMR (400 MHz, DMSO- d₆) δ 11.03 (s, 1H), 8.13 (dd, J = 8.0, 1.6 Hz, 1H), 7.88 (dd, J = 8.0, 1.6 Hz, 1H), 7.53 (t, J = 8.0 Hz, 1H), 7.29 (s, 1H), 7.14 (d, J = 8.0 Hz, 1H), 6.81- 6.74 (m, 1H), 6.72 (d, J = 2.0 Hz, 1H), 6.66 (dd, J = 8.0, 2.0 Hz, 1H), 3.55 (t, J = 7.2 Hz, 2H), 2.02 (q, J = 8.0 Hz, 2H), 1.67 (p, J = 7.2 Hz, 2H), 1.17 (s, 6H). | 470.0/ 472.0 |
| | 4-(6-((2,3- | | | |
| | dichlorophenyl) | | | |
| | sulfonamido)- | | | |
| | 3,3-dimethyl-2- | | | |
| | oxoindolin-1- | | | |
| | yl)butanamide | | | |
| Example 281 Method 10 | 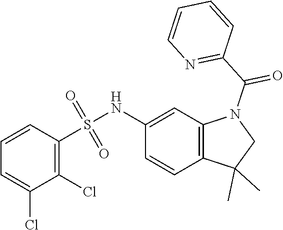 [SMILES: CC1(C)CN(C(=O)c2ccccn2)c2cc(NS(=O)(=O)c3cccc(Cl)c3Cl)ccc21] | Light- yellow solid, yield: 6.5% | ¹H NMR (400 MHz, CDCl₃) δ 8.65 (S, 1H), 8.19-8.14 (m, 1H), 7.97 (s, 3H), 7.63 (m, 2H), 7.30 (m, 1H), 7.04-7.02 (m, 1H), 6.88-6.86 (m, 1H), 3.83 (s, 2H), 1.15 (s, 6H). | 476.0 |
| | 2,3-dichloro-N-(3,3- | | | |
| | dimethyl-1- | | | |
| | picolinoylindolin-6- | | | |
| | yl)benzenesulfonamide | | | |
| Example 284 Method 10 | 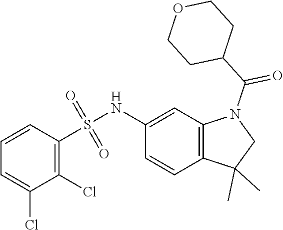 [SMILES: CC1(C)CN(C(=O)C2CCOCC2)c2cc(NS(=O)(=O)c3cccc(Cl)c3Cl)ccc21] | white solid, yield: 39.3% | ¹H NMR (400 MHz, DMSO- d₆) δ 10.68 (s, 1H), 8.01 (dd, J = 8.0, 1.5 Hz, 1H), 7.99- 7.94 (m, 1H), 7.90 (dd, J = 8.1, 1.5 Hz, 1H), 7.53 (t, J = 8.0 Hz, 1H), 7.08 (d, J = 8.1 Hz, 1H), 6.77 (dd, J = 8.1, 2.1 Hz, 1H), 4.00-3.78 (m, 4H), 3.45-3.35 (m, 2H), 2.86- 2.71 (m, 1H), 1.72-1.55 (m, 4H), 1.20 (s, 6H). | 483.08 |
| | 2,3-dichloro-N-[3,3- | | | |
| | dimethyl-1- | | | |
| | (tetrahydropyran-4- | | | |
| | carbonyl)indolin-6- | | | |
| | yl]benzenesulfonamide | | | |
| Example 285 Method 10 | 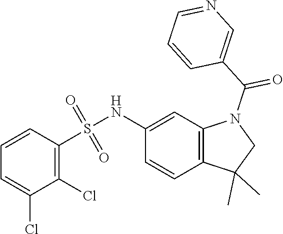 [SMILES: CC1(C)CN(C(=O)c2cccnc2)c2cc(NS(=O)(=O)c3cccc(Cl)c3Cl)ccc21] | white solid, yield: 7.1% | ¹H NMR (400 MHz, CDCl₃) δ 8.76-8.72 (m, 2H), 8.51 (m, 3H), 7.79-7.77 (m, 1H), 7.61 (m, 1H), 7.58 (m, 1H), 7.45- 7.13 (m, 1H), 6.94 (s, 1H), 3.81 (s, 2H), 1.27 (s, 6H). | 476.0 |
| | 2,3-dichloro-N-(3,3- | | | |
| | dimethyl-1- | | | |
| | nicotinoylindolin-6- | | | |
| | yl)benzenesulfonamide | | | |
| Example 295 Method 10 | 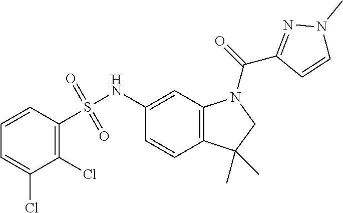 [SMILES: Cn1ccc(C(=O)N2CC(C)(C)c3ccc(NS(=O)(=O)c4cccc(Cl)c4Cl)cc32)n1] | white solid, yield: 11.7% | ¹H NMR (400 MHz, Methanol-d₄) δ 7.97 (s, 2H), 7.65-7.64 (m, 1H), 7.63 (s, 1H), 7.33-7.29 (m, 1H), 6.99- 6.97 (m, 1H), 6.81-6.67 (m, 2H), 4.20 (s, 2H), 3.87 (s, 3H), 1.16 (s, 6H). | 479.0 |
| | 2,3-dichloro-N-(3,3- | | | |
| | dimethyl-1-(1-methyl- | | | |
| | 1H-pyrazole-3- | | | |
| | carbonyl)indolin-6- | | | |
| | yl)benzenesulfonamide | | | |
| Example 296 Method 10 | 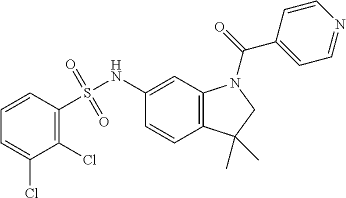 [SMILES: CC1(C)CN(C(=O)c2ccncc2)c2cc(NS(=O)(=O)c3cccc(Cl)c3Cl)ccc21] | yellow solid, yield: 10.9% | ¹H NMR (400 MHz, DMSO- d₆) δ 10.82 (s, 1H), 8.72 (d, J = 5.1 Hz, 2H), 8.18-7.88 (m, 3H), 7.62-7.53 (m, 3H), 7.15 (d, J = 8.1 Hz, 1H), 6.87 (d, J = 8.1 Hz, 1H), 3.65 (s, 2H), 1.17 (s, 6H). | 476.0 |
| | 2,3-dichloro-N-(1- | | | |
| | isonicotinoyl-3,3- | | | |
| | dimethylindolin-6- | | | |
| | yl)benzenesulfonamide | | | |
| Example 299 Method 10 | 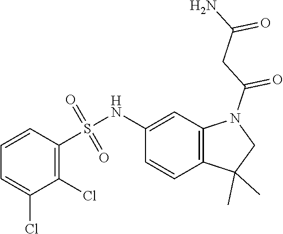 [SMILES: CC1(C)CN(C(=O)CC(N)=O)c2cc(NS(=O)(=O)c3cccc(Cl)c3Cl)ccc21] | white solid, yield: 10.6% | ¹H NMR (400 MHz, DMSO- d₆) δ 10.70 (s, 1H), 8.01 (dd, J = 8.0, 1.5 Hz, 1H), 7.92 (dd, J = 7.8, 1.6 Hz, 2H), 7.55 (t, J = 8.0 Hz, 1H), 7.51 (s, 1H), 7.09 (d, J = 8.1 Hz, 2H),6.77 (dd, J = 8.1, 2.1 Hz, 1H), 3.82 (s, 2H), 3.36 (s, 2H), 1.20 (s, 6H). | 456 |
| | 3-(6-((2,3- | | | |
| | dichlorophenyl) | | | |
| | sulfonamido)- | | | |
| | 3,3-dimethylindolin- | | | |
| | 1-yl)-3-oxopropanamide | | | |
| Example 309 Method 10 | 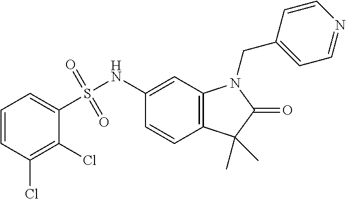 [SMILES: CC1(C)C(=O)N(Cc2ccncc2)c2cc(NS(=O)(=O)c3cccc(Cl)c3Cl)ccc21] | White solid, yield: 3.3% | ¹H NMR (400 MHz, DMSO- d₆) δ 8.50-8.47 (m, 2H), 7.85-7.79 (m, 1H), 7.74 (dd, J = 8.0, 1.6 Hz, 1H), 7.38- 7.32 (m, 1H), 7.18 (t, J = 7.4 Hz, 1H), 7.09 (dd, J = 4.6, 1.6 Hz, 2H), 6.66 (dd, J = 7.0, 5.4 Hz, 1H), 6.49-6.45 (m, 1H), 4.79 (s, 2H), 1.23 (s, 6H). | 475.9 |
| | 2,3-dichloro-N-(3,3- | | | |
| | dimethyl-2-oxo-1- | | | |
| | (pyridin-4- | | | |
| | ylmethyl)indolin-6- | | | |
| | yl)benzenesulfonamide | | | |
| Example 313 Method 10 | 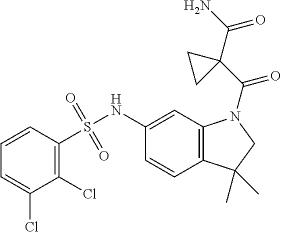 [SMILES: CC1(C)CN(C(=O)C2(C(N)=O)CC2)c2cc(NS(=O)(=O)c3cccc(Cl)c3Cl)ccc21] | White solid, yield: 46.2% | ¹H NMR (400 MHz, DMSO- d₆) δ 10.72 (br s, 1H), 8.02 (dd, J = 8.0, 1.6 Hz, 1H), 7.92 (dd, J = 8.0, 1.6 Hz, 1H), 7.89- 7.81 (m, 1H), 7.56 (t, J = 8.0 Hz, 1H), 7.17 (br s, 2H), 7.08 (d, J = 8.0 Hz, 1H), 6.78 (dd, J = 8.0, 2.0 Hz, 1H), 3.76 (s, 2H), 1.29-1.22 (m, 4H), 1.19 (s, 6H). | 482.3 |
| | 1-(6-((2,3- | | | |
| | dichlorophenyl) | | | |
| | sulfonamido)-3,3- | | | |
| | dimethylindoline-1- | | | |
| | carbonyl)cyclopropane- | | | |
| | 1-carboxamide | | | |
| Example 319 Method 10 | 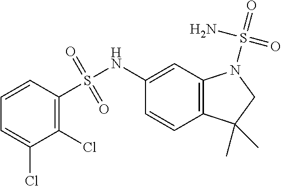 [SMILES: CC1(C)CN(S(N)(=O)=O)c2cc(NS(=O)(=O)c3cccc(Cl)c3Cl)ccc21] | white solid, yield: 7.2% | ¹H NMR (400 MHz, DMSO- d₆) δ 10.70 (s, 1H), 8.02 (dd, J = 8.0, 1.4 Hz, 1H), 7.92 (dd, J = 8.0, 1.4 Hz, 1H), 7.55 (t, J = 8.0 Hz, 1H), 7.25 (s, 2H), 7.18 (d, J = 1.9 Hz, 1H), 7.03 (d, J = 8.1 Hz, 1H), 6.64 (dd, J = 8.1, 1.9 Hz, 1H), 3.51 (s, 2H), 1.20 (s, 6H). | 450 |
| | 6-((2,3- | | | |
| | dichlorophenyl) | | | |
| | sulfonamido)-3,3- | | | |
| | dimethylindoline-1- | | | |
| | sulfonamide | | | |
| Example 326 Method 10 | 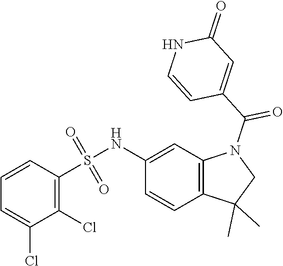 [SMILES: CC1(C)CN(C(=O)c2cc[nH]c(=O)c2)c2cc(NS(=O)(=O)c3cccc(Cl)c3Cl)ccc21] | yellow solid, yield: 0.7% | ¹H NMR (400 MHz, DMSO- d₆) δ 10.79 (s, 1H), 7.94 (dd, J = 8.0, 1.4 Hz, 3H), 7.58 (t, J = 8.0 Hz, 1H), 7.13 (d, J = 8.0 Hz, 1H), 6.83 (s, 1H), 6.49 (s, 2H), 6.25 (s, 2H), 3.66 (s, 2H), 1.17 (s, 6H). | 492 |
| | 2,3-dichloro-N-(3,3- | | | |
| | dimethyl-1-(2-oxo-1,2- | | | |
| | dihydropyridine-4- | | | |
| | carbonyl)indolin-6- | | | |
| | yl)benzenesulfonamide | | | |
| Example 333 Method 10 | 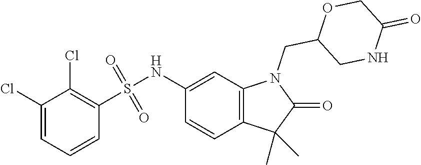 [SMILES: CC1(C)C(=O)N(CC2CNC(=O)CO2)c2cc(NS(=O)(=O)c3cccc(Cl)c3Cl)ccc21] | White solid, yield: 6.8% | ¹H NMR (400 MHz, DMSO- d₆) δ 10.82 (s, 1H), 8.09 (dd, J = 8.0, 1.4 Hz, 1H), 8.01 (d, J = 3.6 Hz, 1H), 7.93 (dd, J = 8.2, 1.4 Hz, 1H), 7.55 (t, J = 8.0 Hz, 1H), 7.19 (d, J = 8.0 Hz, 1H), 6.82 (d, J = 1.6 Hz, 1H), 6.73 (dd, J = 8.0, 1.8 Hz, 1H), 3.93 (d, J = 5.8 Hz, 2H), 3.83 (d, J = 4.2 Hz, 1H), 3.76- 3.66 (m, 2H), 3.18 (m, 1H), | 498.1 |
| | 2,3-dichloro-N-(3,3- | | 3.12-3.05 (m, 1H), 1.19 (s, | |
| | dimethyl-2-oxo-1-((5- | | 6H). | |
| | oxomorpholin-2- | | | |
| | yl)methyl)indolin-6- | | | |
| | yl)benzenesulfonamide | | | |

### Preparation of 7-((1-(2,3-dichlorophenyl)-3-hydroxypropyl)amino)isoquinolin-1(2H)-one (252) (Mixture of Stereoisomers)

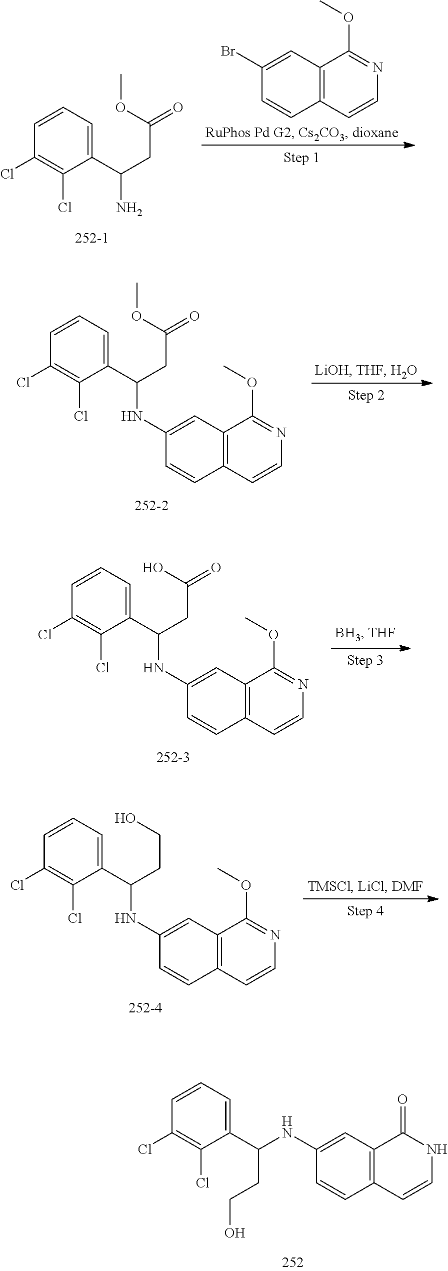 [SMILES: COC(=O)CC(N)c1cccc(Cl)c1Cl ; COc1nccc2ccc(Br)cc12 ; COC(=O)CC(Nc1ccc2ccnc(OC)c2c1)c1cccc(Cl)c1Cl ; COc1nccc2ccc(NC(CC(=O)O)c3cccc(Cl)c3Cl)cc12 ; COc1nccc2ccc(NC(CCO)c3cccc(Cl)c3Cl)cc12 ; O=c1[nH]ccc2ccc(NC(CCO)c3cccc(Cl)c3Cl)cc12]

Step 1: A mixture of 3-amino-3-(2,3-dichlorophenyl)propanoate 252-1 (400 mg, 1.6122 mmol), 7-bromo-1-methoxy-1,2-dihydroisoquinoline (387 mg, 1.61 mmol), RuPhos Pd G₂ (250 mg, 0.32 mmol) and Cs₂CO₃ (1576 mg, 4.84 mmol) in 1,4-dioxane (10 mL) was stirred under N₂ for 16 hour at 100° C. The reaction mixture was diluted with water (50 mL) after the reaction was completed, and extracted with ethyl acetate (50 mL). The organic layers were combined, dried over anhydrous Na₂SO₄ and concentrated under reduced pressure to give a crude product that was purified through column chromatography on silica gel (EA:PE=1:1) to give compound 252-2 (300 mg, 45.6% yield) as oil. MS (m/z) 405.1 [M+H]⁺.

Step 2: A mixture of 252-2 (300 mg, 0.74 mmol) and LiOH (156 mg, 3.71 mmol) in THF:H₂O (5 mL) was stirred under nitrogen at 25° C. for 2 h. The reaction mixture was extracted with EA (30 mL). Organic layers were combined, dried over anhydrous Na₂SO₄ and concentrated under reduced pressure to give a crude product that was purified through column chromatography on silica gel (MeOH:CH₂Cl₂=1:10) to give 3-(2,3-dichlorophenyl)-3-((1-methoxyisoquinolin-7-yl)amino)propanoic acid (200 mg, 68.9% yield) as oil. MS (ESI) m/z 391.1 [M+H]⁺.

Step 3: A mixture of 3-(2,3-dichlorophenyl)-3-((1-methoxyisoquinolin-7-yl)amino)propanoic acid (200 mg, 0.5128 mmol), BH₃ in THF (5 mL) was stirred for 16 hour at 50° C. The reaction was quenched with water and extracted with EA (50 mL). The organic layers were combined and concentrated under vacuum to give compound 252-3 (110 mg, 56.9% yield) as oil. MS (m/z) 377.1 [M+H]⁺.

Step 4: A mixture solution of 252-3 (110 mg, 0.28 mmol), TMSCl (61 mg, 0.5648 mmol), LiCl (24 mg, 0.5714 mmol) in DMF (3 mL) was stirred at 80° C. for 18 h. The reaction was diluted with water (20 mL) and extracted three times with EA (30 mL). The organic layers were combined, dried over anhydrous Na₂SO₄ and concentrated under reduced pressure to give a crude product that was purified by perp-HPLC (column-Gemini-C18 150×21.2 mm, 5 um; Mobile phase: ACN-H₂O (0.1% TFA), 30%-70%) to afford the desired product (31.6 mg) as a white solid. MS (m/z) 362.9 [M+H]. ¹H NMR (400 MHz, DMSO-d₆) δ 10.83 (d, J=5.0 Hz, 1H), 7.45 (dd, J=8.0, 1.6 Hz, 1H), 7.39 (dd, J=8.0, 1.5 Hz, 1H), 7.32-7.23 (m, 2H), 6.99-6.94 (m, 2H), 6.79-6.73 (m, 2H), 6.27 (d, J=7.0 Hz, 1H), 4.96 (dd, J=13.0, 7.6 Hz, 1H), 4.64 (t, J=5.0 Hz, 1H), 3.56 (ddd, J=14.8, 9.4, 5.5 Hz, 2H), 1.91-1.73 (m, 2H).

### Preparation of 7-((1-(2,3-dichlorophenyl)-2-hydroxy-2-methylpropyl)amino)isoquinolin-1(2H)-one (253) (Mixture of Stereoisomers)

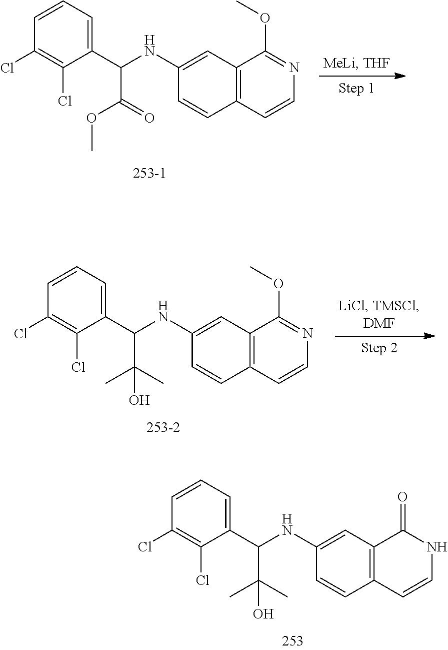 [SMILES: COC(=O)C(Nc1ccc2ccnc(OC)c2c1)c1cccc(Cl)c1Cl ; COc1nccc2ccc(NC(c3cccc(Cl)c3Cl)C(C)(C)O)cc12 ; CC(C)(O)C(Nc1ccc2cc[nH]c(=O)c2c1)c1cccc(Cl)c1Cl]

Step 1: A mixture of 253-1 (180 mg, 1.035 mmol) and MeLi (0.9 mL) in THF (5 mL) was stirred under nitrogen at −78° C. for 30 mins. The reaction was quenched with water (20 mL) and extracted with EA (30 mL), Organic layer were combined, dried over anhydrous Na₂SO₄ and concentrated under reduced pressure to give a crude product that was purified through column chromatography on silica gel (MeOH: CH₂Cl₂=1:10) to give the 253-2 (50 mg, 27.7% yield) as a yellow solid. MS (m/z) 391.1 [M+H]⁺.

Step 2: A mixture of 253-2 (50 mg, 0.13 mmol), TMSCl (28 mg, 0.26 mmol), and LiCl (11 mg, 0.26 mmol) in DMF (2 mL) was stirred at 80° C. for 18 h. The reaction was diluted with water (20 mL), and extracted three times with EA (30 mL). Organic layers were combined, dried over anhydrous Na₂SO₄ and concentrated under reduced pressure to give a crude product that was purified by perp-HPLC (column-Gemini-C18 150×21.2 mm, 5 um; Mobile phase: ACN-H₂O (0.1% FA), 40%-60%) to afford the desired product 253 (10.5 mg) as a yellow solid. ¹H NMR (400 MHz, DMSO-d₆) δ 10.79 (d, J=5.6 Hz, 1H), 7.50 (dd, J=8.0, 1.5 Hz, 1H), 7.44 (dd, J=8.0, 1.5 Hz, I H), 7.29-7.21 (m, 2H), 7.13 (d, J=2.0 Hz, 1H), 7.09 (dd, J=8.6, 2.5 Hz, 1H), 6.75 (dd, J=7.0, 5.8 Hz, 1H), 6.45 (d, J=8.8 Hz, 1H), 6.26 (d, J=6.6 Hz, 1H), 4.82 (s, 1H), 4.79 (s, 1H), 1.31 (s, 3H), 1.02 (s, 3H). MS (m/z) 377.0 [M+H]⁺.

### Preparation of 2-[6-[(2,3-dichlorophenyl)sulfonylamino]-3,3-dimethyl-2-oxo-indolin-1-yl]acetamide (255) Method 11

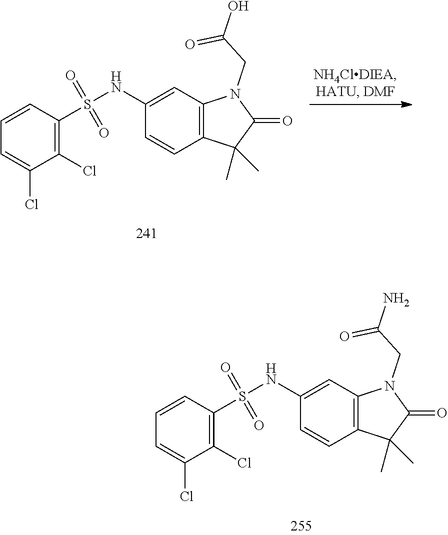 [SMILES: CC1(C)C(=O)N(CC(=O)O)c2cc(NS(=O)(=O)c3cccc(Cl)c3Cl)ccc21 ; CC1(C)C(=O)N(CC(N)=O)c2cc(NS(=O)(=O)c3cccc(Cl)c3Cl)ccc21]

A mixture of 241 (1.00 eq, 120 mg, 0.27 mmol), ammonium chloride (6.00 eq, 87 mg, 1.62 mmol), N-ethyl-N-isopropyl-propan-2-amine (10.0 eq, 350 mg, 2.71 mmol) and HATU (2.00 eq, 206 mg, 0.541 mmol) in DMF (1 mL) was stirred overnight at room temperature. The reaction was diluted with water and extracted with ethyl acetate. The organic layer was dried over sodium sulfate and concentrated under vacuum. The residue was purified by Prep-HPLC to afford 255 as a yellow solid (7.3 mg, 0.016 mmol, 5.97% yield). ¹H NMR (400 MHz, DMSO-d₆) δ 10.83 (s, 1H), 8.06 (dd, J=7.9, 1.5 Hz, 1H), 7.93 (dd, J=8.1, 1.5 Hz, 1H), 7.65 (s, 1H), 7.57 (t, J=8.1 Hz, 1H), 7.24 (s, 1H), 7.18 (d, J=8.0 Hz, 1H), 6.70 (dd, J=8.0, 2.0 Hz, 1H), 6.61 (d, J=1.9 Hz, 1H), 4.15 (s, 2H), 1.20 (s, 6H). Mass (m/z): 442.3 [M+H]⁺. Compounds 286-287, and 291 were synthesized using a method like that used for Compound 255.

| Example | | Appear- | | |
|---|---|---|---|---|
| (Compound) | | ance | | MS |
| No. | | and | | (m/z) |
| Method | Structure and Name | Yield | ¹H NMR Data | [M + H]⁺ |
| Example 286 Method 11 | 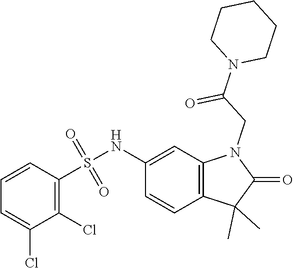 [SMILES: CC1(C)C(=O)N(CC(=O)N2CCCCC2)c2cc(NS(=O)(=O)c3cccc(Cl)c3Cl)ccc21] | white solid, yield: 43.4% | ¹H NMR (400 MHz, DMSO-d₆) δ 10.79 (s, 1H), 8.03 (dd, J = 8.0, 1.5 Hz, 1H), 7.94 (dd, J = 8.2, 1.4 Hz, 1H), 7.54 (t, J = 8.2 Hz, 1H), 7.19 (d, J = 8.0 Hz, 1H), 6.69 (dd, J = 8.0, 2.0 Hz, 1H), 6.56 (d, J = 1.8 Hz, 1H), 4.45 (s, 2H), 3.48 (s, 2H), 3.42-3.36 (m, 2H), 1.62 (s, 2H), 1.55 (s, 2H), 1.43 (s, 2H), 1.22 (s 6H) | 509.9 |
| | 2,3-dichloro-N-(3,3- | | | |
| | dimethyl-2-oxo-1-(2- | | | |
| | oxo-2-(piperidin-1- | | | |
| | yl)ethyl)indolin-6- | | | |
| | yl)benzenesulfonamide | | | |
| Example 287 Method 11 | 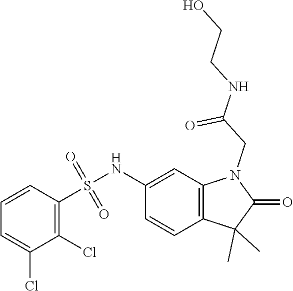 [SMILES: CC1(C)C(=O)N(CC(=O)NCCO)c2cc(NS(=O)(=O)c3cccc(Cl)c3Cl)ccc21] | white solid, yield: 15.5% | ¹H NMR (400 MHz, DMSO-d₆) δ 10.81 (s, 1H), 8.22 (t, J = 5.6 Hz, 1H), 8.05 (dd, J = 8.0, 1.4 Hz, 1H), 7.94 (dd, J = 8.2, 1.4 Hz, 1H), 7.56 (d, J = 8.2 Hz, 1H), 7.18 (d, J = 8.0 Hz, 1H), 6.70 (dd, J = 8.0, 2.0 Hz, 1H), 6.58 (d, J = 1.8 Hz, 1H), 4.19 (s, 2H), 3.46-3.41 (m, 1H), 3.15 (d, J = 5.8 Hz, 2H), 1.20 (s, 6H). | 485.9 |
| | 2-(6-((2,3- | | | |
| | dichlorophenyl) | | | |
| | sulfonamido)- | | | |
| | 3,3-dimethyl-2- | | | |
| | oxoindolin-1-yl)-N-(2- | | | |
| | hydroxyethyl) | | | |
| | acetamide | | | |
| Example 291 Method 11 | 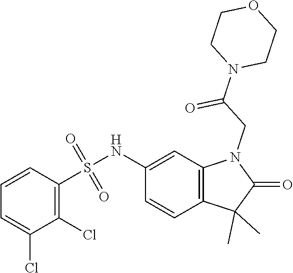 [SMILES: CC1(C)C(=O)N(CC(=O)N2CCOCC2)c2cc(NS(=O)(=O)c3cccc(Cl)c3Cl)ccc21] | white solid, yield: 24.3% | ¹H NMR (400 MHz, DMSO-d₆) δ 10.77 (s, 1H), 8.05 (dd, J = 8.0, 1.4 Hz, 1H), 7.94 (dd, J = 8.2, 1.6 Hz, 1H), 7.54 (t, J = 8.0 Hz, 1H), 7.19 (d, J = 8.0 Hz, 1H), 6.68 (dd, J = 8.0, 2.0 Hz, 1H), 6.58 (d, J = 1.8 Hz, 1H), 4.52 (s, 2H), 3.65 (d, J = 4.2 Hz, 2H), 3.58 (d, J = 4.4 Hz, 4H), 3.42 (d, J = 4.8 Hz, 2H), 1.22 (s 6H). | 511.9 |
| | 2,3-dichloro-N-(3,3- | | | |
| | dimethyl-1-(2- | | | |
| | morpholino-2- | | | |
| | oxoethyl)-2- | | | |
| | oxoindolin-6- | | | |
| | yl)benzenesulfonamide | | | |

### Preparation of 2,3-dichloro-N-(2′-oxospiro[cyclobutane-1,3′-indolin]-6′-yl)benzenesulfonamide (218) Method 13

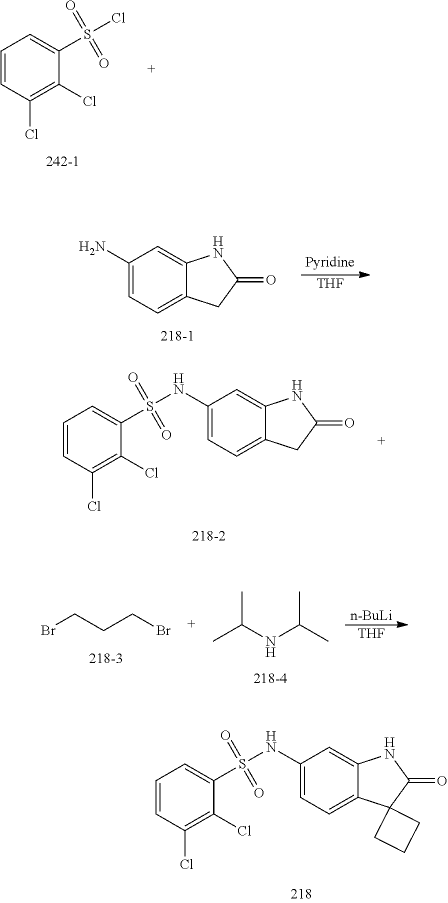

Step 1: To a solution of 2,3-dichlorobenzenesulfonyl chloride 242-1 (1.00 eq, 300 mg, 1.22 mmol) and Pyridine (3.00 eq, 0.29 mL, 3.67 mmol) in THF (10 mL) was added 6-aminoindolin-2-one 219-1 (1.00 eq, 181 mg, 1.22 mmol) at 25° C. The mixture was stirred at 25° C. for 12 h. H₂O (20 mL) was added, and the organic phase was extracted with EtOAc. The combined organic layers were washed with an HCl solution (1 mol/L) (25 mL×3) and dried over Na₂SO₄, filtered, and concentrated in vacuo. The crude product was purified by silica column to afford 218-2 (415 mg, 1.07 mmol, 87.5% yield) as a red solid. Mass (m/z): 357/359 [M+H]⁺.

Step 2: n-BuLi (4.00 eq, 0.5 mL, 1.12 mmol) was added dropwise to a stirred and cooled (−70° C.) suspension of 218-2 (1.00 eq, 100 mg, 0.28 mmol) and N-isopropylpropan-2-amine 218-4 (4 eq, 113 mg, 1.12 mmol) in dry THF (20 mL) under Ar₂. During addition the temperature was maintained below −70° C. After addition was completed, the temperature was allowed to warm to 0′C, then a solution of 1,3-dibromopropane 218-3 (1.1 eq, 62 mg, 0.308 mmol) in dry THF (10 mL) was added dropwise while maintaining a temperature of below −70° C. After addition was completed, the reaction mixture was stirred at room temperature for 18 h. The reaction mixture was concentrated to a smaller volume under reduced pressure. The residue was diluted with EtOAc (20 mL) and brine (20 mL). The biphasic mixture was then stirred vigorously. The PH was adjusted to 5 by slowly adding 4M aqueous HCL. The biphasic system was filtered through a glass filter in order to remove the solids which appeared in the biphasic system. The solids were rinsed with EtOAc, collected and dried on the air to give a first batch of 218 (1.7 mg, 0.0042 mmol, 1.51% yield) as a white solid. Mass (m/z): 397.2/399.2 [M+H]⁺. ¹H NMR (400 MHz, DMSO-d₆) δ 9.97 (s, 1H), 8.34 (s, 1H), 7.94 (dd, J=8.0, 1.6 Hz, 1H), 7.78 (d, J=8.0 Hz, 1H), 7.44 (t, J=8.0 Hz, 1H), 7.23 (d, J=8.0 Hz, 1H), 6.61-6.46 (m, 2H), 2.31 (d, J=7.2 Hz, 2H), 2.19-2.11 (m, 2H), 2.08-1.98 (d, 2H).

235 was synthesized using a similar method to that used for 218.

| Example | | Appear- | | |
|---|---|---|---|---|
| (Compound) | | ance | | |
| No. | Structure and | and | | |
| Method | Name | Yield | ¹H NMR Data | MS (m/z) |
| Example 235 Method 13 Mixture of Stereoisomers | 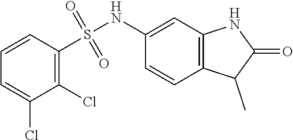 [SMILES: CC1C(=O)Nc2cc(NS(=O)(=O)c3cccc(Cl)c3Cl)ccc21] | white solid, yield: 2.7% | ¹H NMR (400 MHz, DMSO-d₆) δ 10.72 (s, 1H), 10.25 (s, 1H), 7.99 (dd, J = 8.0, 1.6 Hz, 1H), 7.92 (dd, J = 8.0, 1.6 Hz, 1H), 7.54 (t, J = 8.0 Hz, 1H), 7.07 (d, J = 8.0 Hz, 1H), 6.71-6.59 (m, | 371.0/ 373.0 |
| | 2,3-dichloro-N-(3- | | 2H), 3.27 (t, J = 7.6 | |
| | methyl-2- | | Hz, 1H), 1.22 (d, J = | |
| | oxoindolin-6- | | 7.6 Hz, 3H). | |
| | yl)benzenesulfonamide | | | |

### Preparation of 7-(1-(2,3-dichlorophenyl)-2-hydroxyethoxy)isoquinolin-1(2H)-one (228) (Mixture of Stereoisomers)

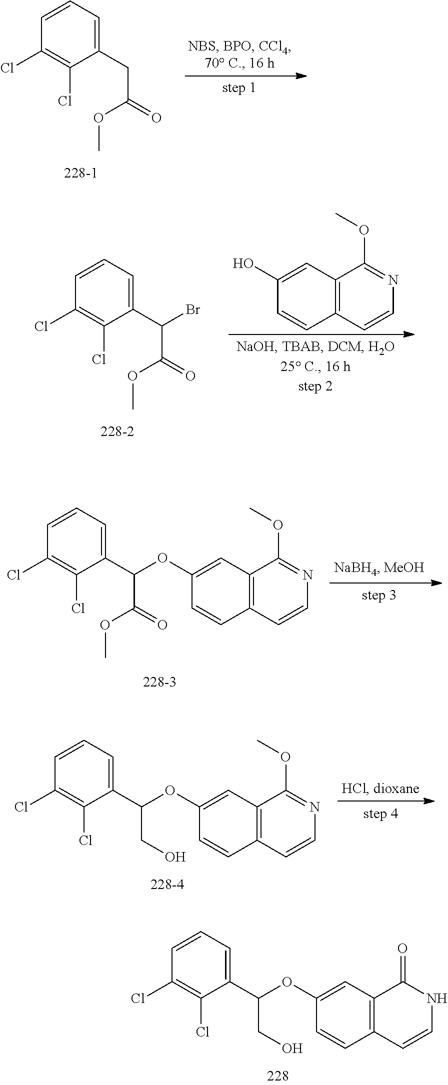 [SMILES: COC(=O)Cc1cccc(Cl)c1Cl ; COC(=O)C(Br)c1cccc(Cl)c1Cl ; COc1nccc2ccc(O)cc12 ; COC(=O)C(Oc1ccc2ccnc(OC)c2c1)c1cccc(Cl)c1Cl ; COc1nccc2ccc(OC(CO)c3cccc(Cl)c3Cl)cc12 ; O=c1[nH]ccc2ccc(OC(CO)c3cccc(Cl)c3Cl)cc12]

Step 1: To a stirred solution of 228-1 (2.00 g, 9.10 mmol), BPO (0.22 g, 0.91 mmol) in CCl₄ (20 mL) was added NBS (1.62 g, 9.10 mmol). The mixture was stirred at 70° C. for 16 hrs. LCMS showed the reaction was complete. The mixture was filtered. The filtrate was concentrated to give 228-2 (2.3 g, 84.4%) as yellow oil.

Step 2: To a solution of 228-2 (90 mg, 0.30 mmol) and 1-methoxyisoquinolin-7-ol (53 mg, 0.30 mmol), TBAB (9.7 mg, 0.03 mmol) in DCM (5 mL) was added a solution of NaOH (13 mg, 0.33 mmol) in H₂O (0.5 mL). The mixture was stirred at 25° C. for 16 hrs. The mixture was treated with H₂O (10 mL) and extracted with DCM (10 mL×3). The combined organic layers were washed with brine (10 mL), dried over Na₂SO₄, and concentrated. The residue was purified by prep-TLC (PE/EA=3:1) to give 228-3 (80 mg, 67.5%) as a yellow solid.

Step 3: To a solution of 228-3 (70 mg, 0.18 mmol) in MeOH (5 mL) was added NaBH₄ (20 mg, 0.54 mmol). The mixture was stirred at 25° C. for 4 hrs. The mixture was quenched with H₂O (20 mL) and extracted with EA (20 mL×3). The combined organic layers were washed with brine (20 mL), dried over Na₂SO₄, and concentrated. The residue was purified by prep-TLC (PE/EA=1:1) to 228-4 (48 mg, 73.6%) as a white solid.

Step 4: A solution of 228-4 (40 mg, 0.11 mmol) in dioxane (2 mL) and 6N HCl (2 mL) was stirred at 100° C. for 4 hrs. LCMS showed the reaction was completed. The mixture was concentrated and the residue was purified by prep-HPLC (sunfire 5 um 19-150 mm, Mobile Phase: MeCN—H₂O (0.1% FA), Gradient: 25-75-8 min, Flow Rate: 20 mL/min) to give 228 (26 mg, 67.4%) as a white solid. ¹H-NMR (400 MHz, DMSO-d₆): δ 11.17 (d, J=4.8 Hz, 1H), 7.63-7.54 (m, 2H), 7.46-7.40 (m, 2H), 7.37-7.30 (m, 2H), 7.01 (dd, J=6.8 Hz, 5.6 Hz, 1H), 6.47 (d, J=6.8 Hz, 1H), 5.71 (t, J=4.8 Hz, 1H), 5.37 (t, J=6.0 Hz, 1H), 3.75 (t, J=5.2 Hz, 2H). Mass (m/z): 349.9 [M+H]⁺.

### Preparation of 6-((1-(4,5-dichlorothiazol-2-yl)-3-hydroxypropyl)amino)-3,3-dimethylindolin-2-one (292) (Mixture of Stereoisomers) Method 14

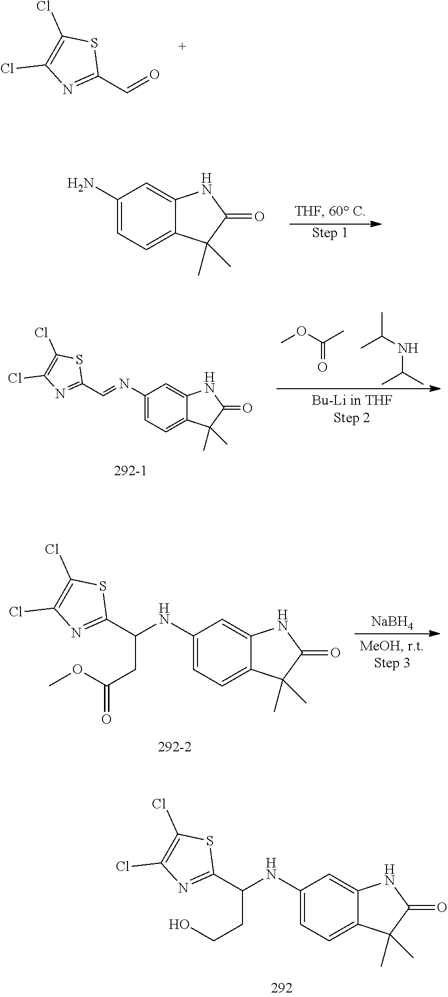

Step 1: To a solution of 4,5-dichlorothiazole-2-carbaldehyde (1.00 eq, 500 mg, 2.75 mmol) in THF (10 mL) was added 6-amino-3,3-dimethyl-indolin-2-one (1.00 eq, 484 mg, 2.75 mmol). The reaction mixture was heated to 60° C. After reaction completed, the reaction solution was concentrated in vacuo to afford 292-1 (880 mg, 2.59 mmol, 94.2% yield) as a yellow solid.

Step 2: At −40° C., n-BuLi in n-hexane (2.4 M, 10.0 eq, 7.3 mL, 17.8 mmol) was added to a solution of diisopropylamine (10.0 eq, 2.5 mL, 17.8 mmol) in distilled THF (30 mL). After stirring for 1 h, the solution was cooled to −78° C. and dry methyl acetate (10.0 eq, 1.4 mL, 17.8 mmol) was slowly added. After stirring for 1 hour, a solution of 292-1 (1.00 eq, 604 mg, 1.78 mmol) in distilled THF (15.0 mL) was added to the reaction mixture. The reaction mixture was stirred at −78° C. for 10 minutes and then the reaction was quenched by adding NH₄Cl solution at the same temperature. The reaction mixture was extracted with EA and concentrated in vacuo. The residue was subjected to flash column chromatography (EA:Hex=0-1:1) using silica gel to obtain methyl 3-(4,5-dichlorothiazol-2-yl)-3-[(3,3-dimethyl-2-oxo-indolin-6-yl)amino]propanoate (180 mg, 0.434 mmol, 24.5% yield) as a yellow solid. Mass (m/z): 414.2[M+H]⁺

Step 3: To a solution of 292-2 (1.00 eq, 60 mg, 0.145 mmol) in Methanol (15 mL) was added NaBH₄ (10.0 eq, 55 mg, 1.45 mmol). The mixture was stirred for 5 hrs at r.t. and then 20 mL of water was added. The resulting solution was extracted with EA and concentrated in vacuo. The residue was subjected to flash column chromatography (EA:Hex=0-1) using silica gel to obtain the final product 292 (25 mg, 0.065 mmol, 44.5% yield) as a white solid. Mass (m/z): 386.2[M+H]⁺⁺¹H NMR (400 MHz, Methanol-d₄) δ 10.87 (s, 1H), 7.77 (d, J=7.8 Hz, 1H), 7.33 (d, J=5.6 Hz, 1H), 7.03-6.93 (m, 2H), 5.56-5.45 (m, 2H), 4.37 (s, 2H), 2.90-2.71 (m, 2H), 1.97 (s, 6H).

Compounds 293 and 294 was synthesized using a similar method to that used in 292.

| Example | | Appear- | | |
|---|---|---|---|---|
| (Compound) | | ance | | |
| No. | | and | | MS (m/z) |
| Method | Structure and Name | Yield | ¹H NMR Data | [M + H]⁺ |
| Example 293 Method 14 Mixture of Stereoisomers | 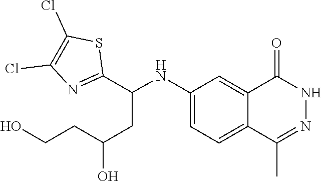 [SMILES: Cc1n[nH]c(=O)c2cc(NC(CC(O)CCO)c3nc(Cl)c(Cl)s3)ccc12] | white solid, yield: 44.5% | ¹H NMR (400 MHz, DMSO-d₆) δ 12.09 (s, 1H), 7.75-7.67 (m, 1H), 7.55-7.46 (m, 1H), 7.31-7.19 (m, 2H), 5.14-4.94 (m, 1H), 4.74 (s, 1H), 4.37 (s, 1H), 3.80 (s, 1H), 3.58-3.46 (m, 2H), 2.40 (s, 3H), 2.09-1.92 (m, 2H), 1.65-1.46 (m, 3H). | 429.3 |
| | 7-((1-(4,5- | | | |
| | dichlorothiazol-2-yl)- | | | |
| | 3,5- | | | |
| | dihydroxypentyl) | | | |
| | amino)-4- | | | |
| | methylphthalazin- | | | |
| | 1(2H)-one | | | |
| Example 294 Method 14 Mixture of Stereoisomers | 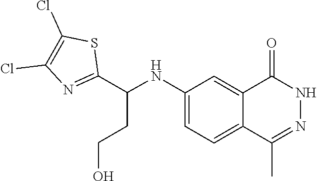 [SMILES: Cc1n[nH]c(=O)c2cc(NC(CCO)c3nc(Cl)c(Cl)s3)ccc12] | white solid, yield: 26.2% | ¹H NMR (400 MHz, DMSO-d₆) δ 12.09 (s, 1H), 7.70 (d, J = 8.7 Hz, 1H), 7.50 (d, J = 6.6 Hz, 1H), 7.36-7.16 (m, 2H), 5.05-4.92 (m, 1H), 4.75 (t, J = 5.0 Hz, 1H), 3.62-3.55 (m, 2H), 2.40 (s, 3H), 2.20- 1.96 (m, 2H). | 385.3 |

### Preparation of 6-((1-(4,5-dichlorothiazol-2-yl)ethyl)amino)-8-fluoro-3-(1H-pyrazol-5-yl)quinazolin-4(3H)-one (356-rac), rel-(S)-6-((1-(4,5-dichlorothiazol-2-yl)ethyl)amino)-8-fluoro-3-(1H-pyrazol-5-yl)quinazolin-4(3H)-one (356) and rel-(R)-6-((1-(4,5-dichlorothiazol-2-yl)ethyl)amino)-8-fluoro-3-(1H-pyrazol-5-yl)quinazolin-4(3H)-one (356A) Method 15

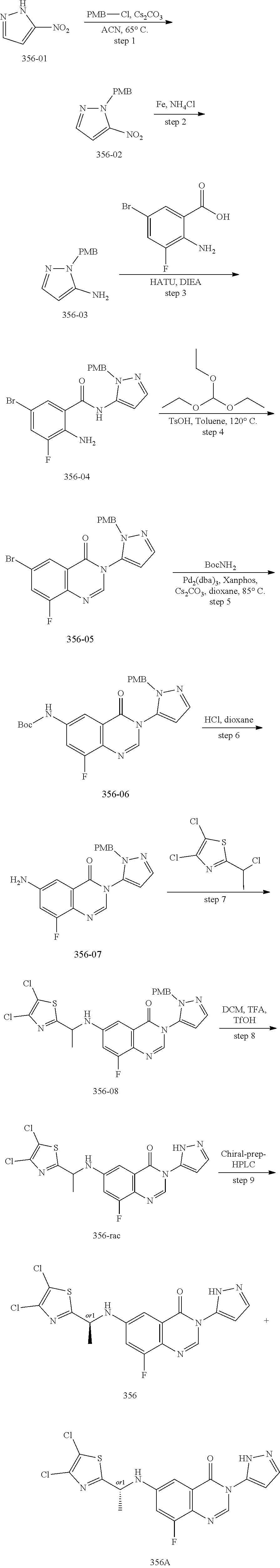

Step 1: To a solution of 356-01 (20.0 g, 0.1769 mol) in ACN (200 mL) under nitrogen was added Cesium carbonate (172.9 g, 0.53 mol) and p-Anisyl chloride (33.12 g, 0.21 mol) at 0° C. The reaction mixture was stirred at 60° C. for 16 hrs under nitrogen. The mixture was poured into water (100 mL) and extracted with EA (100 mL×3). The combined organic layers were washed with brine (50 mL), dried over Na₂SO₄, and concentrated in vacuum to give crude product 356-02 (15.0 g, 36.4%) as an off-white solid. MS (m/z): 234.1 [M+H]⁺.

Step 2: To a solution of 356-02 (12.0 g, 0.0515 mol) in THF (120 mL) and aqueous NH₄Cl solution (96 mL) was added Fe (17.26 g, 0.3090 mol) at 25° C. The mixture was stirred at 60° C. for 4 hrs. The reaction mixture was poured into water (100 mL) and extracted with EA (30 mL×3). The combined organic layers were washed with brine (30 mL), dried over Na₂SO₄ and concentrated in vacuum to give 356-03 (8.9 g, 85.1%) as a yellow solid. MS (m/z): 204.1 [M+H]⁺.

Step 3: To a solution of 356-03 (4.4 g, 0.02 mol) and 2-amino-5-bromo-3-fluorobenzoic acid (6.07 g, 0.026 mol) in DMF (40 mL) under nitrogen was added HATU (9.86 g, 0.0259 mol) and DIEA (8.37 g, 0.0648 mol) at 0° C. The reaction mixture was stirred at 25° C. for 3 hrs under nitrogen. The reaction mixture was poured into water (100 mL) and extracted with EA (50 mL×3). The combined organic layers were washed with brine, dried over Na₂SO₄ and concentrated in vacuum to give the crude product 356-04 (20.0 g, 50%) as a yellow solid. MS (m/z): 419.0 [M+H]⁺.

Step 4: To a solution of 356-04 (10.0 g, 0.0239 mol) in toluene (100 mL) was added Triethyl orthoformate (35.4 g, 0.2390 mol) and TsOH (4.11 g, 0.0239 mol) at 20° C. The reaction mixture was stirred at 120° C. for 16 hrs. The reaction mixture was poured into water (50 mL) and extracted with EA (50 mL×3). The combined organic layers were washed with brine, dried over Na₂SO₄ and concentrated in vacuum to give crude product. The crude product was purified by silica gel (PE/EA=20/1-5/1) to give 356-05 (2.9 g, 28.32%) as a brown solid. MS (m/z): 429.0 [M+H]⁺.

Step 5: To a solution of 356-04 (2.9 g, 0.0068 mol), Boc-NH₂ (0.96 g, 0.0081 mol) and Cesium carbonate (6.65 g, 0.0204 mol) in 1,4-dioxane (30 mL) under nitrogen was added Xantphos (0.39 g, 0.0007 mol) and Pd2(dba)₃ (0.31 g) at 20° C. The reaction mixture was stirred at 85° C. for 8 hrs under nitrogen. The reaction mixture was poured into water (50 mL×3) and extracted with EA (50 mL×3). The combined organic layers were washed with brine, dried over Na₂SO₄ and concentrated in vacuum to give crude product. The crude product was purified by silica gel (PE/EA=20/1-3/1) to give 356-06 (1.4 g, 44.5%) as a yellow solid. MS (m/z): 466.2 [M+H]⁺.

Step 6: A solution of 356-06 (1.3 g, 0.0028 mol) in DCM (10 mL) and 4 N HCl/Dioxane (5 mL) was stirred at 25° C. for 3 hrs. The reaction solution was concentrated and dried in vacuum to afford the crude 356-07 (1.0 g, 98.0%) as a yellow solid. MS (m/z): 366.1 [M+H]⁺.

Step 7: To a solution of 356-07 (300.0 mg, 0.8211 mmol) in DMF/H₂O=1/1 (3 mL) was added 4,5-dichloro-2-(1-chloroethyl)-1,3-thiazole (355.6 mg, 1.6422 mmol), Calcium carbonate (410.9 mg, 4.1055 mmol) and potassium iodide (68.2 mg, 0.4105 mmol) at 20° C. The reaction mixture was stirred at 100° C. for 16 hrs. The mixture was poured into water (20 mL) and extracted with EA (20 mL×3). The combined organic layers were washed with brine (15 mL), dried over Na₂SO₄ and concentrated in vacuum to give crude product 356-08 (150 mg, 33.5%) as a yellow solid. MS (m/z): 545.1 [M+H]⁺.

Step 8: A solution of 356-08 (150.0 mg, 0.2750 mmol) in DCM:TFA:TfOH=3:1:0.5 (4.5 mL) was stirred at 30° C. for 16 hrs. The mixture was poured into water (20 mL) and extracted with EA (20 mL×3). The combined organic layers were washed with brine (15 mL), dried over Na₂SO₄, and concentrated in vacuum to give crude product. The crude was purified by Prep-HPLC to give product 356-rac (47 mg, 40.2%) as a yellow solid. MS (m/z): 425.0 [M+H]⁺. The 356-rac (40 mg) was purified by prep-Chiral-HPLC (Prep-Chiral HPLC conditions: Chromatographic columns: IG; Mobile Phase: Hex (0.1% TFA)-EtOH; Gradient: HEX75(0.1% TFA): EtOH25-30 min-500VL) to give 356 (13.20 mg, 33.0% yield, RT=16.628 min, single unknown stereoisomer) and 356A (10.02 mg, 25.05% yield, RT=23.211 min, single unknown stereoisomer) as off-white solid. 356-rac: ¹H NMR (400 MHz, DMSO-d₆) δ 13.12 (s, 1H), 8.32 (s, 1H), 7.90 (s, 1H), 7.50-7.26 (m, 1H), 7.26-7.00 (m, 2H), 6.64 (s, 1H), 4.98 (m, 1H), 1.58 (d, J=6.1 Hz, 3H); 356: ¹H NMR (400 MHz, DMSO-d₆) δ 13.12 (s, 1H), 8.32 (s, 1H), 7.90 (s, 1H), 7.50-7.26 (m, 1H), 7.26-7.00 (m, 2H), 6.64 (s, 1H), 4.98 (m, 2H), 1.58 (d, J=6.1 Hz, 3H). MS (m/z): 425.2 [M+H]⁺.

356A: ¹H NMR (400 MHz, DMSO-d₆) 13.12 (s, 1H), 8.32 (s, 1H), 7.90 (s, 1H), 7.50-7.26 (m, 1H), 7.26-7.00 (m, 2H), 6.64 (s, 1H), 4.98 (m, 1H), 1.58 (d, J=6.1 Hz, 3H). MS (m/z): 425.2 [M+H]⁺.

Compounds 359 and 369-375 was synthesized using a similar method to that used in 356.

| Example | | Appear- | | |
|---|---|---|---|---|
| (Compound) | | ance | | |
| No. | | and | | MS (m/z) |
| Method | Structure and Name | Yield | ¹H NMR Data | [M + H]⁺ |
| Example 359 Method 15 Mixture of Stereoisomers | 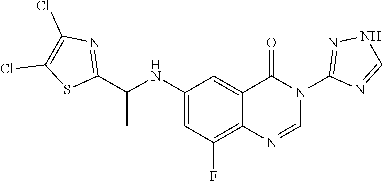 [SMILES: CC(Nc1cc(F)c2ncn(-c3nc[nH]n3)c(=O)c2c1)c1nc(Cl)c(Cl)s1] | white solid, yield: 23.45% | ¹H NMR (400 MHz, DMSO-d₆) δ 8.63 (s, 1H), 8.14 (s, 1H), 7.42 (d, J = 6.4 Hz, 1H), 7.16 (dd, J = 12.4, 2.0 Hz, 1H), 7.07 (d, J = 2.0 Hz, 1H), 5.05-4.96 (m, 1H), 1.57 (d, J = 6.8 Hz, 3H). | 425.9 |
| | 6-((1-(4,5- | | | |
| | dichlorothiazol-2- | | | |
| | yl)ethyl)amino)-8- | | | |
| | fluoro-3-(1H-1,2,4- | | | |
| | triazol-3- | | | |
| | yl)quinazolin-4(3H)- | | | |
| | one | | | |
| Example 369 Method 15 Mixture of Stereoisomers | 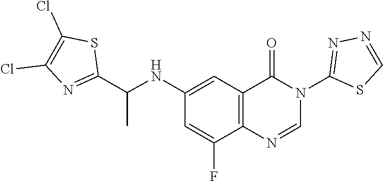 [SMILES: CC(Nc1cc(F)c2ncn(-c3nncs3)c(=O)c2c1)c1nc(Cl)c(Cl)s1] | white solid, yield: 1.39% | ¹HNMR (400 MHz, CDCl₃) δ 9.38 (s, 1H), 9.17 (s, 1H), 7.28 (d, J = 2.0 Hz, 1H), 6.93 (dd, J = 11.2, 2.8 Hz, 1H), 4.86 (m, 1H), 1.74 (d, J = 6.8 Hz, 3H). | 442.8, 444.8 |
| | 6-((1-(4,5- | | | |
| | dichlorothiazol-2- | | | |
| | yl)ethyl)amino)-8- | | | |
| | fluoro-3-(1,3,4- | | | |
| | thiadiazol-2- | | | |
| | yl)quinazolin-4(3H)- | | | |
| | one | | | |
| Example 370 Method 15 Mixture of Stereoisomers | 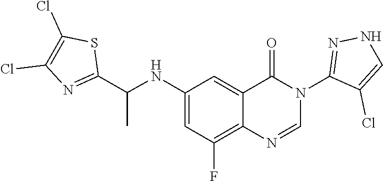 [SMILES: CC(Nc1cc(F)c2ncn(-c3n[nH]cc3Cl)c(=O)c2c1)c1nc(Cl)c(Cl)s1] | White solid, yield: 21% | ¹H NMR (400 MHz, Chloroform-d) δ 7.97 (s, 1H), 7.71 (s, 1H), 7.27 (s, 1H), 6.87 (dd, J = 11.4, 2.7 Hz, 1H), 4.84 (m, 1H), 1.71 (d, J = 6.8 Hz, 3H). | 459.2 |
| | 3-(4-chloro-1H- | | | |
| | pyrazol-3-yl)-6-((1- | | | |
| | (4,5-dichlorothiazol-2- | | | |
| | yl)ethyl)amino)-8- | | | |
| | fluoroquinazolin- | | | |
| | 4(3H)-one | | | |
| Example 371 Method 15 Mixture of Stereoisomers | 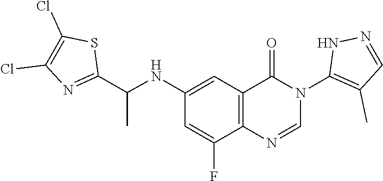 [SMILES: Cc1cn[nH]c1-n1cnc2c(F)cc(NC(C)c3nc(Cl)c(Cl)s3)cc2c1=O] | White solid, yield: 11.09% | ¹H NMR (400 MHz, DMSO-d₆) δ 12.92 (s, 1H), 8.02 (s, 1H), 7.70 (s, 1H), 7.35 (d, J = 6.7 Hz, 1H), 7.15 (dd, J = 12.6, 2.6 Hz, 1H), 7.09 (d, J = 2.6 Hz, 1H), 5.00 (p, J = 6.7 Hz, 1H), 1.88 (s, 3H), 1.58 (d, J = 6.7 Hz, 3H). | 439.29 |
| | 6-((1-(4,5- | | | |
| | dichlorothiazol-2- | | | |
| | yl)ethyl)amino)-8- | | | |
| | fluoro-3-(4-methyl- | | | |
| | 1H-pyrazol-5- | | | |
| | yl)quinazolin-4(3H)- | | | |
| | one | | | |
| Example 372 Method 15 Mixture of Stereoisomers | 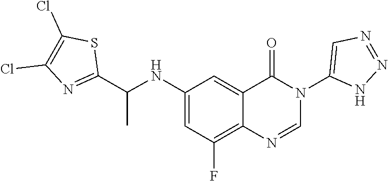 [SMILES: CC(Nc1cc(F)c2ncn(-c3cnn[nH]3)c(=O)c2c1)c1nc(Cl)c(Cl)s1] | white solid, yield: 51.2% | ¹HNMR (400 MHz, CDCl₃) δ 8.77 (s, 1H), 8.45 (s, 1H), 7.26 (m, 2H), 6.96-6.80 (m, 1H), 4.86 (m, 1H), 1.73 (d, J = 6.8 Hz, 3H). | 426.0, 428.1 |
| | 6-((1-(4,5- | | | |
| | dichlorothiazol-2- | | | |
| | yl)ethyl)amino)-8- | | | |
| | fluoro-3-(1H-1,2,3- | | | |
| | triazol-5- | | | |
| | yl)quinazolin-4(3H)- | | | |
| | one | | | |
| Example 373 Method 15 Mixture of Stereoisomers | 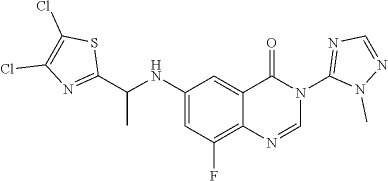 [SMILES: CC(Nc1cc(F)c2ncn(-c3ncnn3C)c(=O)c2c1)c1nc(Cl)c(Cl)s1] | white solid., yield: 1.21% | ¹H NMR (400 MHz, CDCl₃) δ 7.98 (s, 2H), 7.26 (m, 1H), 7.23 (d, J = 2.0 Hz, 1H), 6.93- 6.87 (m, 1H), 4.91-4.80 (m, 1H), 3.84 (s, 3H), 1.72 (d, J = 6.8 Hz, 1H). | 440.0, 442.0 |
| | 6-((1-(4,5- | | | |
| | dichlorothiazol-2- | | | |
| | yl)ethyl)amino)-8- | | | |
| | fluoro-3-(1-methyl- | | | |
| | 1H-1,2,4-triazol-5- | | | |
| | yl)quinazolin-4(3H)- | | | |
| | one | | | |
| Example 374 Method 15 Mixture of Stereoisomers | 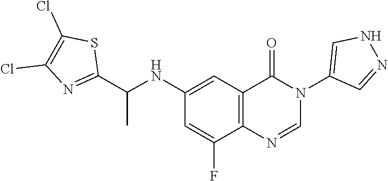 [SMILES: CC(Nc1cc(F)c2ncn(-c3cn[nH]c3)c(=O)c2c1)c1nc(Cl)c(Cl)s1] | off- white solid, yield: 20.00% | ¹HNMR (400 MHz, Methanol-d₄) δ 8.16 (s, 1H), 8.00 (s, 2H), 7.11 (d, J = 2.6 Hz, 1H), 7.05 (m, 1H), 4.85-4.83 (m, 1H), 1.66 (d, J = 6.4 Hz, 3H). | 425.0, 427.0 |
| | rel-(S)-6-((1-(4,5- | | | |
| | dichlorothiazol-2- | | | |
| | yl)ethyl)amino)-8- | | | |
| | fluoro-3-(1H-pyrazol- | | | |
| | 4-yl)quinazolin- | | | |
| | 4(3H)-one | | | |
| Example 375 Method 15 Mixture of Stereoisomers | 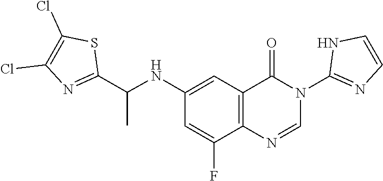 [SMILES: CC(Nc1cc(F)c2ncn(-c3ncc[nH]3)c(=O)c2c1)c1nc(Cl)c(Cl)s1] | white solid, yield: 4.09% | ¹H NMR (400 MHz, CDCl₃) δ 11.43 (br, 1H), 9.21 (s, 1H), 7.25-7.24 (m, 2H), 7.05 (s, 2H), 6.89 (dd, J = 11.4, 2.6 Hz, 1H), 4.90-4.81 (m, 1H), 1.73 (d, J = 6.7 Hz, 3H). | 425.0, 427.0 |
| | 6-((1-(4,5- | | | |
| | dichlorothiazol-2- | | | |
| | yl)ethyl)amino)-8- | | | |
| | fluoro-3-(1H- | | | |
| | imidazol-2- | | | |
| | yl)quinazolin-4(3H)- | | | |
| | one | | | |

### Preparation of 2-(6-((2-(4,5-dichlorothiazol-2-yl)propan-2-yl)amino)-3-methyl-2H-indazol-2-yl)acetamide (360)

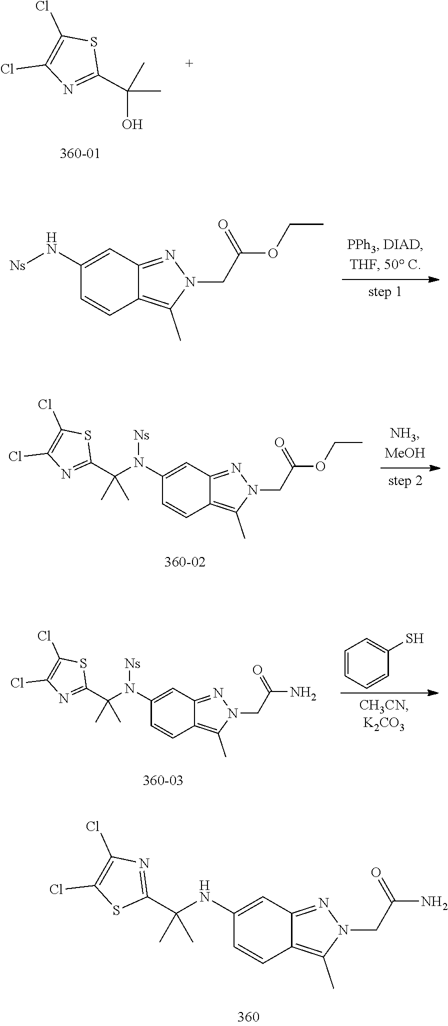

Step 1: To a solution of 360-01 (200.0 mg, 0.943 mmol), ethyl 2-{3-methyl-6-[(4-nitro-1-sulfonylphenyl) amino]indazol-2-yl}acetate (395.5 mg, 0.943 mmol) and PPh₃ (371.0 mg, 1.4145 mmol) in THF (5 mL) was added DIAD (286.0 mg, 1.4145 mmol) at 0° C. under N₂ atmosphere. The mixture was stirred at 50° C. for 16 hours. The mixture was poured into water (20 mL) and extracted with EA (10 mL×3). The combined organic layers were washed with brine (30 mL), dried over anhydrous Na₂SO₄ and concentrated to give the crude product. The crude product was purified by prep-TLC (PE/EA=1/1) to give the product 360-02 (120.0 mg, 20.78%) as a yellow solid. MS (m/z): 612.1, 614.1 [M+H]⁺.

Step 2: A mixture of 360-02 (100.0 mg, 0.163 mmol) and NH₃ in MOH (2 mL, 7 mol/L) was stirred at 25° C. for 4 hours. The mixture was concentrated to give a crude product. The crude product was purified by prep-HPLC to give the product 360-03 (11.0 mg, 11.55%) as a white solid. MS (m/z): 583.1, 585.1 [M+H]⁺.

Step 3: To a solution of 360-03 (9 mg, 0.015 mmol) in CH₃CN (8 ml) was added benzenethiol (3.3 mg, 0.03 mmol), K₂CO₃ (8.5 mg, 0.06 mmol). The mixture was stirred at 25° C. for 2 hours, was added water (30 ml) and extracted with EtOAc (1 mL×3). The solution was washed with water (2×5 ml) and saturated brine solution (30 ml). The organics were then separated and dried (MgSO4) before concentration to dryness. The crude product was purified by prep-HPLC to give the desired product as a white solid (2 mg), yield: 30.92%. ¹H NMR (400 MHz, DMSO-d₆) δ 7.54 (m, 1H), 7.39 (dd, J=8.8, 2.0 Hz, 1H), 7.25 (m, 1H), 6.57 (dd, J=9.0, 2.1 Hz, 1H), 6.38 (s, 1H), 5.82 (s, 1H), 4.83 (H, 2H), 2.42 (s, 3H), 1.65 (s, 6H). Mass (m/z): 398.31 [M+H]⁺.

Compounds 1A-15A were prepared and characterized as follows.

| Example | | Appear- | | |
|---|---|---|---|---|
| (Compound) | | ance | | |
| No. | | and | | MS (m/z) |
| Method | Structure and Name | Yield | ¹H NMR Data | [M + H]⁺ |
| Example 1A Method 2 | 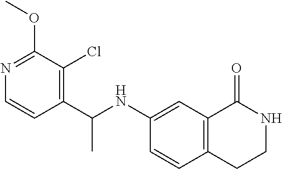 [SMILES: COc1nccc(C(C)Nc2ccc3c(c2)C(=O)NCC3)c1Cl] | White solid, yield: 8.1% | ¹H NMR (400 MHz, DMSO-d₆) δ 8.12 (d, J = 2.4 Hz, 1H), 7.87 (d, J = 2.4 Hz, 1H), 7.71 (s, 1H), 7.05 (s, 1H), 6.95 (d, J = 8.0 Hz, 1H), 6.66 (d, J = 8.0 Hz, 1H), 6.22 (s, 1H), 4.56 (d, J = 6.8 Hz, 1H), 3.89 (s, 3H), 3.25 (td, J = 6.4, 2.8 Hz, 2H), 2.67 (t, J = 6.4 Hz, | 331.11 |
| | 6-(5-(pyridazin-3- | | 2H), 1.41 (d, J = 6.8 Hz, | |
| | ylamino)pyridin-3- | | 3H). | |
| | yl)benzo[d]oxazol- | | | |
| | 2(3H)-one | | | |
| Example 2A Method 1 | 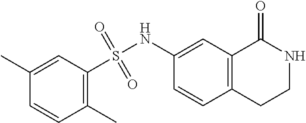 [SMILES: Cc1ccc(C)c(S(=O)(=O)Nc2ccc3c(c2)C(=O)NCC3)c1] | White solid, yield: 15.2% | ¹H NMR (400 MHz, DMSO-d₆) δ 10.41 (s, 1H), 7.89 (t, J = 2.8 Hz, 1H), 7.70 (d, J = 2.0 Hz, 1H), 7.57 (d, J = 2.0 Hz, 1H), 7.30 (dd, J = 7.6, 1.6 Hz, 1H), 7.24 (d, J = 7.6 Hz, 1H), 7.20-7.11 (m, | 330.1 |
| | 2,5-dimethyl-N-(1- | | 2H), 3.28 (td, J = 6.4, 2.8 | |
| | oxo-1,2,3,4- | | Hz, 2H), 2.75 (t, J = 6.4 | |
| | tetrahydroisoquinolin- | | Hz, 2H), 2.52 (s, 3H), | |
| | 7-yl) | | 2.29 (s, 3H). | |
| | benzenesulfonamide | | | |
| Example 3A Method 1 | 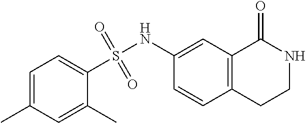 [SMILES: Cc1ccc(S(=O)(=O)Nc2ccc3c(c2)C(=O)NCC3)c(C)c1] | White solid, yield: 15.5% | ¹H NMR (400 MHz, DMSO-d₆) δ 10.39 (s, 1H), 7.88 (d, J = 2.8 Hz, 1H), 7.74 (d, J = 8.0 Hz, 1H), 7.57 (d, J = 2.4 Hz, 1H), 7.18-7.12 (m, 4H), 3.29 (td, J = 2.8, 6.4 Hz, 2H), 2.75 (t, J = 6.4 Hz, | 330.8 |
| | 2,4-dimethyl-N-(1- | | 2H), 2.54 (s, 3H), 2.28 (s, | |
| | oxo-1,2,3,4- | | 3H). | |
| | tetrahydroisoquinolin- | | | |
| | 7-yl) | | | |
| | benzenesulfonamide | | | |
| Example 4A Method 1 | 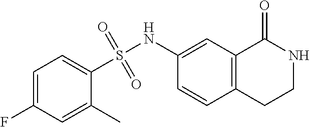 [SMILES: Cc1cc(F)ccc1S(=O)(=O)Nc1ccc2c(c1)C(=O)NCC2] | White solid, yield: 26.9% | ¹H NMR (400 MHz, DMSO-d₆) δ 10.52 (s, 1H), 7.91-7.87 (m, 2H), 7.56 (d, J = 1.6 Hz, 1H), 7.27 (dd, J = 9.6, 2.4 Hz, 1H), 7.21-7.14 (m, 3H), 3.31-3.22 (td, J = 2.8, 6.4 Hz, 2H), 2.76 (t, J = | 335.2 |
| | 4-fluoro-2-methyl-N- | | 6.8 Hz, 2H), 2.59 (s, 3H). | |
| | (1-oxo-1,2,3,4- | | | |
| | tetrahydroisoquinolin- | | | |
| | 7-yl) | | | |
| | benzenesulfonamide | | | |
| Example 5A Method 1 | 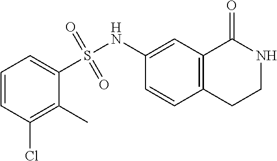 [SMILES: Cc1c(Cl)cccc1S(=O)(=O)Nc1ccc2c(c1)C(=O)NCC2] | White solid, yield: 31.4% | ¹H NMR (400 MHz, DMSO-d₆) δ 10.71 (s, 1H), 7.90 (t, J = 2.8 Hz, 1H), 7.87 (dd, J = 8.0, 1.2 Hz, 1H), 7.71 (dd, J = 8.0, 1.2 Hz, 1H), 7.56 (d, J = 1.2 Hz, 1H), 7.38 (t, J = 8.0 Hz, 1H), 7.16 (d, J = 1.6 Hz, 2H), 3.29 (td, J = 6.8, 2.8 Hz, 2H), 2.77 (t, | 351.1 |
| | 3-chloro-2-methyl-N- | | J = 6.8 Hz, 2H), 2.64 (s, | |
| | (1-oxo-1,2,3,4- | | 3H). | |
| | tetrahydroisoquinolin- | | | |
| | 7-yl) | | | |
| | benzenesulfonamide | | | |
| Example 6A Method 1 | 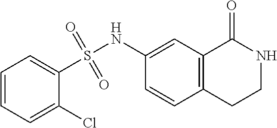 [SMILES: O=C1NCCc2ccc(NS(=O)(=O)c3ccccc3Cl)cc21] | White solid, yield: 46.1% | ¹H NMR (400 MHz, DMSO-d₆) δ 10.67 (s, 1H), 8.01 (dd, J = 7.2, 1.2 Hz, 1H), 7.90 (t, J = 2.8 Hz, 1H), 7.64-7.59 (m, 3H), 7.53 (ddd, J = 8.4, 6.4, 2.8 Hz, 1H), 7.22 (dd, J = 8.0, 2.4 Hz, 1H), | 337.1 |
| | 2-chloro-N-(1-oxo- | | 7.15 (d, J = 8.4 Hz, 1H), | |
| | 1,2,3,4- | | 3.29 (td, J = 6.4, 2.8 Hz, | |
| | tetrahydroisoquinolin- | | 2H), 2.75 (t, J = 6.4 Hz, | |
| | 7-yl) | | 2H). | |
| | benzenesulfonamide | | | |
| Example 7A Method 1 | 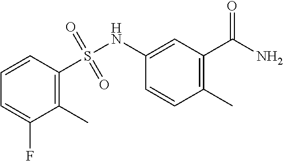 [SMILES: Cc1ccc(NS(=O)(=O)c2cccc(F)c2C)cc1C(N)=O] | White solid, yield: 35.4% | ¹H NMR (400 MHz, DMSO-d₆) δ 10.52 (br s, 1H), 7.71 (d, J = 7.2 Hz, 1H), 7.63 (s, 1H), 7.45- 7.42 (m, 2H), 7.34 (s, 1H), 7.06-6.99 (m, 3H), 2.48 (d, J = 4.0 Hz, 3H), 2.20 (s, 3H). | 323.0 |
| | 5-((3-fluoro-2- | | | |
| | methylphenyl) | | | |
| | sulfonamido)-2- | | | |
| | methylbenzamide | | | |
| Example 8A Method 1 | 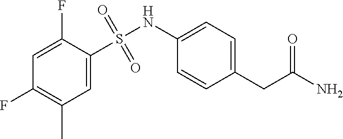 [SMILES: Cc1cc(S(=O)(=O)Nc2ccc(CC(N)=O)cc2)c(F)cc1F] | Light yellow solid, yield: 30.9% | ¹H NMR (400 MHz, DMSO-d₆) δ 10.47 (br s, 1H), 7.77 (t, J = 8.2 Hz, 1H), 7.42 (d, J = 10.0 Hz, 1H), 7.37 (br s, 1H), 7.11 (d, J = 8.6 Hz, 2H), 7.02 (d, J = 8.6 Hz, 2H), 6.80 (br s, 1H), 3.25 (s, 2H), 2.22 (s, 3H). | 341.3 |
| | 2,4-difluoro-5-methyl- | | | |
| | N-(1-oxo-1,2,3,4- | | | |
| | tetrahydroisoquinolin- | | | |
| | 7-yl) | | | |
| | benzenesulfonamide | | | |
| Example 9A Method 1 | 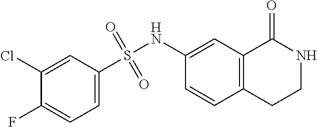 [SMILES: O=C1NCCc2ccc(NS(=O)(=O)c3ccc(F)c(Cl)c3)cc21] | White solid, yield: 13.3% | ¹H NMR (400 MHz, DMSO-d₆) δ 10.47 (s, 1H), 7.96 (s, 1H), 7.92 (dd, J = 6.8, 2.4 Hz, 1H), 7.78-7.70 (m, 1H), 7.63 (t, J = 8.8 Hz, 1H), 7.58 (s, 1H), 7.22 (d, J = 1.6 Hz, 2H), 3.34-3.26 (m, | 354.7 |
| | 3-chloro-4-fluoro-N- | | 2H), 2.80 (t, J = 6.6 Hz, | |
| | (1-oxo-1,2,3,4- | | 2H). | |
| | tetrahydroisoquinolin- | | | |
| | 7-yl) | | | |
| | benzenesulfonamide | | | |
| Example 10A Method 1 | 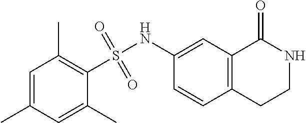 [SMILES: Cc1cc(C)c(S(=O)(=O)Nc2ccc3c(c2)C(=O)NCC3)c(C)c1] | White solid, yield: 30.0% | ¹H NMR (400 MHz, DMSO-d₆) δ 10.27 (s, 1H), 7.89 (d, J = 2.8 Hz, 1H), 7.54 (d, J = 2.4 Hz, 1H), 7.15 (d, J = 8.2 Hz, 1H), 7.08 (dd, J = 8.1, 2.4 Hz, 1H), 6.99 (s, 2H), 3.31-3.23 (m, | 345.1 |
| | 2,4,6-trimethyl-N-(1- | | 2H), 2.75 (t, J = 6.6 Hz, | |
| | oxo-1,2,3,4- | | 2H), 2.57 (s, 6H), 2.20 | |
| | tetrahydroisoquinolin- | | (s, 3H). | |
| | 7-yl) | | | |
| | benzenesulfonamide | | | |
| Example 11A Method 1 | 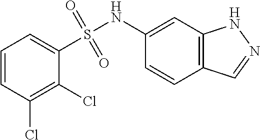 [SMILES: O=S(=O)(Nc1ccc2cn[nH]c2c1)c1cccc(Cl)c1Cl] | White solid, yield: 25.4% | ¹H NMR (400 MHz, DMSO-d₆) δ 12.87 (s, 1H), 10.90 (s, 1H), 8.02 (dd, J = 8.0, 1.6 Hz, 1H), 7.94 (s, 1H), 7.90 (dd, J = 8.0, 1.6 Hz, 1H), 7.62 (d, J = 8.6 Hz, 1H), 7.52 (t, J = 8.0 Hz, 1H), 7.25- 7.20 (m, 1H), 6.92 (dd, | 342.1 |
| | 2,3-dichloro-N-(1H- | | J = 8.6, 2.0 Hz, 1H). | |
| | indazol-6-yl) | | | |
| | benzenesulfonamide | | | |
| | (2H)-one | | | |
| Example 12A Method 2 | 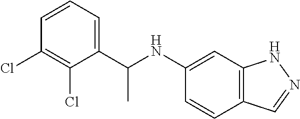 [SMILES: CC(Nc1ccc2cn[nH]c2c1)c1cccc(Cl)c1Cl] | White solid, yield: 9.5% | ¹H NMR (400 MHz, DMSO-d₆) δ 12.25 (br s, 1H), 7.72-7.67 (m, 1H), 7.50 (dd, J = 8.0, 1.6 Hz, 1H), 7.43 (dd, J = 8.0, 1.6 Hz, 1H), 7.38 (d, J = 8.8 Hz, 1H), 7.29 (t, J = 8.0 Hz, 1H), 6.69 | 306.1 |
| | N-(1-(2,3- | | (d, J = 6.4 Hz, 1H), 6.60 | |
| | dichlorophenyl)ethyl)- | | (dd, J = 8.8, 2.0 Hz, 1H), | |
| | 1H-indazol-6-amine | | 5.94 (s, 1H), 4.85-4.78 | |
| | | | (m, 1H), 1.46 (d, J = 6.8 | |
| | | | Hz, 3H). | |
| Example 13A Method 2 | 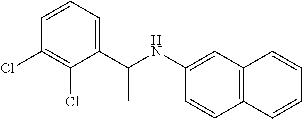 [SMILES: CC(Nc1ccc2ccccc2c1)c1cccc(Cl)c1Cl] | White solid, yield: 3.5% | ¹H NMR (400 MHz, DMSO-d₆) δ 7.60 (d, J = 8.8 Hz, 2H), 7.48 (td, J = 8.0, 1.2 Hz, 2H), 7.38 (d, J = 8.0 Hz, 1H), 7.28 (t, J = 8.0 Hz, 1H), 7.23 (t, J = 7.4 Hz, 1H), 7.09 (dd, J = 11.2, 4.0 Hz, | 316.2 |
| | N-(1-(2,3- | | 1H), 7.02 (dd, J = 8.8, | |
| | dichlorophenyl)ethyl) | | 2.0 Hz, 1H), 6.78 (d, J = | |
| | naphthalen-2-amine | | 7.2 Hz, 1H), 6.34 (d, J = | |
| | | | 2.0 Hz, 1H), 4.94 (p, J = | |
| | | | 6.8 Hz, 1H), 1.49 (d, J = | |
| | | | 6.8 Hz, 3H). | |
| Example 14A Method 1 | 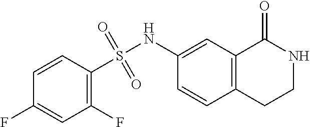 [SMILES: O=C1NCCc2ccc(NS(=O)(=O)c3ccc(F)cc3F)cc21] | White solid, yield: 45.5% | ¹H NMR (400 MHz, DMSO-d₆) δ 10.74 (s, 1H), 7.94 (t, J = 2.8 Hz, 1H), 7.87 (td, J = 8.6, 6.2 Hz, 1H), 7.60 (d, J = 2.0 Hz, 1H), 7.54 (ddd, J = 11.2, 9.0, 2.4 Hz, 1H), 7.31-7.16 (m, 3H), 3.30 | 338.3 |
| | 2,4-difluoro-N-(1- | | (td, J = 6.6, 2.8 Hz, 2H), | |
| | oxo-1,2,3,4- | | 2.79 (t, J = 6.6 Hz, 2H). | |
| | tetrahydroisoquinolin- | | | |
| | 7-yl) | | | |
| | benzenesulfonamide | | | |
| Example 15A Method 1 | 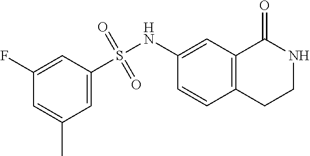 [SMILES: Cc1cc(F)cc(S(=O)(=O)Nc2ccc3c(c2)C(=O)NCC3)c1] | White solid, yield: 10% | ¹H NMR (400 MHz, Methanol-d₄) δ 7.54 (d, J = 2.3 Hz, 1H), 7.39- 7.29 (m, 2H), 7.21-7.17 (m, 2H), 7.11 (d, J = 8.2 Hz, 1H), 7.06 (d, J = 9.5 Hz, 1H), 3.35 (t, J = 6.7 Hz, 2H), 2.81 (t, J = 6.7 Hz, 2H), 2.27 (s, 3H). | 335.3 |
| | 3-fluoro-5-methyl-N- | | | |
| | (1-oxo-1,2,3,4- | | | |
| | tetrahydroisoquinolin- | | | |
| | 7-yl) | | | |
| | benzenesulfonamide | | | |

### Example II. SARM1 (50-724) Enzymatic Assay

An enzymatic assay was performed in a 384-well plate using Dulbecco's phosphate-buffered saline (PBS) as the reaction buffer. Purified SARM1 (50-724) with a final concentration of 2 nM was pre-incubated with a test compound at 1% DMSO final assay concentration for 15 min at room temperature. The reaction was initiated by adding a mixture of 200 μM nicotinamide mononucleotide (NMN) as activator and 100 μM NAD⁺ as substrate. After 1 h of incubation at room temperature, the reaction was terminated with 10 times volume of 70% acetonitrile, then centrifuged at 3800 rpm for 10 min. The samples were analyzed using LC-MS/MS after diluted to a proper concentration by 10 mM ammonium acetate (pH 9.75).

SARM1 inhibitory activity of Compounds 1-391 and Compounds 1A to 15A is summarized in Table 3. In Table 3, activity is provided as follows: A=IC₅₀≤100 nM; B=100 nM<IC₅₀≤500 nM; C=500 nM<IC₅₀≤1000 nM; D=IC₅₀>1000 nM.

TABLE 3

| Cmpd No. | IC₅₀ | Cmpd No. | IC₅₀ | Cmpd No. | IC₅₀ |
|---|---|---|---|---|---|
| 1 | B | 2 | A | 3 | B |
| 4 | D | 5 | D | 6 | B |
| 7 | D | 8 | D | 9 | D |
| 10 | D | 11 | A | 12 | D |
| 13 | A | 14 | C | 15 | A |
| 16 | B | 17 | B | 18 | C |
| 19 | B | 20 | A | 21 | B |
| 22 | A | 23 | B | 24 | D |
| 25 | C | 26 | A | 27 | D |
| 28 | D | 29 | B | 30 | B |
| 31 | B | 32 | B | 33 | B |
| 34 | D | 35 | D | 36 | B |
| 37 | A | 38 | D | 39 | A |
| 40 | A | 41 | A | 42 | A |
| 43 | B | 44 | B | 45 | A |
| 46 | D | 47 | D | 48 | D |
| 49 | D | 50 | B | 51 | D |
| 52 | B | 53 | B | 54 | D |
| 55 | A | 56 | A | 57 | B |
| 58 | A | 59 | B | 60 | D |
| 61 | D | 62 | A | 63 | B |
| 64 | B | 65 | A | 66 | D |
| 67 | B | 68 | A | 69 | A |
| 70 | A | 71 | B | 72 | A |
| 73 | A | 74 | B | 75 | A |
| 76 | B | 77 | B | 78 | A |
| 79 | B | 80 | B | 81 | A |
| 82 | A | 83 | B | 84 | A |
| 85 | B | 86 | B | 87 | B |
| 88 | A | 89 | B | 90 | B |
| 91 | A | 92 | B | 93 | C |
| 94 | D | 95 | C | 96 | D |
| 97 | C | 98 | D | 99 | D |
| 100 | B | 101 | B | 102 | B |
| 103 | A | 104 | C | 105 | B |
| 106 | D | 107 | B | 108 | B |
| 109 | B | 110 | B | 111 | A |
| 112 | A | 113 | C | 114 | B |
| 115 | D | 116 | D | 117 | B |
| 118 | A | 119 | C | 120 | D |
| 121 | C | 122 | C | 123 | D |
| 124 | D | 125 | C | 126 | B |
| 127 | D | 128 | A | 129 | C |
| 130 | B | 131 | B | 132 | A |
| 133 | B | 134 | D | 135 | A |
| 136 | A | 137 | A | 138 | A |
| 139 | B | 140 | C | 141 | D |
| 142 | C | 143 | A | 144 | B |
| 145 | C | 146 | A | 147 | B |
| 148 | D | 149 | B | 150 | D |
| 151 | B | 152 | A | 152A | C |
| 153 | A | 154 | B | 155 | B |
| 156 | A | 157 | B | 158 | D |
| 159 | C | 160 | C | 161 | D |
| 162 | D | 163 | D | 164 | D |
| 165 | D | 166 | D | 167 | D |
| 168 | D | 169 | D | 170 | D |
| 171 | B | 172 | D | 173 | D |
| 174 | B | 175 | D | 176 | D |
| 177 | D | 178 | D | 179 | B |
| 180 | B | 181 | B | 182 | D |
| 183 | B | 184 | B | 185 | A |
| 186 | B | 187 | A | 188 | A |
| 189 | A | 190 | B | 191 | A |
| 192 | A | 193 | A | 194 | A |
| 194A | A | 194B | D | 195 | A |
| 196 | A | 197 | A | 198 | A |
| 199 | A | 200 | B | 201 | A |
| 201A | D | 202 | A | 203 | A |
| 204 | A | 205 | A | 206 | B |
| 207 | A | 208 | A | 209 | A |
| 210 | A | 211 | A | 212 | A |
| 213 | A | 214 | B | 215 | A |
| 216 | A | 217 | B | 218 | A |
| 219 | A | 220 | A | 221 | B |
| 222 | A | 223 | A | 224 | A |
| 225 | B | 226 | A | 226A | D |
| 226B | A | 227 | A | 228 | B |
| 229 | B | 230 | B | 231 | A |
| 232 | B | 233 | B | 234 | A |
| 234A | D | 235 | A | 236 | B |
| 237 | A | 237A | C | 238 | A |
| 239A | A | 239B | D | 240 | A |
| 241 | A | 242 | A | 243 | A |
| 244 | A | 245 | B | 246 | A |
| 247 | A | 248 | B | 249 | A |
| 250 | A | 251 | A | 252 | A |
| 253 | A | 254 | A | 255 | A |
| 256 | A | 257 | A | 258 | A |
| 259 | A | 260 | B | 261 | A |
| 262 | B | 263 | A | 264 | A |
| 265 | A | 266 | A | 267 | A |
| 268 | B | 269 | A | 270 | A |
| 270A | B | 271 | A | 272 | A |
| 273 | A | 274 | A | 275 | A |
| 276 | A | 277 | A | 278 | A |
| 279 | A | 280 | A | 281 | A |
| 282 | A | 283 | A | 284 | A |
| 285 | A | 286 | A | 287 | A |
| 288 | A | 289 | A | 290 | A |
| 291 | A | 292 | A | 293 | B |
| 294 | A | 295 | A | 296 | A |
| 297 | A | 298 | A | 299 | A |
| 300 | A | 301 | A | 302 | A |
| 302A | D | 302B | D | 302C | B |
| 303 | B | 304A | A | 304 | A |
| 304B | C | 305 | A | 306 | A |
| 307 | A | 308 | B | 309 | A |
| 310 | A | 311 | A | 312 | A |
| 312A | A | 312B | D | 313 | A |
| 314 | A | 315 | A | 316 | A |
| 317 | A | 318 | A | 319 | A |
| 320 | A | 320A | D | 321 | A |
| 322 | A | 323 | A | 323A | D |
| 324 | A | 325 | A | 326 | A |
| 327 | A | 328 | A | 329 | A |
| 330 | A | 331 | A | 332 | A |
| 333 | A | 334 | A | 335 | A |
| 336 | A | 337 | A | 338 | A |
| 339 | A | 340 | A | 341 | A |
| 341A | A | 341B | D | 342 | A |
| 343 | A | 344 | A | 345 | A |
| 346 | A | 347 | A | 347A | D |
| 348 | A | 349 | A | 350 | B |
| 351 | A | 352 | A | 353 | A |
| 354 | A | 355 | A | 355A | A |
| 355B | D | 356rac | A | 356 | A |
| 356A | D | 357 | A | 358 | D |
| 359 | A | 360 | A | 361 | A |
| 362 | A | 363 | D | 364 | A |
| 365 | A | 366 | A | 367 | A |
| 368 | D | 369 | A | 370 | A |
| 371 | A | 372 | A | 373 | A |
| 374 | A | 375 | A | 376 | A |
| 377 | A | 378 | D | 379 | A |
| 380 | A | 381 | D | 382 | A |
| 383 | D | 384 | B | 385 | D |
| 386 | A | 387 | A | 388 | D |
| 389 | A | 390 | D | 391 | A |
| 1A | D | 2A | D | 3A | D |
| 4A | D | 5A | A | 6A | D |
| 7A | D | 8A | D | 9A | D |
| 10A | C | 11A | B | 12A | C |
| 13A | D | 14A | D | 15A | D |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula 1: 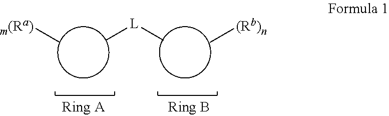
a tautomer thereof, a solvate or stereoisomer of the compound or the tautomer, or a pharmaceutically acceptable salt of the foregoing, wherein:
Ring A is phenyl, a 5- to 10-membered heteroaryl ring, a 5 to 10-membered carbocyclic ring or a 5 to 10-membered heterocyclic ring;
Ring B is phenyl, a 9- to 11-membered aryl ring, a 9- to 11-membered heteroaryl ring, or a 9- to 11-membered heterocyclic ring;
R^{a} is selected from ═O, C₃-C₆ cycloalkyl, —O(C₁-C₄ alkyl), COOH, CN, CONH₂, —C(═O)NH(C₁-C₄ alkyl), —C(═O)N(C₁-C₄ alkyl)₂, —C(═O)O(C₁-C₄ alkyl), halogen, and C₁-C₄ alkyl optionally substituted with 1 to 3 groups selected from halogen, —OH, cyclopropyl, oxetanyl, and azetidinyl;
R^{b} is selected from ═O, OH, halogen, CN, —C(═O)NH₂, —C(═O)NH(C₁-C₄ alkyl), —C(═O)O(C₁-C₄ alkyl), optionally substituted C₃-C₆ cycloalkyl, optionally substituted C₃-C₆ heterocyclyl, optionally substituted C₅-C₆ heteroaryl, —O(C₁-C₄ alkyl), —C(═O)(C₁-C₄ alkyl), —C(═O)(optionally substituted C₃-C₆ cycloalkyl), —C(═O)(optionally substituted 5- to 6-membered heterocyclyl), —C(═O)(optionally substituted 5- to 6-membered heteroaryl), —C(═O)(CH₂)pC(═O)NH₂, —NHC(═O)(C₁-C₄ alkyl), —C(═O)N(C₁-C₄ alkyl)₂, —C(═O)NH(CH₂)pOH, —S(═O)₂NH₂, 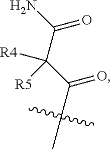
C₁-C₄ alkyl optionally substituted with 1 to 3 groups selected from halogen, NH₂, OH, OCH₃, —C(═O)NH(C₁-C₄ alkyl), —C(═O)O(C₁-C₄ alkyl), —C(═O)N(C₁-C₄ alkyl)₂, —COOH, —C(═O)NH₂, CN, C₃-C₅ cycloalkyl, phenyl, —C(═O)(C₁-C₄ alkyl), —C(═O)(C₃-C₆ cycloalkyl), —C(═O)(5- to 6-membered heterocyclyl), —C(═O)(optionally substituted 5- to 6-membered heteroaryl), C(═O)NH(CH₂)pOH, and 5- to 6-membered heteroaryl,
5- to 6-membered heteroaryl optionally substituted with 1 to 2 groups selected from C₁-C₃ alkyl, ═O, —NH₂, —CN, —CONH₂, halogen, and
4- to 6-membered heterocyclyl optionally substituted with 1 to 2 groups selected from ═O, —OH, —NH₂, —CN, —CONH₂, halogen, and C₁-C₃ alkyl, or two R^{b} attached to the same position on Ring B join to form a C₃-C₆ cycloalkyl, wherein R₄ and R₅ are each independently selected from H and C₁ to C₃ alkyl or R₄ and R₅ join to form a 3- to 5-membered cycloalkyl;
L is selected from 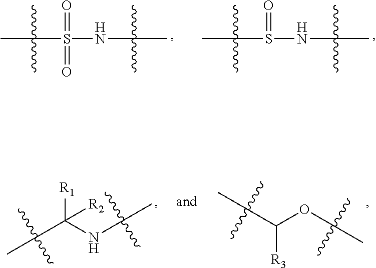
wherein
R₁ is H, D, or C₁-C₆ alkyl, and R₂ is selected from CN, C₁-C₆ alkenyl, C₃-C₆ cycloalkyl, C₃-C₆ heterocyclyl, 5- or 6-membered heteroaryl,
phenyl optionally substituted with 1 to 3 groups selected from C₁-C₄ alkyl and halogen, and
C₁-C₆ alkyl optionally substituted with 1 to 3 groups selected from halogen, OH, CN, —SO₂CH₃, —NHSO₂CH₃, —CONH₂, OCH₃, and phenyl, or
R₁ and R₂ join to form a 3- to 6-membered carbocyclyl or 3- to 6-membered heterocyclyl;
R₃ is C₁-C₄ alkyl optionally substituted with 1 to 3 groups selected from halogen, O(C₁-C₃ alkyl), OH, and NH₂;
m is an integer selected from 0, 1, 2, 3, and 4;
n is an integer selected from 0, 1, 2, 3, and 4, and
p is an integer selected from 1, 2, 3, and 4.
provided that the compound is not any of Compound 1A to Compound 121A.

2. The compound of claim 1, wherein the compound has the following structural Formula 14a: 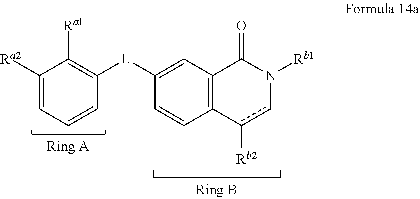
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H, CH₂CN, and C₁ to C₂ alkyl, R^{b2} is selected from H, halogen, CN, OCH₃, C₁ to C₃ alkyl, and C₃ to C₅ cycloalkyl, and L is selected from

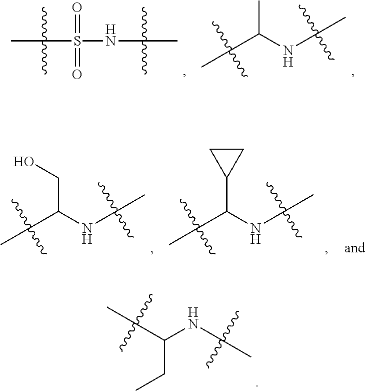

3. The compound of claim 1, wherein the compound has the following structural Formula 14b: 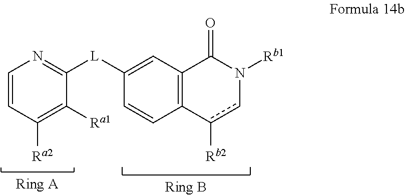
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H and C₁ to C₂ alkyl, R^{b2} is selected from H, halogen, CN, OCH₃, C₁ to C₃ alkyl, and C₃ to C₅ cycloalkyl, and L is selected from

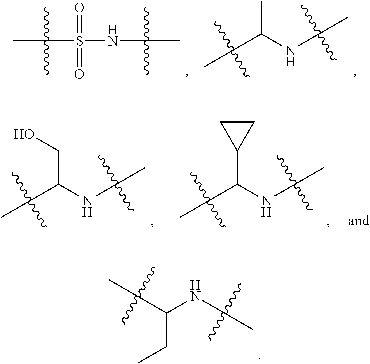

4. The compound of claim 1, wherein the compound has the following structural Formula 14c: 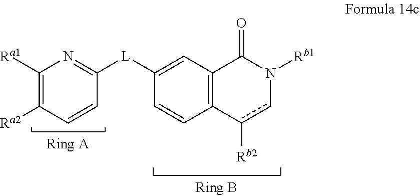
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H and C₁ to C₂ alkyl optionally substituted with C₃-C₄ cycloalkyl, R^{b2} is selected from H, halogen, and C₁ to C₃ alkyl, and L is selected from

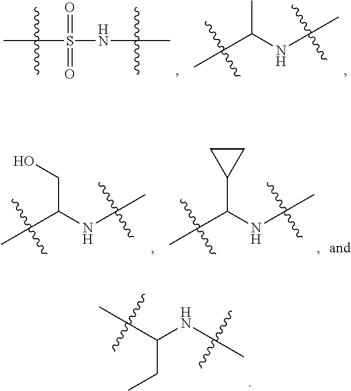

5. The compound of claim 1, wherein the compound has the following structural Formula 14d: 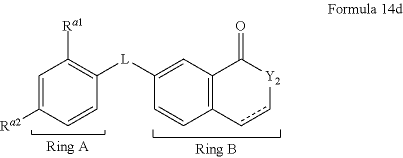
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

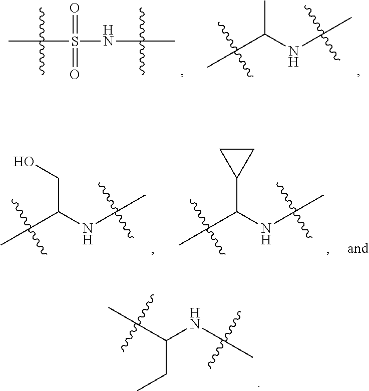

6. The compound of claim 1, wherein the compound has the following structural Formula 14e: 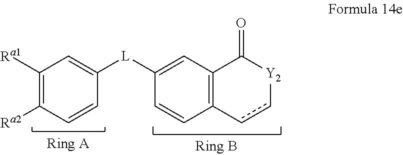
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

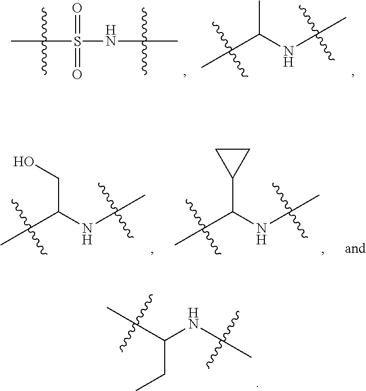

7. The compound of claim 1, wherein the compound has the following structural Formula 14f: 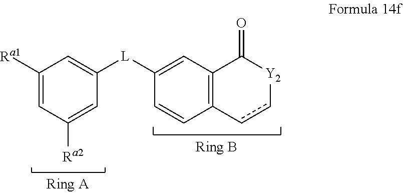
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

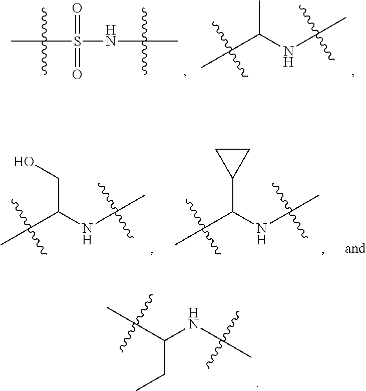

8. The compound of claim 1, wherein the compound has the following structural Formula 14g: 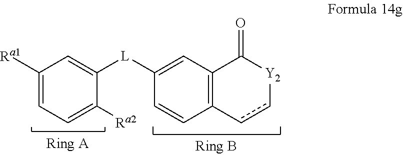
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, Y₂ is selected from N and O, and L is selected from

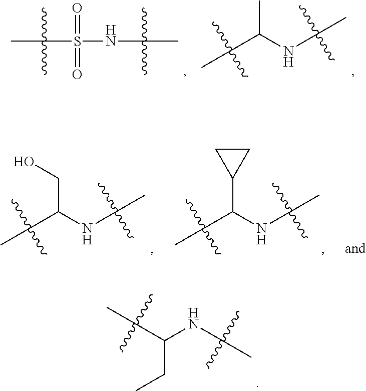

9. The compound of claim 1, wherein the compound has the following structural Formula 14h-1 or 14h-2: 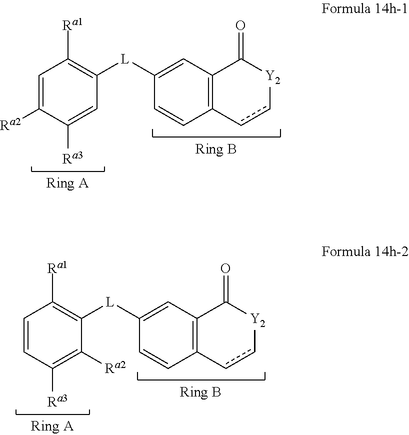
a tautomer thereof, a solvate or stereoisomer of the compound or the tautomer, or a pharmaceutically acceptable salt of the foregoing, wherein R^{a1}, R^{a2}, and R^{a3} are independently selected from F, Cl, Br, methyl, and OCH₃, Y₂ is selected from N and O, and L is selected from

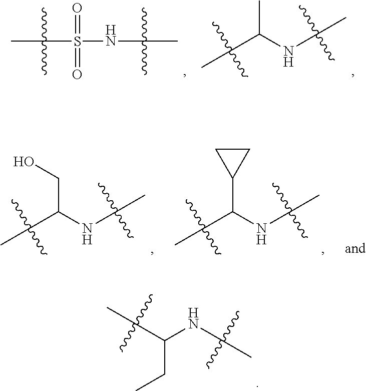

10. The compound of claim 1, wherein the compound has the following structural Formula 14i: 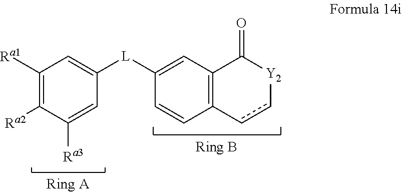
a tautomer thereof, a solvate or stereoisomer of the compound or the tautomer, or a pharmaceutically acceptable salt of the foregoing, wherein R^{a1}, R^{a2} and R^{a3} are independently selected from F, Cl, Br, methyl, and OCH₃, Y₂ is selected from N and O, and L is selected from

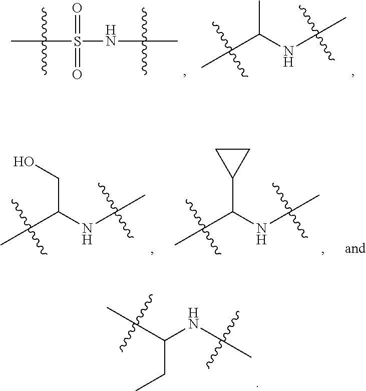

11. The compound of claim 1, wherein the compound has the following structural Formula 14j: 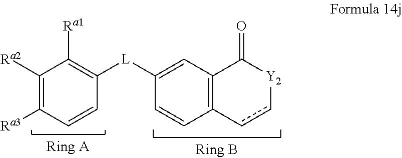
a tautomer thereof, a solvate or stereoisomer of the compound or the tautomer, or a pharmaceutically acceptable salt of the foregoing, wherein R^{a1}, R^{a2} and R^{a3} are independently selected from F, Cl, Br, methyl, and OCH₃, Y₂ is selected from N and O, and L is selected from

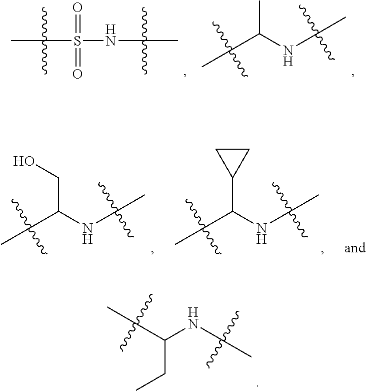

12. The compound of claim 1, wherein the compound has the following structural Formula 15a: 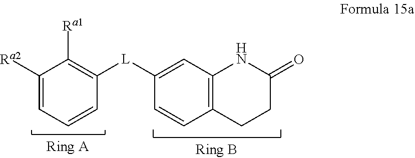
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, and L is selected from

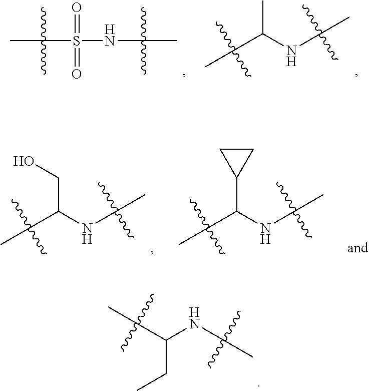

13. The compound of claim 1, wherein the compound has the following structural Formula 16-1 or 16-2: 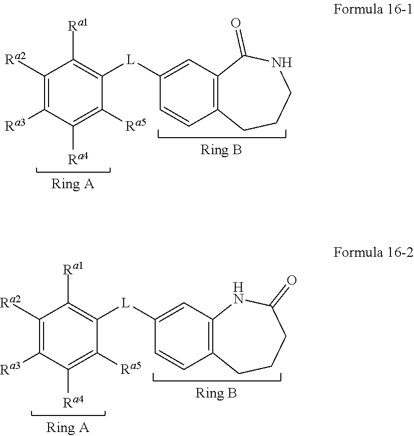
a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H.

14. The compound of claim 1, wherein the compound has the following structural Formula 16-1a or 16-2a: 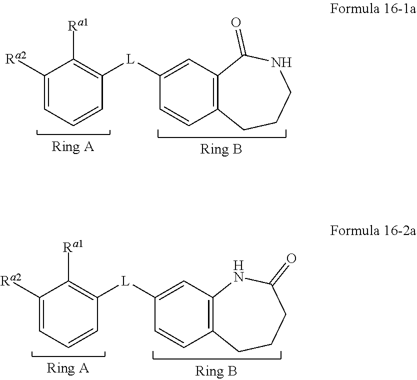
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, and L is selected from

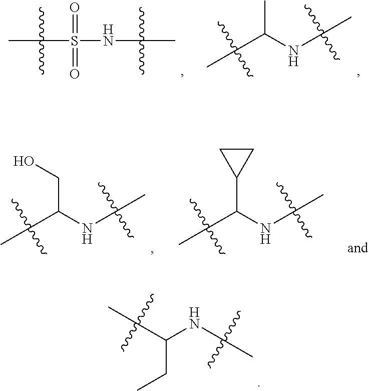

15. The compound of claim 1, wherein the compound has the following structural Formula 17: 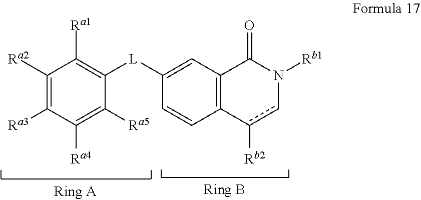
a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, R^{b1} is selected from H and C₁ to C₂ alkyl, and R^{b2} is selected from H and C₁ to C₄ alkyl.

16. The compound of claim 1, wherein the compound has the following structural Formula 17a: 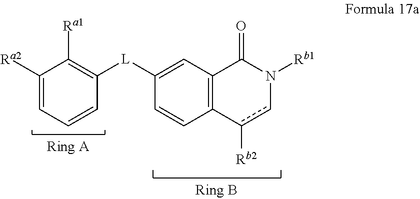
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H and C₁ to C₂ alkyl, R^{b2} is selected from H and C₁ to C₄ alkyl, and L is selected from

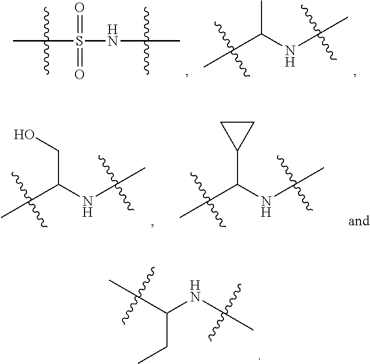

17. The compound of claim 1, wherein the compound has the following structural Formula 18: 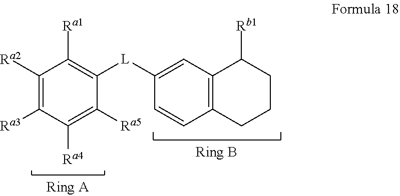
a tautomer thereof, a solvate or stereoisomer of the compound or the tautomer, or a pharmaceutically acceptable salt of the foregoing, wherein zero, one, two, or three of R^{a1}, R^{a2}, R^{a3}, R^{a4}, and R^{a5} are independently selected from F, Cl, Br, methyl, and OCH₃, and the rest of R^{a1}, R^{a2}, R^{a3}, R^{a4}, and R^{a5} are H, R^{b1} is selected from ═O, —NHCOCH₃, and OH.

18. The compound of claim 1, wherein the compound has the following structural Formula 18a: 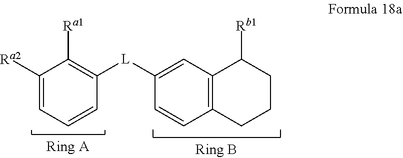
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from ═O, OH, and —NHCOCH₃, and L is selected from

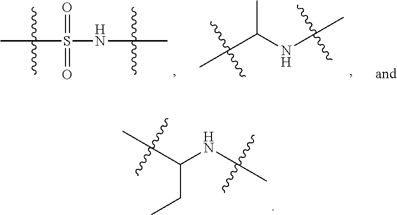

19. The compound of claim 1, wherein the compound has the following structural Formula 20: 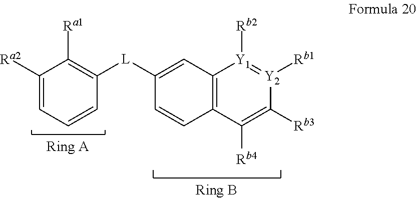
a tautomer thereof, a solvate or stereoisomer of the compound or the tautomer, or a pharmaceutically acceptable salt of the foregoing, wherein none or one of Y₁ and Y₂ is N and the other is C, R^{a1} and R^{a2} are independently selected from F, Cl, Br, methyl, and OCH₃, R^{b1} is selected from H and C₁ to C₂ alkyl provided that when Y₂ is N, R^{b1} is absent, R^{b2} is selected from H, CN, C₁ to C₂ alkyl, and —C(═O)NH₂, provided that when Y₁ is N, R^{b2} is absent, R^{b3} is selected from H and C₁ to C₂ alkyl, R^{b4} is selected from H and C₁ to C₂ alkyl, and L is selected from

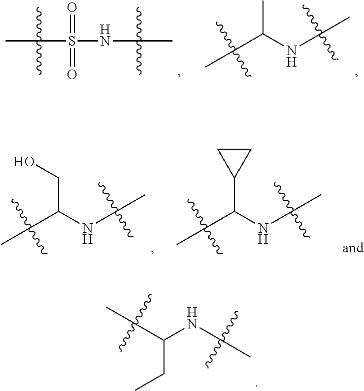

20. The compound of claim 1, wherein the compound has the following structural Formula 21a: 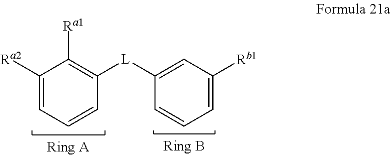
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from 5- to 6-membered heteroaryl and 5- to 6-membered heterocyclyl optionally substituted with 1 to 2 groups selected from ═O and C₁-C₃ alkyl, and L is selected from

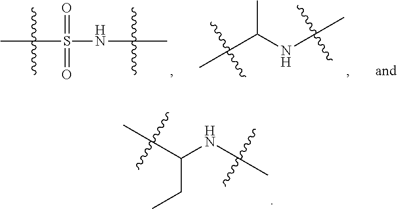

21. The compound of claim 1, wherein the compound has the following structural Formula 21b: 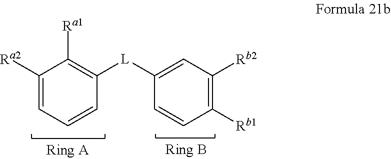
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from —C(═O)NH(C₁-C₄ alkyl), OCH₃, and —C(═O)O(C₁-C₄ alkyl), R^{b2} is selected from H and —C(═O)NH₂, and L is selected from

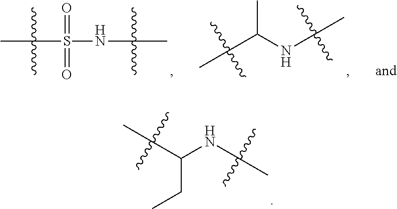

22. The compound of claim 1, wherein the compound has the following structural Formula 22a: 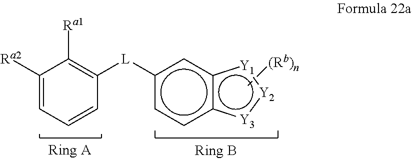
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, zero, one, or two of Y₁, Y₂, and Y₃ are N and the rest of Y₁, Y₂, and Y₃ are C, R^{b} is selected from C₁ to C₂ alkyl, C₃ to C₅ cycloalkyl, CN, CONH₂, and halogen, n is 0, 1, or 2, and L is selected from

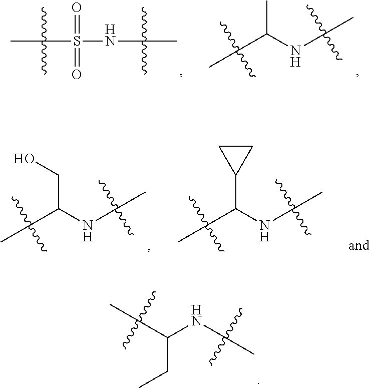

23. The compound of claim 1, wherein the compound has the following structural Formula 23a: 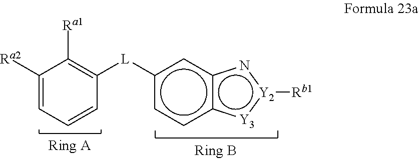
a tautomer thereof, a solvate or stereoisomer of the compound or the tautomer, or a pharmaceutically acceptable salt of the foregoing, wherein R^{a1} and R^{a2} are independently selected from F, Cl, Br, methyl, and OCH₃, R^{b1} is selected from H, C₁ to C₃ alkyl, halogen, C₃-C₅ cycloalkyl, CN, and CONH₂ provided that when Y₂ is S or O, R^{b1} is absent, one of Y₂ and Y₃ is S or O, and the other is C, and L is selected from

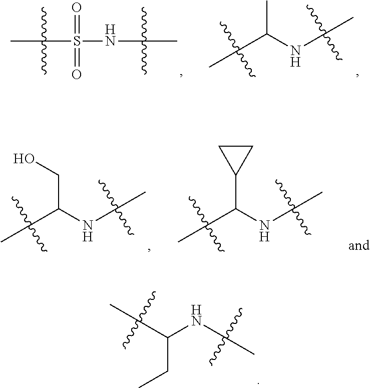

24. The compound of claim 1, wherein the compound has the following structural Formula 24: 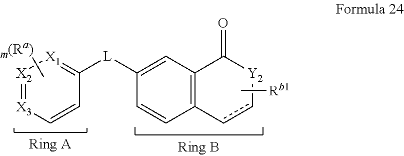
a tautomer thereof, a solvate or stereoisomer of the compound or the tautomer, or a pharmaceutically acceptable salt of the foregoing, wherein one of X₁, X₂, and X₃ is N and the other two of them are C, Y₂ is selected from C, N, and O, and R^{b1} is selected from C₁ to C alkyl, C₃ to C₅ cycloalkyl, O(C₁-C₃ alkyl), and halogen, and wherein L is

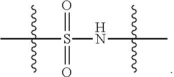 [SMILES: *NS(*)(=O)=O]

25. The compound of claim 1, wherein the compound has the following structural Formula 28-1 or 28-2: 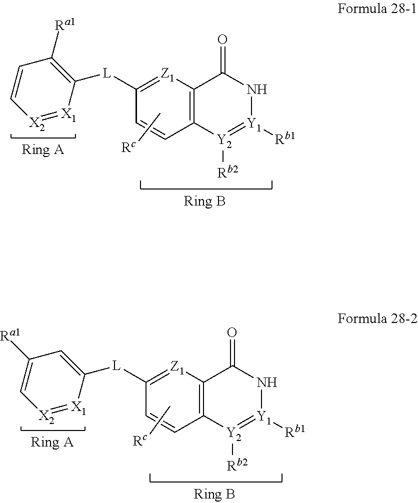
a tautomer thereof, a solvate or stereoisomer of the compound or the tautomer, or a pharmaceutically acceptable salt of the foregoing, wherein X₁, X₂, Y₁, Y₂, and Z₁ are independently selected from C and N, R^{a1} is selected from F, Cl, Br, methyl, CF₃, CF₂H, CN, CONH₂, CH₂NH₂, ethyl, and cyclopropyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{c} is H, halogen, CN, or methyl.

26. The compound of claim 1, wherein the compound has the following structural Formula 29-1 or 29-2: 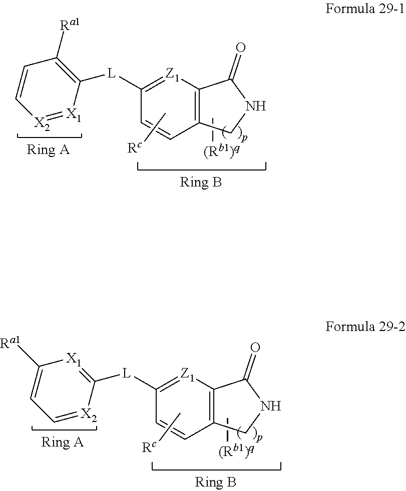
a tautomer thereof, a solvate or stereoisomer of the compound or the tautomer, or a pharmaceutically acceptable salt of the foregoing, wherein X₁, X₂, and Z₁ are independently selected from C and N, R^{a1} is selected from F, Cl, Br, methyl, CF₃, CF₂H, CN, CONH₂, CH₂NH₂, ethyl, and cyclopropyl, R^{b1} is selected from F, Cl, Br, methyl, CN, CF₃, CF₂H, ethyl, CH₂OH, cyclopropyl, and OCH₃, wherein R^{b1} is absent or C₁ to C₄ alkyl when connected to N in Ring B, R^{c} is H, halogen, CN, or methyl, p is 1, 2, or 3, and q is 0, 1, or 2.

27. The compound of claim 1, wherein the compound has the following structural Formula 30-1, 30-2, 30-3, 30-4, or 30-5: 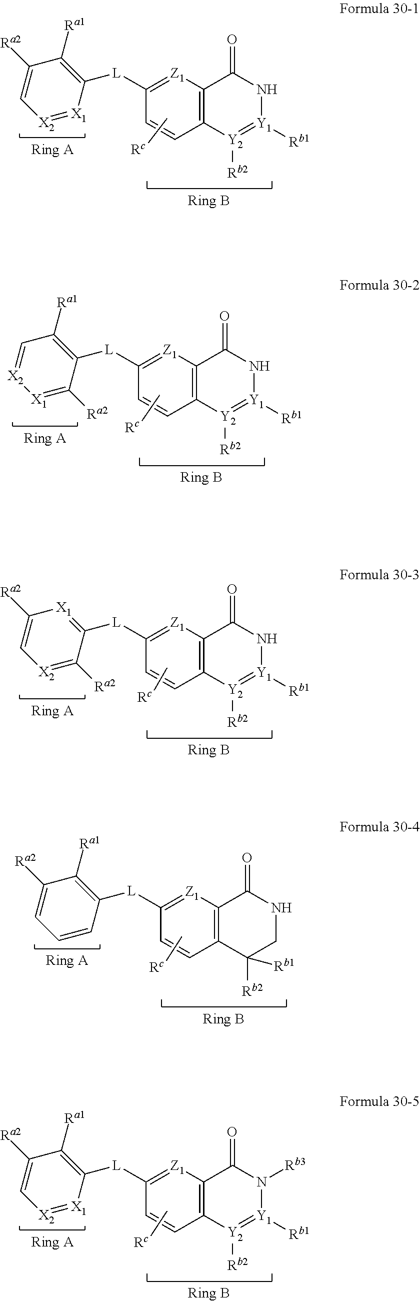
a tautomer thereof, a solvate or stereoisomer of the compound or the tautomer, or a pharmaceutically acceptable salt of the foregoing, wherein X₁, X₂, Y₁, Y₂, and Z₁ are independently selected from C and N, R^{a1} and R^{a2} are each independently selected from F, Cl, Br, methyl, CF₃, CF₂H, ethyl, CN, CONH₂, CH₂NH₂, and cyclopropyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from C₁ to C₄ alkyl optionally substituted with —C(═O)NH₂, 3 to 5-membered cycloalkyl optionally substituted with OH, and 5-6 membered heteroaryl optionally substituted with 1-2 groups selected from F, Cl, Br, Me, CF₃, CF₂H, CN, CONH₂, and NH₂, and R^{c} is H, halogen, CN, or methyl.

28. The compound of claim 1, wherein the compound has the following structural Formula 31-1 or 31-2: 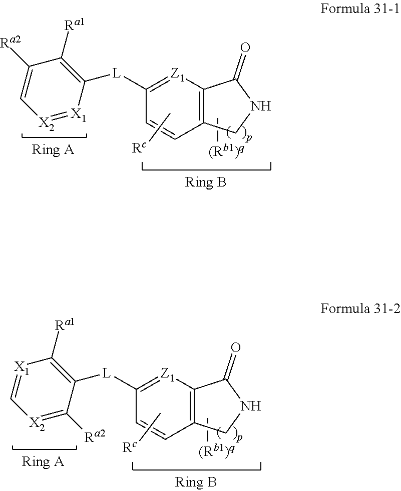
a tautomer thereof, a solvate or stereoisomer of the compound or the tautomer, or a pharmaceutically acceptable salt of the foregoing, wherein X₁, X₂, and Z₁ are independently selected from C and N, R^{a1} and R^{a2} are each independently selected from F, Cl, Br, methyl, CF₃, CF₂H, CN, CONH₂, CH₂NH₂, ethyl, and cyclopropyl, R^{b1} is selected from F, Cl, Br, methyl, CN, CF₃, CF₂H, ethyl, cyclopropyl, and OCH₃, wherein R^{b1} is absent or C₁ to C₄ alkyl when connected to N in Ring B, R^{c} is H, halogen, CN, or methyl, p is 1, 2, or 3, and q is 0, 1, or 2.

29. The compound of claim 1, wherein the compound has the following structural Formula 32-1, 32-2, 32-3, 32-4, or 32-5: 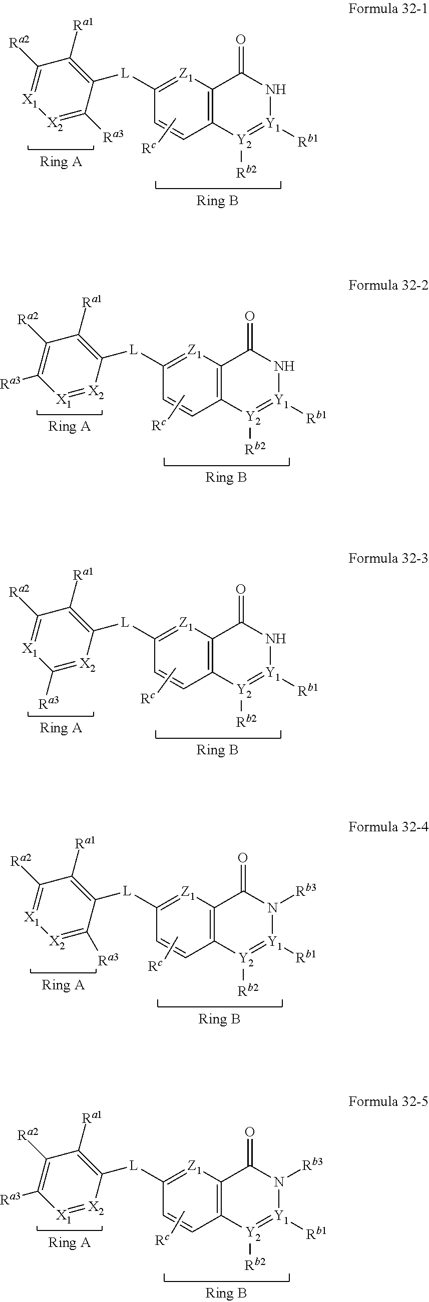
a tautomer thereof, a solvate or stereoisomer of the compound or the tautomer, or a pharmaceutically acceptable salt of the foregoing, wherein X₁, X₂, Y₁, Y₂, and Z₁ are independently selected from C and N, R^{a1}, R^{a2}, and R^{a3} are each independently selected from F, Cl, Br, methyl, CF₃, CF₂H, CN, CONH₂, CH₂NH₂, ethyl, cyclopropyl, and OCH₃, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from H, C₁ to C₄ alkyl optionally substituted with —C(═O)NH₂, 3 to 5-membered cycloalkyl optionally substituted with OH, and 5-6 membered heteroaryl optionally substituted with 1-2 groups selected from F, Cl, Br, Me, CF₃, CF₂H, CN, CONH₂, and NH₂, and R^{c} is H, halogen, CN, or methyl.

30. The compound of claim 1, wherein the compound has the following structural Formula 33-1, 33-2, or 33-3: 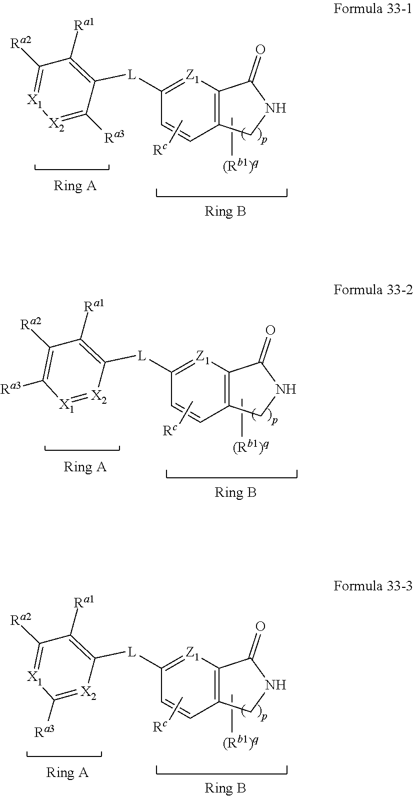
a tautomer thereof, a solvate or stereoisomer of the compound or the tautomer, or a pharmaceutically acceptable salt of the foregoing, wherein X₁, X₂, and Z₁ are independently selected from C and N, R^{a1}, R^{a2}, and R^{a3} are each independently selected from F, Cl, Br, methyl, CF₃, CF₂H, CN, CONH₂, CH₂NH₂, ethyl, cyclopropyl, and OCH₃, R^{b1} is selected from F, Cl, Br, methyl, CN, CF₃, CF₂H, ethyl, cyclopropyl, and OCH₃, wherein R^{b1} is absent or C₁ to C₄ alkyl when connected to N in Ring B, and R^{c} is H, halogen, CN, or methyl, p is 1, 2, or 3, and q is 0, 1, or 2.

31. The compound of claim 1, wherein the compound has the following structural Formula 34-1, 34-2, 34-3, 34-4, 34-5, or 34-6: 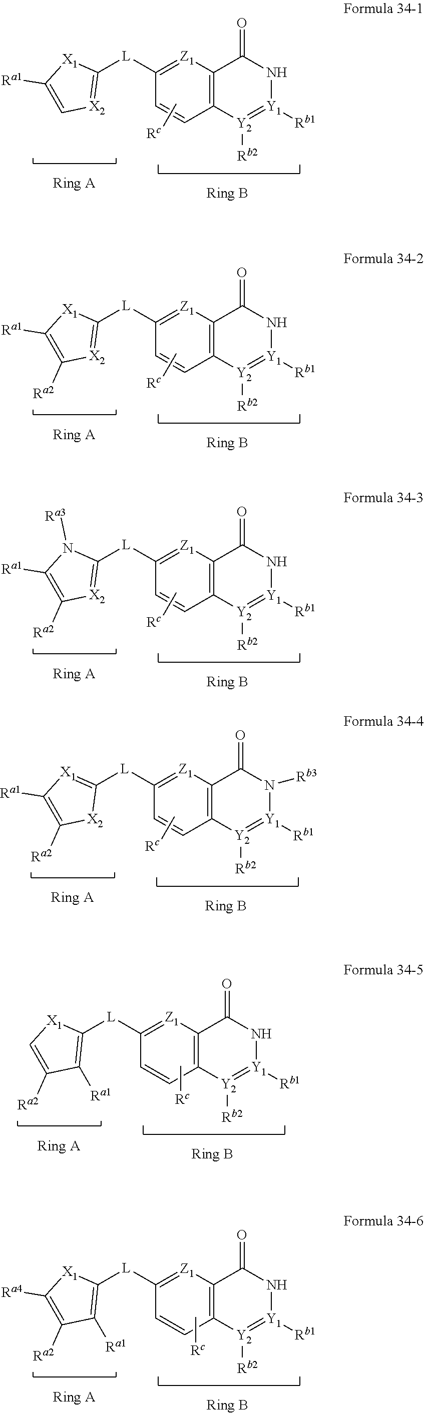
a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2}, and R^{a4} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from H, 5- to 6-membered heteroaryl optionally substituted with 1-2 groups selected from F, Cl, Br, Me, CF₃, CF₂H, CN, CONH₂, and NH₂, and C₁ to C₄ alkyl optionally substituted with —C(═O)NH₂, and R^{c} is H, halogen, CN, or methyl.

32. The compound of claim 1, wherein the compound has the following structural Formula 35-1, 35-2, 35-3, or 35-4: 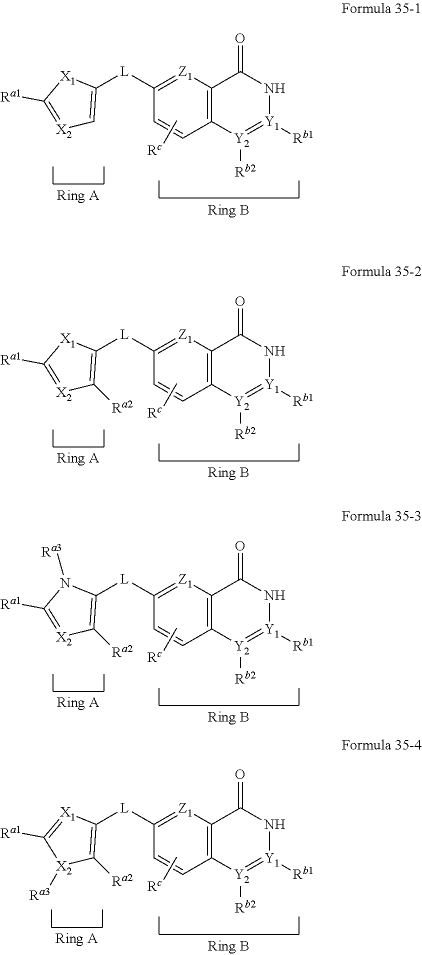
a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2} are each independently selected from F, Cl, Br, C₁ to C₄alkyl, CF₃, CF₂H, and OCH₃, R^{a3} is C₁ to C₄ alkyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{c} is H, halogen, CN, or methyl.

33. The compound of claim 1, wherein the compound has the following structural Formula 36-1, 36-2, or and 36-3: 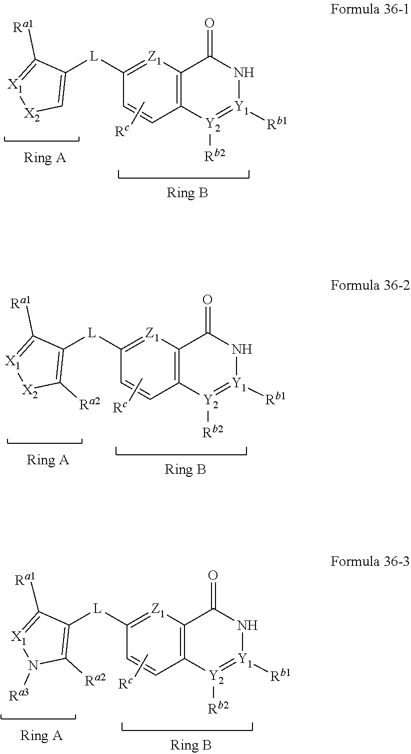
a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2} are each independently selected from F, Cl, Br, C₁ to C₄alkyl, CF₃, CF₂H, CN, CONH₂, CH₂NH₂, and OCH₃, R^{a3} is C₁ to C₄ alkyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{c} is H, halogen, CN, or methyl.

34. The compound of claim 1, wherein the compound has the following structural Formula 37-1, 37-2, 37-3, 37-4, 37-5, or 37-6: 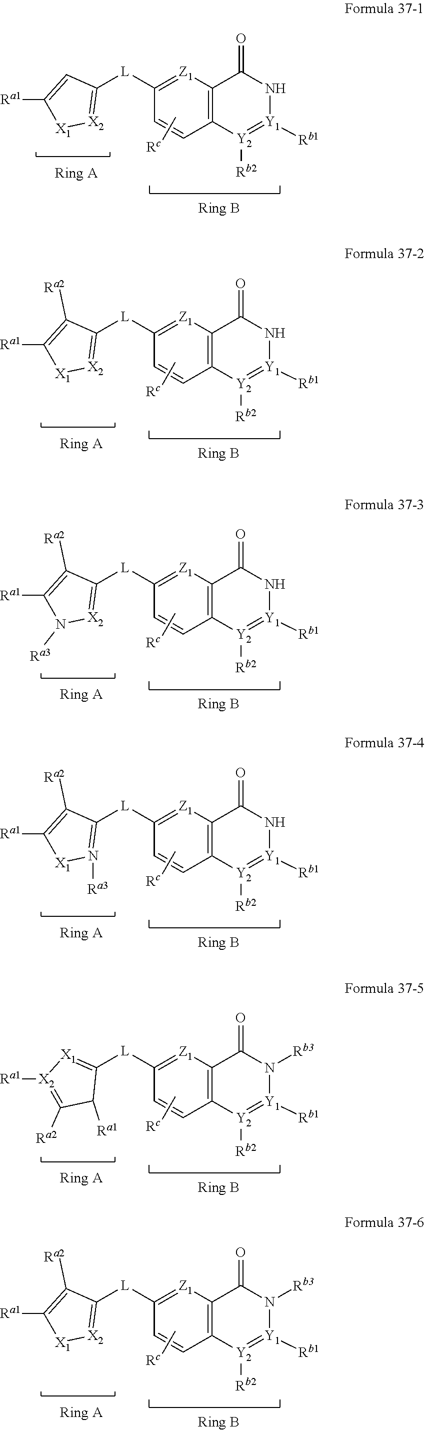
a tautomer thereof, a solvate or stereoisomer of the compound or the tautomer, or a pharmaceutically acceptable salt of the foregoing, wherein X₁ and X₂ are independently selected from N, O, and S, Y₁, Y₂, and Z₁ are independently selected from C and N, R^{a1} and R^{a2} are each independently selected from F, Cl, Br, C₁ to C₄ alkyl optionally substituted with 1-3 groups of halogen, C₃-C₄ cycloalkyl, CF₃, CF₂H, CN, CONH₂, CH₂NH₂, and OCH₃, R^{a3} is selected from C₃-C₄ cycloalkyl and C₁ to C₄ alkyl optionally substituted by 1 to 3 groups selected from halogen, cyclopropyl, and 4- to 5-membered heterocyclyl, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, R^{b3} is selected from 5- to 6-membered heteroaryl substituted with 1-2 groups selected from F, Cl, Br, Me, CF₃, CF₂H, CN, CONH₂, and NH₂, and C₁ to C₄ alkyl optionally substituted with OH or —C(═O)NH₂, and R^{c} is H, halogen, CN, or methyl.

35. The compound of claim 1, wherein the compound has the following structural Formula 38-1, 38-2, or 38-3: 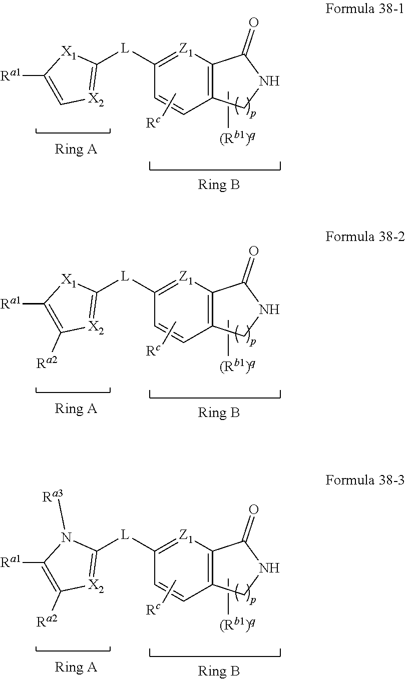
a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, and R^{c} is H, halogen, CN, or methyl, p is 1, 2, or 3, and q is 0, 1, or 2.

36. The compound of claim 1, wherein the compound has the following structural Formula 39-1, 39-2, 39-3, or 39-4: 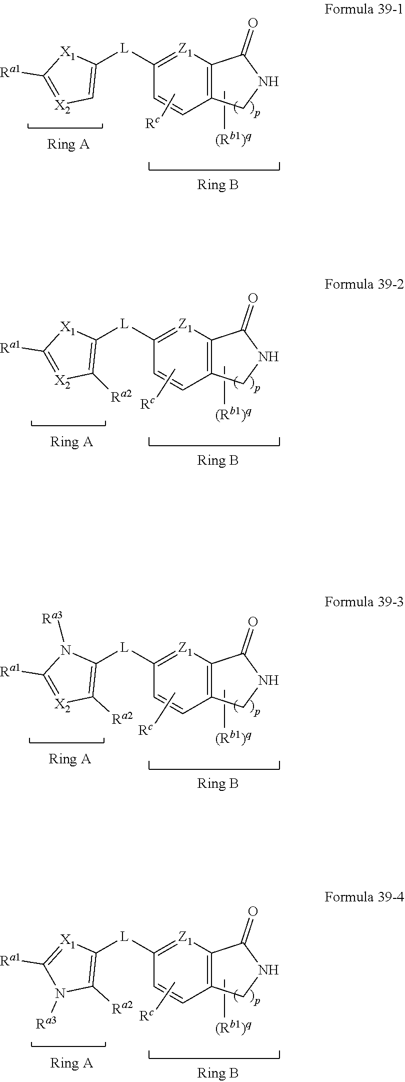
a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, R^{c} is H, halogen, CN, or methyl, p is 1, 2, or 3, and q is 0, 1, or 2.

37. The compound of claim 1, wherein the compound has the following structural Formula 40-1, 40-2, or 40-3: 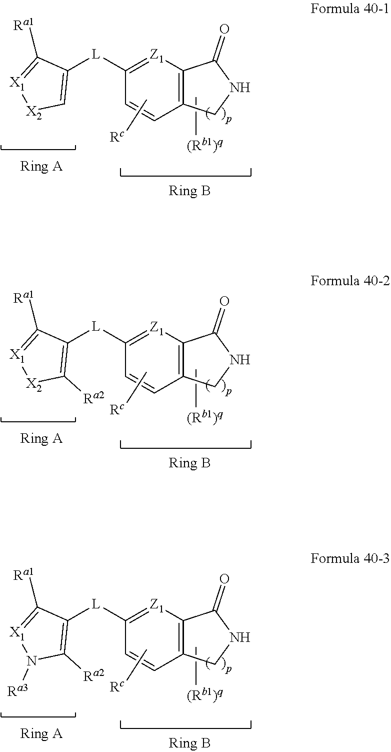
a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, R^{c} is H, halogen, CN or methyl, p is 1, 2, or 3, and q is 0, 1, or 2.

38. The compound of claim 1, wherein the compound has the following structural Formula 41-1, 41-2, 41-3, 41-4, or 41-5: 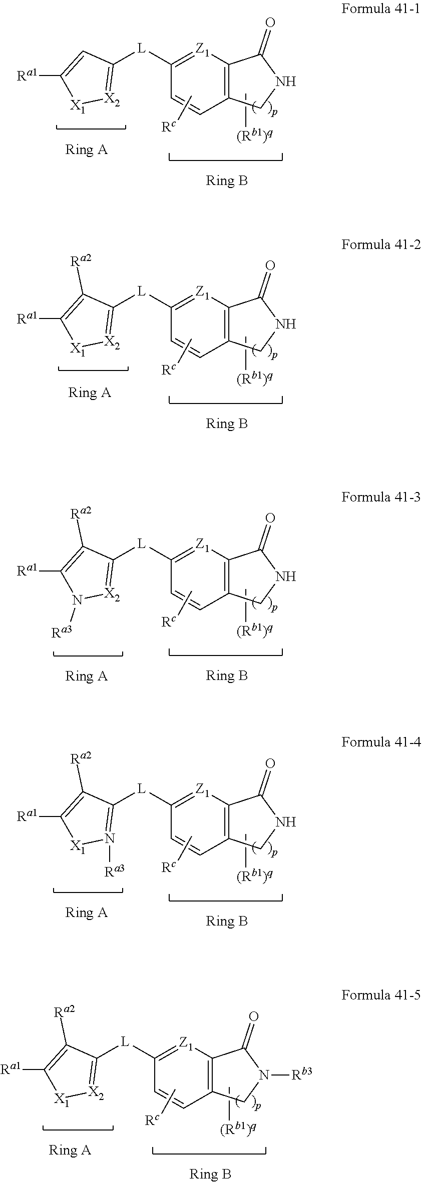
a tautomer thereof, a solvate or stereoisomer of the compound or the tautomer, or a pharmaceutically acceptable salt of the foregoing, wherein X₁ and X₂ are independently selected from N, O, and S, Z₁ is selected from C and N, R^{a1} and R^{a2} are each independently selected from F, Cl, Br, C₁ to C₄ alkyl, CF₃, CF₂H, CN, CONH₂, CH₂NH₂, and OCH₃, R^{a3} is C₁ to C₄ alkyl, R^{b1} is selected from F, Cl, Br, methyl, CN, CF₃, CF₂H, ethyl, cyclopropyl, and OCH₃, R^{b3} is selected from C₁ to C₄ alkyl optionally substituted with OH or —C(═O)NH₂, R^{c} is H, halogen, CN, or methyl, p is 1, 2, or 3, and q is 0, 1, or 2.

39. The compound of claim 1, wherein the compound has the following structural Formula 42-1, 42-2, or 42-3: 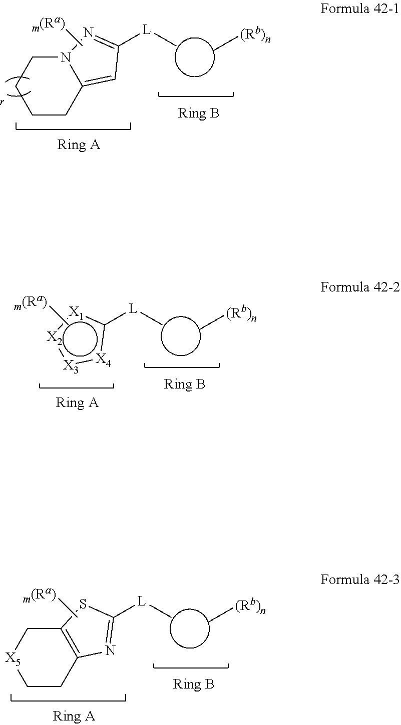
a tautomer thereof, a solvate or stereoisomer of the compound or the tautomer, or a pharmaceutically acceptable salt of the foregoing, wherein X₁, X₂, X₃, and X₄ are independently selected from C, N, and S, and wherein X₅ is selected from C and N, and r is an integer selected from 0, 1, and 2.

40. The compound of claim 1, wherein the compound has the following structural Formula 43-1, 43-2, 43-3, or 43-4: 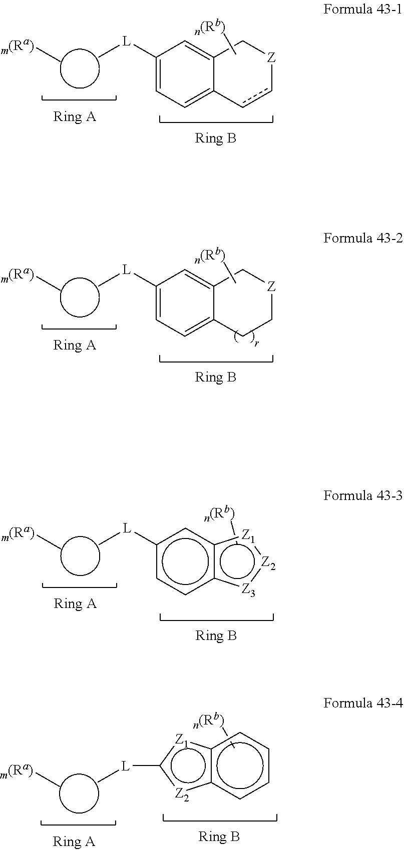
a tautomer thereof, a solvate or stereoisomer of the compound or the tautomer, or a pharmaceutically acceptable salt of the foregoing, wherein Z, Z₁, Z₂ and Z₃ are independently selected from C, S, and N, and r is an integer selected from 0, 1, and 2.

41. The compound of claim 1, wherein the compound has the following structural Formula 44-1, 44-2, 44-3, 44-4, 44-5, 44-6, 44-7, 44-8, 44-9, 44-10, 44-11, 44-12, 44-13, or 44-14: 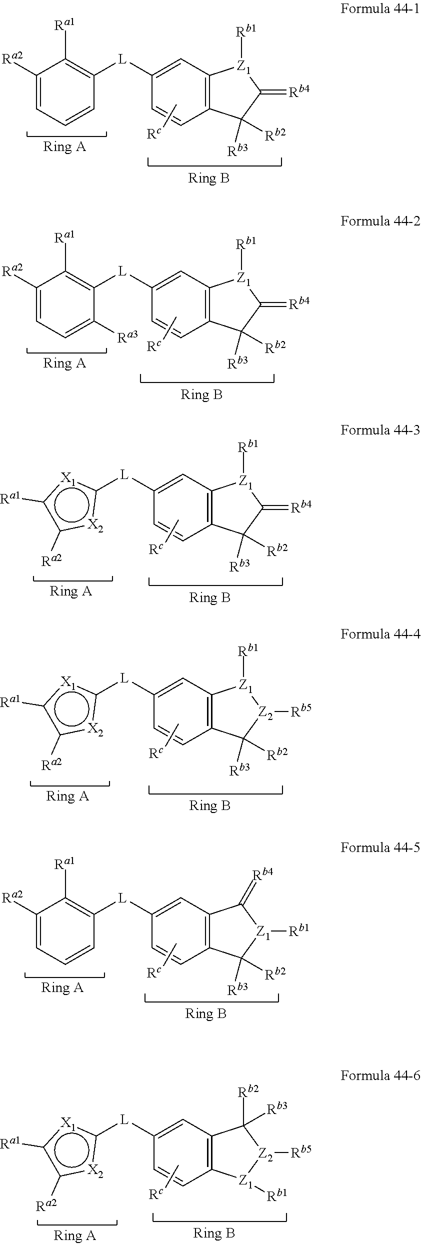 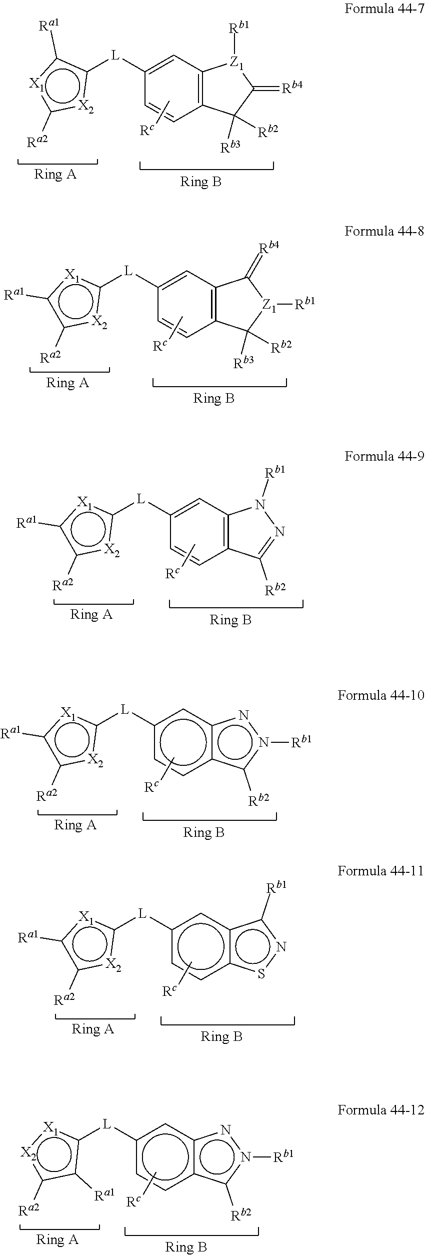 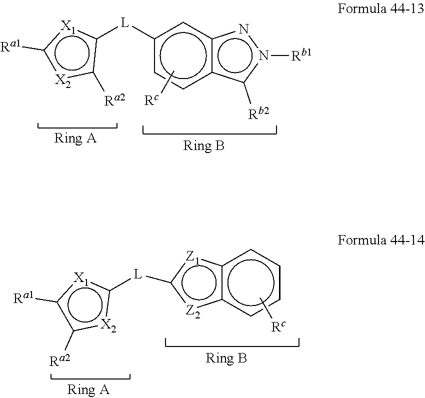
a tautomer thereof, a solvate or stereoisomer of the compound or the tautomer, or a pharmaceutically acceptable salt of the foregoing, wherein:
X₁ is selected from C, S, O and N, X₂ is selected from C, N, S, and O, Z₁ and Z₂ are independently selected from C, N, and S,
R^{a1}, R^{a2}, and R^{a3} are each independently selected from F, Cl, Br, C₁ to C₄ alkyl, CF₃, CF₂H, CN, CONH₂, CH₂NH₂, and OCH₃,
R^{b1} is selected from H, F, Cl, Br, methyl, CN, CF₃, CF₂H, ethyl, OCH₃, —C(═O)CH₂C(═O)NH₂, —C(═O)(C₁ to C₄ alkyl), —C(═O)(C₃ to C₅ cycloalkyl), —C(═O)(5- to 6-membered heteroaryl), —C(═O)(4- to 6-membered heterocyclyl optionally substituted with ═O or OH), —C(═O)OH, OH, 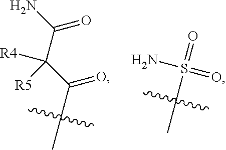
—C(═O)NH₂, 3- to 5-membered cycloalkyl optionally substituted by OH, and C₁ to C₄ alkyl optionally substituted by OH or —C(═O)NH₂,
R^{b2} and R^{b3} are independently selected from H, F, Cl, Br, methyl, CN, CF₃, CF₂H, ethyl, —C(═O)NH₂, and OCH₃, or R^{b2} and R^{b3} join to form a C₃ to C₅ cycloalkyl,
R^{b4} is ═O or absent,
R^{b5} is selected from H, F, Cl, Br, methyl, CN, CF₃, CF₂H, ethyl, OCH₃, —C(═O)CH₂C(═O)NH₂, —C(═O)(C₁ to C₄ alkyl), —C(═O)(C₃ to C₅ cycloalkyl), —C(═O)(5- to 6-membered heteroaryl), —C(═O)(5- to 6-membered heterocyclyl optionally substituted with ═O), —C(═O)OH, OH, C₁ to C₄ alkyl optionally substituted by OH or —C(═O)NH₂, and C₃ to C₅ cycloalkyl optionally substituted with OH, and
R^{c} is H, halogen, CN, or methyl, and
wherein R₄ and R₅ join to form a 3 to 4-membered cycloalkyl.

42. The compound of claim 1, wherein the compound has the following structural Formula 45-1 or 45-2: 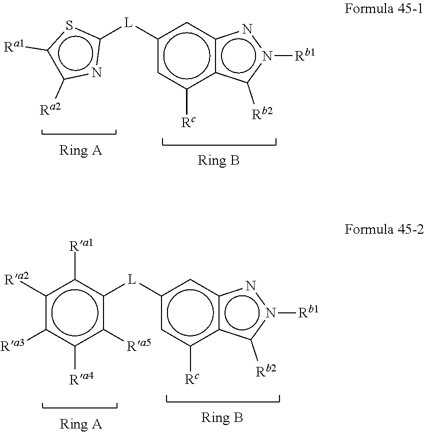
a tautomer thereof, a solvate or stereoisomer of the compound or the tautomer, or a pharmaceutically acceptable salt of the foregoing, wherein
R^{a1} and R^{a2} are each independently selected from halogen and C₁ to C₂ alkyl optionally substituted with 1 to 3 groups of halogen,
R′^{a1}, R′^{a2}, R′^{a3}, R′^{a4}, and R′^{a5} are each independently selected from H and halogen, wherein 2, 3, or 4 of R′^{a1}, R′^{a2}, R′^{a3}, R′^{a4}, and R′^{a5} are halogen and the rest is H,
R^{b1} is selected from H and C₁ to C₄ alkyl optionally substituted by —C(═O)NH₂,
R^{b2} is selected from H and C₁ to C₄ alkyl optionally substituted by 1-3 groups of halogen, and
R^{c} is selected from H, halogen, and C₁ to C₂alkyl.

43. The compound of claim 1, wherein the compound has the following structural Formula 46-1, 46-2, or 46-3: 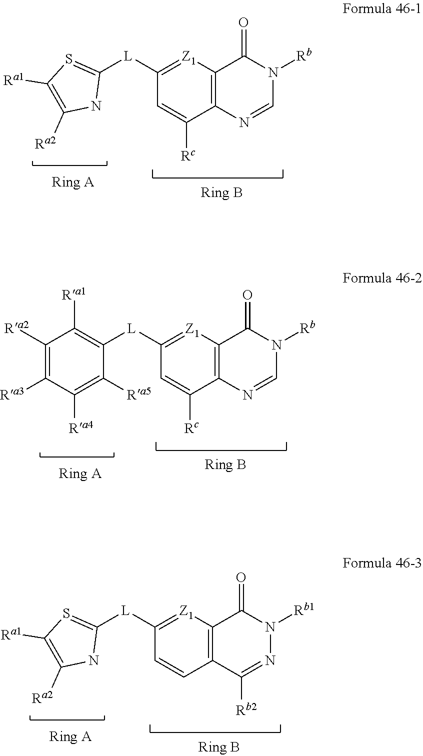
a tautomer thereof, a solvate or stereoisomer of the compound or the tautomer, or a pharmaceutically acceptable salt of the foregoing, wherein
Z₁ is selected from C and N,
R^{a1} and R² are each independently selected from halogen and C₁ to C₂ alkyl optionally substituted with 1 to 3 groups of halogen,
R′^{a1}, R′^{a2}, R′^{a3}, R′^{a4}, and R′^{a5} are each independently selected from H and halogen, wherein 2, 3, or 4 of R′^{a1}, R′^{a2}, R′^{a3}, R′^{a4}, and R′^{a5} are halogen and the rest is H,
R^{b} is selected from H and 5- to 6-membered heteroaryl containing 2 to 3 heteroatoms selected from N and S, wherein the 5- to 6-membered heteroaryl of R^{b} is optionally substituted by 1 to 3 groups selected from halogen and C₁ to C₄ alkyl,
R^{b1} is selected from H and C₁ to C₄ alkyl optionally substituted by —C(═O)NH₂,
R^{b2} is selected from H, halogen, —O(C₁ to C₄ alkyl), and C₁ to C₄ alkyl optionally substituted with 1-3 groups of halogen, and
R^{c} is selected from H, halogen, and C₁ to C₂alkyl.

44. The compound, tautomer, solvate, stereoisomer, or pharmaceutically acceptable salt of claim 1, wherein Ring A substituted with m groups of R^{a} is selected from: 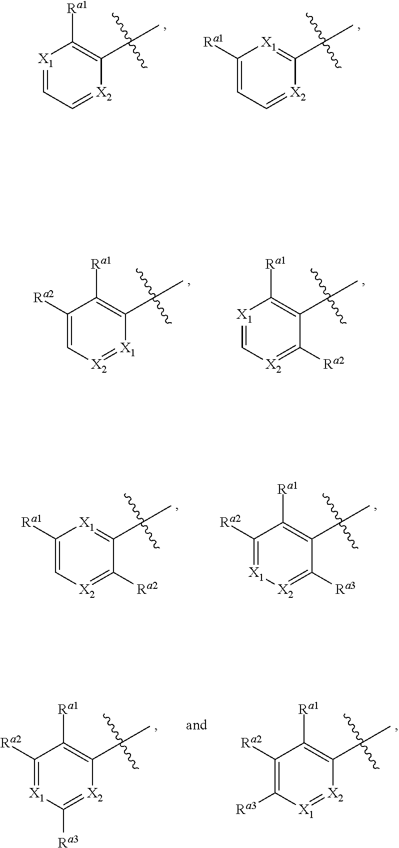
wherein R^{a1}, R^{a2}, and R^{a3} are independently selected from F, Cl, Br, methyl, CF₃, CF₂H, and cyclopropyl, and X₁ and X₂ are independently selected from C and N.

45. The compound, tautomer, solvate, stereoisomer, or pharmaceutically acceptable salt of claim 1, wherein Ring A substituted with m groups of R^{a} is selected from: 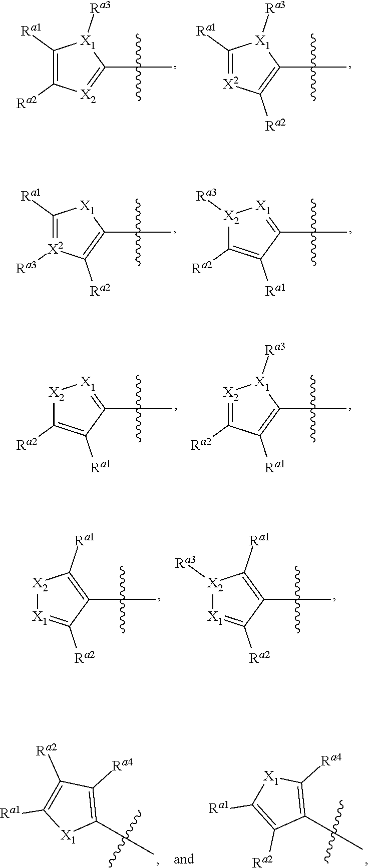
wherein R^{a1} and R^{a2} are independently selected from H, CN, F, Cl, Br, C₁ to C₄ alkyl, CF₃, CF₂H, and OCH₃, R^{a3} is H, or C₁ to C₄ alkyl, R^{a4} is selected from H, F, Cl, Br, and C₁ to C₄ alkyl, and X₁ and X₂ are independently selected from N, O, and S.

46. The compound, tautomer, solvate, stereoisomer, or pharmaceutically acceptable salt of claim 1, wherein Ring A is selected from: 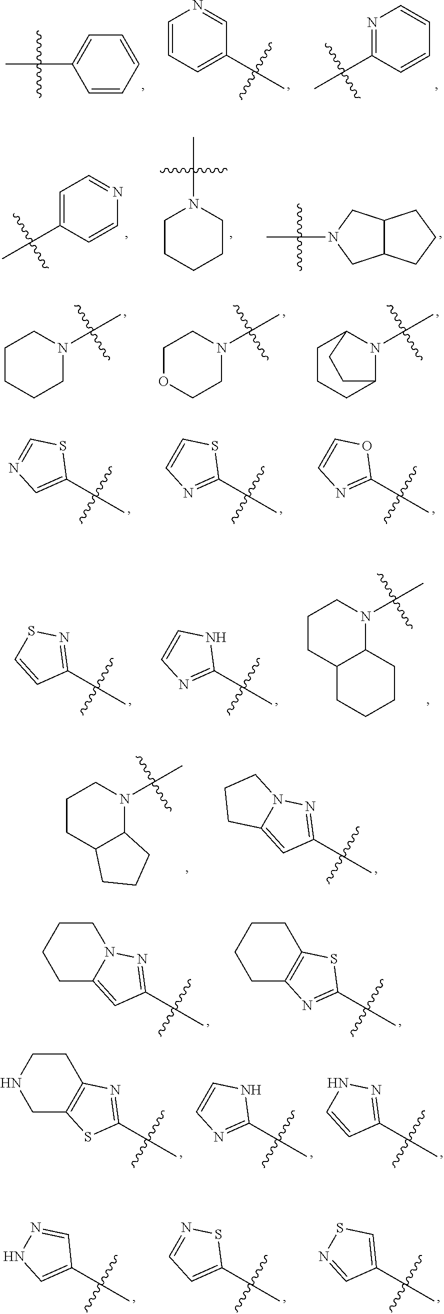 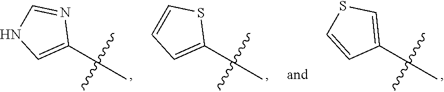 [SMILES: *c1c[nH]cn1 ; *c1cccs1 ; *c1ccsc1]
wherein Ring A is substituted with m groups of R^{a}.

47. The compound, tautomer, solvate, stereoisomer, or pharmaceutically acceptable salt of claim 1, wherein Ring A substituted with m groups of R^{a} is selected from:

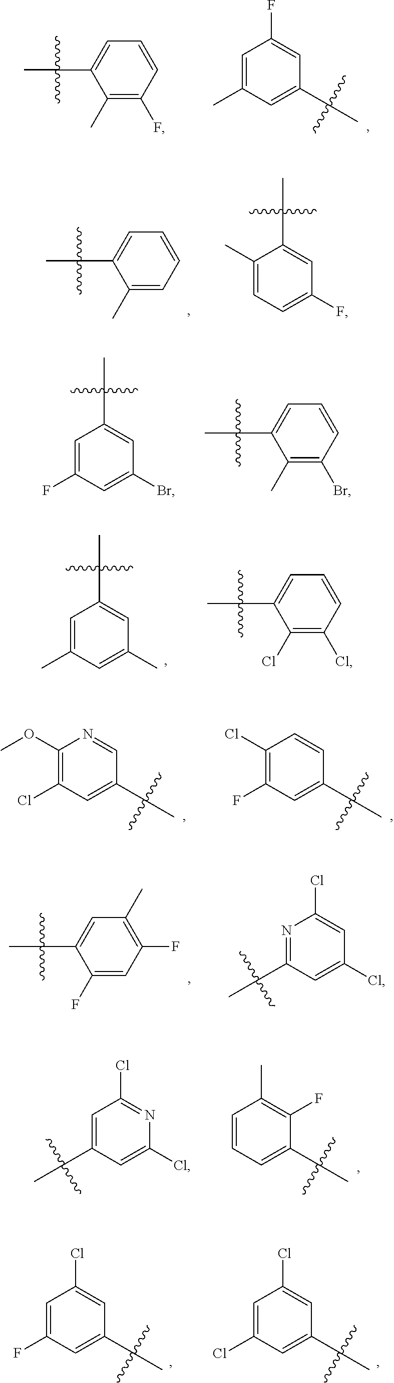

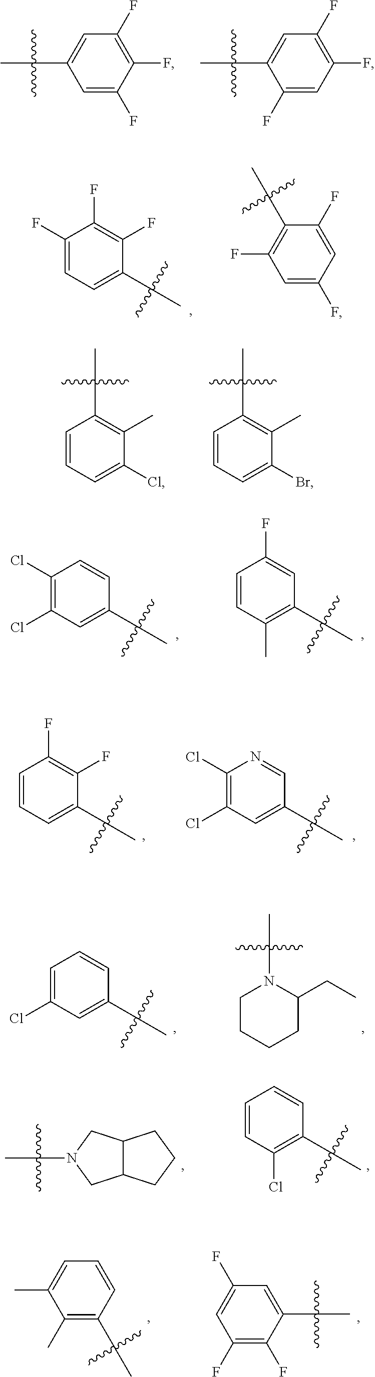

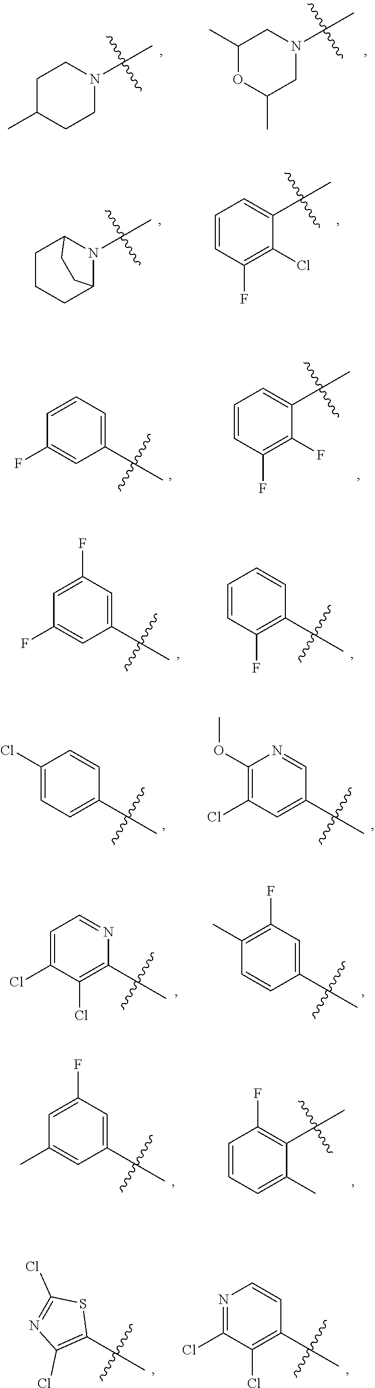

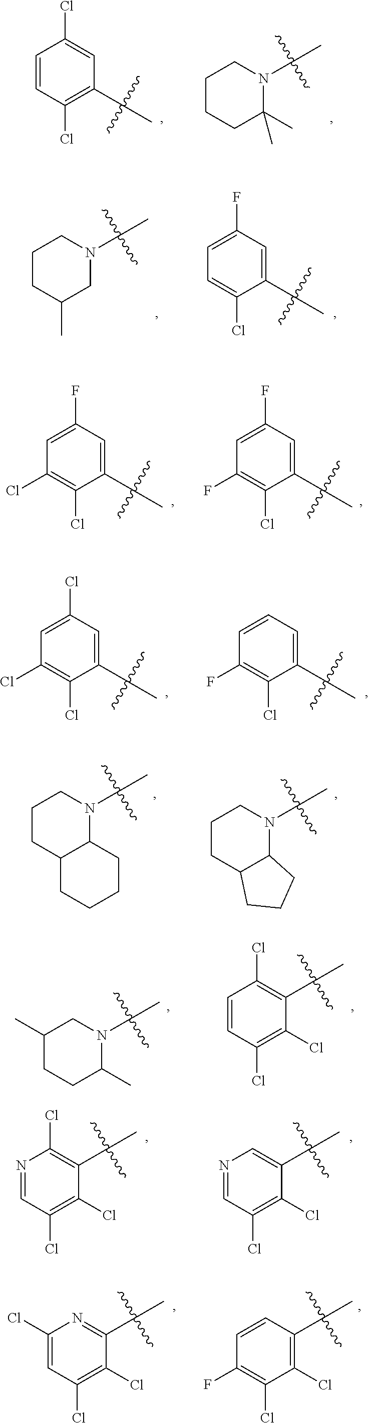

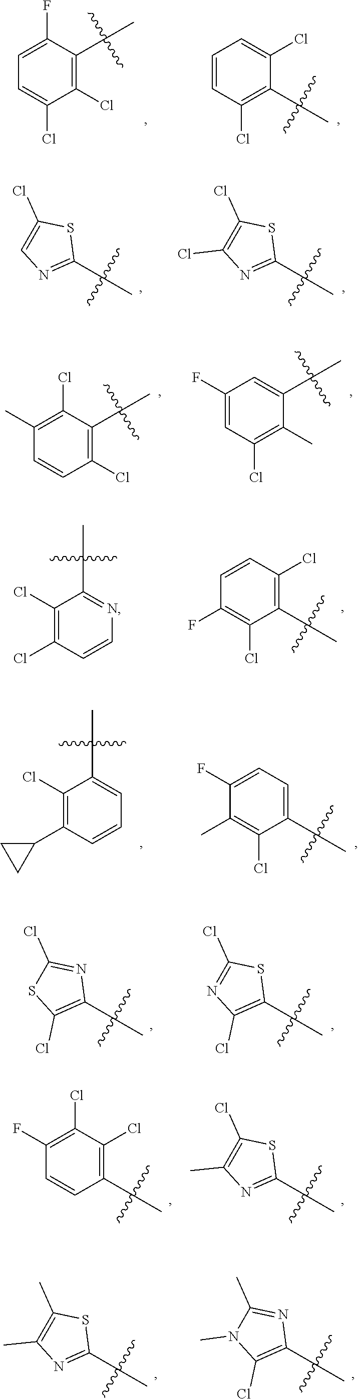

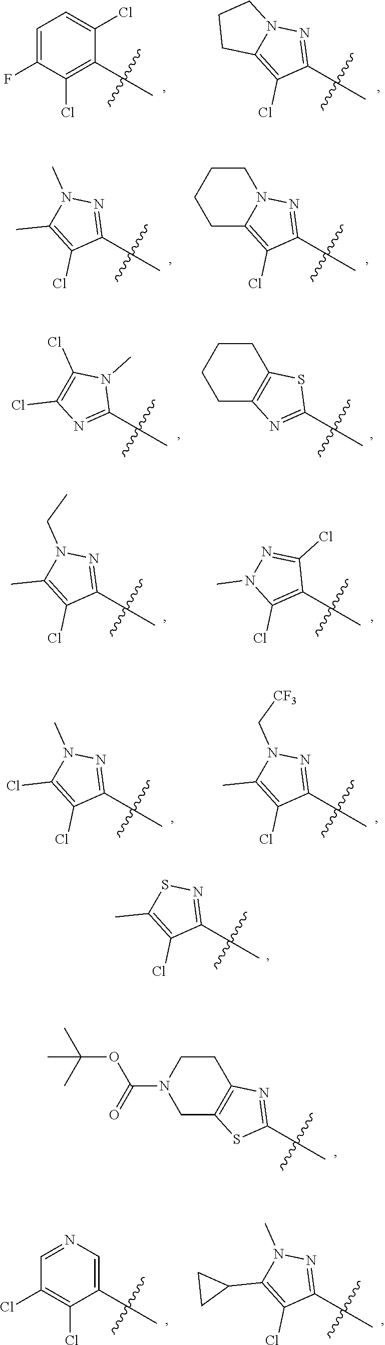

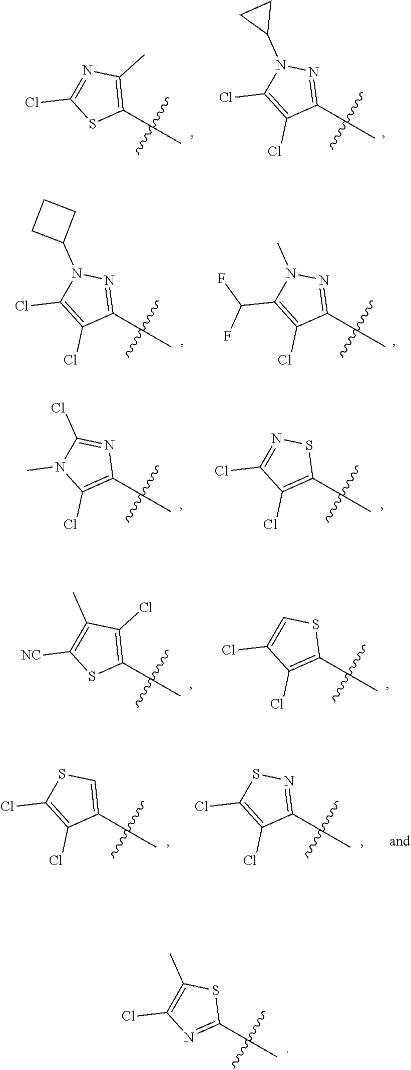

48. The compound, tautomer, solvate, stereoisomer, or pharmaceutically acceptable salt of claim 1, wherein Ring A substituted with m groups of R^{a} is selected from:

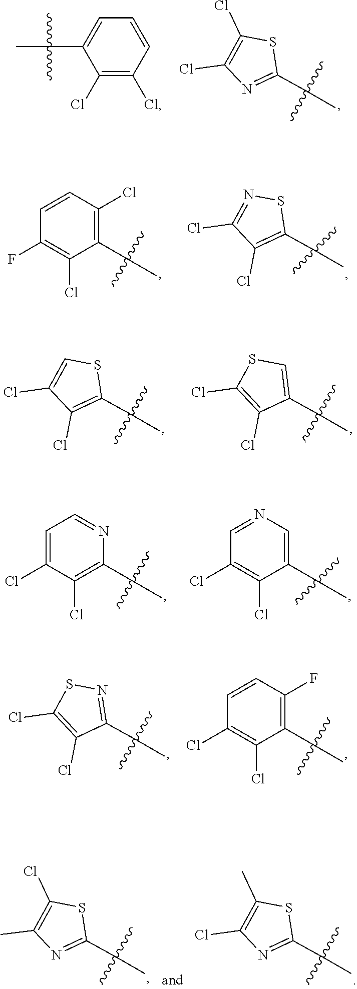

49. The compound, tautomer, solvate, stereoisomer, or pharmaceutically acceptable salt of claim 1, wherein Ring B substituted with n groups of R^{b} is: 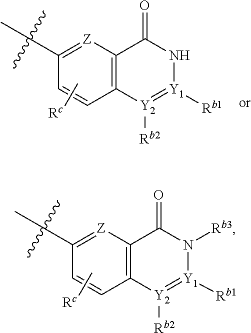
wherein Z is selected from C and N, Y₁ and Y₂ are independently selected from C and N, R^{b1} and R^{b2} are independently selected from H, F, Cl, Br, methyl, CN, CF₃, CF₂H, ethyl, cyclopropyl, and OCH₃, wherein R^{b1} and R^{b2} are absent when connected to N in Ring B, and R^{b3} is selected from H, methyl, ethyl, 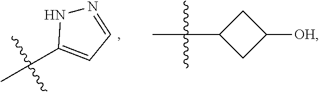 [SMILES: *c1ccn[nH]1 ; *C1CC(O)C1]
and 5-membered heteroaryl, wherein the 5-membered heteroaryl of R^{b3} contains 2 to 3 heteroatoms selected from N and S and the 5-membered heteroaryl is optionally substituted with 1-2 groups selected from halogen and C₁ to C₄ alkyl, and R^{c} is selected from H, halogen, CN, and methyl.

50. The compound, tautomer, solvate, stereoisomer, or pharmaceutically acceptable salt of claim 1, wherein Ring B substituted with n groups of R^{b} is selected from: 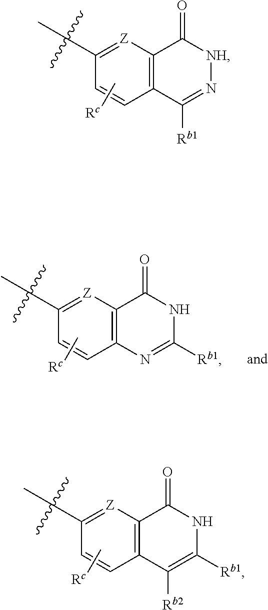
wherein Z is selected from C and N, R^{b1} is selected from H, F, Cl, Br, methyl, CN, CF₃, CF₂H, ethyl, cyclopropyl, and OCH₃, R^{b2} is selected from H, F, Cl, Br, methyl, CN, CF₃, CF₂H, ethyl, cyclopropyl, and OCH₃, and R^{c} is selected from H, halogen, CN, and methyl.

51. The compound, tautomer, solvate, stereoisomer, or pharmaceutically acceptable salt of claim 1, wherein Ring B substituted with n groups of R^{b} is: 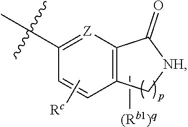
wherein Z is selected from C and N, R^{b1} is selected from F, Cl, Br, methyl, CN, CF₃, CF₂H, ethyl, cyclopropyl, and OCH₃, R^{c} is selected from H, halogen, CN, and methyl, p is 1, 2, or 3, and q is 0, 1, or 2.

52. The compound, tautomer, solvate, stereoisomer, or pharmaceutically acceptable salt of claim 1, wherein Ring B substituted with n groups of R^{b} is selected from: 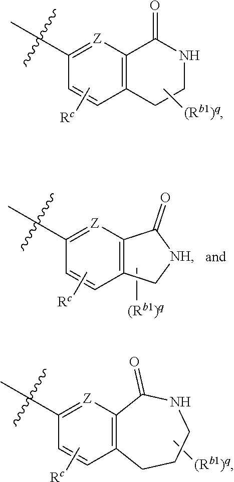
wherein Z is selected from C and N, R^{b1} is selected from F, Cl, Br, methyl, CN, CF₃, CF₂H, ethyl, cyclopropyl, and OCH₃, R^{c} is selected from H, halogen, CN, and methyl, and q is 0, 1 or 2.

53. The compound, tautomer, solvate, stereoisomer, or pharmaceutically acceptable salt of claim 1, wherein Ring B substituted with n groups of R^{b} is: 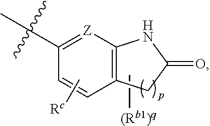
wherein Z is selected from C and N, R^{b1} selected from F, Cl, Br, methyl, CN, CF₃, CF₂H, ethyl, cyclopropyl, and OCH₃, R^{c} is H, halogen, CN, or methyl, p is 1, 2 or 3, q is 0, 1, or 2.

54. The compound, tautomer, solvate, stereoisomer, or pharmaceutically acceptable salt of claim 1, wherein Ring B substituted with n groups of R^{b} is selected from: 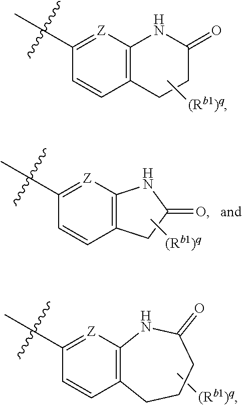
wherein Z is selected from C and N, R^{b1} selected from F, Cl, Br, methyl, CN, CF₃, CF₂H, ethyl, cyclopropyl, and OCH₃, and q is 0, 1 or 2.

55. The compound, tautomer, solvate, stereoisomer, or pharmaceutically acceptable salt of claim 1, wherein Ring B substituted with n groups of R^{b} is 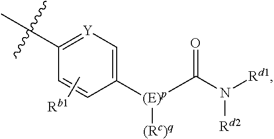
wherein Y is selected from C and N, E is selected from C, N, and O, R^{d1} and R^{d2} are independently selected from H, methyl, ethyl, and cyclopropyl, R^{c} is selected from F, Cl, Br, methyl, CN, CF₃, CF₂H, ethyl, cyclopropyl, and OCH₃, R^{b1} is selected from H, halogen, CN, and methyl, p is 1 or 2, and q is 0, 1, or 2.

56. The compound, tautomer, solvate, stereoisomer, or pharmaceutically acceptable salt of claim 1, wherein Ring B substituted with n groups of R^{b} is 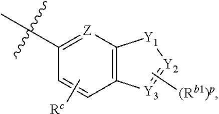
wherein Z is selected from C and N, Y₁, Y₂, and Y₃ are independently selected from C, N, S, and O, R^{b1} is selected from F, Cl, Br, methyl, CN, CF₃, CF₂H, ethyl, cyclopropyl, CONH₂, OCH₃, CH₂CONH₂, 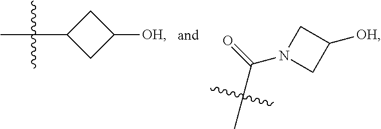 [SMILES: *C1CC(O)C1 ; "and" ; *C(=O)N1CC(O)C1]
R^{c} is selected from H, halogen, CN, and methyl, and p is 0, 1, or 2.

57. The compound, tautomer, solvate, stereoisomer, or pharmaceutically acceptable salt of claim 1, wherein Ring B substituted with n groups of R^{b} is selected from

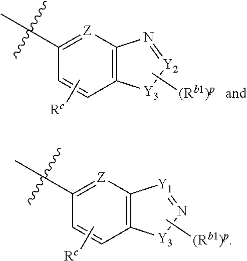

58. The compound, tautomer, solvate, stereoisomer, or pharmaceutically acceptable salt of claim 1, wherein Ring B substituted with n groups of R^{b} is 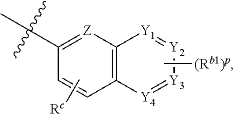
Z is selected from C and N, Y₁, Y₂, Y₃, and Y₄ are independently selected from C and N, R^{b1} is selected from F, Cl, Br, methyl, CN, CF₃, CF₂H, ethyl, cyclopropyl, CONH₂, and OCH₃, R^{c} is H, halogen, CN, or methyl, and p is 0, 1, or 2.

59. The compound, tautomer, solvate, stereoisomer, or pharmaceutically acceptable salt of claim 1, wherein Ring B is selected from: 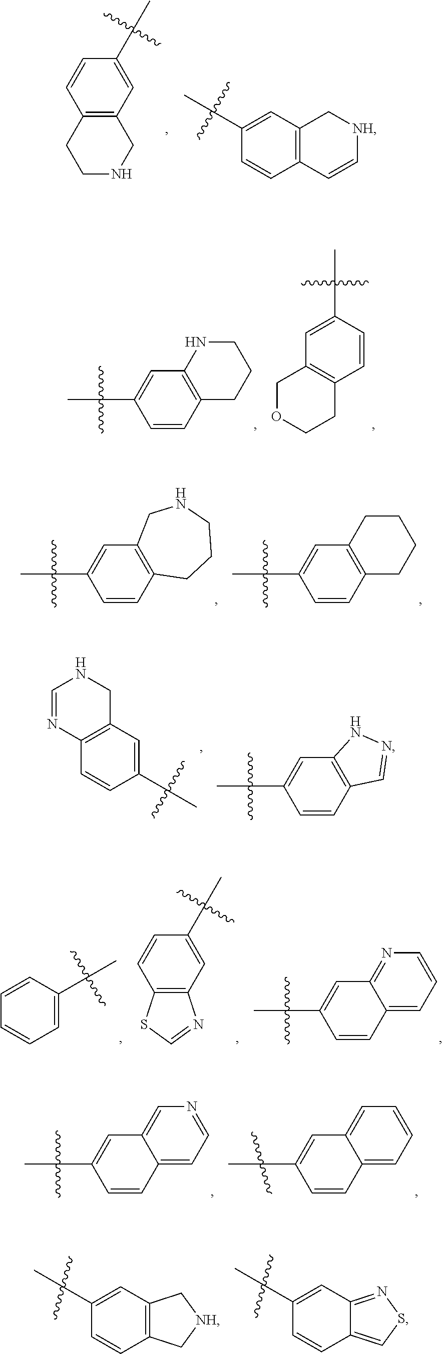 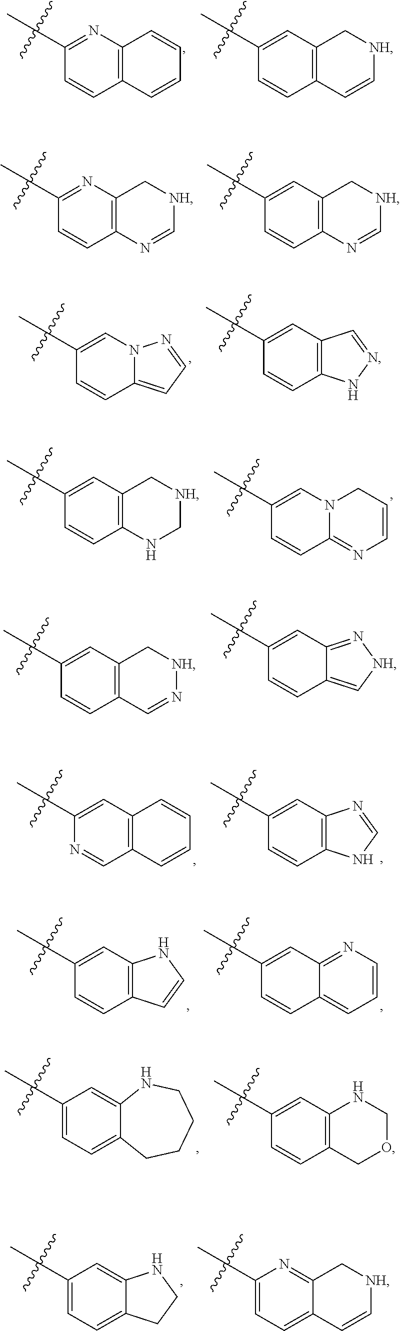 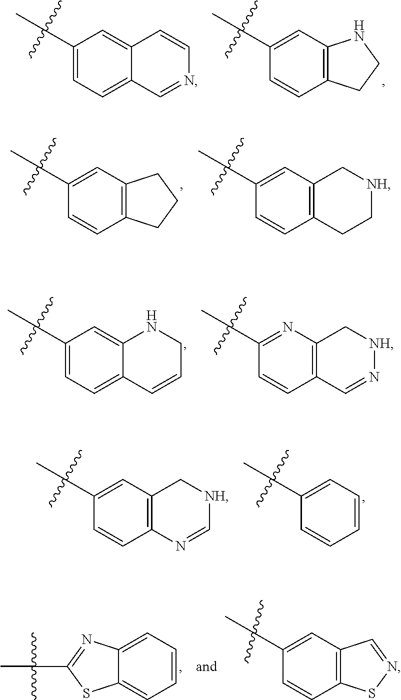
wherein Ring B is substituted with n groups of R^{b}.

60. The compound, tautomer, solvate, stereoisomer, or pharmaceutically acceptable salt of claim 1, wherein Ring B substituted with n groups of R^{b} is selected from:

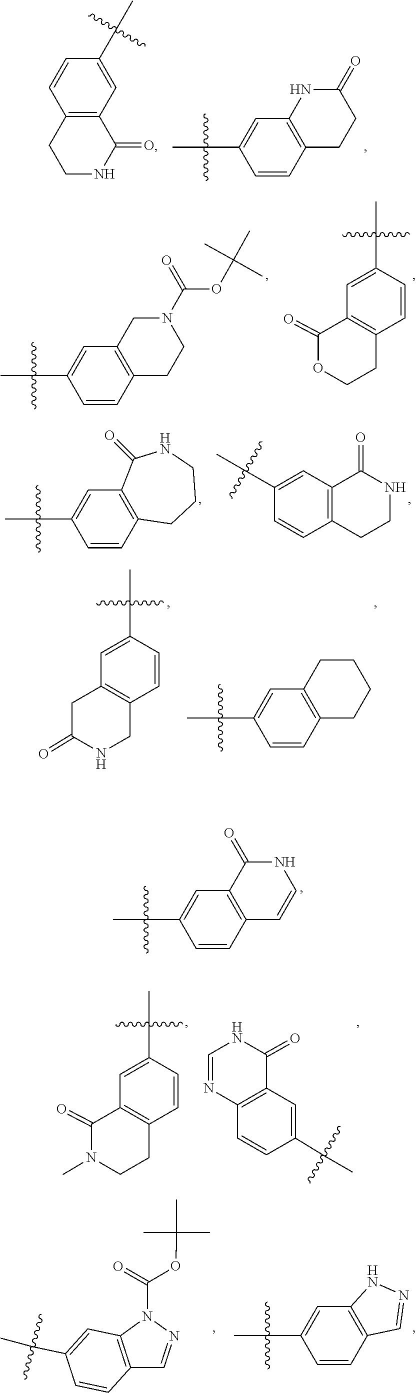

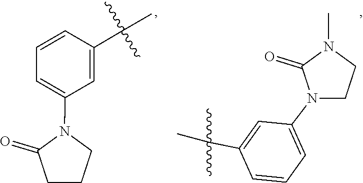 [SMILES: *c1cccc(N2CCCC2=O)c1 ; *c1cccc(N2CCN(C)C2=O)c1]

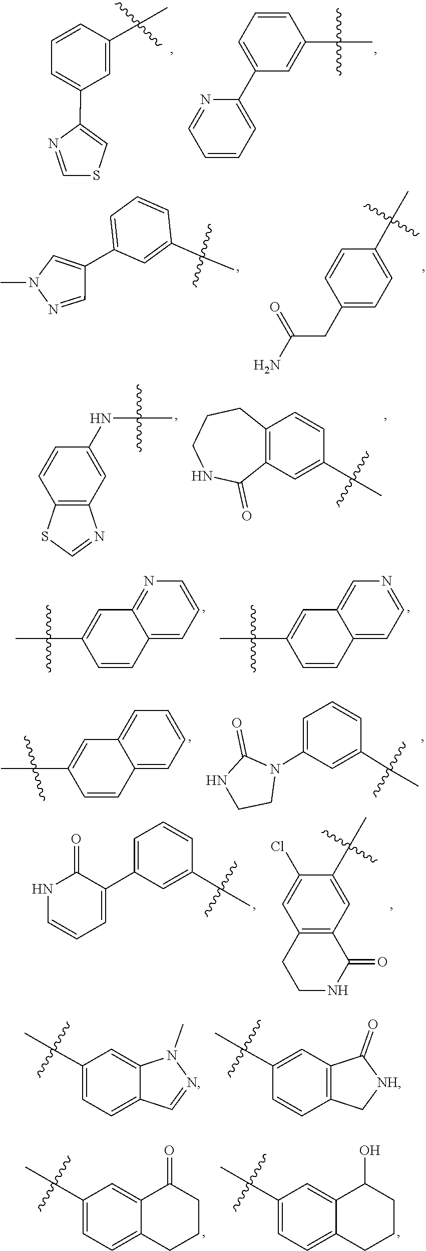

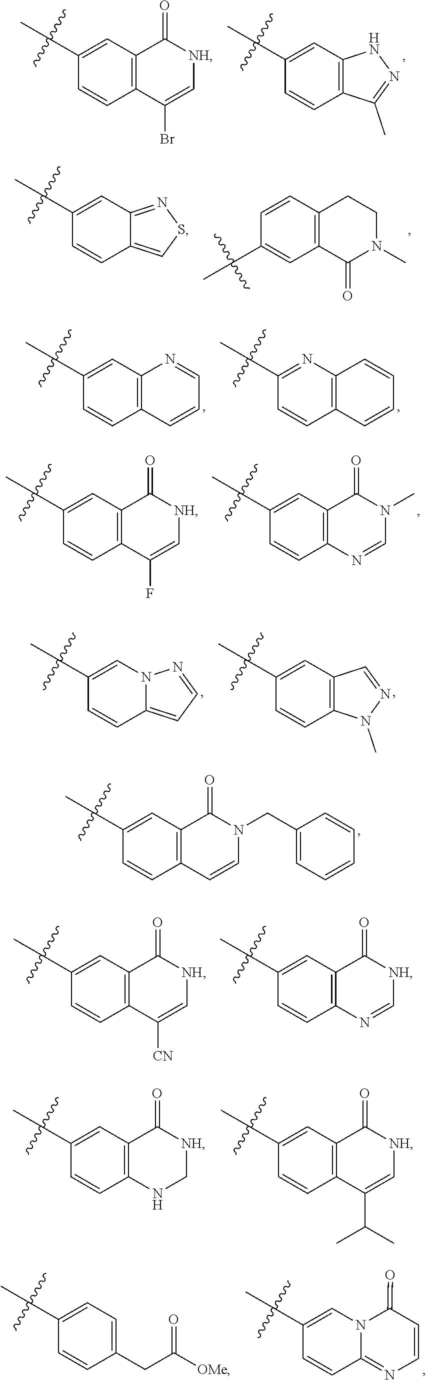

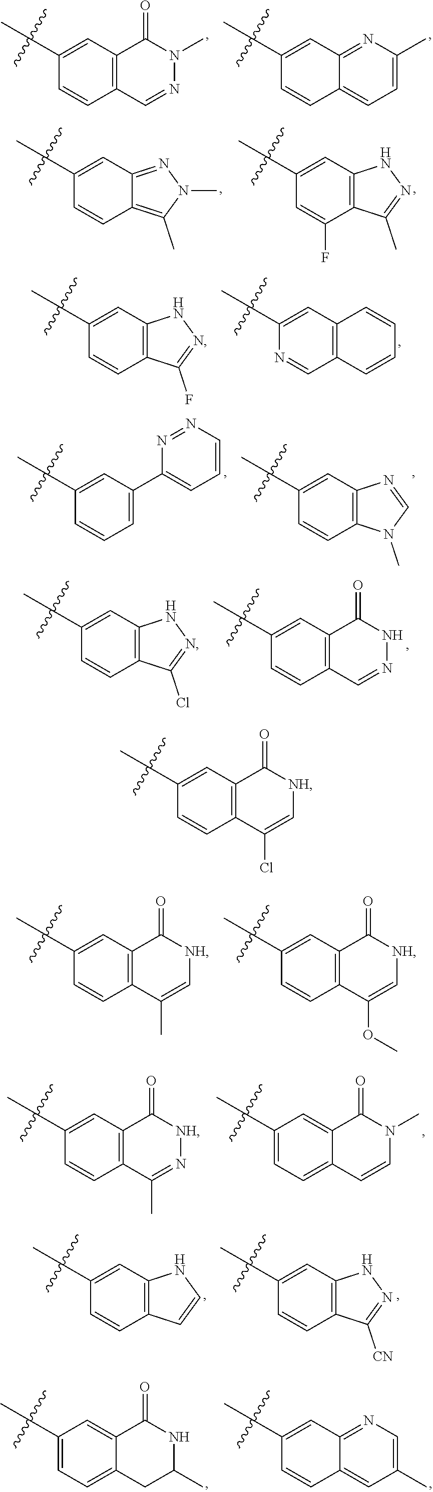

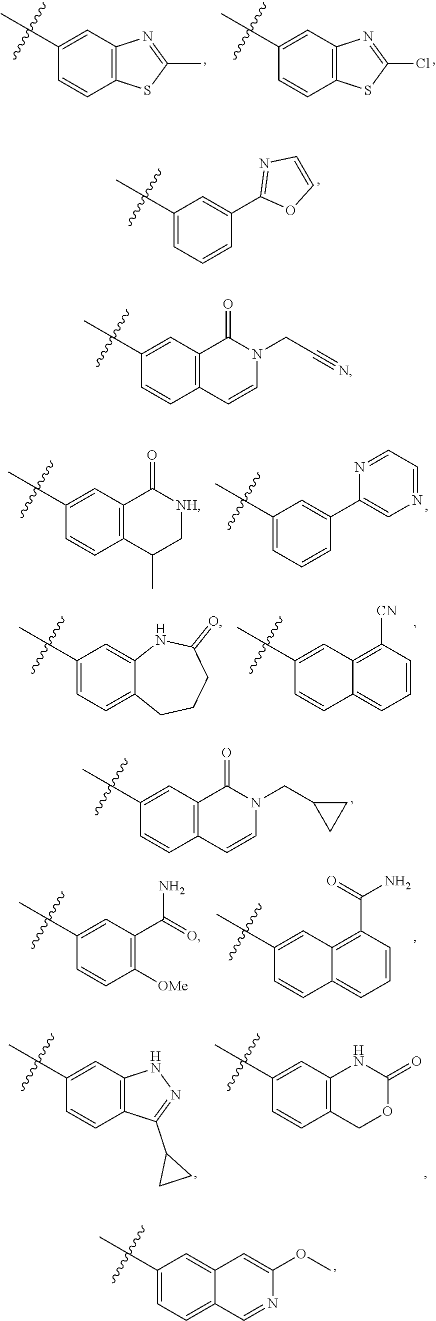

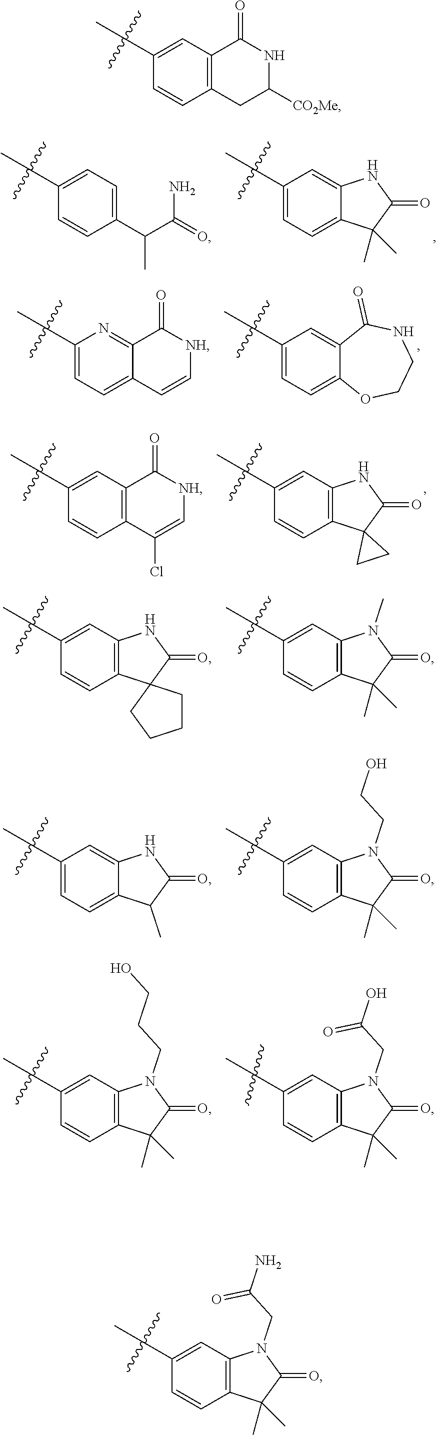

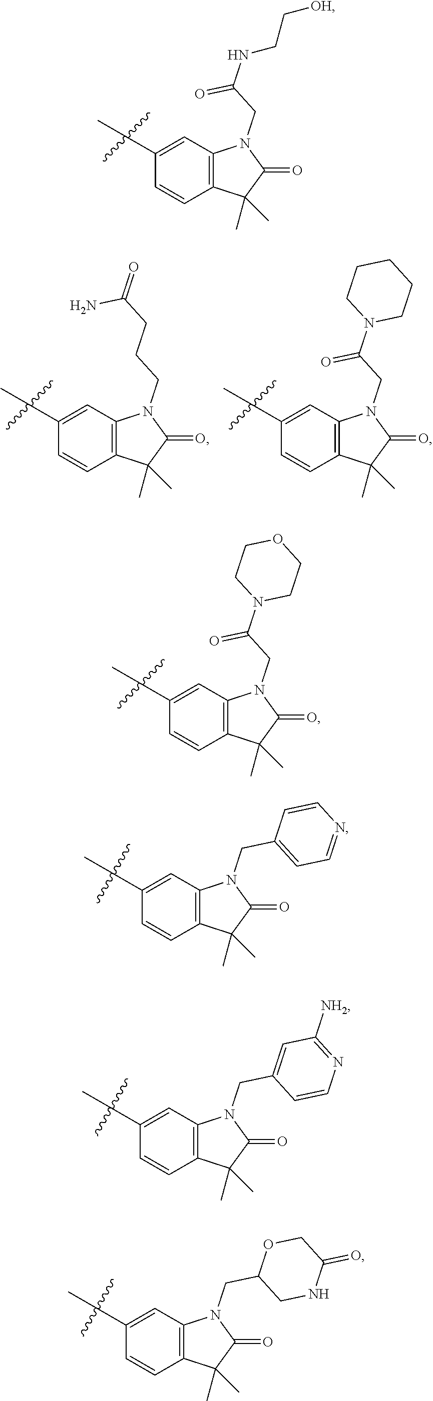

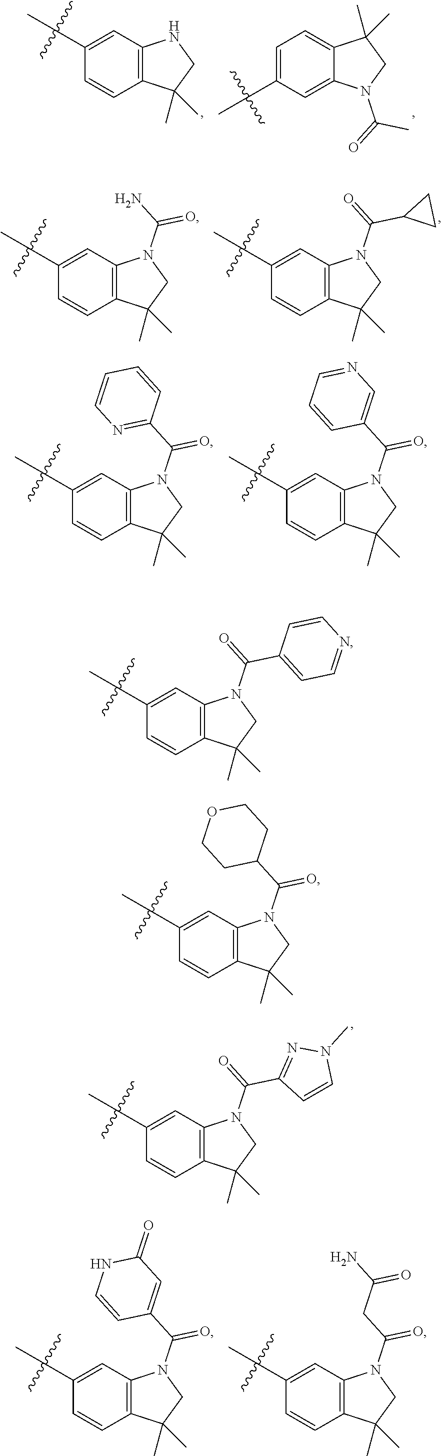

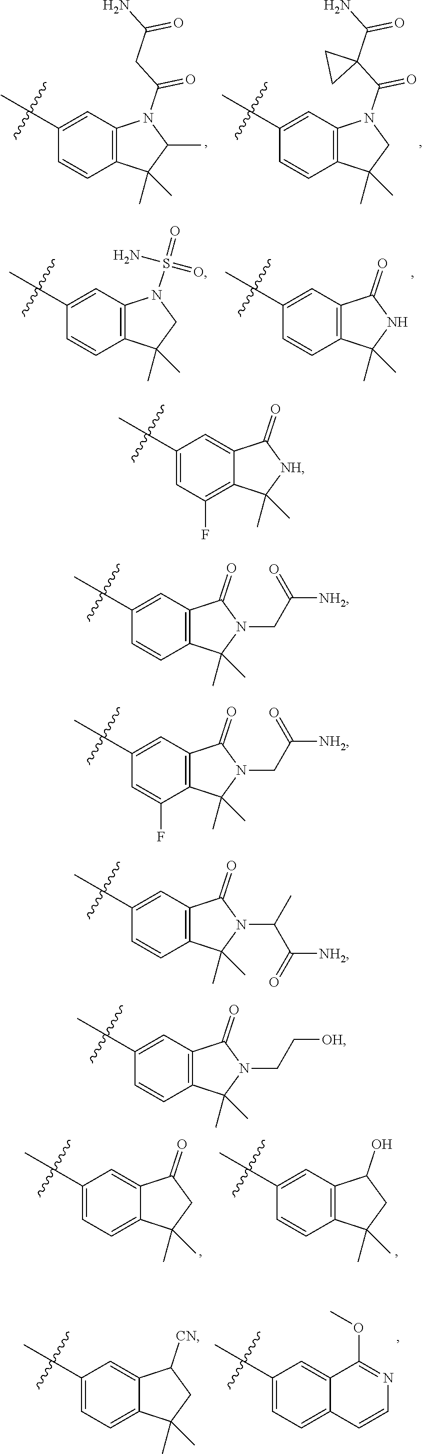

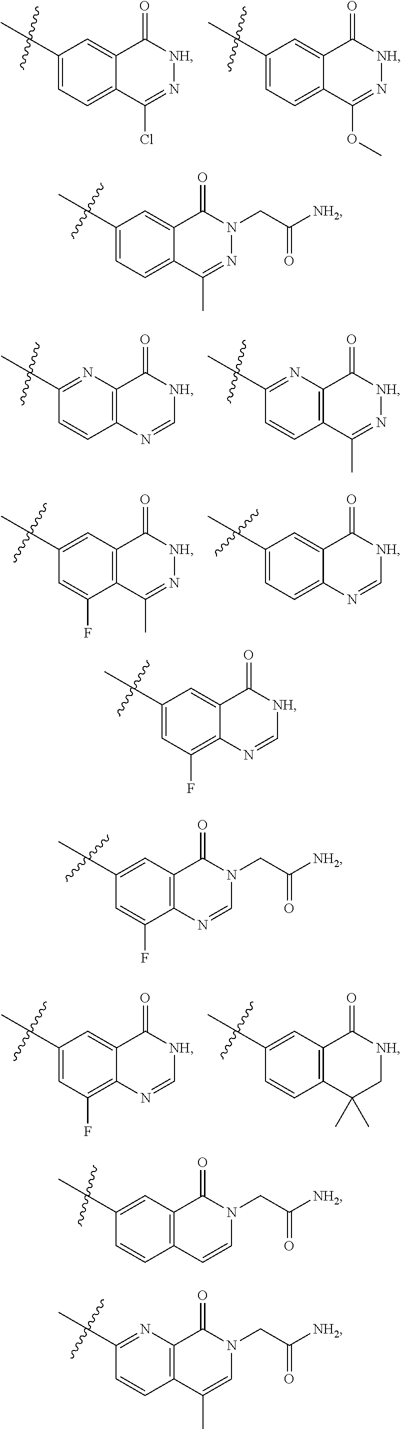

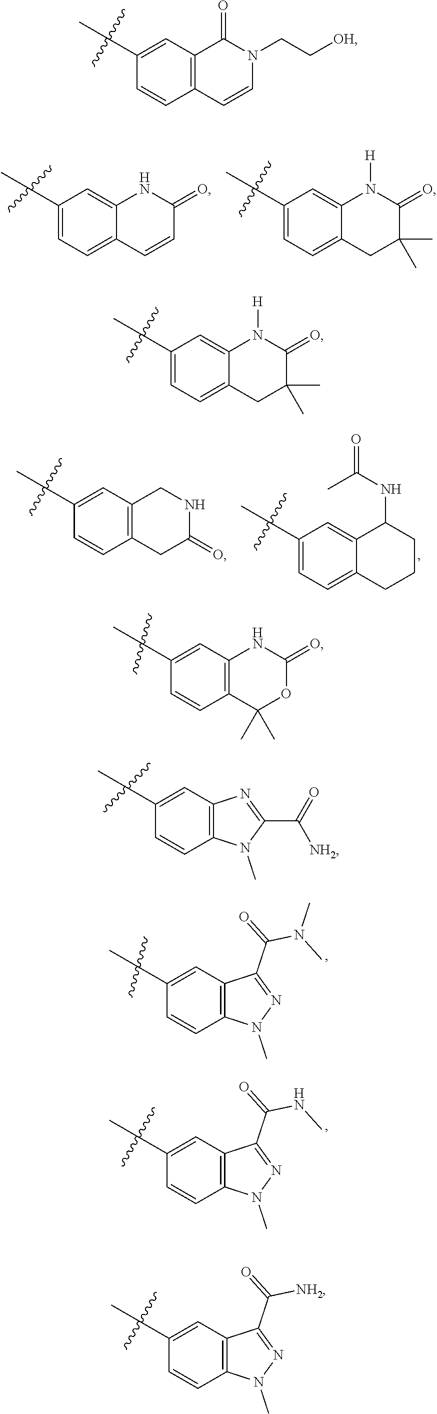

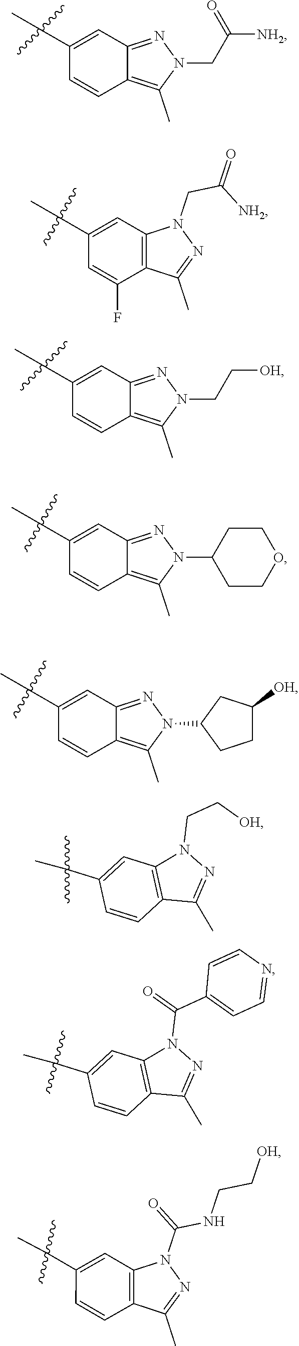

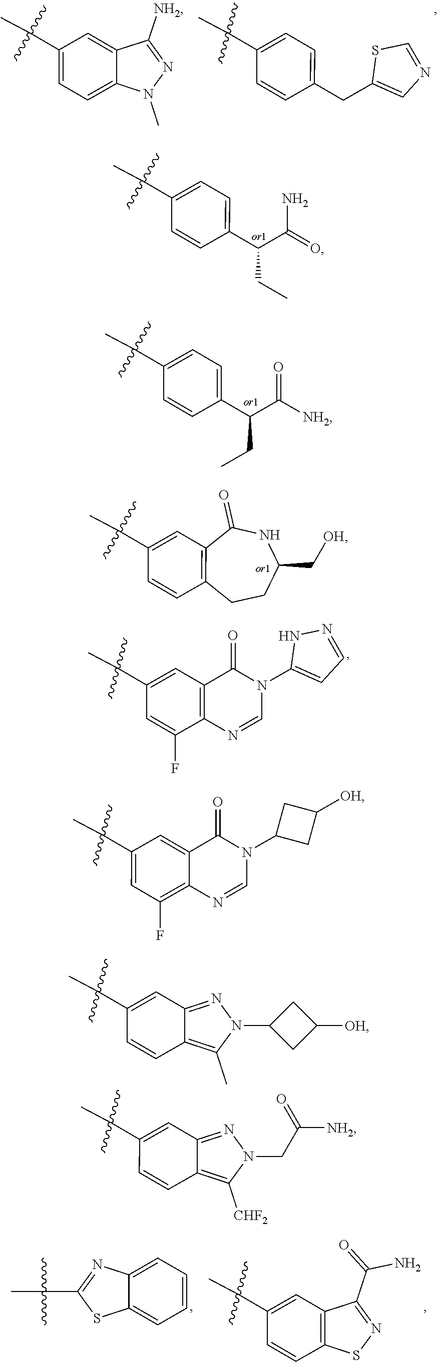

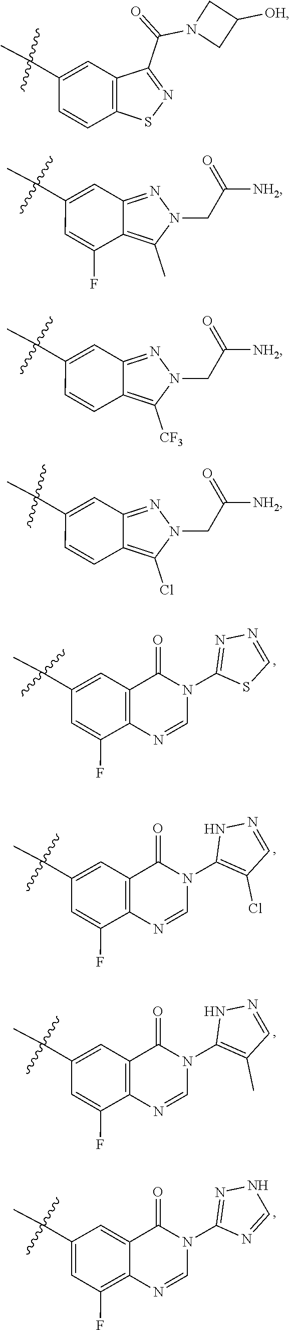

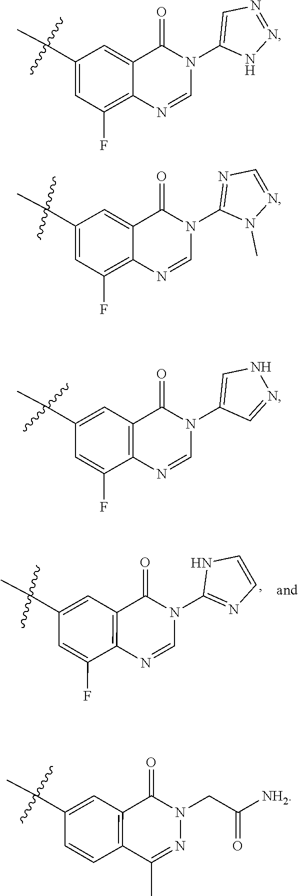 [SMILES: *c1cc(F)c2ncn(-c3cnn[nH]3)c(=O)c2c1 ; *c1cc(F)c2ncn(-c3ncnn3C)c(=O)c2c1 ; *c1cc(F)c2ncn(-c3cn[nH]c3)c(=O)c2c1 ; *c1cc(F)c2ncn(-c3ncc[nH]3)c(=O)c2c1 ; *c1ccc2c(C)nn(CC(N)=O)c(=O)c2c1]

61. The compound, tautomer, solvate, stereoisomer, or pharmaceutically acceptable salt of claim 1, wherein Ring B substituted with n groups of R^{b} is selected from: 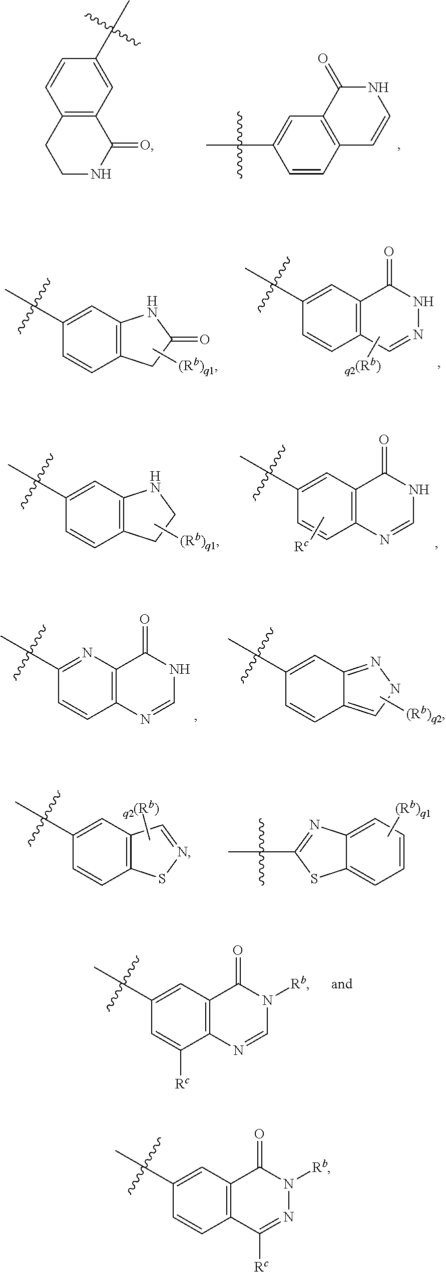
wherein q1 is an integer selected from 0, 1, 2, and 3, q2 is an integer selected from 0, 1, and 2, and R^{c} is selected from F, Cl, Br and Me.

62. The compound, tautomer, solvate, stereoisomer, or pharmaceutically acceptable salt of claim 1, wherein R^{a} is selected from C₃-C₅ cycloalkyl, —O(C₁-C₂ alkyl), COOH, CN, CONH₂, —C(═O)NH(C₁-C₂ alkyl), —C(═O)N(C₁-C₂ alkyl)₂, —C(═O)O(C₁-C₂ alkyl), halogen, and C₁-C₂ alkyl optionally substituted with 1 to 3 groups selected from halogen.

63. The compound, tautomer, solvate, stereoisomer, or pharmaceutically acceptable salt of claim 1, wherein R^{a} is selected from CH₃, CH₂CH₃, CH₂CF₃, F, Cl, Br, C₃-C₄ cycloalkyl, COOH, CN, CONH₂, —C(═O)NHCH₃, —C(═O)NCH₃, —OC(═O)N(CH₃)₃, and OCH₃.

64. The compound, tautomer, solvate, stereoisomer, or pharmaceutically acceptable salt of claim 1, wherein R^{b} is selected from ═O, OH, halogen, CN, —C(═O)NH(C₁-C₄ alkyl), —C(═O)O(C₁-C₄ alkyl), —C(═O)NH₂,
C₃-C₄ cycloalkyl optionally substituted with OH,
O(C₁-C₃ alkyl), —C(═O)(C₁-C₂ alkyl), —C(═O)(C₃-C₆ cycloalkyl), —C(═O)(4- to 6-membered heterocyclyl optionally substituted with ═O), —C(═O)(5- to 6-membered heteroaryl optionally substituted with C₁-C₂ alkyl or NH₂), —C(═O)(CH₂)pC(═O)NH₂, —NHC(═O)(C₁-C₂ alkyl), —C(═O)N(C₁-C₂ alkyl)₂, —C(═O)NH(CH₂)pOH, —S(═O)₂NH₂, 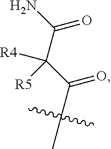
C₁-C₃ alkyl optionally substituted with 1 to 3 groups selected from halogen, NH₂, OH, OCH₃, —C(═O)NH(C₁-C₄ alkyl), —C(═O)O(C₁-C₄ alkyl), —C(═O)N(C₁-C₄ alkyl)₂, —COOH, —C(═O)NH₂, CN, C₃-C₄ cycloalkyl, phenyl, —C(═O)(C₁-C₂ alkyl), —C(═O)(C₃-C₆ cycloalkyl), —C(═O)(5- to 6-membered heterocyclyl), —C(═O)(5- to 6-membered heteroaryl optionally substituted with C₁-C₂ alkyl), C(═O)NH(CH₂)pOH, and 5- to 6-membered heteroaryl,
5- to 6-membered heteroaryl optionally substituted with 1 to 2 groups selected from C₁-C₂ alkyl, and
5- to 6-membered heterocyclyl optionally substituted with 1 to 2 groups selected from ═O and C₁-C₂ alkyl, or
two R^{b} attached to the same position on Ring B join to form a C₃-C₅ cycloalkyl,
wherein p is an integer selected from 1 and 2, and
wherein R₄ and R₅ are each independently selected from H and C₁ to C₃ alkyl or R₄ and R₅ join to form a 3 to 5-membered cycloalkyl.

65. The compound, tautomer, solvate, stereoisomer, or pharmaceutically acceptable salt of claim 1, wherein R^{b} is selected from ═O, 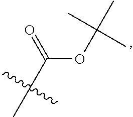 [SMILES: *C(=O)OC(C)(C)C]
CH₃, CHF₂, CF₃, OH, F, Cl, Br, CN, CH(CH₃)₂, CH₂CN, OCH₃, —C(═O)NH₂, —C(═O)OCH₃, 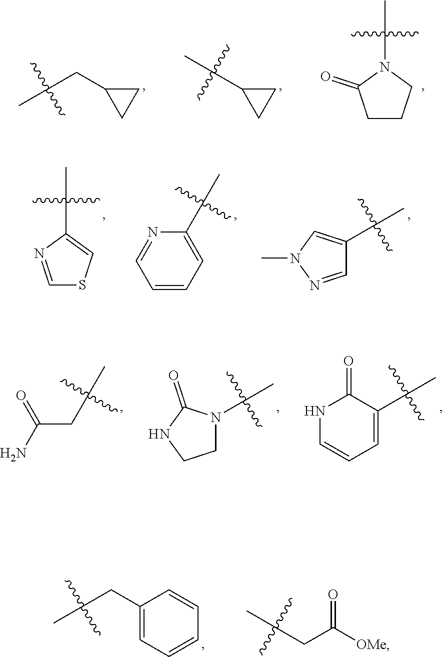
CH₂CH₂OH, CH₂CH₂CH₂OH, CH₂C(═O)OH, CH₂C(═O)NH₂, CH₂C(═O)NHCH₂CH₂OH, CH₂CH₂CH₂C(═O)NH₂, 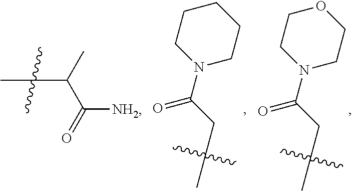
—C(═O)CH₃, 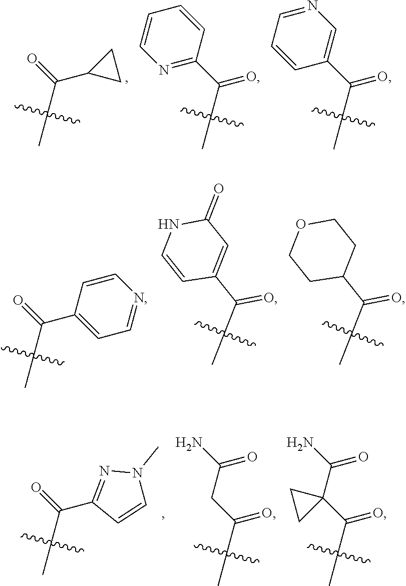
—NHC(═O)CH₃, —C(═O)N(CH₃)₂, —C(═O)NCH₃, NH₂, —C(═O)NHCH₂CH₂OH, 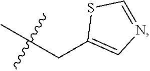 [SMILES: *Cc1cncs1]
CH₂OH, 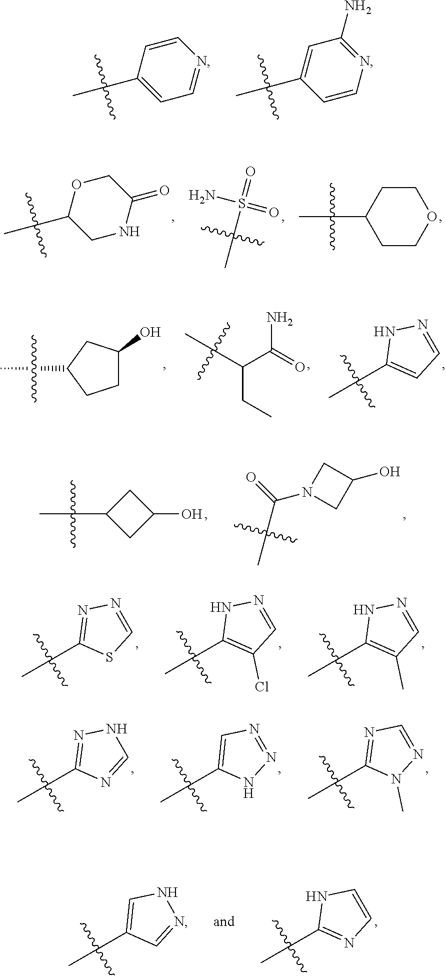
or two R^{b} attached to the same position on B join to form a 3-membered or 5-membered cycloalkyl.

66. The compound, tautomer, solvate, stereoisomer, or pharmaceutically acceptable salt of claim 1, wherein L is selected from 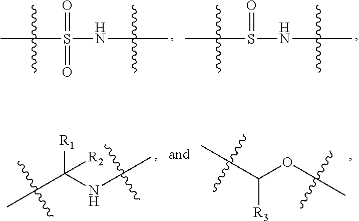
wherein
R₁ is H, D or C₁-C₄ alkyl, and R₂ is selected from CN, C₁-C₄ alkenyl, C₃-C₅ cycloalkyl, 5 to 6-membered heteroaryl,
phenyl optionally substituted with 1 to 2 groups selected from C₁-C₃ alkyl and halogen, and
C₁-C₄ alkyl optionally substituted with 1 to 3 groups selected from halo, OH, and phenyl,
or
R₁ and R₂ join to form a 3-4 membered heterocyclyl; and
R₃ is C₁-C₄ alkyl optionally substituted with 1 to 3 groups selected from halogen and OH.

67. The compound, tautomer, solvate, stereoisomer, or pharmaceutically acceptable salt of claim 1, wherein L is selected from

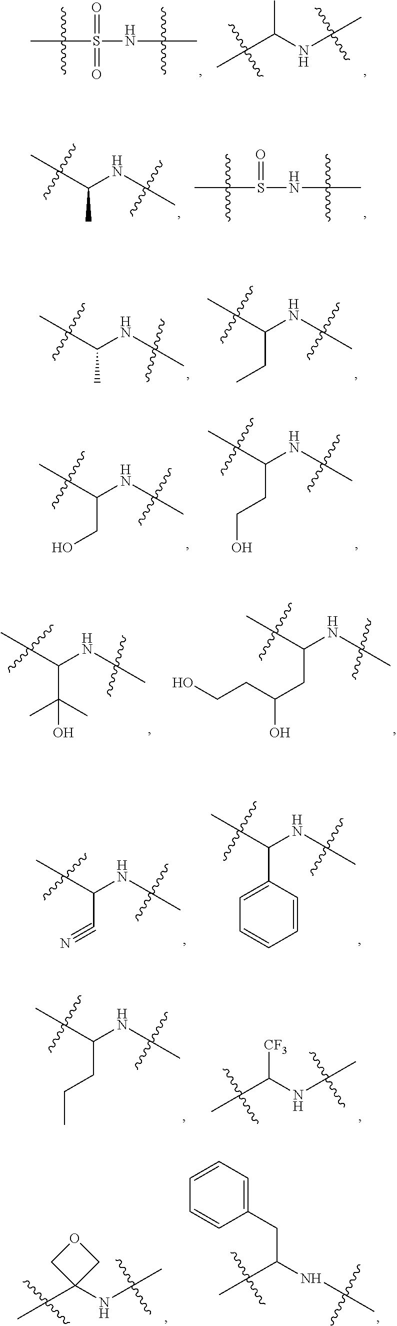

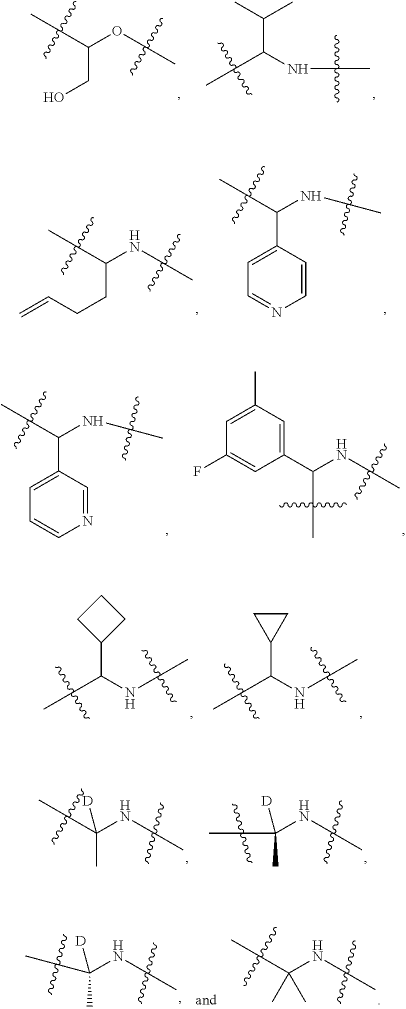

68. The compound, tautomer, solvate, stereoisomer, or pharmaceutically acceptable salt of claim 1, wherein L is selected from

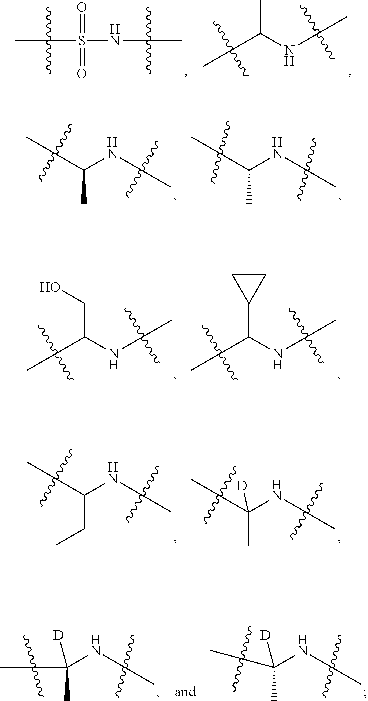

69. The compound according to claim 1, wherein the compound is selected from the compounds below, 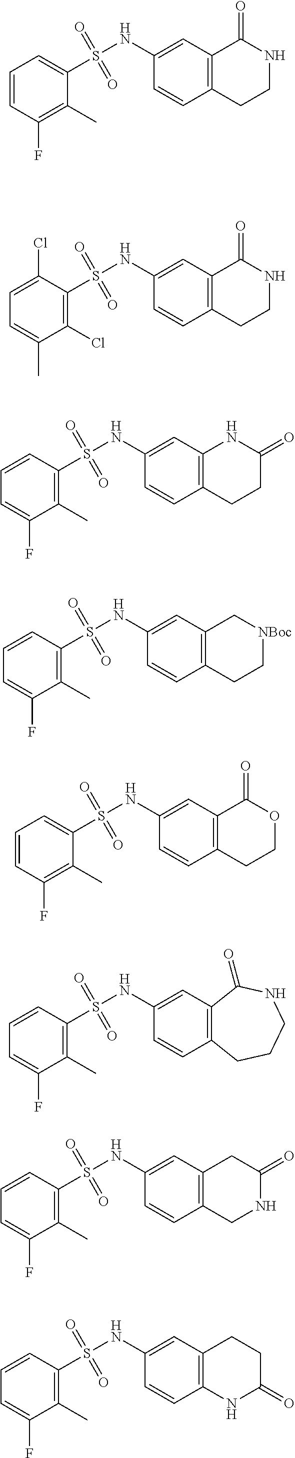 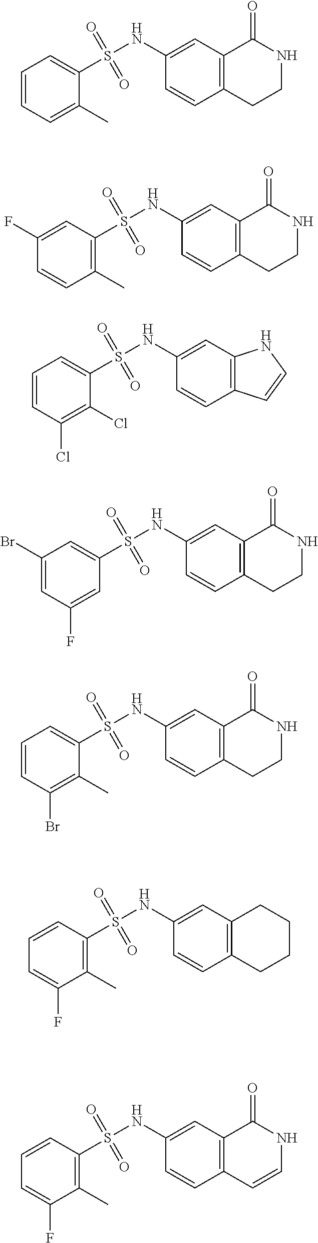 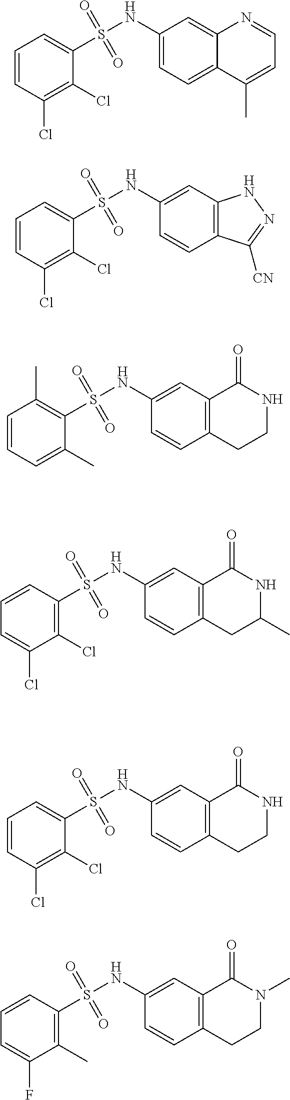 [SMILES: Cc1ccnc2cc(NS(=O)(=O)c3cccc(Cl)c3Cl)ccc12 ; N#Cc1n[nH]c2cc(NS(=O)(=O)c3cccc(Cl)c3Cl)ccc12 ; Cc1cccc(C)c1S(=O)(=O)Nc1ccc2c(c1)C(=O)NCC2 ; CC1Cc2ccc(NS(=O)(=O)c3cccc(Cl)c3Cl)cc2C(=O)N1 ; O=C1NCCc2ccc(NS(=O)(=O)c3cccc(Cl)c3Cl)cc21 ; Cc1c(F)cccc1S(=O)(=O)Nc1ccc2c(c1)C(=O)N(C)CC2] 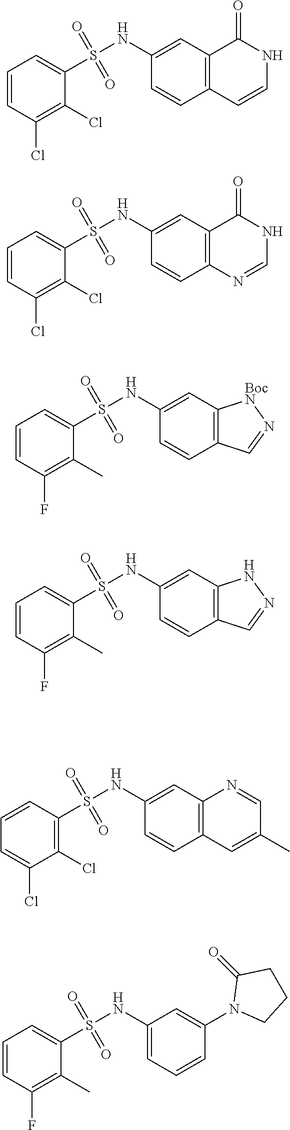 [SMILES: O=c1[nH]ccc2ccc(NS(=O)(=O)c3cccc(Cl)c3Cl)cc12 ; O=c1[nH]cnc2ccc(NS(=O)(=O)c3cccc(Cl)c3Cl)cc12 ; Cc1c(F)cccc1S(=O)(=O)Nc1ccc2cnn(C(=O)OC(C)(C)C)c2c1 ; Cc1c(F)cccc1S(=O)(=O)Nc1ccc2cn[nH]c2c1 ; Cc1cnc2cc(NS(=O)(=O)c3cccc(Cl)c3Cl)ccc2c1 ; Cc1c(F)cccc1S(=O)(=O)Nc1cccc(N2CCCC2=O)c1] 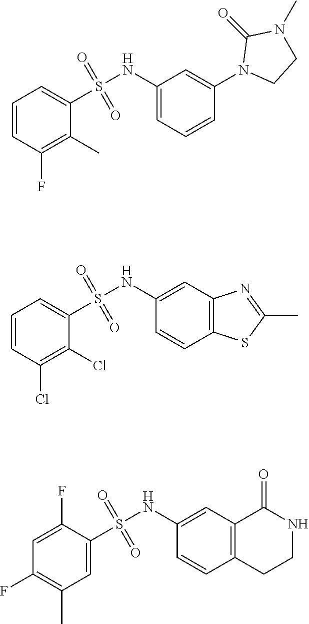 [SMILES: Cc1c(F)cccc1S(=O)(=O)Nc1cccc(N2CCN(C)C2=O)c1 ; Cc1nc2cc(NS(=O)(=O)c3cccc(Cl)c3Cl)ccc2s1 ; Cc1cc(S(=O)(=O)Nc2ccc3c(c2)C(=O)NCC3)c(F)cc1F] 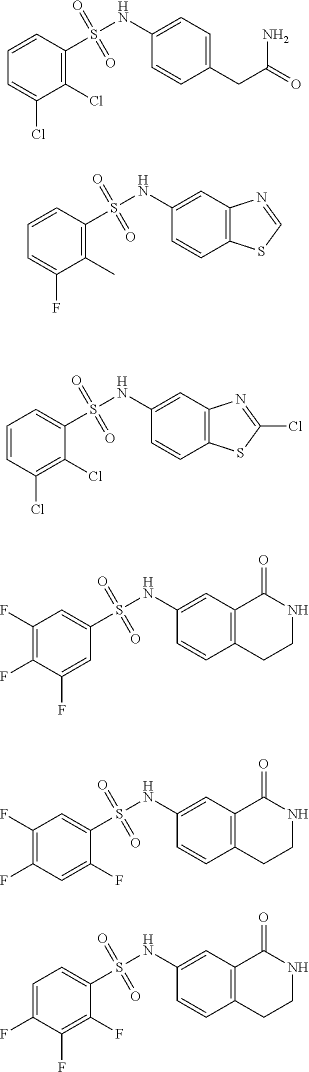 [SMILES: NC(=O)Cc1ccc(NS(=O)(=O)c2cccc(Cl)c2Cl)cc1 ; Cc1c(F)cccc1S(=O)(=O)Nc1ccc2scnc2c1 ; O=S(=O)(Nc1ccc2sc(Cl)nc2c1)c1cccc(Cl)c1Cl ; O=C1NCCc2ccc(NS(=O)(=O)c3cc(F)c(F)c(F)c3)cc21 ; O=C1NCCc2ccc(NS(=O)(=O)c3cc(F)c(F)cc3F)cc21 ; O=C1NCCc2ccc(NS(=O)(=O)c3ccc(F)c(F)c3F)cc21] 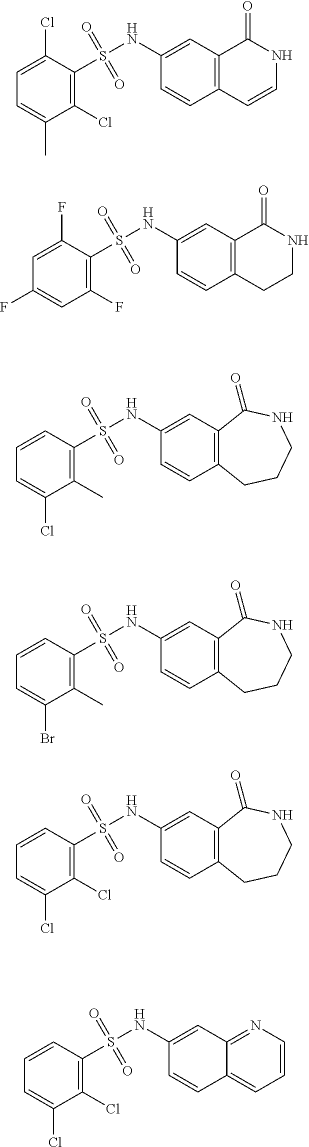 [SMILES: Cc1ccc(Cl)c(S(=O)(=O)Nc2ccc3cc[nH]c(=O)c3c2)c1Cl ; O=C1NCCc2ccc(NS(=O)(=O)c3c(F)cc(F)cc3F)cc21 ; Cc1c(Cl)cccc1S(=O)(=O)Nc1ccc2c(c1)C(=O)NCCC2 ; Cc1c(Br)cccc1S(=O)(=O)Nc1ccc2c(c1)C(=O)NCCC2 ; O=C1NCCCc2ccc(NS(=O)(=O)c3cccc(Cl)c3Cl)cc21 ; O=S(=O)(Nc1ccc2cccnc2c1)c1cccc(Cl)c1Cl] 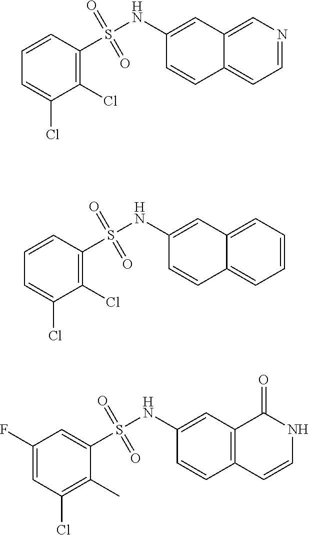 [SMILES: O=S(=O)(Nc1ccc2ccncc2c1)c1cccc(Cl)c1Cl ; O=S(=O)(Nc1ccc2ccccc2c1)c1cccc(Cl)c1Cl ; Cc1c(Cl)cc(F)cc1S(=O)(=O)Nc1ccc2cc[nH]c(=O)c2c1] 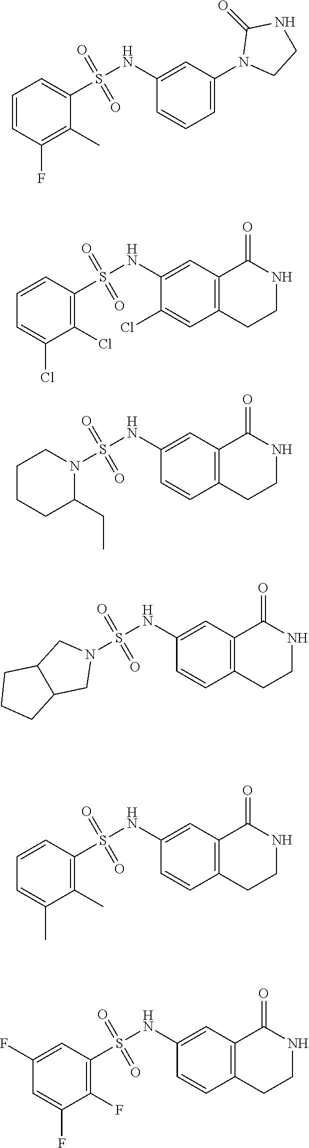 [SMILES: Cc1c(F)cccc1S(=O)(=O)Nc1cccc(N2CCNC2=O)c1 ; O=C1NCCc2cc(Cl)c(NS(=O)(=O)c3cccc(Cl)c3Cl)cc21 ; CCC1CCCCN1S(=O)(=O)Nc1ccc2c(c1)C(=O)NCC2 ; O=C1NCCc2ccc(NS(=O)(=O)N3CC4CCCC4C3)cc21 ; Cc1cccc(S(=O)(=O)Nc2ccc3c(c2)C(=O)NCC3)c1C ; O=C1NCCc2ccc(NS(=O)(=O)c3cc(F)cc(F)c3F)cc21] 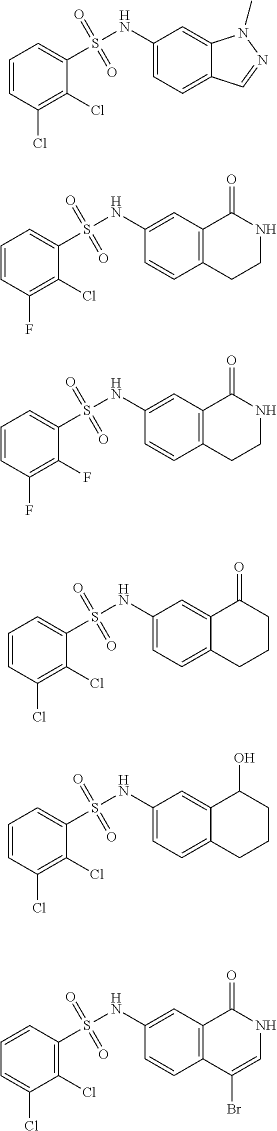 [SMILES: Cn1ncc2ccc(NS(=O)(=O)c3cccc(Cl)c3Cl)cc21 ; O=C1NCCc2ccc(NS(=O)(=O)c3cccc(F)c3Cl)cc21 ; O=C1NCCc2ccc(NS(=O)(=O)c3cccc(F)c3F)cc21 ; O=C1CCCc2ccc(NS(=O)(=O)c3cccc(Cl)c3Cl)cc21 ; O=S(=O)(Nc1ccc2c(c1)C(O)CCC2)c1cccc(Cl)c1Cl ; O=c1[nH]cc(Br)c2ccc(NS(=O)(=O)c3cccc(Cl)c3Cl)cc12] 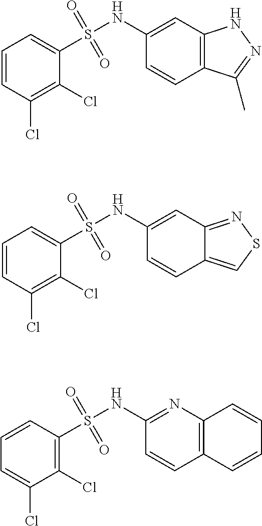 [SMILES: Cc1n[nH]c2cc(NS(=O)(=O)c3cccc(Cl)c3Cl)ccc12 ; O=S(=O)(Nc1ccc2csnc2c1)c1cccc(Cl)c1Cl ; O=S(=O)(Nc1ccc2ccccc2n1)c1cccc(Cl)c1Cl] 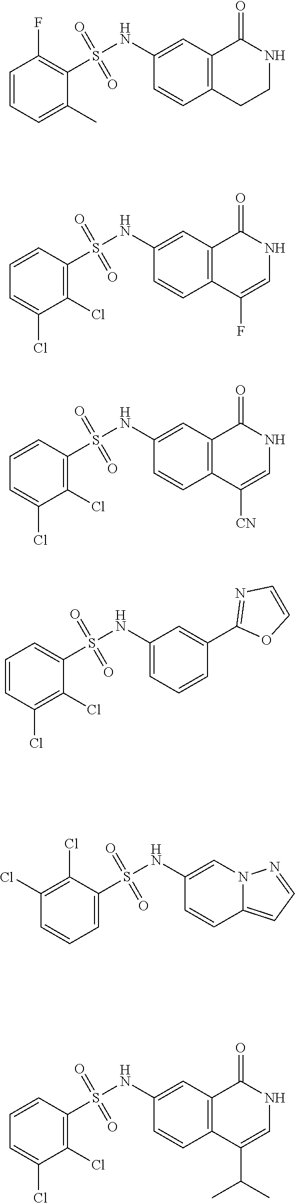 [SMILES: Cc1cccc(F)c1S(=O)(=O)Nc1ccc2c(c1)C(=O)NCC2 ; O=c1[nH]cc(F)c2ccc(NS(=O)(=O)c3cccc(Cl)c3Cl)cc12 ; N#Cc1c[nH]c(=O)c2cc(NS(=O)(=O)c3cccc(Cl)c3Cl)ccc12 ; O=S(=O)(Nc1cccc(-c2ncco2)c1)c1cccc(Cl)c1Cl ; O=S(=O)(Nc1ccc2ccnn2c1)c1cccc(Cl)c1Cl ; CC(C)c1c[nH]c(=O)c2cc(NS(=O)(=O)c3cccc(Cl)c3Cl)ccc12] 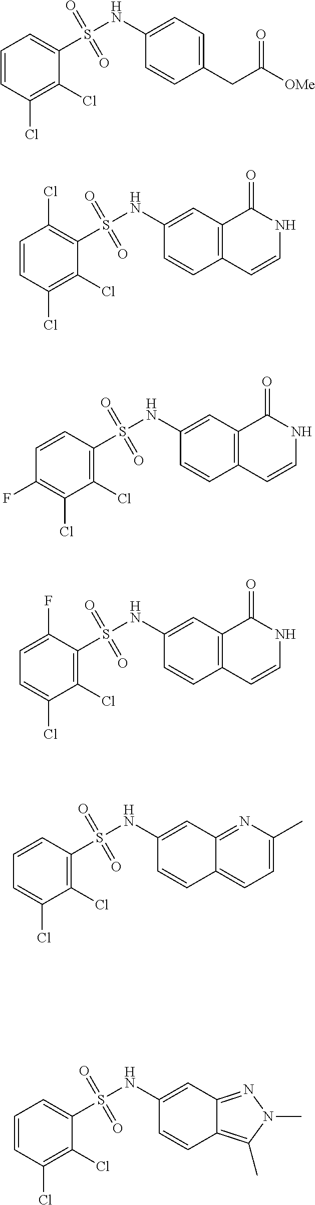 [SMILES: COC(=O)Cc1ccc(NS(=O)(=O)c2cccc(Cl)c2Cl)cc1 ; O=c1[nH]ccc2ccc(NS(=O)(=O)c3c(Cl)ccc(Cl)c3Cl)cc12 ; O=c1[nH]ccc2ccc(NS(=O)(=O)c3ccc(F)c(Cl)c3Cl)cc12 ; O=c1[nH]ccc2ccc(NS(=O)(=O)c3c(F)ccc(Cl)c3Cl)cc12 ; Cc1ccc2ccc(NS(=O)(=O)c3cccc(Cl)c3Cl)cc2n1 ; Cc1c2ccc(NS(=O)(=O)c3cccc(Cl)c3Cl)cc2nn1C] 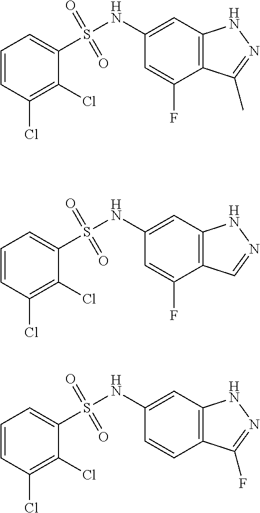 [SMILES: Cc1n[nH]c2cc(NS(=O)(=O)c3cccc(Cl)c3Cl)cc(F)c12 ; O=S(=O)(Nc1cc(F)c2cn[nH]c2c1)c1cccc(Cl)c1Cl ; O=S(=O)(Nc1ccc2c(F)n[nH]c2c1)c1cccc(Cl)c1Cl] 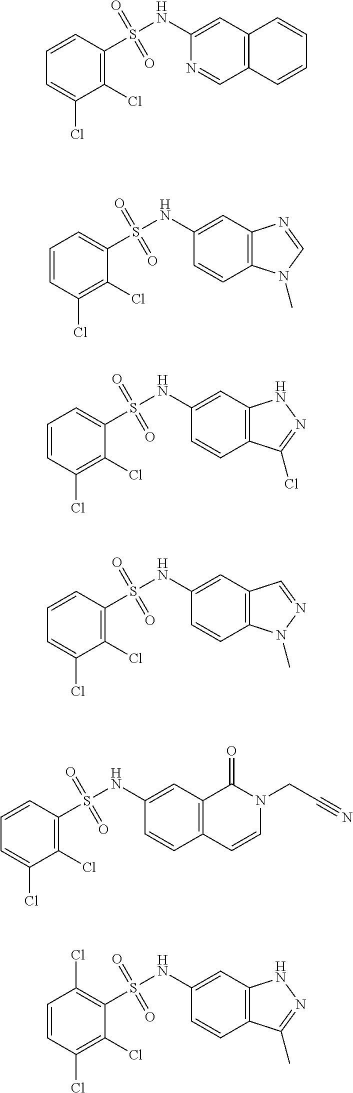 [SMILES: O=S(=O)(Nc1cc2ccccc2cn1)c1cccc(Cl)c1Cl ; Cn1cnc2cc(NS(=O)(=O)c3cccc(Cl)c3Cl)ccc21 ; O=S(=O)(Nc1ccc2c(Cl)n[nH]c2c1)c1cccc(Cl)c1Cl ; Cn1ncc2cc(NS(=O)(=O)c3cccc(Cl)c3Cl)ccc21 ; N#CCn1ccc2ccc(NS(=O)(=O)c3cccc(Cl)c3Cl)cc2c1=O ; Cc1n[nH]c2cc(NS(=O)(=O)c3c(Cl)ccc(Cl)c3Cl)ccc12] 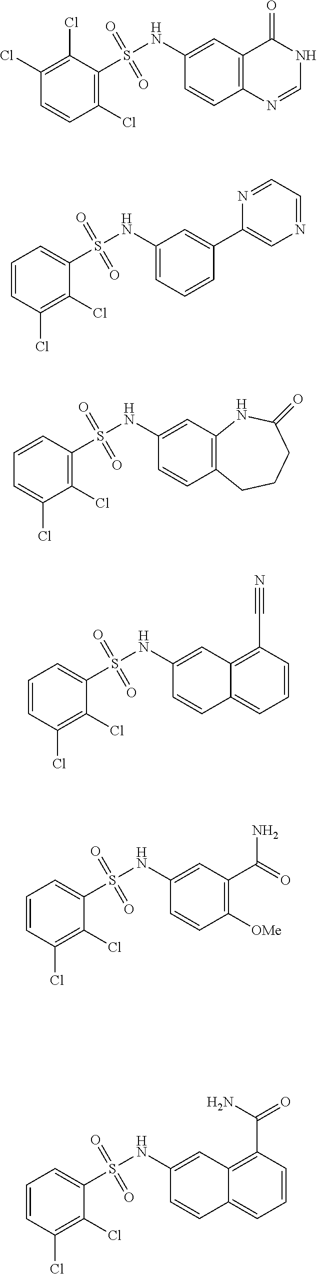 [SMILES: O=c1[nH]cnc2ccc(NS(=O)(=O)c3c(Cl)ccc(Cl)c3Cl)cc12 ; O=S(=O)(Nc1cccc(-c2cnccn2)c1)c1cccc(Cl)c1Cl ; O=C1CCCc2ccc(NS(=O)(=O)c3cccc(Cl)c3Cl)cc2N1 ; N#Cc1cccc2ccc(NS(=O)(=O)c3cccc(Cl)c3Cl)cc12 ; COc1ccc(NS(=O)(=O)c2cccc(Cl)c2Cl)cc1C(N)=O ; NC(=O)c1cccc2ccc(NS(=O)(=O)c3cccc(Cl)c3Cl)cc12] 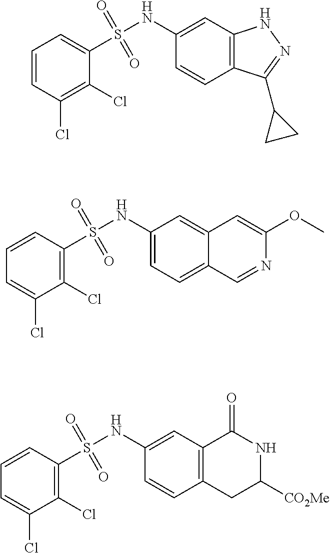 [SMILES: O=S(=O)(Nc1ccc2c(C3CC3)n[nH]c2c1)c1cccc(Cl)c1Cl ; COc1cc2cc(NS(=O)(=O)c3cccc(Cl)c3Cl)ccc2cn1 ; COC(=O)C1Cc2ccc(NS(=O)(=O)c3cccc(Cl)c3Cl)cc2C(=O)N1] 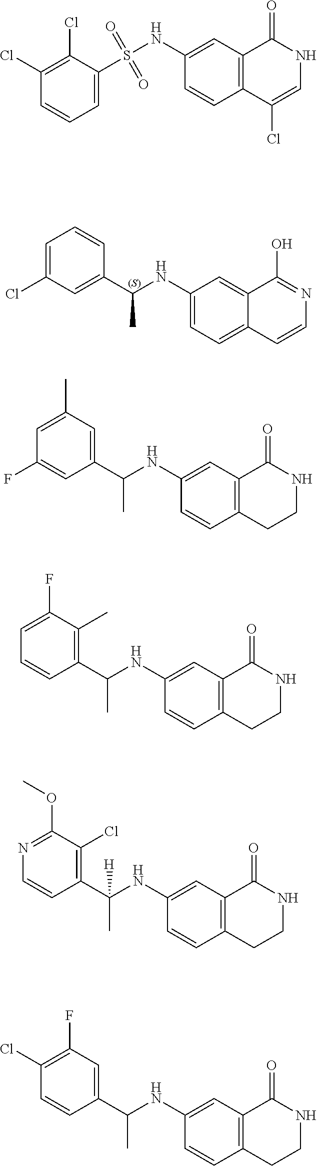 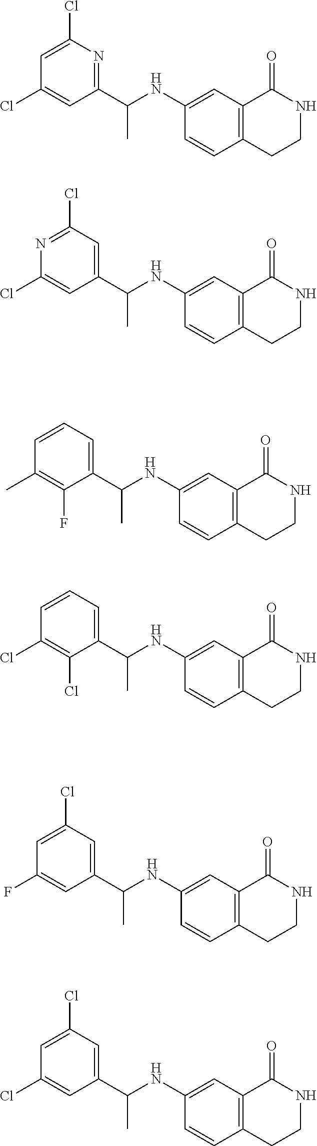 [SMILES: CC(Nc1ccc2c(c1)C(=O)NCC2)c1cc(Cl)cc(Cl)n1 ; CC(Nc1ccc2c(c1)C(=O)NCC2)c1cc(Cl)nc(Cl)c1 ; Cc1cccc(C(C)Nc2ccc3c(c2)C(=O)NCC3)c1F ; CC(Nc1ccc2c(c1)C(=O)NCC2)c1cccc(Cl)c1Cl ; CC(Nc1ccc2c(c1)C(=O)NCC2)c1cc(F)cc(Cl)c1 ; CC(Nc1ccc2c(c1)C(=O)NCC2)c1cc(Cl)cc(Cl)c1] 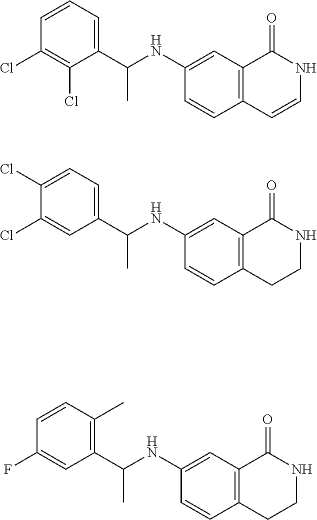 [SMILES: CC(Nc1ccc2cc[nH]c(=O)c2c1)c1cccc(Cl)c1Cl ; CC(Nc1ccc2c(c1)C(=O)NCC2)c1ccc(Cl)c(Cl)c1 ; Cc1ccc(F)cc1C(C)Nc1ccc2c(c1)C(=O)NCC2] 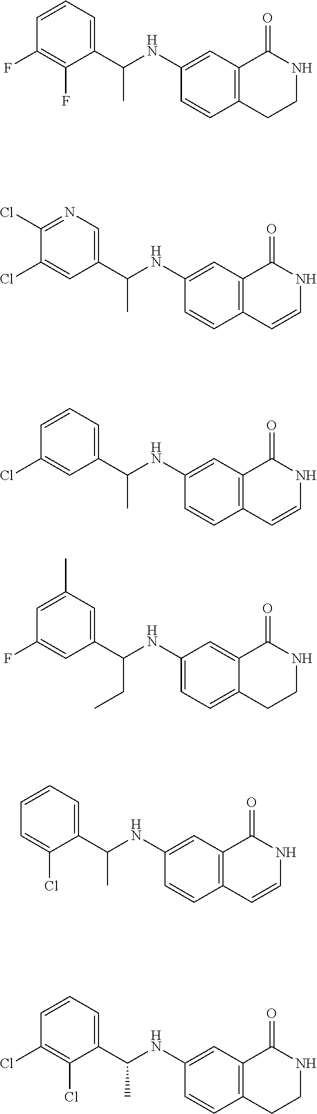 [SMILES: CC(Nc1ccc2c(c1)C(=O)NCC2)c1cccc(F)c1F ; CC(Nc1ccc2cc[nH]c(=O)c2c1)c1cnc(Cl)c(Cl)c1 ; CC(Nc1ccc2cc[nH]c(=O)c2c1)c1cccc(Cl)c1 ; CCC(Nc1ccc2c(c1)C(=O)NCC2)c1cc(C)cc(F)c1 ; CC(Nc1ccc2cc[nH]c(=O)c2c1)c1ccccc1Cl ; C[C@@H](Nc1ccc2c(c1)C(=O)NCC2)c1cccc(Cl)c1Cl] 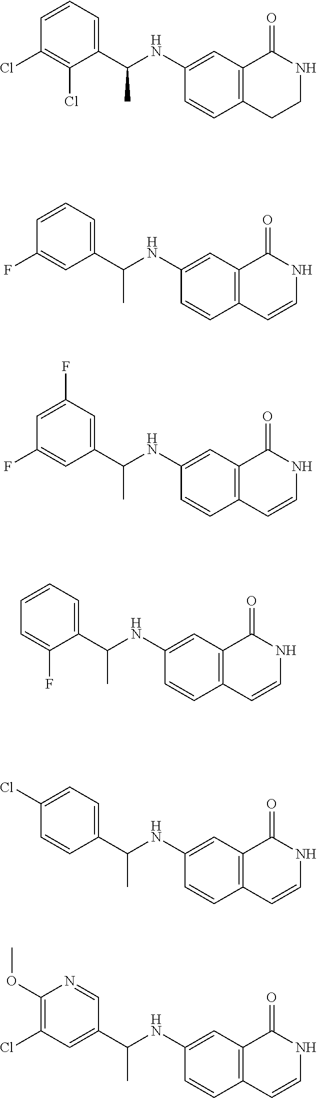 [SMILES: C[C@H](Nc1ccc2c(c1)C(=O)NCC2)c1cccc(Cl)c1Cl ; CC(Nc1ccc2cc[nH]c(=O)c2c1)c1cccc(F)c1 ; CC(Nc1ccc2cc[nH]c(=O)c2c1)c1cc(F)cc(F)c1 ; CC(Nc1ccc2cc[nH]c(=O)c2c1)c1ccccc1F ; CC(Nc1ccc2cc[nH]c(=O)c2c1)c1ccc(Cl)cc1 ; COc1ncc(C(C)Nc2ccc3cc[nH]c(=O)c3c2)cc1Cl] 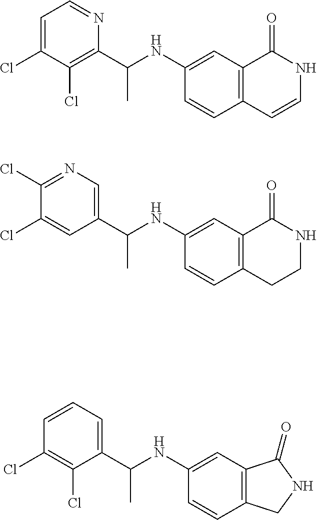 [SMILES: CC(Nc1ccc2cc[nH]c(=O)c2c1)c1nccc(Cl)c1Cl ; CC(Nc1ccc2c(c1)C(=O)NCC2)c1cnc(Cl)c(Cl)c1 ; CC(Nc1ccc2c(c1)C(=O)NC2)c1cccc(Cl)c1Cl] 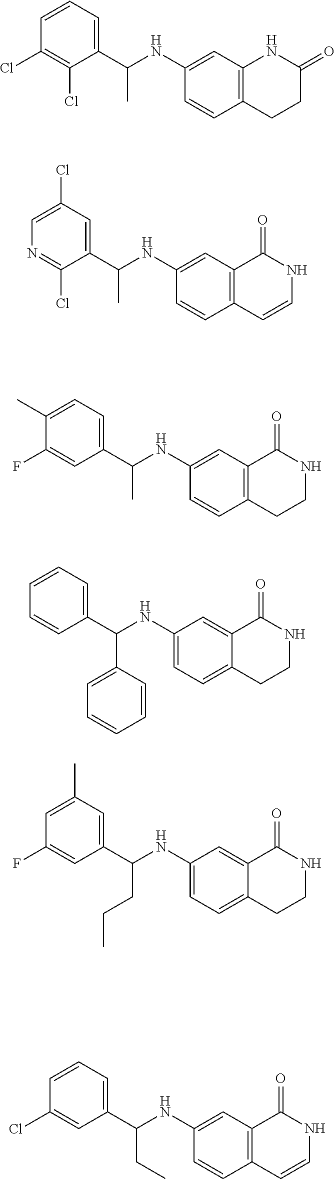 [SMILES: CC(Nc1ccc2c(c1)NC(=O)CC2)c1cccc(Cl)c1Cl ; CC(Nc1ccc2cc[nH]c(=O)c2c1)c1cc(Cl)cnc1Cl ; Cc1ccc(C(C)Nc2ccc3c(c2)C(=O)NCC3)cc1F ; O=C1NCCc2ccc(NC(c3ccccc3)c3ccccc3)cc21 ; CCCC(Nc1ccc2c(c1)C(=O)NCC2)c1cc(C)cc(F)c1 ; CCC(Nc1ccc2cc[nH]c(=O)c2c1)c1cccc(Cl)c1] 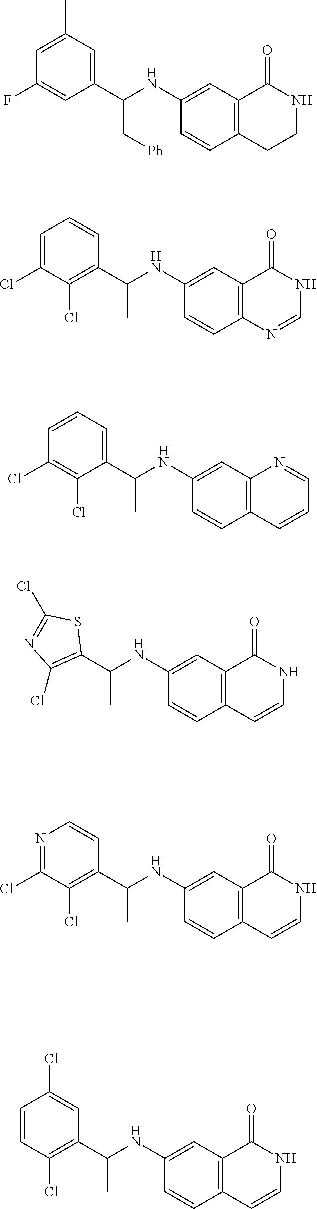 [SMILES: Cc1cc(F)cc(C(Cc2ccccc2)Nc2ccc3c(c2)C(=O)NCC3)c1 ; CC(Nc1ccc2nc[nH]c(=O)c2c1)c1cccc(Cl)c1Cl ; CC(Nc1ccc2cccnc2c1)c1cccc(Cl)c1Cl ; CC(Nc1ccc2cc[nH]c(=O)c2c1)c1sc(Cl)nc1Cl ; CC(Nc1ccc2cc[nH]c(=O)c2c1)c1ccnc(Cl)c1Cl ; CC(Nc1ccc2cc[nH]c(=O)c2c1)c1cc(Cl)ccc1Cl] 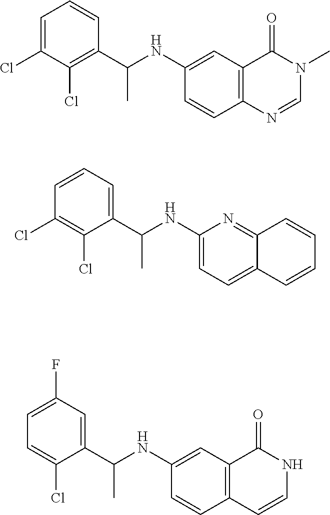 [SMILES: CC(Nc1ccc2ncn(C)c(=O)c2c1)c1cccc(Cl)c1Cl ; CC(Nc1ccc2ccccc2n1)c1cccc(Cl)c1Cl ; CC(Nc1ccc2cc[nH]c(=O)c2c1)c1cc(F)ccc1Cl] 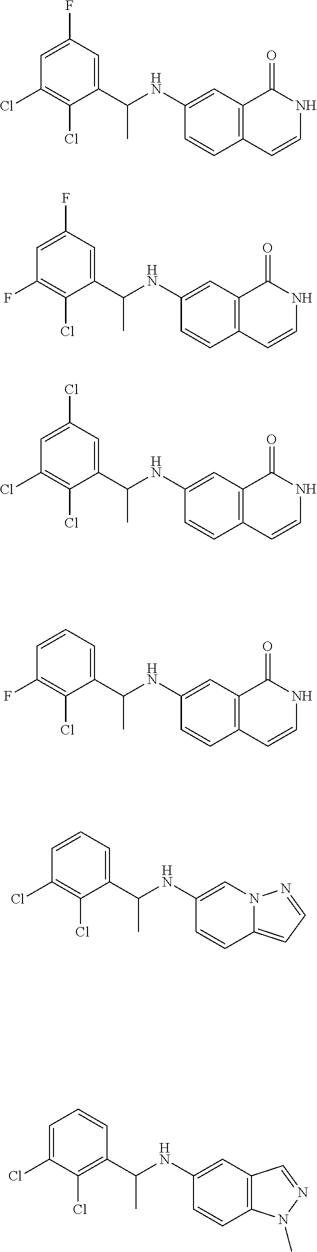 [SMILES: CC(Nc1ccc2cc[nH]c(=O)c2c1)c1cc(F)cc(Cl)c1Cl ; CC(Nc1ccc2cc[nH]c(=O)c2c1)c1cc(F)cc(F)c1Cl ; CC(Nc1ccc2cc[nH]c(=O)c2c1)c1cc(Cl)cc(Cl)c1Cl ; CC(Nc1ccc2cc[nH]c(=O)c2c1)c1cccc(F)c1Cl ; CC(Nc1ccc2ccnn2c1)c1cccc(Cl)c1Cl ; CC(Nc1ccc2c(cnn2C)c1)c1cccc(Cl)c1Cl] 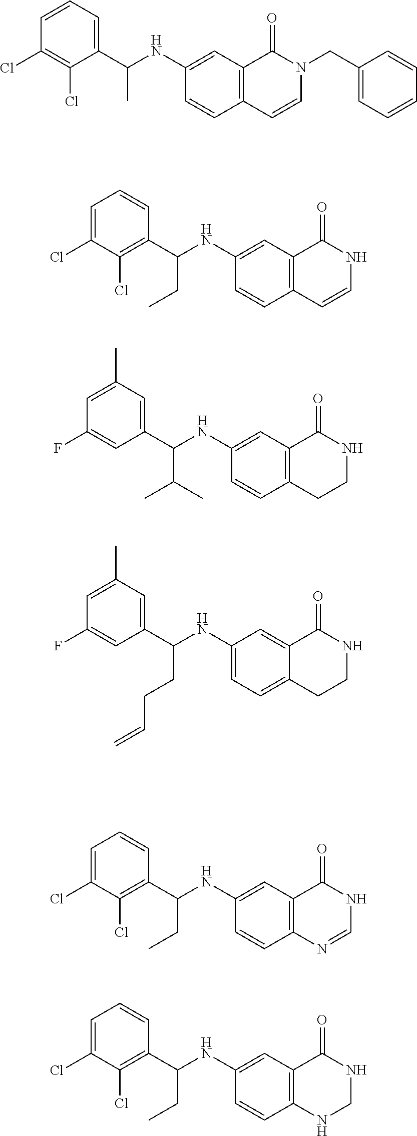 [SMILES: CC(Nc1ccc2ccn(Cc3ccccc3)c(=O)c2c1)c1cccc(Cl)c1Cl ; CCC(Nc1ccc2cc[nH]c(=O)c2c1)c1cccc(Cl)c1Cl ; Cc1cc(F)cc(C(Nc2ccc3c(c2)C(=O)NCC3)C(C)C)c1 ; C=CCCC(Nc1ccc2c(c1)C(=O)NCC2)c1cc(C)cc(F)c1 ; CCC(Nc1ccc2nc[nH]c(=O)c2c1)c1cccc(Cl)c1Cl ; CCC(Nc1ccc2c(c1)C(=O)NCN2)c1cccc(Cl)c1Cl] 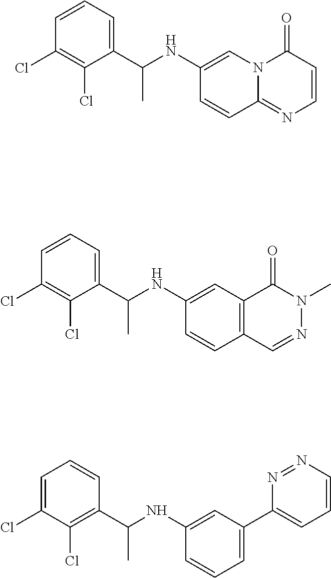 [SMILES: CC(Nc1ccc2nccc(=O)n2c1)c1cccc(Cl)c1Cl ; CC(Nc1ccc2cnn(C)c(=O)c2c1)c1cccc(Cl)c1Cl ; CC(Nc1cccc(-c2cccnn2)c1)c1cccc(Cl)c1Cl] 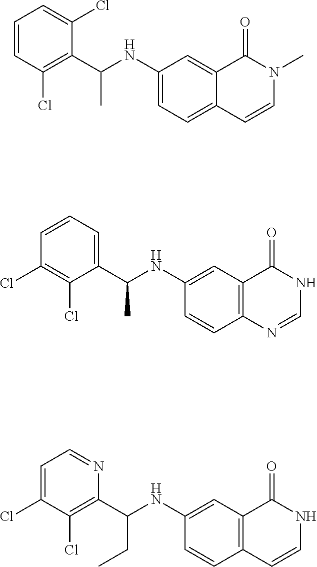 [SMILES: CC(Nc1ccc2ccn(C)c(=O)c2c1)c1c(Cl)cccc1Cl ; C[C@H](Nc1ccc2nc[nH]c(=O)c2c1)c1cccc(Cl)c1Cl ; CCC(Nc1ccc2cc[nH]c(=O)c2c1)c1nccc(Cl)c1Cl] 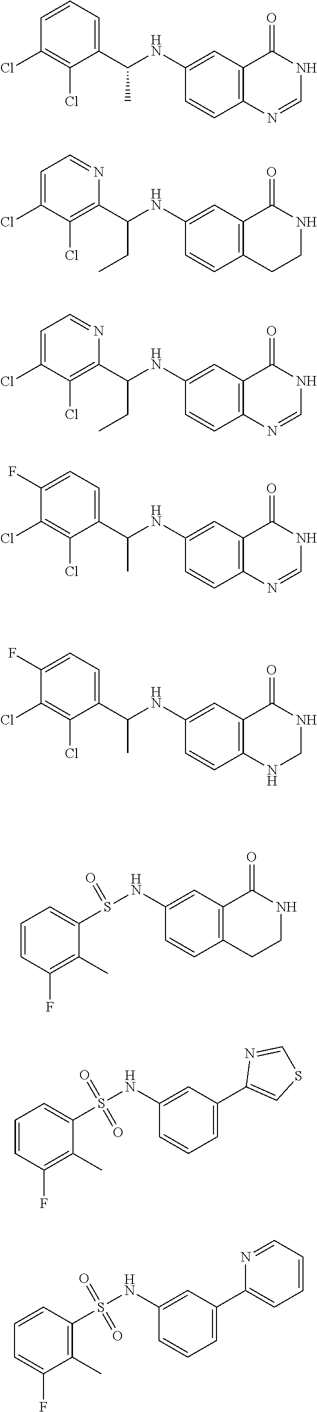 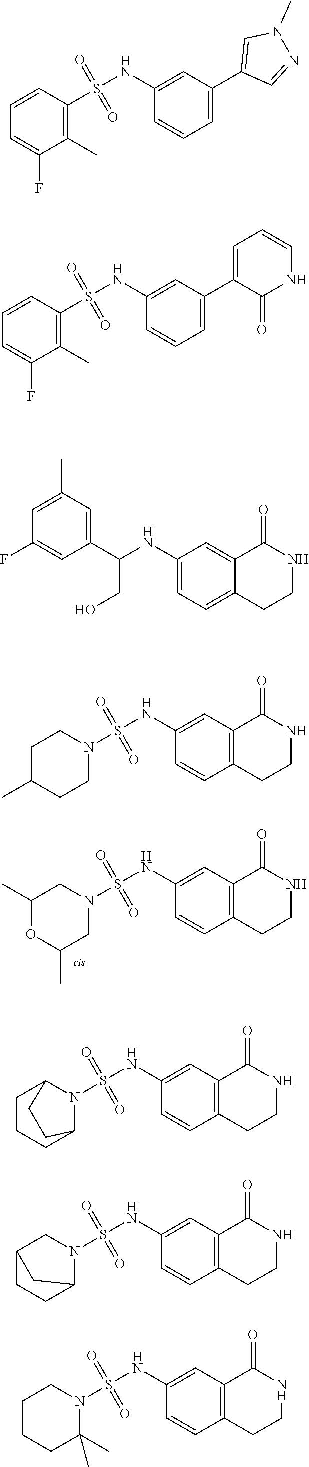 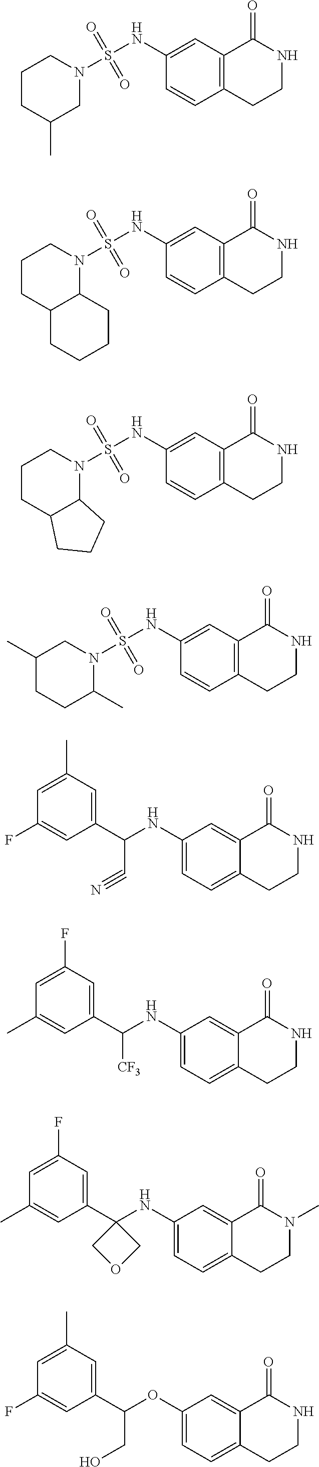 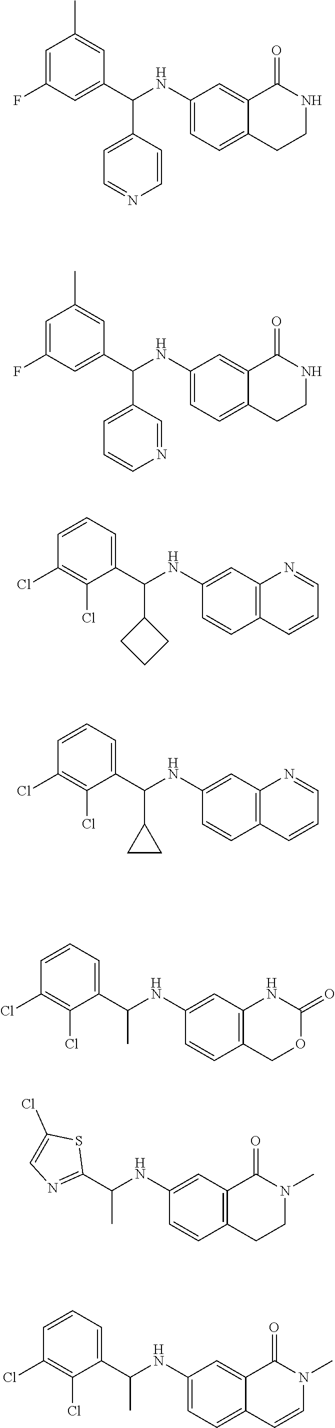 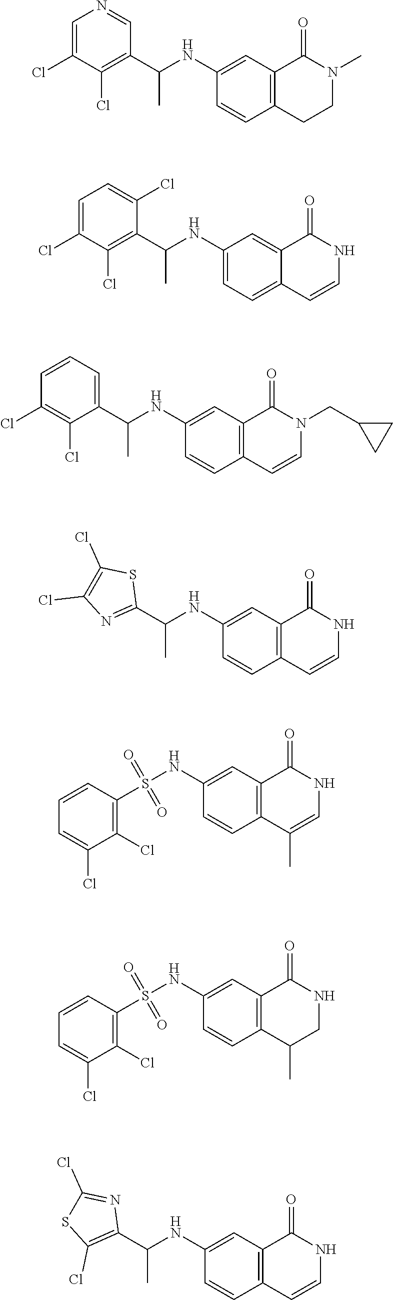 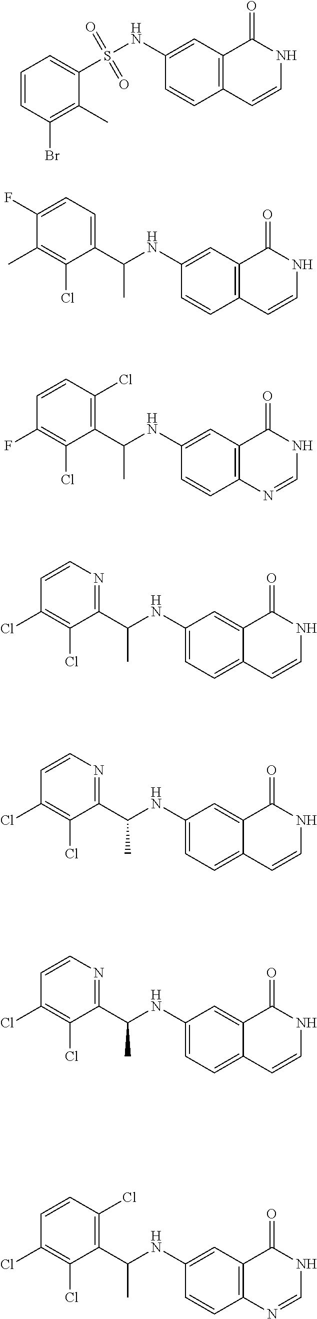 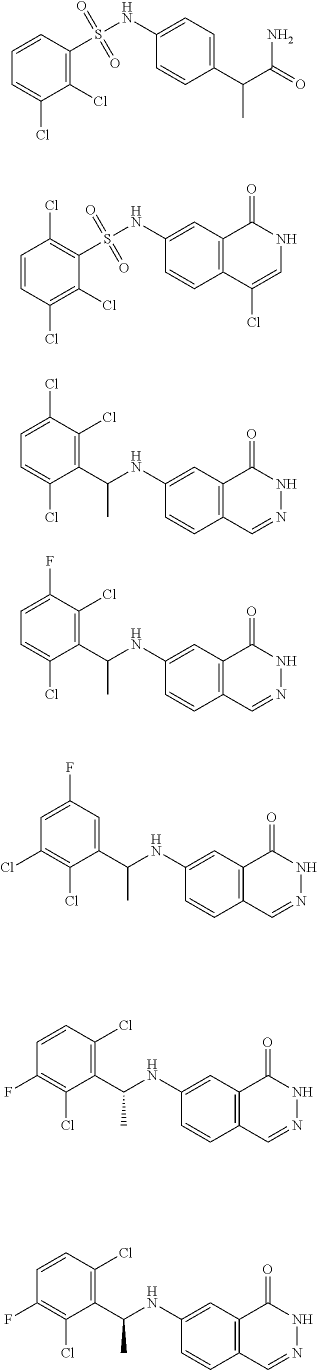 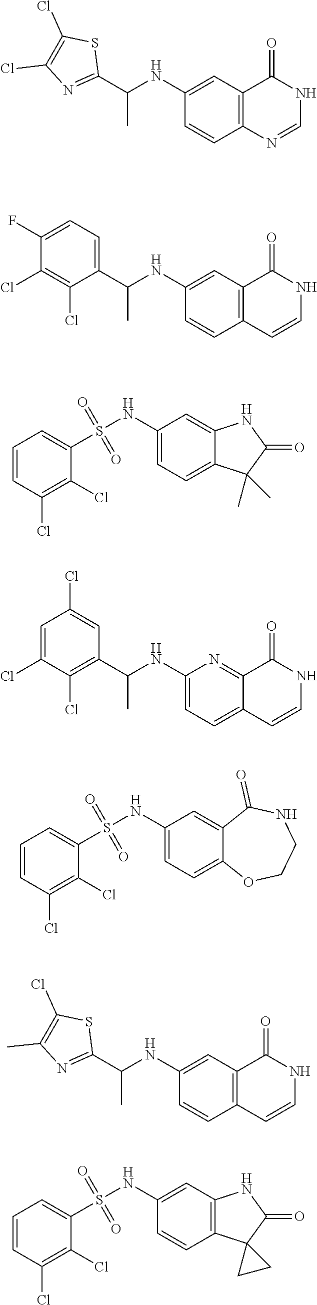 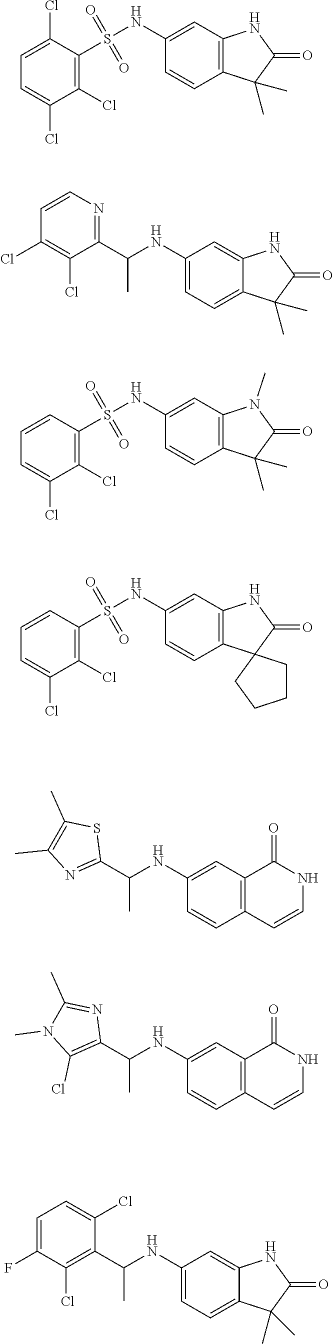 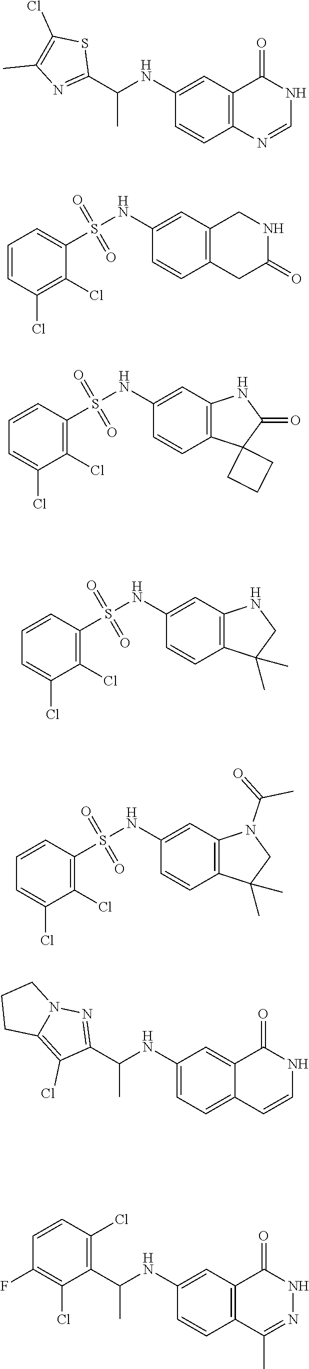 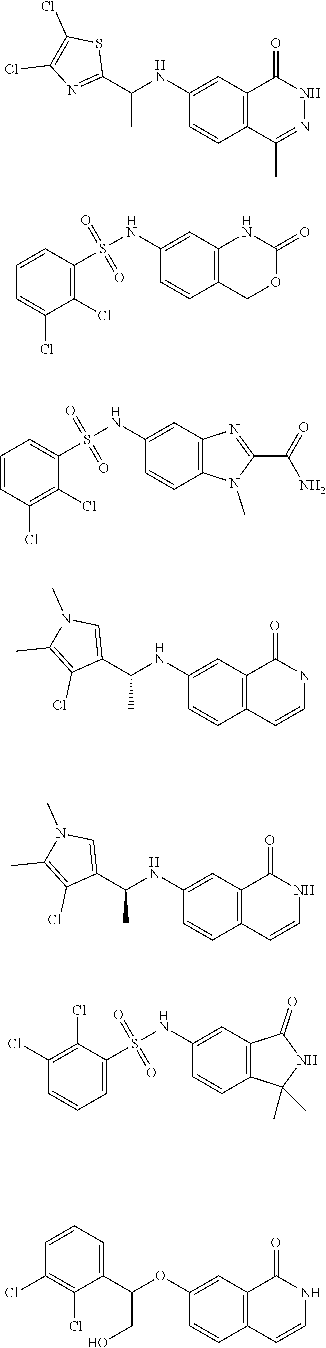 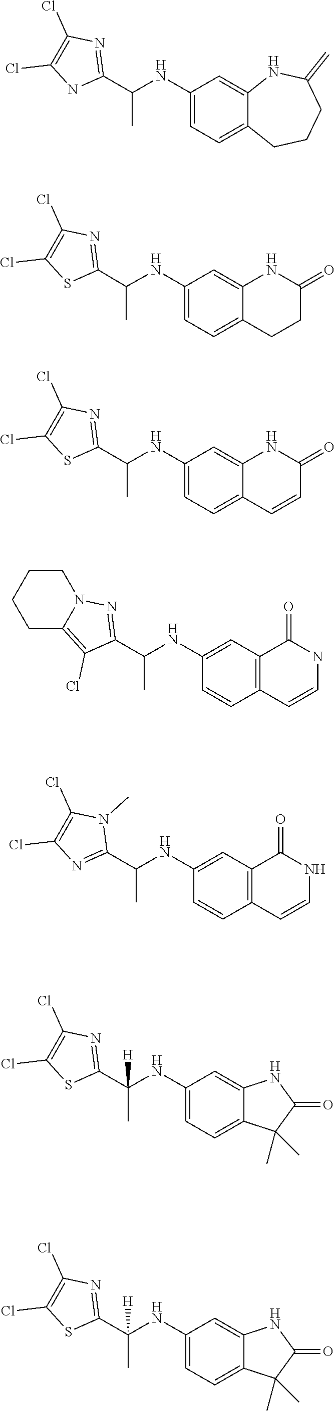 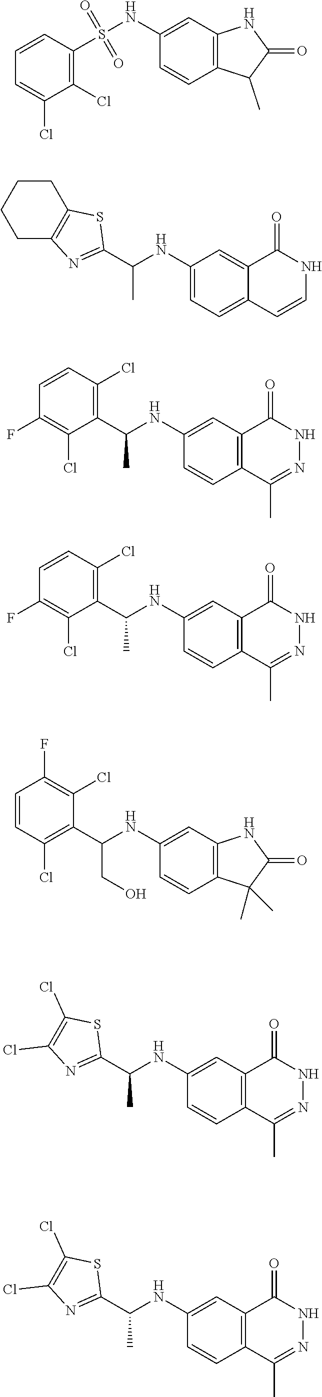 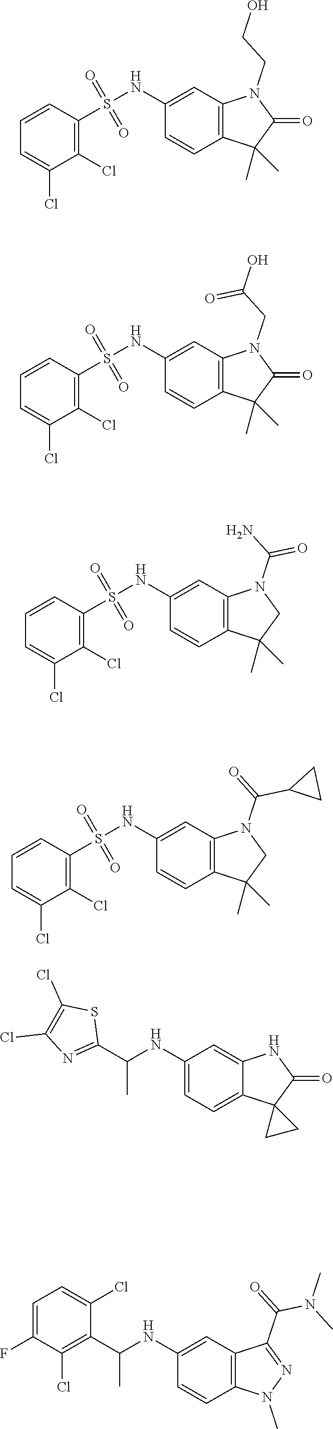 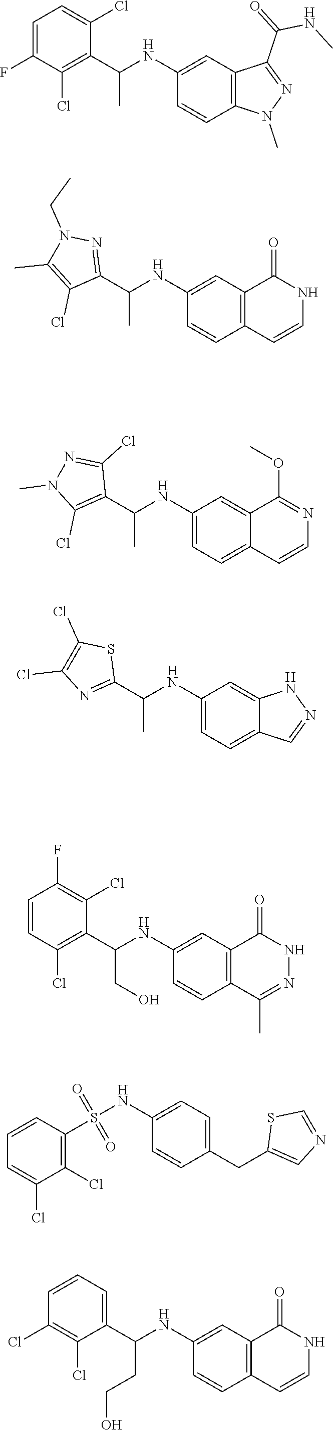 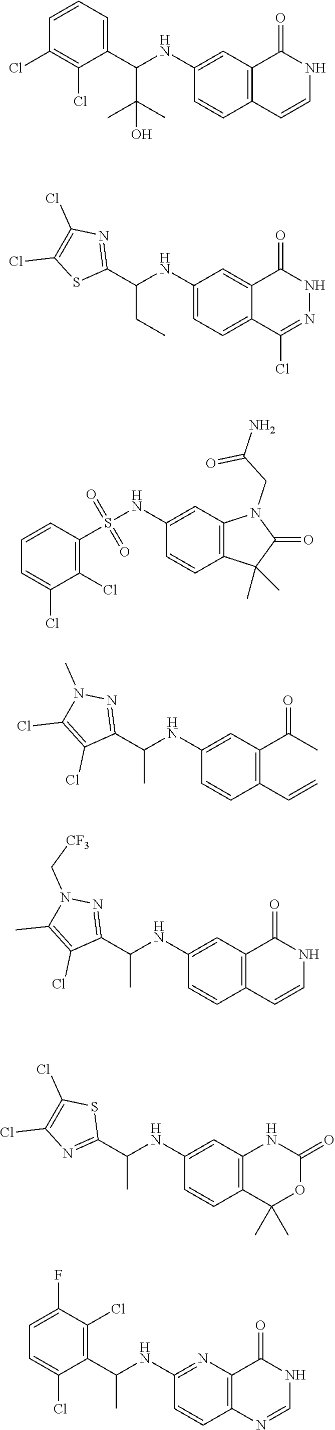 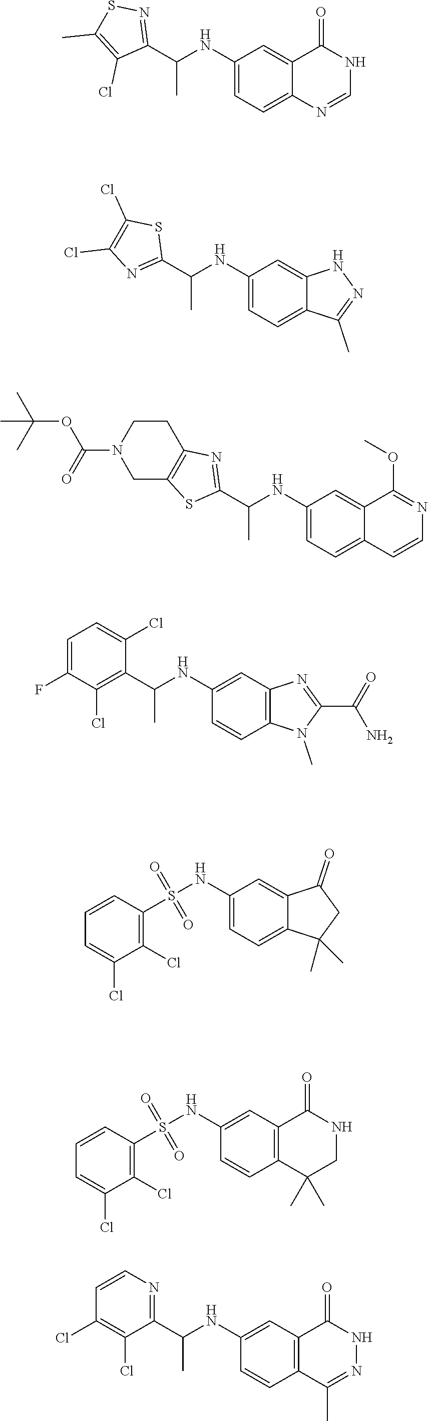 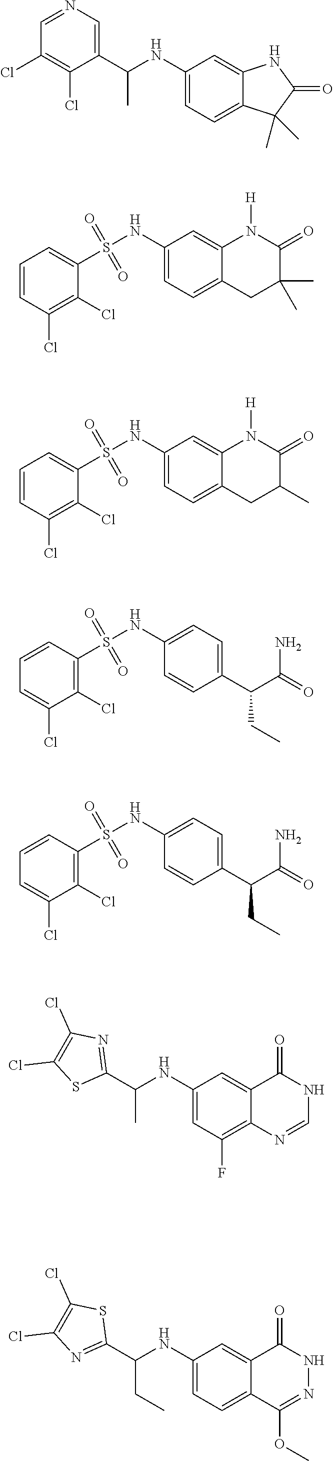 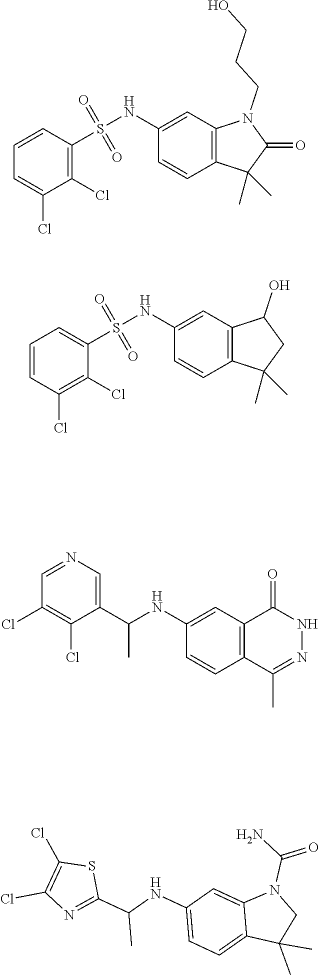 [SMILES: CC1(C)C(=O)N(CCCO)c2cc(NS(=O)(=O)c3cccc(Cl)c3Cl)ccc21 ; CC1(C)CC(O)c2cc(NS(=O)(=O)c3cccc(Cl)c3Cl)ccc21 ; Cc1n[nH]c(=O)c2cc(NC(C)c3cncc(Cl)c3Cl)ccc12 ; CC(Nc1ccc2c(c1)N(C(N)=O)CC2(C)C)c1nc(Cl)c(Cl)s1] 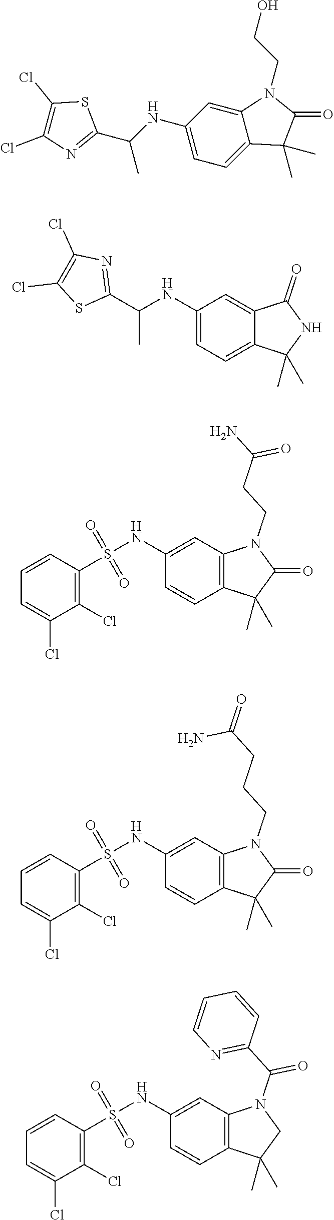 [SMILES: CC(Nc1ccc2c(c1)N(CCO)C(=O)C2(C)C)c1nc(Cl)c(Cl)s1 ; CC(Nc1ccc2c(c1)C(=O)NC2(C)C)c1nc(Cl)c(Cl)s1 ; CC1(C)C(=O)N(CCC(N)=O)c2cc(NS(=O)(=O)c3cccc(Cl)c3Cl)ccc21 ; CC1(C)C(=O)N(CCCC(N)=O)c2cc(NS(=O)(=O)c3cccc(Cl)c3Cl)ccc21 ; CC1(C)CN(C(=O)c2ccccn2)c2cc(NS(=O)(=O)c3cccc(Cl)c3Cl)ccc21] 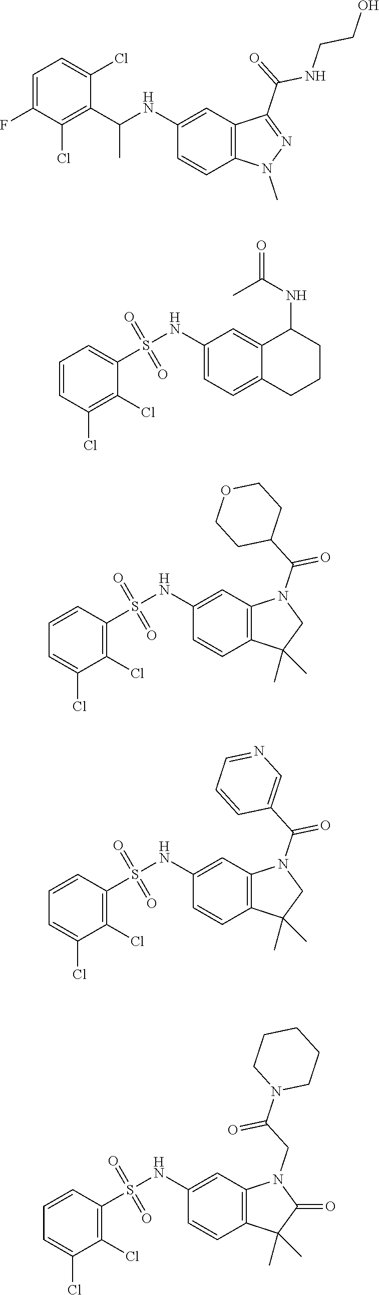 [SMILES: CC(Nc1ccc2c(c1)c(C(=O)NCCO)nn2C)c1c(Cl)ccc(F)c1Cl ; CC(=O)NC1CCCc2ccc(NS(=O)(=O)c3cccc(Cl)c3Cl)cc21 ; CC1(C)CN(C(=O)C2CCOCC2)c2cc(NS(=O)(=O)c3cccc(Cl)c3Cl)ccc21 ; CC1(C)CN(C(=O)c2cccnc2)c2cc(NS(=O)(=O)c3cccc(Cl)c3Cl)ccc21 ; CC1(C)C(=O)N(CC(=O)N2CCCCC2)c2cc(NS(=O)(=O)c3cccc(Cl)c3Cl)ccc21] 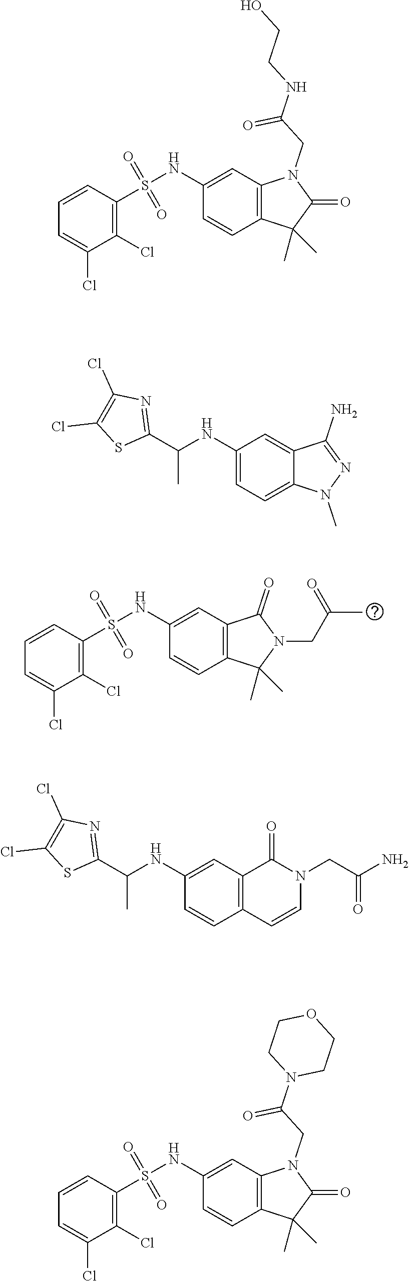 [SMILES: CC1(C)C(=O)N(CC(=O)NCCO)c2cc(NS(=O)(=O)c3cccc(Cl)c3Cl)ccc21 ; CC(Nc1ccc2c(c1)c(N)nn2C)c1nc(Cl)c(Cl)s1 ; CC(=O)CN1C(=O)c2cc(NS(=O)(=O)c3cccc(Cl)c3Cl)ccc2C1(C)C ; CC(Nc1ccc2ccn(CC(N)=O)c(=O)c2c1)c1nc(Cl)c(Cl)s1 ; CC1(C)C(=O)N(CC(=O)N2CCOCC2)c2cc(NS(=O)(=O)c3cccc(Cl)c3Cl)ccc21] 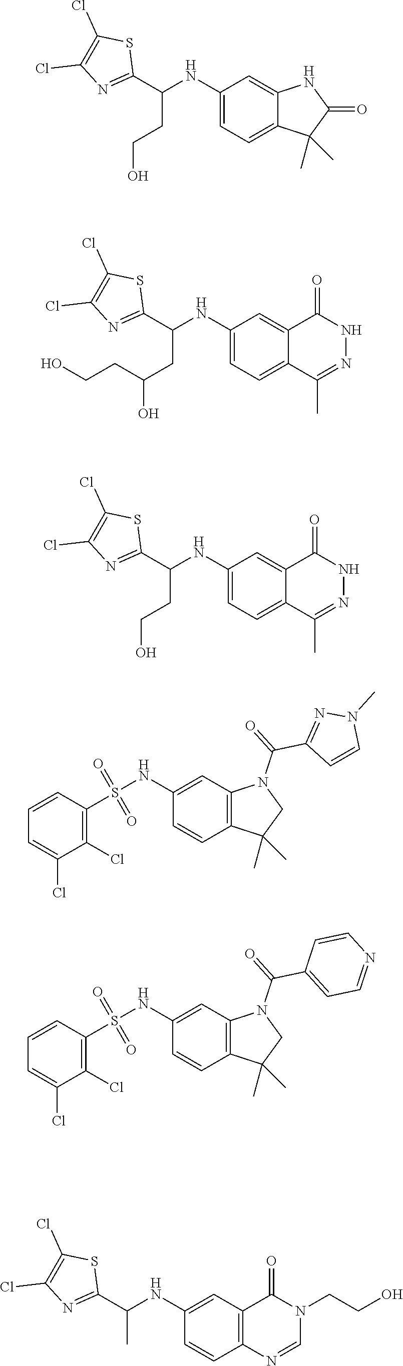 [SMILES: CC1(C)C(=O)Nc2cc(NC(CCO)c3nc(Cl)c(Cl)s3)ccc21 ; Cc1n[nH]c(=O)c2cc(NC(CC(O)CCO)c3nc(Cl)c(Cl)s3)ccc12 ; Cc1n[nH]c(=O)c2cc(NC(CCO)c3nc(Cl)c(Cl)s3)ccc12 ; Cn1ccc(C(=O)N2CC(C)(C)c3ccc(NS(=O)(=O)c4cccc(Cl)c4Cl)cc32)n1 ; CC1(C)CN(C(=O)c2ccncc2)c2cc(NS(=O)(=O)c3cccc(Cl)c3Cl)ccc21 ; CC(Nc1ccc2ncn(CCO)c(=O)c2c1)c1nc(Cl)c(Cl)s1] 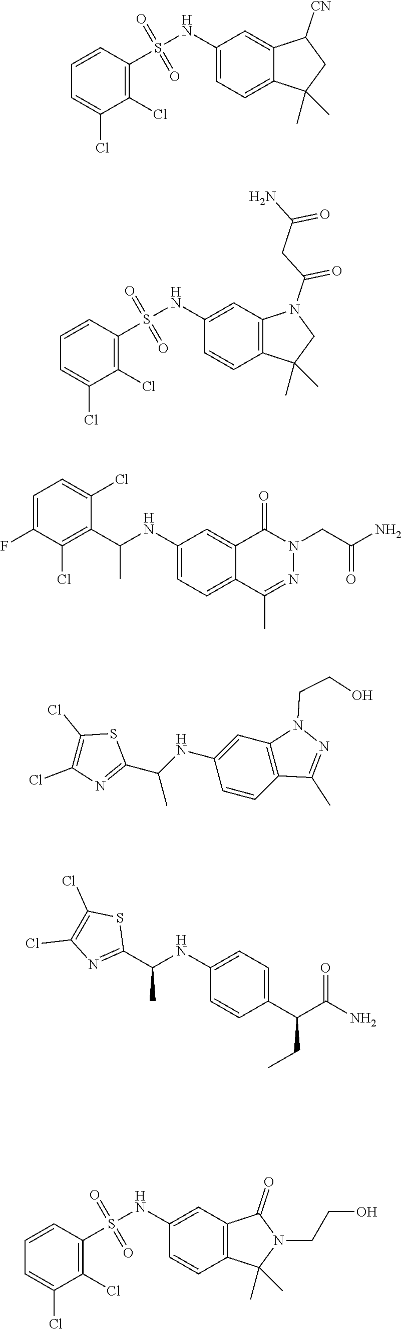 [SMILES: CC1(C)CC(C#N)c2cc(NS(=O)(=O)c3cccc(Cl)c3Cl)ccc21 ; CC1(C)CN(C(=O)CC(N)=O)c2cc(NS(=O)(=O)c3cccc(Cl)c3Cl)ccc21 ; Cc1nn(CC(N)=O)c(=O)c2cc(NC(C)c3c(Cl)ccc(F)c3Cl)ccc12 ; Cc1nn(CCO)c2cc(NC(C)c3nc(Cl)c(Cl)s3)ccc12 ; CC[C@H](C(N)=O)c1ccc(N[C@@H](C)c2nc(Cl)c(Cl)s2)cc1 ; CC1(C)c2ccc(NS(=O)(=O)c3cccc(Cl)c3Cl)cc2C(=O)N1CCO] 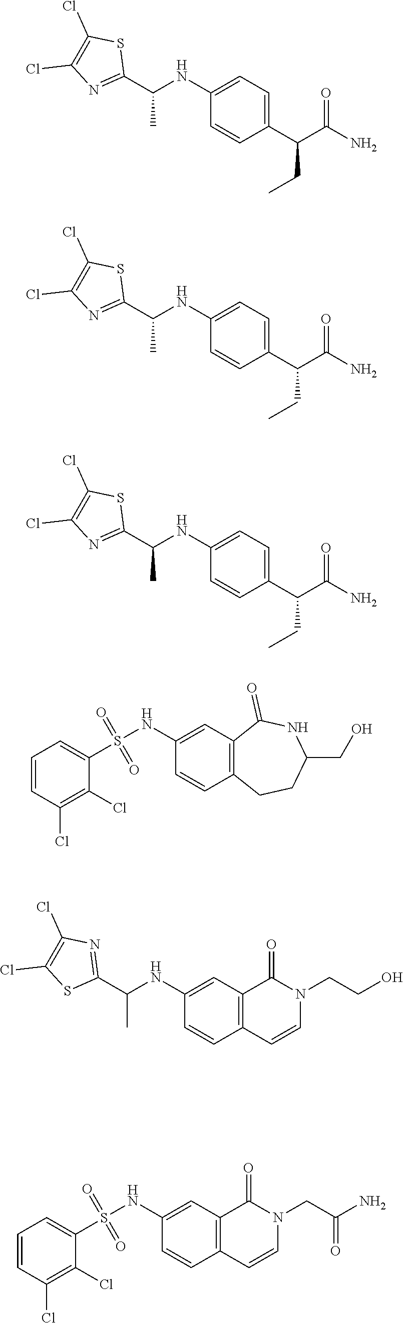 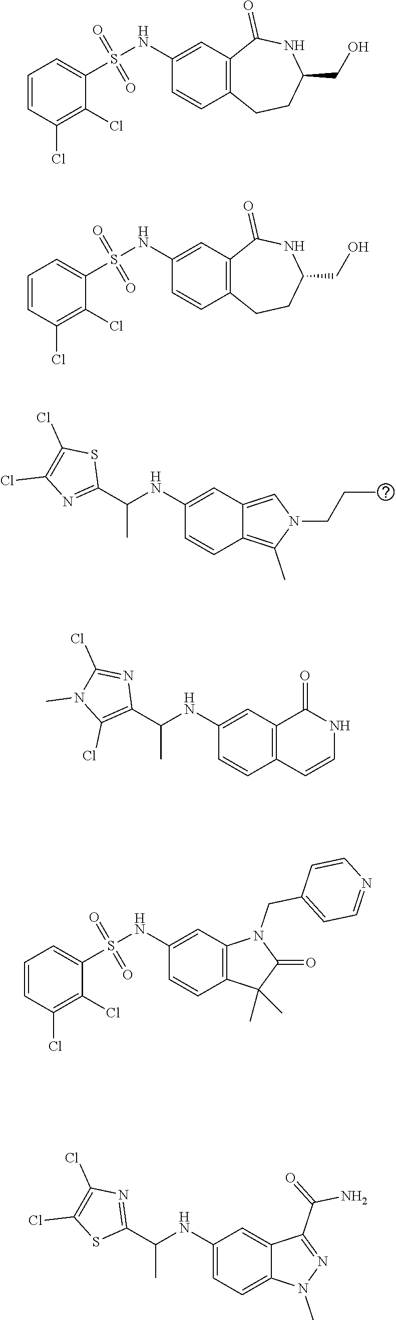 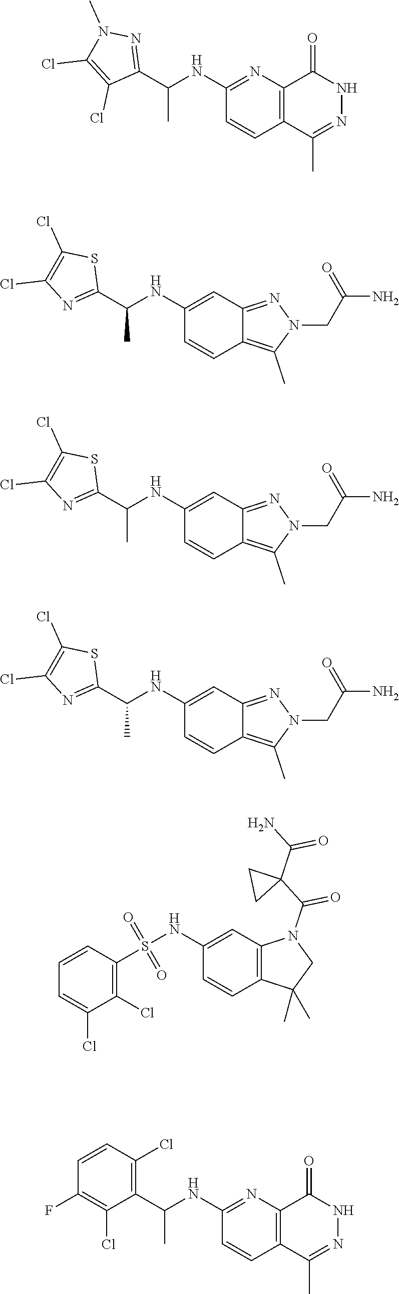 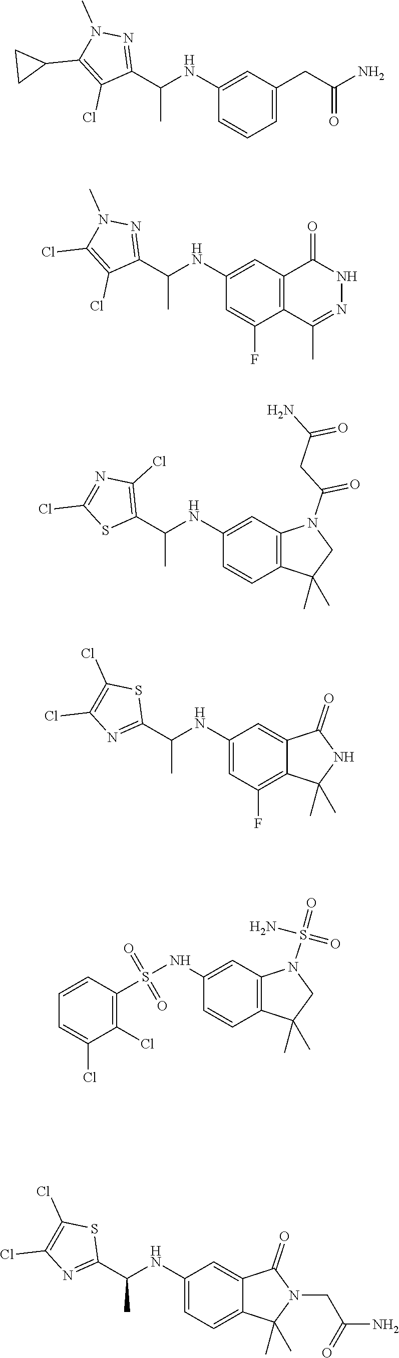 [SMILES: CC(Nc1cccc(CC(N)=O)c1)c1nn(C)c(C2CC2)c1Cl ; Cc1n[nH]c(=O)c2cc(NC(C)c3nn(C)c(Cl)c3Cl)cc(F)c12 ; CC(Nc1ccc2c(c1)N(C(=O)CC(N)=O)CC2(C)C)c1sc(Cl)nc1Cl ; CC(Nc1cc(F)c2c(c1)C(=O)NC2(C)C)c1nc(Cl)c(Cl)s1 ; CC1(C)CN(S(N)(=O)=O)c2cc(NS(=O)(=O)c3cccc(Cl)c3Cl)ccc21 ; C[C@H](Nc1ccc2c(c1)C(=O)N(CC(N)=O)C2(C)C)c1nc(Cl)c(Cl)s1] 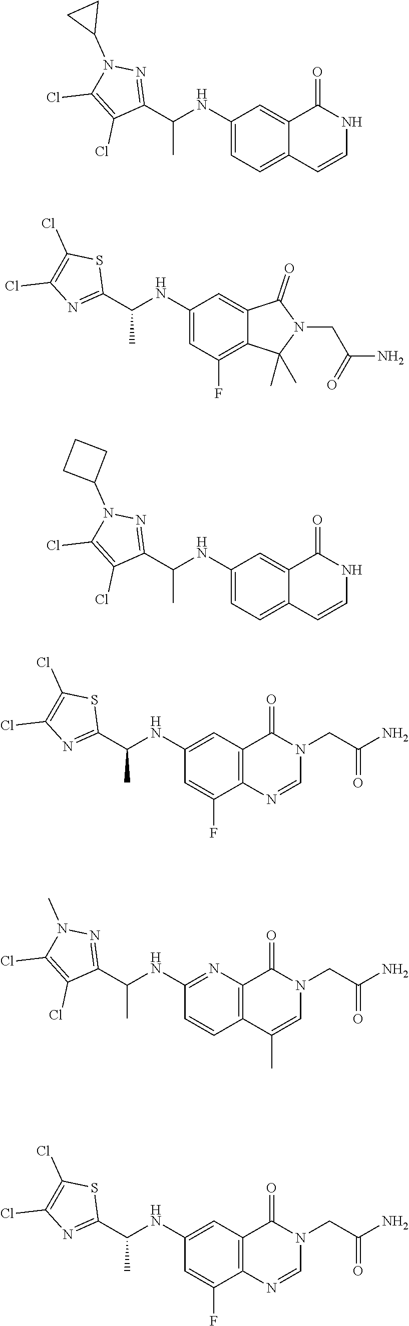 [SMILES: CC(Nc1ccc2cc[nH]c(=O)c2c1)c1nn(C2CC2)c(Cl)c1Cl ; C[C@@H](Nc1cc(F)c2c(c1)C(=O)N(CC(N)=O)C2(C)C)c1nc(Cl)c(Cl)s1 ; CC(Nc1ccc2cc[nH]c(=O)c2c1)c1nn(C2CCC2)c(Cl)c1Cl ; C[C@H](Nc1cc(F)c2ncn(CC(N)=O)c(=O)c2c1)c1nc(Cl)c(Cl)s1 ; Cc1cn(CC(N)=O)c(=O)c2nc(NC(C)c3nn(C)c(Cl)c3Cl)ccc12 ; C[C@@H](Nc1cc(F)c2ncn(CC(N)=O)c(=O)c2c1)c1nc(Cl)c(Cl)s1] 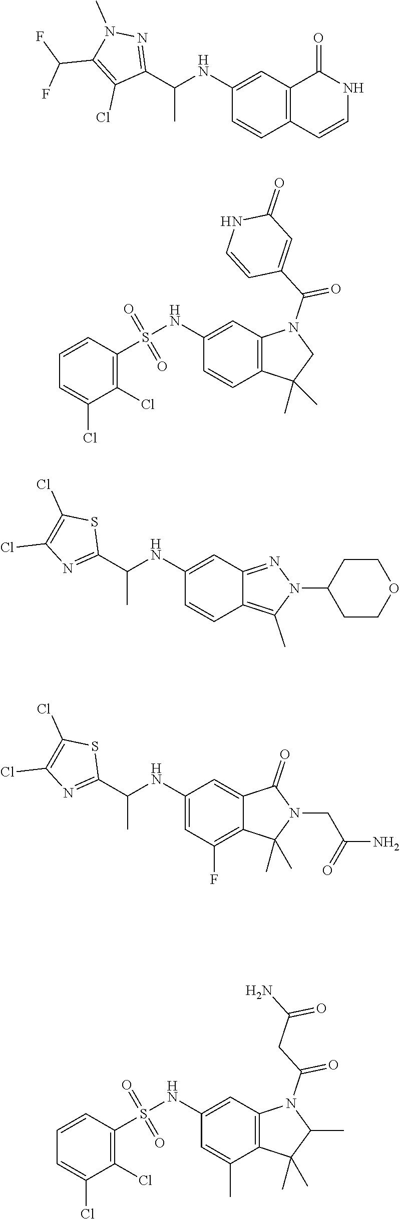 [SMILES: CC(Nc1ccc2cc[nH]c(=O)c2c1)c1nn(C)c(C(F)F)c1Cl ; CC1(C)CN(C(=O)c2cc[nH]c(=O)c2)c2cc(NS(=O)(=O)c3cccc(Cl)c3Cl)ccc21 ; Cc1c2ccc(NC(C)c3nc(Cl)c(Cl)s3)cc2nn1C1CCOCC1 ; CC(Nc1cc(F)c2c(c1)C(=O)N(CC(N)=O)C2(C)C)c1nc(Cl)c(Cl)s1 ; Cc1cc(NS(=O)(=O)c2cccc(Cl)c2Cl)cc2c1C(C)(C)C(C)N2C(=O)CC(N)=O] 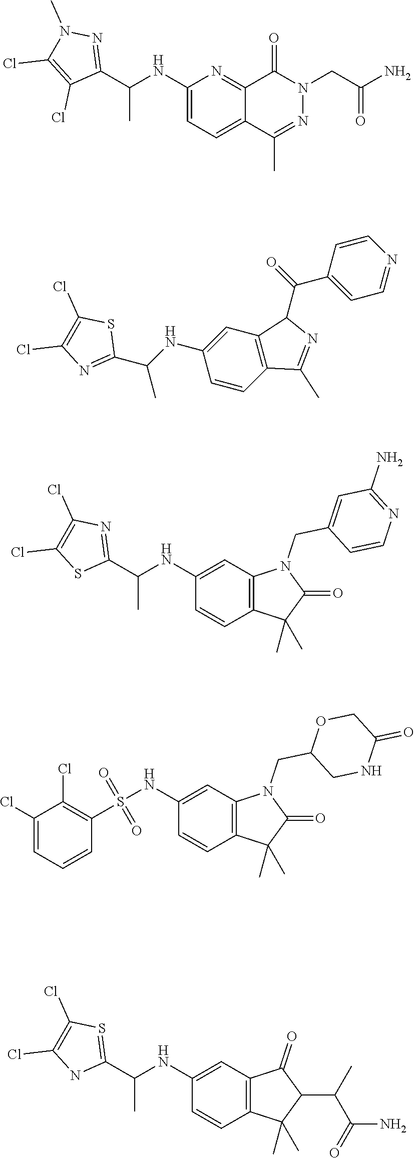 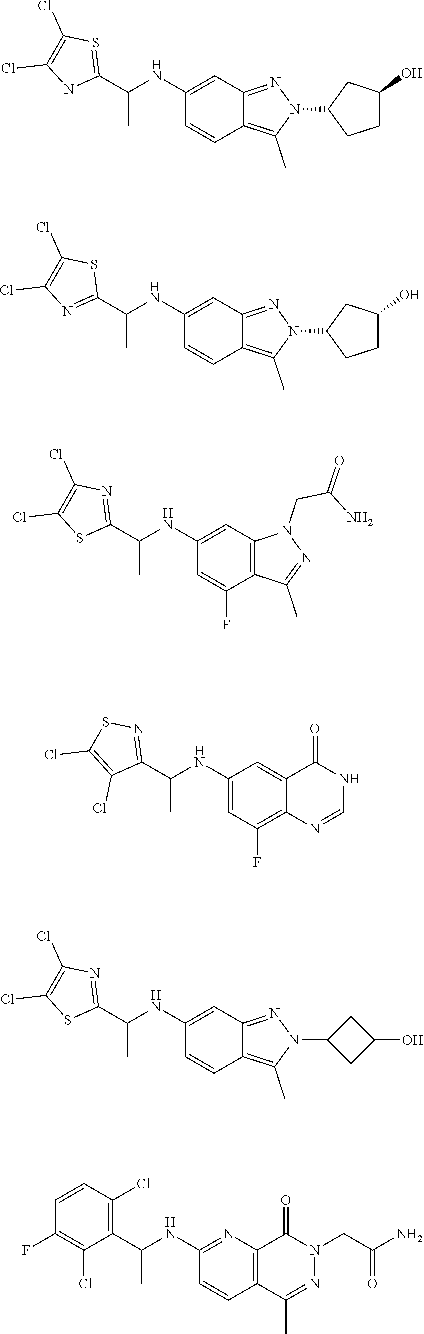 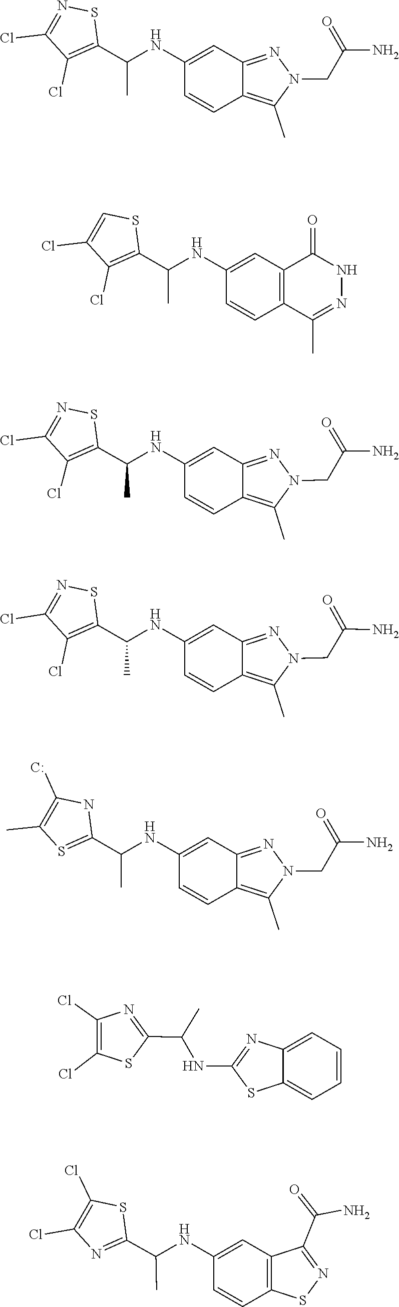 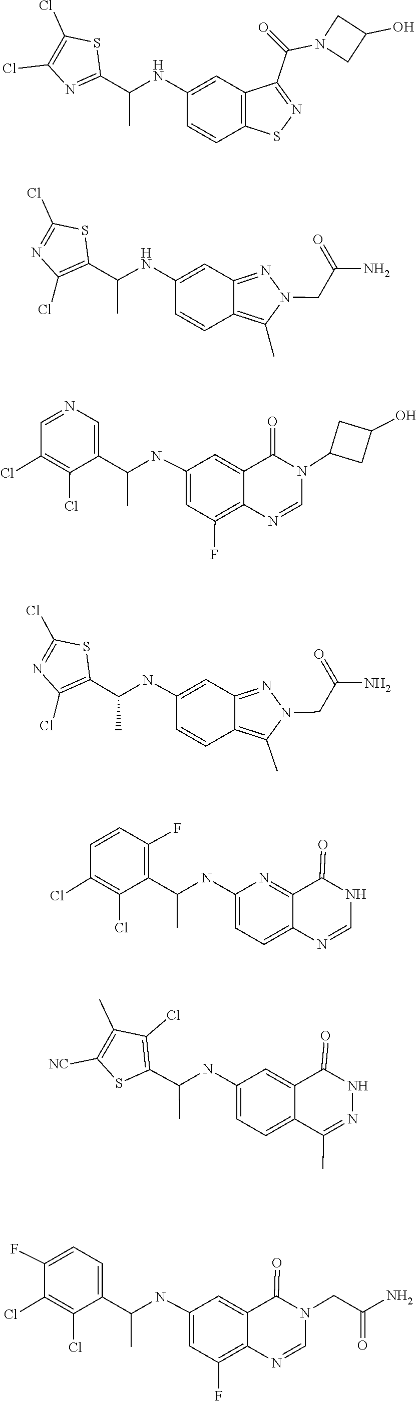 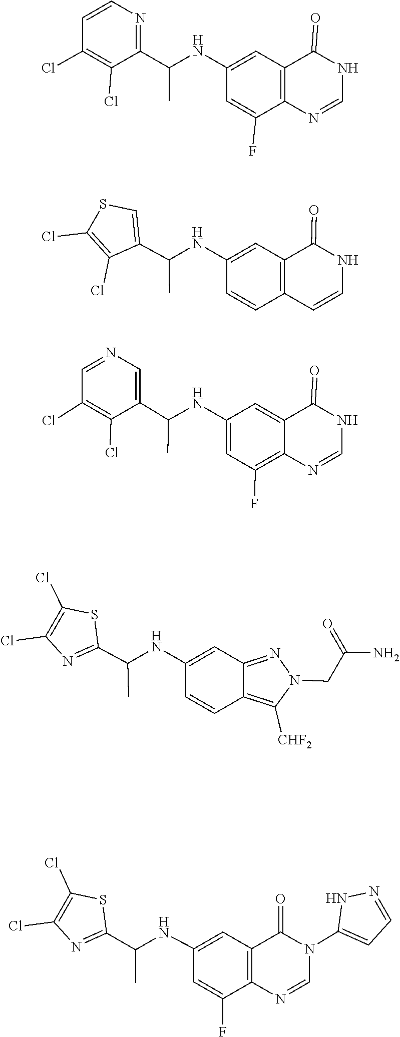 [SMILES: CC(Nc1cc(F)c2nc[nH]c(=O)c2c1)c1nccc(Cl)c1Cl ; CC(Nc1ccc2cc[nH]c(=O)c2c1)c1csc(Cl)c1Cl ; CC(Nc1cc(F)c2nc[nH]c(=O)c2c1)c1cncc(Cl)c1Cl ; CC(Nc1ccc2c(C(F)F)n(CC(N)=O)nc2c1)c1nc(Cl)c(Cl)s1 ; CC(Nc1cc(F)c2ncn(-c3ccn[nH]3)c(=O)c2c1)c1nc(Cl)c(Cl)s1] 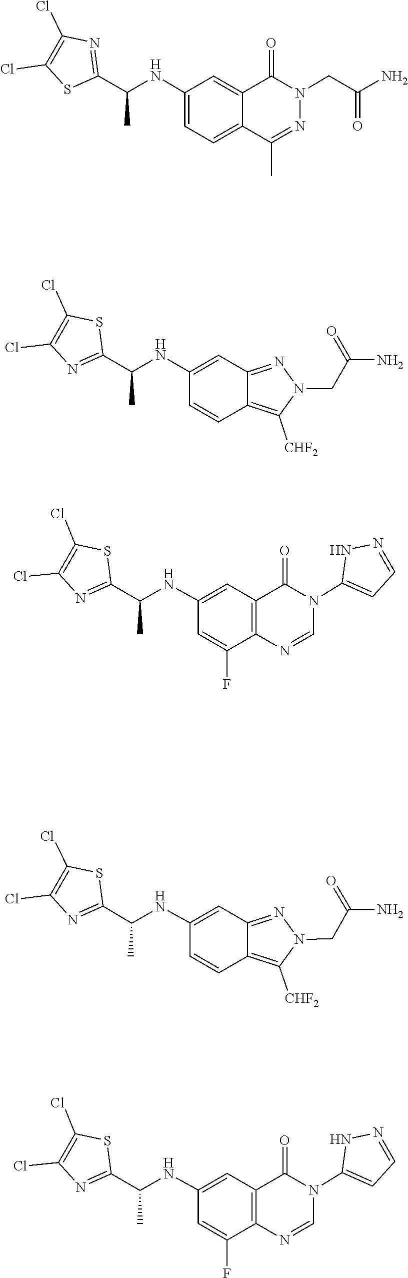 [SMILES: Cc1nn(CC(N)=O)c(=O)c2cc(N[C@@H](C)c3nc(Cl)c(Cl)s3)ccc12 ; C[C@H](Nc1ccc2c(C(F)F)n(CC(N)=O)nc2c1)c1nc(Cl)c(Cl)s1 ; C[C@H](Nc1cc(F)c2ncn(-c3ccn[nH]3)c(=O)c2c1)c1nc(Cl)c(Cl)s1 ; C[C@@H](Nc1ccc2c(C(F)F)n(CC(N)=O)nc2c1)c1nc(Cl)c(Cl)s1 ; C[C@@H](Nc1cc(F)c2ncn(-c3ccn[nH]3)c(=O)c2c1)c1nc(Cl)c(Cl)s1] 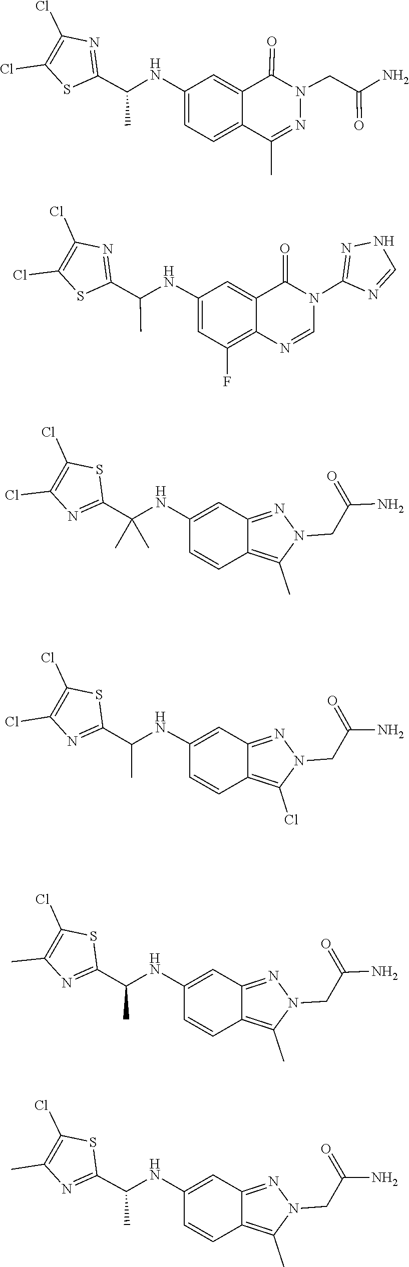 [SMILES: Cc1nn(CC(N)=O)c(=O)c2cc(N[C@H](C)c3nc(Cl)c(Cl)s3)ccc12 ; CC(Nc1cc(F)c2ncn(-c3nc[nH]n3)c(=O)c2c1)c1nc(Cl)c(Cl)s1 ; Cc1c2ccc(NC(C)(C)c3nc(Cl)c(Cl)s3)cc2nn1CC(N)=O ; CC(Nc1ccc2c(Cl)n(CC(N)=O)nc2c1)c1nc(Cl)c(Cl)s1 ; Cc1nc([C@H](C)Nc2ccc3c(C)n(CC(N)=O)nc3c2)sc1Cl ; Cc1nc([C@@H](C)Nc2ccc3c(C)n(CC(N)=O)nc3c2)sc1Cl] 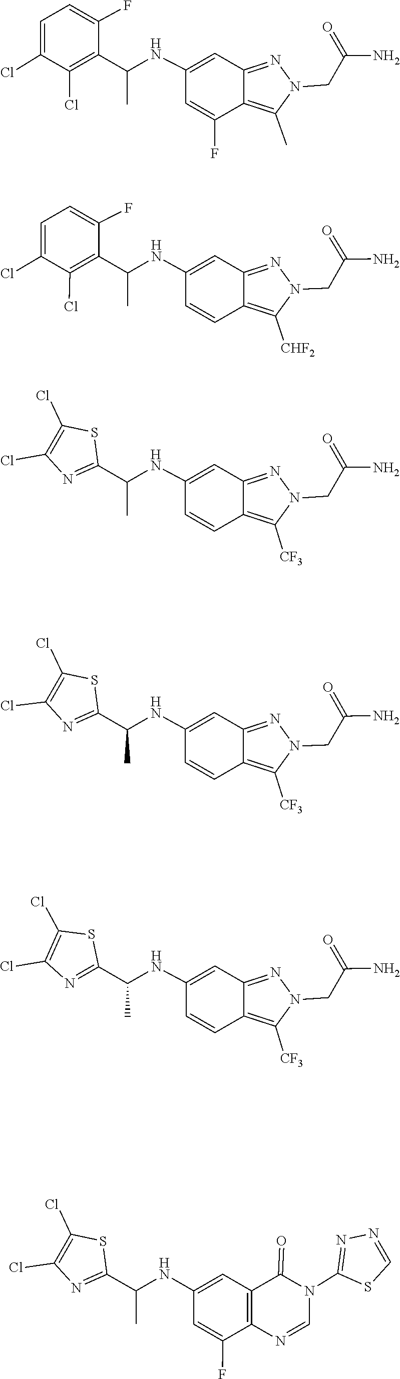 [SMILES: Cc1c2c(F)cc(NC(C)c3c(F)ccc(Cl)c3Cl)cc2nn1CC(N)=O ; CC(Nc1ccc2c(C(F)F)n(CC(N)=O)nc2c1)c1c(F)ccc(Cl)c1Cl ; CC(Nc1ccc2c(C(F)(F)F)n(CC(N)=O)nc2c1)c1nc(Cl)c(Cl)s1 ; C[C@H](Nc1ccc2c(C(F)(F)F)n(CC(N)=O)nc2c1)c1nc(Cl)c(Cl)s1 ; C[C@@H](Nc1ccc2c(C(F)(F)F)n(CC(N)=O)nc2c1)c1nc(Cl)c(Cl)s1 ; CC(Nc1cc(F)c2ncn(-c3nncs3)c(=O)c2c1)c1nc(Cl)c(Cl)s1] 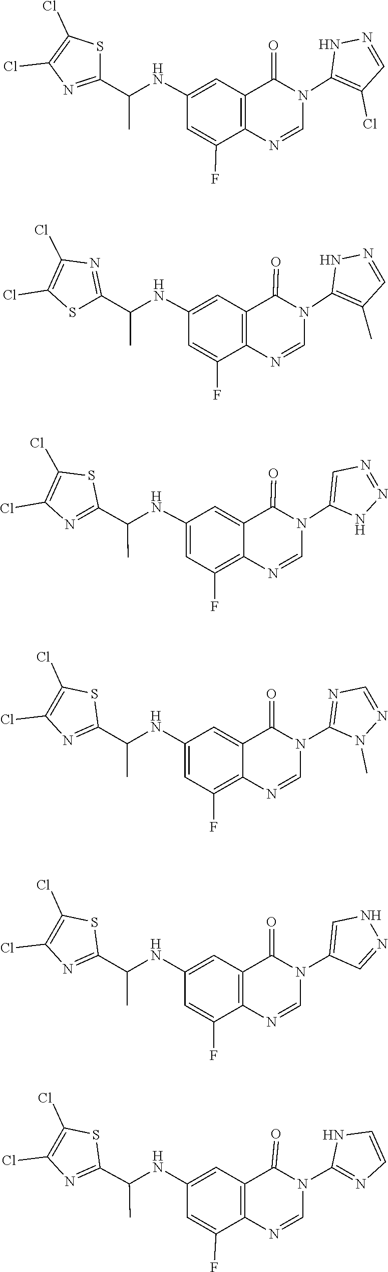 [SMILES: CC(Nc1cc(F)c2ncn(-c3[nH]ncc3Cl)c(=O)c2c1)c1nc(Cl)c(Cl)s1 ; Cc1cn[nH]c1-n1cnc2c(F)cc(NC(C)c3nc(Cl)c(Cl)s3)cc2c1=O ; CC(Nc1cc(F)c2ncn(-c3cnn[nH]3)c(=O)c2c1)c1nc(Cl)c(Cl)s1 ; CC(Nc1cc(F)c2ncn(-c3ncnn3C)c(=O)c2c1)c1nc(Cl)c(Cl)s1 ; CC(Nc1cc(F)c2ncn(-c3cn[nH]c3)c(=O)c2c1)c1nc(Cl)c(Cl)s1 ; CC(Nc1cc(F)c2ncn(-c3ncc[nH]3)c(=O)c2c1)c1nc(Cl)c(Cl)s1] 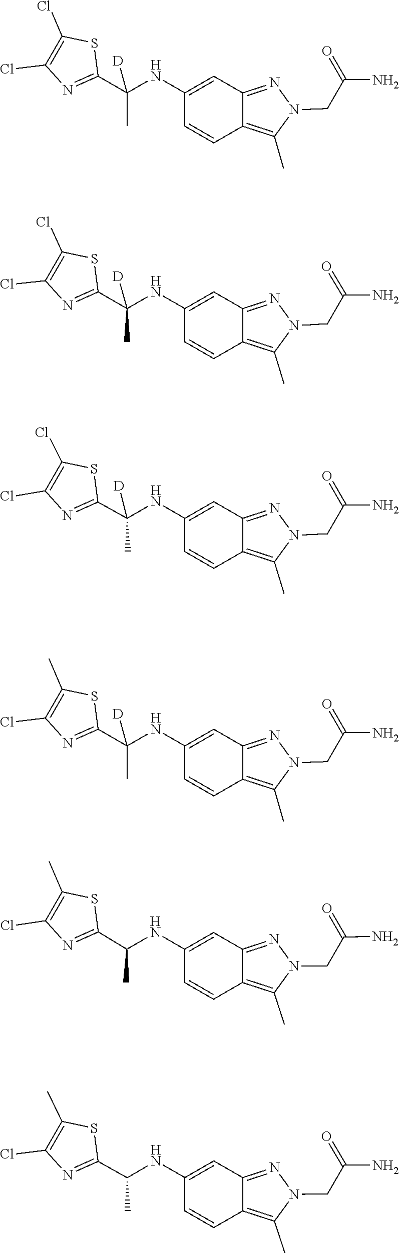 [SMILES: [2H]C(C)(Nc1ccc2c(C)n(CC(N)=O)nc2c1)c1nc(Cl)c(Cl)s1 ; [2H][C@](C)(Nc1ccc2c(C)n(CC(N)=O)nc2c1)c1nc(Cl)c(Cl)s1 ; [2H][C@@](C)(Nc1ccc2c(C)n(CC(N)=O)nc2c1)c1nc(Cl)c(Cl)s1 ; [2H]C(C)(Nc1ccc2c(C)n(CC(N)=O)nc2c1)c1nc(Cl)c(C)s1 ; Cc1sc([C@H](C)Nc2ccc3c(C)n(CC(N)=O)nc3c2)nc1Cl ; Cc1sc([C@@H](C)Nc2ccc3c(C)n(CC(N)=O)nc3c2)nc1Cl] 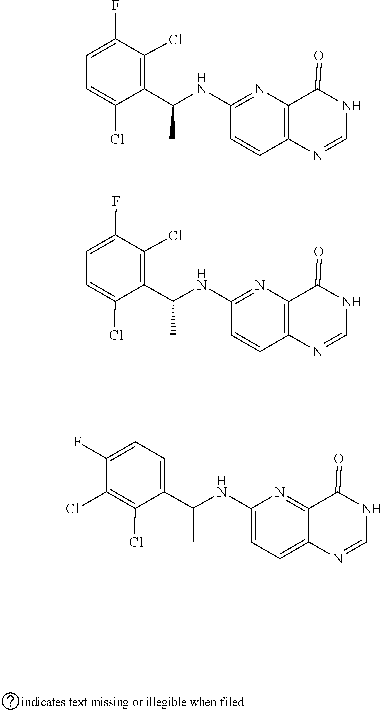 [SMILES: C[C@H](Nc1ccc2nc[nH]c(=O)c2n1)c1c(Cl)ccc(F)c1Cl ; C[C@@H](Nc1ccc2nc[nH]c(=O)c2n1)c1c(Cl)ccc(F)c1Cl ; CC(Nc1ccc2nc[nH]c(=O)c2n1)c1ccc(F)c(Cl)c1Cl] 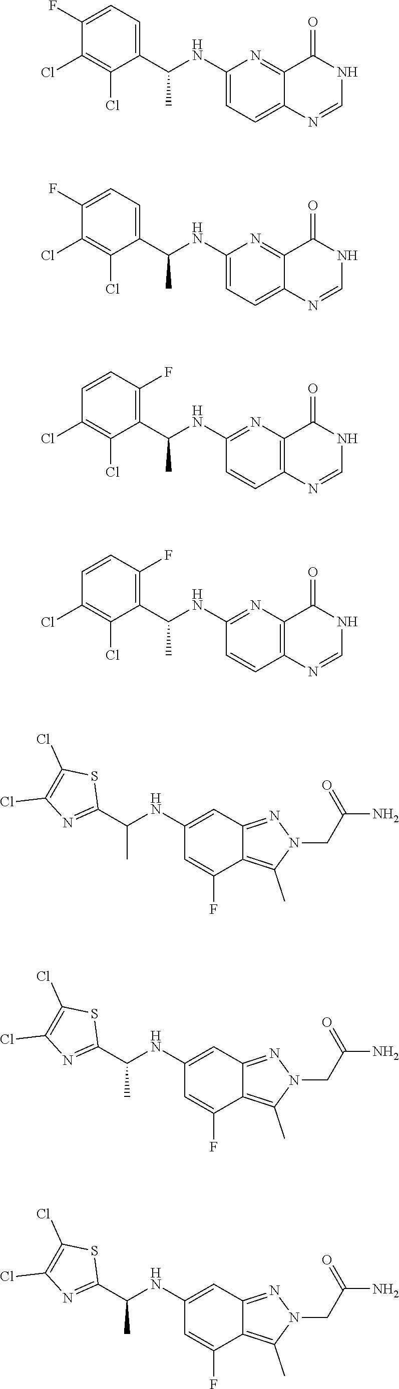
a tautomer thereof, a solvate or stereoisomer of the compound or the tautomer, or a pharmaceutically acceptable salt of the foregoing.

70. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

71. A method of treating a disease or condition, comprising administering to a subject in need thereof, a therapeutically effective amount of a compound according to claim 1, or a compound selected from Compounds 1A to 121A, a tautomer thereof, a solvate or stereoisomer of the compound or the tautomer, or a pharmaceutically acceptable salt of the foregoing, or a pharmaceutical composition comprising the compound according to claim 1, or a pharmaceutical composition comprising any one of Compounds 1A to 121A, wherein the disease or condition is selected from ALS, Parkinson's disease, multiple sclerosis, traumatic brain injury, diabetic neuropathy, and CIPN.

72. A method of treating a disease or condition caused by axonal degeneration, comprising administering to a subject in need thereof, a therapeutically effective amount of a compound according to claim 1 or a compound selected from Compounds 1A to 121A, a tautomer thereof, a solvate or stereoisomer of the compound or the tautomer, or a pharmaceutically acceptable salt of the foregoing, or a pharmaceutical composition comprising the compound according to claim 1, or a pharmaceutical composition comprising any one of Compounds 1A to 121A.

73. A method of modulating SARM1, comprising contacting a subject in need thereof with a compound according to claim 1 or a compound selected from Compounds 1A to 121A, a tautomer thereof, a solvate or stereoisomer of the compound or the tautomer, or a pharmaceutically acceptable salt of the foregoing, or a pharmaceutical composition comprising the compound according to claim 1, or a pharmaceutical composition comprising any one of Compounds 1A to 121A.

74. A method of inhibiting or preventing axonal degeneration, comprising contacting a subject in need thereof with a compound according to claim 1, or a compound selected from Compounds 1A to 121A, a tautomer thereof, a solvate or stereoisomer of the compound or the tautomer, or a pharmaceutically acceptable salt of the foregoing, or a pharmaceutical composition comprising the compound according to claim 1, or a pharmaceutical composition comprising any one of Compounds 1A to 121A.
